(19) United States
(12) Patent Application Publication
(10) Pub. No.: US 2026/0263606 A1
(43) Pub. Date: Sep. 10, 2026

(54) N6-ADENOSINE-METHYLTRANSFERASE PROTACS AND METHODS OF USE THEREOF

(71) Applicant: University of Zurich, Zürich (CH)

(72) Inventors: Francesco ERRANI, Zürich (CH); Annalisa INVERNIZZI, Zürich (CH); Frantisek ZALESAK, Zürich (CH); Marcin HEROK, Zürich (CH); Aymeric DOLBOIS, Shwoben (FR); Danzhi HUANG, Zürich (CH); Amedeo CAFLISCH, Zürich (CH)

(21) Appl. No.: 18/864,671

(22) PCT Filed: May 17, 2023
(86) PCT No.: PCT/EP23/63254

§ 371 Date: Nov. 11, 2024

(30) Foreign Application Priority Data
May 17, 2022 (WO) PCT/EP22/63350
Nov. 16, 2022 (EP) 22207794.3

(51) Int. Cl. A61K 47/55; A61K 47/54; A61P 35/00

(52) U.S. Cl. CPC A61K 47/55; A61K 47/545; A61P 35/00

(57) ABSTRACT

The present disclosure relates to proteolysis targeting chimeras (PROTACs) that modulate N6-adenosine-methyltransferase and methods of using the same.

### CROSS-REFERENCE TO RELATED APPLICATIONS

This application claims the benefit of EP Patent Application No. 22207794.3, filed on Nov. 16, 2022, and International Application No. PCT/EP2022/063350, filed on May 17, 2022, which are incorporated by reference herein in their entireties.

The present disclosure relates to proteolysis targeting chimeras (PROTACs) that modulate N6-adenosine-methyltransferase and methods of using the same.

### BACKGROUND

Expression of genes is regulated at the level of the transcriptome (messenger RNA obtained through transcription of the genome) through dynamic levels of mRNA modifications. The conversion of adenosine to N6-methyladenosine (m⁶A) is the most common internal post-transcriptional modification (also called epitranscriptomic modification) in eukaryotic mRNA.

This methylation event typically occurs within the DRACH (D=A, G, U; R=A, G; H=A, C, U) consensus sequence motif. The m⁶A level can vary among different tissues, development states or in response to cellular stresses. On the molecular level introduction of the m⁶A affects the structure of RNA and its ability to form protein-RNA interactions, and as a consequence it modulates processing, translation, and stability of the cellular transcripts. As a consequence, m⁶A is implicated in controlling embryonic development processes and stem cell differentiation, regulating the mammalian circadian clock, and modulating stress response, e.g., heat shock.

The dynamic level of m⁶A is regulated by the interplay of erasers and writer proteins. While the m⁶A writer has been known for two decades, the discovery of m⁶A-specific eraser proteins FTO (ALKBH9) and ALKBH5 has ultimately demonstrated the reversibility of the modification and its regulatory role. These m⁶A demethylases belong to the dioxygenase AlkB family whose enzymatic reaction depends on Fe(II) and 2-oxoglutaric acid (20G). The core writer complex is formed by two methyltransferase-like proteins, METTL3 and METTL14, which rely on additional cofactors for mRNA substrate recruitment, including WTAP and RBM15. The METTL3-METTL14 complex transfers a methyl group from S-adenosylmethionine (SAM) to the adenosine within the consensus sequence of 5′-GGACU-3′. Only METTL3 has an intact SAM-binding site, while METTL14 possesses a degenerate SAM-binding site, which is not functional. The individual depletion of METTL3 or METTL14 reduces the level of m⁶A in HeLa cells. More importantly, deregulation of METTL3 has recently been linked to specific tumors, such as acute myeloid leukemia, hepatocellular carcinoma, and lung adenocarcinoma. Moreover, inhibiting m⁶A modification shows a broad antiviral effect. Therefore, small-molecule modulators of the METTL3-METTL14 writer has potential therapeutic use in cancer and viral infection.

Based on the above-mentioned state of the art, the objective of the present invention is to provide means and methods to use the PROTAC therapeutic modalities to modulate the levels of m6A modification with the goal of regulating gene expression for cancer therapy. This objective is attained by the subject-matter of the independent claims of the present specification.

### SUMMARY

In one aspect, the present disclosure relates to bifunctional compounds, which find utility to degrade and inhibit N6-adenosine-methyltransferase. In some embodiments, the bifunctional compounds target a heterodimeric complex METTL3-METTL14. In some embodiments, the bifunctional compounds bind to the heterodimeric complex METTL3-METTL14. In some embodiments, the bifunctional compounds modulate the heterodimeric complex METTL3-METTL14. In some embodiments, the bifunctional compounds inhibit and degrade the heterodimeric complex METTL3-METTL14. In some embodiments, the bifunctional compounds target METTL3. In some embodiments, the bifunctional compounds bind to METTL3. In some embodiments, the bifunctional compounds modulate, inhibit, and/or degrade METTL3. In some embodiments, the bifunctional compounds target METTL14. In some embodiments, the bifunctional compounds bind to METTL14. In some embodiments, the bifunctional compounds modulate, inhibit, and/or degrade METTL14.

A first aspect of the disclosure relates to a compound of the general formula (A)

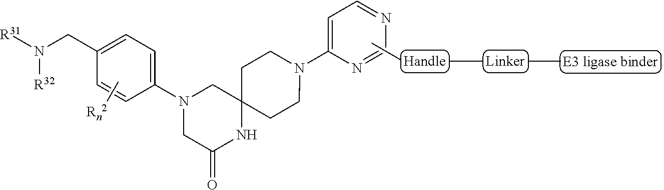

A second aspect of the disclosure relates to a compound of the general formula (U)

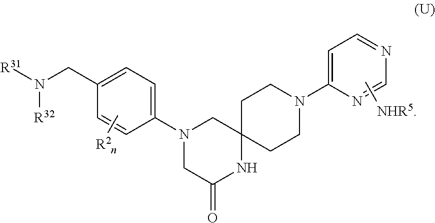

A third aspect of the disclosure relates to a compound according to the first or second aspect for use as a medicament.

A fourth aspect of the disclosure relates to a compound according to the first or second aspect for use in treatment of cancer.

In another embodiment, the present disclosure relates a pharmaceutical composition comprising at least one of the compounds of the present disclosure or a pharmaceutically acceptable salt thereof and at least one pharmaceutically acceptable carrier, diluent or excipient.

### BRIEF DESCRIPTION OF THE DRAWINGS

FIG. 1 shows A) Design of METTL3 inhibitor 2 from hit compound 1. The bonds formed are depicted in red. The IC₅₀ values refer to the biochemical assay based on time resolved-Förster resonance energy transfer (TR-FRET). B) Superimposition of compounds 1 (grey, from X-ray with METTL3, PDB code: 7NHI) and 2 (cyan, drawn in Pymol).

FIG. 2 shows design of compound 8 starting from 5, the newly formed bonds are depicted in red. A) Inhibitor 5 (cyan) in the METTL3 binding site with relevant residues (carbon atoms in grey). The main intermolecular interactions are displayed (yellow dashed lines, PDB code: 7O08). B) Superimposition of inhibitors 5 (cyan) and 7 (green, PDB code: 7O09). C). Superimposition of inhibitors 5 (cyan) and 8 (yellow, PDB code: 7O0L), and interactions of the lactam with the side chain amide of Gln550.

FIG. 3 shows A) Unusual interaction of the fluorine atom of compound 20 with Pro397 amide π-system, PDB code: 7O29. B) Van der Waals contacts between the fluorine atom of inhibitor 21 and the side chains of Ser511 and Tyr406, PDB code: 7O2E.

FIG. 4 shows TR-FRET dose response curves (n≥3) measured for compounds 1, 22 and SAH and chemical structure of the lead compound 22. B) InCELL Pulse dose response curve (n=3) in HEK293T cells at 46° C. D) Dose response curves of m6A/A reduction in polyadenylated RNA fraction in MOLM-13 (n=5) and PC-3 (n=3) cell lines measured by UPLC-MS/MS.

FIG. 5 shows thermal shift assay results. Shown are the first derivative of the melting curves of METTL3/METTL14 for inhibitor 22 or SAH.

FIG. 6 shows thermal shift assay results. Shown are the first derivative of the melting curves of METTL1 for inhibitor 22 or SAH. Compound 22 does not shift the melting temperature of METTL1.

FIG. 7 Exemplary compounds.

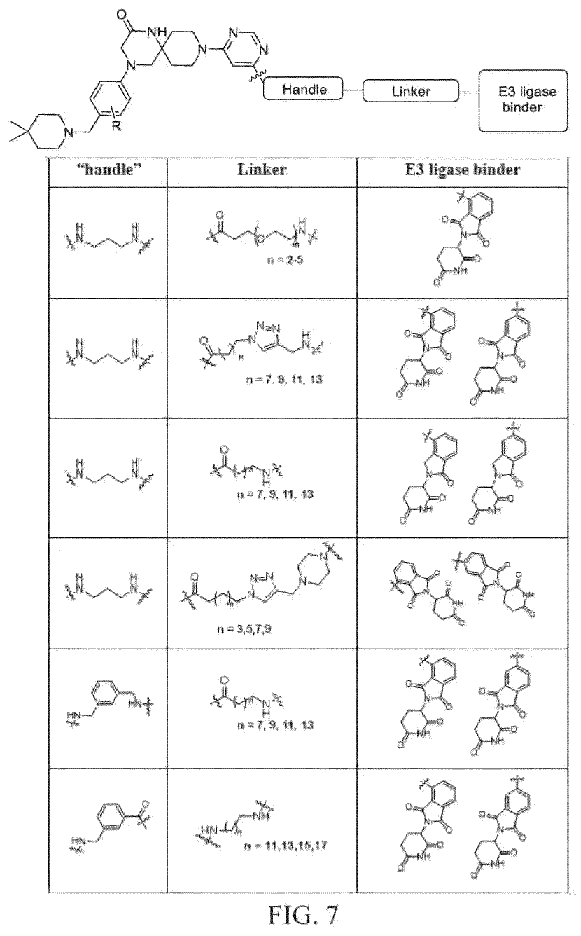

FIG. 8 Exemplary substitution patterns for R.

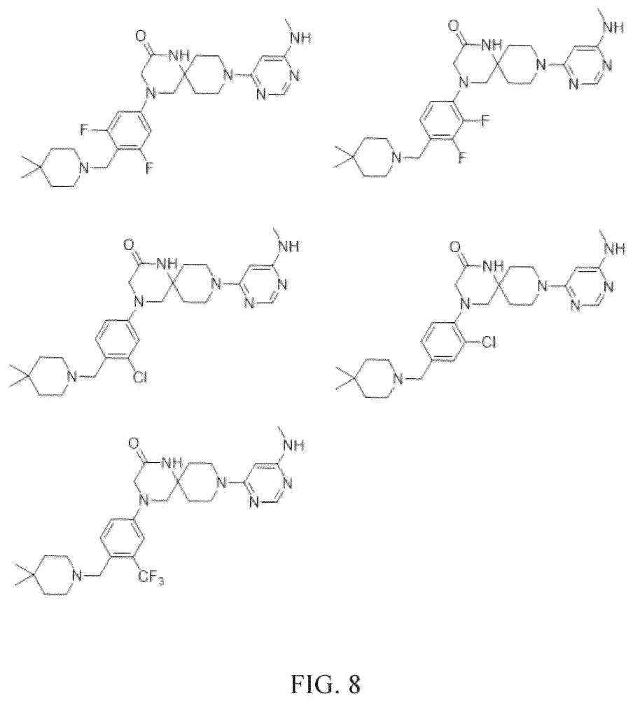

FIG. 9 shows the degradation of METTL3 protein after treatment with 2 μM concentration of various PROTAC molecules for 24h, measured with Western blot.

FIG. 10 shows the METTL14 protein after treatment with 2 μM concentration of various PROTAC molecules for 24h, measured with Western blot.

FIG. 11 shows the correlation between METTL3 and METTL14 degradation of various PROTAC molecules measured by Western blot.

### DETAILED DESCRIPTION

### Terms and Definitions

For purposes of interpreting this specification, the following definitions will apply and whenever appropriate, terms used in the singular will also include the plural and vice versa. In the event that any definition set forth below conflicts with any document incorporated herein by reference, the definition set forth shall control.

The terms “comprising,” “having,” “containing,” and “including,” and other similar forms, and grammatical equivalents thereof, as used herein, are intended to be equivalent in meaning and to be open ended in that an item or items following any one of these words is not meant to be an exhaustive listing of such item or items, or meant to be limited to only the listed item or items. For example, an article “comprising” components A, B, and C can consist of (i.e., contain only) components A, B, and C, or can contain not only components A, B, and C but also one or more other components. As such, it is intended and understood that “comprises” and similar forms thereof, and grammatical equivalents thereof, include disclosure of embodiments of “consisting essentially of” or “consisting of.”

Where a range of values is provided, it is understood that each intervening value, to the tenth of the unit of the lower limit, unless the context clearly dictate otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

Reference to “about” a value or parameter herein includes (and describes) variations that are directed to that value or parameter per se. For example, description referring to “about X” includes description of “X.”

As used herein, including in the appended claims, the singular forms “a,” “or,” and “the” include plural referents unless the context clearly dictates otherwise.

Unless defined otherwise, all technical and scientific terms used herein have the same meaning as commonly understood by one of ordinary skill in the art (e.g., in cell culture, molecular genetics, nucleic acid chemistry, hybridization techniques and biochemistry). Standard techniques are used for molecular, genetic and biochemical methods (see generally, Sambrook et al., Molecular Cloning: A Laboratory Manual, 2d ed. (1989) Cold Spring Harbor Laboratory Press, Cold Spring Harbor, N.Y. and Ausubel et al., Short Protocols in Molecular Biology (1999) 4th Ed, John Wiley & Sons, Inc.) and chemical methods.

The term METTL3 in the context of the present specification relates to N6-adenosine-methyltransferase catalytic subunit (Uniprot ID: Q86U44).

The term METTL14 in the context of the present specification relates to N6-adenosine-methyltransferase non-catalytic subunit (Uniprot ID: Q9HCE5).

A C₁-C₆ alkyl in the context of the present specification signifies a saturated linear or branched hydrocarbon having 1, 2, 3, 4, 5 or 6 carbon atoms. In certain embodiments, the alkyl is substituted, meaning e.g. one or more CH₂ moieties may be exchanged for oxygen (ether bridge) or nitrogen (NH, or NR with R being methyl, ethyl, or propyl; amino bridge).

The term C₃-C₇ cycloalkyl in the context of the present specification relates to a saturated hydrocarbon ring having 3, 4, 5, 6 or 7 carbon atoms, wherein in certain embodiments, one carbon-carbon bond may be unsaturated. Non-limiting examples of a C₃-C₇ cycloalkyl moiety include cyclopropanyl (—C₃H₅), cyclobutanyl (—C₄H₇), cyclopentenyl (C₅H₉), and cyclohexenyl (C₆Hu) moieties. In certain embodiments, the cycloalkyl is substituted. In certain embodiments, a cycloalkyl is substituted by one C₁ to C₄ unsubstituted alkyl moiety.

In certain embodiments, a cycloalkyl is substituted by more than one C₁ to C₄ unsubstituted alkyl moieties.

The term heterocycle in the context of the present specification relates to a cycloalkyl, wherein at least one ring atom is replaced or several ring atoms are replaced by a nitrogen, oxygen and/or sulphur atom.

The term heterobicycle in the context of the present specification relates to two directly connected cycloalkyls, wherein at least one ring atom is replaced or several ring atoms are replaced by a nitrogen, oxygen and/or sulphur atom.

The term heterocycloalkyl in the context of the present specification relates to a cycloalkyl, wherein at least one ring atom is replaced or several ring atoms are replaced by a nitrogen, oxygen and/or sulphur atom.

The term unsubstituted Cₙ alkyl when used herein in the narrowest sense relates to the moiety —CₙH₂ₙ— if used as a bridge between moieties of the molecule, or —CₙH₂ₙ₊₁ if used in the context of a terminal moiety.

The terms unsubstituted Cₙ alkyl and substituted Cₙ alkyl include a linear alkyl comprising or being linked to a cyclical structure, for example a cyclopropane, cyclobutane, cyclopentane or cyclohexane moiety, unsubstituted or substituted depending on the annotation or the context of mention, having linear alkyl substitutions. The total number of carbon and—where appropriate—N, O or other hetero atom in the linear chain or cyclical structure adds up to n.

Where used in the context of chemical formulae, the following abbreviations may be used: Me is methyl CH₃, Et is ethyl —CH₂CH₃, Prop is propyl —(CH₂)₂CH₃ (n-propyl, n-pr) or —CH(CH₃)₂ (iso-propyl, i-pr), but is butyl —C₄H₉, —(CH₂)₃CH₃, —CHCH₃CH₂CH₃, —CH₂CH(CH₃)₂ or —C(CH₃)₃.

The term substituted alkyl in its broadest sense refers to an alkyl as defined above in the broadest sense, which is covalently linked to an atom that is not carbon or hydrogen, particularly to an atom selected from N, O, F, B, Si, P, S, Cl, Br and I, which itself may be—if applicable—linked to one or several other atoms of this group, or to hydrogen, or to an unsaturated or saturated hydrocarbon (alkyl or aryl in their broadest sense). In a narrower sense, substituted alkyl refers to an alkyl as defined above in the broadest sense that is substituted in one or several carbon atoms by groups selected from amine NH₂, alkylamine NHR, imide NH, alkylimide NR, amino(carboxyalkyl) NHCOR or NRCOR, hydroxyl OH, oxyalkyl OR, oxy(carboxyalkyl) OCOR, carbonyl O and its ketal or acetal (OR)₂, nitril CN, isonitril NC, cyanate CNO, isocyanate NCO, thiocyanate CNS, isothiocyanate NCS, fluoride F, choride Cl, bromide Br, iodide I, phosphonate PO₃H₂, PO₃R₂, phosphate OPO₃H₂ and OPO₃R₂, sulfhydryl SH, suflalkyl SR, sulfoxide SOR, sulfonyl SO₂R, sulfanylamide SO₂NHR, sulfate SO₃H and sulfate ester SO₃R with R being defined further in the description.

The term hydroxyl substituted group refers to a group that is modified by one or several hydroxyl groups OH.

The term amino substituted group refers to a group that is modified by one or several amino groups NH₂.

The term carboxyl substituted group refers to a group that is modified by one or several carboxyl groups COOH.

Non-limiting examples of amino-substituted alkyl include —CH₂NH₂, —CH₂NHMe, —CH₂NHEt, —CH₂CH₂NH₂, —CH₂CH₂NHMe, —CH₂CH₂NHEt, —(CH₂)₃NH₂, —(CH₂)₃NHMe, —(CH₂)₃NHEt, —CH₂CH(NH₂)CH₃, —CH₂CH(NHMe)CH₃, —CH₂CH(NHEt)CH₃, —(CH₂)₃CH₂NH₂, —(CH₂)₃CH₂NHMe, —(CH₂)₃CH₂NHEt, —CH(CH₂NH₂)CH₂CH₃, —CH(CH₂NHMe)CH₂CH₃, —CH(CH₂NHEt)CH₂CH₃, —CH₂CH(CH₂NH₂)CH₃, —CH₂CH(CH₂NHMe)CH₃, —CH₂CH(CH₂NHEt)CH₃, —CH(NH₂)(CH₂)₂NH₂, —CH(NHMe)(CH₂)₂NHMe, —CH(NHEt)(CH₂)₂NHEt, —CH₂CH(NH₂)CH₂NH₂, —CH₂CH(NHMe)CH₂NHMe, —CH₂CH(NHEt)CH₂NHEt, —CH₂CH(NH₂)(CH₂)₂NH₂, —CH₂CH(NHMe)(CH₂)₂NHMe, —CH₂CH(NHEt)(CH₂)₂NHEt, —CH₂CH(CH₂NH₂)₂, —CH₂CH(CH₂NHMe)₂ and —CH₂CH(CH₂NHEt)₂ for terminal moieties and —CH₂CHNH₂—, —CH₂CHNHMe-, —CH₂CHNHEt- for an amino substituted alkyl moiety bridging two other moieties.

Non-limiting examples of hydroxy-substituted alkyl include —CH₂OH, —(CH₂)₂OH, —(CH₂)₃OH, —CH₂CH(OH)CH₃, —(CH₂)₄OH, —CH(CH₂OH)CH₂CH₃, —CH₂CH(CH₂OH)CH₃, —CH(OH)(CH₂)₂OH, —CH₂CH(OH)CH₂OH, —CH₂CH(OH)(CH₂)₂OH and —CH₂CH(CH₂OH)₂ for terminal moieties and —CHOH—, —CH₂CHOH—, —CH₂CH(OH)CH₂—, —(CH₂)₂CHOHCH₂—, —CH(CH₂OH)CH₂CH₂—, —CH₂CH(CH₂OH)CH₂—, —CH(OH)(CH₂CHOH—, —CH₂CH(OH)CH₂OH, —CH₂CH(OH)(CH₂)₂OH and —CH₂CHCH₂OHCHOH— for a hydroxyl substituted alkyl moiety bridging two other moieties.

Haloalkyl refers to an alkyl radical, as defined above, that is substituted by one or more halo radicals, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like.

The term sulfoxyl substituted group refers to a group that is modified by one or several sulfoxyl groups —SO₂R, or derivatives thereof, with R being defined further in the description.

The term sulfonamide substituted group refers to a group that is modified by one or several sulfonamide groups —SO₂NHR or —NHSO₂R, or derivatives thereof, with R being defined further in the description.

The term amine substituted group refers to a group that is modified by one or several amine groups —NHR or —NR₂, or derivatives thereof, with R being defined further in the description.

The term carbonyl substituted group refers to a group that is modified by one or several carbonyl groups —COR, or derivatives thereof, with R being defined further in the description.

An ester refers to a group of —CO₂R, with R being defined further in the description.

An ether refers to a group having one oxygen in between two saturated carbon atoms.

An amide refers to a group of —CONHR, with R being defined further in the description.

An ethylene glycol refers to a group of —(CH₂—CH₂—O)ₙ— or —(O—CH₂—CH₂)ₙ—, with n being defined further in the description.

An alkylyne refers to a group of —CΞC— (triple bond between two carbon atoms).

An alkylene refers to a group of —CH═CH— (double bond between two carbon atoms).

The term halogen-substituted group refers to a group that is modified by one or several halogen atoms selected (independently) from F, Cl, Br, I.

The term fluoro substituted alkyl refers to an alkyl according to the above definition that is modified by one or several fluoride groups F. Non-limiting examples of fluoro-substituted alkyl include —CH₂F, —CHF₂, —CF₃, —(CH₂)₂F, —(CHF)₂H, —(CHF)₂F, —C₂F₅, —(CH₂)₃F, —(CHF)₃H, —(CHF)₃F, —C₃F₇, —(CH₂)₄F, —(CHF)₄H, —(CHF)₄F and —C₄F₉.

Non-limiting examples of hydroxyl- and fluoro-substituted alkyl include —CHFCH₂OH, —CF₂CH₂OH, —(CHF)₂CH₂OH, —(CF₂)₂CH₂OH, —(CHF)₃CH₂OH, —(CF₂)₃CH₂OH, —(CH₂)₃OH, —CF₂CH(OH)CH₃, —CF₂CH(OH)CF₃, —CF(CH₂OH)CHFCH₃, and —CF(CH₂OH)CHFCF₃.

The term aryl in the context of the present specification signifies a cyclic aromatic C₅-C₁₀ hydrocarbon. Examples of aryl include, without being restricted to, phenyl and naphthyl.

An alkylaryl in the context of the present specification relates to an alkyl group substituted by an aryl moiety. Particular examples are ethylphenyl, propylphenyl, butylphenyl and their higher homologues. A substituted alkyl aryl may be substituted by the substituent indicated on the alkyl part, if chemically feasible, or on the aryl part of the moiety.

A heteroaryl is an aryl that comprises one or several nitrogen, oxygen and/or sulphur atoms. Examples for heteroaryl include, without being restricted to, pyrrole, thiophene, furan, imidazole, pyrazole, thiazole, oxazole, pyridine, pyrimidine, thiazin, quinoline, benzofuran and indole. A heteroaryl also encompasses a bicyclic heteroaryl. An aryl or a heteroaryl in the context of the specification additionally may be substituted by one or more alkyl groups.

An alkylheteroaryl in the context of the present specification relates to an alkyl group substituted by a heteroaryl moiety.

As used herein, the term pharmaceutical composition refers to a compound of the disclosure, or a pharmaceutically acceptable salt thereof, together with at least one pharmaceutically acceptable carrier. In certain embodiments, the pharmaceutical composition according to the disclosure is provided in a form suitable for topical, parenteral or injectable administration.

As used herein, the term pharmaceutically acceptable carrier includes any solvents, dispersion media, coatings, surfactants, antioxidants, preservatives (for example, antibacterial agents, antifungal agents), isotonic agents, absorption delaying agents, salts, preservatives, drugs, drug stabilizers, binders, excipients, disintegration agents, lubricants, sweetening agents, flavoring agents, dyes, and the like and combinations thereof, as would be known to those skilled in the art (see, for example, Remington: the Science and Practice of Pharmacy, ISBN 0857110624).

As used herein, the term treating or treatment of any disease or disorder (e.g. cancer) refers in one embodiment, to ameliorating the disease or disorder (e.g. slowing or arresting or reducing the development of the disease or at least one of the clinical symptoms thereof). In another embodiment “treating” or “treatment” refers to alleviating or ameliorating at least one physical parameter including those which may not be discernible by the patient. In yet another embodiment, “treating” or “treatment” refers to modulating the disease or disorder, either physically, (e.g., stabilization of a discernible symptom), physiologically, (e.g., stabilization of a physical parameter), or both. Methods for assessing treatment and/or prevention of disease

### PROTAC Compound

In one aspect, disclosed herein are PROTAC compounds that inhibit and/or degrade the heterodimeric complex METTL3-METTL14. In one aspect, disclosed herein are PROTAC compounds that comprise (i) an active compound that target the METTL3-METTL14 complex (see e.g., paragraphs starting with the subheading “Active Compound of the PROTAC”), and (ii) an E3 ligase binder (see e.g., paragraphs starting with the subheading “E3 ligase binder”). In some embodiments, the PROTAC compounds further comprise a Handle (see e.g., paragraphs starting with the subheading “Handle”). In some embodiments, the PROTAC compounds further comprise a Linker (see e.g., paragraphs starting with the subheading “Linker”).

A first aspect of the disclosure relates to a compound of the general formula (A)

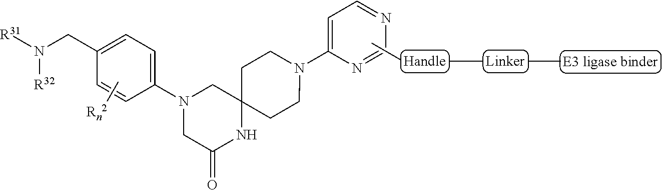

wherein
   NR³¹R³² is selected from an optionally substituted 3-12 membered heterocycloalkyl

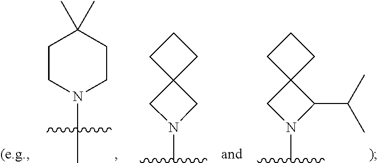

each R² is independently selected from the group consisting of halogen (e.g., F, Cl), C₁-C₃ alkyl, and C₁-C₃ haloalkyl (e.g., CF₃, CHF₂, CH₂F);
n is an integer selected from 0, 1, 2, 3, and 4;
Handle is a connecting moiety comprising or essentially consisting of 3 to 10 atoms of atomic mass ≥12 (C, N, O, S);
Linker is a linker moiety comprising or essentially consisting of 3 to 50 atoms of atomic mass ≥12;
E3 ligase binder is a moiety that binds to an E3 ligase.

A first aspect of the disclosure relates to a compound of the general formula (A)

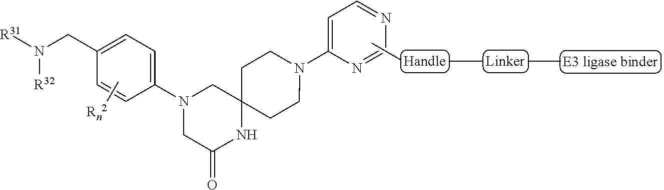

wherein
   NR³¹R³² is selected from

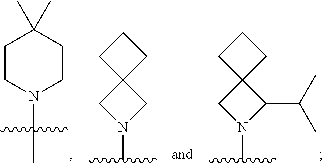

each R² is independently selected from the group comprising F, Cl, CF₃, CHF₂, CH₂F;
n is an integer selected from 0, 1, 2, 3, and 4;
Handle is a connecting moiety comprising or essentially consisting of 3 to 10 atoms of atomic mass ≥12 (C, N, O, S);
Linker is a linker moiety comprising or essentially consisting of 3 to 50 atoms of atomic mass ≥12;
E3 ligase binder is a moiety specifically binding to an E3 ligase.

In another aspect, the disclosure relates to a compound of the general formula (A-1)

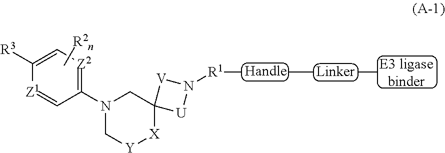

wherein
   Z¹ and Z² are independently selected from N, CH and CR²;
   X is O or NH;
   Y is CH₂, C═O, or SO₂;
   R¹ is an unsubstituted or substituted moiety selected from aryl, heteroaryl, cycloalkyl, and a heterocycle, particularly R¹ is unsubstituted or substituted heteroaryl;
   R² is selected from F, Me, Cl, OH, NH₂, Br, CF₃, CHF₂, CH₂F;
   n is an integer selected from 0, 1, 2, 3, and 4;
   R³ is a substituted alkylamine;
   U and V are independently selected from —CH₂— and —(CH₂)₂—, or one of U and V is —CH₂— and the other one is —(CH₂)₃—;
   Handle is a connecting moiety comprising or essentially consisting of 3 to 10 atoms of atomic mass ≥12 (C, N, O, S);
   Linker is a linker moiety comprising or essentially consisting of 3 to 50 atoms of atomic mass ≥12;
   E3 ligase binder is a moiety that binds to an E3 ligase.

In another aspect, the compound of formula (A-1) has a structure of Formula (A-1a),

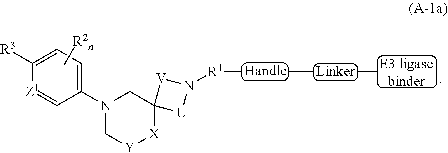

In another aspect, the compound of formula (A-1) has a structure of Formula (A-1b),

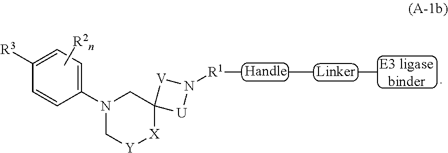

In some embodiments, R¹ has a structure of

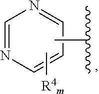

wherein
each R⁴ is independently selected from
   a secondary amine substituted with an alkyl, an alkylaryl, a heteroalkylaryl, a cycloalkyl, an aryl, a heteroaryl and/or a heterocycle,
   a halogen;
and/or two R⁴ together form an unsubstituted or substituted heteroaryl or heterocycle; and
m is an integer selected from 0, 1, 2, and 3.

In some embodiments of Formula (A), (A-1), (A-1a) or (A-1b), R³ is

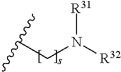

wherein
   s is an integer selected from 1 and 2, more particularly s is 1;
   R³¹ and R³² together form a heterocycle or heterobicycle, which is unsubstituted or alkyl-, halogen-, and/or hydroxyl-substituted, or R³¹ and R³² are independently selected from hydrogen and unsubstituted or hydroxy-, and/or halogen-substituted alkyl or cycloalkyl.

In some embodiments of Formula (A), (A-1), (A-1a) or (A-1b), R³ has a structure of —CH₂—NR³¹R³². In some embodiments, NR³¹R³² is selected from

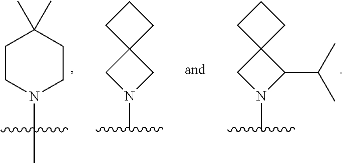

In some embodiments of Formula (A), (A-1), (A-1a) or (A-1b), NR³¹R³² is selected from

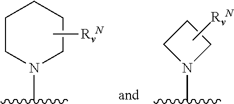

with v being an integer selected from 0, 1 and 2 and each R^{N} being independently selected from hydroxyl, halogen, and C₁-C₄ alkyl, or two R^{N} form a C₃-C₆ cycloalkyl.

In some embodiments,

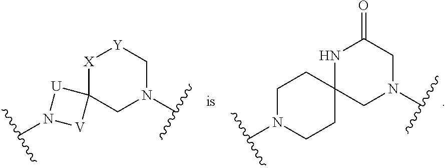

In certain embodiments, each R² is F. In certain embodiments, n is an integer selected from 0, 1, and 2. In certain embodiments, n is 2. In certain embodiments, Handle is a connecting moiety comprising or essentially consisting of 4 to 8 atoms of atomic mass ≥12. In certain embodiments, Linker is a linker moiety comprising or essentially consisting of 4 to 30 atoms of atomic mass ≥12. In certain embodiments, Linker is a linker moiety comprising or essentially consisting of 5 to 20 atoms of atomic mass ≥12.

Further embodiments of the various groups of the disclosed PROTAC compounds (e.g., R¹, R², R³, R³¹, R³², R⁴, Handle, and Linker) are further described in the sections below.

### E3 Ligase Binder

An E3 ligase binder is a molecule which specifically binds an E3 ligase. In certain embodiments, the E3 ligase is cereblon (UniProt-ID: Q96SW2).

In certain embodiments, the E3 ligase binder is of the formula (B)

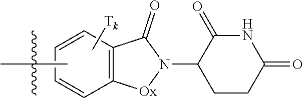

wherein
   Ox is CH₂ or C═O;
   T is selected from the group comprising F, Cl;
   k is an integer selected from the group comprising 0, 1, 2;
   [symbol] designates the bond to the Linker.

In certain embodiments, k is an integer selected from the group comprising 0, 1. In certain embodiments, k is 0. In certain embodiments, T is F.

### Handle

In certain embodiments, Handle is a connecting moiety comprising or essentially consisting of 3 to 10 atoms of atomic mass ≥12 (C, N, O, S). In certain embodiments, Handle is a connecting moiety comprising or essentially consisting of 4 to 8 atoms of atomic mass ≥12. In certain embodiments, Handle is a connecting moiety comprising or essentially consisting of 5 to 10 atoms of atomic mass ≥12. In certain embodiments, Handle is a connecting moiety comprising or essentially consisting of 5 to 15 atoms of atomic mass ≥12. In certain embodiments, Handle is a connecting moiety comprising or essentially consisting of 10 to 15 atoms of atomic mass ≥12. In certain embodiments, Handle is a connecting moiety comprising or essentially consisting of 5 to 20 atoms of atomic mass ≥12.

In certain embodiments, the Handle comprises or essentially consists of 1, 2, 3, or 4 chemical moieties selected from the group comprising alkyl, amine, phenyl, and carbonyl.

In certain embodiments, the Handle is selected from the group comprising the following formulas:

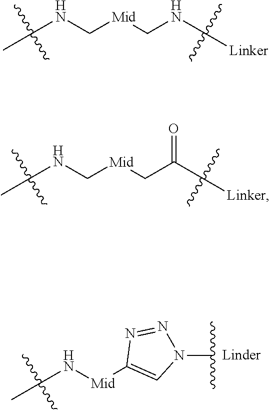

wherein
   Mid is selected from the group comprising C₁-C₃ alkyl, and phenyl.

In certain embodiments, the Handle is Formula (X)

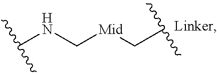

wherein Mid is selected from the group comprising C₁-C₃ alkyl, and phenyl.

In certain embodiments, the Handle is Formula (Y):

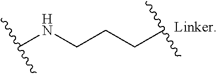 [SMILES: *CCCN*]

In certain embodiments, the Handle is selected from the group comprising the following formulas:

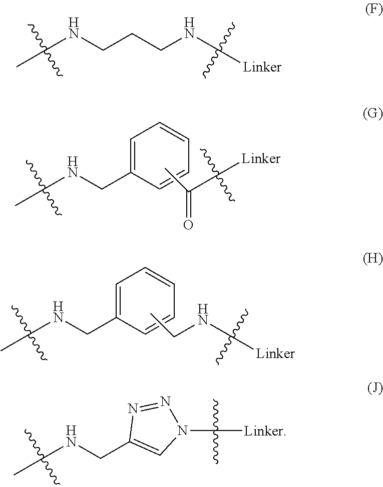

### Linker

In certain embodiments, Linker is a linker moiety comprising or essentially consisting of 3 to 50 atoms of atomic mass ≥12 (C, N, O, S). In certain embodiments, Linker is a linker moiety comprising or essentially consisting of 4 to 30 atoms of atomic mass ≥12. In certain embodiments, Linker is a linker moiety comprising or essentially consisting of 5 to 20 atoms of atomic mass ≥12. In certain embodiments, Linker is a linker moiety comprising or essentially consisting of 5 to 10 atoms of atomic mass ≥12. In certain embodiments, Linker is a linker moiety comprising or essentially consisting of 5 to 15 atoms of atomic mass ≥12. In certain embodiments, Linker is a linker moiety comprising or essentially consisting of 5 to 25 atoms of atomic mass ≥12. In certain embodiments, Linker is a linker moiety comprising or essentially consisting of 10 to 15 atoms of atomic mass ≥12.

In certain embodiments, Linker and Handle, as combined, is a linker moiety comprising or essentially consisting of 5 to 25 atoms of atomic mass ≥12. In certain embodiments, Linker and Handle, as combined, is a linker moiety comprising or essentially consisting of 4 to 35 atoms of atomic mass ≥12. In certain embodiments, Linker and Handle, as combined, is a linker moiety comprising or essentially consisting of 4 to 25 atoms of atomic mass ≥12. In certain embodiments, Linker and Handle, as combined, is a linker moiety comprising or essentially consisting of 15 to 25 atoms of atomic mass ≥12. In certain embodiments, Linker and Handle, as combined, is a linker moiety comprising or essentially consisting of 12 to 30 atoms of atomic mass ≥12. In certain embodiments, Linker and Handle, as combined, is a linker moiety comprising or essentially consisting of 16 to 22 atoms of atomic mass ≥12.

In certain embodiments, the Linker comprises or essentially consists of 1, 2, 3, 4, 5, 6, or 7 chemical moieties independently selected from the group comprising alkyl, aryl, heteroaryl, cycloalkyl, heterocycloalkyl, alkylene, alkylyne, ethylene glycol, carbonyl, ether, ester, amine, amide, sulfonamide, wherein the chemical moieties are each independently unsubstituted or substituted with C₁-C₃ alkyl, halogen, CN, NO₂, hydroxyl, amine, sulfate, phosphate, and/or carboxyl.

In certain embodiments, the Linker comprises or essentially consists of 1, 2, 3, or 4 chemical moieties selected from the group comprising alkyl, ethylene glycol, carbonyl, piperazine, aryl, amine, triazole.

In certain embodiments, the Linker is selected from the group comprising the following formulas:

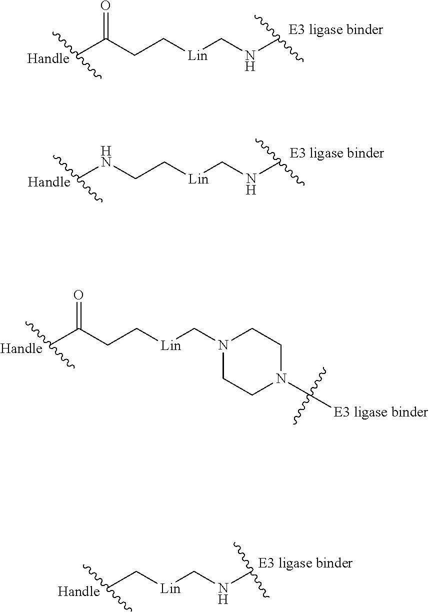

wherein
   Lin is selected from the group comprising C₃-C₂₀ alkyl, C₃-C₂₀ alkyl-triazole, oligo(ethylene glycol).

In certain embodiments, the Linker is selected from the group comprising the following formulas:

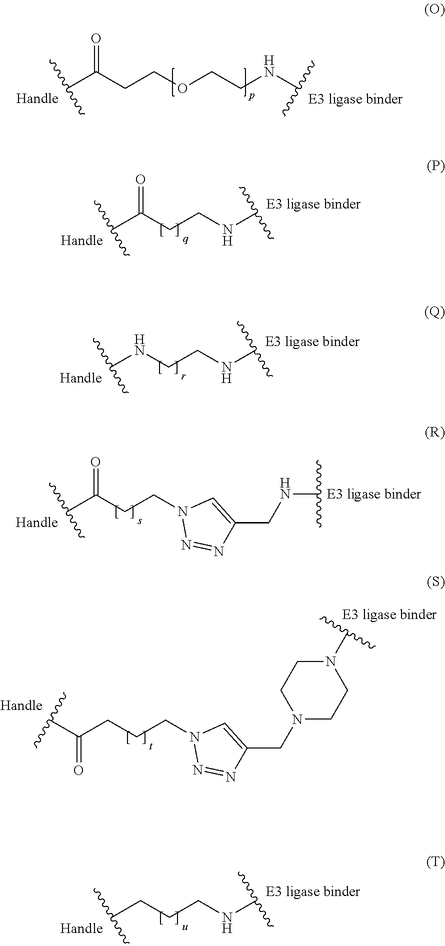

wherein
   p is selected from 2, 3, 4, and 5;
   q is selected from 7, 8, 9, 10, 11, 12, and 13;
   r is selected from 11, 12, 13, 14, 15, 16, and 17;
   s is selected from 7, 8, 9, 10, 11, 12, and 13;
   t is selected from 3, 4, 5, 6, 7, 8, and 9;
   u is selected from 7, 8, 9, 10, 11, 12, 13, 14, 15, 16, 17, 18, 19, and 20.

In certain embodiments, the Linker is selected from the group consisting of the following formulas (O), (P), (Q), (R), (S), and (T), wherein
p is selected from 2, 3, 4, and 5;
q is selected from 7, 8, 9, 10, 11, 12, and 13;
r is selected from 11, 12, 13, 14, 15, 16, and 17;
s is selected from 7, 8, 9, 10, 11, 12, and 13;
t is selected from 3, 4, 5, 6, 7, 8, and 9; and
u is selected from 7, 8, 9, 10, 11, 12, and 13.

In certain embodiments, the Linker is selected from the group consisting of the following formulas (O), (P), (Q), (R), (S), and (T), wherein
p is selected from 2, 3, 4, and 5;
q is selected from 7, 8, 9, 10, 11, 12, and 13;
r is selected from 11, 12, 13, 14, 15, 16, and 17;
s is selected from 7, 8, 9, 10, 11, 12, and 13;
t is selected from 3, 4, 5, 6, 7, 8, and 9; and
u is selected from 7, 8, 9, 10, 11, 12, 13, 14 and 15.

In certain embodiments, the Linker is selected from the group comprising the following formulas:

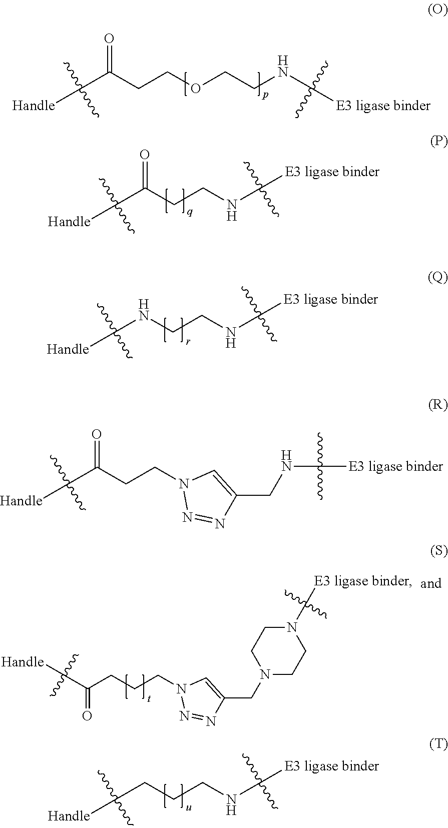

wherein
   p is selected from 2, 3, 4, and 5;
   q is selected from 7, 8, 9, 10, 11, 12, and 13;
   r is selected from 11, 12, 13, 14, 15, 16, and 17;
   s is selected from 7, 8, 9, 10, 11, 12, and 13;
   t is selected from 3, 4, 5, 6, 7, 8, and 9;
   u is selected from 7, 8, 9, 10, 11, 12, 13, 14, 15, 16, 17, 18, 19, and 20.

In certain embodiments, the Linker is selected from the group consisting of the following formulas (O), (P), (Q), (R), (S), (T), and (V) wherein
p is selected from 2, 3, 4, and 5;
q is selected from 7, 8, 9, 10, 11, 12, and 13;
r is selected from 11, 12, 13, 14, 15, 16, and 17;
s is selected from 7, 8, 9, 10, 11, 12, and 13;
t is selected from 3, 4, 5, 6, 7, 8, and 9; and
u is selected from 7, 8, 9, 10, 11, 12, and 13.

In certain embodiments, the Linker is selected from the group consisting of the following formulas (O), (P), (Q), (R), (S), (T), and (V) wherein
p is selected from 2, 3, 4, and 5;
q is selected from 7, 8, 9, 10, 11, 12, and 13;
r is selected from 11, 12, 13, 14, 15, 16, and 17;
s is selected from 7, 8, 9, 10, 11, 12, and 13;
t is selected from 3, 4, 5, 6, 7, 8, and 9; and
u is selected from 7, 8, 9, 10, 11, 12, 13, 14 and 15.

In certain embodiments, the Linker is a peptide. In certain embodiments, the Linker is a peptide consisting of proteinogenic amino acids.

In certain embodiments, the Linker comprises Formula (W):

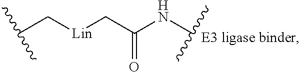

wherein Lin is selected from the group comprising C₃-C₂₀ alkyl, C₃-C₂₀ alkyl-triazole, and oligo(ethylene glycol).

In certain embodiments, the Linker comprises Formula (Z):

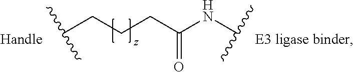

wherein z is selected from 4, 5, 6, 7, 8, 9, and 10.

### Combination of Features

In certain embodiments,
the E3 ligase binder is of the formula (B); and
the Handle is of formula (F), (G), (H), or (J); and
the Linker is of formula (0); (P); (Q); (R); (S); or (T).

In certain embodiments,
the E3 ligase binder is of the formula (B); and
the Handle is of formula (X) or (Y); and
the Linker is of formula (0); (P); (Q); (R); (S), (T), or (V).

In certain embodiments,
the E3 ligase binder is of the formula (B); and
the Handle is of formula (C), (D), (E); and
the Linker is of formula (W) or (Z).

In certain embodiments, the compound comprises the definitions of Handle, Linker, and E3 ligase binder (one row is one combination) according to Table 1:

TABLE 1

| Handle | | Linker |
|---|---|---|
| 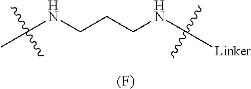 [SMILES: *NCCCN* ; "(F)"] | | 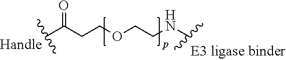 (O) |
| 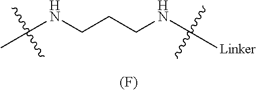 [SMILES: *NCCCN* ; "(F)"] | | 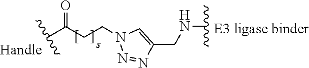 (R) |
| 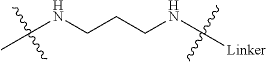 (F) | | 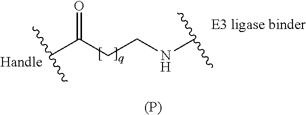 |
| 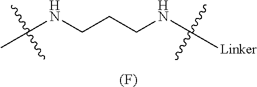 [SMILES: *NCCCN* ; "(F)"] | | 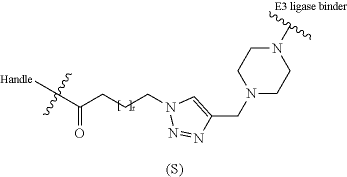 |
| 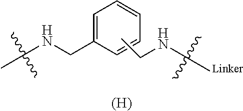 | | 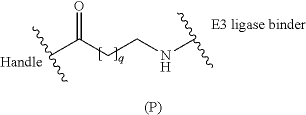 |
| 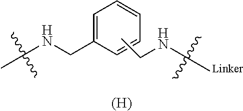 | | 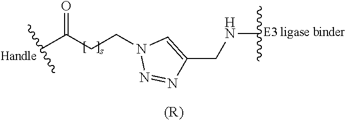 |
| 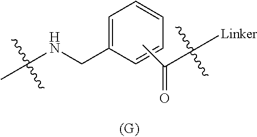 | | 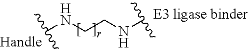 (Q) |
| 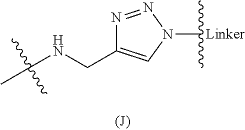 [SMILES: *NCc1cn(*)nn1] | | 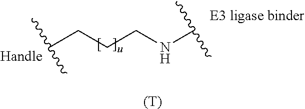 |
| | Handle | E3 ligase binder |
| | 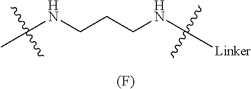 [SMILES: *NCCCN* ; "(F)"] | 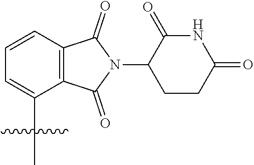 [SMILES: *c1cccc2c1C(=O)N(C1CCC(=O)NC1=O)C2=O] |
| | 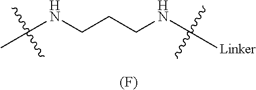 [SMILES: *NCCCN* ; "(F)"] | 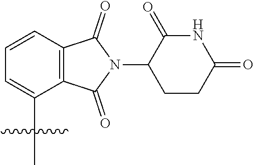 [SMILES: *c1cccc2c1C(=O)N(C1CCC(=O)NC1=O)C2=O] |
| | | 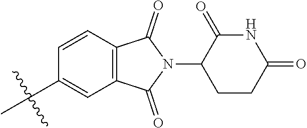 [SMILES: *c1ccc2c(c1)C(=O)N(C1CCC(=O)NC1=O)C2=O] |
| | 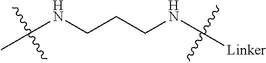 (F) | 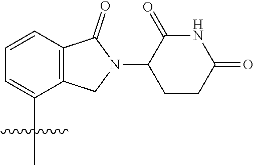 [SMILES: *c1cccc2c1CN(C1CCC(=O)NC1=O)C2=O] |
| | | 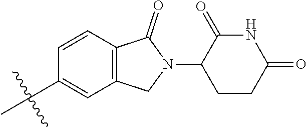 [SMILES: *c1ccc2c(c1)CN(C1CCC(=O)NC1=O)C2=O] |
| | 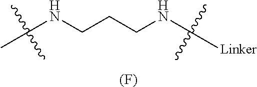 [SMILES: *NCCCN* ; "(F)"] | 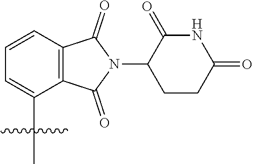 [SMILES: *c1cccc2c1C(=O)N(C1CCC(=O)NC1=O)C2=O] |
| | | 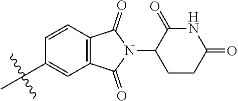 [SMILES: *c1ccc2c(c1)C(=O)N(C1CCC(=O)NC1=O)C2=O] |
| | 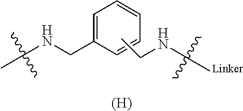 | 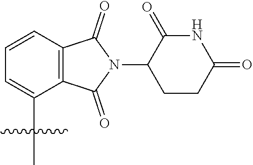 [SMILES: *c1cccc2c1C(=O)N(C1CCC(=O)NC1=O)C2=O] |
| | | 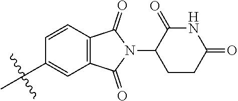 [SMILES: *c1ccc2c(c1)C(=O)N(C1CCC(=O)NC1=O)C2=O] |
| | 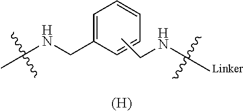 | 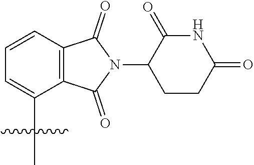 [SMILES: *c1cccc2c1C(=O)N(C1CCC(=O)NC1=O)C2=O] |
| | | 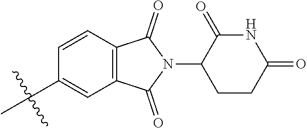 [SMILES: *c1ccc2c(c1)C(=O)N(C1CCC(=O)NC1=O)C2=O] |
| | 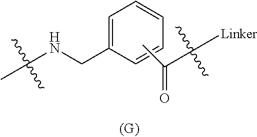 | 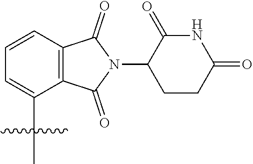 [SMILES: *c1cccc2c1C(=O)N(C1CCC(=O)NC1=O)C2=O] |
| | | 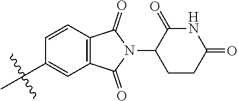 [SMILES: *c1ccc2c(c1)C(=O)N(C1CCC(=O)NC1=O)C2=O] |
| | 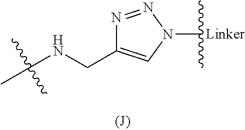 [SMILES: *NCc1cn(*)nn1] | 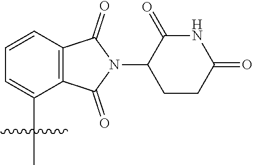 [SMILES: *c1cccc2c1C(=O)N(C1CCC(=O)NC1=O)C2=O] |
| | | 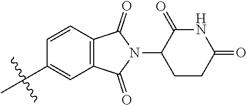 [SMILES: *c1ccc2c(c1)C(=O)N(C1CCC(=O)NC1=O)C2=O] |

Exemplary PROTAC compounds of this disclosure are illustrated in TABLE 2 and TABLE 3.

TABLE 2

| Compound | Structure |
|---|---|
| 8-4 | 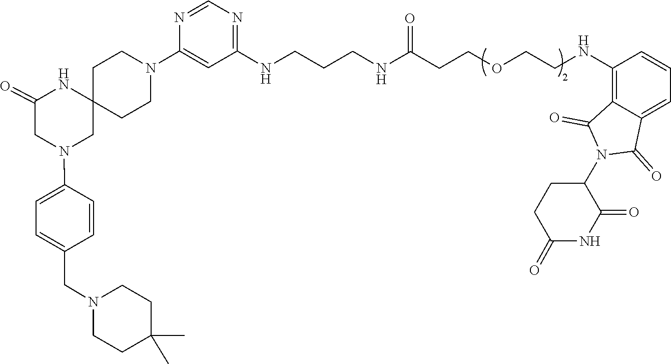 |
| | 8-4 |
| 9-5 | 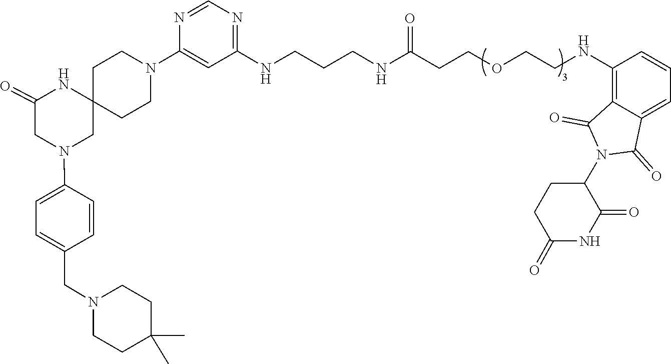 |
| | 9-5 |
| 10-6 | 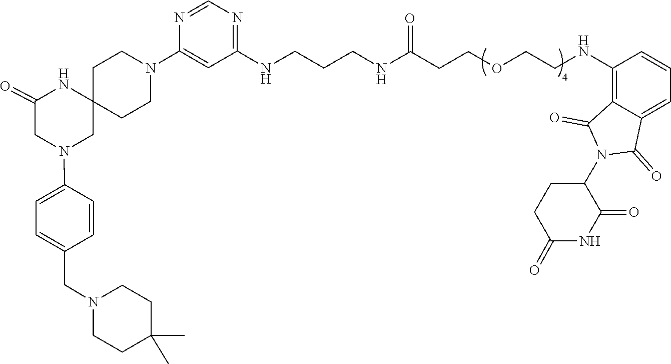 |
| | 10-6 |
| 11-7 | 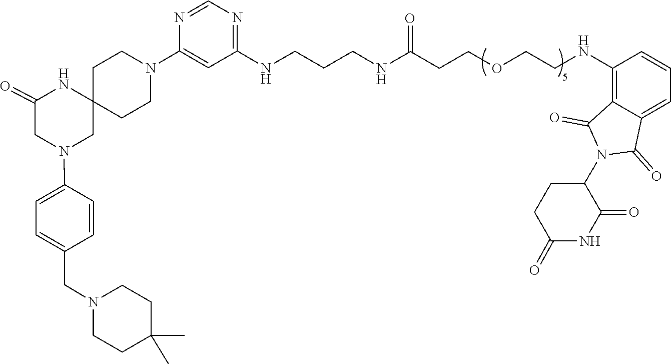 |
| | 11-7 |
| 13-11 | 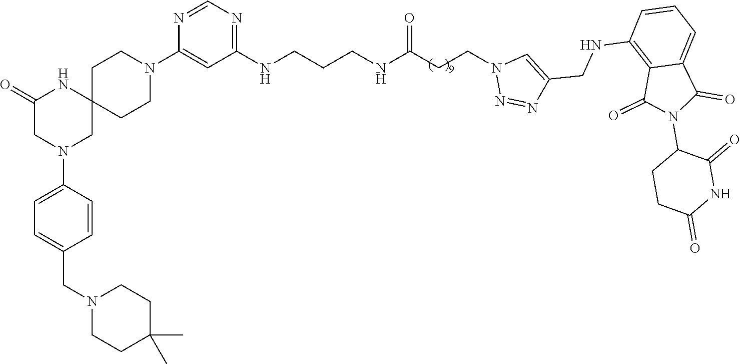 |
| | 13-11 |
| 14-12 | 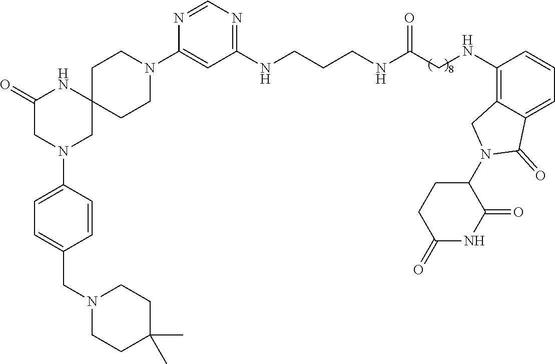 |
| | 14-12 |
| 15-13 | 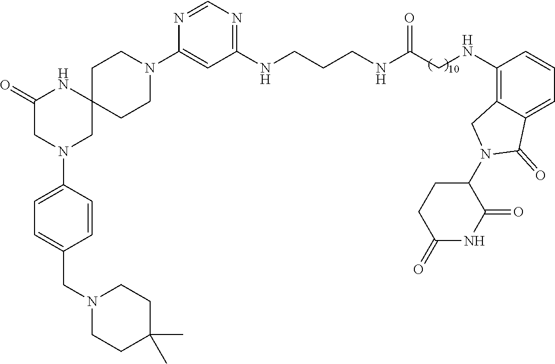 |
| | 15-13 |
| 49-1 | 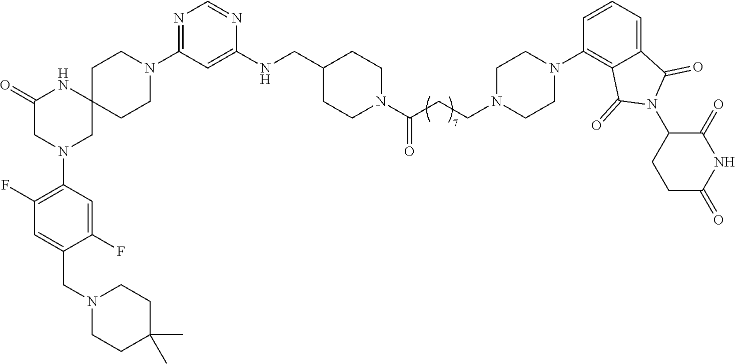 |
| | 49-1 |
| 50-1 | 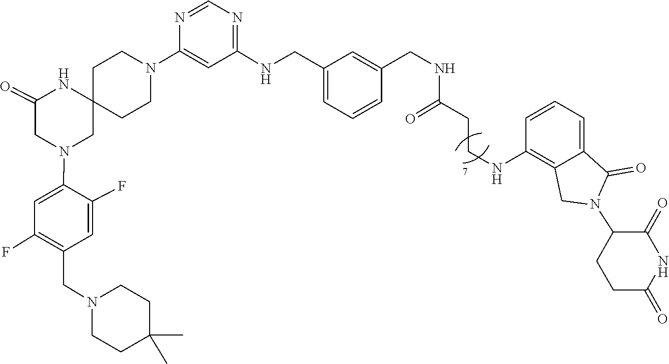 |
| | 50-1 |
| 51-1 | 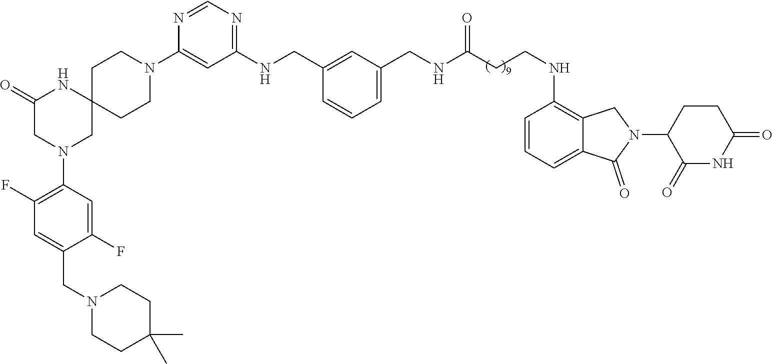 |
| | 51-1 |
| 52-1 | 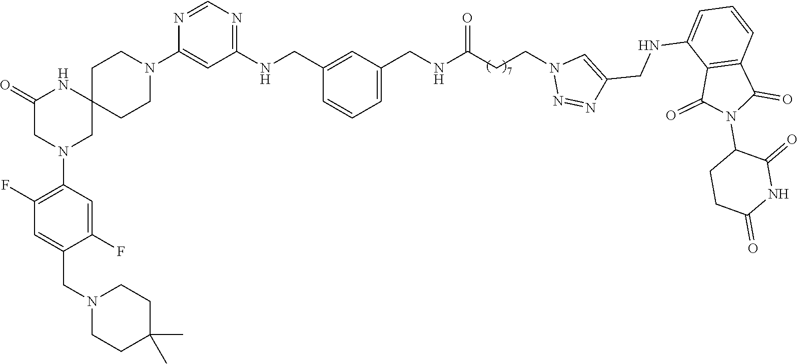 |
| | 52-1 |
| 53-1 | 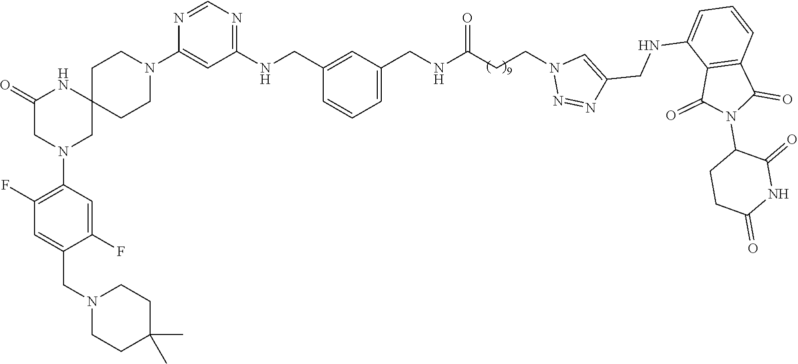 |
| | 53-1 |
| 54-1 | 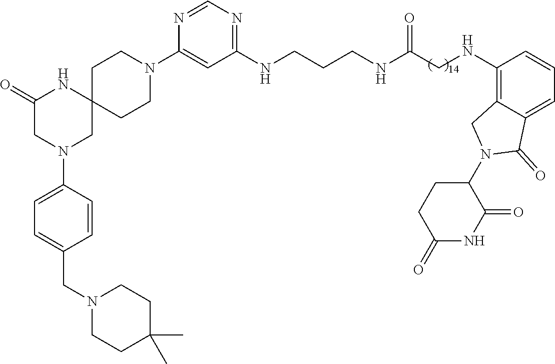 |
| | 54-1 |
| 55-1 | 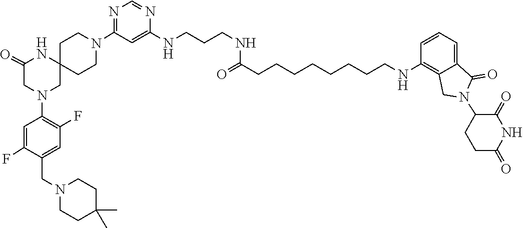 [SMILES: CC1(C)CCN(Cc2cc(F)c(N3CC(=O)NC4(CCN(c5cc(NCCCNC(=O)CCCCCCCCNc6cccc7c6CN(C6CCC(=O)NC6=O)C7=O)ncn5)CC4)C3)cc2F)CC1] |
| | 55-1 |
| 56-1 | 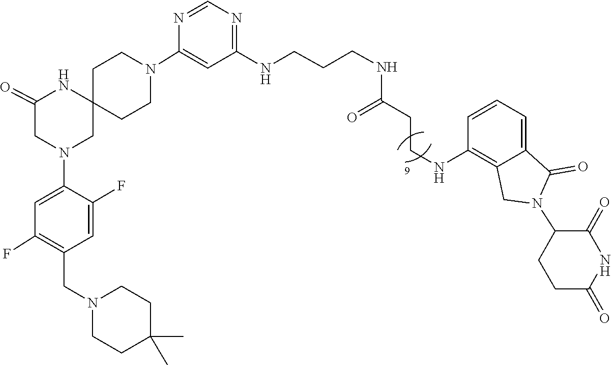 |
| | 56-1 |
| 57-1 | 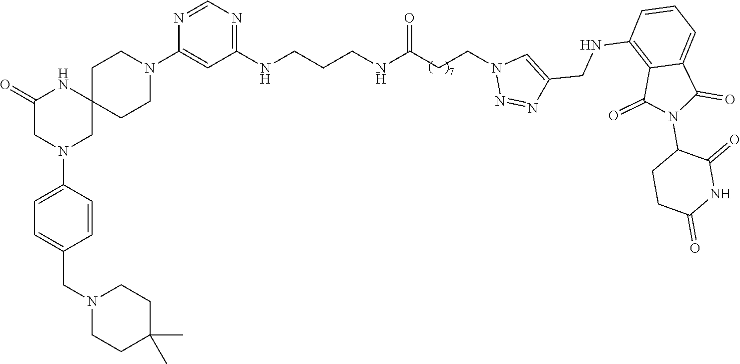 |
| | 57-1 |
| 58-1 | 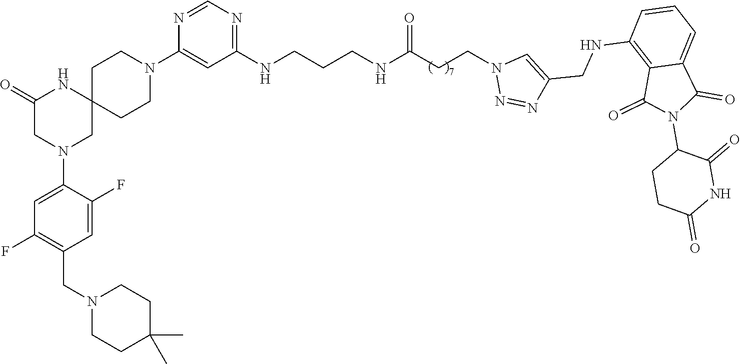 |
| | 58-1 |
| 59-1 | 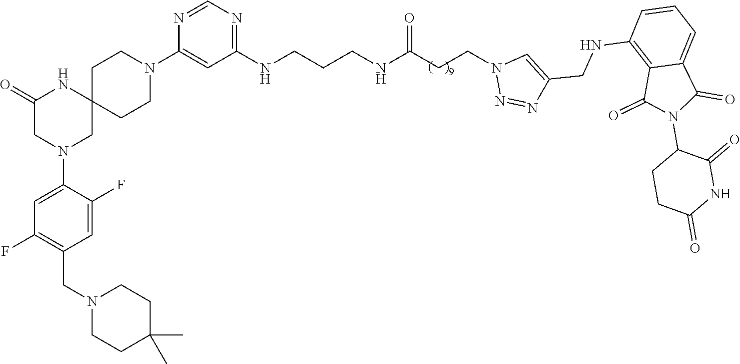 |
| | 59-1 |

TABLE 3

| Ex- | War- | | |
|---|---|---|---|
| am- | head | | |
| ple | ID | Warhead Handle Linker Recruiter | |
| 1 | 1 | 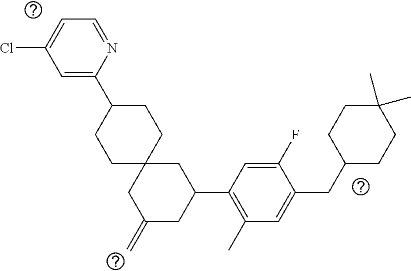 [SMILES: C=C1CC(c2cc(F)c(CC3CCC(C)(C)CC3)cc2C)CC2(CCC(c3cc(Cl)ccn3)CC2)C1] | 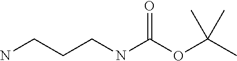 |
| | | 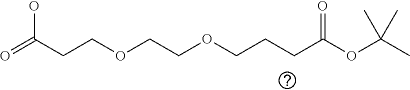 [SMILES: CC(C)(C)OC(=O)CCCOCCOCCC(=O)O] | 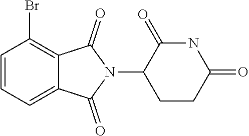 [SMILES: O=C1CCC(N2C(=O)c3cccc(Br)c3C2=O)C(=O)N1] |
| 2 | 1 | 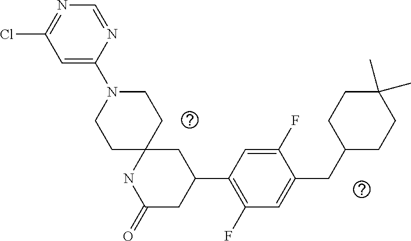 [SMILES: CC1(C)CCC(Cc2cc(F)c(C3CC(=O)NC4(CCN(c5cc(Cl)ncn5)CC4)C3)cc2F)CC1] | 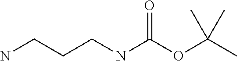 |
| | | 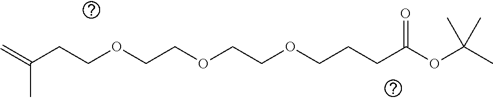 [SMILES: C=C(C)CCOCCOCCOCCCC(=O)OC(C)(C)C] | 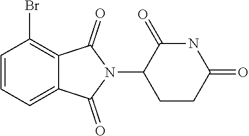 [SMILES: O=C1CCC(N2C(=O)c3cccc(Br)c3C2=O)C(=O)N1] |
| 3 | 1 | 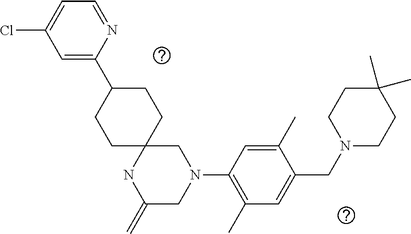 [SMILES: C=C1CN(c2cc(C)c(CN3CCC(C)(C)CC3)cc2C)CC2(CCC(c3cc(Cl)ccn3)CC2)N1] | 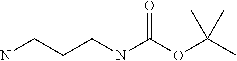 |
| | | 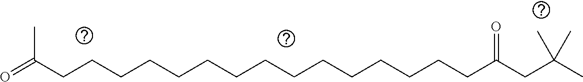 [SMILES: CC(=O)CCCCCCCCCCCCCCCC(=O)CC(C)(C)C] | 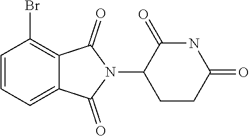 [SMILES: O=C1CCC(N2C(=O)c3cccc(Br)c3C2=O)C(=O)N1] |
| 4 | 1 | 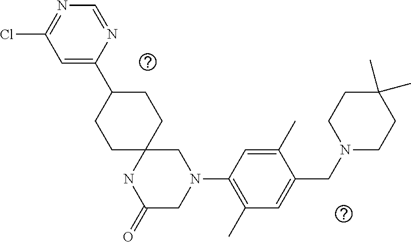 [SMILES: Cc1cc(N2CC(=O)NC3(CCC(c4cc(Cl)ncn4)CC3)C2)c(C)cc1CN1CCC(C)(C)CC1] | 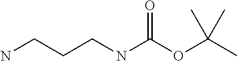 |
| | | 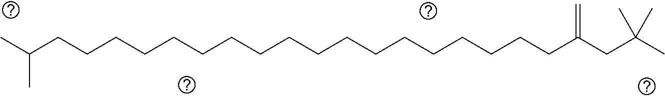 [SMILES: C=C(CCCCCCCCCCCCCCCCCCC(C)C)CC(C)(C)C] | 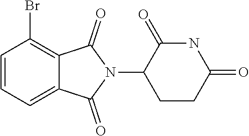 [SMILES: O=C1CCC(N2C(=O)c3cccc(Br)c3C2=O)C(=O)N1] |
| 5 | 1 | 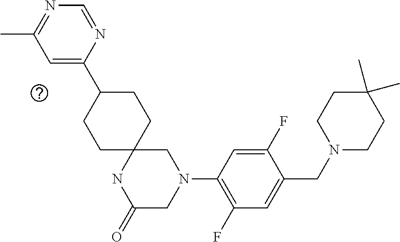 | 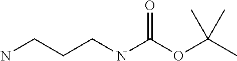 |
| | | 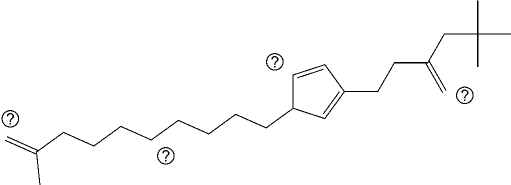 [SMILES: C=C(C)CCCCCCCCC1C=CC(CCC(=C)CC(C)(C)C)=C1] | 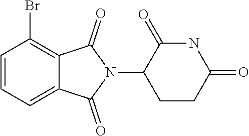 [SMILES: O=C1CCC(N2C(=O)c3cccc(Br)c3C2=O)C(=O)N1] |
| 6 | 1 | 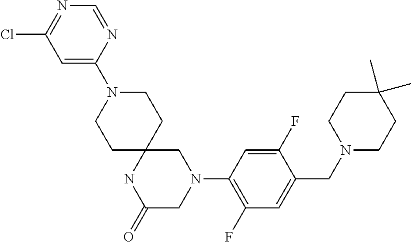 [SMILES: CC1(C)CCN(Cc2cc(F)c(N3CC(=O)NC4(CCN(c5cc(Cl)ncn5)CC4)C3)cc2F)CC1] | 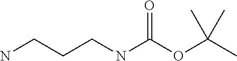 |
| | | 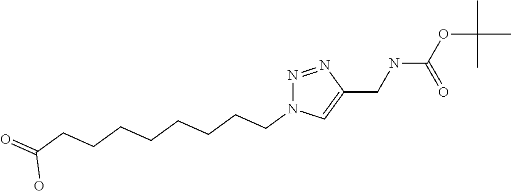 [SMILES: CC(C)(C)OC(=O)NCc1cn(CCCCCCCCC(=O)O)nn1] | 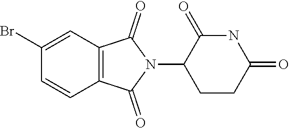 [SMILES: O=C1CCC(N2C(=O)c3ccc(Br)cc3C2=O)C(=O)N1] |
| 7 | 1 | 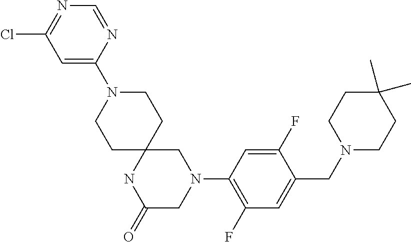 [SMILES: CC1(C)CCN(Cc2cc(F)c(N3CC(=O)NC4(CCN(c5cc(Cl)ncn5)CC4)C3)cc2F)CC1] | 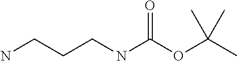 |
| | | 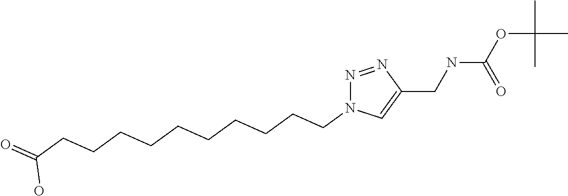 [SMILES: CC(C)(C)OC(=O)NCc1cn(CCCCCCCCCCC(=O)O)nn1] | 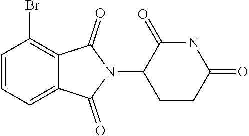 [SMILES: O=C1CCC(N2C(=O)c3cccc(Br)c3C2=O)C(=O)N1] |
| 8 | 1 | 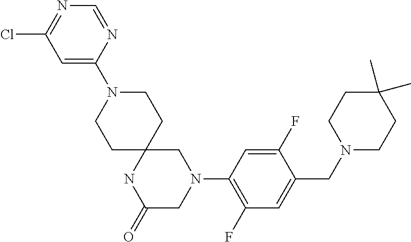 [SMILES: CC1(C)CCN(Cc2cc(F)c(N3CC(=O)NC4(CCN(c5cc(Cl)ncn5)CC4)C3)cc2F)CC1] | 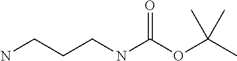 |
| | | 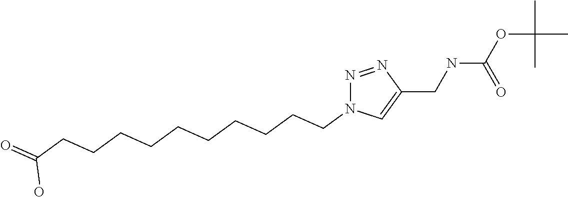 [SMILES: CC(C)(C)OC(=O)NCc1cn(CCCCCCCCCCC(=O)O)nn1] | 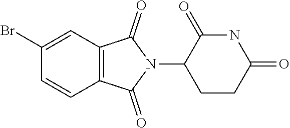 [SMILES: O=C1CCC(N2C(=O)c3ccc(Br)cc3C2=O)C(=O)N1] |
| 9 | 1 | 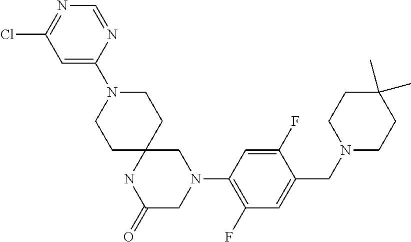 [SMILES: CC1(C)CCN(Cc2cc(F)c(N3CC(=O)NC4(CCN(c5cc(Cl)ncn5)CC4)C3)cc2F)CC1] | 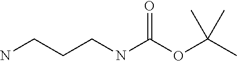 |
| | | 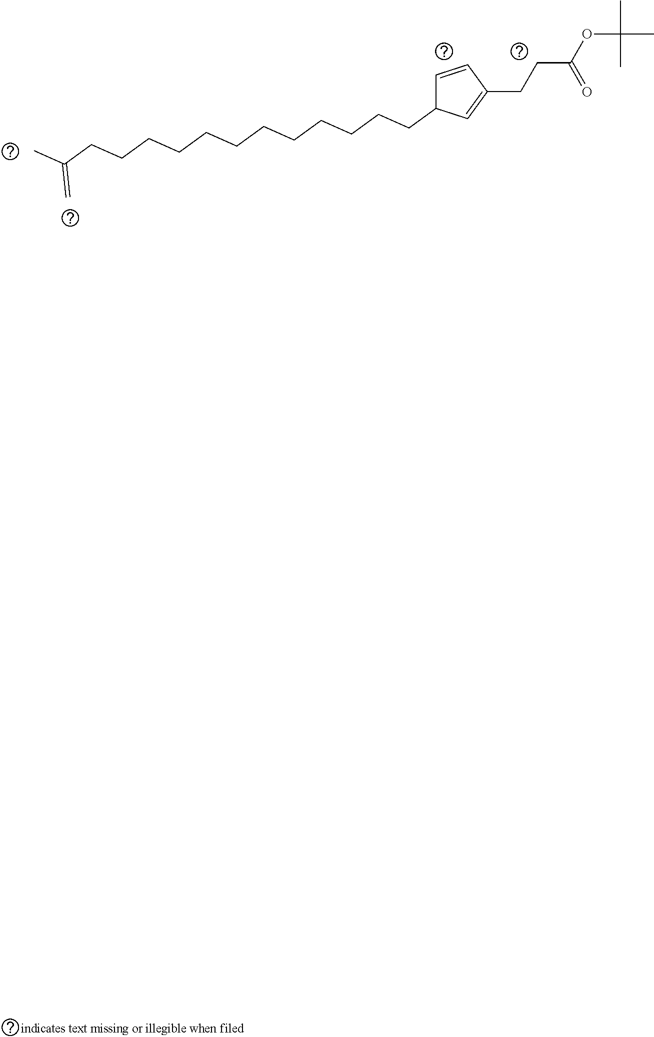 [SMILES: C=C(C)CCCCCCCCCCCCC1C=CC(CCC(=O)OC(C)(C)C)=C1] | 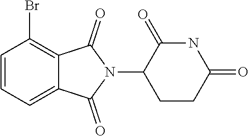 [SMILES: O=C1CCC(N2C(=O)c3cccc(Br)c3C2=O)C(=O)N1] |
| 10 | 1 | 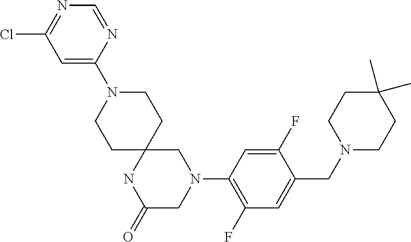 [SMILES: CC1(C)CCN(Cc2cc(F)c(N3CC(=O)NC4(CCN(c5cc(Cl)ncn5)CC4)C3)cc2F)CC1] | 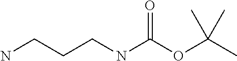 |
| | | 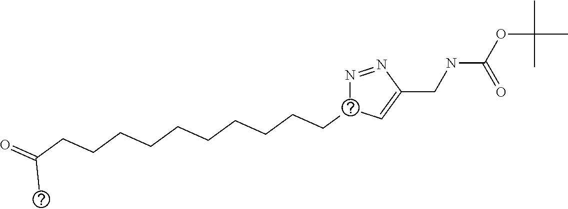 [SMILES: CC(=O)CCCCCCCCCCC1C=C(CNC(=O)OC(C)(C)C)N=N1] | 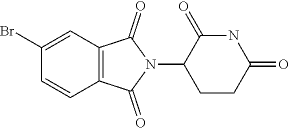 [SMILES: O=C1CCC(N2C(=O)c3ccc(Br)cc3C2=O)C(=O)N1] |
| 11 | 1 | 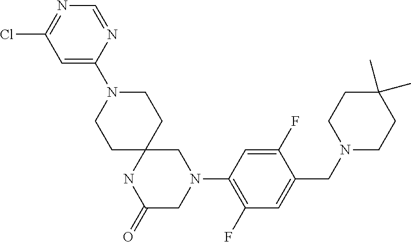 [SMILES: CC1(C)CCN(Cc2cc(F)c(N3CC(=O)NC4(CCN(c5cc(Cl)ncn5)CC4)C3)cc2F)CC1] | 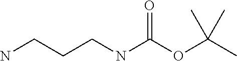 |
| | | 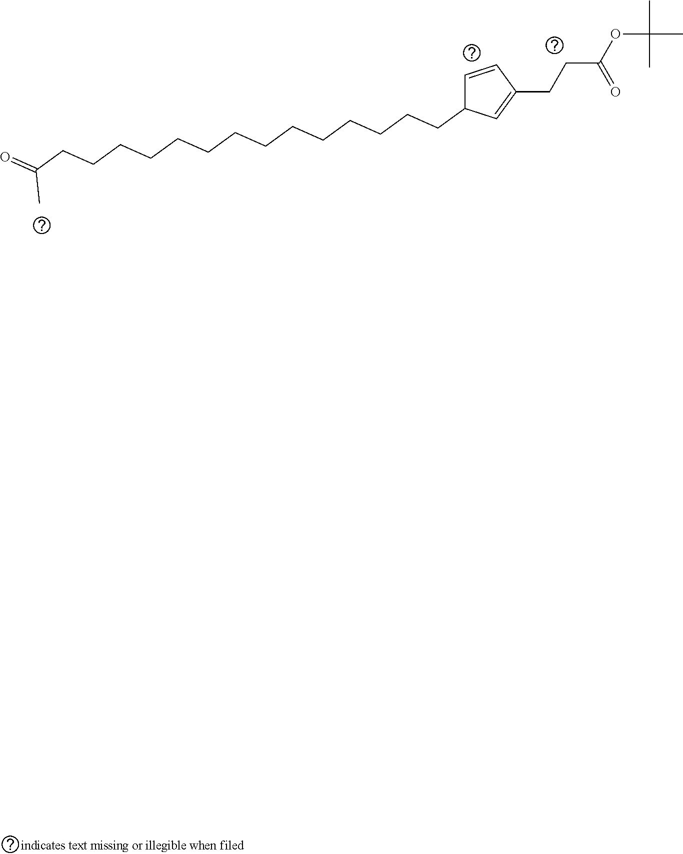 [SMILES: CC(=O)CCCCCCCCCCCCCCC1C=CC(CCC(=O)OC(C)(C)C)=C1] | 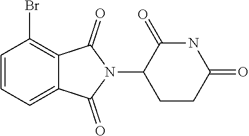 [SMILES: O=C1CCC(N2C(=O)c3cccc(Br)c3C2=O)C(=O)N1] |
| 12 | 1 | 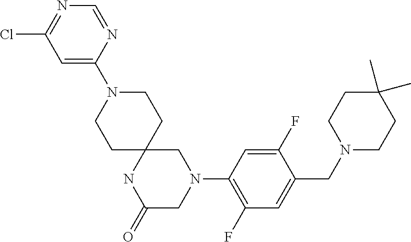 [SMILES: CC1(C)CCN(Cc2cc(F)c(N3CC(=O)NC4(CCN(c5cc(Cl)ncn5)CC4)C3)cc2F)CC1] | 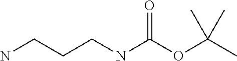 |
| | | 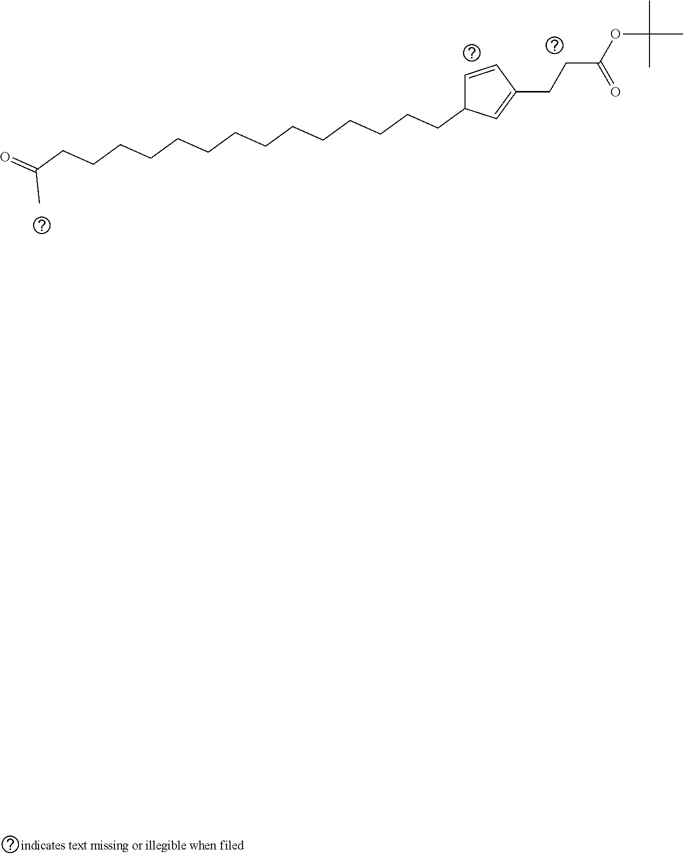 [SMILES: CC(=O)CCCCCCCCCCCCCCC1C=CC(CCC(=O)OC(C)(C)C)=C1] | 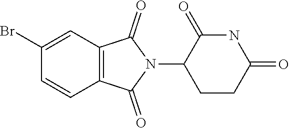 [SMILES: O=C1CCC(N2C(=O)c3ccc(Br)cc3C2=O)C(=O)N1] |
| 13 | 1 | 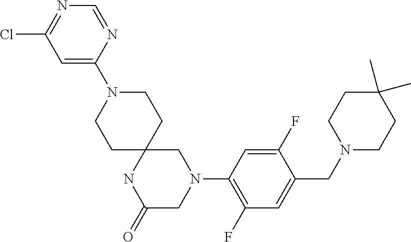 [SMILES: CC1(C)CCN(Cc2cc(F)c(N3CC(=O)NC4(CCN(c5cc(Cl)ncn5)CC4)C3)cc2F)CC1] | 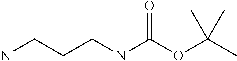 |
| | | 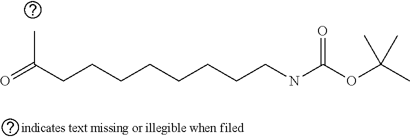 [SMILES: CC(=O)CCCCCCCCNC(=O)OC(C)(C)C] | 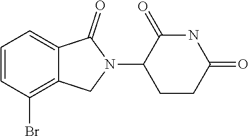 [SMILES: O=C1CCC(N2Cc3c(Br)cccc3C2=O)C(=O)N1] |
| 14 | 1 | 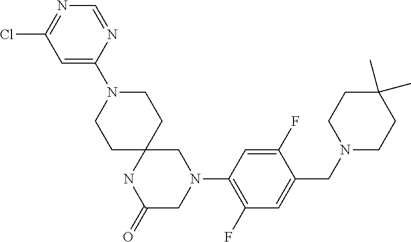 [SMILES: CC1(C)CCN(Cc2cc(F)c(N3CC(=O)NC4(CCN(c5cc(Cl)ncn5)CC4)C3)cc2F)CC1] | 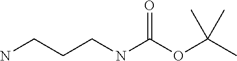 |
| | | 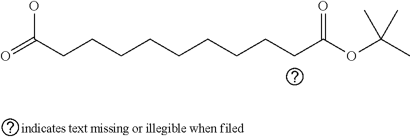 [SMILES: CC(C)(C)OC(=O)CCCCCCCCCC(=O)O] | 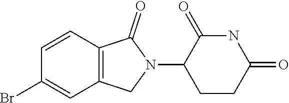 [SMILES: O=C1CCC(N2Cc3cc(Br)ccc3C2=O)C(=O)N1] |
| 15 | 1 | 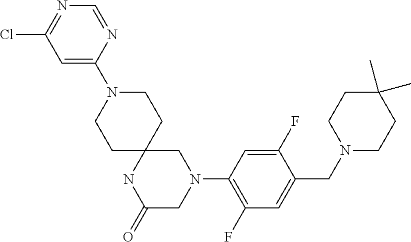 [SMILES: CC1(C)CCN(Cc2cc(F)c(N3CC(=O)NC4(CCN(c5cc(Cl)ncn5)CC4)C3)cc2F)CC1] | 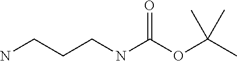 |
| | | 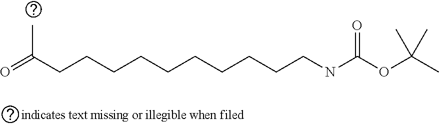 [SMILES: CC(=O)CCCCCCCCCCNC(=O)OC(C)(C)C] | 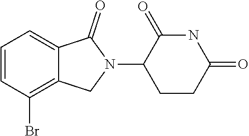 [SMILES: O=C1CCC(N2Cc3c(Br)cccc3C2=O)C(=O)N1] |
| 16 | 1 | 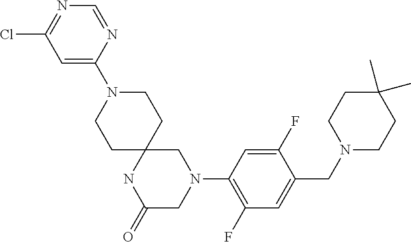 [SMILES: CC1(C)CCN(Cc2cc(F)c(N3CC(=O)NC4(CCN(c5cc(Cl)ncn5)CC4)C3)cc2F)CC1] | 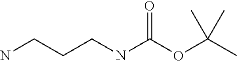 |
| | | 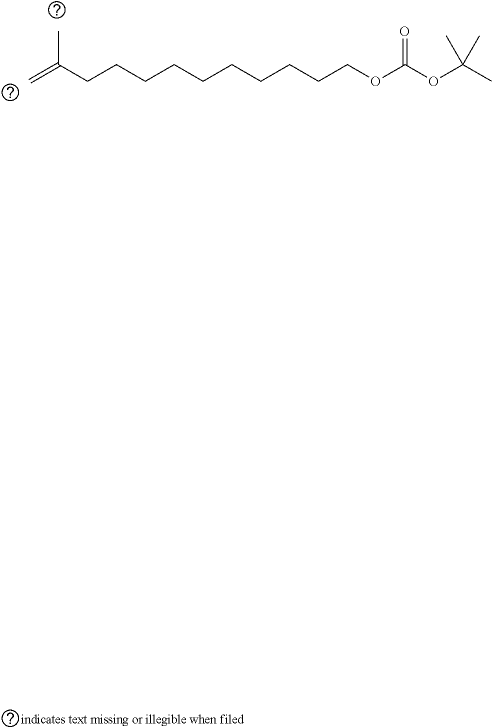 [SMILES: C=C(C)CCCCCCCCCCOC(=O)OC(C)(C)C] | 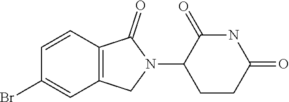 [SMILES: O=C1CCC(N2Cc3cc(Br)ccc3C2=O)C(=O)N1] |
| 17 | 1 | 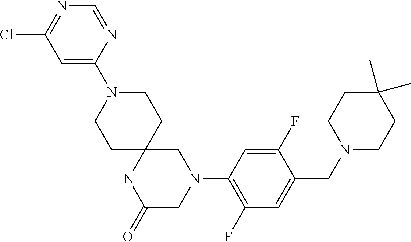 [SMILES: CC1(C)CCN(Cc2cc(F)c(N3CC(=O)NC4(CCN(c5cc(Cl)ncn5)CC4)C3)cc2F)CC1] | 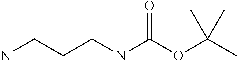 |
| | | 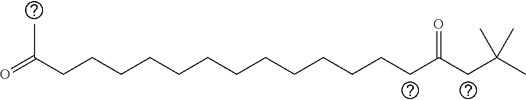 [SMILES: CC(=O)CCCCCCCCCCCCCC(=O)CC(C)(C)C] | 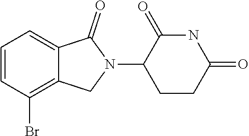 [SMILES: O=C1CCC(N2Cc3c(Br)cccc3C2=O)C(=O)N1] |
| 18 | 1 | 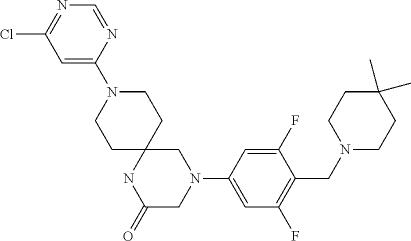 [SMILES: CC1(C)CCN(Cc2c(F)cc(N3CC(=O)NC4(CCN(c5cc(Cl)ncn5)CC4)C3)cc2F)CC1] | 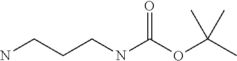 |
| | | 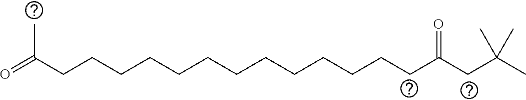 [SMILES: CC(=O)CCCCCCCCCCCCCC(=O)CC(C)(C)C] | 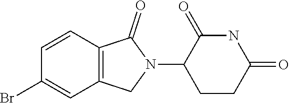 [SMILES: O=C1CCC(N2Cc3cc(Br)ccc3C2=O)C(=O)N1] |
| 19 | 1 | 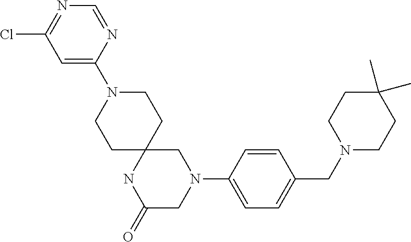 [SMILES: CC1(C)CCN(Cc2ccc(N3CC(=O)NC4(CCN(c5cc(Cl)ncn5)CC4)C3)cc2)CC1] | 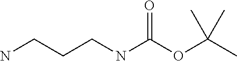 |
| | | 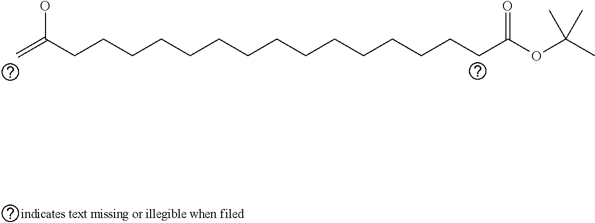 [SMILES: C=C(O)CCCCCCCCCCCCCCCC(=O)OC(C)(C)C] | 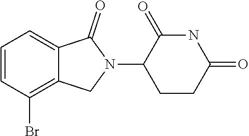 [SMILES: O=C1CCC(N2Cc3c(Br)cccc3C2=O)C(=O)N1] |
| 20 | 1 | 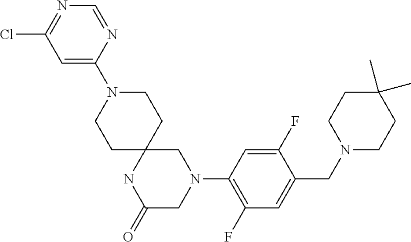 [SMILES: CC1(C)CCN(Cc2cc(F)c(N3CC(=O)NC4(CCN(c5cc(Cl)ncn5)CC4)C3)cc2F)CC1] | 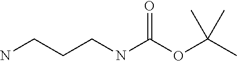 |
| | | 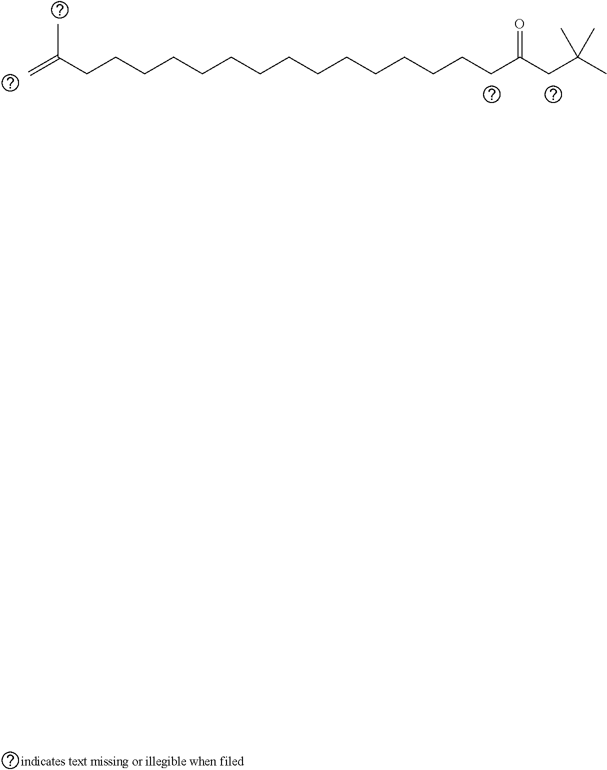 [SMILES: C=C(C)CCCCCCCCCCCCCCCC(=O)CC(C)(C)C] | 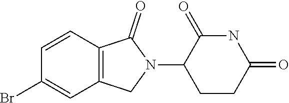 [SMILES: O=C1CCC(N2Cc3cc(Br)ccc3C2=O)C(=O)N1] |
| 21 | 1 | 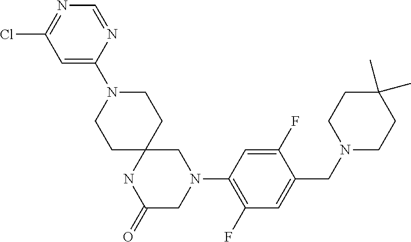 [SMILES: CC1(C)CCN(Cc2cc(F)c(N3CC(=O)NC4(CCN(c5cc(Cl)ncn5)CC4)C3)cc2F)CC1] | 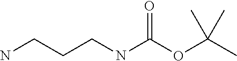 |
| | | 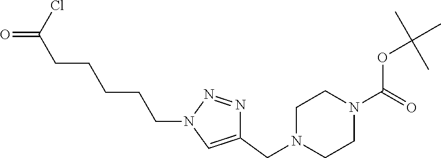 [SMILES: CC(C)(C)OC(=O)N1CCN(Cc2cn(CCCCCC(=O)Cl)nn2)CC1] | 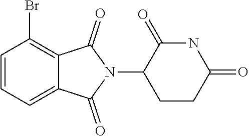 [SMILES: O=C1CCC(N2C(=O)c3cccc(Br)c3C2=O)C(=O)N1] |
| 22 | 1 | 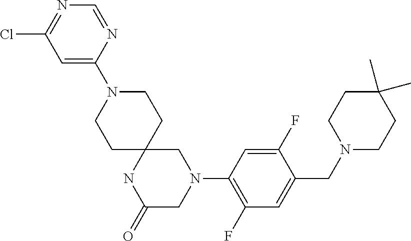 [SMILES: CC1(C)CCN(Cc2cc(F)c(N3CC(=O)NC4(CCN(c5cc(Cl)ncn5)CC4)C3)cc2F)CC1] | 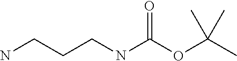 |
| | | 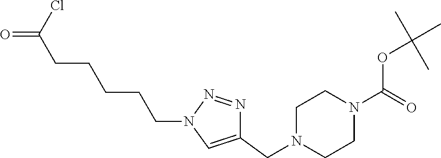 [SMILES: CC(C)(C)OC(=O)N1CCN(Cc2cn(CCCCCC(=O)Cl)nn2)CC1] | 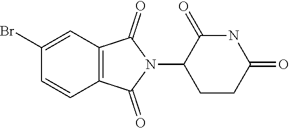 [SMILES: O=C1CCC(N2C(=O)c3ccc(Br)cc3C2=O)C(=O)N1] |
| 23 | 1 | 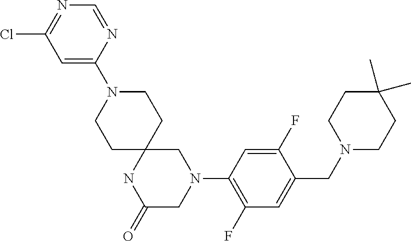 [SMILES: CC1(C)CCN(Cc2cc(F)c(N3CC(=O)NC4(CCN(c5cc(Cl)ncn5)CC4)C3)cc2F)CC1] | 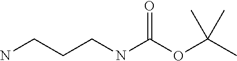 |
| | | 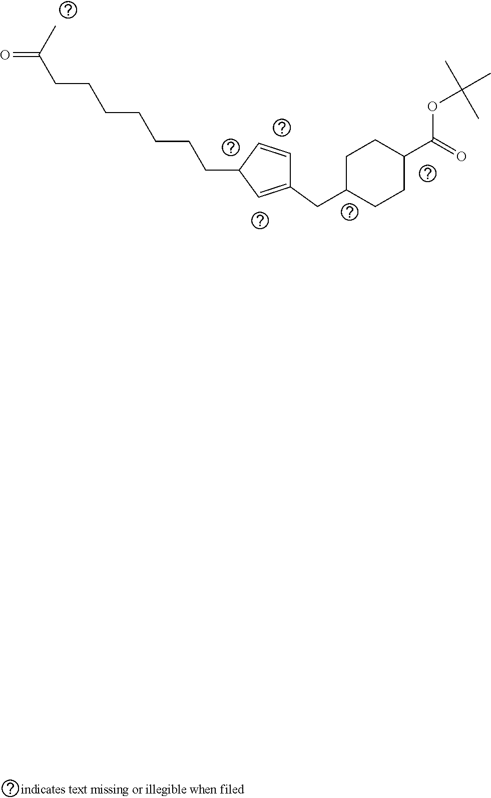 [SMILES: CC(=O)CCCCCCCC1C=CC(CC2CCC(C(=O)OC(C)(C)C)CC2)=C1] | 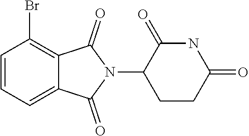 [SMILES: O=C1CCC(N2C(=O)c3cccc(Br)c3C2=O)C(=O)N1] |
| 24 | 1 | 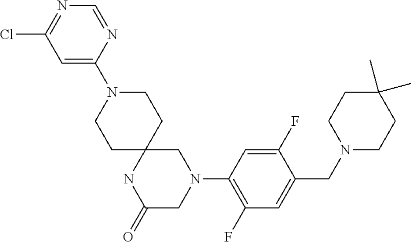 [SMILES: CC1(C)CCN(Cc2cc(F)c(N3CC(=O)NC4(CCN(c5cc(Cl)ncn5)CC4)C3)cc2F)CC1] | 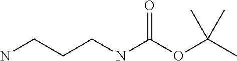 |
| | | 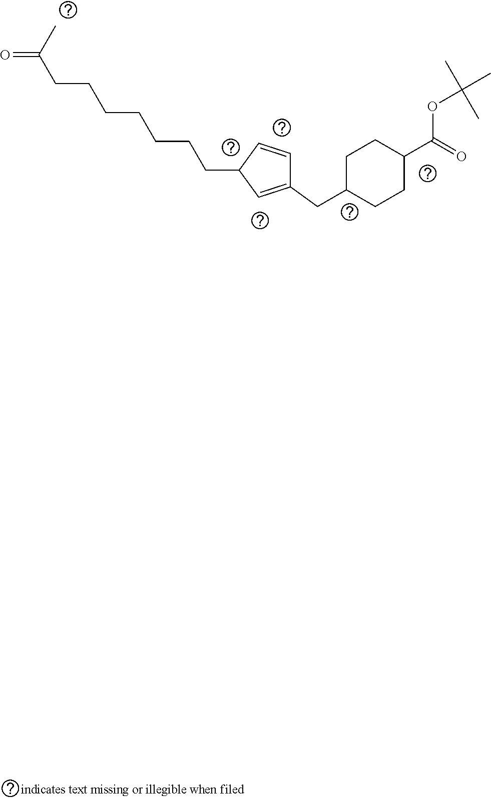 [SMILES: CC(=O)CCCCCCCC1C=CC(CC2CCC(C(=O)OC(C)(C)C)CC2)=C1] | 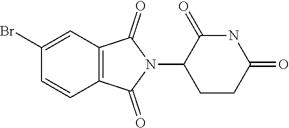 [SMILES: O=C1CCC(N2C(=O)c3ccc(Br)cc3C2=O)C(=O)N1] |
| 25 | 1 | 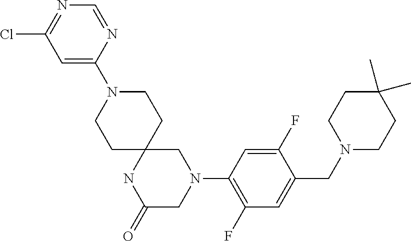 [SMILES: CC1(C)CCN(Cc2cc(F)c(N3CC(=O)NC4(CCN(c5cc(Cl)ncn5)CC4)C3)cc2F)CC1] | 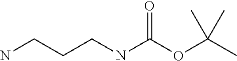 |
| | | 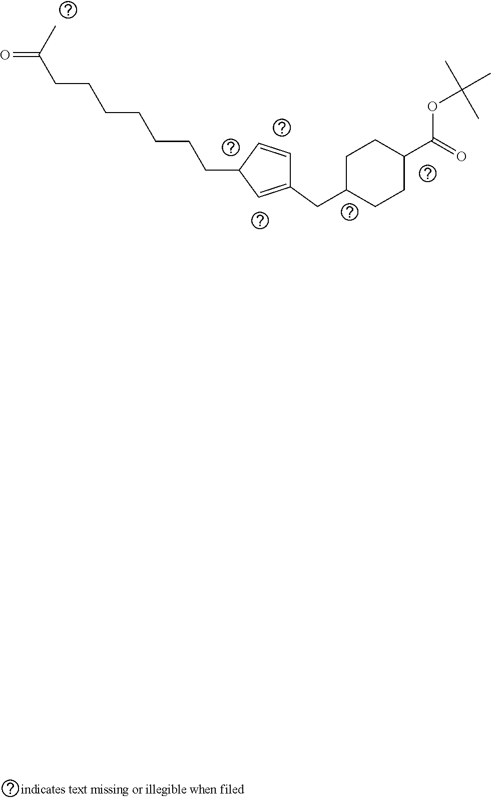 [SMILES: CC(=O)CCCCCCCC1C=CC(CC2CCC(C(=O)OC(C)(C)C)CC2)=C1] | 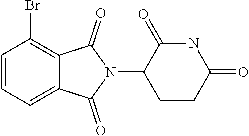 [SMILES: O=C1CCC(N2C(=O)c3cccc(Br)c3C2=O)C(=O)N1] |
| 26 | 1 | 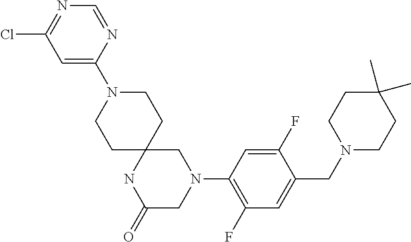 [SMILES: CC1(C)CCN(Cc2cc(F)c(N3CC(=O)NC4(CCN(c5cc(Cl)ncn5)CC4)C3)cc2F)CC1] | 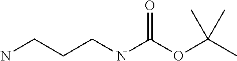 |
| | | 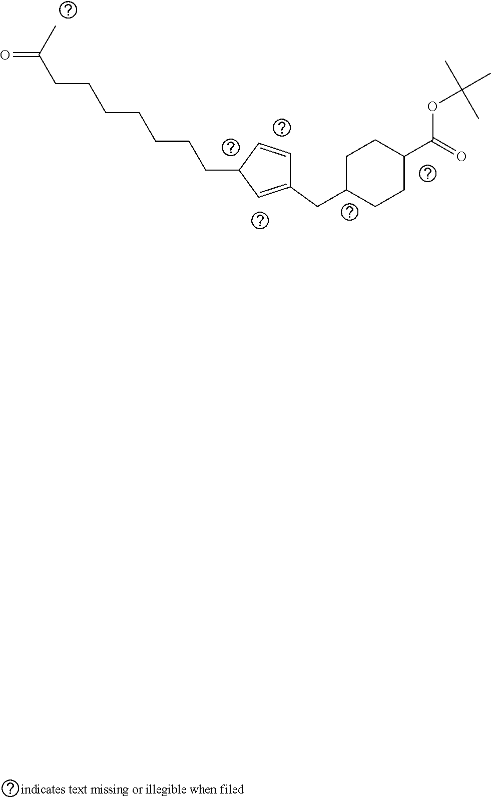 [SMILES: CC(=O)CCCCCCCC1C=CC(CC2CCC(C(=O)OC(C)(C)C)CC2)=C1] | 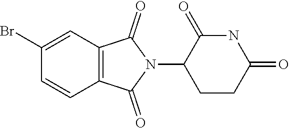 [SMILES: O=C1CCC(N2C(=O)c3ccc(Br)cc3C2=O)C(=O)N1] |
| 27 | 1 | 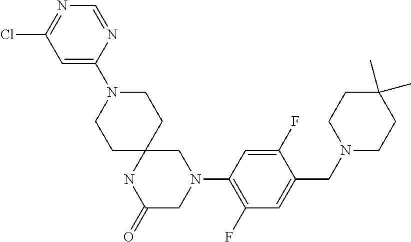 [SMILES: CC1(C)CCN(Cc2cc(F)c(N3CC(=O)NC4(CCN(c5cc(Cl)ncn5)CC4)C3)cc2F)CC1] | 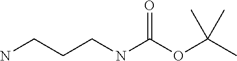 |
| | | 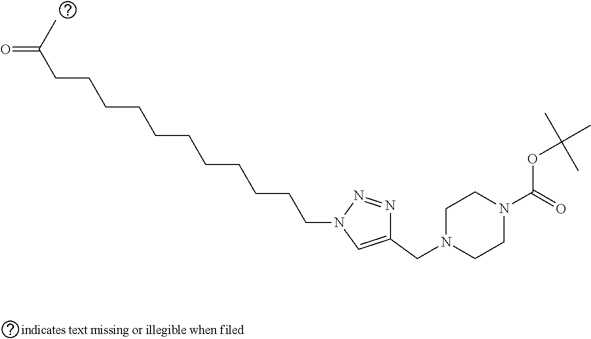 [SMILES: CC(=O)CCCCCCCCCCCn1cc(CN2CCN(C(=O)OC(C)(C)C)CC2)nn1] | 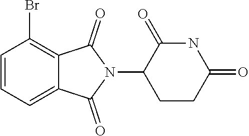 [SMILES: O=C1CCC(N2C(=O)c3cccc(Br)c3C2=O)C(=O)N1] |
| 28 | 1 | 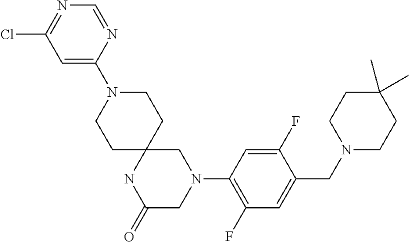 [SMILES: CC1(C)CCN(Cc2cc(F)c(N3CC(=O)NC4(CCN(c5cc(Cl)ncn5)CC4)C3)cc2F)CC1] | 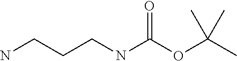 |
| | | 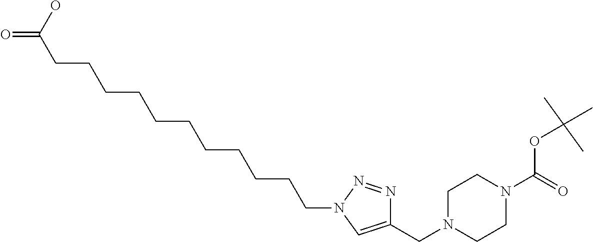 [SMILES: CC(C)(C)OC(=O)N1CCN(Cc2cn(CCCCCCCCCCCC(=O)O)nn2)CC1] | 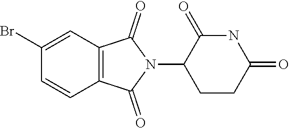 [SMILES: O=C1CCC(N2C(=O)c3ccc(Br)cc3C2=O)C(=O)N1] |
| 29 | 1 | 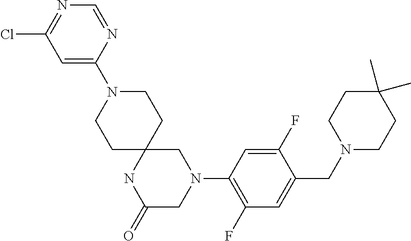 [SMILES: CC1(C)CCN(Cc2cc(F)c(N3CC(=O)NC4(CCN(c5cc(Cl)ncn5)CC4)C3)cc2F)CC1] | 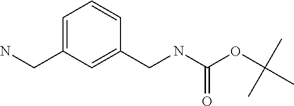 [SMILES: CC(C)(C)OC(=O)NCc1cccc(CN)c1] |
| | | 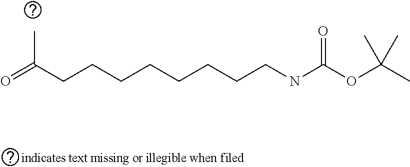 [SMILES: CC(=O)CCCCCCCCNC(=O)OC(C)(C)C] | 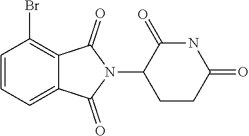 [SMILES: O=C1CCC(N2C(=O)c3cccc(Br)c3C2=O)C(=O)N1] |
| 30 | 1 | 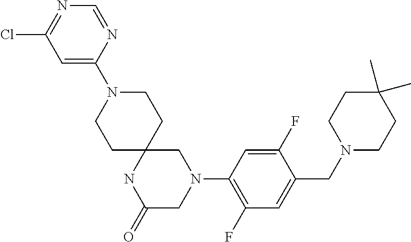 [SMILES: CC1(C)CCN(Cc2cc(F)c(N3CC(=O)NC4(CCN(c5cc(Cl)ncn5)CC4)C3)cc2F)CC1] | 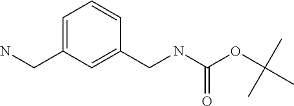 [SMILES: CC(C)(C)OC(=O)NCc1cccc(CN)c1] |
| | | 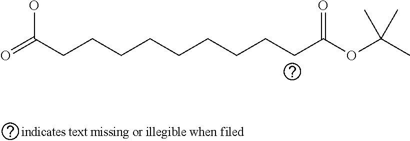 [SMILES: CC(C)(C)OC(=O)CCCCCCCCCC(=O)O] | 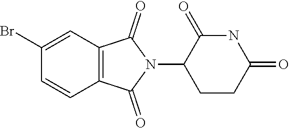 [SMILES: O=C1CCC(N2C(=O)c3ccc(Br)cc3C2=O)C(=O)N1] |
| 31 | 1 | 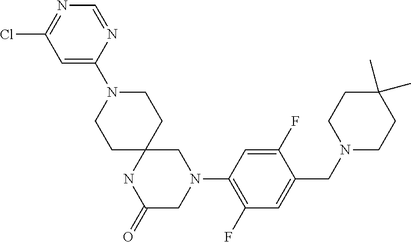 [SMILES: CC1(C)CCN(Cc2cc(F)c(N3CC(=O)NC4(CCN(c5cc(Cl)ncn5)CC4)C3)cc2F)CC1] | 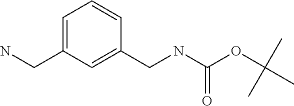 [SMILES: CC(C)(C)OC(=O)NCc1cccc(CN)c1] |
| | | 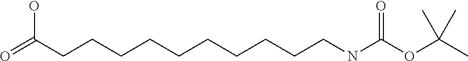 [SMILES: CC(C)(C)OC(=O)NCCCCCCCCCCC(=O)O] | 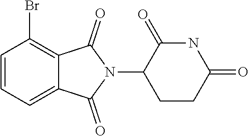 [SMILES: O=C1CCC(N2C(=O)c3cccc(Br)c3C2=O)C(=O)N1] |
| 32 | 1 | 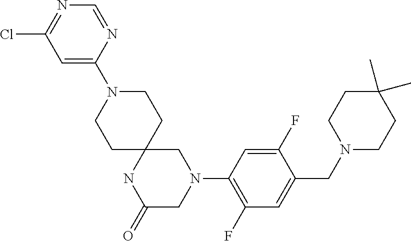 [SMILES: CC1(C)CCN(Cc2cc(F)c(N3CC(=O)NC4(CCN(c5cc(Cl)ncn5)CC4)C3)cc2F)CC1] | 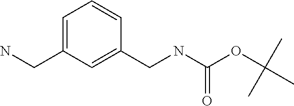 [SMILES: CC(C)(C)OC(=O)NCc1cccc(CN)c1] |
| | | 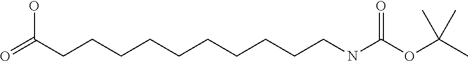 [SMILES: CC(C)(C)OC(=O)NCCCCCCCCCCC(=O)O] | 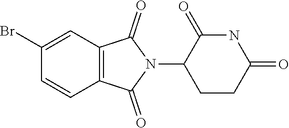 [SMILES: O=C1CCC(N2C(=O)c3ccc(Br)cc3C2=O)C(=O)N1] |
| 33 | 1 | 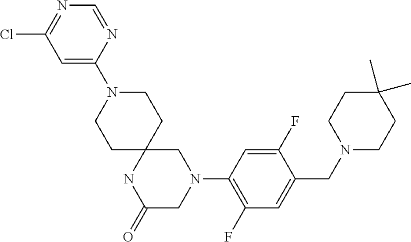 [SMILES: CC1(C)CCN(Cc2cc(F)c(N3CC(=O)NC4(CCN(c5cc(Cl)ncn5)CC4)C3)cc2F)CC1] | 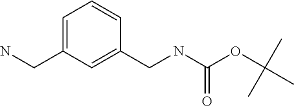 [SMILES: CC(C)(C)OC(=O)NCc1cccc(CN)c1] |
| | | 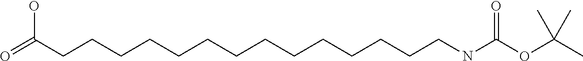 [SMILES: CC(C)(C)OC(=O)NCCCCCCCCCCCCCCC(=O)O] | 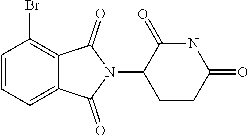 [SMILES: O=C1CCC(N2C(=O)c3cccc(Br)c3C2=O)C(=O)N1] |
| 34 | 1 | 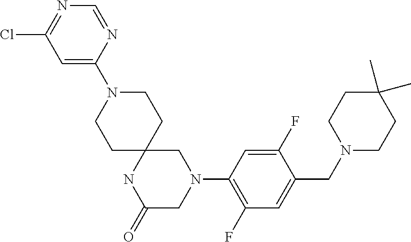 [SMILES: CC1(C)CCN(Cc2cc(F)c(N3CC(=O)NC4(CCN(c5cc(Cl)ncn5)CC4)C3)cc2F)CC1] | 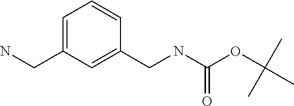 [SMILES: CC(C)(C)OC(=O)NCc1cccc(CN)c1] |
| | | 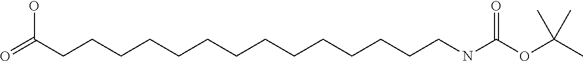 [SMILES: CC(C)(C)OC(=O)NCCCCCCCCCCCCCCC(=O)O] | 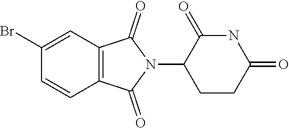 [SMILES: O=C1CCC(N2C(=O)c3ccc(Br)cc3C2=O)C(=O)N1] |
| 35 | 1 | 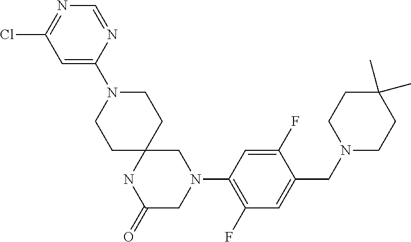 [SMILES: CC1(C)CCN(Cc2cc(F)c(N3CC(=O)NC4(CCN(c5cc(Cl)ncn5)CC4)C3)cc2F)CC1] | 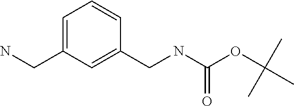 [SMILES: CC(C)(C)OC(=O)NCc1cccc(CN)c1] |
| | | 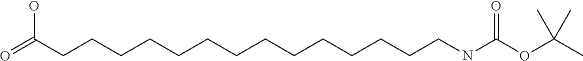 [SMILES: CC(C)(C)OC(=O)NCCCCCCCCCCCCCCC(=O)O] | 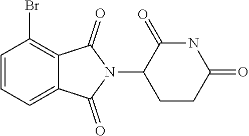 [SMILES: O=C1CCC(N2C(=O)c3cccc(Br)c3C2=O)C(=O)N1] |
| 36 | 1 | 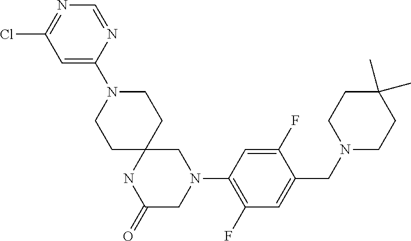 [SMILES: CC1(C)CCN(Cc2cc(F)c(N3CC(=O)NC4(CCN(c5cc(Cl)ncn5)CC4)C3)cc2F)CC1] | 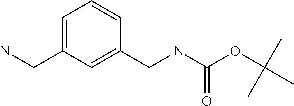 [SMILES: CC(C)(C)OC(=O)NCc1cccc(CN)c1] |
| | | 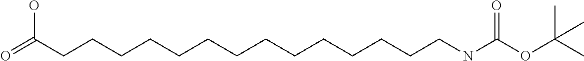 [SMILES: CC(C)(C)OC(=O)NCCCCCCCCCCCCCCC(=O)O] | 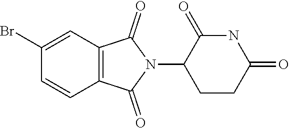 [SMILES: O=C1CCC(N2C(=O)c3ccc(Br)cc3C2=O)C(=O)N1] |
| 37 | 1 | 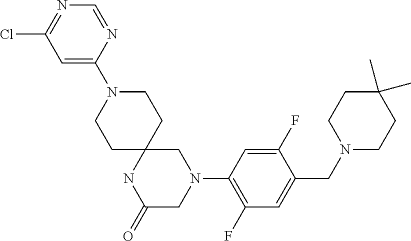 [SMILES: CC1(C)CCN(Cc2cc(F)c(N3CC(=O)NC4(CCN(c5cc(Cl)ncn5)CC4)C3)cc2F)CC1] | 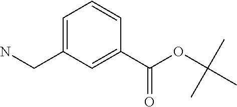 |
| | | 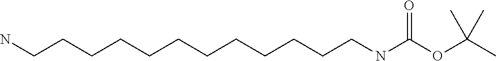 [SMILES: CC(C)(C)OC(=O)NCCCCCCCCCCCCN] | 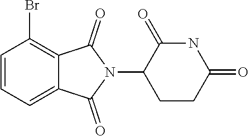 [SMILES: O=C1CCC(N2C(=O)c3cccc(Br)c3C2=O)C(=O)N1] |
| 38 | 1 | 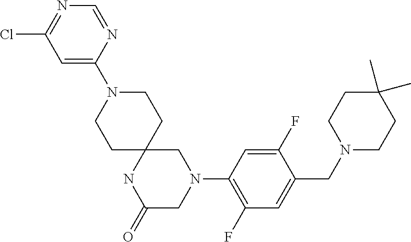 [SMILES: CC1(C)CCN(Cc2cc(F)c(N3CC(=O)NC4(CCN(c5cc(Cl)ncn5)CC4)C3)cc2F)CC1] | 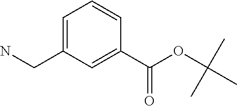 |
| | | 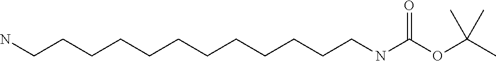 [SMILES: CC(C)(C)OC(=O)NCCCCCCCCCCCCN] | 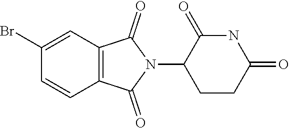 [SMILES: O=C1CCC(N2C(=O)c3ccc(Br)cc3C2=O)C(=O)N1] |
| 39 | 1 | 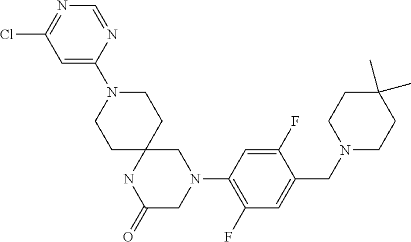 [SMILES: CC1(C)CCN(Cc2cc(F)c(N3CC(=O)NC4(CCN(c5cc(Cl)ncn5)CC4)C3)cc2F)CC1] | 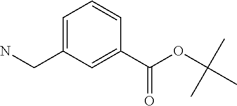 |
| | | 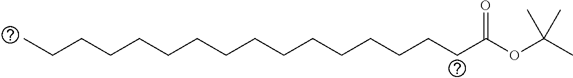 [SMILES: CCCCCCCCCCCCCCCCC(=O)OC(C)(C)C] | 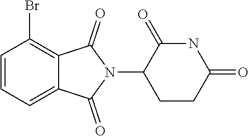 [SMILES: O=C1CCC(N2C(=O)c3cccc(Br)c3C2=O)C(=O)N1] |
| 40 | 1 | 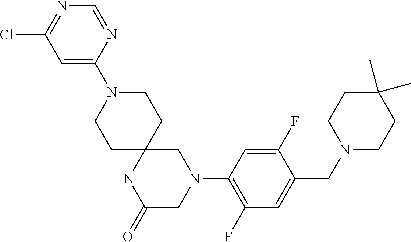 [SMILES: CC1(C)CCN(Cc2cc(F)c(N3CC(=O)NC4(CCN(c5cc(Cl)ncn5)CC4)C3)cc2F)CC1] | 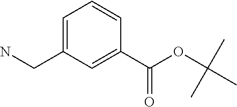 |
| | | 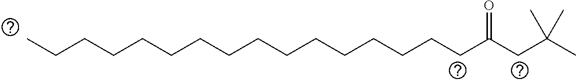 [SMILES: CCCCCCCCCCCCCCCCC(=O)CC(C)(C)C] | 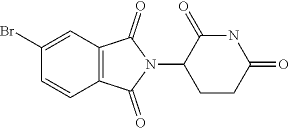 [SMILES: O=C1CCC(N2C(=O)c3ccc(Br)cc3C2=O)C(=O)N1] |
| 41 | 1 | 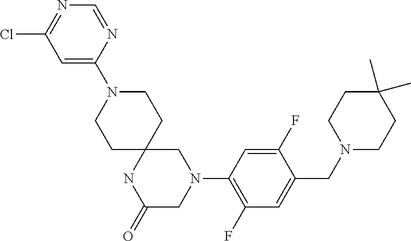 [SMILES: CC1(C)CCN(Cc2cc(F)c(N3CC(=O)NC4(CCN(c5cc(Cl)ncn5)CC4)C3)cc2F)CC1] | 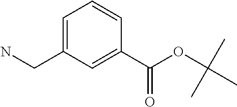 |
| | | 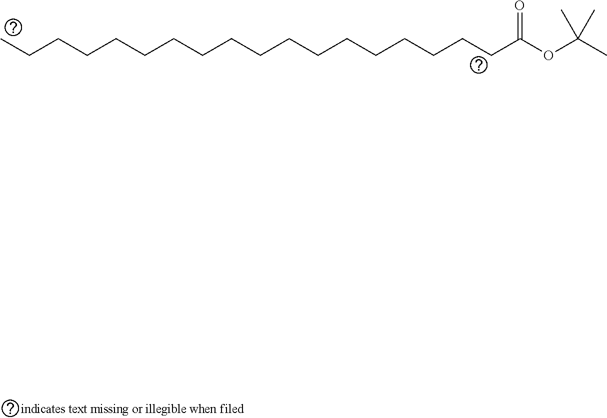 [SMILES: CCCCCCCCCCCCCCCCCCC(=O)OC(C)(C)C] | 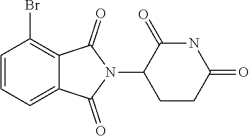 [SMILES: O=C1CCC(N2C(=O)c3cccc(Br)c3C2=O)C(=O)N1] |
| 42 | 1 | 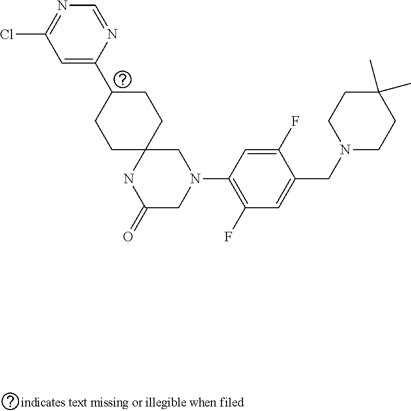 [SMILES: CC1(C)CCN(Cc2cc(F)c(N3CC(=O)NC4(CCC(c5cc(Cl)ncn5)CC4)C3)cc2F)CC1] | 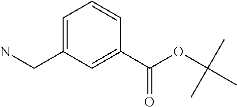 |
| | | 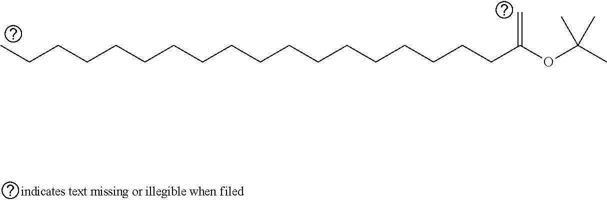 [SMILES: C=C(CCCCCCCCCCCCCCCCCC)OC(C)(C)C] | 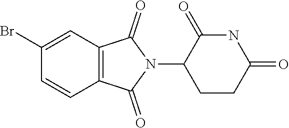 [SMILES: O=C1CCC(N2C(=O)c3ccc(Br)cc3C2=O)C(=O)N1] |
| 43 | 1 | 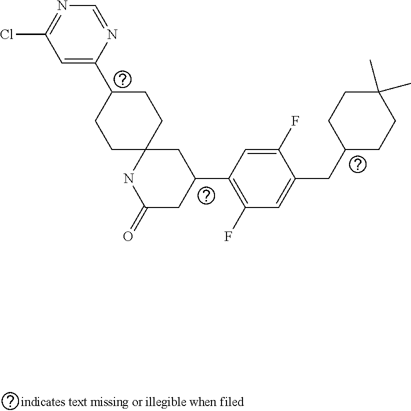 [SMILES: CC1(C)CCC(Cc2cc(F)c(C3CC(=O)NC4(CCC(c5cc(Cl)ncn5)CC4)C3)cc2F)CC1] | 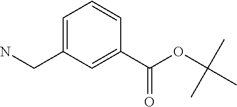 |
| | | 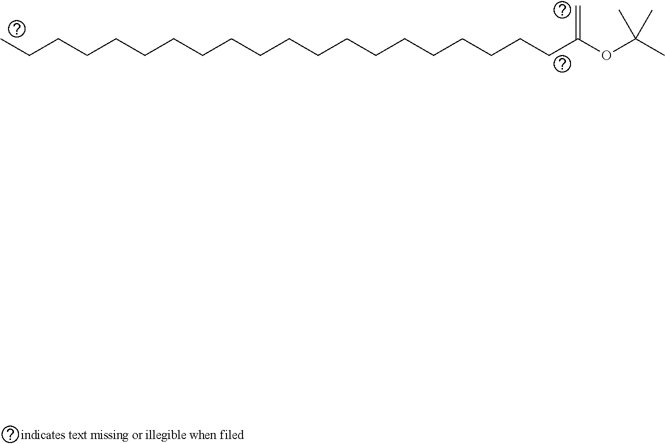 [SMILES: C=C(CCCCCCCCCCCCCCCCCCCC)OC(C)(C)C] | 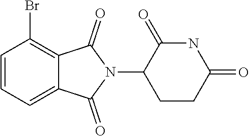 [SMILES: O=C1CCC(N2C(=O)c3cccc(Br)c3C2=O)C(=O)N1] |
| 44 | 1 | 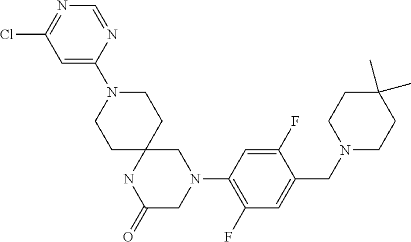 [SMILES: CC1(C)CCN(Cc2cc(F)c(N3CC(=O)NC4(CCN(c5cc(Cl)ncn5)CC4)C3)cc2F)CC1] | 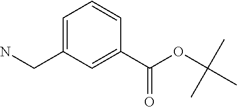 |
| | | 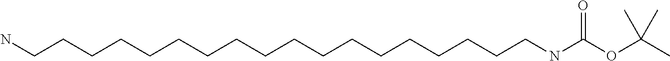 [SMILES: CC(C)(C)OC(=O)NCCCCCCCCCCCCCCCCCCN] | 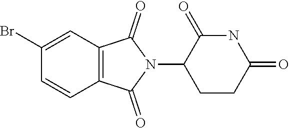 [SMILES: O=C1CCC(N2C(=O)c3ccc(Br)cc3C2=O)C(=O)N1] |
| 45 | 1 | 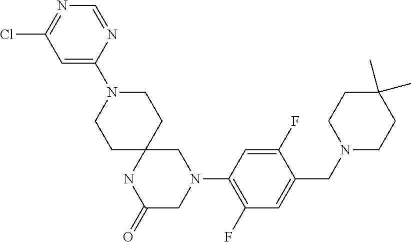 [SMILES: CC1(C)CCN(Cc2cc(F)c(N3CC(=O)NC4(CCN(c5cc(Cl)ncn5)CC4)C3)cc2F)CC1] | 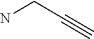 |
| | | 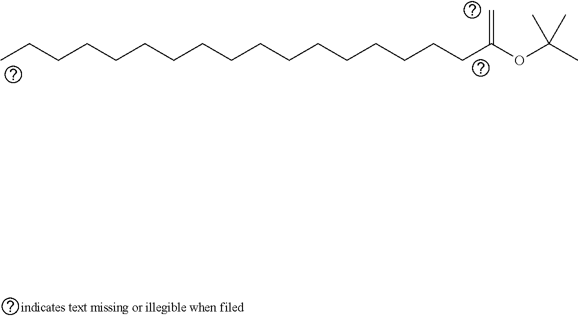 [SMILES: C=C(CCCCCCCCCCCCCCCCC)OC(C)(C)C] | 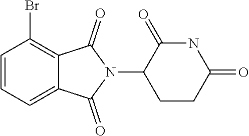 [SMILES: O=C1CCC(N2C(=O)c3cccc(Br)c3C2=O)C(=O)N1] |
| 46 | 1 | 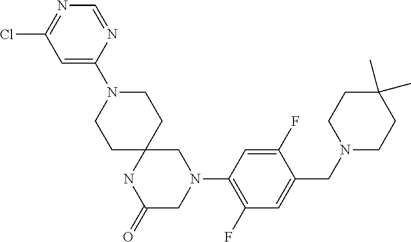 [SMILES: CC1(C)CCN(Cc2cc(F)c(N3CC(=O)NC4(CCN(c5cc(Cl)ncn5)CC4)C3)cc2F)CC1] | 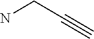 |
| | | 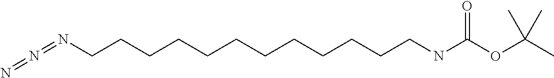 | 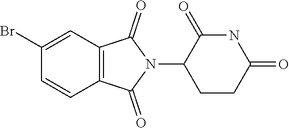 [SMILES: O=C1CCC(N2C(=O)c3ccc(Br)cc3C2=O)C(=O)N1] |
| 47 | 1 | 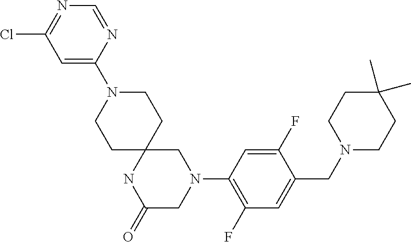 [SMILES: CC1(C)CCN(Cc2cc(F)c(N3CC(=O)NC4(CCN(c5cc(Cl)ncn5)CC4)C3)cc2F)CC1] | 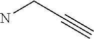 |
| | | 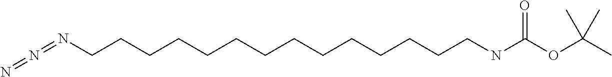 | 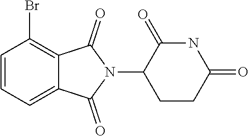 [SMILES: O=C1CCC(N2C(=O)c3cccc(Br)c3C2=O)C(=O)N1] |
| 48 | 1 | 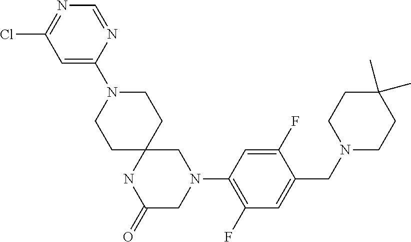 [SMILES: CC1(C)CCN(Cc2cc(F)c(N3CC(=O)NC4(CCN(c5cc(Cl)ncn5)CC4)C3)cc2F)CC1] | 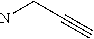 |
| | | 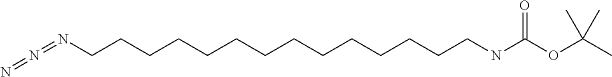 | 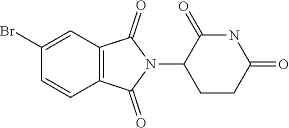 [SMILES: O=C1CCC(N2C(=O)c3ccc(Br)cc3C2=O)C(=O)N1] |
| 49 | 1 | 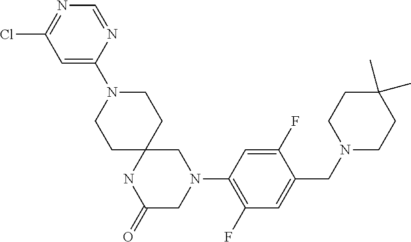 [SMILES: CC1(C)CCN(Cc2cc(F)c(N3CC(=O)NC4(CCN(c5cc(Cl)ncn5)CC4)C3)cc2F)CC1] | 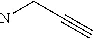 |
| | | 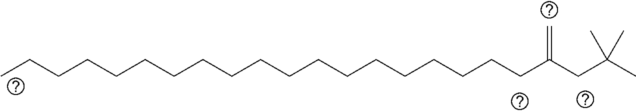 [SMILES: C=C(CCCCCCCCCCCCCCCCCCC)CC(C)(C)C] | 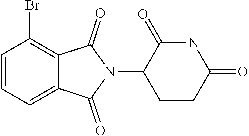 [SMILES: O=C1CCC(N2C(=O)c3cccc(Br)c3C2=O)C(=O)N1] |
| 50 | 1 | 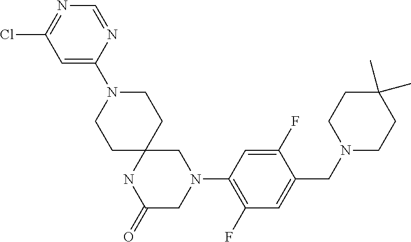 [SMILES: CC1(C)CCN(Cc2cc(F)c(N3CC(=O)NC4(CCN(c5cc(Cl)ncn5)CC4)C3)cc2F)CC1] | 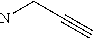 |
| | | 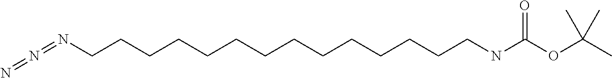 | 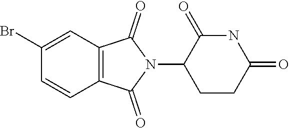 [SMILES: O=C1CCC(N2C(=O)c3ccc(Br)cc3C2=O)C(=O)N1] |
| 51 | 1 | 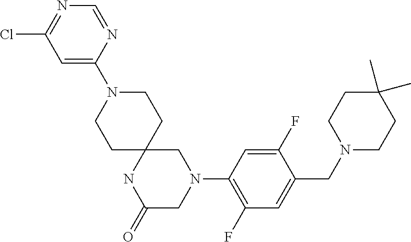 [SMILES: CC1(C)CCN(Cc2cc(F)c(N3CC(=O)NC4(CCN(c5cc(Cl)ncn5)CC4)C3)cc2F)CC1] | 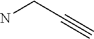 |
| | | 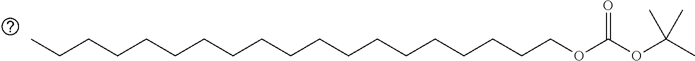 [SMILES: CCCCCCCCCCCCCCCCCCCOC(=O)OC(C)(C)C] | 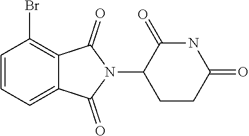 [SMILES: O=C1CCC(N2C(=O)c3cccc(Br)c3C2=O)C(=O)N1] |
| 52 | 1 | 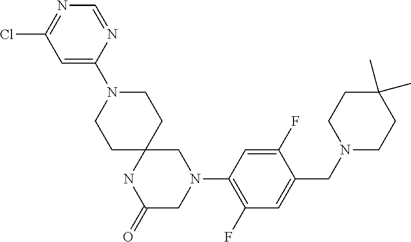 [SMILES: CC1(C)CCN(Cc2cc(F)c(N3CC(=O)NC4(CCN(c5cc(Cl)ncn5)CC4)C3)cc2F)CC1] | 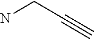 |
| | | 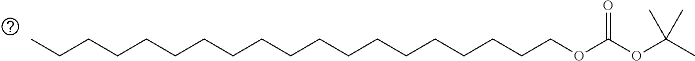 [SMILES: CCCCCCCCCCCCCCCCCCCOC(=O)OC(C)(C)C] | 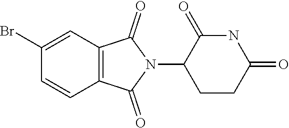 [SMILES: O=C1CCC(N2C(=O)c3ccc(Br)cc3C2=O)C(=O)N1] |
| 53 | 2 | 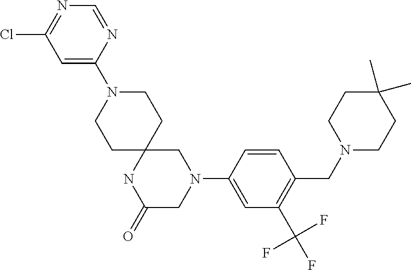 [SMILES: CC1(C)CCN(Cc2ccc(N3CC(=O)NC4(CCN(c5cc(Cl)ncn5)CC4)C3)cc2C(F)(F)F)CC1] | 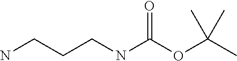 |
| | | 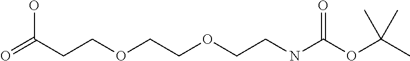 [SMILES: CC(C)(C)OC(=O)NCCOCCOCCC(=O)O] | 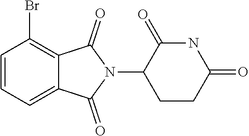 [SMILES: O=C1CCC(N2C(=O)c3cccc(Br)c3C2=O)C(=O)N1] |
| 54 | 2 | 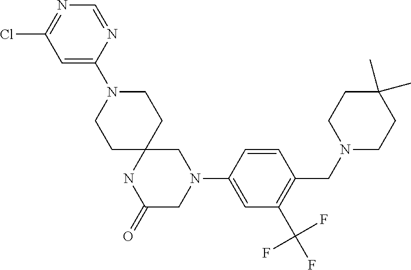 [SMILES: CC1(C)CCN(Cc2ccc(N3CC(=O)NC4(CCN(c5cc(Cl)ncn5)CC4)C3)cc2C(F)(F)F)CC1] | 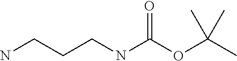 |
| | | 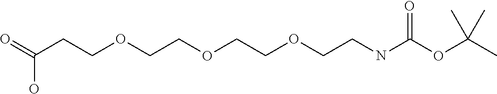 [SMILES: CC(C)(C)OC(=O)NCCOCCOCCOCCC(=O)O] | 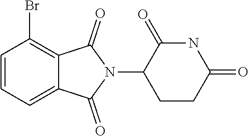 [SMILES: O=C1CCC(N2C(=O)c3cccc(Br)c3C2=O)C(=O)N1] |
| 55 | 2 | 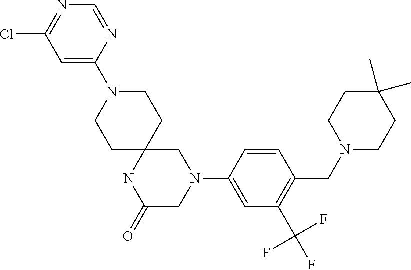 [SMILES: CC1(C)CCN(Cc2ccc(N3CC(=O)NC4(CCN(c5cc(Cl)ncn5)CC4)C3)cc2C(F)(F)F)CC1] | 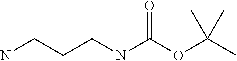 |
| | | 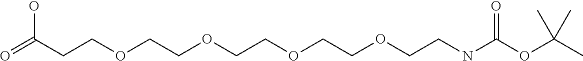 [SMILES: CC(C)(C)OC(=O)NCCOCCOCCOCCOCCC(=O)O] | 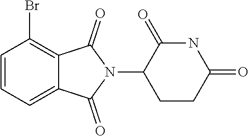 [SMILES: O=C1CCC(N2C(=O)c3cccc(Br)c3C2=O)C(=O)N1] |
| 56 | 2 | 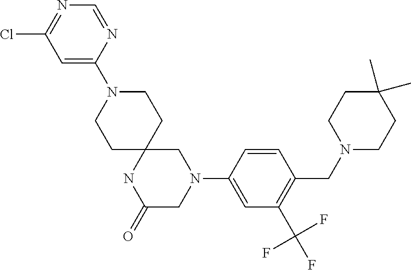 [SMILES: CC1(C)CCN(Cc2ccc(N3CC(=O)NC4(CCN(c5cc(Cl)ncn5)CC4)C3)cc2C(F)(F)F)CC1] | 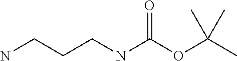 |
| | | 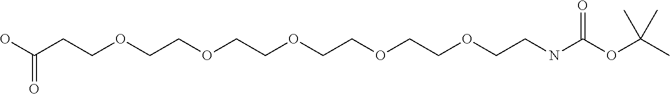 [SMILES: CC(C)(C)OC(=O)NCCOCCOCCOCCOCCOCCC(=O)O] | 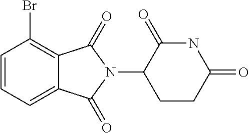 [SMILES: O=C1CCC(N2C(=O)c3cccc(Br)c3C2=O)C(=O)N1] |
| 57 | 2 | 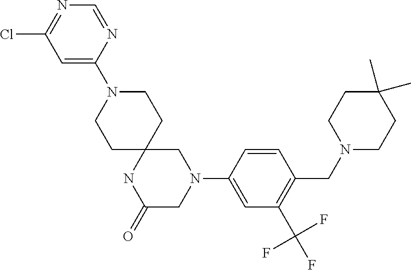 [SMILES: CC1(C)CCN(Cc2ccc(N3CC(=O)NC4(CCN(c5cc(Cl)ncn5)CC4)C3)cc2C(F)(F)F)CC1] | 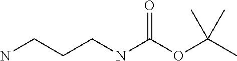 |
| | | 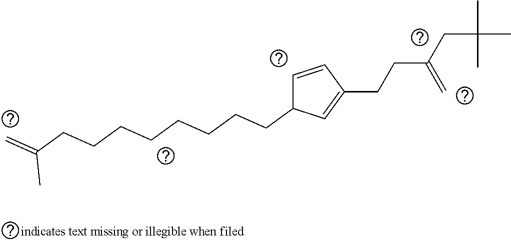 [SMILES: C=C(C)CCCCCCCCC1C=CC(CCC(=C)CC(C)(C)C)=C1] | 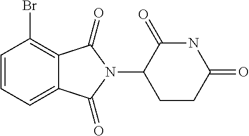 [SMILES: O=C1CCC(N2C(=O)c3cccc(Br)c3C2=O)C(=O)N1] |
| 58 | 2 | 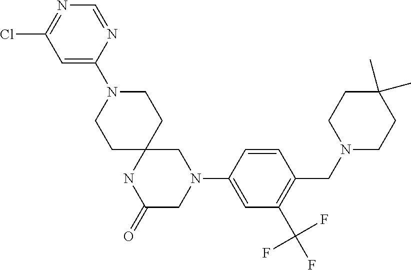 [SMILES: CC1(C)CCN(Cc2ccc(N3CC(=O)NC4(CCN(c5cc(Cl)ncn5)CC4)C3)cc2C(F)(F)F)CC1] | 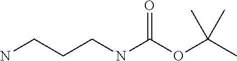 |
| | | 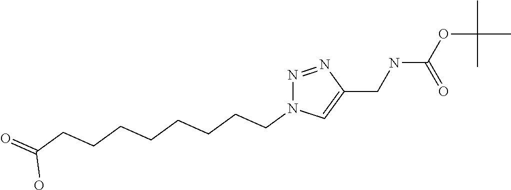 [SMILES: CC(C)(C)OC(=O)NCc1cn(CCCCCCCCC(=O)O)nn1] | 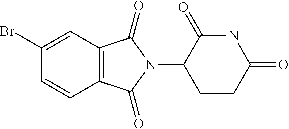 [SMILES: O=C1CCC(N2C(=O)c3ccc(Br)cc3C2=O)C(=O)N1] |
| 59 | 2 | 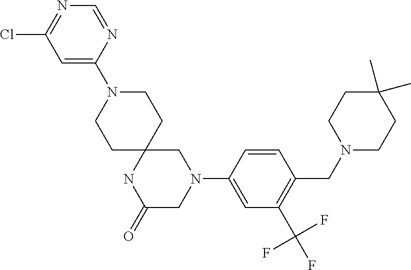 [SMILES: CC1(C)CCN(Cc2ccc(N3CC(=O)NC4(CCN(c5cc(Cl)ncn5)CC4)C3)cc2C(F)(F)F)CC1] | 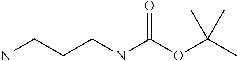 |
| | | 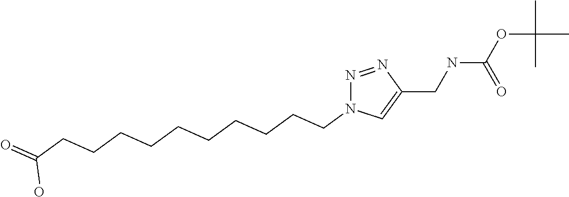 [SMILES: CC(C)(C)OC(=O)NCc1cn(CCCCCCCCCCC(=O)O)nn1] | 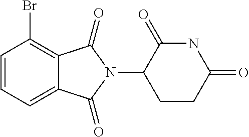 [SMILES: O=C1CCC(N2C(=O)c3cccc(Br)c3C2=O)C(=O)N1] |
| 60 | 2 | 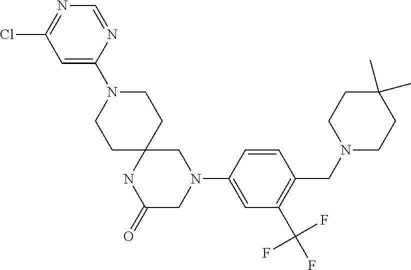 [SMILES: CC1(C)CCN(Cc2ccc(N3CC(=O)NC4(CCN(c5cc(Cl)ncn5)CC4)C3)cc2C(F)(F)F)CC1] | 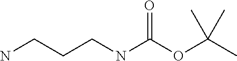 |
| | | 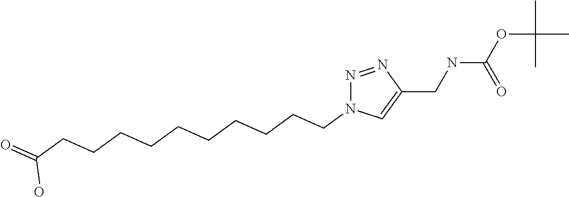 [SMILES: CC(C)(C)OC(=O)NCc1cn(CCCCCCCCCCC(=O)O)nn1] | 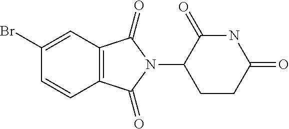 [SMILES: O=C1CCC(N2C(=O)c3ccc(Br)cc3C2=O)C(=O)N1] |
| 61 | 2 | 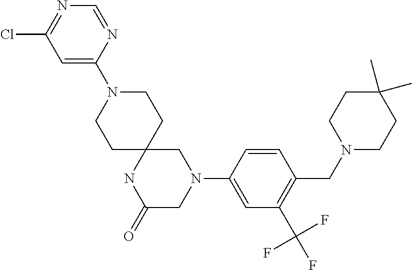 [SMILES: CC1(C)CCN(Cc2ccc(N3CC(=O)NC4(CCN(c5cc(Cl)ncn5)CC4)C3)cc2C(F)(F)F)CC1] | 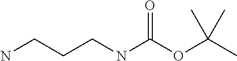 |
| | | 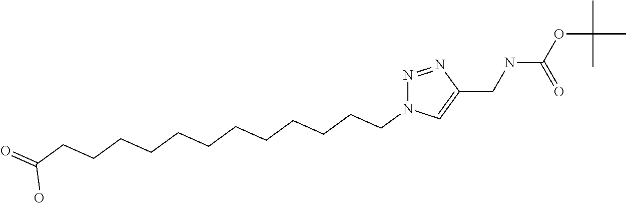 [SMILES: CC(C)(C)OC(=O)NCc1cn(CCCCCCCCCCCCC(=O)O)nn1] | 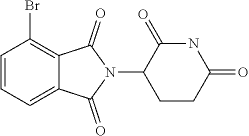 [SMILES: O=C1CCC(N2C(=O)c3cccc(Br)c3C2=O)C(=O)N1] |
| 62 | 2 | 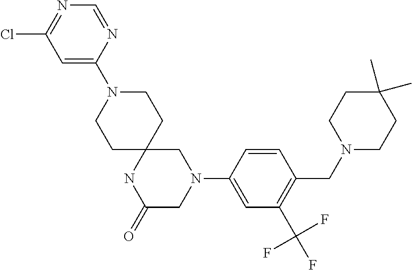 [SMILES: CC1(C)CCN(Cc2ccc(N3CC(=O)NC4(CCN(c5cc(Cl)ncn5)CC4)C3)cc2C(F)(F)F)CC1] | 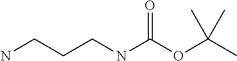 |
| | | 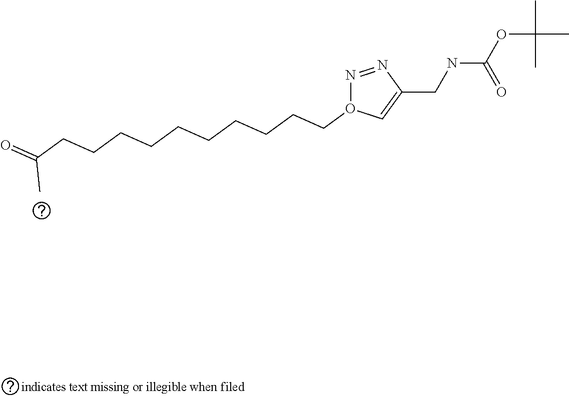 | 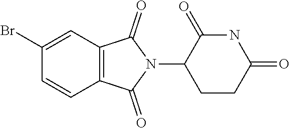 [SMILES: O=C1CCC(N2C(=O)c3ccc(Br)cc3C2=O)C(=O)N1] |
| 63 | 2 | 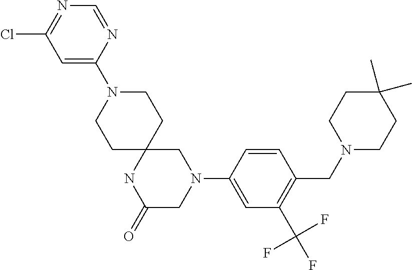 [SMILES: CC1(C)CCN(Cc2ccc(N3CC(=O)NC4(CCN(c5cc(Cl)ncn5)CC4)C3)cc2C(F)(F)F)CC1] | 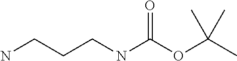 |
| | | 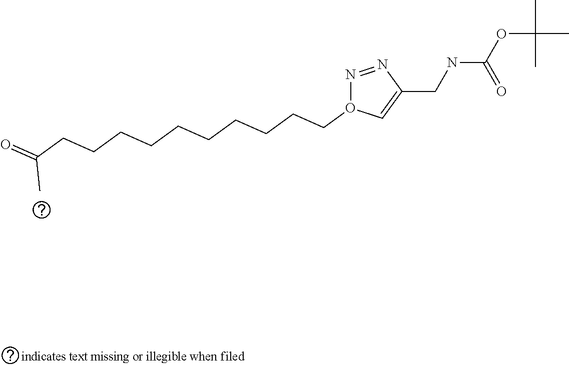 | 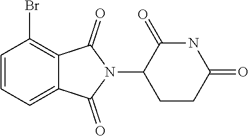 [SMILES: O=C1CCC(N2C(=O)c3cccc(Br)c3C2=O)C(=O)N1] |
| 64 | 2 | 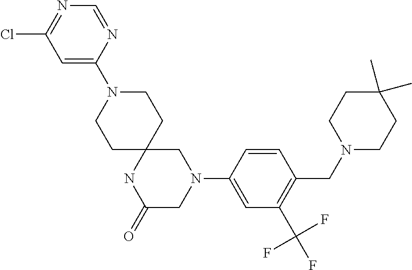 [SMILES: CC1(C)CCN(Cc2ccc(N3CC(=O)NC4(CCN(c5cc(Cl)ncn5)CC4)C3)cc2C(F)(F)F)CC1] | 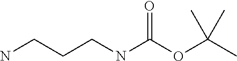 |
| | | 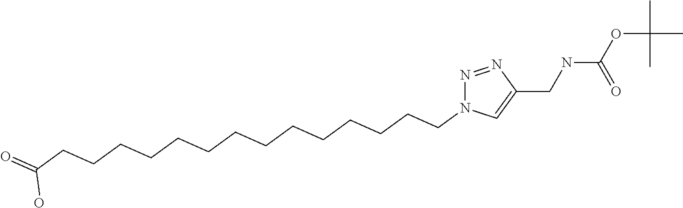 [SMILES: CC(C)(C)OC(=O)NCc1cn(CCCCCCCCCCCCCCC(=O)O)nn1] | 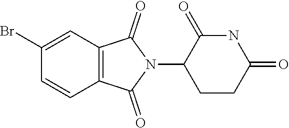 [SMILES: O=C1CCC(N2C(=O)c3ccc(Br)cc3C2=O)C(=O)N1] |
| 65 | 2 | 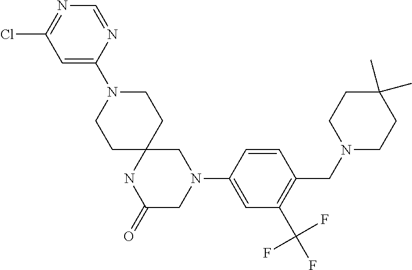 [SMILES: CC1(C)CCN(Cc2ccc(N3CC(=O)NC4(CCN(c5cc(Cl)ncn5)CC4)C3)cc2C(F)(F)F)CC1] | 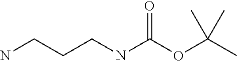 |
| | | 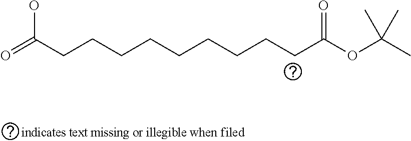 [SMILES: CC(C)(C)OC(=O)CCCCCCCCCC(=O)O] | 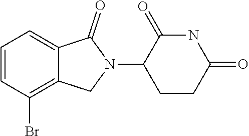 [SMILES: O=C1CCC(N2Cc3c(Br)cccc3C2=O)C(=O)N1] |
| 66 | 2 | 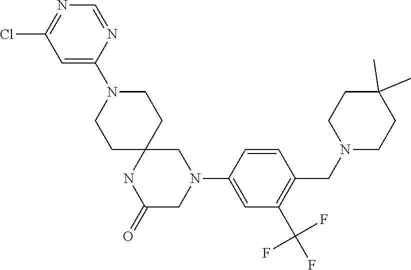 [SMILES: CC1(C)CCN(Cc2ccc(N3CC(=O)NC4(CCN(c5cc(Cl)ncn5)CC4)C3)cc2C(F)(F)F)CC1] | 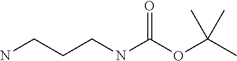 |
| | | 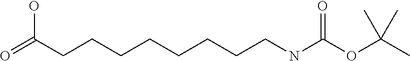 [SMILES: CC(C)(C)OC(=O)NCCCCCCCCC(=O)O] | 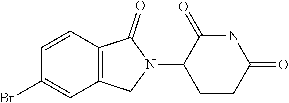 [SMILES: O=C1CCC(N2Cc3cc(Br)ccc3C2=O)C(=O)N1] |
| 67 | 2 | 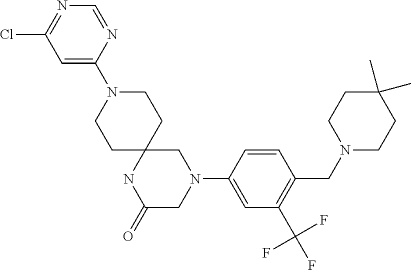 [SMILES: CC1(C)CCN(Cc2ccc(N3CC(=O)NC4(CCN(c5cc(Cl)ncn5)CC4)C3)cc2C(F)(F)F)CC1] | 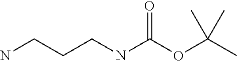 |
| | | 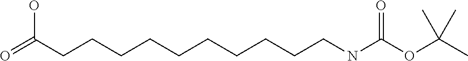 [SMILES: CC(C)(C)OC(=O)NCCCCCCCCCCC(=O)O] | 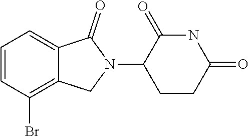 [SMILES: O=C1CCC(N2Cc3c(Br)cccc3C2=O)C(=O)N1] |
| 68 | 2 | 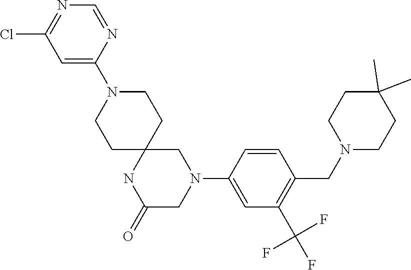 [SMILES: CC1(C)CCN(Cc2ccc(N3CC(=O)NC4(CCN(c5cc(Cl)ncn5)CC4)C3)cc2C(F)(F)F)CC1] | 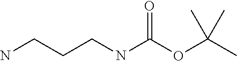 |
| | | 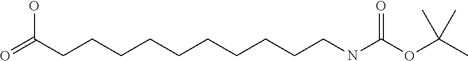 [SMILES: CC(C)(C)OC(=O)NCCCCCCCCCCC(=O)O] | 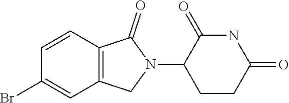 [SMILES: O=C1CCC(N2Cc3cc(Br)ccc3C2=O)C(=O)N1] |
| 69 | 2 | 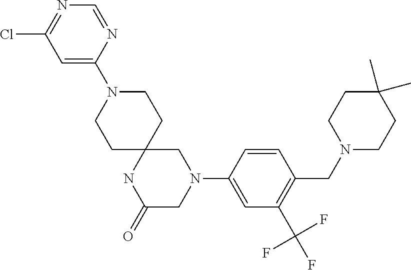 [SMILES: CC1(C)CCN(Cc2ccc(N3CC(=O)NC4(CCN(c5cc(Cl)ncn5)CC4)C3)cc2C(F)(F)F)CC1] | 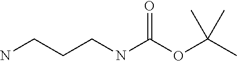 |
| | | 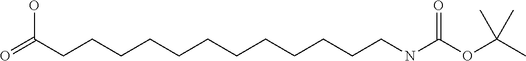 [SMILES: CC(C)(C)OC(=O)NCCCCCCCCCCCCC(=O)O] | 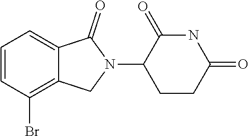 [SMILES: O=C1CCC(N2Cc3c(Br)cccc3C2=O)C(=O)N1] |
| 70 | 2 | 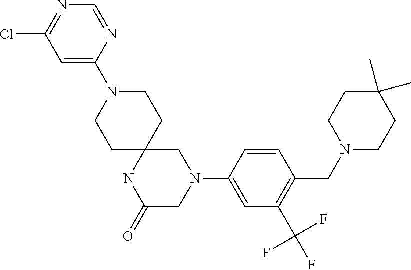 [SMILES: CC1(C)CCN(Cc2ccc(N3CC(=O)NC4(CCN(c5cc(Cl)ncn5)CC4)C3)cc2C(F)(F)F)CC1] | 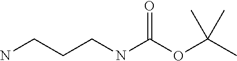 |
| | | 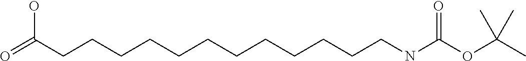 [SMILES: CC(C)(C)OC(=O)NCCCCCCCCCCCCC(=O)O] | 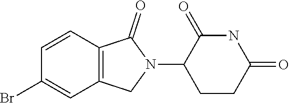 [SMILES: O=C1CCC(N2Cc3cc(Br)ccc3C2=O)C(=O)N1] |
| 71 | 2 | 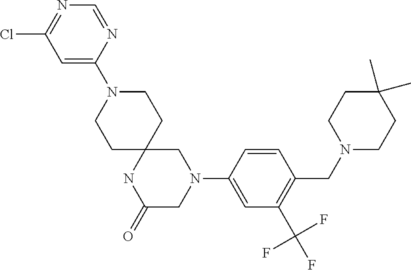 [SMILES: CC1(C)CCN(Cc2ccc(N3CC(=O)NC4(CCN(c5cc(Cl)ncn5)CC4)C3)cc2C(F)(F)F)CC1] | 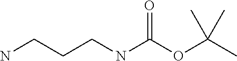 |
| | | 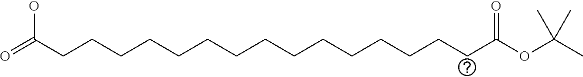 [SMILES: CC(C)(C)OC(=O)CCCCCCCCCCCCCCCC(=O)O] | 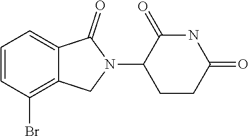 [SMILES: O=C1CCC(N2Cc3c(Br)cccc3C2=O)C(=O)N1] |
| 72 | 2 | 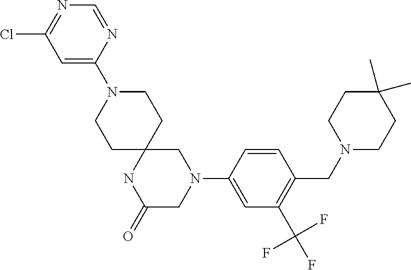 [SMILES: CC1(C)CCN(Cc2ccc(N3CC(=O)NC4(CCN(c5cc(Cl)ncn5)CC4)C3)cc2C(F)(F)F)CC1] | 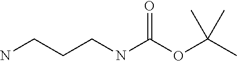 |
| | | 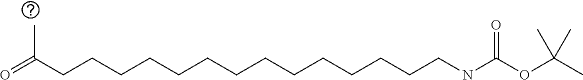 [SMILES: CC(=O)CCCCCCCCCCCCCCNC(=O)OC(C)(C)C] | 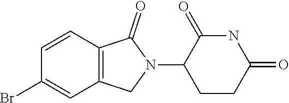 [SMILES: O=C1CCC(N2Cc3cc(Br)ccc3C2=O)C(=O)N1] |
| 73 | 2 | 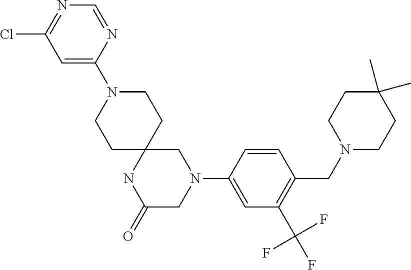 [SMILES: CC1(C)CCN(Cc2ccc(N3CC(=O)NC4(CCN(c5cc(Cl)ncn5)CC4)C3)cc2C(F)(F)F)CC1] | 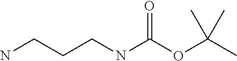 |
| | | 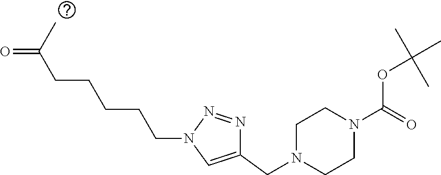 [SMILES: CC(=O)CCCCCn1cc(CN2CCN(C(=O)OC(C)(C)C)CC2)nn1] | 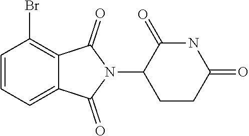 [SMILES: O=C1CCC(N2C(=O)c3cccc(Br)c3C2=O)C(=O)N1] |
| 74 | 2 | 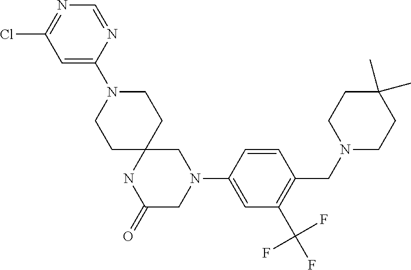 [SMILES: CC1(C)CCN(Cc2ccc(N3CC(=O)NC4(CCN(c5cc(Cl)ncn5)CC4)C3)cc2C(F)(F)F)CC1] | 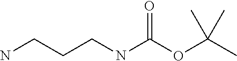 |
| | | 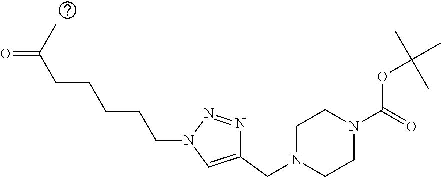 [SMILES: CC(=O)CCCCCn1cc(CN2CCN(C(=O)OC(C)(C)C)CC2)nn1] | 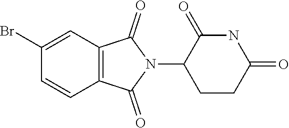 [SMILES: O=C1CCC(N2C(=O)c3ccc(Br)cc3C2=O)C(=O)N1] |
| 75 | 2 | 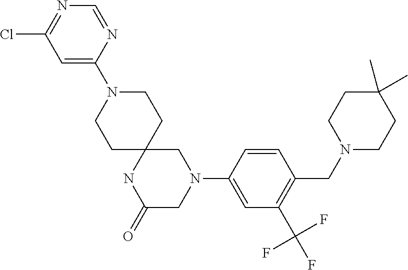 [SMILES: CC1(C)CCN(Cc2ccc(N3CC(=O)NC4(CCN(c5cc(Cl)ncn5)CC4)C3)cc2C(F)(F)F)CC1] | 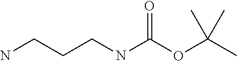 |
| | | 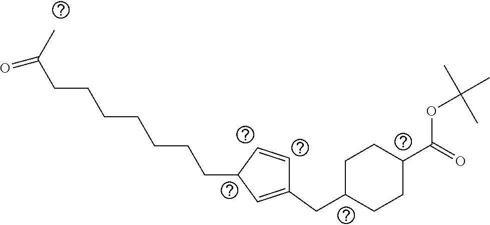 [SMILES: CC(=O)CCCCCCCC1C=CC(CC2CCC(C(=O)OC(C)(C)C)CC2)=C1] | 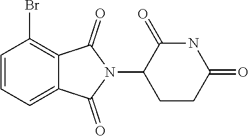 [SMILES: O=C1CCC(N2C(=O)c3cccc(Br)c3C2=O)C(=O)N1] |
| 76 | 2 | 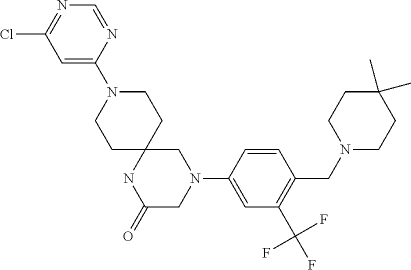 [SMILES: CC1(C)CCN(Cc2ccc(N3CC(=O)NC4(CCN(c5cc(Cl)ncn5)CC4)C3)cc2C(F)(F)F)CC1] | 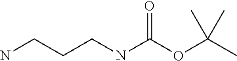 |
| | | 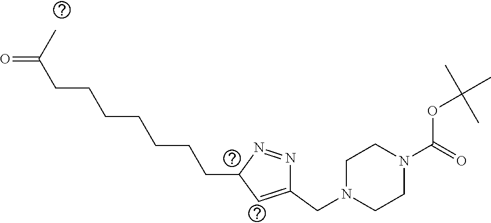 [SMILES: CC(=O)CCCCCCCC1C=C(CN2CCN(C(=O)OC(C)(C)C)CC2)N=N1] | 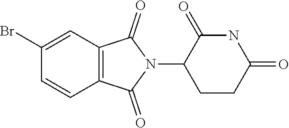 [SMILES: O=C1CCC(N2C(=O)c3ccc(Br)cc3C2=O)C(=O)N1] |
| 77 | 2 | 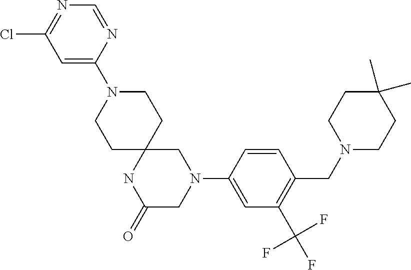 [SMILES: CC1(C)CCN(Cc2ccc(N3CC(=O)NC4(CCN(c5cc(Cl)ncn5)CC4)C3)cc2C(F)(F)F)CC1] | 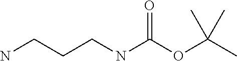 |
| | | 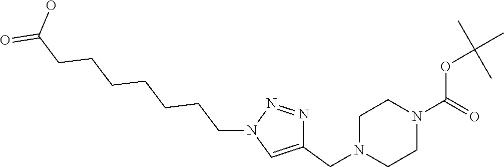 [SMILES: CC(C)(C)OC(=O)N1CCN(Cc2cn(CCCCCCCC(=O)O)nn2)CC1] | 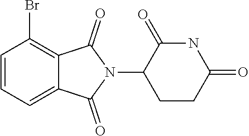 [SMILES: O=C1CCC(N2C(=O)c3cccc(Br)c3C2=O)C(=O)N1] |
| 8 | 2 | 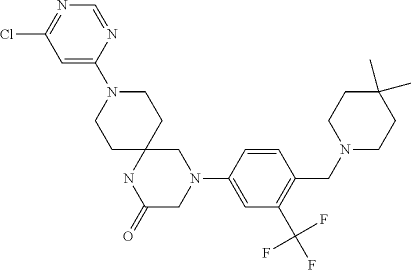 [SMILES: CC1(C)CCN(Cc2ccc(N3CC(=O)NC4(CCN(c5cc(Cl)ncn5)CC4)C3)cc2C(F)(F)F)CC1] | 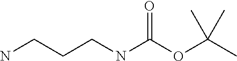 |
| | | 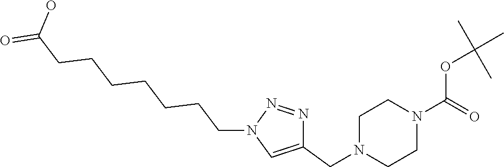 [SMILES: CC(C)(C)OC(=O)N1CCN(Cc2cn(CCCCCCCC(=O)O)nn2)CC1] | 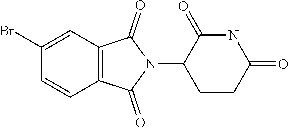 [SMILES: O=C1CCC(N2C(=O)c3ccc(Br)cc3C2=O)C(=O)N1] |
| 79 | 2 | 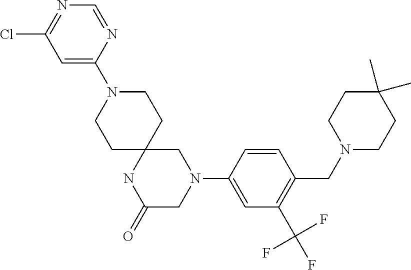 [SMILES: CC1(C)CCN(Cc2ccc(N3CC(=O)NC4(CCN(c5cc(Cl)ncn5)CC4)C3)cc2C(F)(F)F)CC1] | 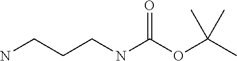 |
| | | 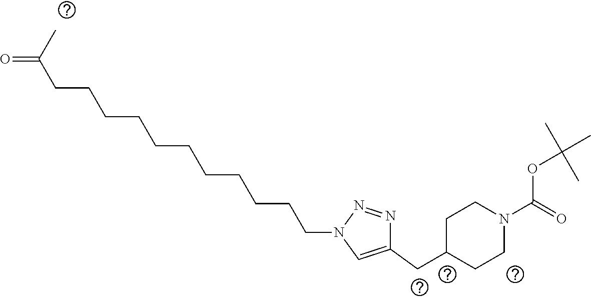 [SMILES: CC(=O)CCCCCCCCCCCn1cc(CC2CCN(C(=O)OC(C)(C)C)CC2)nn1] | 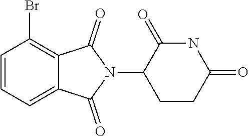 [SMILES: O=C1CCC(N2C(=O)c3cccc(Br)c3C2=O)C(=O)N1] |
| 80 | 2 | 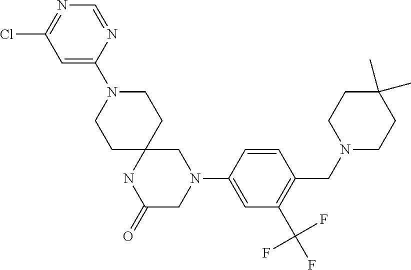 [SMILES: CC1(C)CCN(Cc2ccc(N3CC(=O)NC4(CCN(c5cc(Cl)ncn5)CC4)C3)cc2C(F)(F)F)CC1] | 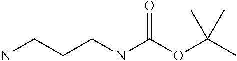 |
| | | 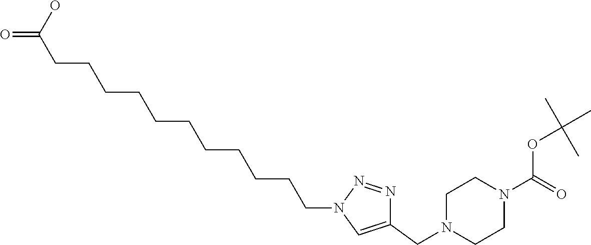 [SMILES: CC(C)(C)OC(=O)N1CCN(Cc2cn(CCCCCCCCCCCC(=O)O)nn2)CC1] | 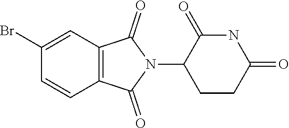 [SMILES: O=C1CCC(N2C(=O)c3ccc(Br)cc3C2=O)C(=O)N1] |
| 81 | 2 | 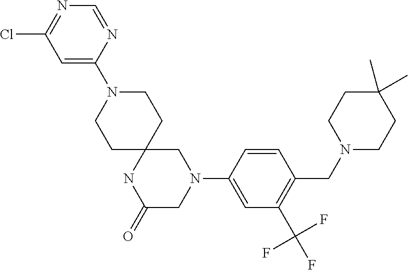 [SMILES: CC1(C)CCN(Cc2ccc(N3CC(=O)NC4(CCN(c5cc(Cl)ncn5)CC4)C3)cc2C(F)(F)F)CC1] | 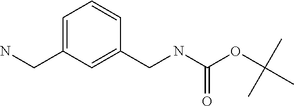 [SMILES: CC(C)(C)OC(=O)NCc1cccc(CN)c1] |
| | | 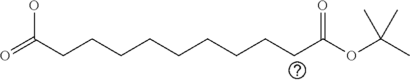 [SMILES: CC(C)(C)OC(=O)CCCCCCCCCC(=O)O] | 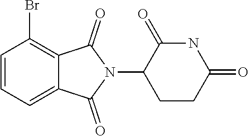 [SMILES: O=C1CCC(N2C(=O)c3cccc(Br)c3C2=O)C(=O)N1] |
| 82 | 2 | 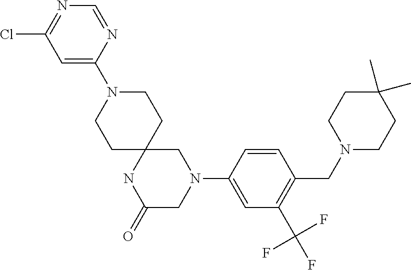 [SMILES: CC1(C)CCN(Cc2ccc(N3CC(=O)NC4(CCN(c5cc(Cl)ncn5)CC4)C3)cc2C(F)(F)F)CC1] | 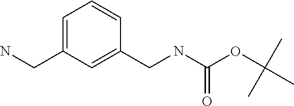 [SMILES: CC(C)(C)OC(=O)NCc1cccc(CN)c1] |
| | | 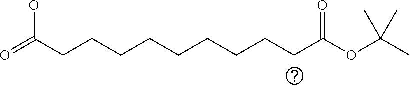 [SMILES: CC(C)(C)OC(=O)CCCCCCCCCC(=O)O] | 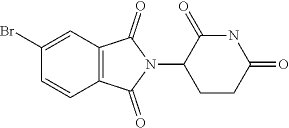 [SMILES: O=C1CCC(N2C(=O)c3ccc(Br)cc3C2=O)C(=O)N1] |
| 83 | 2 | 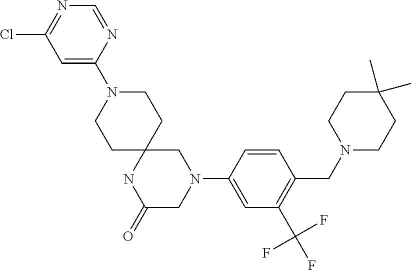 [SMILES: CC1(C)CCN(Cc2ccc(N3CC(=O)NC4(CCN(c5cc(Cl)ncn5)CC4)C3)cc2C(F)(F)F)CC1] | 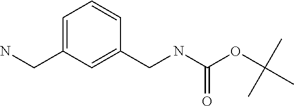 [SMILES: CC(C)(C)OC(=O)NCc1cccc(CN)c1] |
| | | 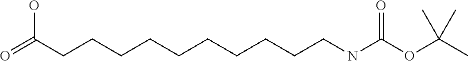 [SMILES: CC(C)(C)OC(=O)NCCCCCCCCCCC(=O)O] | 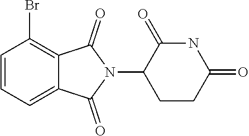 [SMILES: O=C1CCC(N2C(=O)c3cccc(Br)c3C2=O)C(=O)N1] |
| 84 | 2 | 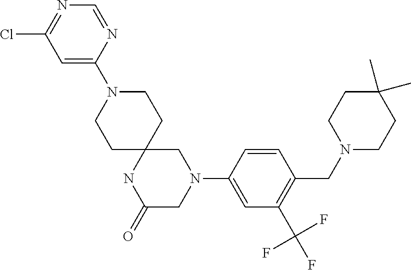 [SMILES: CC1(C)CCN(Cc2ccc(N3CC(=O)NC4(CCN(c5cc(Cl)ncn5)CC4)C3)cc2C(F)(F)F)CC1] | 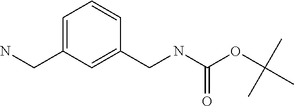 [SMILES: CC(C)(C)OC(=O)NCc1cccc(CN)c1] |
| | | 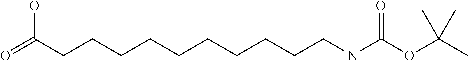 [SMILES: CC(C)(C)OC(=O)NCCCCCCCCCCC(=O)O] | 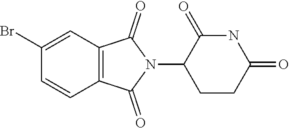 [SMILES: O=C1CCC(N2C(=O)c3ccc(Br)cc3C2=O)C(=O)N1] |
| 85 | 2 | 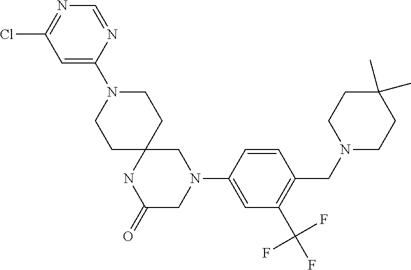 [SMILES: CC1(C)CCN(Cc2ccc(N3CC(=O)NC4(CCN(c5cc(Cl)ncn5)CC4)C3)cc2C(F)(F)F)CC1] | 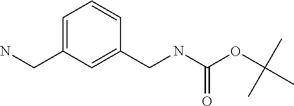 [SMILES: CC(C)(C)OC(=O)NCc1cccc(CN)c1] |
| | | 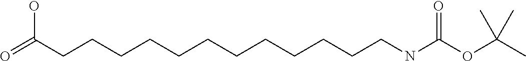 [SMILES: CC(C)(C)OC(=O)NCCCCCCCCCCCCC(=O)O] | 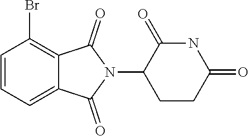 [SMILES: O=C1CCC(N2C(=O)c3cccc(Br)c3C2=O)C(=O)N1] |
| 86 | 2 | 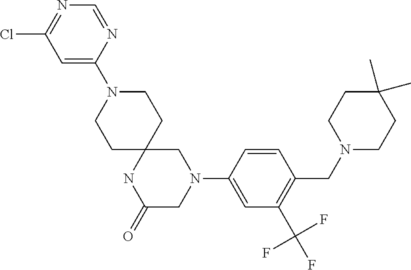 [SMILES: CC1(C)CCN(Cc2ccc(N3CC(=O)NC4(CCN(c5cc(Cl)ncn5)CC4)C3)cc2C(F)(F)F)CC1] | 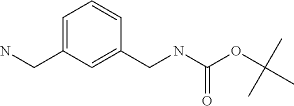 [SMILES: CC(C)(C)OC(=O)NCc1cccc(CN)c1] |
| | | 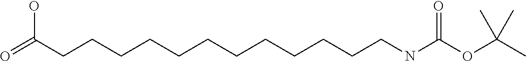 [SMILES: CC(C)(C)OC(=O)NCCCCCCCCCCCCC(=O)O] | 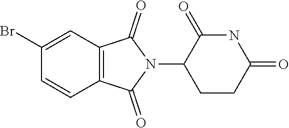 [SMILES: O=C1CCC(N2C(=O)c3ccc(Br)cc3C2=O)C(=O)N1] |
| 87 | 2 | 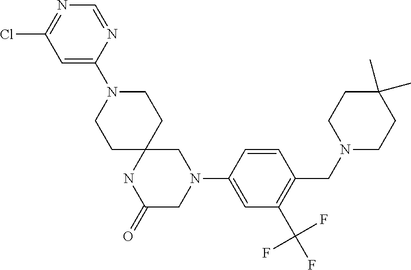 [SMILES: CC1(C)CCN(Cc2ccc(N3CC(=O)NC4(CCN(c5cc(Cl)ncn5)CC4)C3)cc2C(F)(F)F)CC1] | 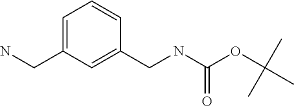 [SMILES: CC(C)(C)OC(=O)NCc1cccc(CN)c1] |
| | | 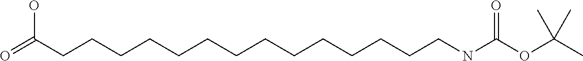 [SMILES: CC(C)(C)OC(=O)NCCCCCCCCCCCCCCC(=O)O] | 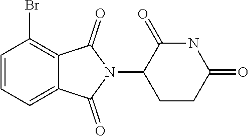 [SMILES: O=C1CCC(N2C(=O)c3cccc(Br)c3C2=O)C(=O)N1] |
| 88 | 2 | 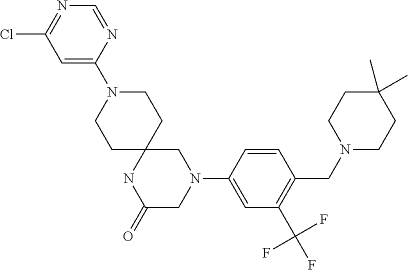 [SMILES: CC1(C)CCN(Cc2ccc(N3CC(=O)NC4(CCN(c5cc(Cl)ncn5)CC4)C3)cc2C(F)(F)F)CC1] | 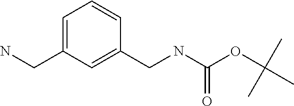 [SMILES: CC(C)(C)OC(=O)NCc1cccc(CN)c1] |
| | | 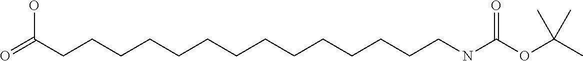 [SMILES: CC(C)(C)OC(=O)NCCCCCCCCCCCCCCC(=O)O] | 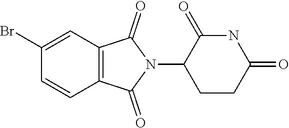 [SMILES: O=C1CCC(N2C(=O)c3ccc(Br)cc3C2=O)C(=O)N1] |
| 89 | 2 | 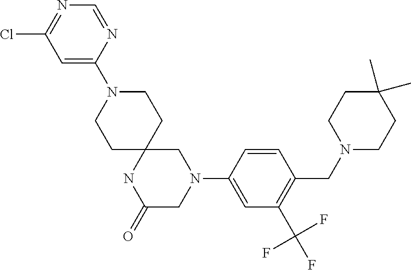 [SMILES: CC1(C)CCN(Cc2ccc(N3CC(=O)NC4(CCN(c5cc(Cl)ncn5)CC4)C3)cc2C(F)(F)F)CC1] | 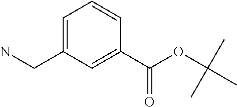 |
| | | 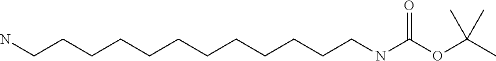 [SMILES: CC(C)(C)OC(=O)NCCCCCCCCCCCCN] | 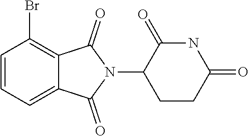 [SMILES: O=C1CCC(N2C(=O)c3cccc(Br)c3C2=O)C(=O)N1] |
| 90 | 2 | 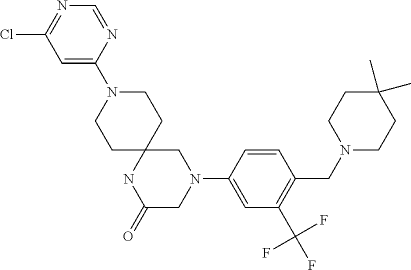 [SMILES: CC1(C)CCN(Cc2ccc(N3CC(=O)NC4(CCN(c5cc(Cl)ncn5)CC4)C3)cc2C(F)(F)F)CC1] | 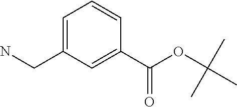 |
| | | 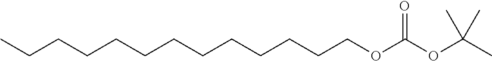 [SMILES: CCCCCCCCCCCCCOC(=O)OC(C)(C)C] | 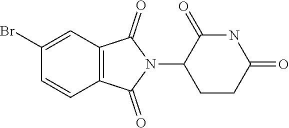 [SMILES: O=C1CCC(N2C(=O)c3ccc(Br)cc3C2=O)C(=O)N1] |
| 91 | 2 | 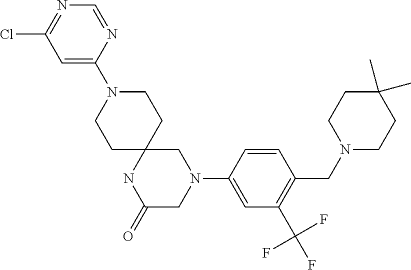 [SMILES: CC1(C)CCN(Cc2ccc(N3CC(=O)NC4(CCN(c5cc(Cl)ncn5)CC4)C3)cc2C(F)(F)F)CC1] | 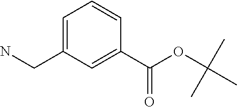 |
| | | 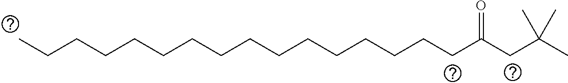 [SMILES: CCCCCCCCCCCCCCCCC(=O)CC(C)(C)C] | 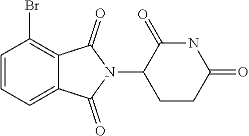 [SMILES: O=C1CCC(N2C(=O)c3cccc(Br)c3C2=O)C(=O)N1] |
| 92 | 2 | 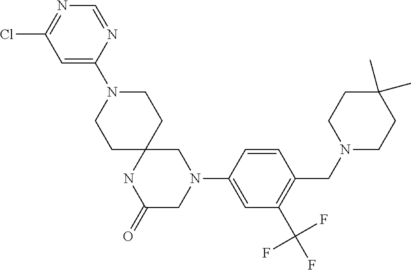 [SMILES: CC1(C)CCN(Cc2ccc(N3CC(=O)NC4(CCN(c5cc(Cl)ncn5)CC4)C3)cc2C(F)(F)F)CC1] | 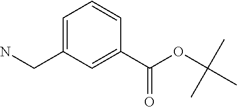 |
| | | 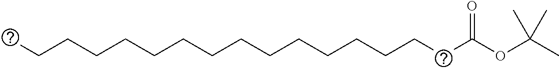 [SMILES: CCCCCCCCCCCCCCCCC(=O)OC(C)(C)C] | 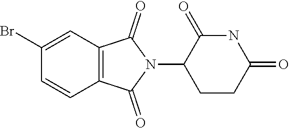 [SMILES: O=C1CCC(N2C(=O)c3ccc(Br)cc3C2=O)C(=O)N1] |
| 93 | 2 | 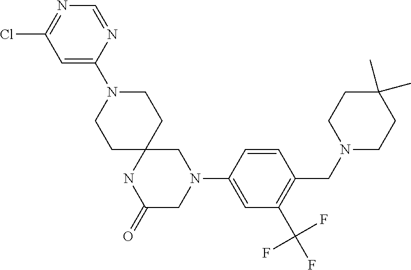 [SMILES: CC1(C)CCN(Cc2ccc(N3CC(=O)NC4(CCN(c5cc(Cl)ncn5)CC4)C3)cc2C(F)(F)F)CC1] | 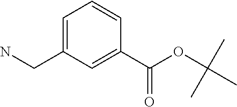 |
| | | 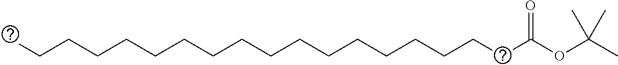 [SMILES: CCCCCCCCCCCCCCCCCCC(=O)OC(C)(C)C] | 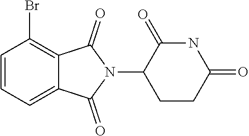 [SMILES: O=C1CCC(N2C(=O)c3cccc(Br)c3C2=O)C(=O)N1] |
| 94 | 2 | 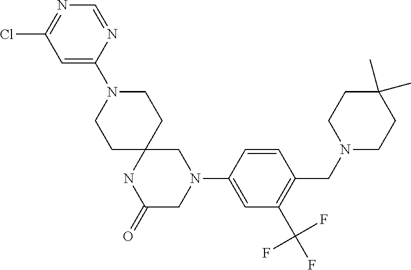 [SMILES: CC1(C)CCN(Cc2ccc(N3CC(=O)NC4(CCN(c5cc(Cl)ncn5)CC4)C3)cc2C(F)(F)F)CC1] | 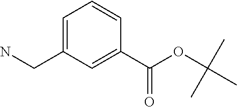 |
| | | 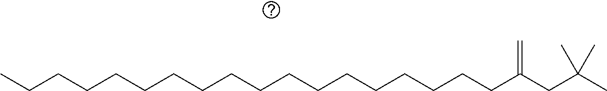 [SMILES: C=C(CCCCCCCCCCCCCCCCCC)CC(C)(C)C] | 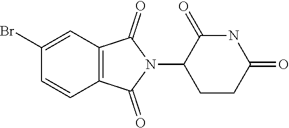 [SMILES: O=C1CCC(N2C(=O)c3ccc(Br)cc3C2=O)C(=O)N1] |
| 95 | 2 | 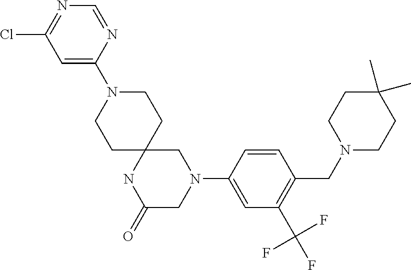 [SMILES: CC1(C)CCN(Cc2ccc(N3CC(=O)NC4(CCN(c5cc(Cl)ncn5)CC4)C3)cc2C(F)(F)F)CC1] | 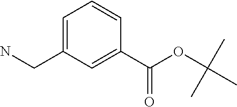 |
| | | 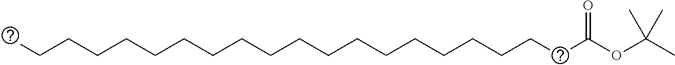 [SMILES: CCCCCCCCCCCCCCCCCCCCC(=O)OC(C)(C)C] | 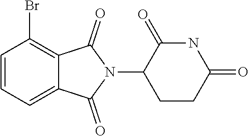 [SMILES: O=C1CCC(N2C(=O)c3cccc(Br)c3C2=O)C(=O)N1] |
| 96 | 2 | 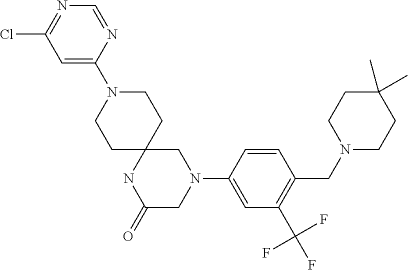 [SMILES: CC1(C)CCN(Cc2ccc(N3CC(=O)NC4(CCN(c5cc(Cl)ncn5)CC4)C3)cc2C(F)(F)F)CC1] | 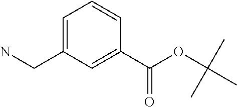 |
| | | 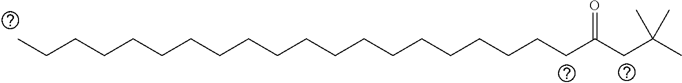 [SMILES: CCCCCCCCCCCCCCCCCCCCC(=O)CC(C)(C)C] | 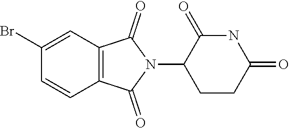 [SMILES: O=C1CCC(N2C(=O)c3ccc(Br)cc3C2=O)C(=O)N1] |
| 97 | 2 | 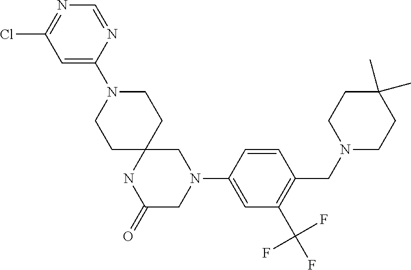 [SMILES: CC1(C)CCN(Cc2ccc(N3CC(=O)NC4(CCN(c5cc(Cl)ncn5)CC4)C3)cc2C(F)(F)F)CC1] | 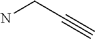 |
| | | 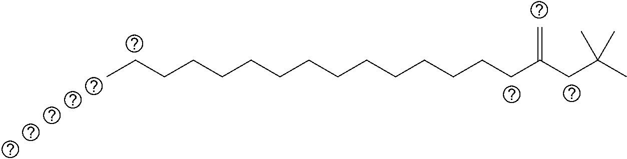 [SMILES: C=C(CCCCCCCCCCCCCCC)CC(C)(C)C] | 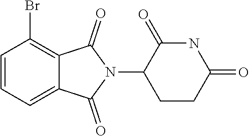 [SMILES: O=C1CCC(N2C(=O)c3cccc(Br)c3C2=O)C(=O)N1] |
| 98 | 2 | 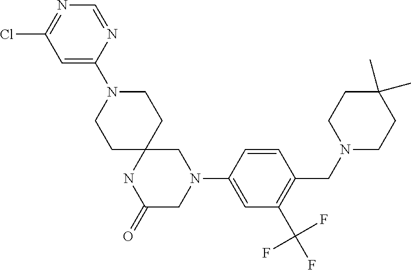 [SMILES: CC1(C)CCN(Cc2ccc(N3CC(=O)NC4(CCN(c5cc(Cl)ncn5)CC4)C3)cc2C(F)(F)F)CC1] | 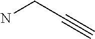 |
| | | 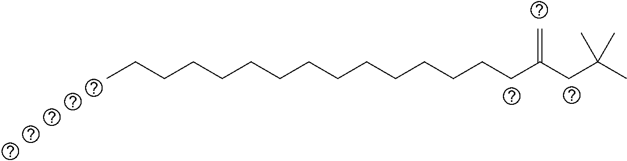 [SMILES: C=C(CCCCCCCCCCCCCCC)CC(C)(C)C] | 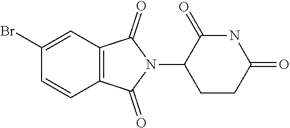 [SMILES: O=C1CCC(N2C(=O)c3ccc(Br)cc3C2=O)C(=O)N1] |
| 99 | 2 | 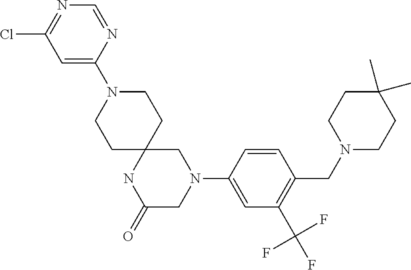 [SMILES: CC1(C)CCN(Cc2ccc(N3CC(=O)NC4(CCN(c5cc(Cl)ncn5)CC4)C3)cc2C(F)(F)F)CC1] | 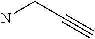 |
| | | 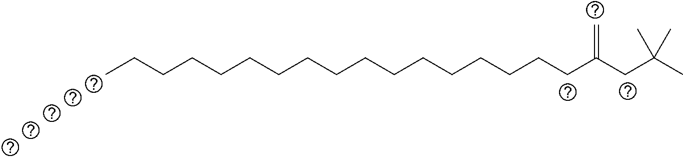 [SMILES: C=C(CCCCCCCCCCCCCCCCC)CC(C)(C)C] | 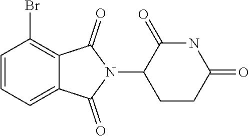 [SMILES: O=C1CCC(N2C(=O)c3cccc(Br)c3C2=O)C(=O)N1] |
| 100 | 2 | 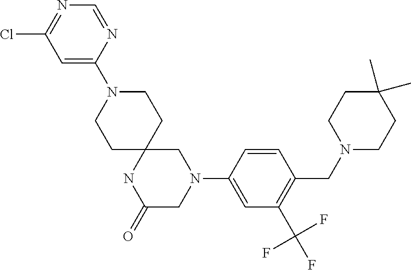 [SMILES: CC1(C)CCN(Cc2ccc(N3CC(=O)NC4(CCN(c5cc(Cl)ncn5)CC4)C3)cc2C(F)(F)F)CC1] | 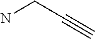 |
| | | 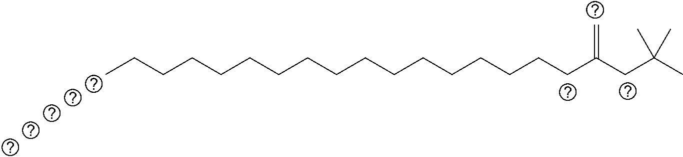 [SMILES: C=C(CCCCCCCCCCCCCCCCC)CC(C)(C)C] | 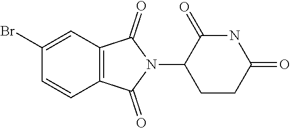 [SMILES: O=C1CCC(N2C(=O)c3ccc(Br)cc3C2=O)C(=O)N1] |
| 101 | 2 | 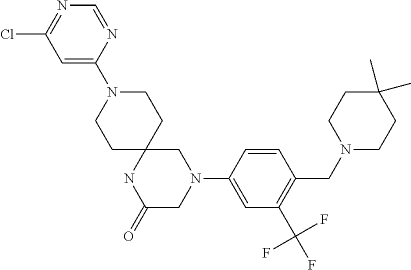 [SMILES: CC1(C)CCN(Cc2ccc(N3CC(=O)NC4(CCN(c5cc(Cl)ncn5)CC4)C3)cc2C(F)(F)F)CC1] | 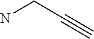 |
| | | 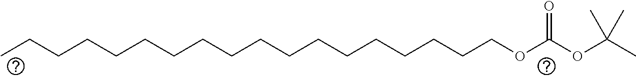 [SMILES: CCCCCCCCCCCCCCCCCCOC(=O)OC(C)(C)C] | 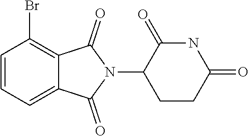 [SMILES: O=C1CCC(N2C(=O)c3cccc(Br)c3C2=O)C(=O)N1] |
| 101 | 2 | 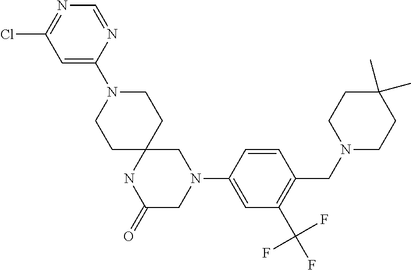 [SMILES: CC1(C)CCN(Cc2ccc(N3CC(=O)NC4(CCN(c5cc(Cl)ncn5)CC4)C3)cc2C(F)(F)F)CC1] | 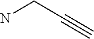 |
| | | 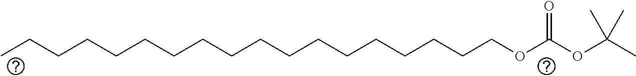 [SMILES: CCCCCCCCCCCCCCCCCCOC(=O)OC(C)(C)C] | 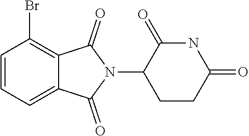 [SMILES: O=C1CCC(N2C(=O)c3cccc(Br)c3C2=O)C(=O)N1] |
| 102 | 2 | 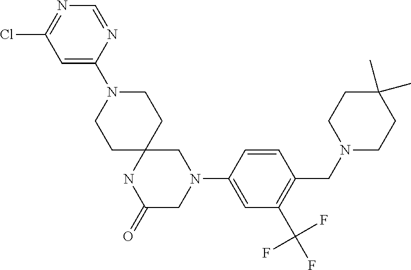 [SMILES: CC1(C)CCN(Cc2ccc(N3CC(=O)NC4(CCN(c5cc(Cl)ncn5)CC4)C3)cc2C(F)(F)F)CC1] | 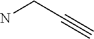 |
| | | 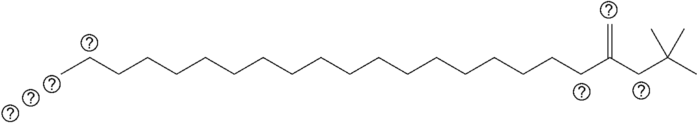 [SMILES: C=C(CCCCCCCCCCCCCCCCCCC)CC(C)(C)C] | 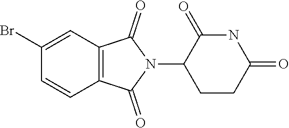 [SMILES: O=C1CCC(N2C(=O)c3ccc(Br)cc3C2=O)C(=O)N1] |
| 103 | 2 | 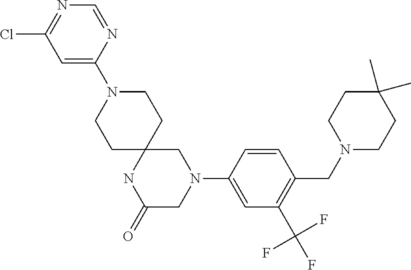 [SMILES: CC1(C)CCN(Cc2ccc(N3CC(=O)NC4(CCN(c5cc(Cl)ncn5)CC4)C3)cc2C(F)(F)F)CC1] | 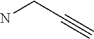 |
| | | 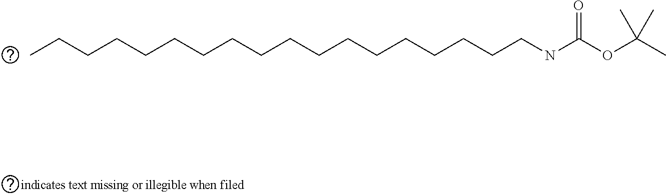 [SMILES: CCCCCCCCCCCCCCCCCCNC(=O)OC(C)(C)C] | 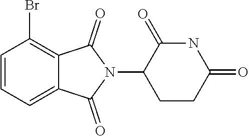 [SMILES: O=C1CCC(N2C(=O)c3cccc(Br)c3C2=O)C(=O)N1] |
| 104 | 2 | 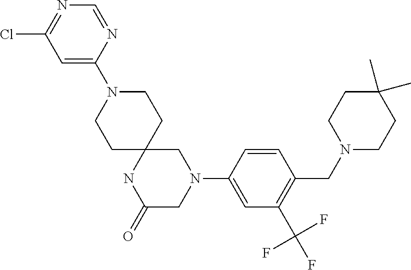 [SMILES: CC1(C)CCN(Cc2ccc(N3CC(=O)NC4(CCN(c5cc(Cl)ncn5)CC4)C3)cc2C(F)(F)F)CC1] | 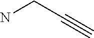 |
| | | 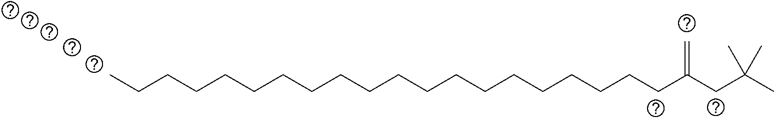 [SMILES: C=C(CCCCCCCCCCCCCCCCCCCC)CC(C)(C)C] | 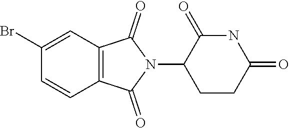 [SMILES: O=C1CCC(N2C(=O)c3ccc(Br)cc3C2=O)C(=O)N1] |
| 105 | 3 | 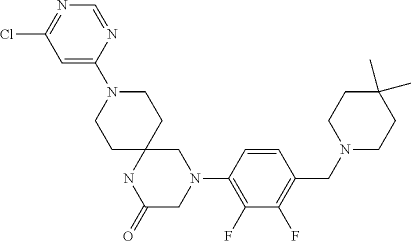 [SMILES: CC1(C)CCN(Cc2ccc(N3CC(=O)NC4(CCN(c5cc(Cl)ncn5)CC4)C3)c(F)c2F)CC1] | 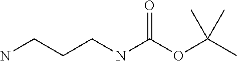 |
| | | 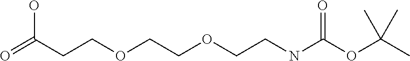 [SMILES: CC(C)(C)OC(=O)NCCOCCOCCC(=O)O] | 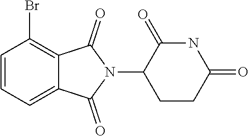 [SMILES: O=C1CCC(N2C(=O)c3cccc(Br)c3C2=O)C(=O)N1] |
| 106 | 3 | 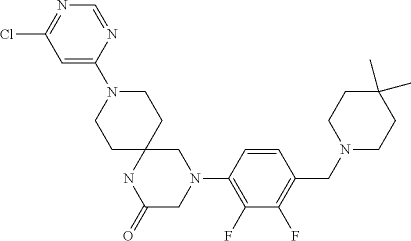 [SMILES: CC1(C)CCN(Cc2ccc(N3CC(=O)NC4(CCN(c5cc(Cl)ncn5)CC4)C3)c(F)c2F)CC1] | 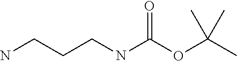 |
| | | 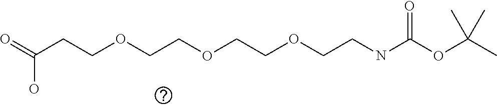 [SMILES: CC(C)(C)OC(=O)NCCOCCOCCOCCC(=O)O] | 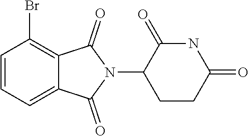 [SMILES: O=C1CCC(N2C(=O)c3cccc(Br)c3C2=O)C(=O)N1] |
| 107 | 3 | 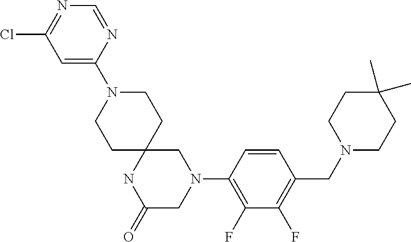 [SMILES: CC1(C)CCN(Cc2ccc(N3CC(=O)NC4(CCN(c5cc(Cl)ncn5)CC4)C3)c(F)c2F)CC1] | 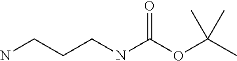 |
| | | 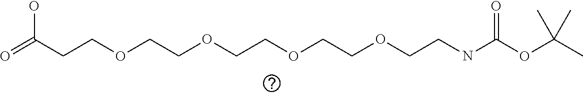 [SMILES: CC(C)(C)OC(=O)NCCOCCOCCOCCOCCC(=O)O] | 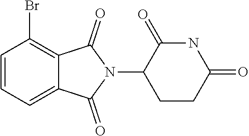 [SMILES: O=C1CCC(N2C(=O)c3cccc(Br)c3C2=O)C(=O)N1] |
| 108 | 3 | 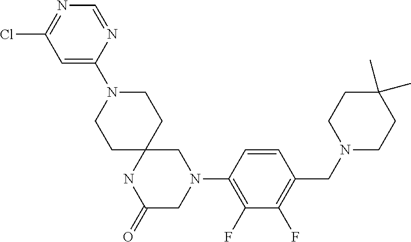 [SMILES: CC1(C)CCN(Cc2ccc(N3CC(=O)NC4(CCN(c5cc(Cl)ncn5)CC4)C3)c(F)c2F)CC1] | 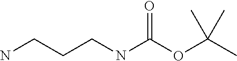 |
| | | 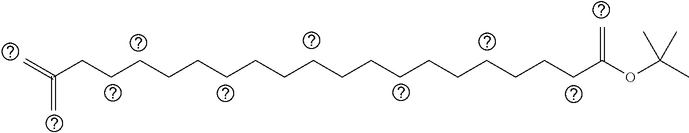 | 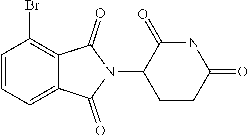 [SMILES: O=C1CCC(N2C(=O)c3cccc(Br)c3C2=O)C(=O)N1] |
| 109 | 3 | 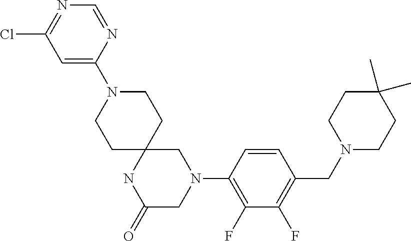 [SMILES: CC1(C)CCN(Cc2ccc(N3CC(=O)NC4(CCN(c5cc(Cl)ncn5)CC4)C3)c(F)c2F)CC1] | 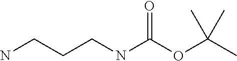 |
| | | 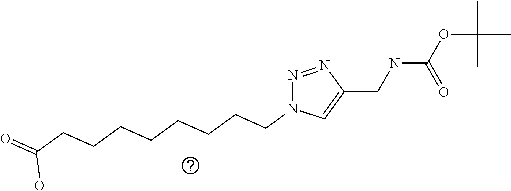 [SMILES: CC(C)(C)OC(=O)NCc1cn(CCCCCCCCC(=O)O)nn1] | 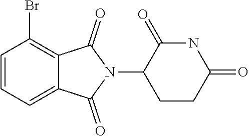 [SMILES: O=C1CCC(N2C(=O)c3cccc(Br)c3C2=O)C(=O)N1] |
| 110 | 3 | 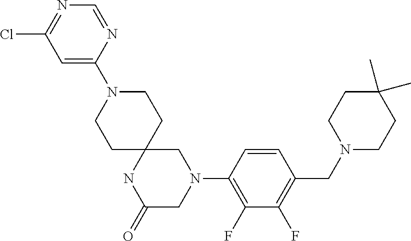 [SMILES: CC1(C)CCN(Cc2ccc(N3CC(=O)NC4(CCN(c5cc(Cl)ncn5)CC4)C3)c(F)c2F)CC1] | 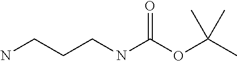 |
| | | 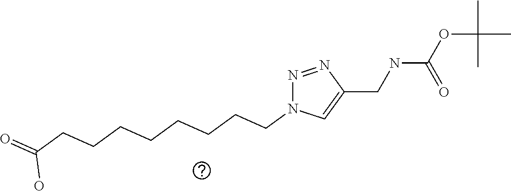 [SMILES: CC(C)(C)OC(=O)NCc1cn(CCCCCCCCC(=O)O)nn1] | 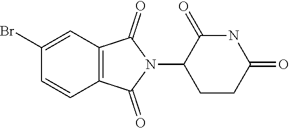 [SMILES: O=C1CCC(N2C(=O)c3ccc(Br)cc3C2=O)C(=O)N1] |
| 111 | 3 | 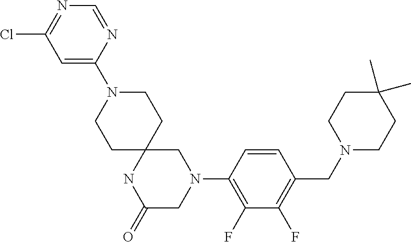 [SMILES: CC1(C)CCN(Cc2ccc(N3CC(=O)NC4(CCN(c5cc(Cl)ncn5)CC4)C3)c(F)c2F)CC1] | 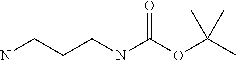 |
| | | 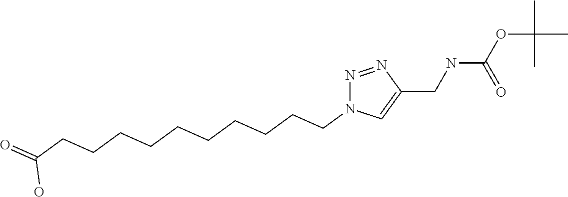 [SMILES: CC(C)(C)OC(=O)NCc1cn(CCCCCCCCCCC(=O)O)nn1] | 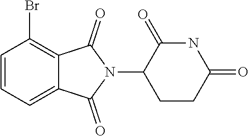 [SMILES: O=C1CCC(N2C(=O)c3cccc(Br)c3C2=O)C(=O)N1] |
| 112 | 3 | 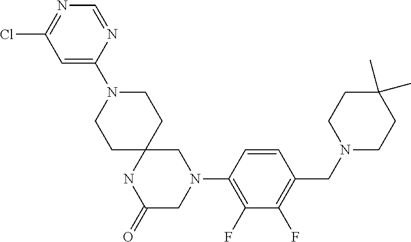 [SMILES: CC1(C)CCN(Cc2ccc(N3CC(=O)NC4(CCN(c5cc(Cl)ncn5)CC4)C3)c(F)c2F)CC1] | 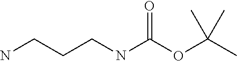 |
| | | 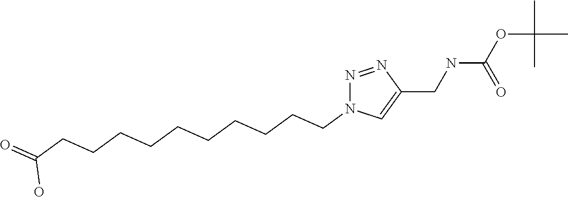 [SMILES: CC(C)(C)OC(=O)NCc1cn(CCCCCCCCCCC(=O)O)nn1] | 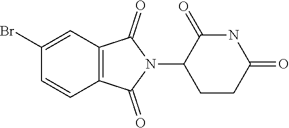 [SMILES: O=C1CCC(N2C(=O)c3ccc(Br)cc3C2=O)C(=O)N1] |
| 113 | 3 | 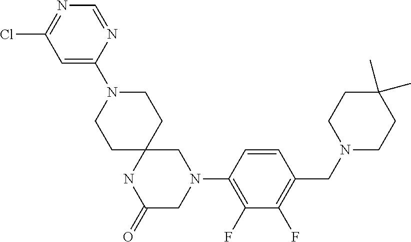 [SMILES: CC1(C)CCN(Cc2ccc(N3CC(=O)NC4(CCN(c5cc(Cl)ncn5)CC4)C3)c(F)c2F)CC1] | 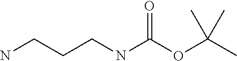 |
| | | 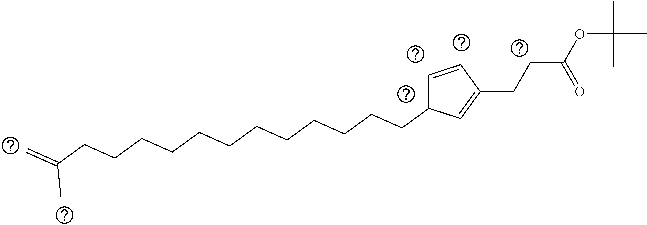 [SMILES: C=C(C)CCCCCCCCCCCCC1C=CC(CCC(=O)OC(C)(C)C)=C1] | 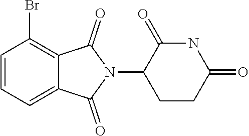 [SMILES: O=C1CCC(N2C(=O)c3cccc(Br)c3C2=O)C(=O)N1] |
| 114 | 3 | 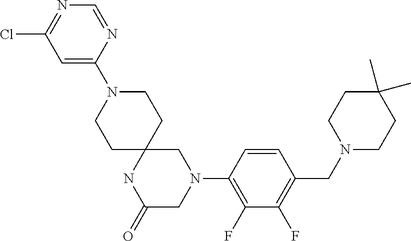 [SMILES: CC1(C)CCN(Cc2ccc(N3CC(=O)NC4(CCN(c5cc(Cl)ncn5)CC4)C3)c(F)c2F)CC1] | 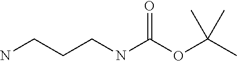 |
| | | 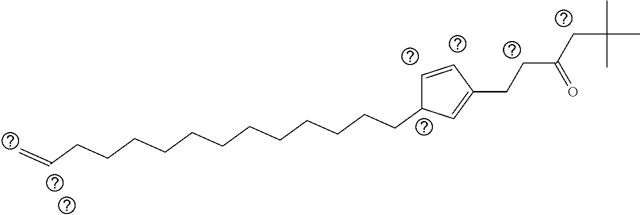 [SMILES: C=CCCCCCCCCCCCCC1C=CC(CCC(=O)CC(C)(C)C)=C1] | 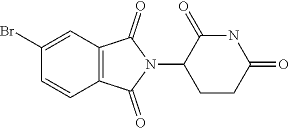 [SMILES: O=C1CCC(N2C(=O)c3ccc(Br)cc3C2=O)C(=O)N1] |
| 115 | 3 | 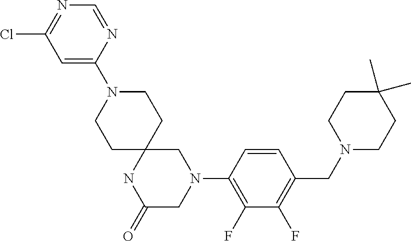 [SMILES: CC1(C)CCN(Cc2ccc(N3CC(=O)NC4(CCN(c5cc(Cl)ncn5)CC4)C3)c(F)c2F)CC1] | 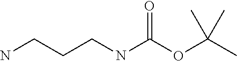 |
| | | 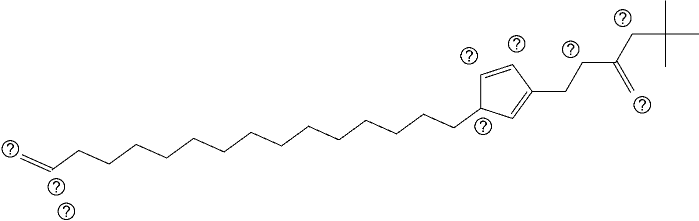 [SMILES: C=CCCCCCCCCCCCCCCC1C=CC(CCC(=C)CC(C)(C)C)=C1] | 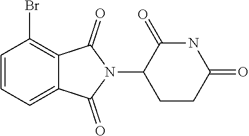 [SMILES: O=C1CCC(N2C(=O)c3cccc(Br)c3C2=O)C(=O)N1] |
| 116 | 3 | 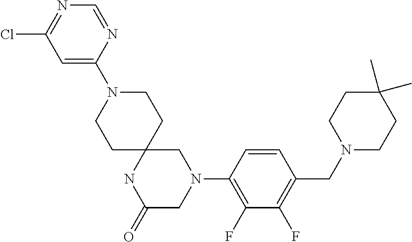 [SMILES: CC1(C)CCN(Cc2ccc(N3CC(=O)NC4(CCN(c5cc(Cl)ncn5)CC4)C3)c(F)c2F)CC1] | 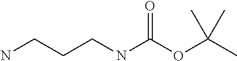 |
| | | 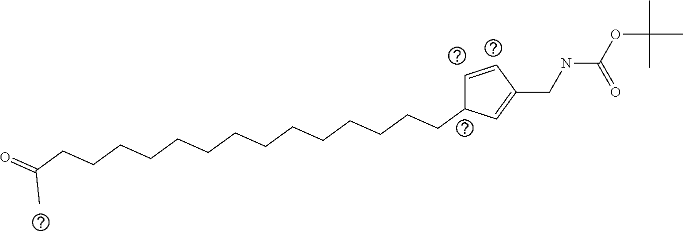 [SMILES: CC(=O)CCCCCCCCCCCCCCC1C=CC(CNC(=O)OC(C)(C)C)=C1] | 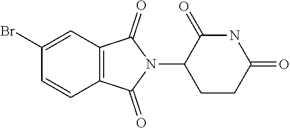 [SMILES: O=C1CCC(N2C(=O)c3ccc(Br)cc3C2=O)C(=O)N1] |
| 117 | 3 | 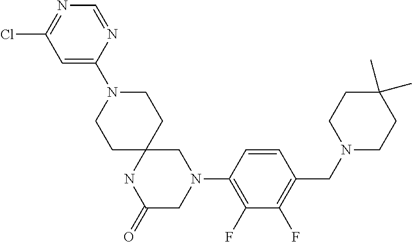 [SMILES: CC1(C)CCN(Cc2ccc(N3CC(=O)NC4(CCN(c5cc(Cl)ncn5)CC4)C3)c(F)c2F)CC1] | 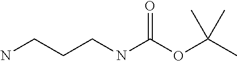 |
| | | 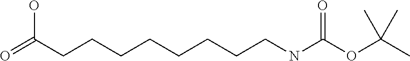 [SMILES: CC(C)(C)OC(=O)NCCCCCCCCC(=O)O] | 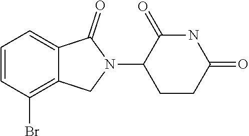 [SMILES: O=C1CCC(N2Cc3c(Br)cccc3C2=O)C(=O)N1] |
| 118 | 3 | 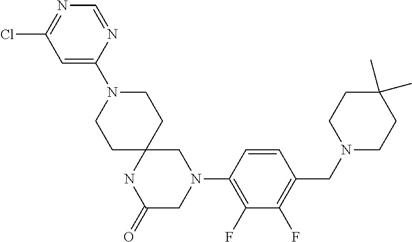 [SMILES: CC1(C)CCN(Cc2ccc(N3CC(=O)NC4(CCN(c5cc(Cl)ncn5)CC4)C3)c(F)c2F)CC1] | 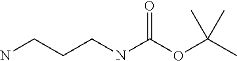 |
| | | 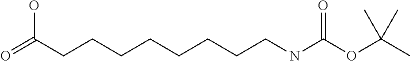 [SMILES: CC(C)(C)OC(=O)NCCCCCCCCC(=O)O] | 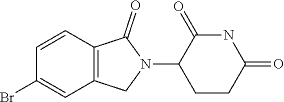 [SMILES: O=C1CCC(N2Cc3cc(Br)ccc3C2=O)C(=O)N1] |
| 119 | 3 | 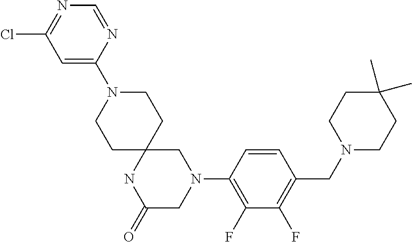 [SMILES: CC1(C)CCN(Cc2ccc(N3CC(=O)NC4(CCN(c5cc(Cl)ncn5)CC4)C3)c(F)c2F)CC1] | 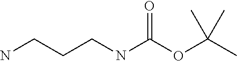 |
| | | 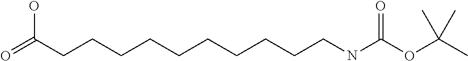 [SMILES: CC(C)(C)OC(=O)NCCCCCCCCCCC(=O)O] | 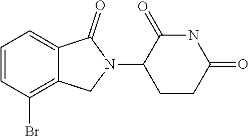 [SMILES: O=C1CCC(N2Cc3c(Br)cccc3C2=O)C(=O)N1] |
| 120 | 3 | 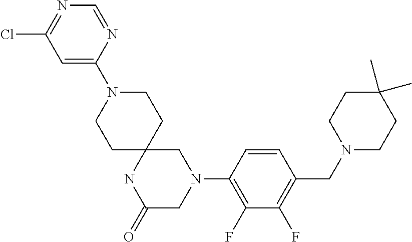 [SMILES: CC1(C)CCN(Cc2ccc(N3CC(=O)NC4(CCN(c5cc(Cl)ncn5)CC4)C3)c(F)c2F)CC1] | 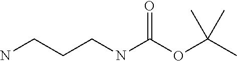 |
| | | 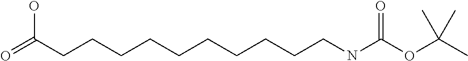 [SMILES: CC(C)(C)OC(=O)NCCCCCCCCCCC(=O)O] | 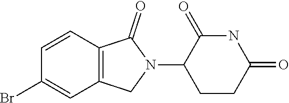 [SMILES: O=C1CCC(N2Cc3cc(Br)ccc3C2=O)C(=O)N1] |
| 121 | 3 | 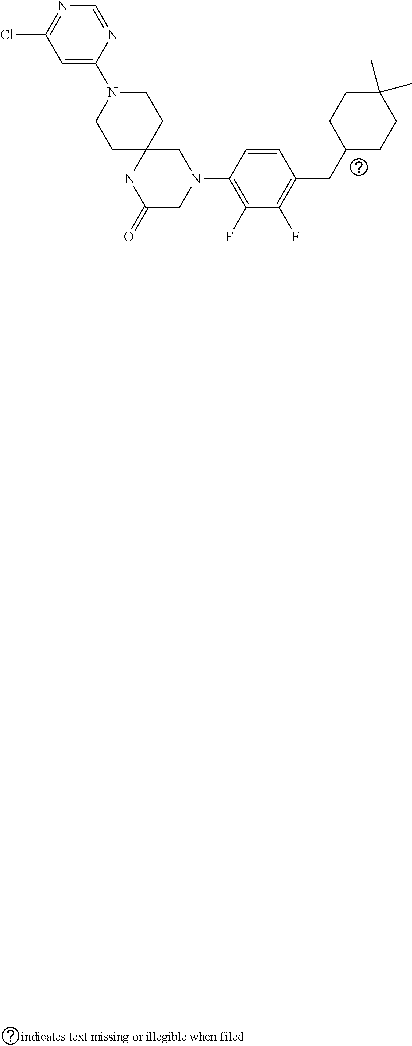 [SMILES: CC1(C)CCC(Cc2ccc(N3CC(=O)NC4(CCN(c5cc(Cl)ncn5)CC4)C3)c(F)c2F)CC1] | 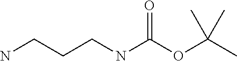 |
| | | 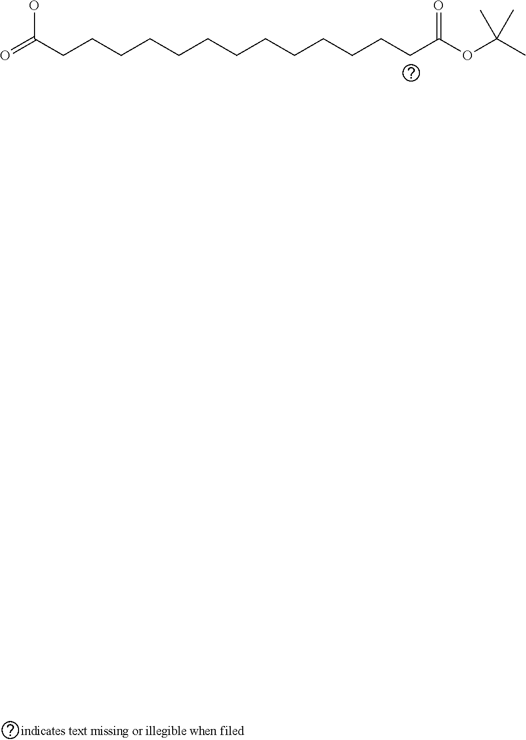 [SMILES: CC(C)(C)OC(=O)CCCCCCCCCCCCCC(=O)O] | 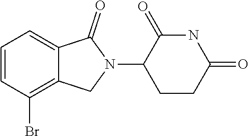 [SMILES: O=C1CCC(N2Cc3c(Br)cccc3C2=O)C(=O)N1] |
| 122 | 3 | 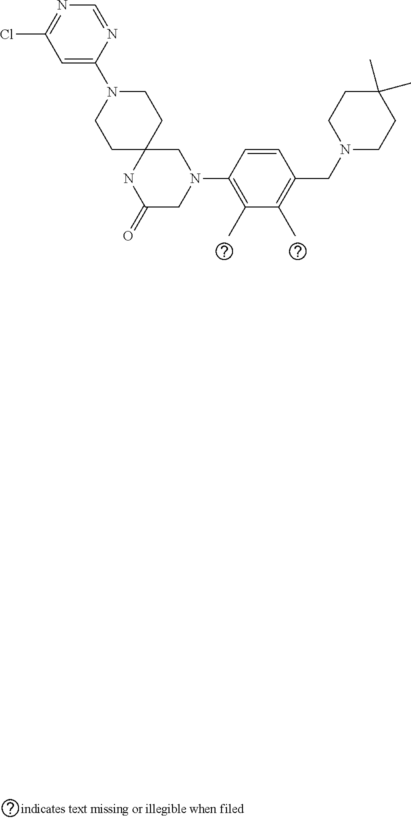 [SMILES: Cc1c(CN2CCC(C)(C)CC2)ccc(N2CC(=O)NC3(CCN(c4cc(Cl)ncn4)CC3)C2)c1C] | 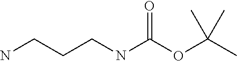 |
| | | 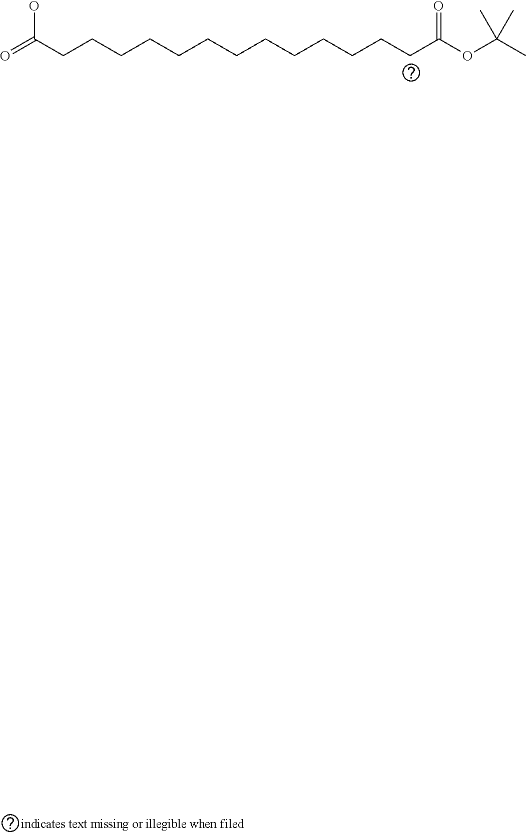 [SMILES: CC(C)(C)OC(=O)CCCCCCCCCCCCCC(=O)O] | 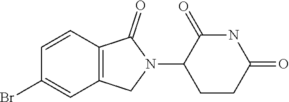 [SMILES: O=C1CCC(N2Cc3cc(Br)ccc3C2=O)C(=O)N1] |
| 123 | 3 | 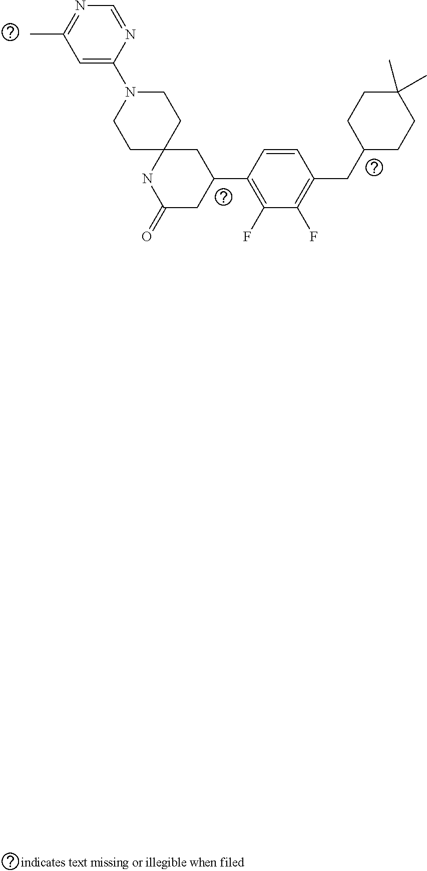 | 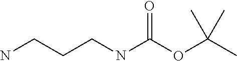 |
| | | 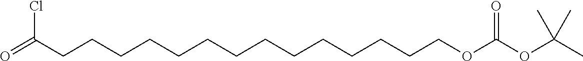 [SMILES: CC(C)(C)OC(=O)OCCCCCCCCCCCCCCC(=O)Cl] | 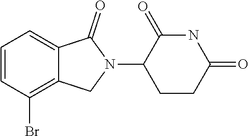 [SMILES: O=C1CCC(N2Cc3c(Br)cccc3C2=O)C(=O)N1] |
| 124 | 3 | 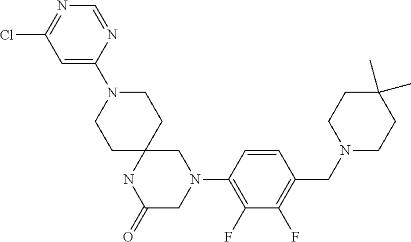 [SMILES: CC1(C)CCN(Cc2ccc(N3CC(=O)NC4(CCN(c5cc(Cl)ncn5)CC4)C3)c(F)c2F)CC1] | 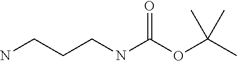 |
| | | 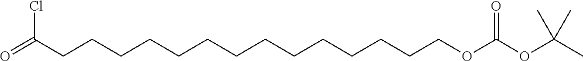 [SMILES: CC(C)(C)OC(=O)OCCCCCCCCCCCCCCC(=O)Cl] | 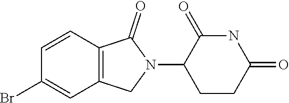 [SMILES: O=C1CCC(N2Cc3cc(Br)ccc3C2=O)C(=O)N1] |
| 125 | 3 | 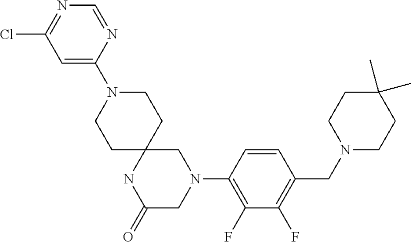 [SMILES: CC1(C)CCN(Cc2ccc(N3CC(=O)NC4(CCN(c5cc(Cl)ncn5)CC4)C3)c(F)c2F)CC1] | 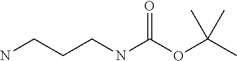 |
| | | 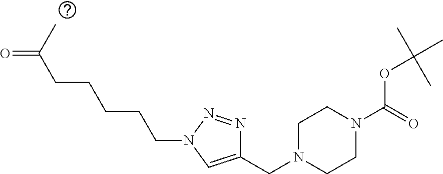 [SMILES: CC(=O)CCCCCn1cc(CN2CCN(C(=O)OC(C)(C)C)CC2)nn1] | 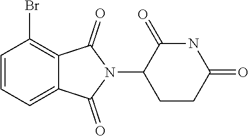 [SMILES: O=C1CCC(N2C(=O)c3cccc(Br)c3C2=O)C(=O)N1] |
| 126 | 3 | 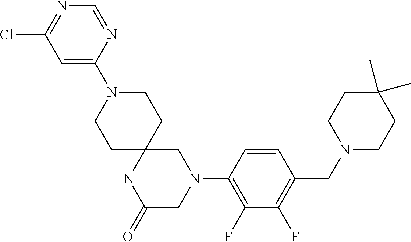 [SMILES: CC1(C)CCN(Cc2ccc(N3CC(=O)NC4(CCN(c5cc(Cl)ncn5)CC4)C3)c(F)c2F)CC1] | 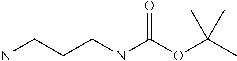 |
| | | 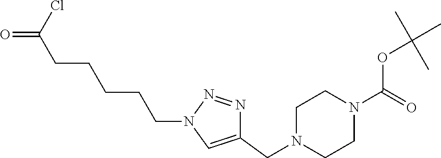 [SMILES: CC(C)(C)OC(=O)N1CCN(Cc2cn(CCCCCC(=O)Cl)nn2)CC1] | 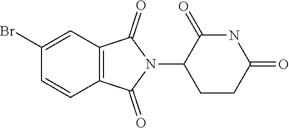 [SMILES: O=C1CCC(N2C(=O)c3ccc(Br)cc3C2=O)C(=O)N1] |
| 127 | 3 | 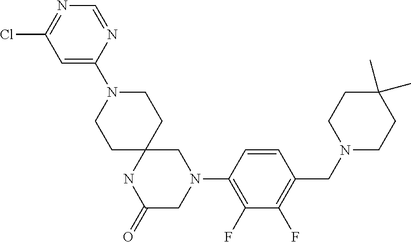 [SMILES: CC1(C)CCN(Cc2ccc(N3CC(=O)NC4(CCN(c5cc(Cl)ncn5)CC4)C3)c(F)c2F)CC1] | 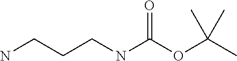 |
| | | 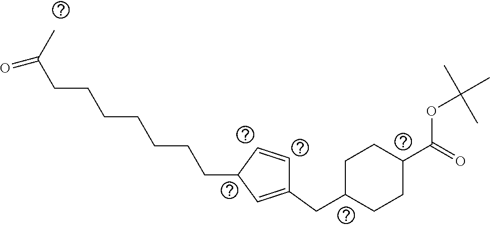 [SMILES: CC(=O)CCCCCCCC1C=CC(CC2CCC(C(=O)OC(C)(C)C)CC2)=C1] | 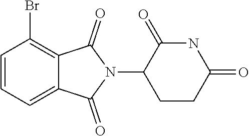 [SMILES: O=C1CCC(N2C(=O)c3cccc(Br)c3C2=O)C(=O)N1] |
| 128 | 3 | 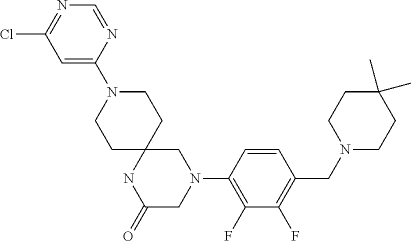 [SMILES: CC1(C)CCN(Cc2ccc(N3CC(=O)NC4(CCN(c5cc(Cl)ncn5)CC4)C3)c(F)c2F)CC1] | 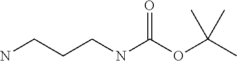 |
| | | 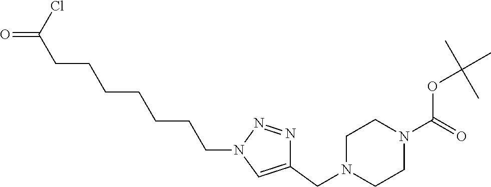 [SMILES: CC(C)(C)OC(=O)N1CCN(Cc2cn(CCCCCCCC(=O)Cl)nn2)CC1] | 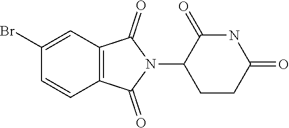 [SMILES: O=C1CCC(N2C(=O)c3ccc(Br)cc3C2=O)C(=O)N1] |
| 129 | 3 | 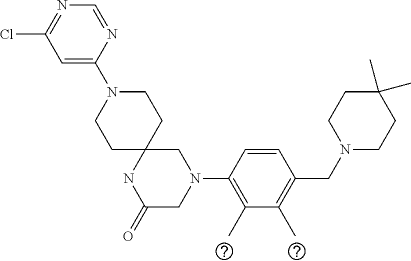 [SMILES: Cc1c(CN2CCC(C)(C)CC2)ccc(N2CC(=O)NC3(CCN(c4cc(Cl)ncn4)CC3)C2)c1C] | 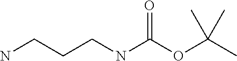 |
| | | 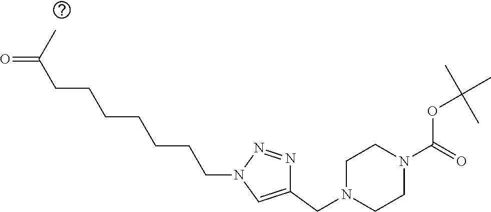 [SMILES: CC(=O)CCCCCCCn1cc(CN2CCN(C(=O)OC(C)(C)C)CC2)nn1] | 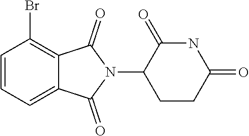 [SMILES: O=C1CCC(N2C(=O)c3cccc(Br)c3C2=O)C(=O)N1] |
| 130 | 3 | 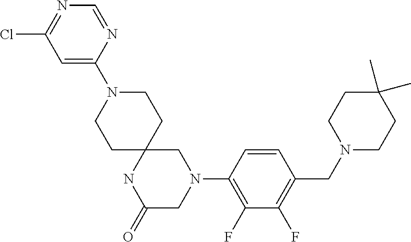 [SMILES: CC1(C)CCN(Cc2ccc(N3CC(=O)NC4(CCN(c5cc(Cl)ncn5)CC4)C3)c(F)c2F)CC1] | 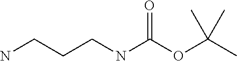 |
| | | 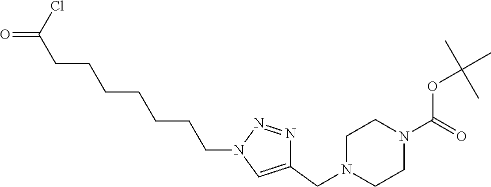 [SMILES: CC(C)(C)OC(=O)N1CCN(Cc2cn(CCCCCCCC(=O)Cl)nn2)CC1] | 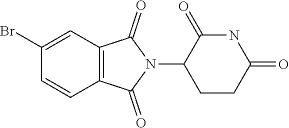 [SMILES: O=C1CCC(N2C(=O)c3ccc(Br)cc3C2=O)C(=O)N1] |
| 131 | 3 | 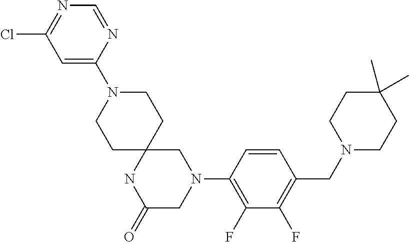 [SMILES: CC1(C)CCN(Cc2ccc(N3CC(=O)NC4(CCN(c5cc(Cl)ncn5)CC4)C3)c(F)c2F)CC1] | 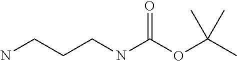 |
| | | 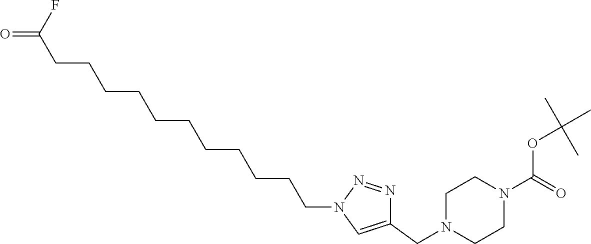 [SMILES: CC(C)(C)OC(=O)N1CCN(Cc2cn(CCCCCCCCCCCC(=O)F)nn2)CC1] | 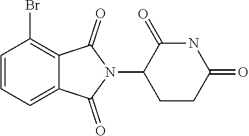 [SMILES: O=C1CCC(N2C(=O)c3cccc(Br)c3C2=O)C(=O)N1] |
| 132 | 3 | 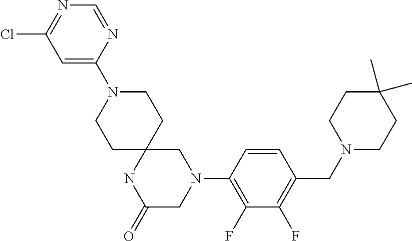 [SMILES: CC1(C)CCN(Cc2ccc(N3CC(=O)NC4(CCN(c5cc(Cl)ncn5)CC4)C3)c(F)c2F)CC1] | 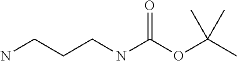 |
| | | 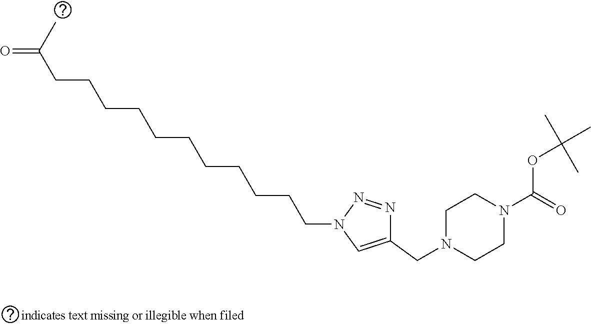 [SMILES: CC(=O)CCCCCCCCCCCn1cc(CN2CCN(C(=O)OC(C)(C)C)CC2)nn1] | 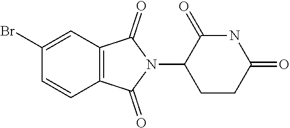 [SMILES: O=C1CCC(N2C(=O)c3ccc(Br)cc3C2=O)C(=O)N1] |
| 133 | 3 | 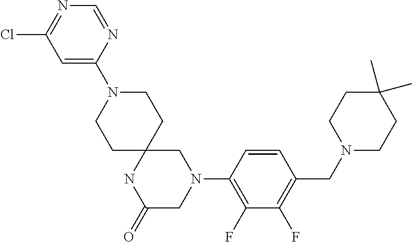 [SMILES: CC1(C)CCN(Cc2ccc(N3CC(=O)NC4(CCN(c5cc(Cl)ncn5)CC4)C3)c(F)c2F)CC1] | 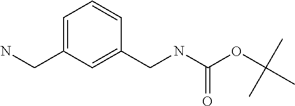 [SMILES: CC(C)(C)OC(=O)NCc1cccc(CN)c1] |
| | | 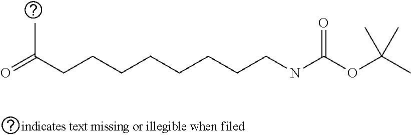 [SMILES: CC(=O)CCCCCCCCNC(=O)OC(C)(C)C] | 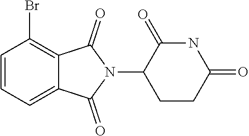 [SMILES: O=C1CCC(N2C(=O)c3cccc(Br)c3C2=O)C(=O)N1] |
| 134 | 3 | 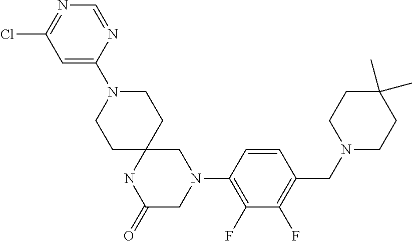 [SMILES: CC1(C)CCN(Cc2ccc(N3CC(=O)NC4(CCN(c5cc(Cl)ncn5)CC4)C3)c(F)c2F)CC1] | 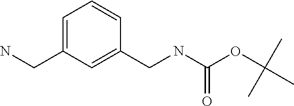 [SMILES: CC(C)(C)OC(=O)NCc1cccc(CN)c1] |
| | | 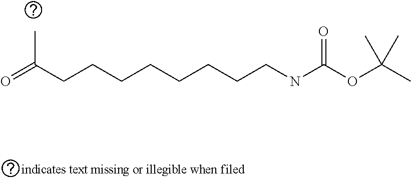 [SMILES: CC(=O)CCCCCCCCNC(=O)OC(C)(C)C] | 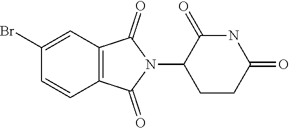 [SMILES: O=C1CCC(N2C(=O)c3ccc(Br)cc3C2=O)C(=O)N1] |
| 135 | 3 | 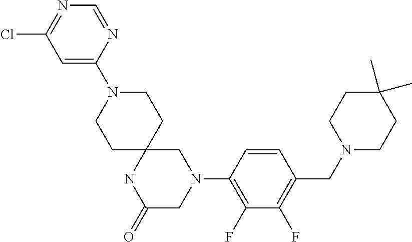 [SMILES: CC1(C)CCN(Cc2ccc(N3CC(=O)NC4(CCN(c5cc(Cl)ncn5)CC4)C3)c(F)c2F)CC1] | 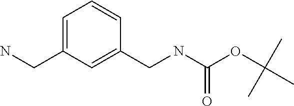 [SMILES: CC(C)(C)OC(=O)NCc1cccc(CN)c1] |
| | | 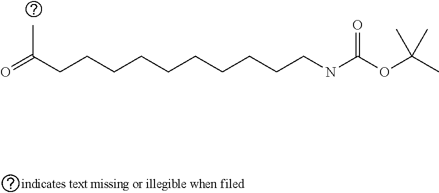 [SMILES: CC(=O)CCCCCCCCCCNC(=O)OC(C)(C)C] | 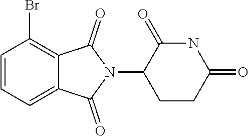 [SMILES: O=C1CCC(N2C(=O)c3cccc(Br)c3C2=O)C(=O)N1] |
| 136 | 3 | 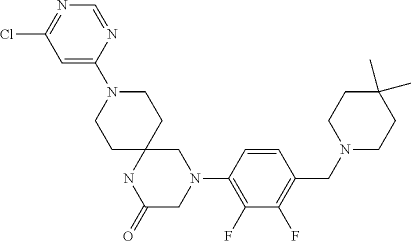 [SMILES: CC1(C)CCN(Cc2ccc(N3CC(=O)NC4(CCN(c5cc(Cl)ncn5)CC4)C3)c(F)c2F)CC1] | 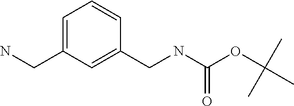 [SMILES: CC(C)(C)OC(=O)NCc1cccc(CN)c1] |
| | | 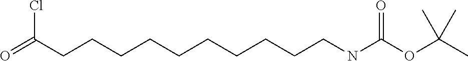 [SMILES: CC(C)(C)OC(=O)NCCCCCCCCCCC(=O)Cl] | 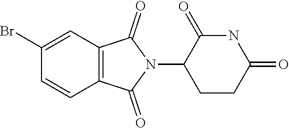 [SMILES: O=C1CCC(N2C(=O)c3ccc(Br)cc3C2=O)C(=O)N1] |
| 137 | 3 | 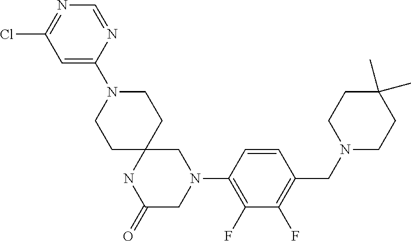 [SMILES: CC1(C)CCN(Cc2ccc(N3CC(=O)NC4(CCN(c5cc(Cl)ncn5)CC4)C3)c(F)c2F)CC1] | 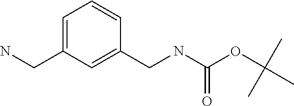 [SMILES: CC(C)(C)OC(=O)NCc1cccc(CN)c1] |
| | | 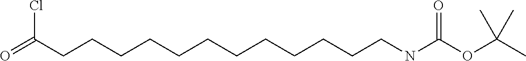 [SMILES: CC(C)(C)OC(=O)NCCCCCCCCCCCCC(=O)Cl] | 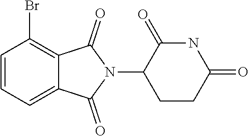 [SMILES: O=C1CCC(N2C(=O)c3cccc(Br)c3C2=O)C(=O)N1] |
| 138 | 3 | 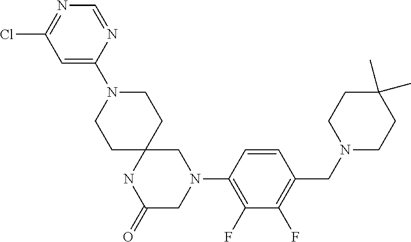 [SMILES: CC1(C)CCN(Cc2ccc(N3CC(=O)NC4(CCN(c5cc(Cl)ncn5)CC4)C3)c(F)c2F)CC1] | 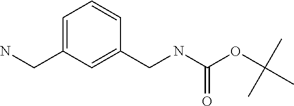 [SMILES: CC(C)(C)OC(=O)NCc1cccc(CN)c1] |
| | | 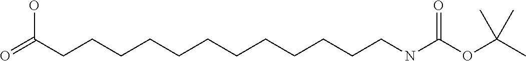 [SMILES: CC(C)(C)OC(=O)NCCCCCCCCCCCCC(=O)O] | 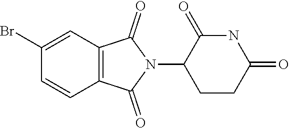 [SMILES: O=C1CCC(N2C(=O)c3ccc(Br)cc3C2=O)C(=O)N1] |
| 139 | 3 | 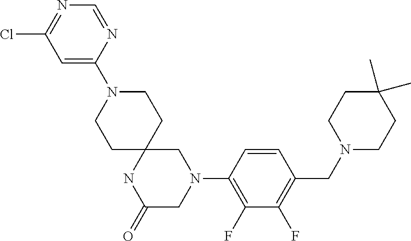 [SMILES: CC1(C)CCN(Cc2ccc(N3CC(=O)NC4(CCN(c5cc(Cl)ncn5)CC4)C3)c(F)c2F)CC1] | 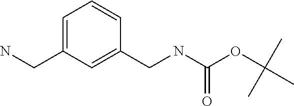 [SMILES: CC(C)(C)OC(=O)NCc1cccc(CN)c1] |
| | | 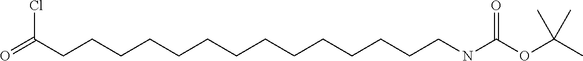 [SMILES: CC(C)(C)OC(=O)NCCCCCCCCCCCCCCC(=O)Cl] | 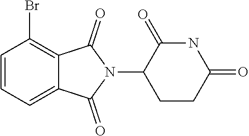 [SMILES: O=C1CCC(N2C(=O)c3cccc(Br)c3C2=O)C(=O)N1] |
| 143 | 3 | 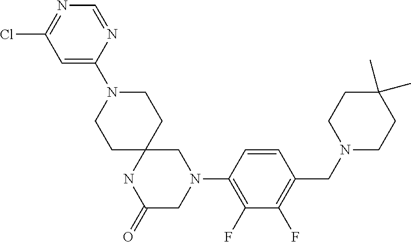 [SMILES: CC1(C)CCN(Cc2ccc(N3CC(=O)NC4(CCN(c5cc(Cl)ncn5)CC4)C3)c(F)c2F)CC1] | 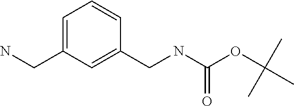 [SMILES: CC(C)(C)OC(=O)NCc1cccc(CN)c1] |
| | | 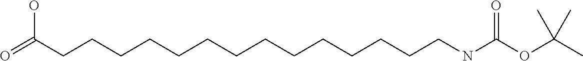 [SMILES: CC(C)(C)OC(=O)NCCCCCCCCCCCCCCC(=O)O] | 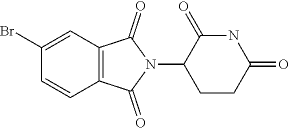 [SMILES: O=C1CCC(N2C(=O)c3ccc(Br)cc3C2=O)C(=O)N1] |
| 141 | 3 | 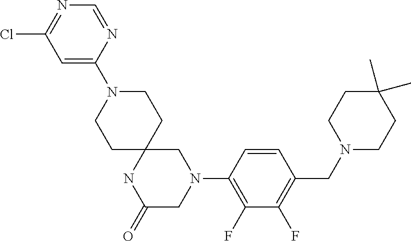 [SMILES: CC1(C)CCN(Cc2ccc(N3CC(=O)NC4(CCN(c5cc(Cl)ncn5)CC4)C3)c(F)c2F)CC1] | 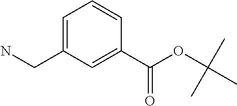 |
| | | 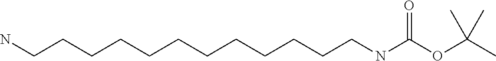 [SMILES: CC(C)(C)OC(=O)NCCCCCCCCCCCCN] | 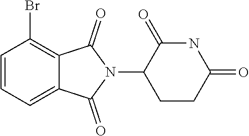 [SMILES: O=C1CCC(N2C(=O)c3cccc(Br)c3C2=O)C(=O)N1] |
| 142 | 3 | 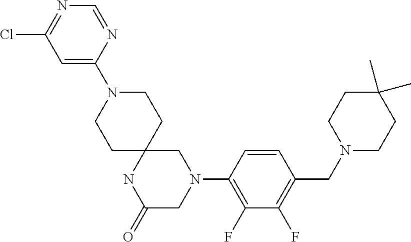 [SMILES: CC1(C)CCN(Cc2ccc(N3CC(=O)NC4(CCN(c5cc(Cl)ncn5)CC4)C3)c(F)c2F)CC1] | 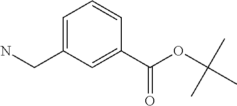 |
| | | 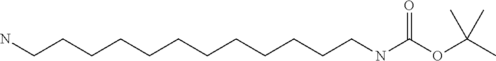 [SMILES: CC(C)(C)OC(=O)NCCCCCCCCCCCCN] | 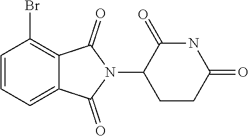 [SMILES: O=C1CCC(N2C(=O)c3cccc(Br)c3C2=O)C(=O)N1] |
| 143 | 3 | 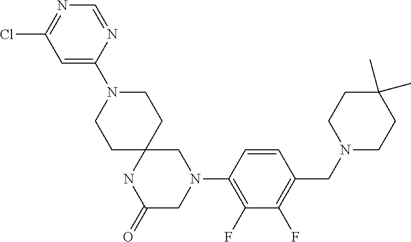 [SMILES: CC1(C)CCN(Cc2ccc(N3CC(=O)NC4(CCN(c5cc(Cl)ncn5)CC4)C3)c(F)c2F)CC1] | 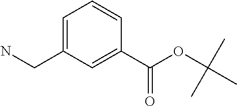 |
| | | 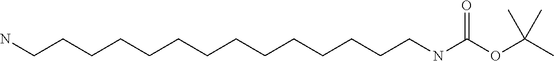 [SMILES: CC(C)(C)OC(=O)NCCCCCCCCCCCCCCN] | 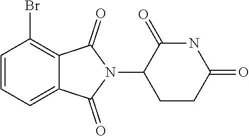 [SMILES: O=C1CCC(N2C(=O)c3cccc(Br)c3C2=O)C(=O)N1] |
| 144 | 3 | 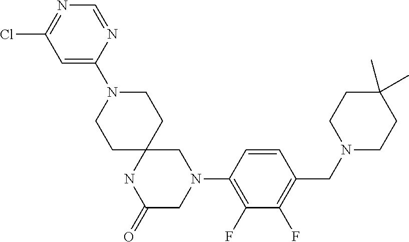 [SMILES: CC1(C)CCN(Cc2ccc(N3CC(=O)NC4(CCN(c5cc(Cl)ncn5)CC4)C3)c(F)c2F)CC1] | 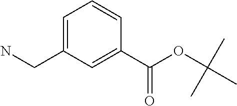 |
| | | 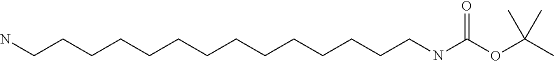 [SMILES: CC(C)(C)OC(=O)NCCCCCCCCCCCCCCN] | 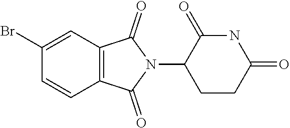 [SMILES: O=C1CCC(N2C(=O)c3ccc(Br)cc3C2=O)C(=O)N1] |
| 145 | 3 | 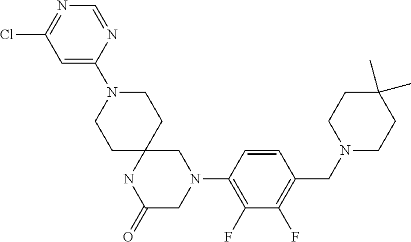 [SMILES: CC1(C)CCN(Cc2ccc(N3CC(=O)NC4(CCN(c5cc(Cl)ncn5)CC4)C3)c(F)c2F)CC1] | 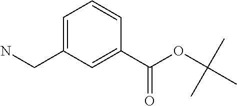 |
| | | 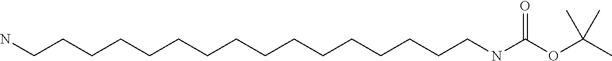 [SMILES: CC(C)(C)OC(=O)NCCCCCCCCCCCCCCCCN] | 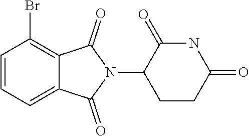 [SMILES: O=C1CCC(N2C(=O)c3cccc(Br)c3C2=O)C(=O)N1] |
| 146 | 3 | 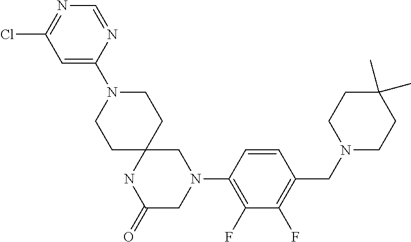 [SMILES: CC1(C)CCN(Cc2ccc(N3CC(=O)NC4(CCN(c5cc(Cl)ncn5)CC4)C3)c(F)c2F)CC1] | 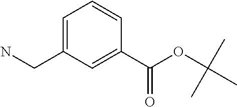 |
| | | 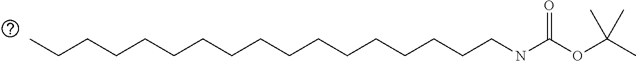 [SMILES: CCCCCCCCCCCCCCCCCNC(=O)OC(C)(C)C] | 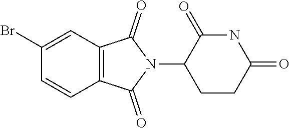 [SMILES: O=C1CCC(N2C(=O)c3ccc(Br)cc3C2=O)C(=O)N1] |
| 147 | 3 | 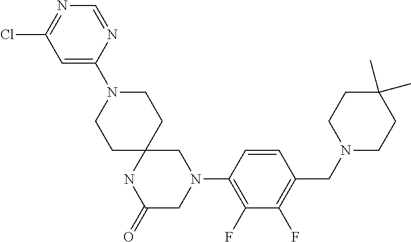 [SMILES: CC1(C)CCN(Cc2ccc(N3CC(=O)NC4(CCN(c5cc(Cl)ncn5)CC4)C3)c(F)c2F)CC1] | 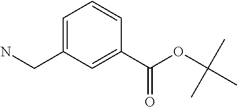 |
| | | 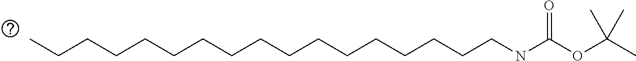 [SMILES: CCCCCCCCCCCCCCCCCNC(=O)OC(C)(C)C] | 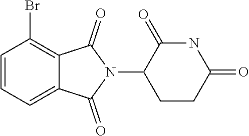 [SMILES: O=C1CCC(N2C(=O)c3cccc(Br)c3C2=O)C(=O)N1] |
| 148 | 3 | 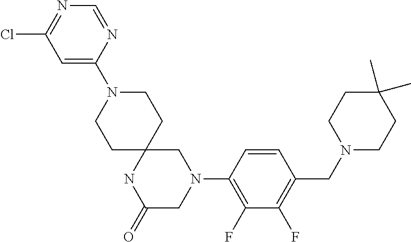 [SMILES: CC1(C)CCN(Cc2ccc(N3CC(=O)NC4(CCN(c5cc(Cl)ncn5)CC4)C3)c(F)c2F)CC1] | 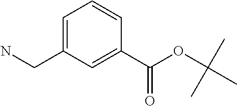 |
| | | 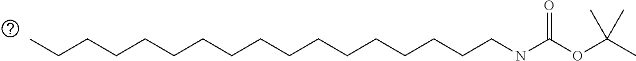 [SMILES: CCCCCCCCCCCCCCCCCNC(=O)OC(C)(C)C] | 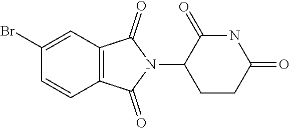 [SMILES: O=C1CCC(N2C(=O)c3ccc(Br)cc3C2=O)C(=O)N1] |
| 149 | 3 | 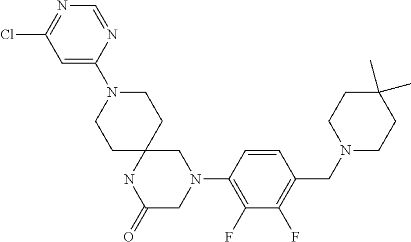 [SMILES: CC1(C)CCN(Cc2ccc(N3CC(=O)NC4(CCN(c5cc(Cl)ncn5)CC4)C3)c(F)c2F)CC1] | 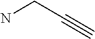 |
| | | 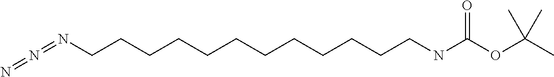 | 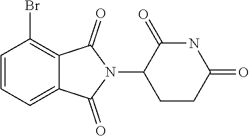 [SMILES: O=C1CCC(N2C(=O)c3cccc(Br)c3C2=O)C(=O)N1] |
| 150 | 3 | 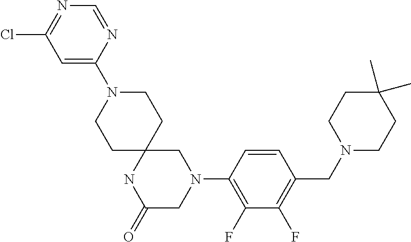 [SMILES: CC1(C)CCN(Cc2ccc(N3CC(=O)NC4(CCN(c5cc(Cl)ncn5)CC4)C3)c(F)c2F)CC1] | 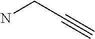 |
| | | 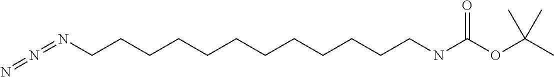 | 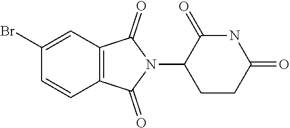 [SMILES: O=C1CCC(N2C(=O)c3ccc(Br)cc3C2=O)C(=O)N1] |
| 151 | 3 | 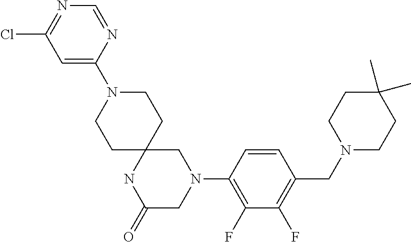 [SMILES: CC1(C)CCN(Cc2ccc(N3CC(=O)NC4(CCN(c5cc(Cl)ncn5)CC4)C3)c(F)c2F)CC1] | 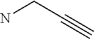 |
| | | 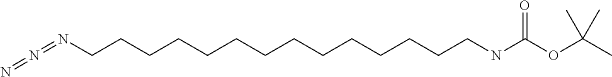 | 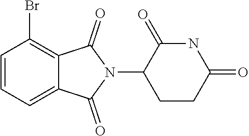 [SMILES: O=C1CCC(N2C(=O)c3cccc(Br)c3C2=O)C(=O)N1] |
| 152 | 3 | 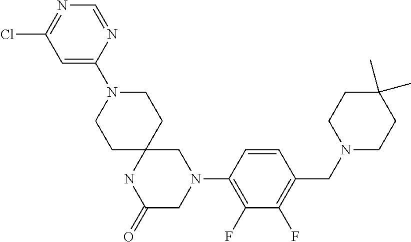 [SMILES: CC1(C)CCN(Cc2ccc(N3CC(=O)NC4(CCN(c5cc(Cl)ncn5)CC4)C3)c(F)c2F)CC1] | 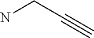 |
| | | 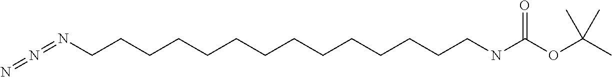 | 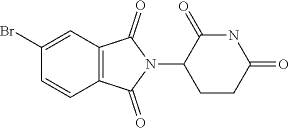 [SMILES: O=C1CCC(N2C(=O)c3ccc(Br)cc3C2=O)C(=O)N1] |
| 153 | 3 | 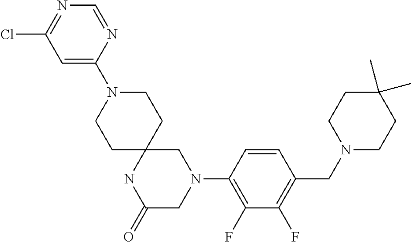 [SMILES: CC1(C)CCN(Cc2ccc(N3CC(=O)NC4(CCN(c5cc(Cl)ncn5)CC4)C3)c(F)c2F)CC1] | 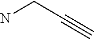 |
| | | 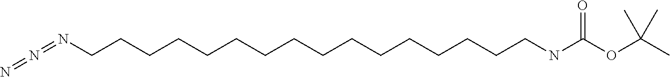 | 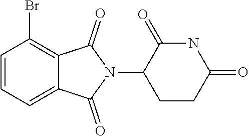 [SMILES: O=C1CCC(N2C(=O)c3cccc(Br)c3C2=O)C(=O)N1] |
| 154 | 3 | 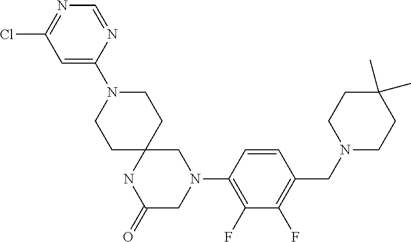 [SMILES: CC1(C)CCN(Cc2ccc(N3CC(=O)NC4(CCN(c5cc(Cl)ncn5)CC4)C3)c(F)c2F)CC1] | 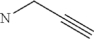 |
| | | 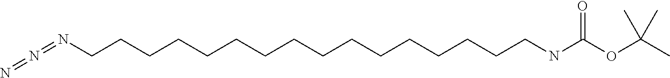 | 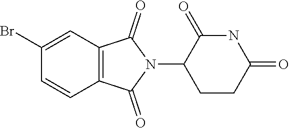 [SMILES: O=C1CCC(N2C(=O)c3ccc(Br)cc3C2=O)C(=O)N1] |
| 155 | 3 | 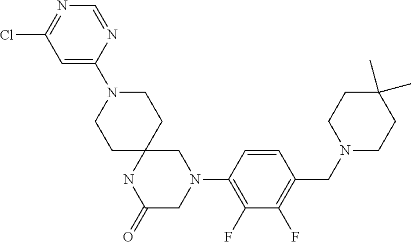 [SMILES: CC1(C)CCN(Cc2ccc(N3CC(=O)NC4(CCN(c5cc(Cl)ncn5)CC4)C3)c(F)c2F)CC1] | 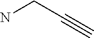 |
| | | 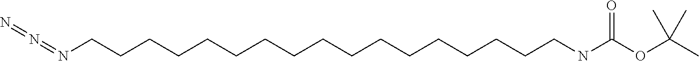 | 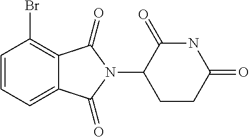 [SMILES: O=C1CCC(N2C(=O)c3cccc(Br)c3C2=O)C(=O)N1] |
| 156 | 3 | 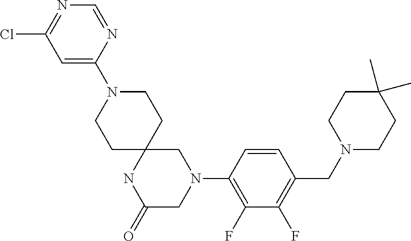 [SMILES: CC1(C)CCN(Cc2ccc(N3CC(=O)NC4(CCN(c5cc(Cl)ncn5)CC4)C3)c(F)c2F)CC1] | 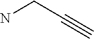 |
| | | 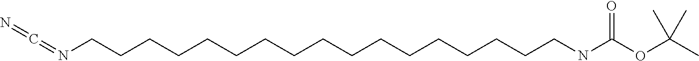 [SMILES: CC(C)(C)OC(=O)NCCCCCCCCCCCCCCCCCN=C=N] | 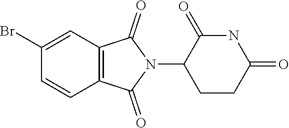 [SMILES: O=C1CCC(N2C(=O)c3ccc(Br)cc3C2=O)C(=O)N1] |
| 157 | 4 | 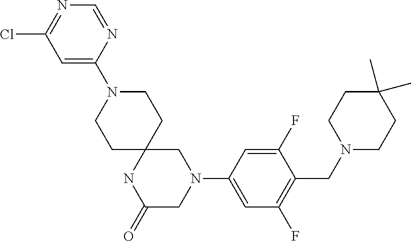 [SMILES: CC1(C)CCN(Cc2c(F)cc(N3CC(=O)NC4(CCN(c5cc(Cl)ncn5)CC4)C3)cc2F)CC1] | 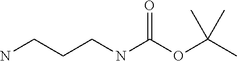 |
| | | 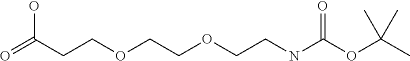 [SMILES: CC(C)(C)OC(=O)NCCOCCOCCC(=O)O] | 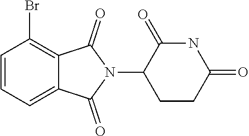 [SMILES: O=C1CCC(N2C(=O)c3cccc(Br)c3C2=O)C(=O)N1] |
| 158 | 4 | 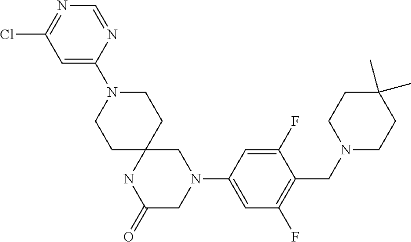 [SMILES: CC1(C)CCN(Cc2c(F)cc(N3CC(=O)NC4(CCN(c5cc(Cl)ncn5)CC4)C3)cc2F)CC1] | 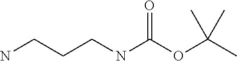 |
| | | 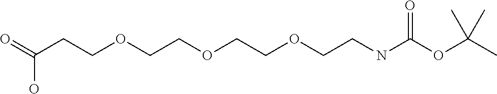 [SMILES: CC(C)(C)OC(=O)NCCOCCOCCOCCC(=O)O] | 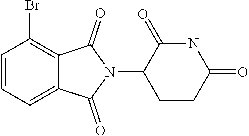 [SMILES: O=C1CCC(N2C(=O)c3cccc(Br)c3C2=O)C(=O)N1] |
| 159 | 4 | 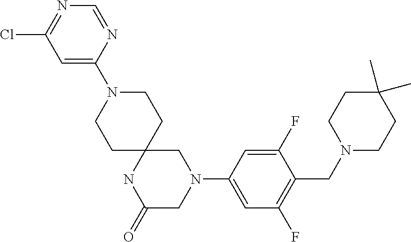 [SMILES: CC1(C)CCN(Cc2c(F)cc(N3CC(=O)NC4(CCN(c5cc(Cl)ncn5)CC4)C3)cc2F)CC1] | 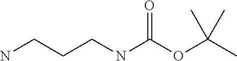 |
| | | 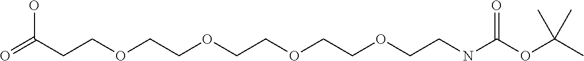 [SMILES: CC(C)(C)OC(=O)NCCOCCOCCOCCOCCC(=O)O] | 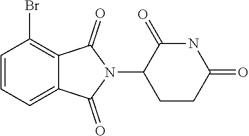 [SMILES: O=C1CCC(N2C(=O)c3cccc(Br)c3C2=O)C(=O)N1] |
| 160 | 4 | 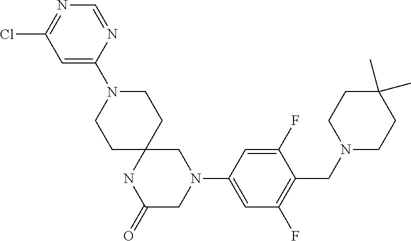 [SMILES: CC1(C)CCN(Cc2c(F)cc(N3CC(=O)NC4(CCN(c5cc(Cl)ncn5)CC4)C3)cc2F)CC1] | 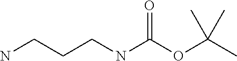 |
| | | 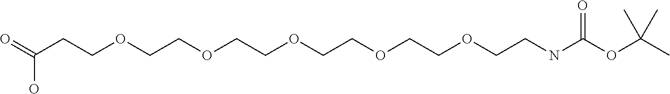 [SMILES: CC(C)(C)OC(=O)NCCOCCOCCOCCOCCOCCC(=O)O] | 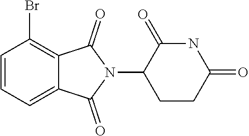 [SMILES: O=C1CCC(N2C(=O)c3cccc(Br)c3C2=O)C(=O)N1] |
| 161 | 4 | 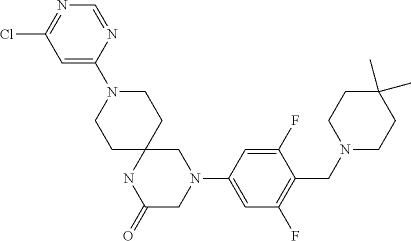 [SMILES: CC1(C)CCN(Cc2c(F)cc(N3CC(=O)NC4(CCN(c5cc(Cl)ncn5)CC4)C3)cc2F)CC1] | 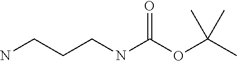 |
| | | 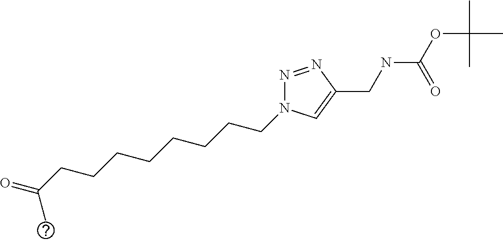 [SMILES: CC(=O)CCCCCCCCn1cc(CNC(=O)OC(C)(C)C)nn1] | 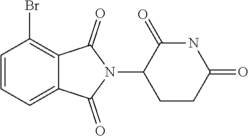 [SMILES: O=C1CCC(N2C(=O)c3cccc(Br)c3C2=O)C(=O)N1] |
| 162 | 4 | 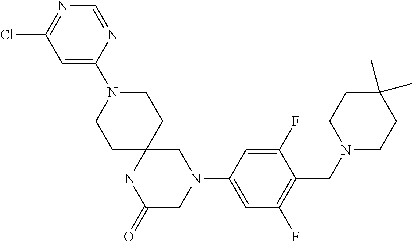 [SMILES: CC1(C)CCN(Cc2c(F)cc(N3CC(=O)NC4(CCN(c5cc(Cl)ncn5)CC4)C3)cc2F)CC1] | 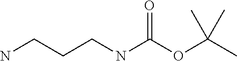 |
| | | 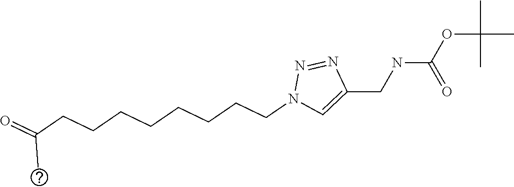 [SMILES: CC(=O)CCCCCCCCn1cc(CNC(=O)OC(C)(C)C)nn1] | 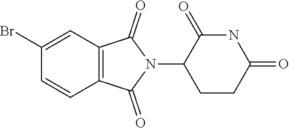 [SMILES: O=C1CCC(N2C(=O)c3ccc(Br)cc3C2=O)C(=O)N1] |
| 163 | 4 | 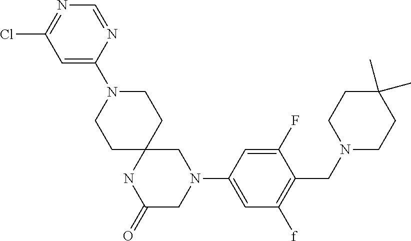 | 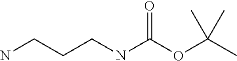 |
| | | 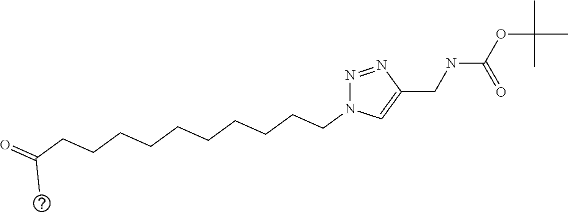 [SMILES: CC(=O)CCCCCCCCCCn1cc(CNC(=O)OC(C)(C)C)nn1] | 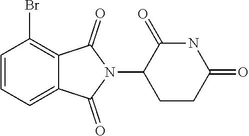 [SMILES: O=C1CCC(N2C(=O)c3cccc(Br)c3C2=O)C(=O)N1] |
| 164 | 4 | 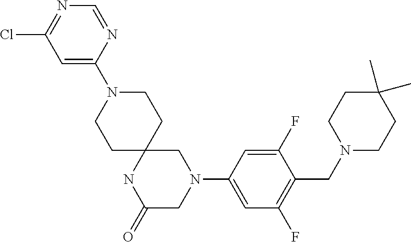 [SMILES: CC1(C)CCN(Cc2c(F)cc(N3CC(=O)NC4(CCN(c5cc(Cl)ncn5)CC4)C3)cc2F)CC1] | 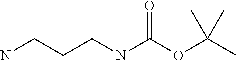 |
| | | 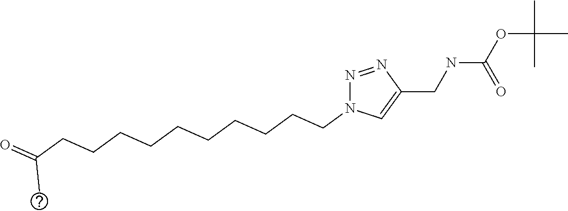 [SMILES: CC(=O)CCCCCCCCCCn1cc(CNC(=O)OC(C)(C)C)nn1] | 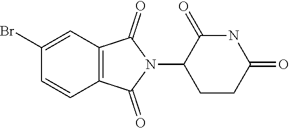 [SMILES: O=C1CCC(N2C(=O)c3ccc(Br)cc3C2=O)C(=O)N1] |
| 165 | 4 | 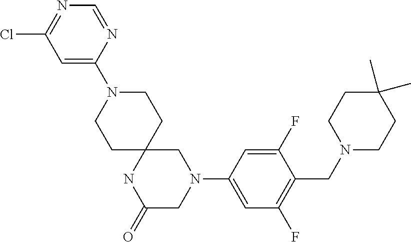 [SMILES: CC1(C)CCN(Cc2c(F)cc(N3CC(=O)NC4(CCN(c5cc(Cl)ncn5)CC4)C3)cc2F)CC1] | 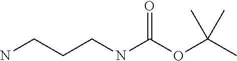 |
| | | 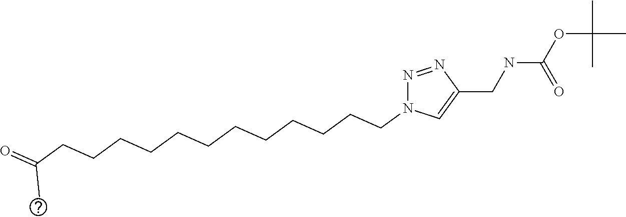 [SMILES: CC(=O)CCCCCCCCCCCCn1cc(CNC(=O)OC(C)(C)C)nn1] | 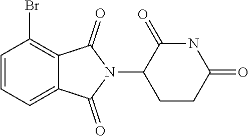 [SMILES: O=C1CCC(N2C(=O)c3cccc(Br)c3C2=O)C(=O)N1] |
| 166 | 4 | 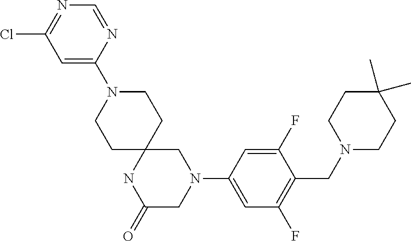 [SMILES: CC1(C)CCN(Cc2c(F)cc(N3CC(=O)NC4(CCN(c5cc(Cl)ncn5)CC4)C3)cc2F)CC1] | 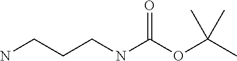 |
| | | 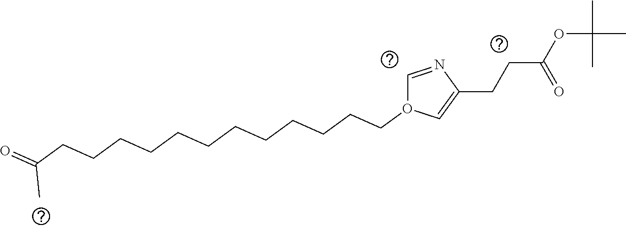 | 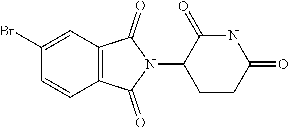 [SMILES: O=C1CCC(N2C(=O)c3ccc(Br)cc3C2=O)C(=O)N1] |
| 167 | 4 | 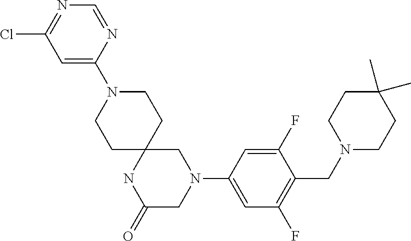 [SMILES: CC1(C)CCN(Cc2c(F)cc(N3CC(=O)NC4(CCN(c5cc(Cl)ncn5)CC4)C3)cc2F)CC1] | 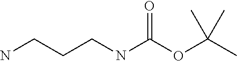 |
| | | 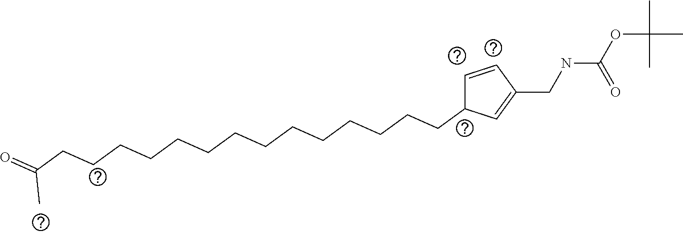 [SMILES: CC(=O)CCCCCCCCCCCCCCC1C=CC(CNC(=O)OC(C)(C)C)=C1] | 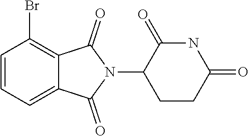 [SMILES: O=C1CCC(N2C(=O)c3cccc(Br)c3C2=O)C(=O)N1] |
| 168 | 4 | 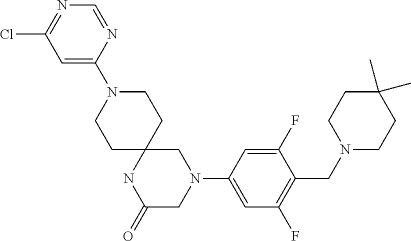 [SMILES: CC1(C)CCN(Cc2c(F)cc(N3CC(=O)NC4(CCN(c5cc(Cl)ncn5)CC4)C3)cc2F)CC1] | 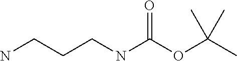 |
| | | 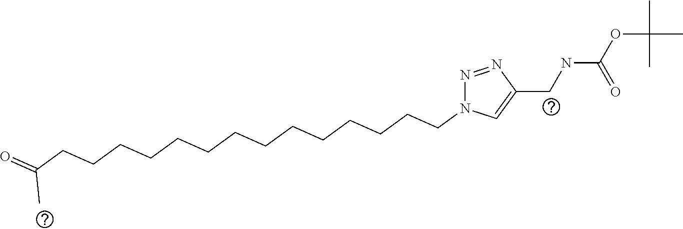 [SMILES: CC(=O)CCCCCCCCCCCCCCn1cc(CNC(=O)OC(C)(C)C)nn1] | 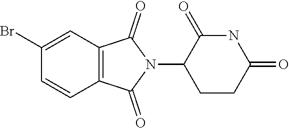 [SMILES: O=C1CCC(N2C(=O)c3ccc(Br)cc3C2=O)C(=O)N1] |
| 169 | 4 | 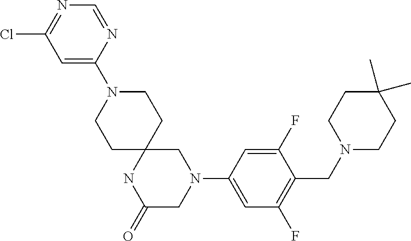 [SMILES: CC1(C)CCN(Cc2c(F)cc(N3CC(=O)NC4(CCN(c5cc(Cl)ncn5)CC4)C3)cc2F)CC1] | 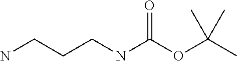 |
| | | 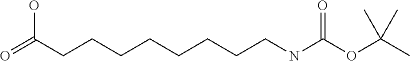 [SMILES: CC(C)(C)OC(=O)NCCCCCCCCC(=O)O] | 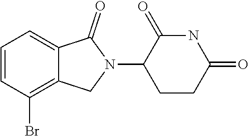 [SMILES: O=C1CCC(N2Cc3c(Br)cccc3C2=O)C(=O)N1] |
| 170 | 4 | 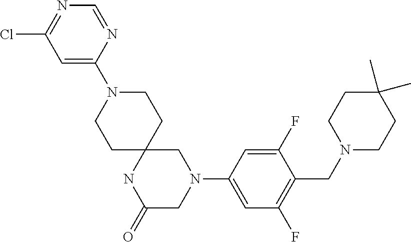 [SMILES: CC1(C)CCN(Cc2c(F)cc(N3CC(=O)NC4(CCN(c5cc(Cl)ncn5)CC4)C3)cc2F)CC1] | 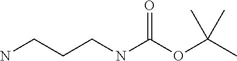 |
| | | 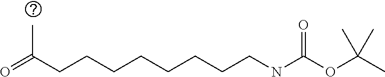 [SMILES: CC(=O)CCCCCCCCNC(=O)OC(C)(C)C] | 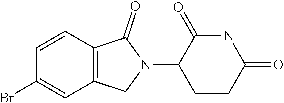 [SMILES: O=C1CCC(N2Cc3cc(Br)ccc3C2=O)C(=O)N1] |
| 171 | 4 | 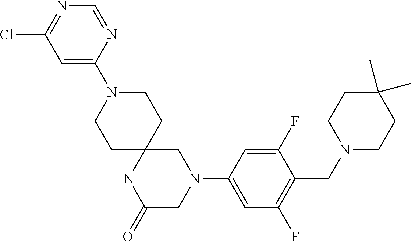 [SMILES: CC1(C)CCN(Cc2c(F)cc(N3CC(=O)NC4(CCN(c5cc(Cl)ncn5)CC4)C3)cc2F)CC1] | 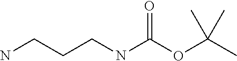 |
| | | 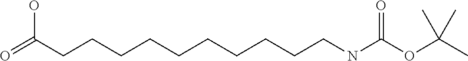 [SMILES: CC(C)(C)OC(=O)NCCCCCCCCCCC(=O)O] | 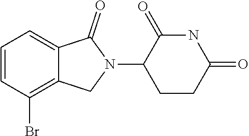 [SMILES: O=C1CCC(N2Cc3c(Br)cccc3C2=O)C(=O)N1] |
| 172 | 4 | 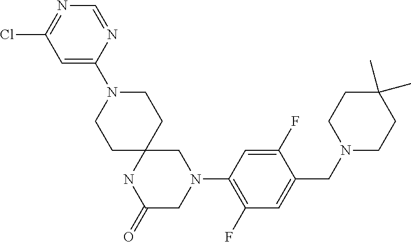 [SMILES: CC1(C)CCN(Cc2cc(F)c(N3CC(=O)NC4(CCN(c5cc(Cl)ncn5)CC4)C3)cc2F)CC1] | 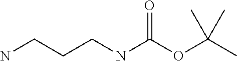 |
| | | 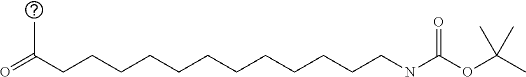 [SMILES: CC(=O)CCCCCCCCCCCCNC(=O)OC(C)(C)C] | 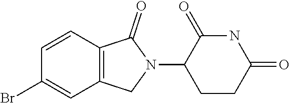 [SMILES: O=C1CCC(N2Cc3cc(Br)ccc3C2=O)C(=O)N1] |
| 173 | 4 | 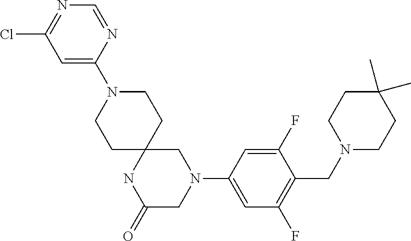 [SMILES: CC1(C)CCN(Cc2c(F)cc(N3CC(=O)NC4(CCN(c5cc(Cl)ncn5)CC4)C3)cc2F)CC1] | 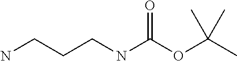 |
| | | 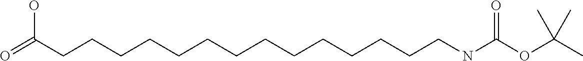 [SMILES: CC(C)(C)OC(=O)NCCCCCCCCCCCCCCC(=O)O] | 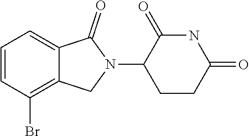 [SMILES: O=C1CCC(N2Cc3c(Br)cccc3C2=O)C(=O)N1] |
| 174 | 4 | 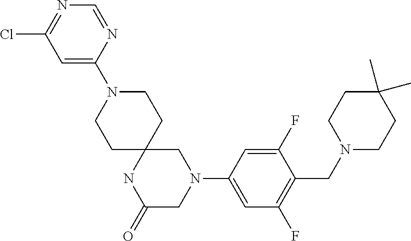 [SMILES: CC1(C)CCN(Cc2c(F)cc(N3CC(=O)NC4(CCN(c5cc(Cl)ncn5)CC4)C3)cc2F)CC1] | 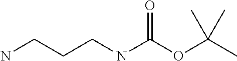 |
| | | 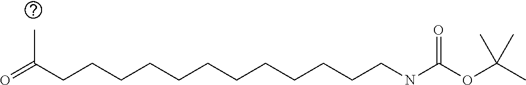 [SMILES: CC(=O)CCCCCCCCCCCCNC(=O)OC(C)(C)C] | 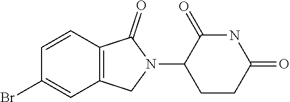 [SMILES: O=C1CCC(N2Cc3cc(Br)ccc3C2=O)C(=O)N1] |
| 175 | 4 | 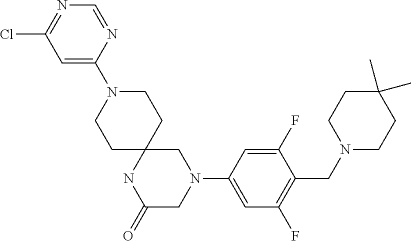 [SMILES: CC1(C)CCN(Cc2c(F)cc(N3CC(=O)NC4(CCN(c5cc(Cl)ncn5)CC4)C3)cc2F)CC1] | 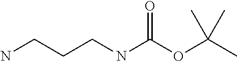 |
| | | 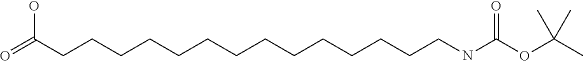 [SMILES: CC(C)(C)OC(=O)NCCCCCCCCCCCCCCC(=O)O] | 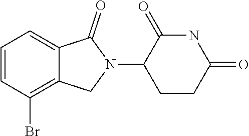 [SMILES: O=C1CCC(N2Cc3c(Br)cccc3C2=O)C(=O)N1] |
| 176 | 4 | 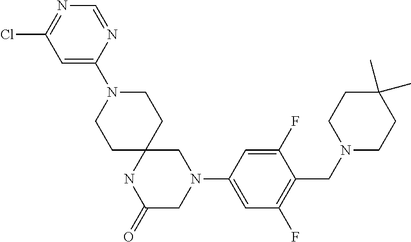 [SMILES: CC1(C)CCN(Cc2c(F)cc(N3CC(=O)NC4(CCN(c5cc(Cl)ncn5)CC4)C3)cc2F)CC1] | 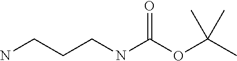 |
| | | 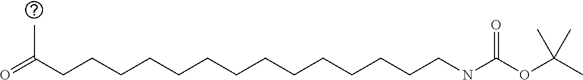 [SMILES: CC(=O)CCCCCCCCCCCCCCNC(=O)OC(C)(C)C] | 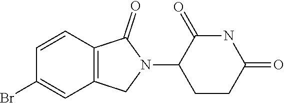 [SMILES: O=C1CCC(N2Cc3cc(Br)ccc3C2=O)C(=O)N1] |
| 177 | 4 | 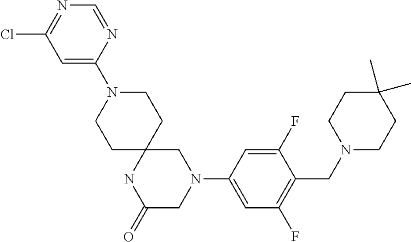 [SMILES: CC1(C)CCN(Cc2c(F)cc(N3CC(=O)NC4(CCN(c5cc(Cl)ncn5)CC4)C3)cc2F)CC1] | 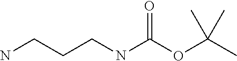 |
| | | 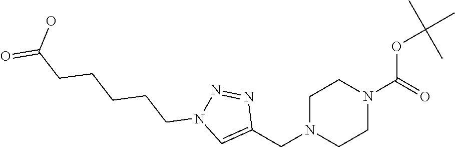 [SMILES: CC(C)(C)OC(=O)N1CCN(Cc2cn(CCCCCC(=O)O)nn2)CC1] | 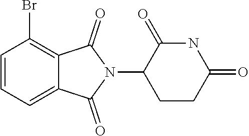 [SMILES: O=C1CCC(N2C(=O)c3cccc(Br)c3C2=O)C(=O)N1] |
| 178 | 4 | 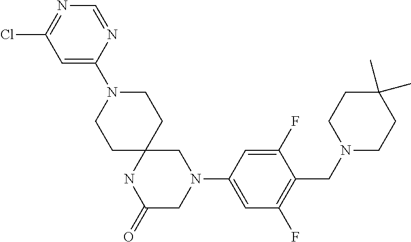 [SMILES: CC1(C)CCN(Cc2c(F)cc(N3CC(=O)NC4(CCN(c5cc(Cl)ncn5)CC4)C3)cc2F)CC1] | 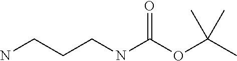 |
| | | 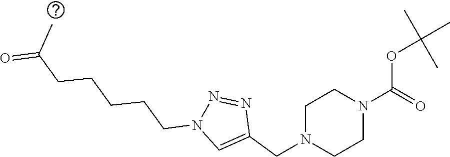 [SMILES: CC(=O)CCCCCn1cc(CN2CCN(C(=O)OC(C)(C)C)CC2)nn1] | 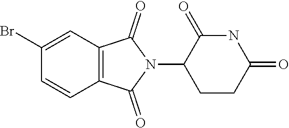 [SMILES: O=C1CCC(N2C(=O)c3ccc(Br)cc3C2=O)C(=O)N1] |
| 179 | 4 | 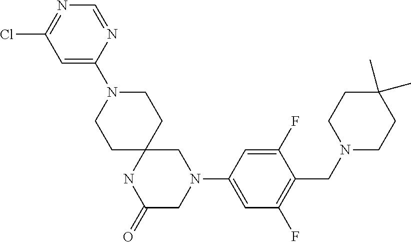 [SMILES: CC1(C)CCN(Cc2c(F)cc(N3CC(=O)NC4(CCN(c5cc(Cl)ncn5)CC4)C3)cc2F)CC1] | 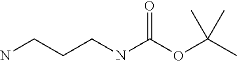 |
| | | 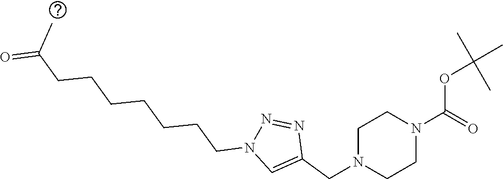 [SMILES: CC(=O)CCCCCCCn1cc(CN2CCN(C(=O)OC(C)(C)C)CC2)nn1] | 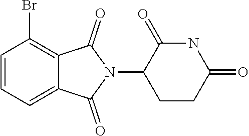 [SMILES: O=C1CCC(N2C(=O)c3cccc(Br)c3C2=O)C(=O)N1] |
| 180 | 4 | 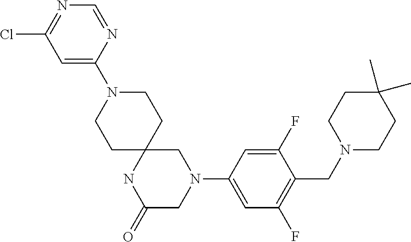 [SMILES: CC1(C)CCN(Cc2c(F)cc(N3CC(=O)NC4(CCN(c5cc(Cl)ncn5)CC4)C3)cc2F)CC1] | 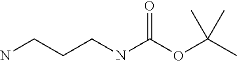 |
| | | 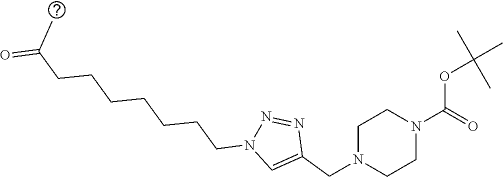 [SMILES: CC(=O)CCCCCCCn1cc(CN2CCN(C(=O)OC(C)(C)C)CC2)nn1] | 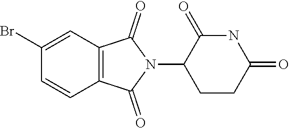 [SMILES: O=C1CCC(N2C(=O)c3ccc(Br)cc3C2=O)C(=O)N1] |
| 181 | 4 | 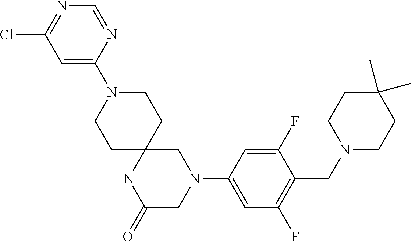 [SMILES: CC1(C)CCN(Cc2c(F)cc(N3CC(=O)NC4(CCN(c5cc(Cl)ncn5)CC4)C3)cc2F)CC1] | 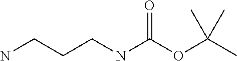 |
| | | 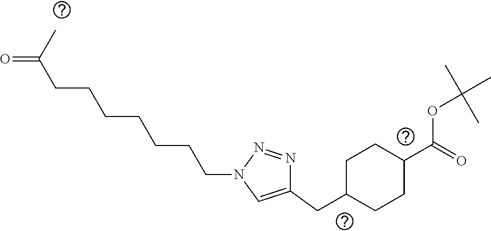 [SMILES: CC(=O)CCCCCCCn1cc(CC2CCC(C(=O)OC(C)(C)C)CC2)nn1] | 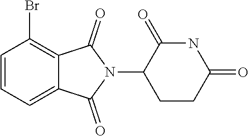 [SMILES: O=C1CCC(N2C(=O)c3cccc(Br)c3C2=O)C(=O)N1] |
| 182 | 4 | 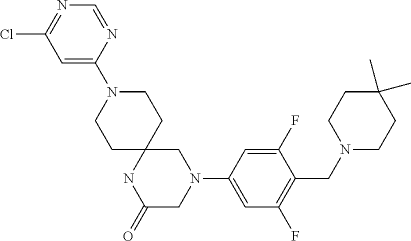 [SMILES: CC1(C)CCN(Cc2c(F)cc(N3CC(=O)NC4(CCN(c5cc(Cl)ncn5)CC4)C3)cc2F)CC1] | 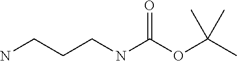 |
| | | 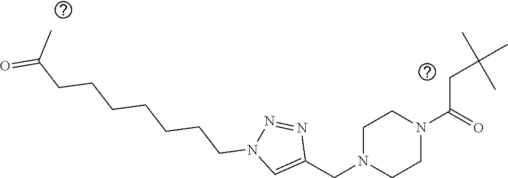 [SMILES: CC(=O)CCCCCCCn1cc(CN2CCN(C(=O)CC(C)(C)C)CC2)nn1] | 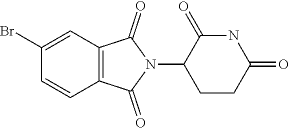 [SMILES: O=C1CCC(N2C(=O)c3ccc(Br)cc3C2=O)C(=O)N1] |
| 183 | 4 | 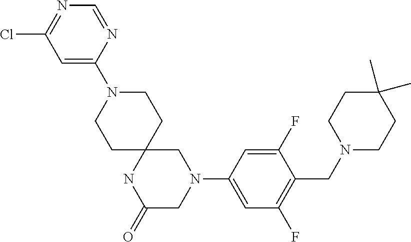 [SMILES: CC1(C)CCN(Cc2c(F)cc(N3CC(=O)NC4(CCN(c5cc(Cl)ncn5)CC4)C3)cc2F)CC1] | 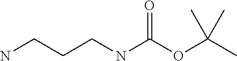 |
| | | 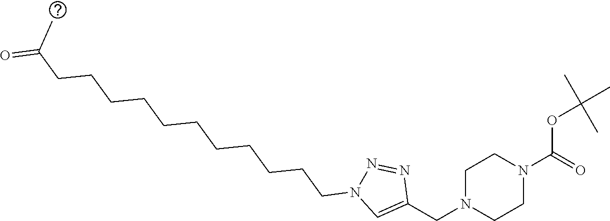 [SMILES: CC(=O)CCCCCCCCCCCn1cc(CN2CCN(C(=O)OC(C)(C)C)CC2)nn1] | 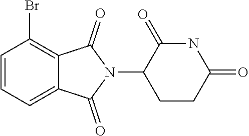 [SMILES: O=C1CCC(N2C(=O)c3cccc(Br)c3C2=O)C(=O)N1] |
| 184 | 4 | 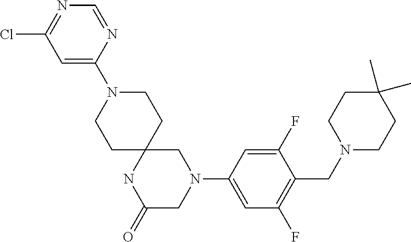 [SMILES: CC1(C)CCN(Cc2c(F)cc(N3CC(=O)NC4(CCN(c5cc(Cl)ncn5)CC4)C3)cc2F)CC1] | 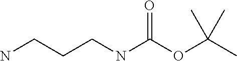 |
| | | 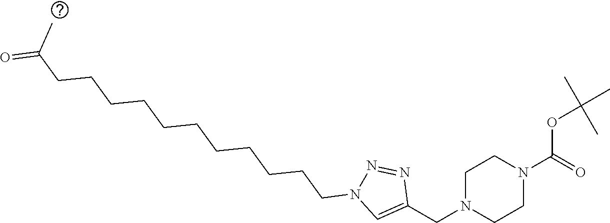 [SMILES: CC(=O)CCCCCCCCCCCn1cc(CN2CCN(C(=O)OC(C)(C)C)CC2)nn1] | 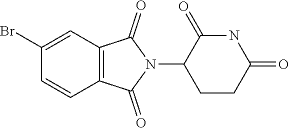 [SMILES: O=C1CCC(N2C(=O)c3ccc(Br)cc3C2=O)C(=O)N1] |
| 185 | 4 | 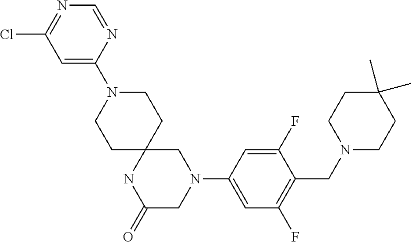 [SMILES: CC1(C)CCN(Cc2c(F)cc(N3CC(=O)NC4(CCN(c5cc(Cl)ncn5)CC4)C3)cc2F)CC1] | 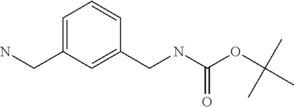 [SMILES: CC(C)(C)OC(=O)NCc1cccc(CN)c1] |
| | | 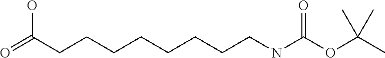 [SMILES: CC(C)(C)OC(=O)NCCCCCCCCC(=O)O] | 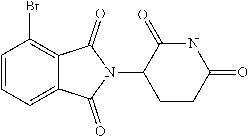 [SMILES: O=C1CCC(N2C(=O)c3cccc(Br)c3C2=O)C(=O)N1] |
| 186 | 4 | 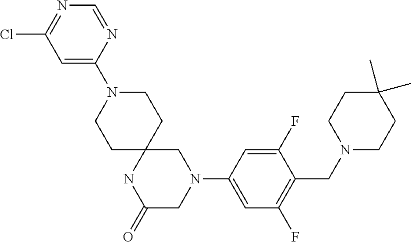 [SMILES: CC1(C)CCN(Cc2c(F)cc(N3CC(=O)NC4(CCN(c5cc(Cl)ncn5)CC4)C3)cc2F)CC1] | 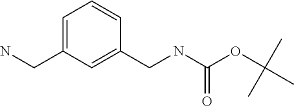 [SMILES: CC(C)(C)OC(=O)NCc1cccc(CN)c1] |
| | | 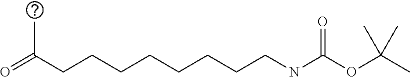 [SMILES: CC(=O)CCCCCCCCNC(=O)OC(C)(C)C] | 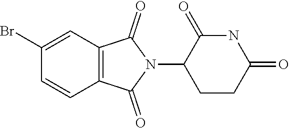 [SMILES: O=C1CCC(N2C(=O)c3ccc(Br)cc3C2=O)C(=O)N1] |
| 187 | 4 | 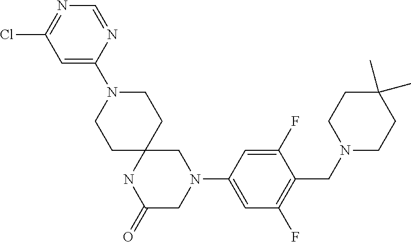 [SMILES: CC1(C)CCN(Cc2c(F)cc(N3CC(=O)NC4(CCN(c5cc(Cl)ncn5)CC4)C3)cc2F)CC1] | 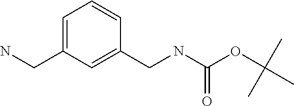 [SMILES: CC(C)(C)OC(=O)NCc1cccc(CN)c1] |
| | | 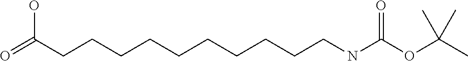 [SMILES: CC(C)(C)OC(=O)NCCCCCCCCCCC(=O)O] | 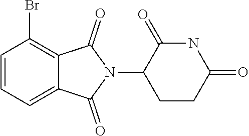 [SMILES: O=C1CCC(N2C(=O)c3cccc(Br)c3C2=O)C(=O)N1] |
| 188 | 4 | 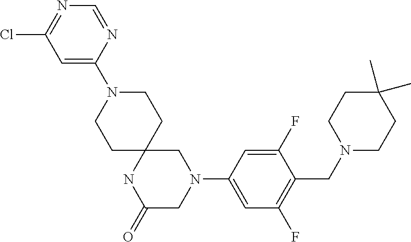 [SMILES: CC1(C)CCN(Cc2c(F)cc(N3CC(=O)NC4(CCN(c5cc(Cl)ncn5)CC4)C3)cc2F)CC1] | 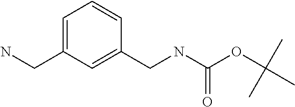 [SMILES: CC(C)(C)OC(=O)NCc1cccc(CN)c1] |
| | | 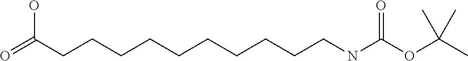 [SMILES: CC(C)(C)OC(=O)NCCCCCCCCCCC(=O)O] | 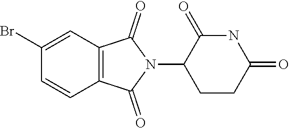 [SMILES: O=C1CCC(N2C(=O)c3ccc(Br)cc3C2=O)C(=O)N1] |
| 189 | 4 | 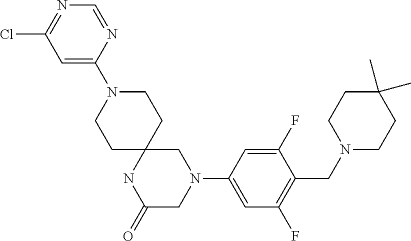 [SMILES: CC1(C)CCN(Cc2c(F)cc(N3CC(=O)NC4(CCN(c5cc(Cl)ncn5)CC4)C3)cc2F)CC1] | 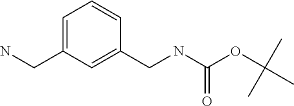 [SMILES: CC(C)(C)OC(=O)NCc1cccc(CN)c1] |
| | | 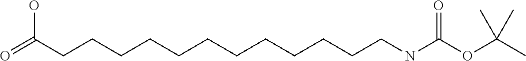 [SMILES: CC(C)(C)OC(=O)NCCCCCCCCCCCCC(=O)O] | 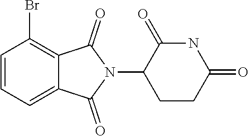 [SMILES: O=C1CCC(N2C(=O)c3cccc(Br)c3C2=O)C(=O)N1] |
| 190 | 4 | 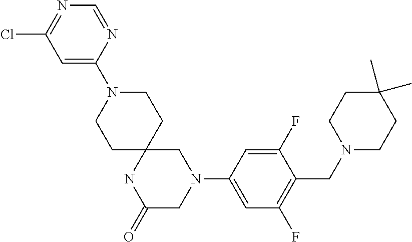 [SMILES: CC1(C)CCN(Cc2c(F)cc(N3CC(=O)NC4(CCN(c5cc(Cl)ncn5)CC4)C3)cc2F)CC1] | 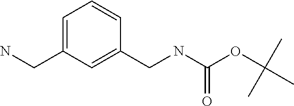 [SMILES: CC(C)(C)OC(=O)NCc1cccc(CN)c1] |
| | | 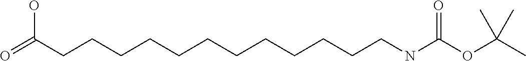 [SMILES: CC(C)(C)OC(=O)NCCCCCCCCCCCCC(=O)O] | 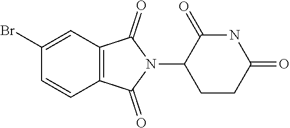 [SMILES: O=C1CCC(N2C(=O)c3ccc(Br)cc3C2=O)C(=O)N1] |
| 191 | 4 | 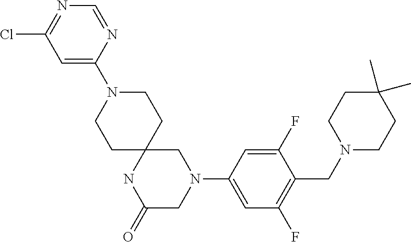 [SMILES: CC1(C)CCN(Cc2c(F)cc(N3CC(=O)NC4(CCN(c5cc(Cl)ncn5)CC4)C3)cc2F)CC1] | 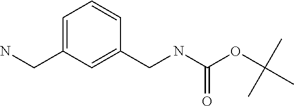 [SMILES: CC(C)(C)OC(=O)NCc1cccc(CN)c1] |
| | | 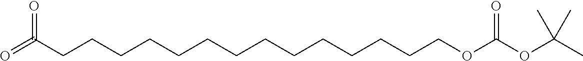 | 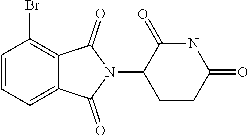 [SMILES: O=C1CCC(N2C(=O)c3cccc(Br)c3C2=O)C(=O)N1] |
| 192 | 4 | 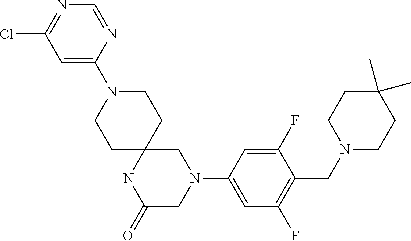 [SMILES: CC1(C)CCN(Cc2c(F)cc(N3CC(=O)NC4(CCN(c5cc(Cl)ncn5)CC4)C3)cc2F)CC1] | 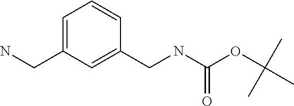 [SMILES: CC(C)(C)OC(=O)NCc1cccc(CN)c1] |
| | | 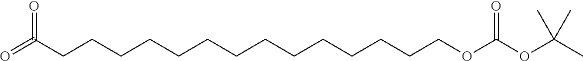 | 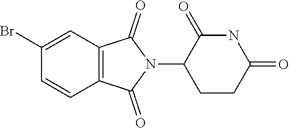 [SMILES: O=C1CCC(N2C(=O)c3ccc(Br)cc3C2=O)C(=O)N1] |
| 193 | 4 | 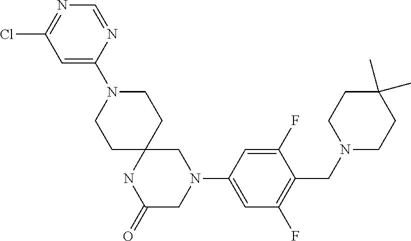 [SMILES: CC1(C)CCN(Cc2c(F)cc(N3CC(=O)NC4(CCN(c5cc(Cl)ncn5)CC4)C3)cc2F)CC1] | 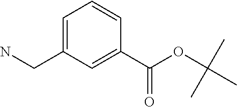 |
| | | 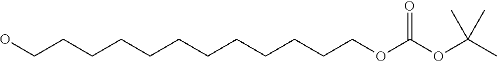 [SMILES: CC(C)(C)OC(=O)OCCCCCCCCCCCCO] | 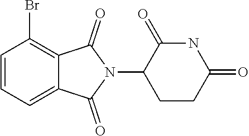 [SMILES: O=C1CCC(N2C(=O)c3cccc(Br)c3C2=O)C(=O)N1] |
| 194 | 4 | 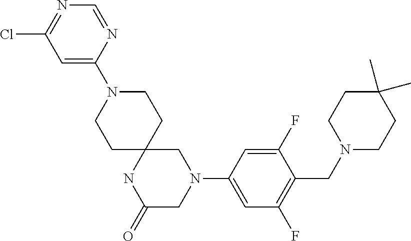 [SMILES: CC1(C)CCN(Cc2c(F)cc(N3CC(=O)NC4(CCN(c5cc(Cl)ncn5)CC4)C3)cc2F)CC1] | 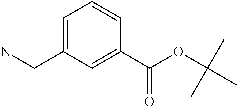 |
| | | 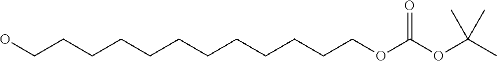 [SMILES: CC(C)(C)OC(=O)OCCCCCCCCCCCCO] | 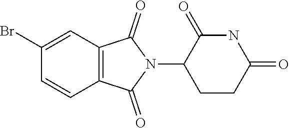 [SMILES: O=C1CCC(N2C(=O)c3ccc(Br)cc3C2=O)C(=O)N1] |
| 195 | 4 | 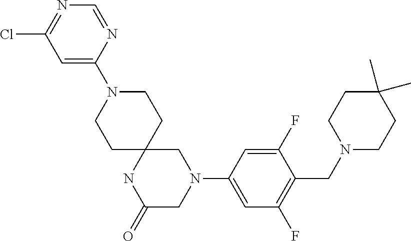 [SMILES: CC1(C)CCN(Cc2c(F)cc(N3CC(=O)NC4(CCN(c5cc(Cl)ncn5)CC4)C3)cc2F)CC1] | 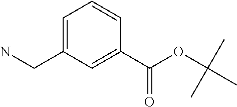 |
| | | 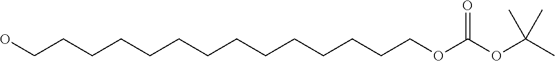 [SMILES: CC(C)(C)OC(=O)OCCCCCCCCCCCCCCO] | 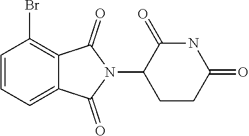 [SMILES: O=C1CCC(N2C(=O)c3cccc(Br)c3C2=O)C(=O)N1] |
| 196 | 4 | 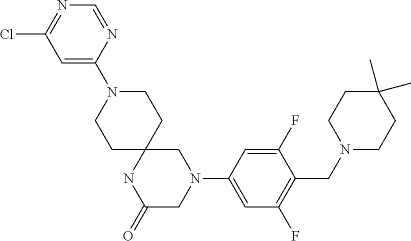 [SMILES: CC1(C)CCN(Cc2c(F)cc(N3CC(=O)NC4(CCN(c5cc(Cl)ncn5)CC4)C3)cc2F)CC1] | 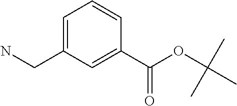 |
| | | 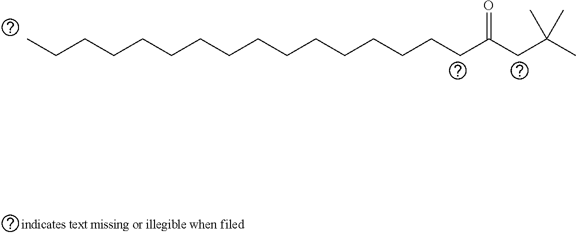 [SMILES: CCCCCCCCCCCCCCCCC(=O)CC(C)(C)C] | 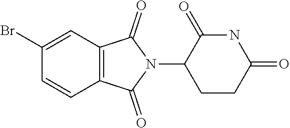 [SMILES: O=C1CCC(N2C(=O)c3ccc(Br)cc3C2=O)C(=O)N1] |
| 197 | 4 | 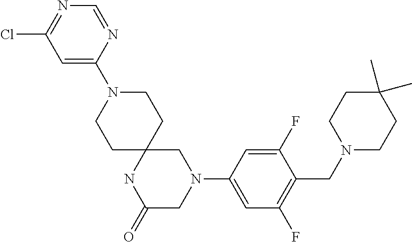 [SMILES: CC1(C)CCN(Cc2c(F)cc(N3CC(=O)NC4(CCN(c5cc(Cl)ncn5)CC4)C3)cc2F)CC1] | 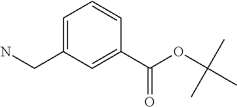 |
| | | 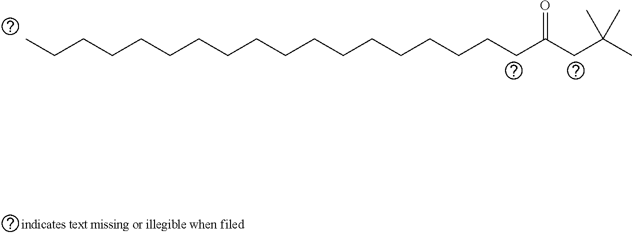 [SMILES: CCCCCCCCCCCCCCCCCCC(=O)CC(C)(C)C] | 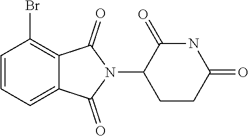 [SMILES: O=C1CCC(N2C(=O)c3cccc(Br)c3C2=O)C(=O)N1] |
| 198 | 4 | 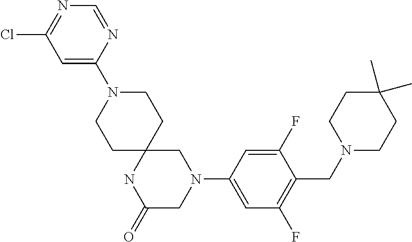 [SMILES: CC1(C)CCN(Cc2c(F)cc(N3CC(=O)NC4(CCN(c5cc(Cl)ncn5)CC4)C3)cc2F)CC1] | 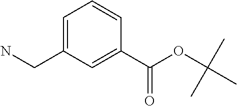 |
| | | 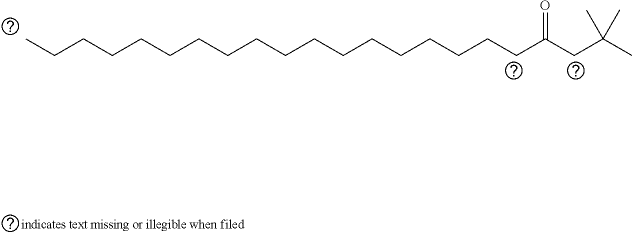 [SMILES: CCCCCCCCCCCCCCCCCCC(=O)CC(C)(C)C] | 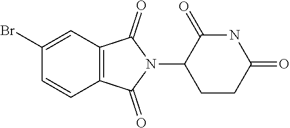 [SMILES: O=C1CCC(N2C(=O)c3ccc(Br)cc3C2=O)C(=O)N1] |
| 199 | 4 | 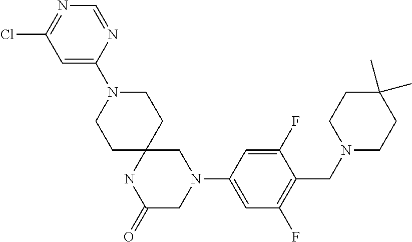 [SMILES: CC1(C)CCN(Cc2c(F)cc(N3CC(=O)NC4(CCN(c5cc(Cl)ncn5)CC4)C3)cc2F)CC1] | 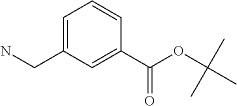 |
| | | 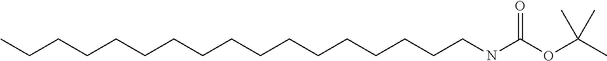 [SMILES: CCCCCCCCCCCCCCCCCNC(=O)OC(C)(C)C] | 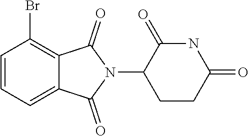 [SMILES: O=C1CCC(N2C(=O)c3cccc(Br)c3C2=O)C(=O)N1] |
| 200 | 4 | 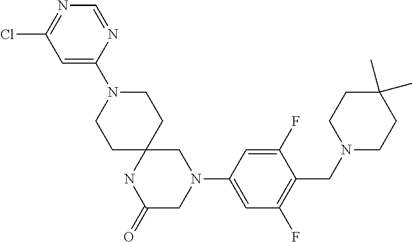 [SMILES: CC1(C)CCN(Cc2c(F)cc(N3CC(=O)NC4(CCN(c5cc(Cl)ncn5)CC4)C3)cc2F)CC1] | 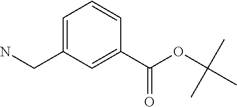 |
| | | 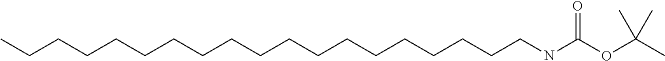 [SMILES: CCCCCCCCCCCCCCCCCCCNC(=O)OC(C)(C)C] | 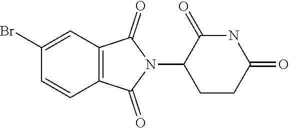 [SMILES: O=C1CCC(N2C(=O)c3ccc(Br)cc3C2=O)C(=O)N1] |
| 201 | 4 | 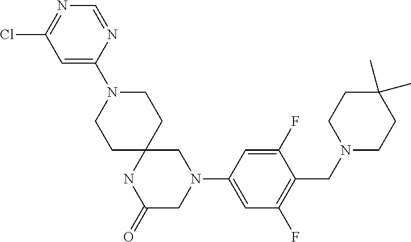 [SMILES: CC1(C)CCN(Cc2c(F)cc(N3CC(=O)NC4(CCN(c5cc(Cl)ncn5)CC4)C3)cc2F)CC1] | 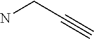 |
| | | 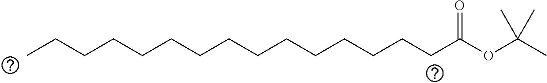 [SMILES: CCCCCCCCCCCCCCCC(=O)OC(C)(C)C] | 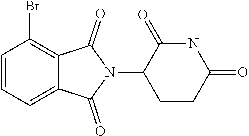 [SMILES: O=C1CCC(N2C(=O)c3cccc(Br)c3C2=O)C(=O)N1] |
| 202 | 4 | 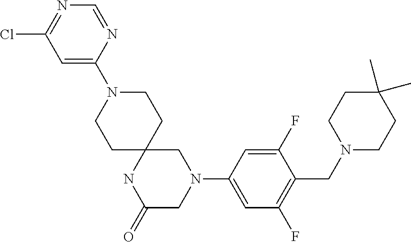 [SMILES: CC1(C)CCN(Cc2c(F)cc(N3CC(=O)NC4(CCN(c5cc(Cl)ncn5)CC4)C3)cc2F)CC1] | 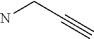 |
| | | 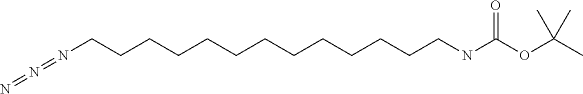 | 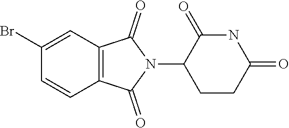 [SMILES: O=C1CCC(N2C(=O)c3ccc(Br)cc3C2=O)C(=O)N1] |
| 203 | 4 | 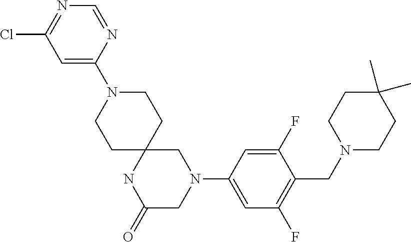 [SMILES: CC1(C)CCN(Cc2c(F)cc(N3CC(=O)NC4(CCN(c5cc(Cl)ncn5)CC4)C3)cc2F)CC1] | 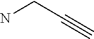 |
| | | 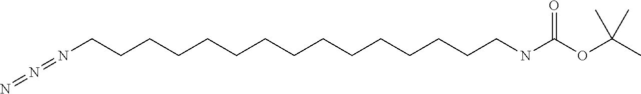 | 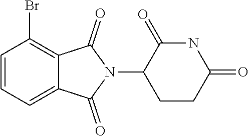 [SMILES: O=C1CCC(N2C(=O)c3cccc(Br)c3C2=O)C(=O)N1] |
| 204 | 4 | 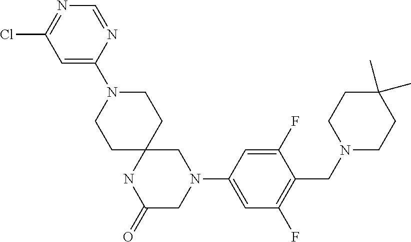 [SMILES: CC1(C)CCN(Cc2c(F)cc(N3CC(=O)NC4(CCN(c5cc(Cl)ncn5)CC4)C3)cc2F)CC1] | 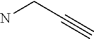 |
| | | 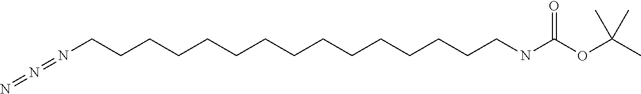 | 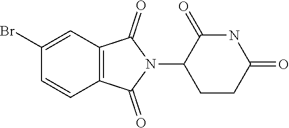 [SMILES: O=C1CCC(N2C(=O)c3ccc(Br)cc3C2=O)C(=O)N1] |
| 205 | 4 | 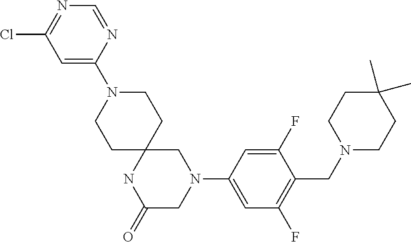 [SMILES: CC1(C)CCN(Cc2c(F)cc(N3CC(=O)NC4(CCN(c5cc(Cl)ncn5)CC4)C3)cc2F)CC1] | 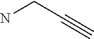 |
| | | 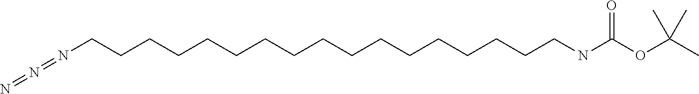 | 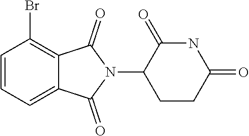 [SMILES: O=C1CCC(N2C(=O)c3cccc(Br)c3C2=O)C(=O)N1] |
| 206 | 4 | 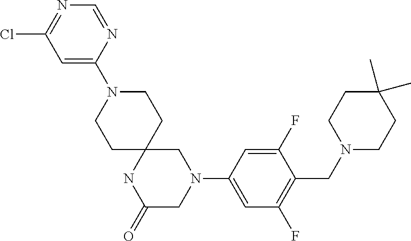 [SMILES: CC1(C)CCN(Cc2c(F)cc(N3CC(=O)NC4(CCN(c5cc(Cl)ncn5)CC4)C3)cc2F)CC1] | 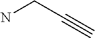 |
| | | 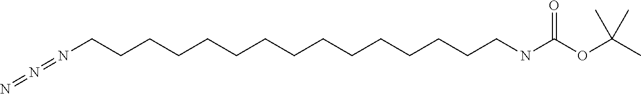 | 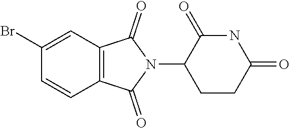 [SMILES: O=C1CCC(N2C(=O)c3ccc(Br)cc3C2=O)C(=O)N1] |
| 207 | 4 | 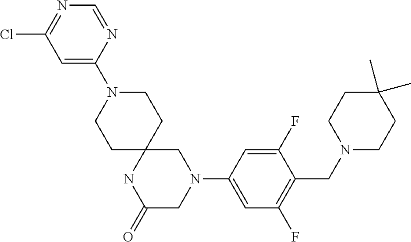 [SMILES: CC1(C)CCN(Cc2c(F)cc(N3CC(=O)NC4(CCN(c5cc(Cl)ncn5)CC4)C3)cc2F)CC1] | 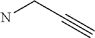 |
| | | 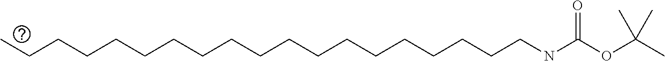 [SMILES: CCCCCCCCCCCCCCCCCCCNC(=O)OC(C)(C)C] | 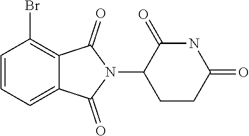 [SMILES: O=C1CCC(N2C(=O)c3cccc(Br)c3C2=O)C(=O)N1] |
| 208 | 4 | 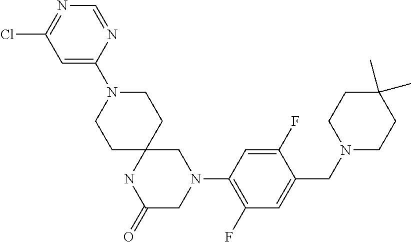 [SMILES: CC1(C)CCN(Cc2cc(F)c(N3CC(=O)NC4(CCN(c5cc(Cl)ncn5)CC4)C3)cc2F)CC1] | 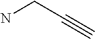 |
| | | 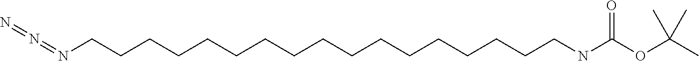 | 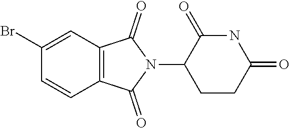 [SMILES: O=C1CCC(N2C(=O)c3ccc(Br)cc3C2=O)C(=O)N1] |
| 210 | 5 | 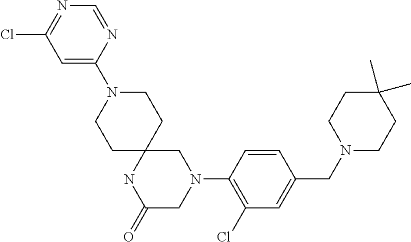 [SMILES: CC1(C)CCN(Cc2ccc(N3CC(=O)NC4(CCN(c5cc(Cl)ncn5)CC4)C3)c(Cl)c2)CC1] | 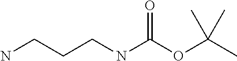 |
| | | 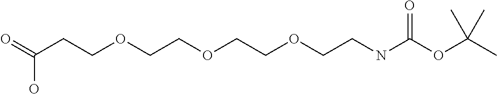 [SMILES: CC(C)(C)OC(=O)NCCOCCOCCOCCC(=O)O] | 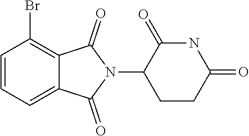 [SMILES: O=C1CCC(N2C(=O)c3cccc(Br)c3C2=O)C(=O)N1] |
| 211 | 5 | 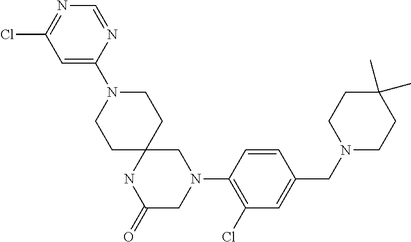 [SMILES: CC1(C)CCN(Cc2ccc(N3CC(=O)NC4(CCN(c5cc(Cl)ncn5)CC4)C3)c(Cl)c2)CC1] | 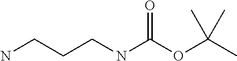 |
| | | 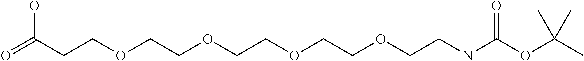 [SMILES: CC(C)(C)OC(=O)NCCOCCOCCOCCOCCC(=O)O] | 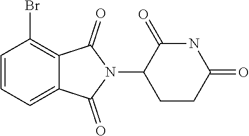 [SMILES: O=C1CCC(N2C(=O)c3cccc(Br)c3C2=O)C(=O)N1] |
| 212 | 5 | 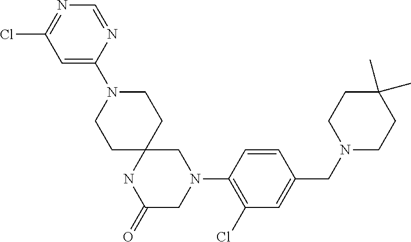 [SMILES: CC1(C)CCN(Cc2ccc(N3CC(=O)NC4(CCN(c5cc(Cl)ncn5)CC4)C3)c(Cl)c2)CC1] | 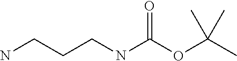 |
| | | 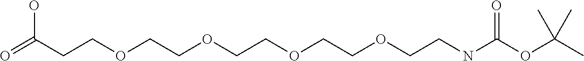 [SMILES: CC(C)(C)OC(=O)NCCOCCOCCOCCOCCC(=O)O] | 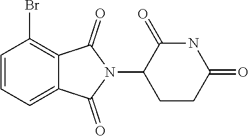 [SMILES: O=C1CCC(N2C(=O)c3cccc(Br)c3C2=O)C(=O)N1] |
| 212 | 5 | 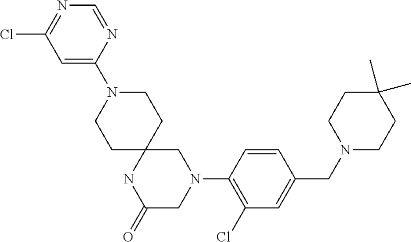 [SMILES: CC1(C)CCN(Cc2ccc(N3CC(=O)NC4(CCN(c5cc(Cl)ncn5)CC4)C3)c(Cl)c2)CC1] | 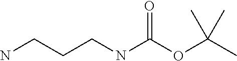 |
| | | 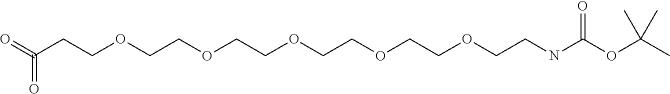 | 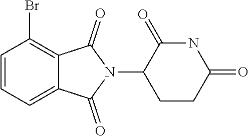 [SMILES: O=C1CCC(N2C(=O)c3cccc(Br)c3C2=O)C(=O)N1] |
| 213 | 5 | 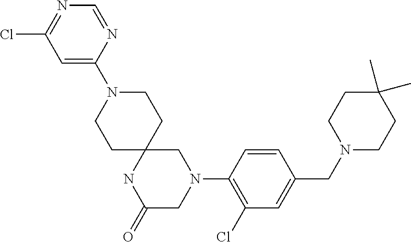 [SMILES: CC1(C)CCN(Cc2ccc(N3CC(=O)NC4(CCN(c5cc(Cl)ncn5)CC4)C3)c(Cl)c2)CC1] | 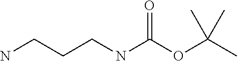 |
| | | 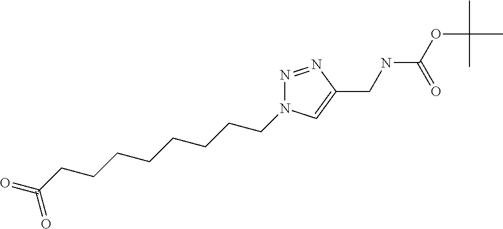 | 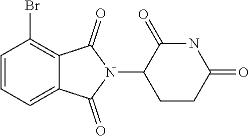 [SMILES: O=C1CCC(N2C(=O)c3cccc(Br)c3C2=O)C(=O)N1] |
| 214 | 5 | 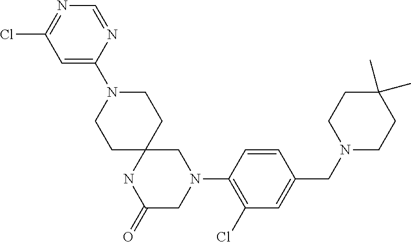 [SMILES: CC1(C)CCN(Cc2ccc(N3CC(=O)NC4(CCN(c5cc(Cl)ncn5)CC4)C3)c(Cl)c2)CC1] | 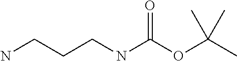 |
| | | 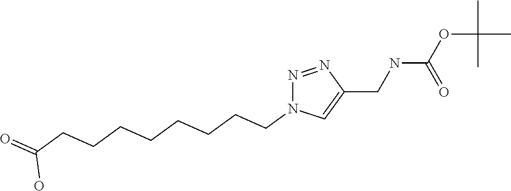 [SMILES: CC(C)(C)OC(=O)NCc1cn(CCCCCCCCC(=O)O)nn1] | 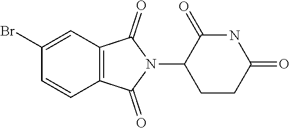 [SMILES: O=C1CCC(N2C(=O)c3ccc(Br)cc3C2=O)C(=O)N1] |
| 215 | 5 | 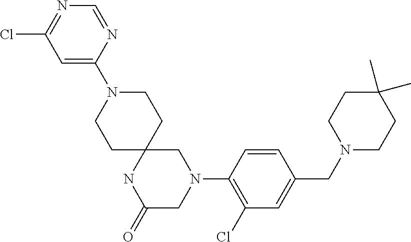 [SMILES: CC1(C)CCN(Cc2ccc(N3CC(=O)NC4(CCN(c5cc(Cl)ncn5)CC4)C3)c(Cl)c2)CC1] | 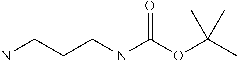 |
| | | 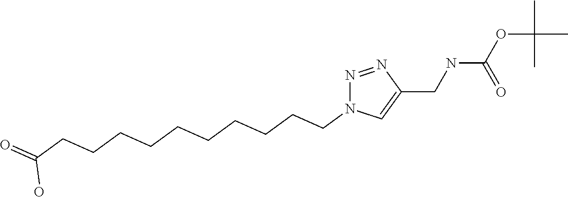 [SMILES: CC(C)(C)OC(=O)NCc1cn(CCCCCCCCCCC(=O)O)nn1] | 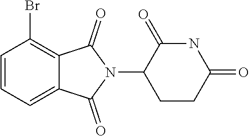 [SMILES: O=C1CCC(N2C(=O)c3cccc(Br)c3C2=O)C(=O)N1] |
| 216 | 5 | 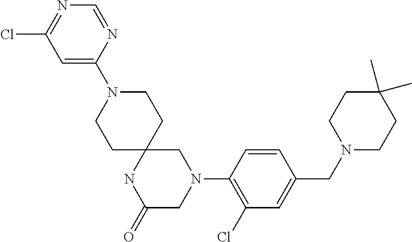 [SMILES: CC1(C)CCN(Cc2ccc(N3CC(=O)NC4(CCN(c5cc(Cl)ncn5)CC4)C3)c(Cl)c2)CC1] | 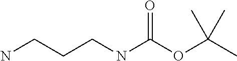 |
| | | 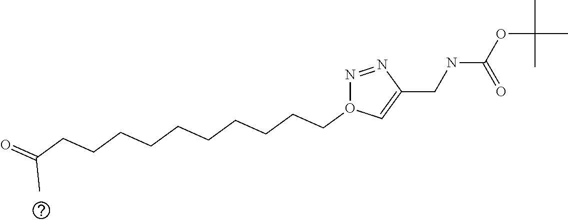 | 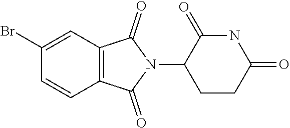 [SMILES: O=C1CCC(N2C(=O)c3ccc(Br)cc3C2=O)C(=O)N1] |
| 217 | 5 | 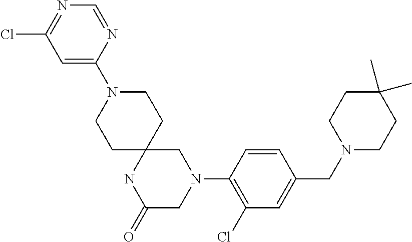 [SMILES: CC1(C)CCN(Cc2ccc(N3CC(=O)NC4(CCN(c5cc(Cl)ncn5)CC4)C3)c(Cl)c2)CC1] | 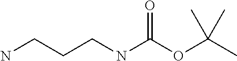 |
| | | 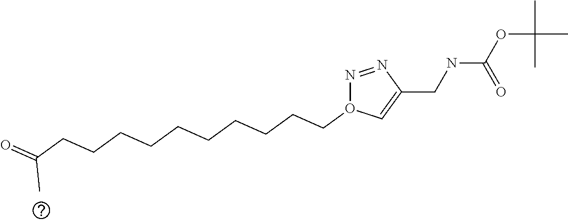 | 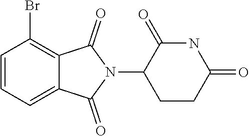 [SMILES: O=C1CCC(N2C(=O)c3cccc(Br)c3C2=O)C(=O)N1] |
| 218 | 5 | 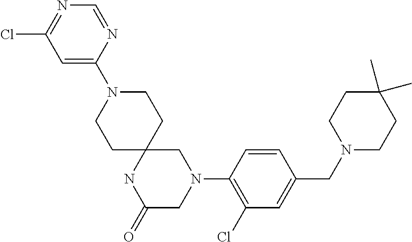 [SMILES: CC1(C)CCN(Cc2ccc(N3CC(=O)NC4(CCN(c5cc(Cl)ncn5)CC4)C3)c(Cl)c2)CC1] | 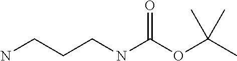 |
| | | 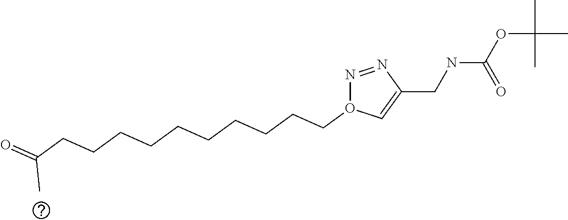 | 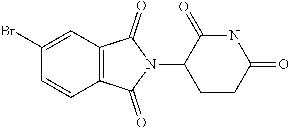 [SMILES: O=C1CCC(N2C(=O)c3ccc(Br)cc3C2=O)C(=O)N1] |
| 219 | 5 | 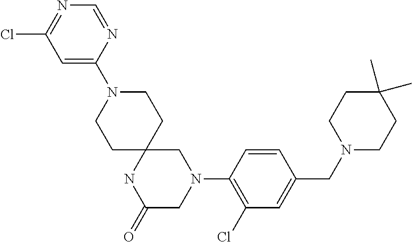 [SMILES: CC1(C)CCN(Cc2ccc(N3CC(=O)NC4(CCN(c5cc(Cl)ncn5)CC4)C3)c(Cl)c2)CC1] | 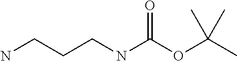 |
| | | 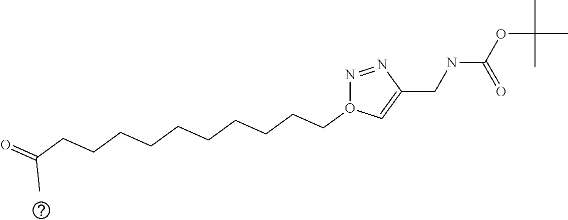 | 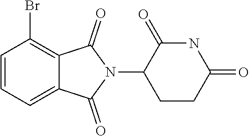 [SMILES: O=C1CCC(N2C(=O)c3cccc(Br)c3C2=O)C(=O)N1] |
| 220 | 5 | 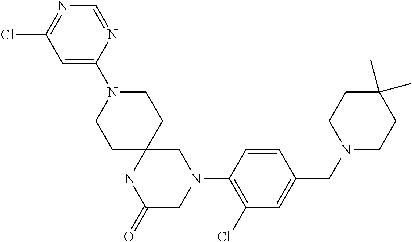 [SMILES: CC1(C)CCN(Cc2ccc(N3CC(=O)NC4(CCN(c5cc(Cl)ncn5)CC4)C3)c(Cl)c2)CC1] | 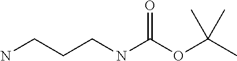 |
| | | 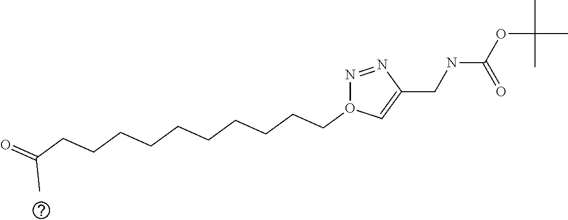 | 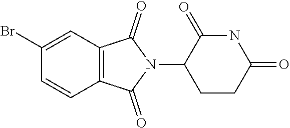 [SMILES: O=C1CCC(N2C(=O)c3ccc(Br)cc3C2=O)C(=O)N1] |
| 220 | 5 | 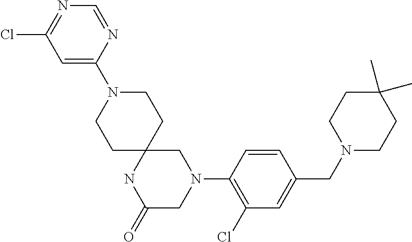 [SMILES: CC1(C)CCN(Cc2ccc(N3CC(=O)NC4(CCN(c5cc(Cl)ncn5)CC4)C3)c(Cl)c2)CC1] | 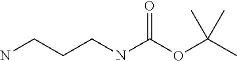 |
| | | 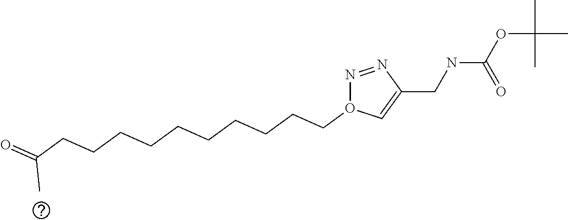 | 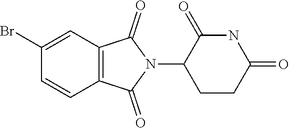 [SMILES: O=C1CCC(N2C(=O)c3ccc(Br)cc3C2=O)C(=O)N1] |
| 221 | 5 | 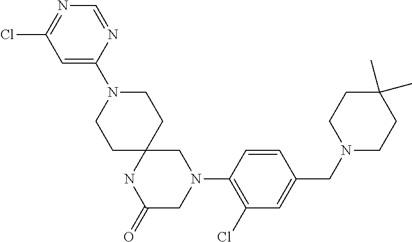 [SMILES: CC1(C)CCN(Cc2ccc(N3CC(=O)NC4(CCN(c5cc(Cl)ncn5)CC4)C3)c(Cl)c2)CC1] | 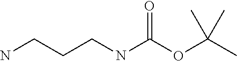 |
| | | 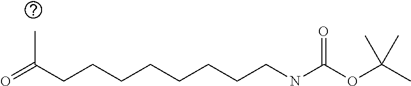 [SMILES: CC(=O)CCCCCCCCNC(=O)OC(C)(C)C] | 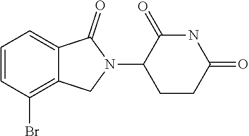 [SMILES: O=C1CCC(N2Cc3c(Br)cccc3C2=O)C(=O)N1] |
| 222 | 5 | 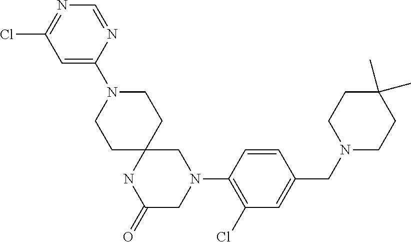 [SMILES: CC1(C)CCN(Cc2ccc(N3CC(=O)NC4(CCN(c5cc(Cl)ncn5)CC4)C3)c(Cl)c2)CC1] | 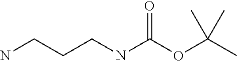 |
| | | 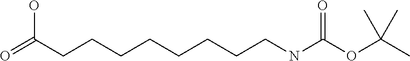 [SMILES: CC(C)(C)OC(=O)NCCCCCCCCC(=O)O] | 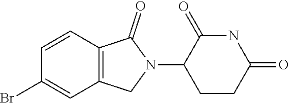 [SMILES: O=C1CCC(N2Cc3cc(Br)ccc3C2=O)C(=O)N1] |
| 223 | 5 | 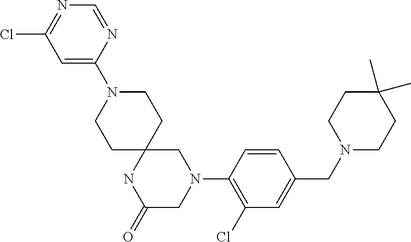 [SMILES: CC1(C)CCN(Cc2ccc(N3CC(=O)NC4(CCN(c5cc(Cl)ncn5)CC4)C3)c(Cl)c2)CC1] | 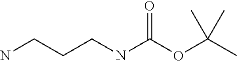 |
| | | 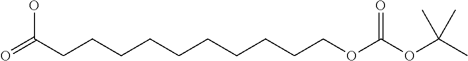 [SMILES: CC(C)(C)OC(=O)OCCCCCCCCCCC(=O)O] | 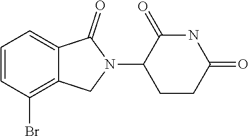 [SMILES: O=C1CCC(N2Cc3c(Br)cccc3C2=O)C(=O)N1] |
| 224 | 5 | 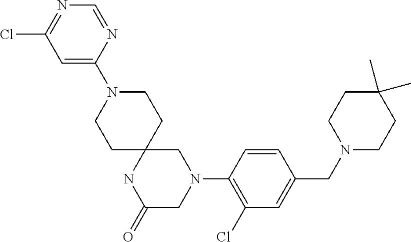 [SMILES: CC1(C)CCN(Cc2ccc(N3CC(=O)NC4(CCN(c5cc(Cl)ncn5)CC4)C3)c(Cl)c2)CC1] | 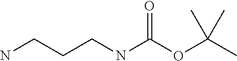 |
| | | 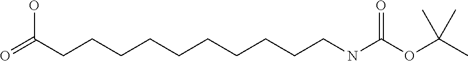 [SMILES: CC(C)(C)OC(=O)NCCCCCCCCCCC(=O)O] | 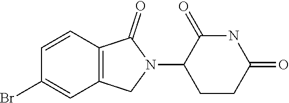 [SMILES: O=C1CCC(N2Cc3cc(Br)ccc3C2=O)C(=O)N1] |
| 225 | 5 | 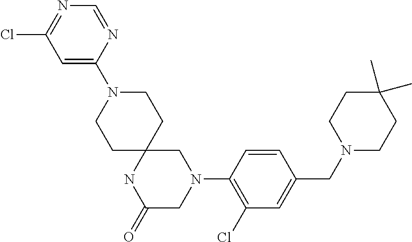 [SMILES: CC1(C)CCN(Cc2ccc(N3CC(=O)NC4(CCN(c5cc(Cl)ncn5)CC4)C3)c(Cl)c2)CC1] | 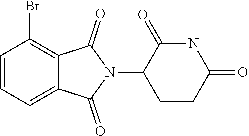 [SMILES: O=C1CCC(N2C(=O)c3cccc(Br)c3C2=O)C(=O)N1] |
| | | 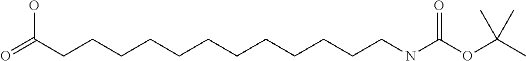 [SMILES: CC(C)(C)OC(=O)NCCCCCCCCCCCCC(=O)O] | 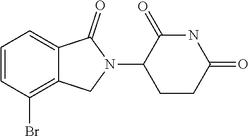 [SMILES: O=C1CCC(N2Cc3c(Br)cccc3C2=O)C(=O)N1] |
| 226 | 5 | 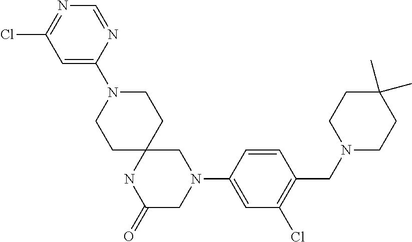 [SMILES: CC1(C)CCN(Cc2ccc(N3CC(=O)NC4(CCN(c5cc(Cl)ncn5)CC4)C3)cc2Cl)CC1] | 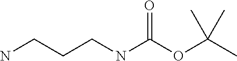 |
| | | 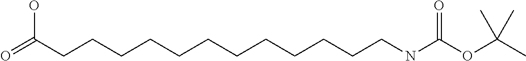 [SMILES: CC(C)(C)OC(=O)NCCCCCCCCCCCCC(=O)O] | 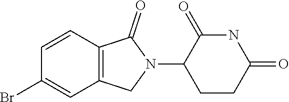 [SMILES: O=C1CCC(N2Cc3cc(Br)ccc3C2=O)C(=O)N1] |
| 227 | 5 | 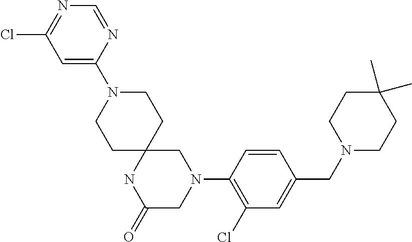 [SMILES: CC1(C)CCN(Cc2ccc(N3CC(=O)NC4(CCN(c5cc(Cl)ncn5)CC4)C3)c(Cl)c2)CC1] | 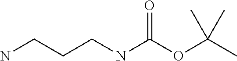 |
| | | 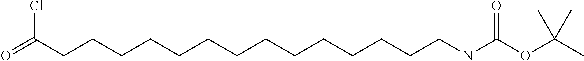 [SMILES: CC(C)(C)OC(=O)NCCCCCCCCCCCCCCC(=O)Cl] | 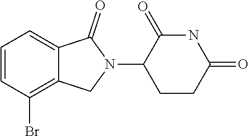 [SMILES: O=C1CCC(N2Cc3c(Br)cccc3C2=O)C(=O)N1] |
| 228 | 5 | 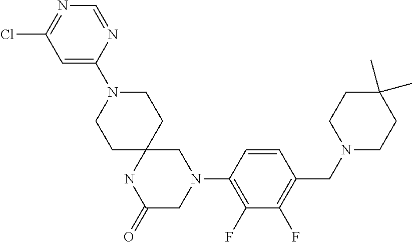 [SMILES: CC1(C)CCN(Cc2ccc(N3CC(=O)NC4(CCN(c5cc(Cl)ncn5)CC4)C3)c(F)c2F)CC1] | 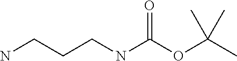 |
| | | 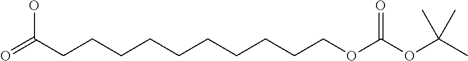 [SMILES: CC(C)(C)OC(=O)OCCCCCCCCCCC(=O)O] | 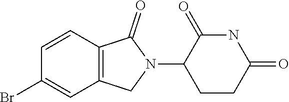 [SMILES: O=C1CCC(N2Cc3cc(Br)ccc3C2=O)C(=O)N1] |
| 229 | 5 | 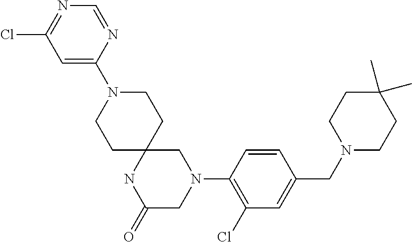 [SMILES: CC1(C)CCN(Cc2ccc(N3CC(=O)NC4(CCN(c5cc(Cl)ncn5)CC4)C3)c(Cl)c2)CC1] | 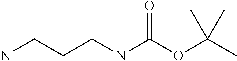 |
| | | 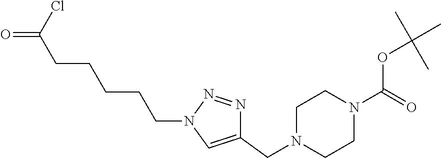 [SMILES: CC(C)(C)OC(=O)N1CCN(Cc2cn(CCCCCC(=O)Cl)nn2)CC1] | 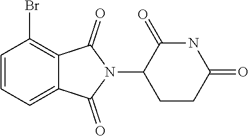 [SMILES: O=C1CCC(N2C(=O)c3cccc(Br)c3C2=O)C(=O)N1] |
| 230 | 5 | 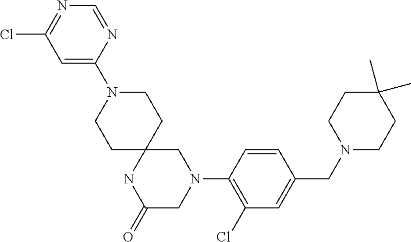 [SMILES: CC1(C)CCN(Cc2ccc(N3CC(=O)NC4(CCN(c5cc(Cl)ncn5)CC4)C3)c(Cl)c2)CC1] | 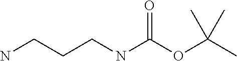 |
| | | 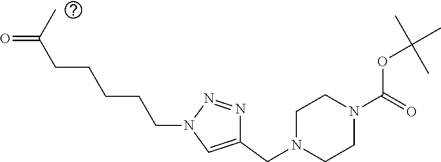 [SMILES: CC(=O)CCCCCn1cc(CN2CCN(C(=O)OC(C)(C)C)CC2)nn1] | 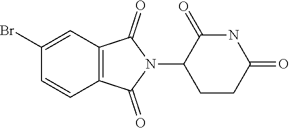 [SMILES: O=C1CCC(N2C(=O)c3ccc(Br)cc3C2=O)C(=O)N1] |
| 231 | 5 | 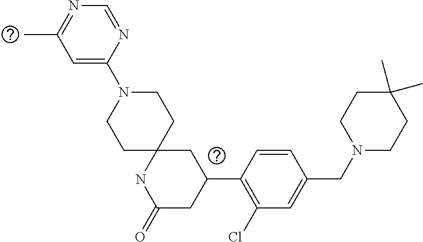 | 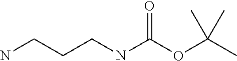 |
| | | 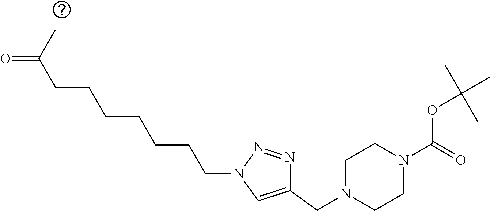 [SMILES: CC(=O)CCCCCCCn1cc(CN2CCN(C(=O)OC(C)(C)C)CC2)nn1] | 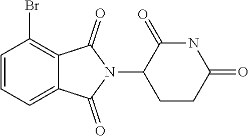 [SMILES: O=C1CCC(N2C(=O)c3cccc(Br)c3C2=O)C(=O)N1] |
| 232 | 5 | 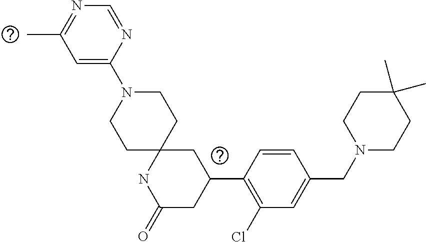 | 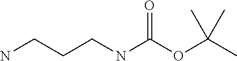 |
| | | 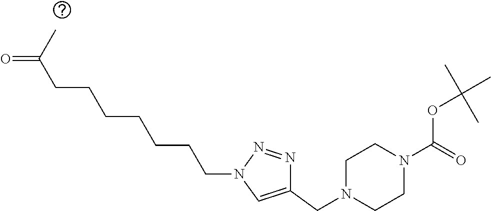 [SMILES: CC(=O)CCCCCCCn1cc(CN2CCN(C(=O)OC(C)(C)C)CC2)nn1] | 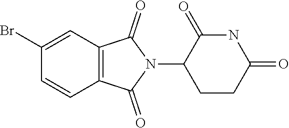 [SMILES: O=C1CCC(N2C(=O)c3ccc(Br)cc3C2=O)C(=O)N1] |
| 233 | 5 | 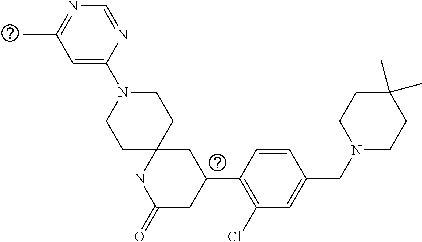 | 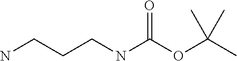 |
| | | 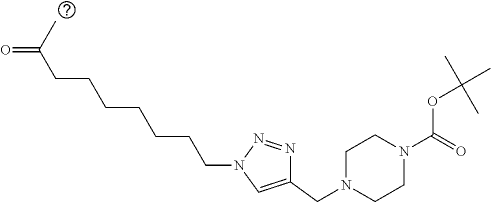 [SMILES: CC(=O)CCCCCCCn1cc(CN2CCN(C(=O)OC(C)(C)C)CC2)nn1] | 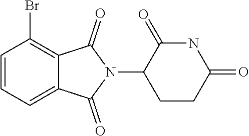 [SMILES: O=C1CCC(N2C(=O)c3cccc(Br)c3C2=O)C(=O)N1] |
| 234 | 5 | 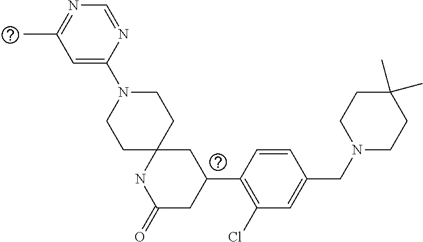 | 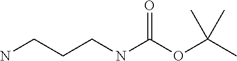 |
| | | 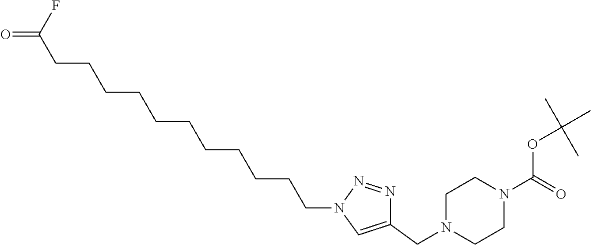 [SMILES: CC(C)(C)OC(=O)N1CCN(Cc2cn(CCCCCCCCCCCC(=O)F)nn2)CC1] | 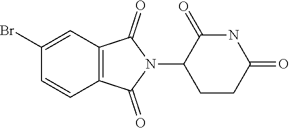 [SMILES: O=C1CCC(N2C(=O)c3ccc(Br)cc3C2=O)C(=O)N1] |
| 235 | 5 | 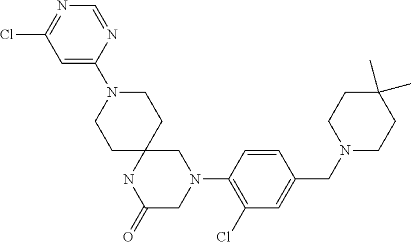 [SMILES: CC1(C)CCN(Cc2ccc(N3CC(=O)NC4(CCN(c5cc(Cl)ncn5)CC4)C3)c(Cl)c2)CC1] | 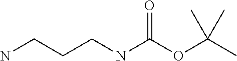 |
| | | 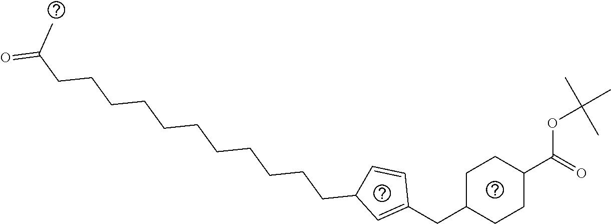 [SMILES: CC(=O)CCCCCCCCCCCC1C=CC(CC2CCC(C(=O)OC(C)(C)C)CC2)=C1] | 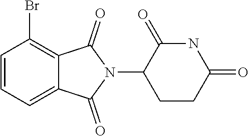 [SMILES: O=C1CCC(N2C(=O)c3cccc(Br)c3C2=O)C(=O)N1] |
| 236 | 5 | 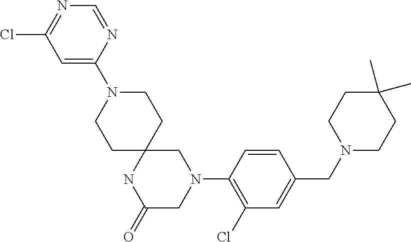 [SMILES: CC1(C)CCN(Cc2ccc(N3CC(=O)NC4(CCN(c5cc(Cl)ncn5)CC4)C3)c(Cl)c2)CC1] | 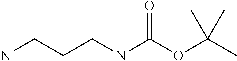 |
| | | 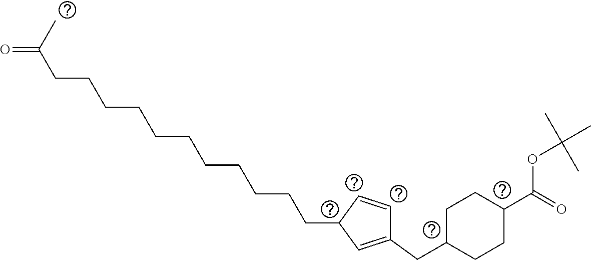 [SMILES: CC(=O)CCCCCCCCCCCC1C=CC(CC2CCC(C(=O)OC(C)(C)C)CC2)=C1] | 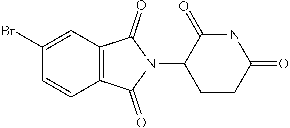 [SMILES: O=C1CCC(N2C(=O)c3ccc(Br)cc3C2=O)C(=O)N1] |
| 237 | 5 | 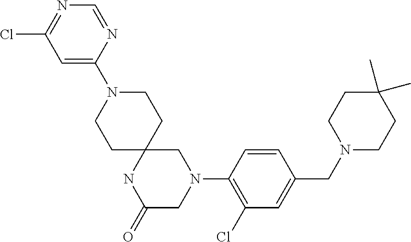 [SMILES: CC1(C)CCN(Cc2ccc(N3CC(=O)NC4(CCN(c5cc(Cl)ncn5)CC4)C3)c(Cl)c2)CC1] | 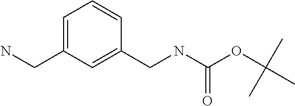 [SMILES: CC(C)(C)OC(=O)NCc1cccc(CN)c1] |
| | | 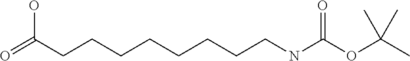 [SMILES: CC(C)(C)OC(=O)NCCCCCCCCC(=O)O] | 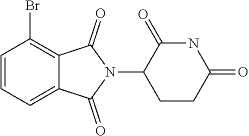 [SMILES: O=C1CCC(N2C(=O)c3cccc(Br)c3C2=O)C(=O)N1] |
| 238 | 5 | 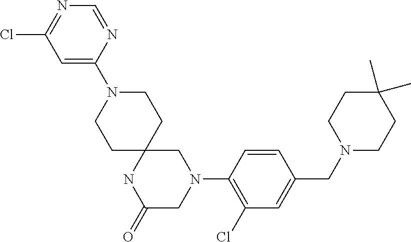 [SMILES: CC1(C)CCN(Cc2ccc(N3CC(=O)NC4(CCN(c5cc(Cl)ncn5)CC4)C3)c(Cl)c2)CC1] | 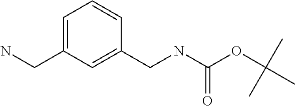 [SMILES: CC(C)(C)OC(=O)NCc1cccc(CN)c1] |
| | | 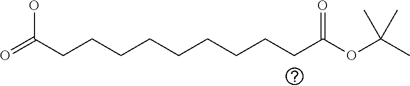 [SMILES: CC(C)(C)OC(=O)CCCCCCCCCC(=O)O] | 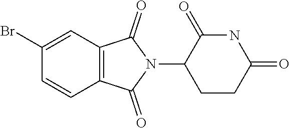 [SMILES: O=C1CCC(N2C(=O)c3ccc(Br)cc3C2=O)C(=O)N1] |
| 239 | 5 | 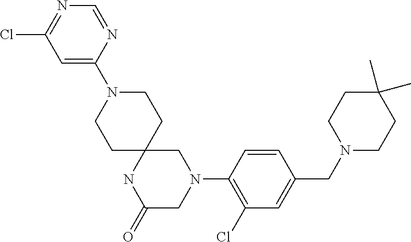 [SMILES: CC1(C)CCN(Cc2ccc(N3CC(=O)NC4(CCN(c5cc(Cl)ncn5)CC4)C3)c(Cl)c2)CC1] | 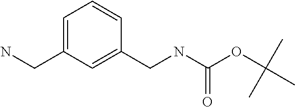 [SMILES: CC(C)(C)OC(=O)NCc1cccc(CN)c1] |
| | | 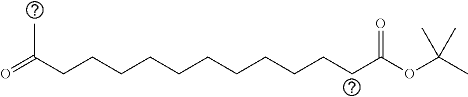 [SMILES: CC(=O)CCCCCCCCCCCC(=O)OC(C)(C)C] | 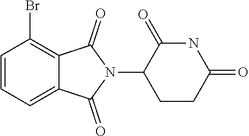 [SMILES: O=C1CCC(N2C(=O)c3cccc(Br)c3C2=O)C(=O)N1] |
| 240 | 5 | 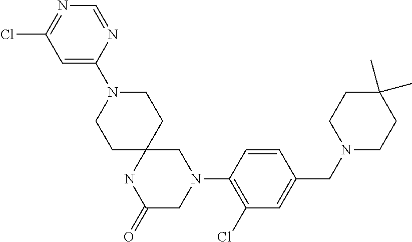 [SMILES: CC1(C)CCN(Cc2ccc(N3CC(=O)NC4(CCN(c5cc(Cl)ncn5)CC4)C3)c(Cl)c2)CC1] | 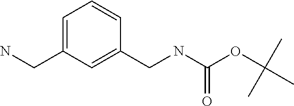 [SMILES: CC(C)(C)OC(=O)NCc1cccc(CN)c1] |
| | | 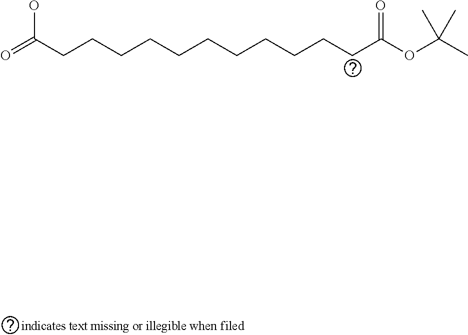 [SMILES: CC(C)(C)OC(=O)CCCCCCCCCCCC(=O)O] | 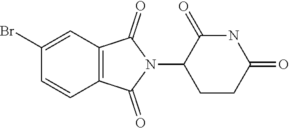 [SMILES: O=C1CCC(N2C(=O)c3ccc(Br)cc3C2=O)C(=O)N1] |
| 241 | 5 | 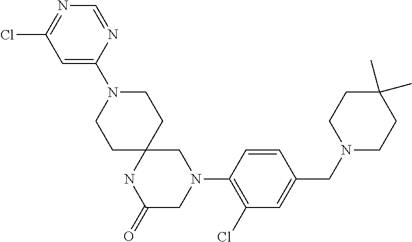 [SMILES: CC1(C)CCN(Cc2ccc(N3CC(=O)NC4(CCN(c5cc(Cl)ncn5)CC4)C3)c(Cl)c2)CC1] | 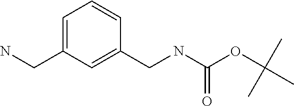 [SMILES: CC(C)(C)OC(=O)NCc1cccc(CN)c1] |
| | | 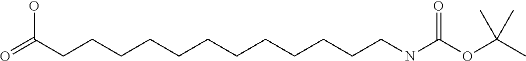 [SMILES: CC(C)(C)OC(=O)NCCCCCCCCCCCCC(=O)O] | 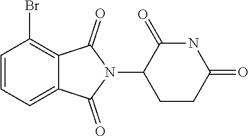 [SMILES: O=C1CCC(N2C(=O)c3cccc(Br)c3C2=O)C(=O)N1] |
| 242 | 5 | 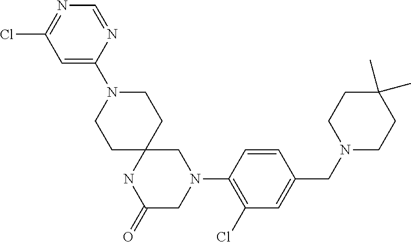 [SMILES: CC1(C)CCN(Cc2ccc(N3CC(=O)NC4(CCN(c5cc(Cl)ncn5)CC4)C3)c(Cl)c2)CC1] | 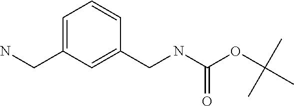 [SMILES: CC(C)(C)OC(=O)NCc1cccc(CN)c1] |
| | | 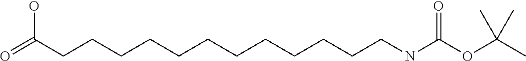 [SMILES: CC(C)(C)OC(=O)NCCCCCCCCCCCCC(=O)O] | 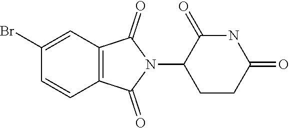 [SMILES: O=C1CCC(N2C(=O)c3ccc(Br)cc3C2=O)C(=O)N1] |
| 243 | 5 | 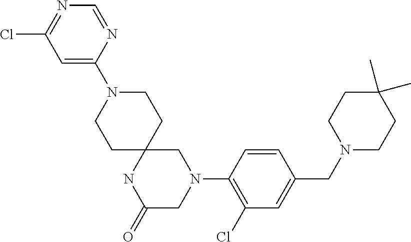 [SMILES: CC1(C)CCN(Cc2ccc(N3CC(=O)NC4(CCN(c5cc(Cl)ncn5)CC4)C3)c(Cl)c2)CC1] | 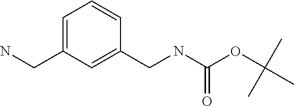 [SMILES: CC(C)(C)OC(=O)NCc1cccc(CN)c1] |
| | | 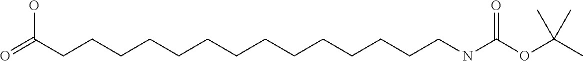 [SMILES: CC(C)(C)OC(=O)NCCCCCCCCCCCCCCC(=O)O] | 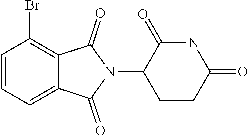 [SMILES: O=C1CCC(N2C(=O)c3cccc(Br)c3C2=O)C(=O)N1] |
| 244 | 5 | 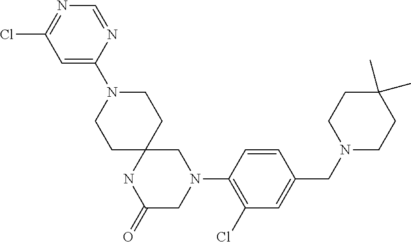 [SMILES: CC1(C)CCN(Cc2ccc(N3CC(=O)NC4(CCN(c5cc(Cl)ncn5)CC4)C3)c(Cl)c2)CC1] | 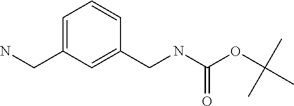 [SMILES: CC(C)(C)OC(=O)NCc1cccc(CN)c1] |
| | | 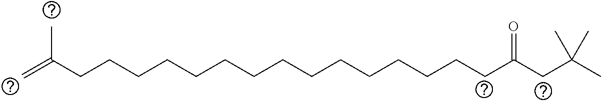 [SMILES: C=C(C)CCCCCCCCCCCCCCCC(=O)CC(C)(C)C] | 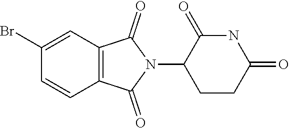 [SMILES: O=C1CCC(N2C(=O)c3ccc(Br)cc3C2=O)C(=O)N1] |
| 245 | 5 | 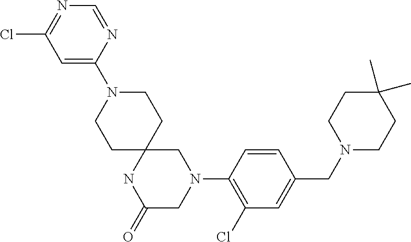 [SMILES: CC1(C)CCN(Cc2ccc(N3CC(=O)NC4(CCN(c5cc(Cl)ncn5)CC4)C3)c(Cl)c2)CC1] | 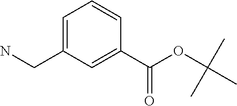 |
| | | 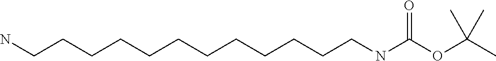 [SMILES: CC(C)(C)OC(=O)NCCCCCCCCCCCCN] | 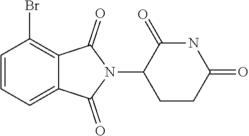 [SMILES: O=C1CCC(N2C(=O)c3cccc(Br)c3C2=O)C(=O)N1] |
| 246 | 5 | 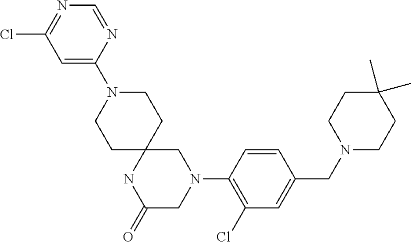 [SMILES: CC1(C)CCN(Cc2ccc(N3CC(=O)NC4(CCN(c5cc(Cl)ncn5)CC4)C3)c(Cl)c2)CC1] | 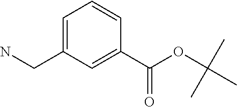 |
| | | 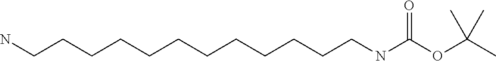 [SMILES: CC(C)(C)OC(=O)NCCCCCCCCCCCCN] | 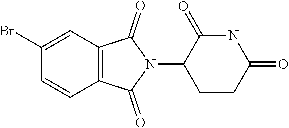 [SMILES: O=C1CCC(N2C(=O)c3ccc(Br)cc3C2=O)C(=O)N1] |
| 247 | 5 | 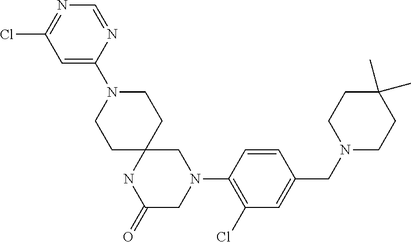 [SMILES: CC1(C)CCN(Cc2ccc(N3CC(=O)NC4(CCN(c5cc(Cl)ncn5)CC4)C3)c(Cl)c2)CC1] | 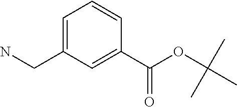 |
| | | 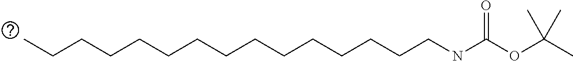 [SMILES: CCCCCCCCCCCCCCCNC(=O)OC(C)(C)C] | 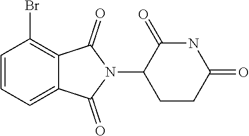 [SMILES: O=C1CCC(N2C(=O)c3cccc(Br)c3C2=O)C(=O)N1] |
| 248 | 5 | 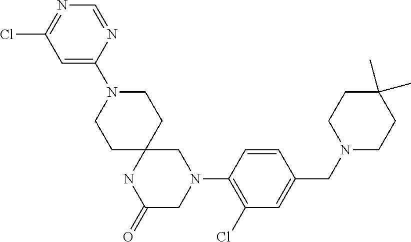 [SMILES: CC1(C)CCN(Cc2ccc(N3CC(=O)NC4(CCN(c5cc(Cl)ncn5)CC4)C3)c(Cl)c2)CC1] | 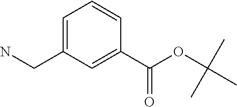 |
| | | 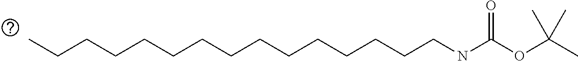 [SMILES: CCCCCCCCCCCCCCCNC(=O)OC(C)(C)C] | 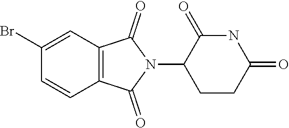 [SMILES: O=C1CCC(N2C(=O)c3ccc(Br)cc3C2=O)C(=O)N1] |
| 249 | 5 | 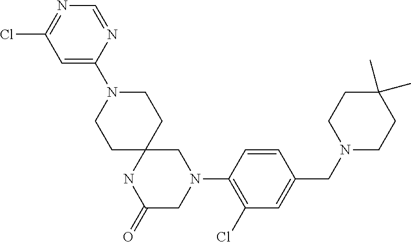 [SMILES: CC1(C)CCN(Cc2ccc(N3CC(=O)NC4(CCN(c5cc(Cl)ncn5)CC4)C3)c(Cl)c2)CC1] | 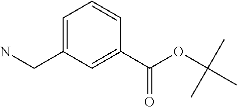 |
| | | 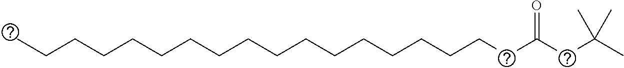 [SMILES: CCCCCCCCCCCCCCCCCCC(=O)CC(C)(C)C] | 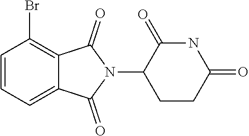 [SMILES: O=C1CCC(N2C(=O)c3cccc(Br)c3C2=O)C(=O)N1] |
| 250 | 5 | 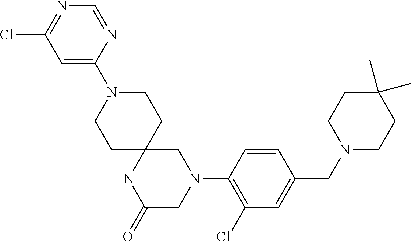 [SMILES: CC1(C)CCN(Cc2ccc(N3CC(=O)NC4(CCN(c5cc(Cl)ncn5)CC4)C3)c(Cl)c2)CC1] | 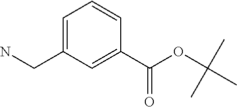 |
| | | 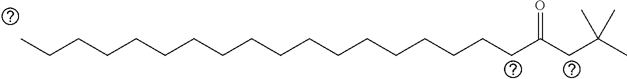 [SMILES: CCCCCCCCCCCCCCCCCCC(=O)CC(C)(C)C] | 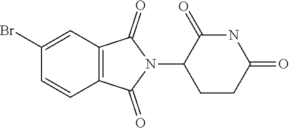 [SMILES: O=C1CCC(N2C(=O)c3ccc(Br)cc3C2=O)C(=O)N1] |
| 251 | 5 | 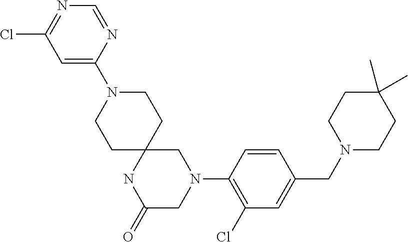 [SMILES: CC1(C)CCN(Cc2ccc(N3CC(=O)NC4(CCN(c5cc(Cl)ncn5)CC4)C3)c(Cl)c2)CC1] | 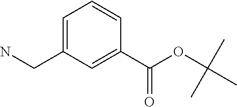 |
| | | 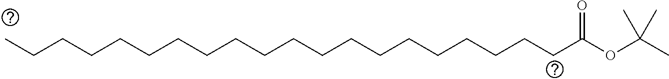 [SMILES: CCCCCCCCCCCCCCCCCCCCC(=O)OC(C)(C)C] | 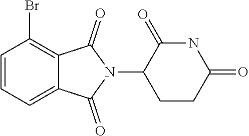 [SMILES: O=C1CCC(N2C(=O)c3cccc(Br)c3C2=O)C(=O)N1] |
| 252 | 5 | 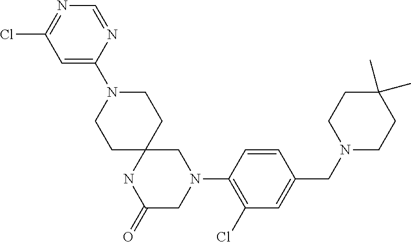 [SMILES: CC1(C)CCN(Cc2ccc(N3CC(=O)NC4(CCN(c5cc(Cl)ncn5)CC4)C3)c(Cl)c2)CC1] | 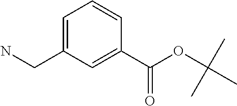 |
| | | 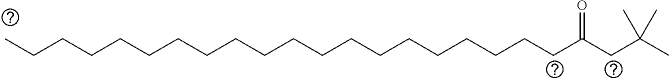 [SMILES: CCCCCCCCCCCCCCCCCCCCC(=O)CC(C)(C)C] | 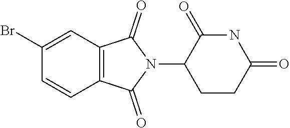 [SMILES: O=C1CCC(N2C(=O)c3ccc(Br)cc3C2=O)C(=O)N1] |
| 253 | 5 | 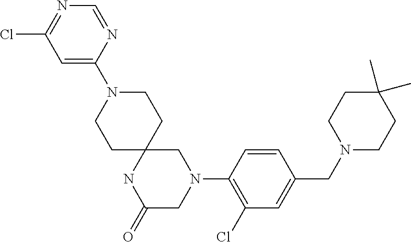 [SMILES: CC1(C)CCN(Cc2ccc(N3CC(=O)NC4(CCN(c5cc(Cl)ncn5)CC4)C3)c(Cl)c2)CC1] | 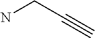 |
| | | 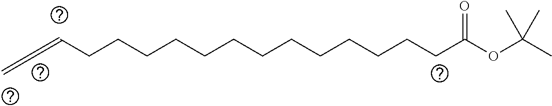 [SMILES: C=C=CCCCCCCCCCCCCCC(=O)OC(C)(C)C] | 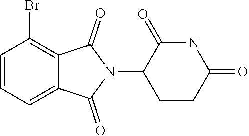 [SMILES: O=C1CCC(N2C(=O)c3cccc(Br)c3C2=O)C(=O)N1] |
| 254 | 5 | 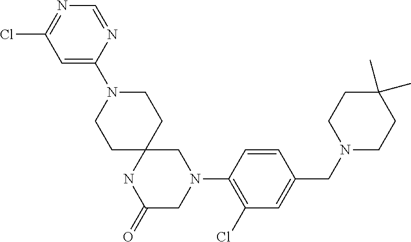 [SMILES: CC1(C)CCN(Cc2ccc(N3CC(=O)NC4(CCN(c5cc(Cl)ncn5)CC4)C3)c(Cl)c2)CC1] | 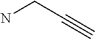 |
| | | 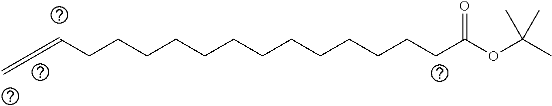 [SMILES: C=C=CCCCCCCCCCCCCCC(=O)OC(C)(C)C] | 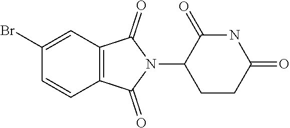 [SMILES: O=C1CCC(N2C(=O)c3ccc(Br)cc3C2=O)C(=O)N1] |
| 255 | 5 | 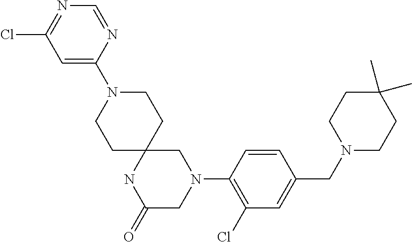 [SMILES: CC1(C)CCN(Cc2ccc(N3CC(=O)NC4(CCN(c5cc(Cl)ncn5)CC4)C3)c(Cl)c2)CC1] | 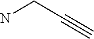 |
| | | 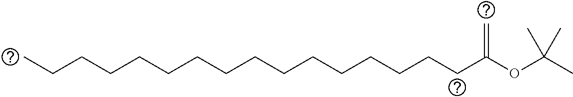 [SMILES: C=C(CCCCCCCCCCCCCCCC)OC(C)(C)C] | 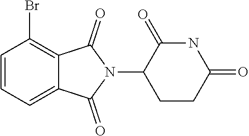 [SMILES: O=C1CCC(N2C(=O)c3cccc(Br)c3C2=O)C(=O)N1] |
| 256 | 5 | 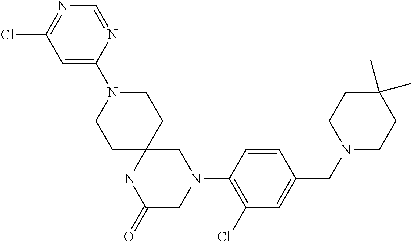 [SMILES: CC1(C)CCN(Cc2ccc(N3CC(=O)NC4(CCN(c5cc(Cl)ncn5)CC4)C3)c(Cl)c2)CC1] | 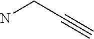 |
| | | 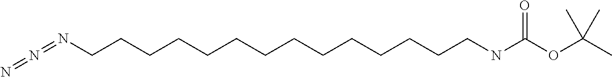 | 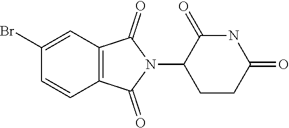 [SMILES: O=C1CCC(N2C(=O)c3ccc(Br)cc3C2=O)C(=O)N1] |
| 257 | 5 | 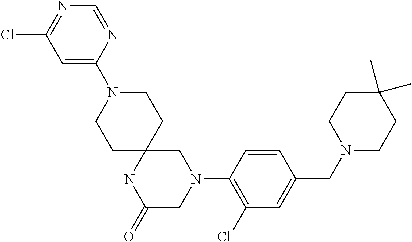 [SMILES: CC1(C)CCN(Cc2ccc(N3CC(=O)NC4(CCN(c5cc(Cl)ncn5)CC4)C3)c(Cl)c2)CC1] | 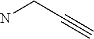 |
| | | 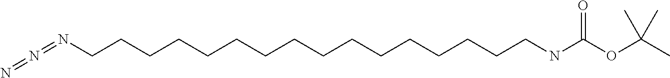 | 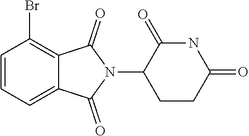 [SMILES: O=C1CCC(N2C(=O)c3cccc(Br)c3C2=O)C(=O)N1] |
| 258 | 5 | 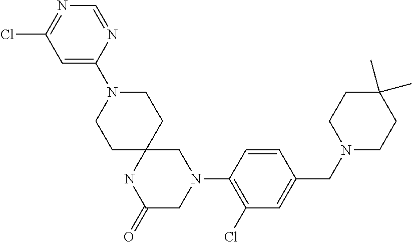 [SMILES: CC1(C)CCN(Cc2ccc(N3CC(=O)NC4(CCN(c5cc(Cl)ncn5)CC4)C3)c(Cl)c2)CC1] | 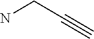 |
| | | 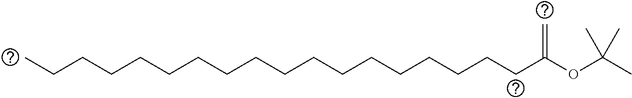 [SMILES: C=C(CCCCCCCCCCCCCCCCCC)OC(C)(C)C] | 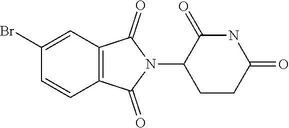 [SMILES: O=C1CCC(N2C(=O)c3ccc(Br)cc3C2=O)C(=O)N1] |
| 259 | 5 | 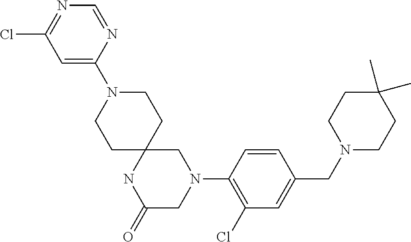 [SMILES: CC1(C)CCN(Cc2ccc(N3CC(=O)NC4(CCN(c5cc(Cl)ncn5)CC4)C3)c(Cl)c2)CC1] | 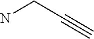 |
| | | 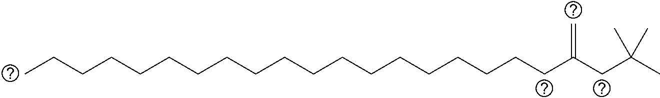 [SMILES: C=C(CCCCCCCCCCCCCCCCCCC)CC(C)(C)C] | 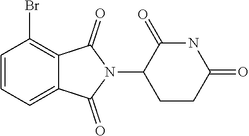 [SMILES: O=C1CCC(N2C(=O)c3cccc(Br)c3C2=O)C(=O)N1] |
| 260 | 5 | 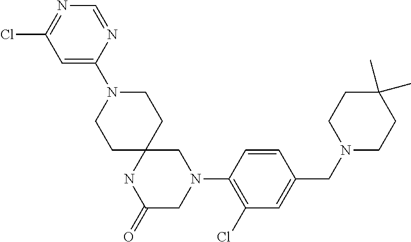 [SMILES: CC1(C)CCN(Cc2ccc(N3CC(=O)NC4(CCN(c5cc(Cl)ncn5)CC4)C3)c(Cl)c2)CC1] | 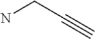 |
| | | 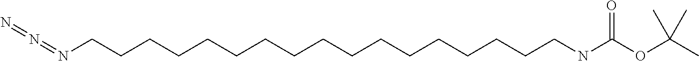 | 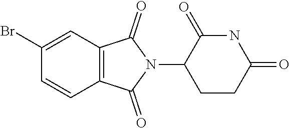 [SMILES: O=C1CCC(N2C(=O)c3ccc(Br)cc3C2=O)C(=O)N1] |
| 261 | 6 | 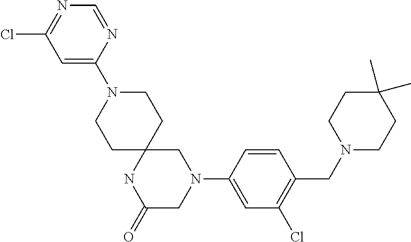 [SMILES: CC1(C)CCN(Cc2ccc(N3CC(=O)NC4(CCN(c5cc(Cl)ncn5)CC4)C3)cc2Cl)CC1] | 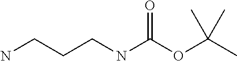 |
| | | 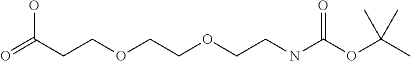 [SMILES: CC(C)(C)OC(=O)NCCOCCOCCC(=O)O] | 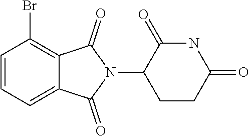 [SMILES: O=C1CCC(N2C(=O)c3cccc(Br)c3C2=O)C(=O)N1] |
| 262 | 6 | 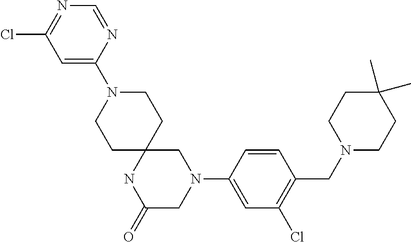 [SMILES: CC1(C)CCN(Cc2ccc(N3CC(=O)NC4(CCN(c5cc(Cl)ncn5)CC4)C3)cc2Cl)CC1] | 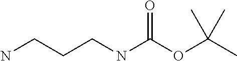 |
| | | 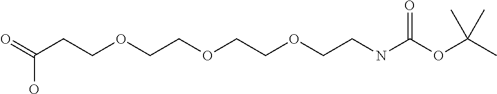 [SMILES: CC(C)(C)OC(=O)NCCOCCOCCOCCC(=O)O] | 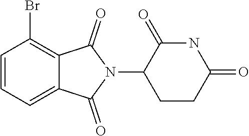 [SMILES: O=C1CCC(N2C(=O)c3cccc(Br)c3C2=O)C(=O)N1] |
| 263 | 6 | 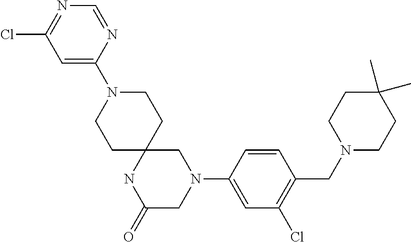 [SMILES: CC1(C)CCN(Cc2ccc(N3CC(=O)NC4(CCN(c5cc(Cl)ncn5)CC4)C3)cc2Cl)CC1] | 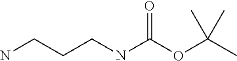 |
| | | 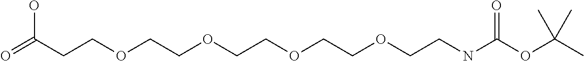 [SMILES: CC(C)(C)OC(=O)NCCOCCOCCOCCOCCC(=O)O] | 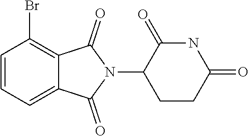 [SMILES: O=C1CCC(N2C(=O)c3cccc(Br)c3C2=O)C(=O)N1] |
| 264 | 6 | 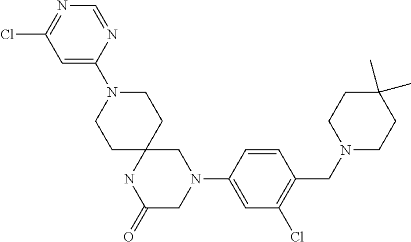 [SMILES: CC1(C)CCN(Cc2ccc(N3CC(=O)NC4(CCN(c5cc(Cl)ncn5)CC4)C3)cc2Cl)CC1] | 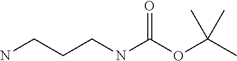 |
| | | 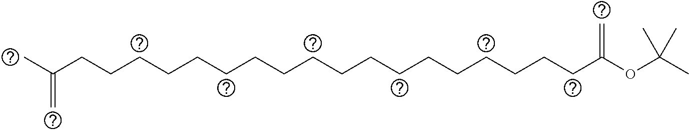 [SMILES: C=C(C)CCCCCCCCCCCCCCCCCCC(=C)OC(C)(C)C] | 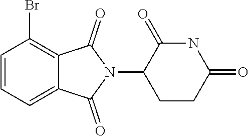 [SMILES: O=C1CCC(N2C(=O)c3cccc(Br)c3C2=O)C(=O)N1] |
| 265 | 6 | 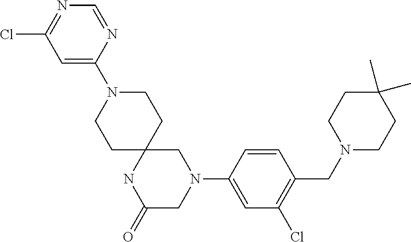 [SMILES: CC1(C)CCN(Cc2ccc(N3CC(=O)NC4(CCN(c5cc(Cl)ncn5)CC4)C3)cc2Cl)CC1] | 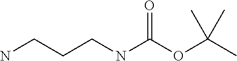 |
| | | 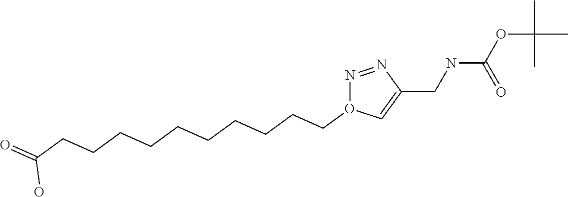 | 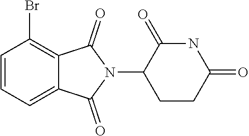 [SMILES: O=C1CCC(N2C(=O)c3cccc(Br)c3C2=O)C(=O)N1] |
| 266 | 6 | 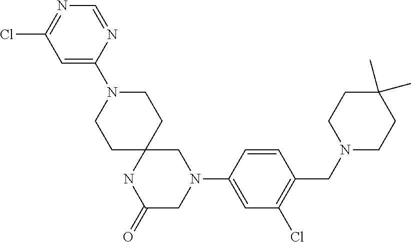 [SMILES: CC1(C)CCN(Cc2ccc(N3CC(=O)NC4(CCN(c5cc(Cl)ncn5)CC4)C3)cc2Cl)CC1] | 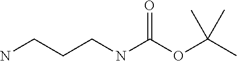 |
| | | 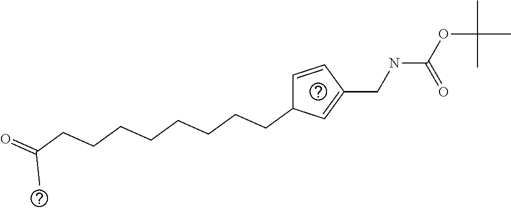 [SMILES: CC(=O)CCCCCCCCC1C=CC(CNC(=O)OC(C)(C)C)=C1] | 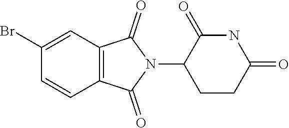 [SMILES: O=C1CCC(N2C(=O)c3ccc(Br)cc3C2=O)C(=O)N1] |
| 267 | 6 | 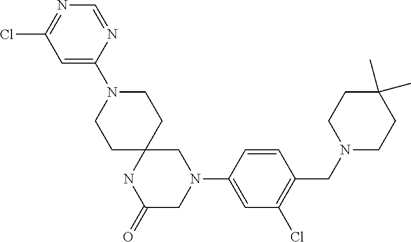 [SMILES: CC1(C)CCN(Cc2ccc(N3CC(=O)NC4(CCN(c5cc(Cl)ncn5)CC4)C3)cc2Cl)CC1] | 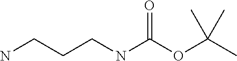 |
| | | 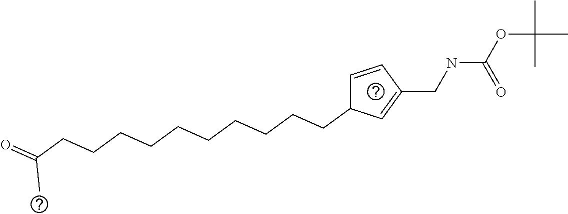 [SMILES: CC(=O)CCCCCCCCCCC1C=CC(CNC(=O)OC(C)(C)C)=C1] | 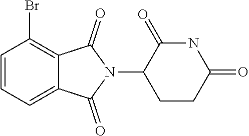 [SMILES: O=C1CCC(N2C(=O)c3cccc(Br)c3C2=O)C(=O)N1] |
| 268 | 6 | 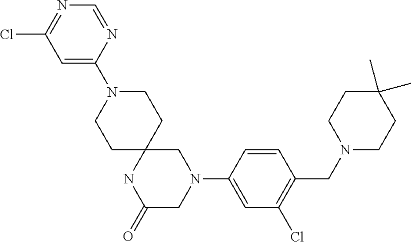 [SMILES: CC1(C)CCN(Cc2ccc(N3CC(=O)NC4(CCN(c5cc(Cl)ncn5)CC4)C3)cc2Cl)CC1] | 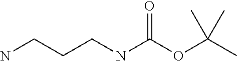 |
| | | 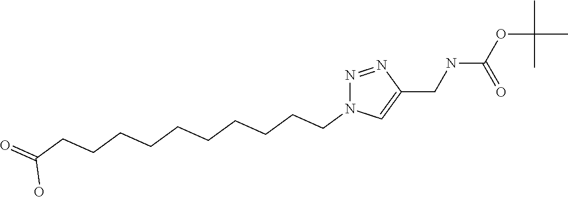 [SMILES: CC(C)(C)OC(=O)NCc1cn(CCCCCCCCCCC(=O)O)nn1] | 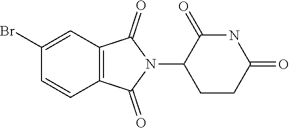 [SMILES: O=C1CCC(N2C(=O)c3ccc(Br)cc3C2=O)C(=O)N1] |
| 269 | 6 | 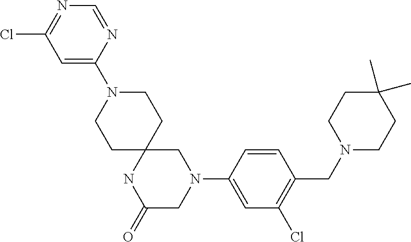 [SMILES: CC1(C)CCN(Cc2ccc(N3CC(=O)NC4(CCN(c5cc(Cl)ncn5)CC4)C3)cc2Cl)CC1] | 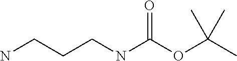 |
| | | 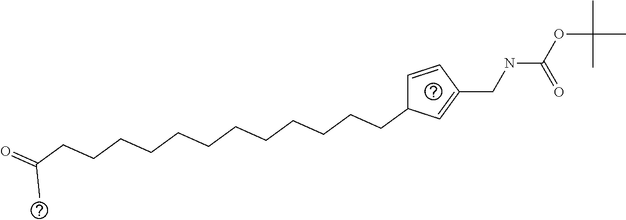 [SMILES: CC(=O)CCCCCCCCCCCCC1C=CC(CNC(=O)OC(C)(C)C)=C1] | 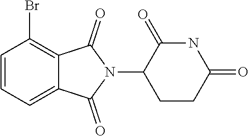 [SMILES: O=C1CCC(N2C(=O)c3cccc(Br)c3C2=O)C(=O)N1] |
| 270 | 6 | 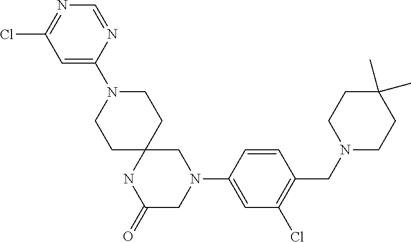 [SMILES: CC1(C)CCN(Cc2ccc(N3CC(=O)NC4(CCN(c5cc(Cl)ncn5)CC4)C3)cc2Cl)CC1] | 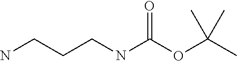 |
| | | 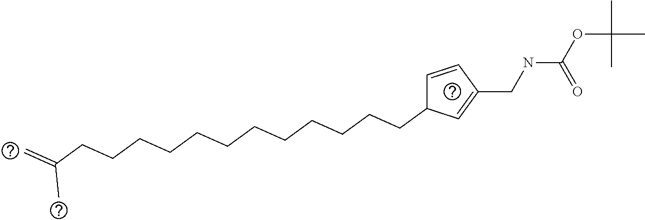 [SMILES: C=C(C)CCCCCCCCCCCCC1C=CC(CNC(=O)OC(C)(C)C)=C1] | 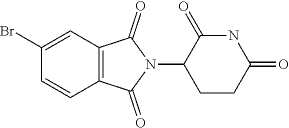 [SMILES: O=C1CCC(N2C(=O)c3ccc(Br)cc3C2=O)C(=O)N1] |
| 271 | 6 | 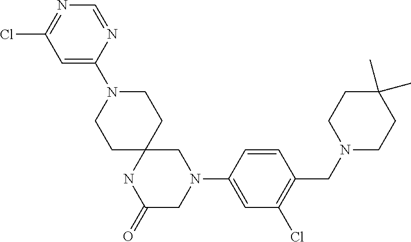 [SMILES: CC1(C)CCN(Cc2ccc(N3CC(=O)NC4(CCN(c5cc(Cl)ncn5)CC4)C3)cc2Cl)CC1] | 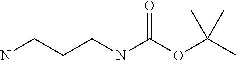 |
| | | 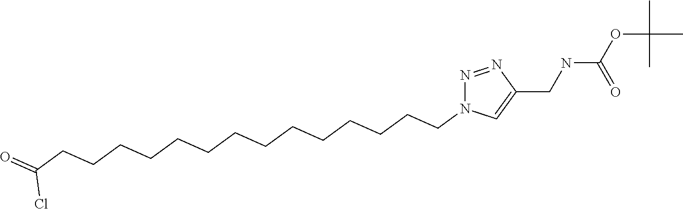 [SMILES: CC(C)(C)OC(=O)NCc1cn(CCCCCCCCCCCCCCC(=O)Cl)nn1] | 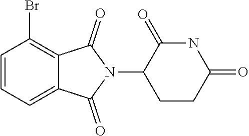 [SMILES: O=C1CCC(N2C(=O)c3cccc(Br)c3C2=O)C(=O)N1] |
| 272 | 6 | 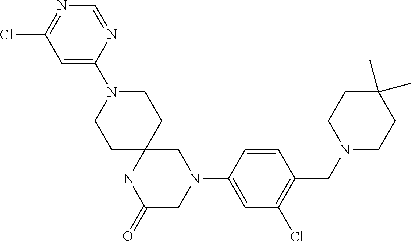 [SMILES: CC1(C)CCN(Cc2ccc(N3CC(=O)NC4(CCN(c5cc(Cl)ncn5)CC4)C3)cc2Cl)CC1] | 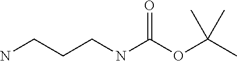 |
| | | 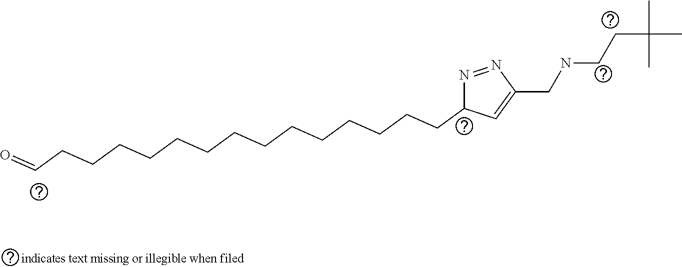 [SMILES: CC(C)(C)CCNCC1=CC(CCCCCCCCCCCCCCC=O)N=N1] | 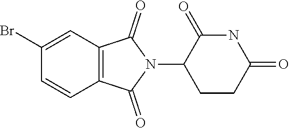 [SMILES: O=C1CCC(N2C(=O)c3ccc(Br)cc3C2=O)C(=O)N1] |
| 273 | 6 | 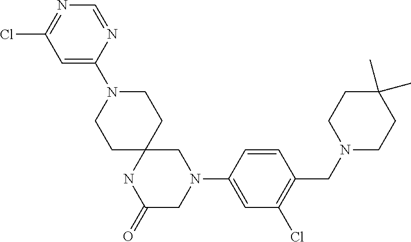 [SMILES: CC1(C)CCN(Cc2ccc(N3CC(=O)NC4(CCN(c5cc(Cl)ncn5)CC4)C3)cc2Cl)CC1] | 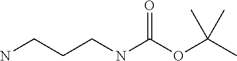 |
| | | 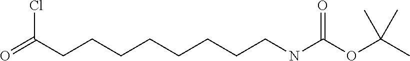 [SMILES: CC(C)(C)OC(=O)NCCCCCCCCC(=O)Cl] | 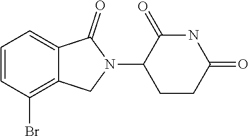 [SMILES: O=C1CCC(N2Cc3c(Br)cccc3C2=O)C(=O)N1] |
| 274 | 6 | 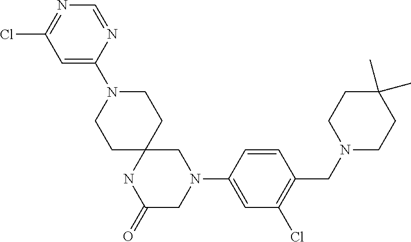 [SMILES: CC1(C)CCN(Cc2ccc(N3CC(=O)NC4(CCN(c5cc(Cl)ncn5)CC4)C3)cc2Cl)CC1] | 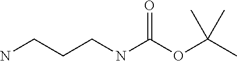 |
| | | 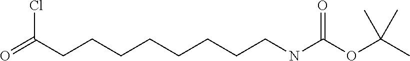 [SMILES: CC(C)(C)OC(=O)NCCCCCCCCC(=O)Cl] | 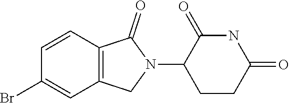 [SMILES: O=C1CCC(N2Cc3cc(Br)ccc3C2=O)C(=O)N1] |
| 275 | 6 | 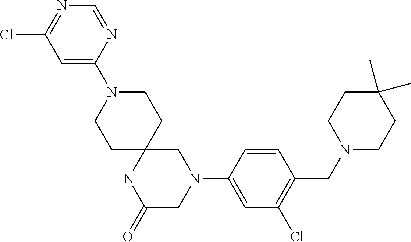 [SMILES: CC1(C)CCN(Cc2ccc(N3CC(=O)NC4(CCN(c5cc(Cl)ncn5)CC4)C3)cc2Cl)CC1] | 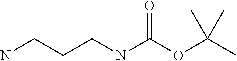 |
| | | 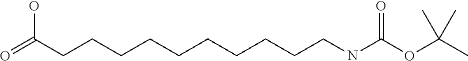 [SMILES: CC(C)(C)OC(=O)NCCCCCCCCCCC(=O)O] | 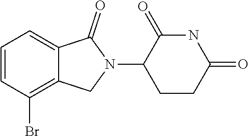 [SMILES: O=C1CCC(N2Cc3c(Br)cccc3C2=O)C(=O)N1] |
| 276 | 6 | 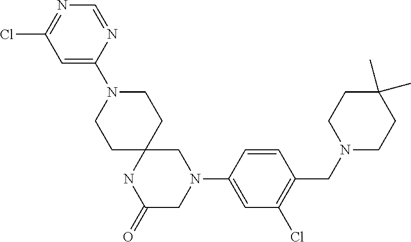 [SMILES: CC1(C)CCN(Cc2ccc(N3CC(=O)NC4(CCN(c5cc(Cl)ncn5)CC4)C3)cc2Cl)CC1] | 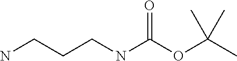 |
| | | 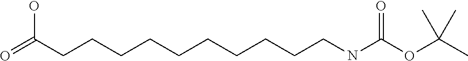 [SMILES: CC(C)(C)OC(=O)NCCCCCCCCCCC(=O)O] | 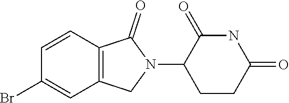 [SMILES: O=C1CCC(N2Cc3cc(Br)ccc3C2=O)C(=O)N1] |
| 277 | 6 | 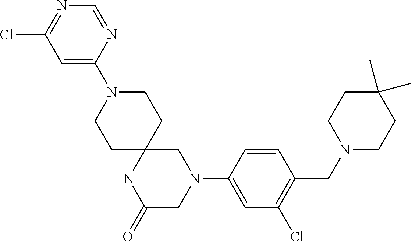 [SMILES: CC1(C)CCN(Cc2ccc(N3CC(=O)NC4(CCN(c5cc(Cl)ncn5)CC4)C3)cc2Cl)CC1] | 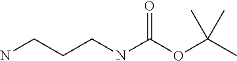 |
| | | 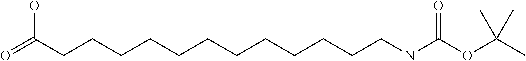 [SMILES: CC(C)(C)OC(=O)NCCCCCCCCCCCCC(=O)O] | 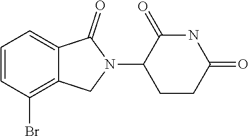 [SMILES: O=C1CCC(N2Cc3c(Br)cccc3C2=O)C(=O)N1] |
| 278 | 6 | 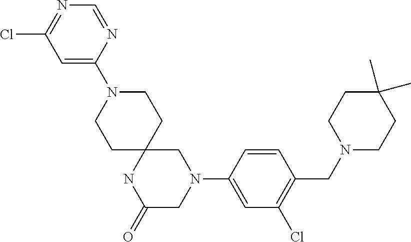 [SMILES: CC1(C)CCN(Cc2ccc(N3CC(=O)NC4(CCN(c5cc(Cl)ncn5)CC4)C3)cc2Cl)CC1] | 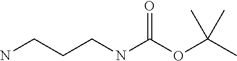 |
| | | 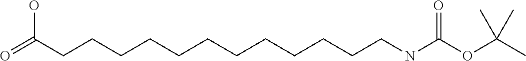 [SMILES: CC(C)(C)OC(=O)NCCCCCCCCCCCCC(=O)O] | 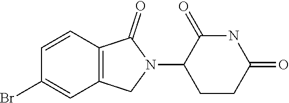 [SMILES: O=C1CCC(N2Cc3cc(Br)ccc3C2=O)C(=O)N1] |
| 279 | 6 | 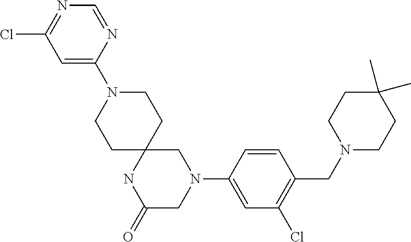 [SMILES: CC1(C)CCN(Cc2ccc(N3CC(=O)NC4(CCN(c5cc(Cl)ncn5)CC4)C3)cc2Cl)CC1] | 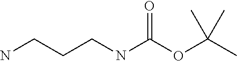 |
| | | 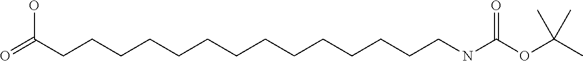 [SMILES: CC(C)(C)OC(=O)NCCCCCCCCCCCCCCC(=O)O] | 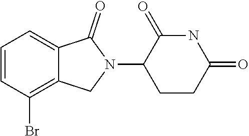 [SMILES: O=C1CCC(N2Cc3c(Br)cccc3C2=O)C(=O)N1] |
| 280 | 6 | 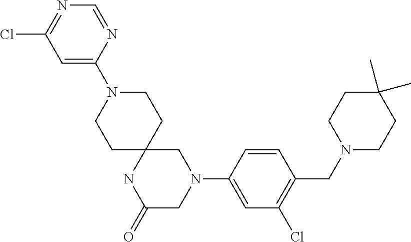 [SMILES: CC1(C)CCN(Cc2ccc(N3CC(=O)NC4(CCN(c5cc(Cl)ncn5)CC4)C3)cc2Cl)CC1] | 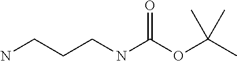 |
| | | 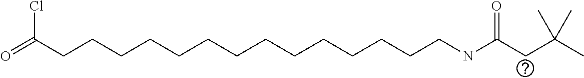 [SMILES: CC(C)(C)CC(=O)NCCCCCCCCCCCCCCC(=O)Cl] | 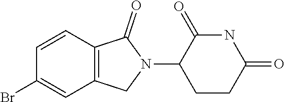 [SMILES: O=C1CCC(N2Cc3cc(Br)ccc3C2=O)C(=O)N1] |
| 281 | 6 | 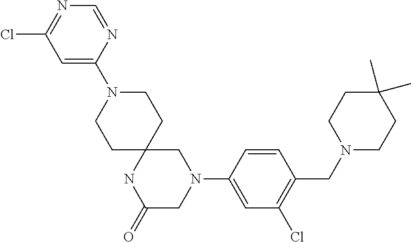 [SMILES: CC1(C)CCN(Cc2ccc(N3CC(=O)NC4(CCN(c5cc(Cl)ncn5)CC4)C3)cc2Cl)CC1] | 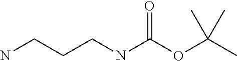 |
| | | 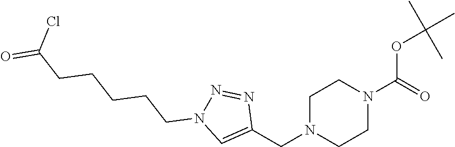 [SMILES: CC(C)(C)OC(=O)N1CCN(Cc2cn(CCCCCC(=O)Cl)nn2)CC1] | 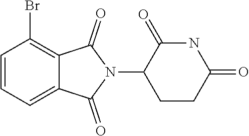 [SMILES: O=C1CCC(N2C(=O)c3cccc(Br)c3C2=O)C(=O)N1] |
| 282 | 6 | 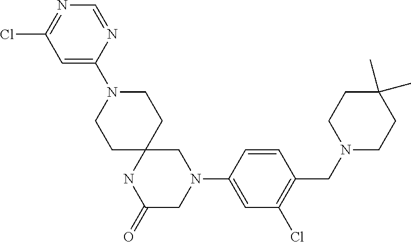 [SMILES: CC1(C)CCN(Cc2ccc(N3CC(=O)NC4(CCN(c5cc(Cl)ncn5)CC4)C3)cc2Cl)CC1] | 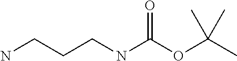 |
| | | 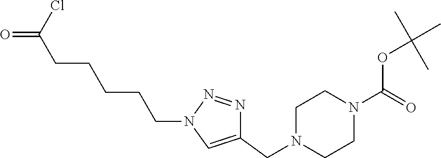 [SMILES: CC(C)(C)OC(=O)N1CCN(Cc2cn(CCCCCC(=O)Cl)nn2)CC1] | 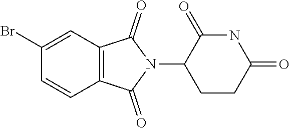 [SMILES: O=C1CCC(N2C(=O)c3ccc(Br)cc3C2=O)C(=O)N1] |
| 283 | 6 | 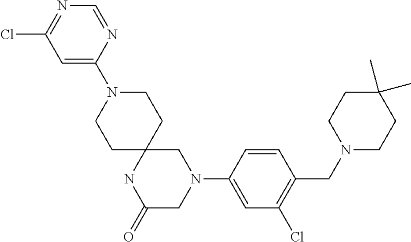 [SMILES: CC1(C)CCN(Cc2ccc(N3CC(=O)NC4(CCN(c5cc(Cl)ncn5)CC4)C3)cc2Cl)CC1] | 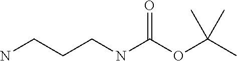 |
| | | 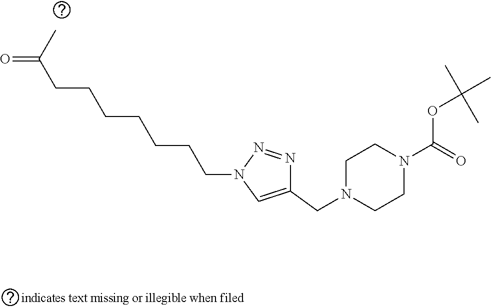 [SMILES: CC(=O)CCCCCCCn1cc(CN2CCN(C(=O)OC(C)(C)C)CC2)nn1] | 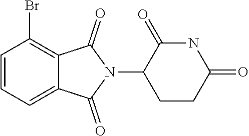 [SMILES: O=C1CCC(N2C(=O)c3cccc(Br)c3C2=O)C(=O)N1] |
| 284 | 6 | 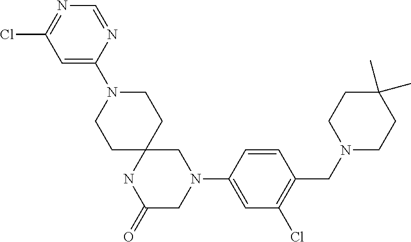 [SMILES: CC1(C)CCN(Cc2ccc(N3CC(=O)NC4(CCN(c5cc(Cl)ncn5)CC4)C3)cc2Cl)CC1] | 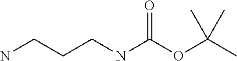 |
| | | 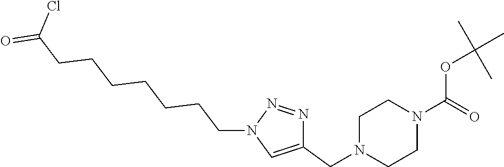 [SMILES: CC(C)(C)OC(=O)N1CCN(Cc2cn(CCCCCCCC(=O)Cl)nn2)CC1] | 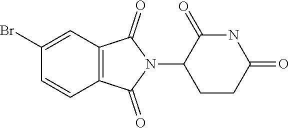 [SMILES: O=C1CCC(N2C(=O)c3ccc(Br)cc3C2=O)C(=O)N1] |
| 285 | 6 | 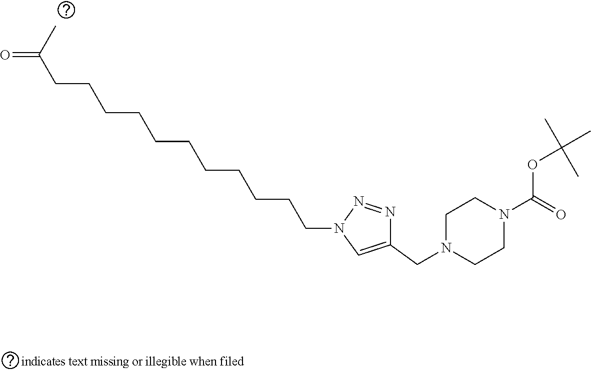 [SMILES: CC(=O)CCCCCCCCCCCn1cc(CN2CCN(C(=O)OC(C)(C)C)CC2)nn1] | 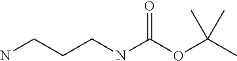 |
| | | 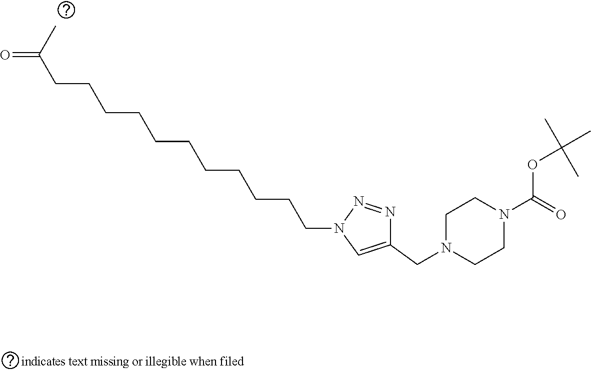 [SMILES: CC(=O)CCCCCCCCCCCn1cc(CN2CCN(C(=O)OC(C)(C)C)CC2)nn1] | 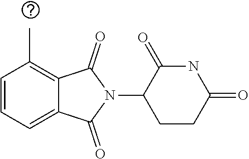 [SMILES: Cc1cccc2c1C(=O)N(C1CCC(=O)NC1=O)C2=O] |
| 286 | 6 | 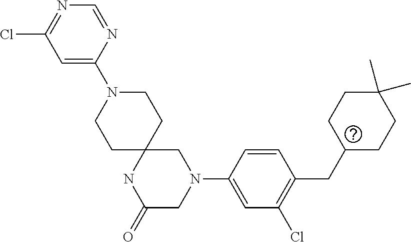 [SMILES: CC1(C)CCC(Cc2ccc(N3CC(=O)NC4(CCN(c5cc(Cl)ncn5)CC4)C3)cc2Cl)CC1] | 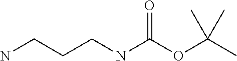 |
| | | 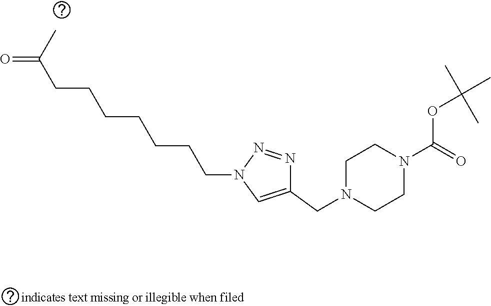 [SMILES: CC(=O)CCCCCCCn1cc(CN2CCN(C(=O)OC(C)(C)C)CC2)nn1] | 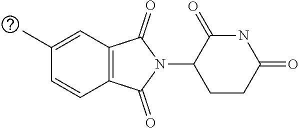 [SMILES: Cc1ccc2c(c1)C(=O)N(C1CCC(=O)NC1=O)C2=O] |
| 287 | 6 | 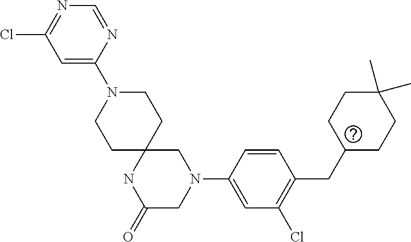 [SMILES: CC1(C)CCC(Cc2ccc(N3CC(=O)NC4(CCN(c5cc(Cl)ncn5)CC4)C3)cc2Cl)CC1] | 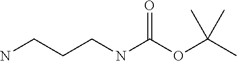 |
| | | 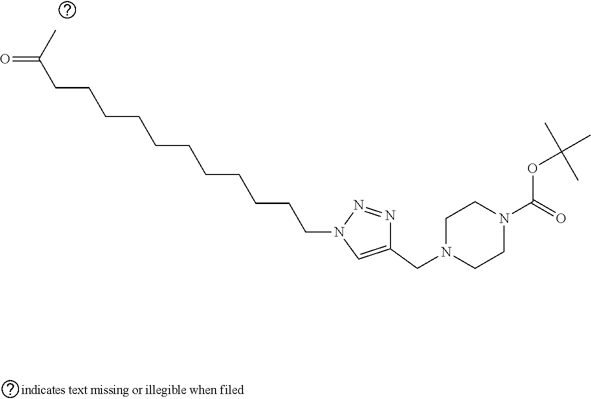 [SMILES: CC(=O)CCCCCCCCCCCn1cc(CN2CCN(C(=O)OC(C)(C)C)CC2)nn1] | 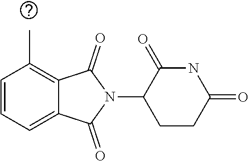 [SMILES: Cc1cccc2c1C(=O)N(C1CCC(=O)NC1=O)C2=O] |
| 288 | 6 | 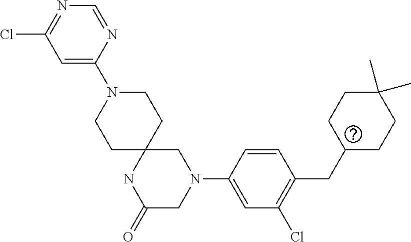 [SMILES: CC1(C)CCC(Cc2ccc(N3CC(=O)NC4(CCN(c5cc(Cl)ncn5)CC4)C3)cc2Cl)CC1] | 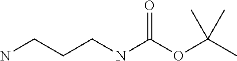 |
| | | 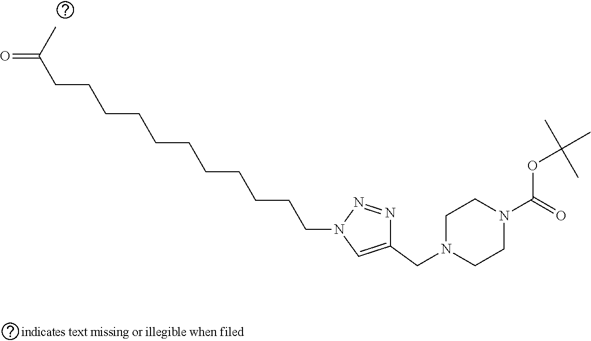 [SMILES: CC(=O)CCCCCCCCCCCn1cc(CN2CCN(C(=O)OC(C)(C)C)CC2)nn1] | 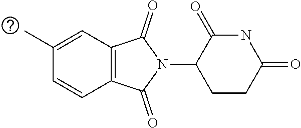 [SMILES: Cc1ccc2c(c1)C(=O)N(C1CCC(=O)NC1=O)C2=O] |
| 289 | 6 | 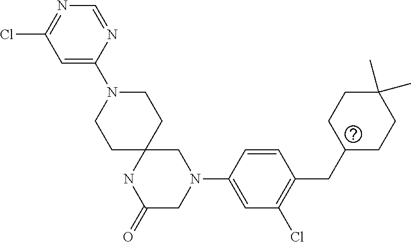 [SMILES: CC1(C)CCC(Cc2ccc(N3CC(=O)NC4(CCN(c5cc(Cl)ncn5)CC4)C3)cc2Cl)CC1] | 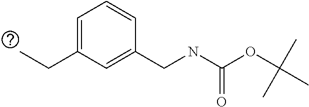 [SMILES: CCc1cccc(CNC(=O)OC(C)(C)C)c1] |
| | | 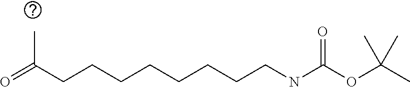 [SMILES: CC(=O)CCCCCCCCNC(=O)OC(C)(C)C] | 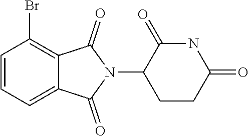 [SMILES: O=C1CCC(N2C(=O)c3cccc(Br)c3C2=O)C(=O)N1] |
| 290 | 6 | 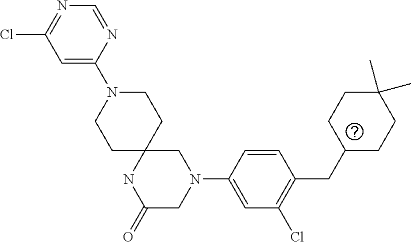 [SMILES: CC1(C)CCC(Cc2ccc(N3CC(=O)NC4(CCN(c5cc(Cl)ncn5)CC4)C3)cc2Cl)CC1] | 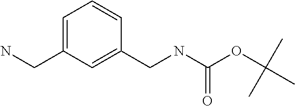 [SMILES: CC(C)(C)OC(=O)NCc1cccc(CN)c1] |
| | | 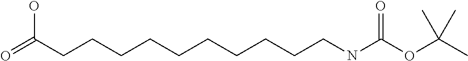 [SMILES: CC(C)(C)OC(=O)NCCCCCCCCCCC(=O)O] | 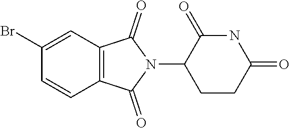 [SMILES: O=C1CCC(N2C(=O)c3ccc(Br)cc3C2=O)C(=O)N1] |
| 291 | 6 | 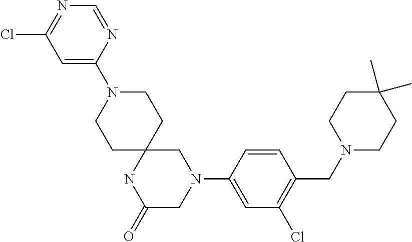 [SMILES: CC1(C)CCN(Cc2ccc(N3CC(=O)NC4(CCN(c5cc(Cl)ncn5)CC4)C3)cc2Cl)CC1] | 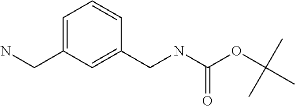 [SMILES: CC(C)(C)OC(=O)NCc1cccc(CN)c1] |
| | | 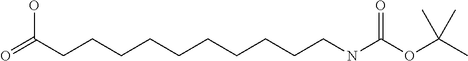 [SMILES: CC(C)(C)OC(=O)NCCCCCCCCCCC(=O)O] | 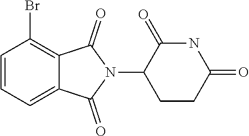 [SMILES: O=C1CCC(N2C(=O)c3cccc(Br)c3C2=O)C(=O)N1] |
| 292 | 6 | 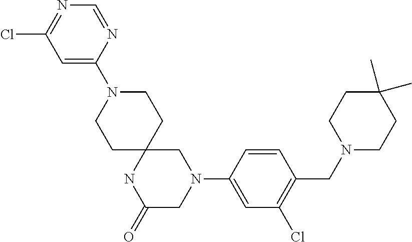 [SMILES: CC1(C)CCN(Cc2ccc(N3CC(=O)NC4(CCN(c5cc(Cl)ncn5)CC4)C3)cc2Cl)CC1] | 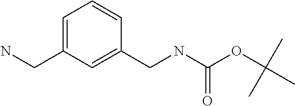 [SMILES: CC(C)(C)OC(=O)NCc1cccc(CN)c1] |
| | | 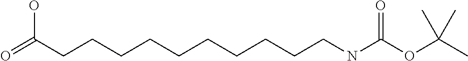 [SMILES: CC(C)(C)OC(=O)NCCCCCCCCCCC(=O)O] | 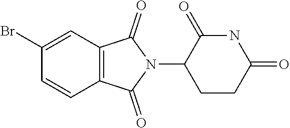 [SMILES: O=C1CCC(N2C(=O)c3ccc(Br)cc3C2=O)C(=O)N1] |
| 293 | 6 | 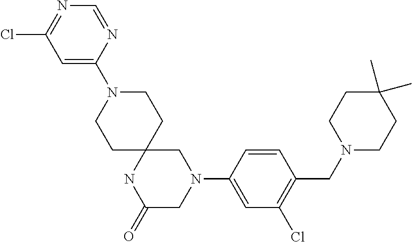 [SMILES: CC1(C)CCN(Cc2ccc(N3CC(=O)NC4(CCN(c5cc(Cl)ncn5)CC4)C3)cc2Cl)CC1] | 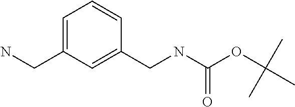 [SMILES: CC(C)(C)OC(=O)NCc1cccc(CN)c1] |
| | | 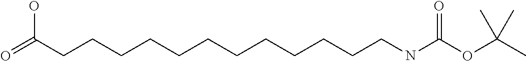 [SMILES: CC(C)(C)OC(=O)NCCCCCCCCCCCCC(=O)O] | 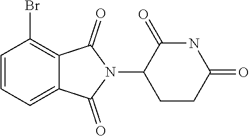 [SMILES: O=C1CCC(N2C(=O)c3cccc(Br)c3C2=O)C(=O)N1] |
| 294 | 6 | 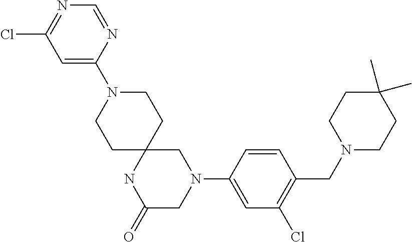 [SMILES: CC1(C)CCN(Cc2ccc(N3CC(=O)NC4(CCN(c5cc(Cl)ncn5)CC4)C3)cc2Cl)CC1] | 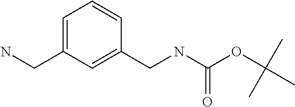 [SMILES: CC(C)(C)OC(=O)NCc1cccc(CN)c1] |
| | | 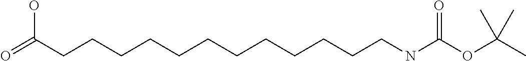 [SMILES: CC(C)(C)OC(=O)NCCCCCCCCCCCCC(=O)O] | 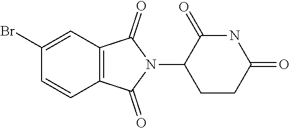 [SMILES: O=C1CCC(N2C(=O)c3ccc(Br)cc3C2=O)C(=O)N1] |
| 295 | 6 | 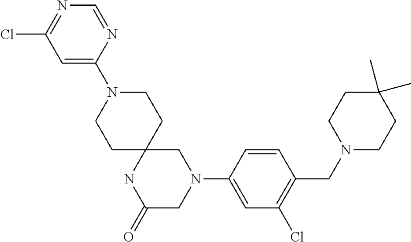 [SMILES: CC1(C)CCN(Cc2ccc(N3CC(=O)NC4(CCN(c5cc(Cl)ncn5)CC4)C3)cc2Cl)CC1] | 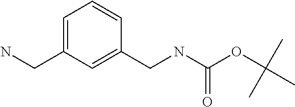 [SMILES: CC(C)(C)OC(=O)NCc1cccc(CN)c1] |
| | | 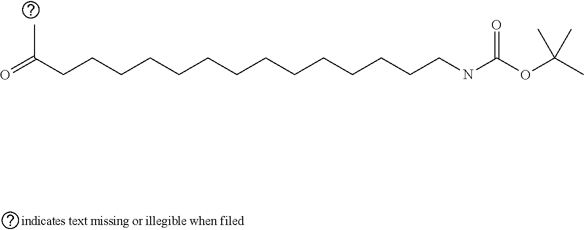 [SMILES: CC(=O)CCCCCCCCCCCCCCNC(=O)OC(C)(C)C] | 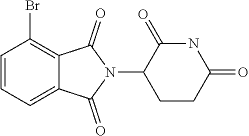 [SMILES: O=C1CCC(N2C(=O)c3cccc(Br)c3C2=O)C(=O)N1] |
| 296 | 6 | 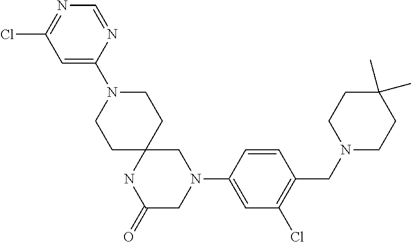 [SMILES: CC1(C)CCN(Cc2ccc(N3CC(=O)NC4(CCN(c5cc(Cl)ncn5)CC4)C3)cc2Cl)CC1] | 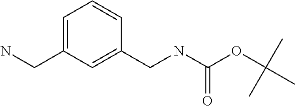 [SMILES: CC(C)(C)OC(=O)NCc1cccc(CN)c1] |
| | | 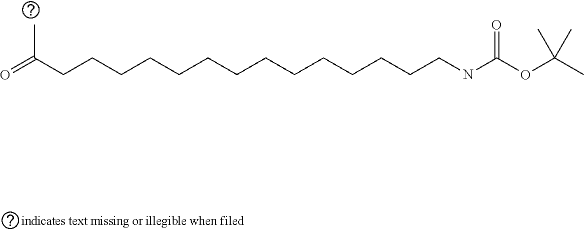 [SMILES: CC(=O)CCCCCCCCCCCCCCNC(=O)OC(C)(C)C] | 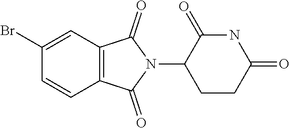 [SMILES: O=C1CCC(N2C(=O)c3ccc(Br)cc3C2=O)C(=O)N1] |
| 297 | 6 | 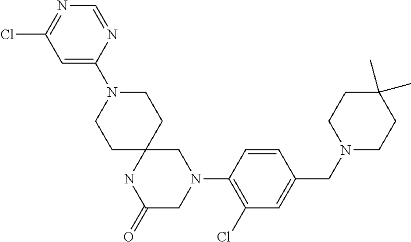 [SMILES: CC1(C)CCN(Cc2ccc(N3CC(=O)NC4(CCN(c5cc(Cl)ncn5)CC4)C3)c(Cl)c2)CC1] | 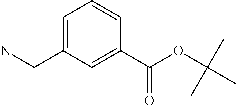 |
| | | 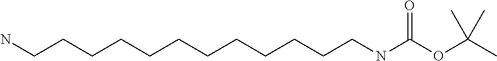 [SMILES: CC(C)(C)OC(=O)NCCCCCCCCCCCCN] | 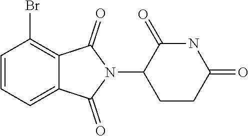 [SMILES: O=C1CCC(N2C(=O)c3cccc(Br)c3C2=O)C(=O)N1] |
| 298 | 6 | 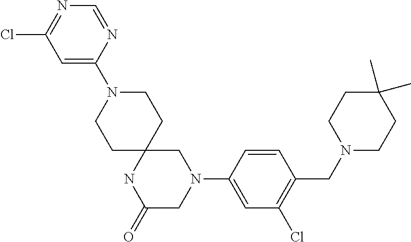 [SMILES: CC1(C)CCN(Cc2ccc(N3CC(=O)NC4(CCN(c5cc(Cl)ncn5)CC4)C3)cc2Cl)CC1] | 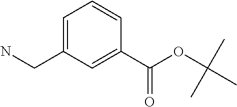 |
| | | 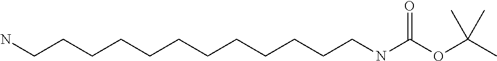 [SMILES: CC(C)(C)OC(=O)NCCCCCCCCCCCCN] | 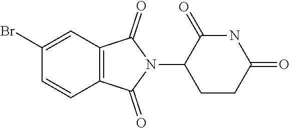 [SMILES: O=C1CCC(N2C(=O)c3ccc(Br)cc3C2=O)C(=O)N1] |
| 299 | 6 | 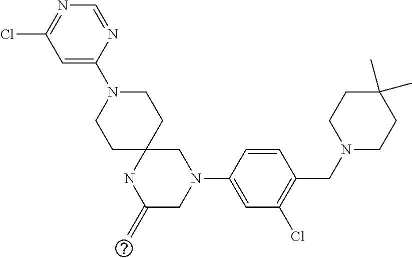 [SMILES: C=C1CN(c2ccc(CN3CCC(C)(C)CC3)c(Cl)c2)CC2(CCN(c3cc(Cl)ncn3)CC2)N1] | 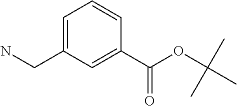 |
| | | 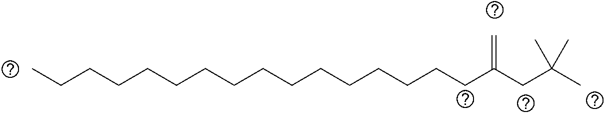 [SMILES: C=C(CCCCCCCCCCCCCCCC)CC(C)(C)C] | 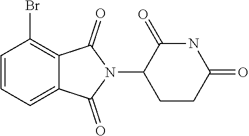 [SMILES: O=C1CCC(N2C(=O)c3cccc(Br)c3C2=O)C(=O)N1] |
| 300 | 6 | 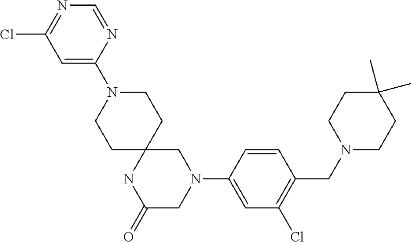 [SMILES: CC1(C)CCN(Cc2ccc(N3CC(=O)NC4(CCN(c5cc(Cl)ncn5)CC4)C3)cc2Cl)CC1] | 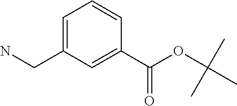 |
| | | 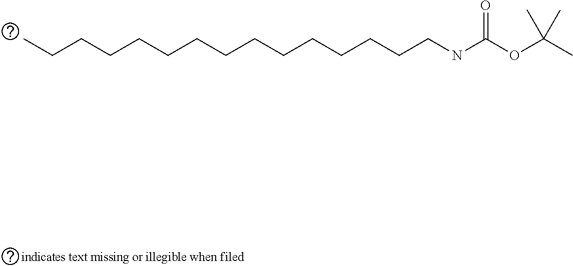 [SMILES: CCCCCCCCCCCCCCCNC(=O)OC(C)(C)C] | 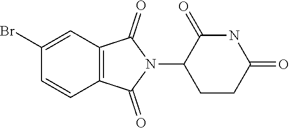 [SMILES: O=C1CCC(N2C(=O)c3ccc(Br)cc3C2=O)C(=O)N1] |
| 301 | 6 | 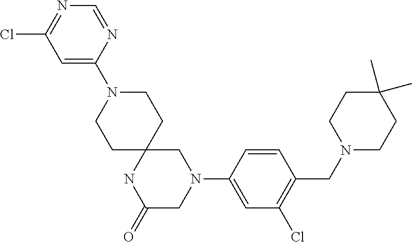 [SMILES: CC1(C)CCN(Cc2ccc(N3CC(=O)NC4(CCN(c5cc(Cl)ncn5)CC4)C3)cc2Cl)CC1] | 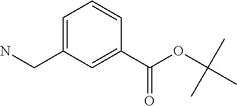 |
| | | 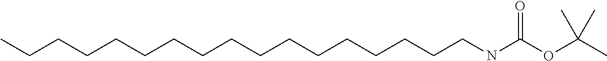 [SMILES: CCCCCCCCCCCCCCCCCNC(=O)OC(C)(C)C] | 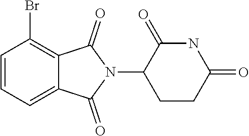 [SMILES: O=C1CCC(N2C(=O)c3cccc(Br)c3C2=O)C(=O)N1] |
| 302 | 6 | 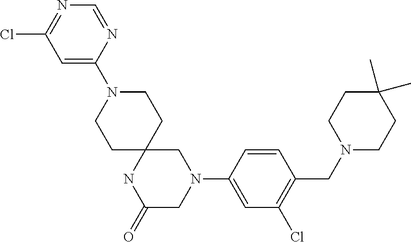 [SMILES: CC1(C)CCN(Cc2ccc(N3CC(=O)NC4(CCN(c5cc(Cl)ncn5)CC4)C3)cc2Cl)CC1] | 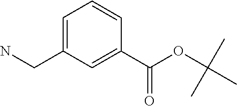 |
| | | 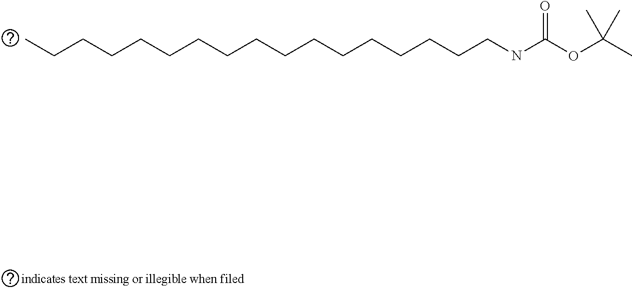 [SMILES: CCCCCCCCCCCCCCCCCNC(=O)OC(C)(C)C] | 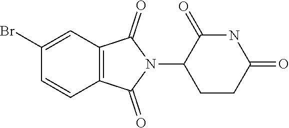 [SMILES: O=C1CCC(N2C(=O)c3ccc(Br)cc3C2=O)C(=O)N1] |
| 303 | 6 | 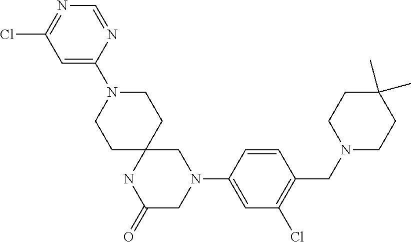 [SMILES: CC1(C)CCN(Cc2ccc(N3CC(=O)NC4(CCN(c5cc(Cl)ncn5)CC4)C3)cc2Cl)CC1] | 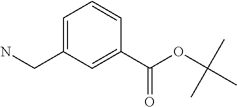 |
| | | 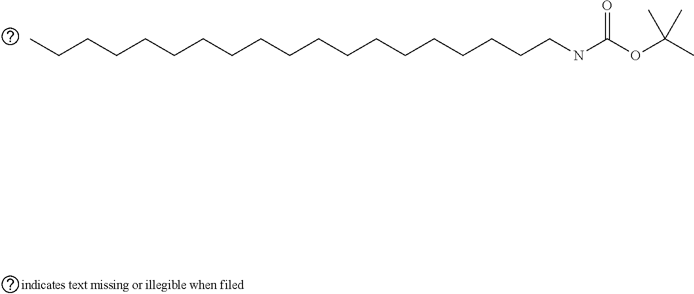 [SMILES: CCCCCCCCCCCCCCCCCCCNC(=O)OC(C)(C)C] | 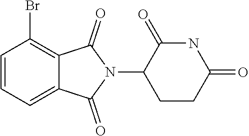 [SMILES: O=C1CCC(N2C(=O)c3cccc(Br)c3C2=O)C(=O)N1] |
| 304 | 6 | 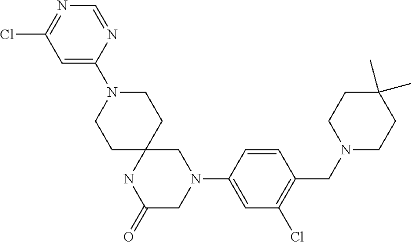 [SMILES: CC1(C)CCN(Cc2ccc(N3CC(=O)NC4(CCN(c5cc(Cl)ncn5)CC4)C3)cc2Cl)CC1] | 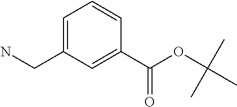 |
| | | 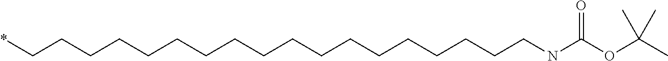 [SMILES: *CCCCCCCCCCCCCCCCCCNC(=O)OC(C)(C)C] | 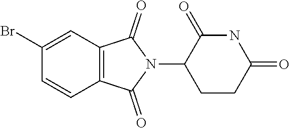 [SMILES: O=C1CCC(N2C(=O)c3ccc(Br)cc3C2=O)C(=O)N1] |
| 305 | 6 | 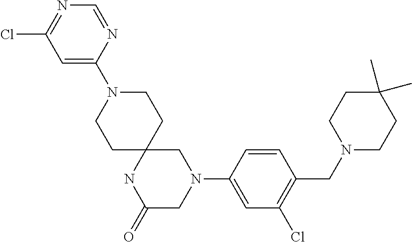 [SMILES: CC1(C)CCN(Cc2ccc(N3CC(=O)NC4(CCN(c5cc(Cl)ncn5)CC4)C3)cc2Cl)CC1] | 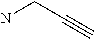 |
| | | 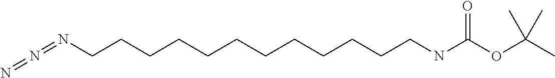 | 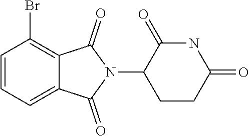 [SMILES: O=C1CCC(N2C(=O)c3cccc(Br)c3C2=O)C(=O)N1] |
| 306 | 6 | 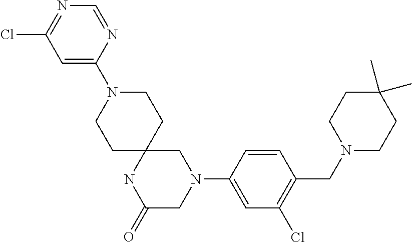 [SMILES: CC1(C)CCN(Cc2ccc(N3CC(=O)NC4(CCN(c5cc(Cl)ncn5)CC4)C3)cc2Cl)CC1] | 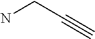 |
| | | 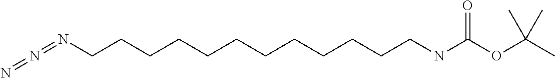 | 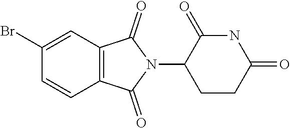 [SMILES: O=C1CCC(N2C(=O)c3ccc(Br)cc3C2=O)C(=O)N1] |
| 307 | 6 | 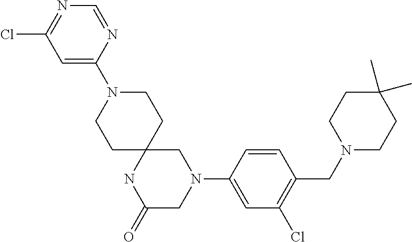 [SMILES: CC1(C)CCN(Cc2ccc(N3CC(=O)NC4(CCN(c5cc(Cl)ncn5)CC4)C3)cc2Cl)CC1] | 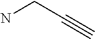 |
| | | 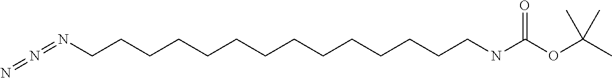 | 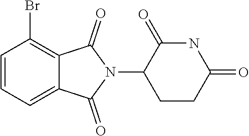 [SMILES: O=C1CCC(N2C(=O)c3cccc(Br)c3C2=O)C(=O)N1] |
| 308 | 6 | 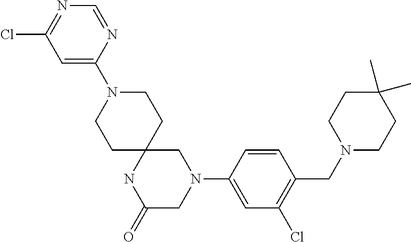 [SMILES: CC1(C)CCN(Cc2ccc(N3CC(=O)NC4(CCN(c5cc(Cl)ncn5)CC4)C3)cc2Cl)CC1] | 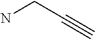 |
| | | 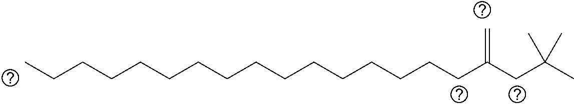 [SMILES: C=C(CCCCCCCCCCCCCCCC)CC(C)(C)C] | 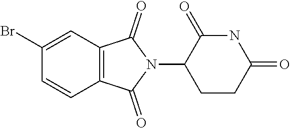 [SMILES: O=C1CCC(N2C(=O)c3ccc(Br)cc3C2=O)C(=O)N1] |
| 308 | 6 | 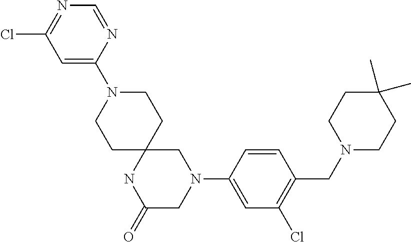 [SMILES: CC1(C)CCN(Cc2ccc(N3CC(=O)NC4(CCN(c5cc(Cl)ncn5)CC4)C3)cc2Cl)CC1] | 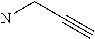 |
| | | 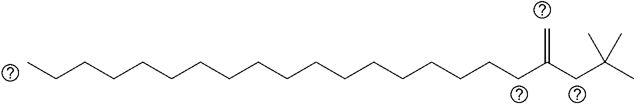 [SMILES: C=C(CCCCCCCCCCCCCCCCCC)CC(C)(C)C] | 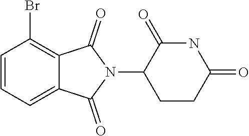 [SMILES: O=C1CCC(N2C(=O)c3cccc(Br)c3C2=O)C(=O)N1] |
| 310 | 6 | 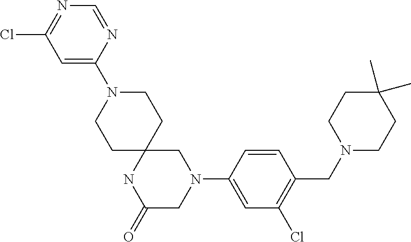 [SMILES: CC1(C)CCN(Cc2ccc(N3CC(=O)NC4(CCN(c5cc(Cl)ncn5)CC4)C3)cc2Cl)CC1] | 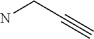 |
| | | 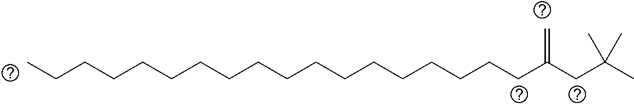 [SMILES: C=C(CCCCCCCCCCCCCCCCCC)CC(C)(C)C] | 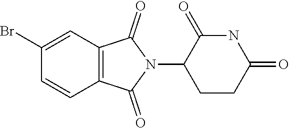 [SMILES: O=C1CCC(N2C(=O)c3ccc(Br)cc3C2=O)C(=O)N1] |
| 311 | 6 | 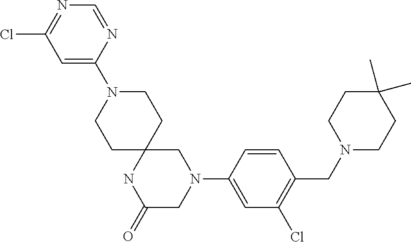 [SMILES: CC1(C)CCN(Cc2ccc(N3CC(=O)NC4(CCN(c5cc(Cl)ncn5)CC4)C3)cc2Cl)CC1] | 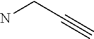 |
| | | 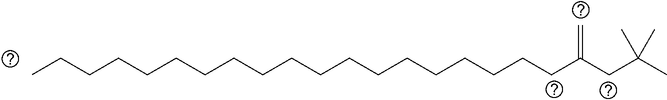 [SMILES: C=C(CCCCCCCCCCCCCCCCCCC)CC(C)(C)C] | 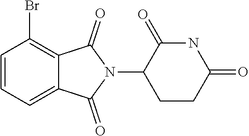 [SMILES: O=C1CCC(N2C(=O)c3cccc(Br)c3C2=O)C(=O)N1] |
| 312 | 6 | 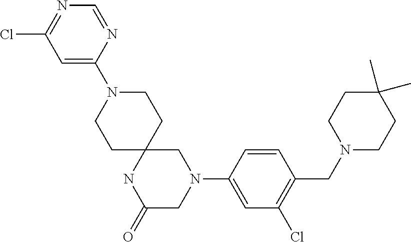 [SMILES: CC1(C)CCN(Cc2ccc(N3CC(=O)NC4(CCN(c5cc(Cl)ncn5)CC4)C3)cc2Cl)CC1] | 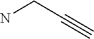 |
| | | 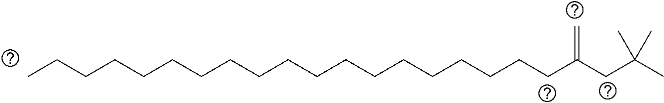 [SMILES: C=C(CCCCCCCCCCCCCCCCCCC)CC(C)(C)C] | 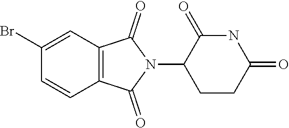 [SMILES: O=C1CCC(N2C(=O)c3ccc(Br)cc3C2=O)C(=O)N1] |
| 313 | 7 | 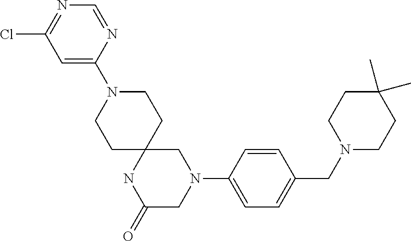 [SMILES: CC1(C)CCN(Cc2ccc(N3CC(=O)NC4(CCN(c5cc(Cl)ncn5)CC4)C3)cc2)CC1] | 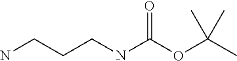 |
| | | 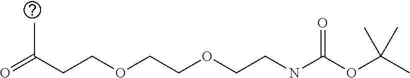 [SMILES: CC(=O)CCOCCOCCNC(=O)OC(C)(C)C] | 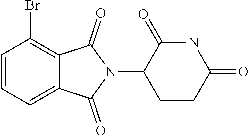 [SMILES: O=C1CCC(N2C(=O)c3cccc(Br)c3C2=O)C(=O)N1] |
| 314 | 2 | 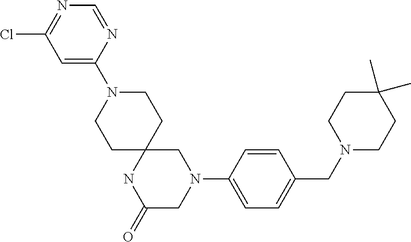 [SMILES: CC1(C)CCN(Cc2ccc(N3CC(=O)NC4(CCN(c5cc(Cl)ncn5)CC4)C3)cc2)CC1] | 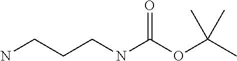 |
| | | 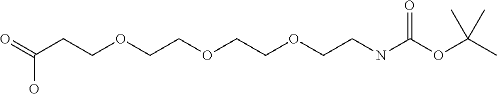 [SMILES: CC(C)(C)OC(=O)NCCOCCOCCOCCC(=O)O] | 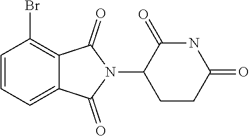 [SMILES: O=C1CCC(N2C(=O)c3cccc(Br)c3C2=O)C(=O)N1] |
| 315 | 7 | 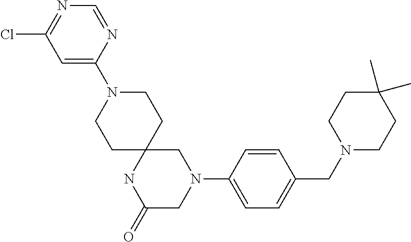 [SMILES: CC1(C)CCN(Cc2ccc(N3CC(=O)NC4(CCN(c5cc(Cl)ncn5)CC4)C3)cc2)CC1] | 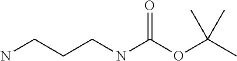 |
| | | 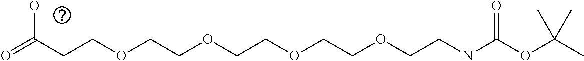 [SMILES: CC(C)(C)OC(=O)NCCOCCOCCOCCOCCC(=O)O] | 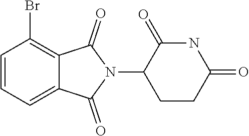 [SMILES: O=C1CCC(N2C(=O)c3cccc(Br)c3C2=O)C(=O)N1] |
| 316 | 7 | 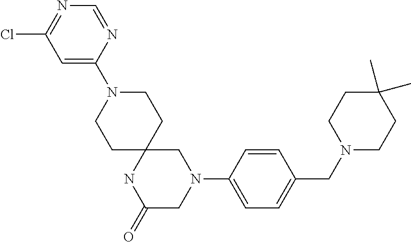 [SMILES: CC1(C)CCN(Cc2ccc(N3CC(=O)NC4(CCN(c5cc(Cl)ncn5)CC4)C3)cc2)CC1] | 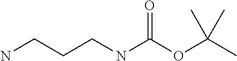 |
| | | 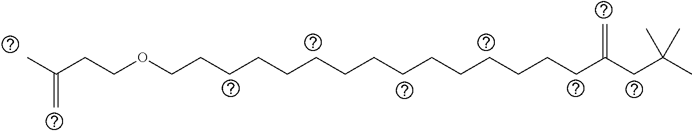 [SMILES: C=C(C)CCOCCCCCCCCCCCCCCCC(=C)CC(C)(C)C] | 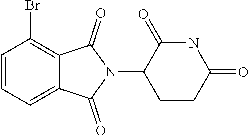 [SMILES: O=C1CCC(N2C(=O)c3cccc(Br)c3C2=O)C(=O)N1] |
| 317 | 7 | 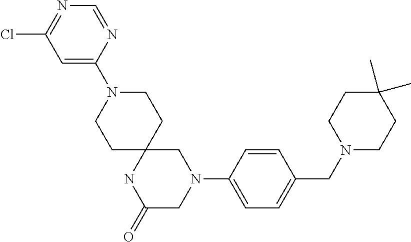 [SMILES: CC1(C)CCN(Cc2ccc(N3CC(=O)NC4(CCN(c5cc(Cl)ncn5)CC4)C3)cc2)CC1] | 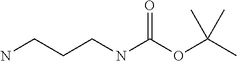 |
| | | 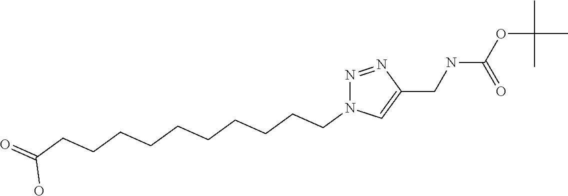 [SMILES: CC(C)(C)OC(=O)NCc1cn(CCCCCCCCCCC(=O)O)nn1] | 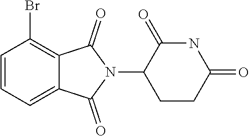 [SMILES: O=C1CCC(N2C(=O)c3cccc(Br)c3C2=O)C(=O)N1] |
| 318 | 7 | 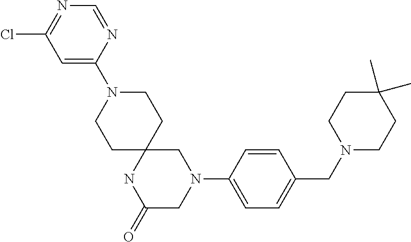 [SMILES: CC1(C)CCN(Cc2ccc(N3CC(=O)NC4(CCN(c5cc(Cl)ncn5)CC4)C3)cc2)CC1] | 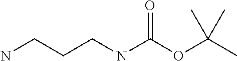 |
| | | 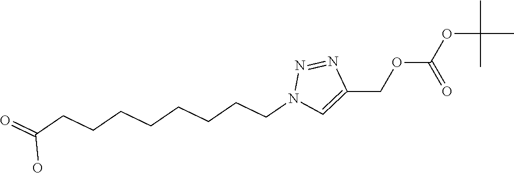 [SMILES: CC(C)(C)OC(=O)OCc1cn(CCCCCCCCC(=O)O)nn1] | 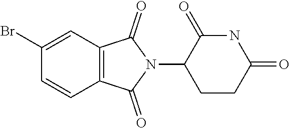 [SMILES: O=C1CCC(N2C(=O)c3ccc(Br)cc3C2=O)C(=O)N1] |
| 319 | 7 | 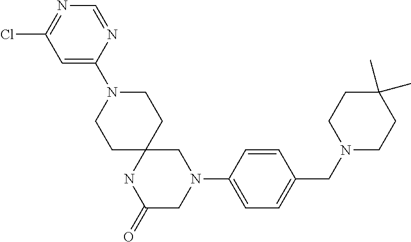 [SMILES: CC1(C)CCN(Cc2ccc(N3CC(=O)NC4(CCN(c5cc(Cl)ncn5)CC4)C3)cc2)CC1] | 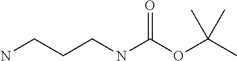 |
| | | 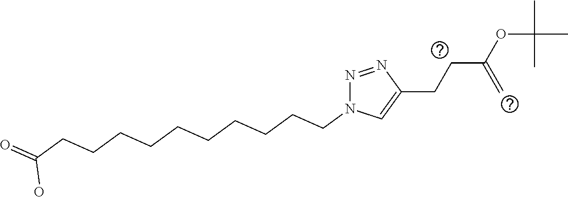 [SMILES: C=C(CCc1cn(CCCCCCCCCCC(=O)O)nn1)OC(C)(C)C] | 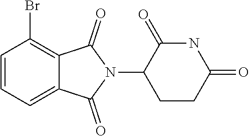 [SMILES: O=C1CCC(N2C(=O)c3cccc(Br)c3C2=O)C(=O)N1] |
| 320 | 7 | 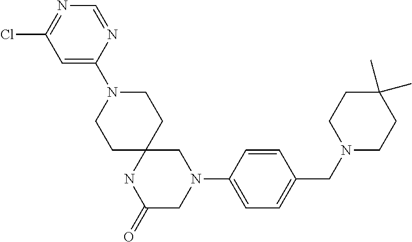 [SMILES: CC1(C)CCN(Cc2ccc(N3CC(=O)NC4(CCN(c5cc(Cl)ncn5)CC4)C3)cc2)CC1] | 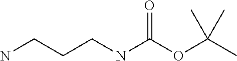 |
| | | 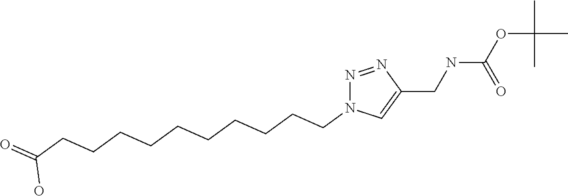 [SMILES: CC(C)(C)OC(=O)NCc1cn(CCCCCCCCCCC(=O)O)nn1] | 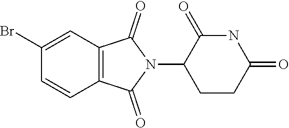 [SMILES: O=C1CCC(N2C(=O)c3ccc(Br)cc3C2=O)C(=O)N1] |
| 321 | 7 | 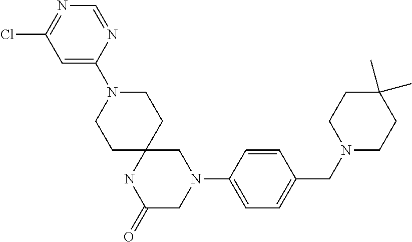 [SMILES: CC1(C)CCN(Cc2ccc(N3CC(=O)NC4(CCN(c5cc(Cl)ncn5)CC4)C3)cc2)CC1] | 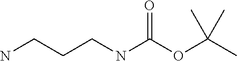 |
| | | 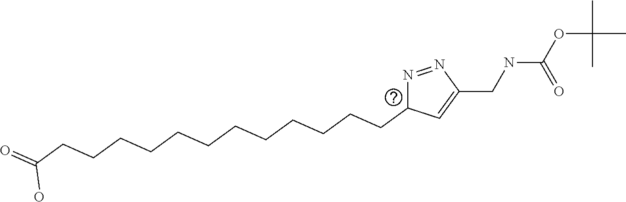 [SMILES: CC(C)(C)OC(=O)NCC1=CC(CCCCCCCCCCCCC(=O)O)N=N1] | 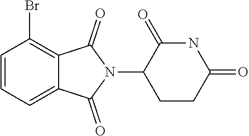 [SMILES: O=C1CCC(N2C(=O)c3cccc(Br)c3C2=O)C(=O)N1] |
| 322 | 7 | 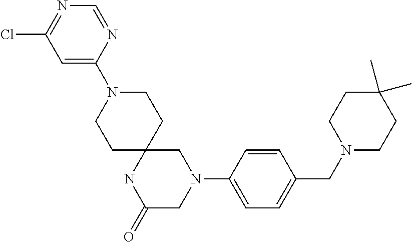 [SMILES: CC1(C)CCN(Cc2ccc(N3CC(=O)NC4(CCN(c5cc(Cl)ncn5)CC4)C3)cc2)CC1] | 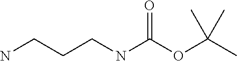 |
| | | 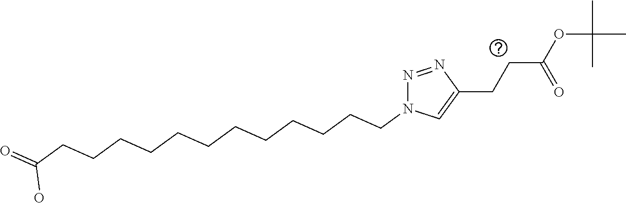 [SMILES: CC(C)(C)OC(=O)CCc1cn(CCCCCCCCCCCCC(=O)O)nn1] | 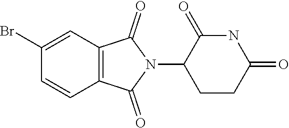 [SMILES: O=C1CCC(N2C(=O)c3ccc(Br)cc3C2=O)C(=O)N1] |
| 323 | 7 | 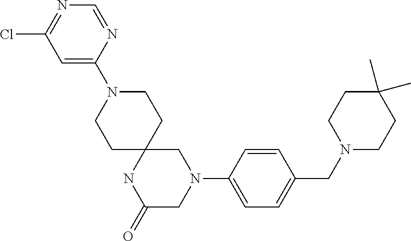 [SMILES: CC1(C)CCN(Cc2ccc(N3CC(=O)NC4(CCN(c5cc(Cl)ncn5)CC4)C3)cc2)CC1] | 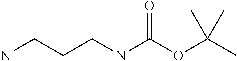 |
| | | 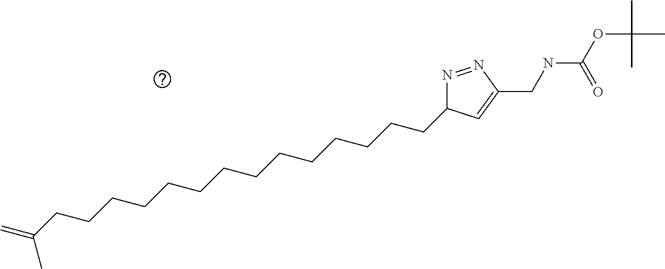 [SMILES: C=C(C)CCCCCCCCCCCCCCC1C=C(CNC(=O)OC(C)(C)C)N=N1] | 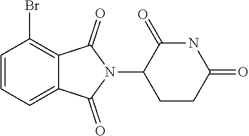 [SMILES: O=C1CCC(N2C(=O)c3cccc(Br)c3C2=O)C(=O)N1] |
| 324 | 7 | 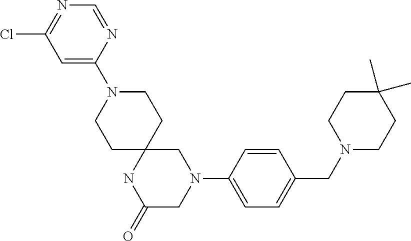 [SMILES: CC1(C)CCN(Cc2ccc(N3CC(=O)NC4(CCN(c5cc(Cl)ncn5)CC4)C3)cc2)CC1] | 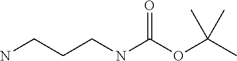 |
| | | 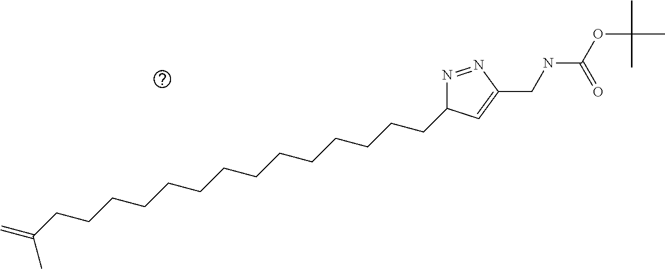 [SMILES: C=C(C)CCCCCCCCCCCCCCC1C=C(CNC(=O)OC(C)(C)C)N=N1] | 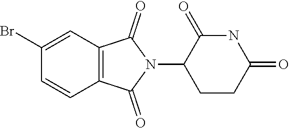 [SMILES: O=C1CCC(N2C(=O)c3ccc(Br)cc3C2=O)C(=O)N1] |
| 325 | 7 | 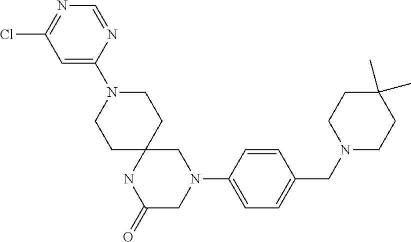 [SMILES: CC1(C)CCN(Cc2ccc(N3CC(=O)NC4(CCN(c5cc(Cl)ncn5)CC4)C3)cc2)CC1] | 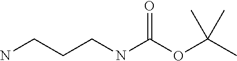 |
| | | 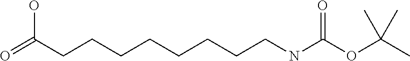 [SMILES: CC(C)(C)OC(=O)NCCCCCCCCC(=O)O] | 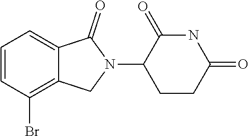 [SMILES: O=C1CCC(N2Cc3c(Br)cccc3C2=O)C(=O)N1] |
| 326 | 7 | 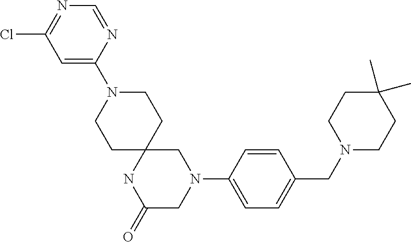 [SMILES: CC1(C)CCN(Cc2ccc(N3CC(=O)NC4(CCN(c5cc(Cl)ncn5)CC4)C3)cc2)CC1] | 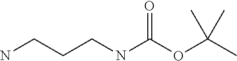 |
| | | 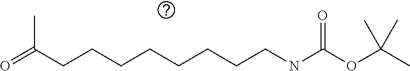 [SMILES: CC(=O)CCCCCCCCNC(=O)OC(C)(C)C] | 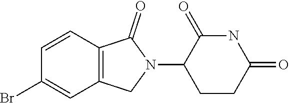 [SMILES: O=C1CCC(N2Cc3cc(Br)ccc3C2=O)C(=O)N1] |
| 327 | 7 | 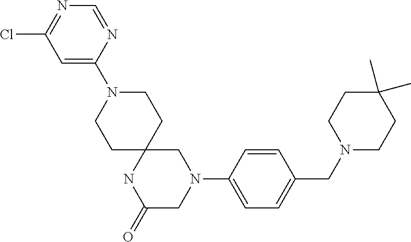 [SMILES: CC1(C)CCN(Cc2ccc(N3CC(=O)NC4(CCN(c5cc(Cl)ncn5)CC4)C3)cc2)CC1] | 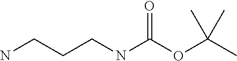 |
| | | 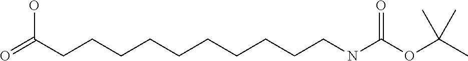 [SMILES: CC(C)(C)OC(=O)NCCCCCCCCCCC(=O)O] | 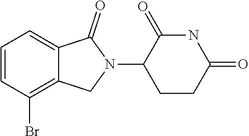 [SMILES: O=C1CCC(N2Cc3c(Br)cccc3C2=O)C(=O)N1] |
| 328 | 7 | 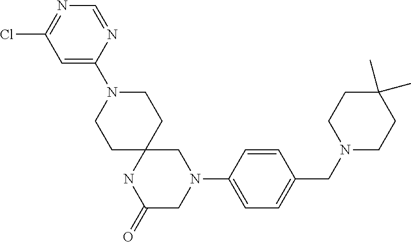 [SMILES: CC1(C)CCN(Cc2ccc(N3CC(=O)NC4(CCN(c5cc(Cl)ncn5)CC4)C3)cc2)CC1] | 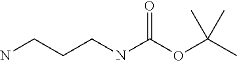 |
| | | 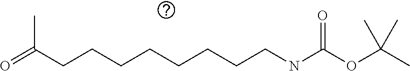 [SMILES: CC(=O)CCCCCCCCNC(=O)OC(C)(C)C] | 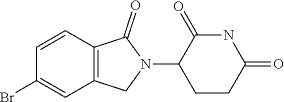 [SMILES: O=C1CCC(N2Cc3cc(Br)ccc3C2=O)C(=O)N1] |
| 329 | 7 | 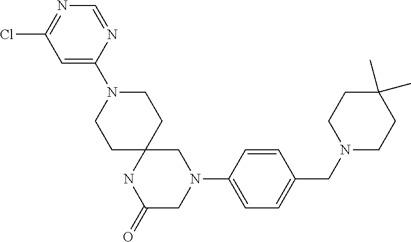 [SMILES: CC1(C)CCN(Cc2ccc(N3CC(=O)NC4(CCN(c5cc(Cl)ncn5)CC4)C3)cc2)CC1] | 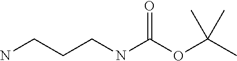 |
| | | 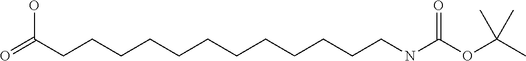 [SMILES: CC(C)(C)OC(=O)NCCCCCCCCCCCCC(=O)O] | 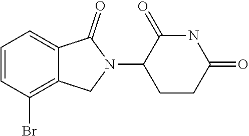 [SMILES: O=C1CCC(N2Cc3c(Br)cccc3C2=O)C(=O)N1] |
| 330 | 7 | 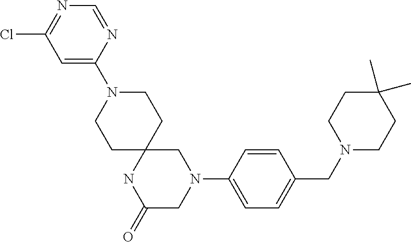 [SMILES: CC1(C)CCN(Cc2ccc(N3CC(=O)NC4(CCN(c5cc(Cl)ncn5)CC4)C3)cc2)CC1] | 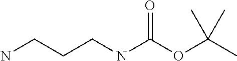 |
| | | 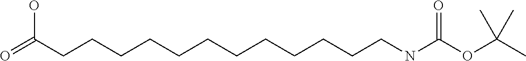 [SMILES: CC(C)(C)OC(=O)NCCCCCCCCCCCCC(=O)O] | 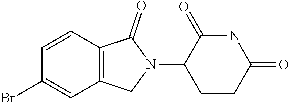 [SMILES: O=C1CCC(N2Cc3cc(Br)ccc3C2=O)C(=O)N1] |
| 321 | 7 | 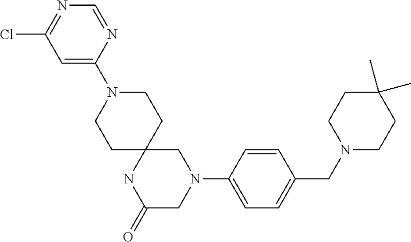 [SMILES: CC1(C)CCN(Cc2ccc(N3CC(=O)NC4(CCN(c5cc(Cl)ncn5)CC4)C3)cc2)CC1] | 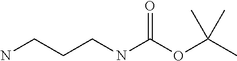 |
| | | 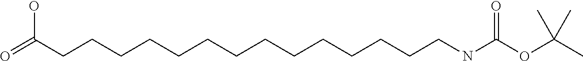 [SMILES: CC(C)(C)OC(=O)NCCCCCCCCCCCCCCC(=O)O] | 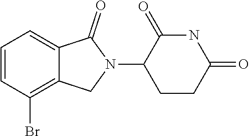 [SMILES: O=C1CCC(N2Cc3c(Br)cccc3C2=O)C(=O)N1] |
| 322 | 7 | 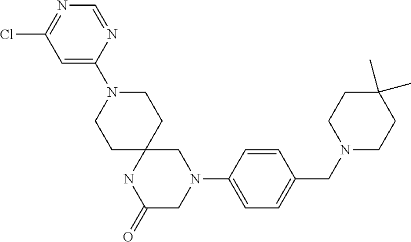 [SMILES: CC1(C)CCN(Cc2ccc(N3CC(=O)NC4(CCN(c5cc(Cl)ncn5)CC4)C3)cc2)CC1] | 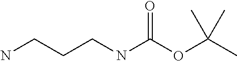 |
| | | 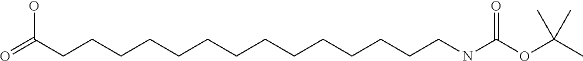 [SMILES: CC(C)(C)OC(=O)NCCCCCCCCCCCCCCC(=O)O] | 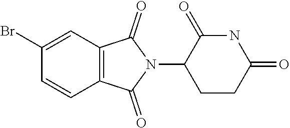 [SMILES: O=C1CCC(N2C(=O)c3ccc(Br)cc3C2=O)C(=O)N1] |
| 353 | 7 | 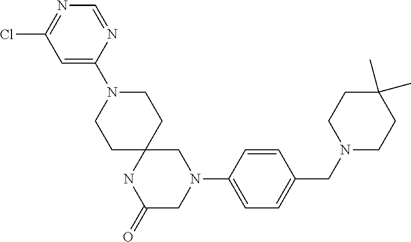 [SMILES: CC1(C)CCN(Cc2ccc(N3CC(=O)NC4(CCN(c5cc(Cl)ncn5)CC4)C3)cc2)CC1] | 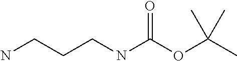 |
| | | 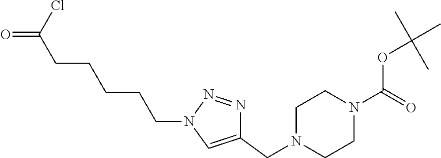 [SMILES: CC(C)(C)OC(=O)N1CCN(Cc2cn(CCCCCC(=O)Cl)nn2)CC1] | 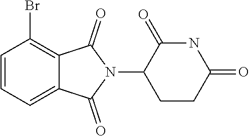 [SMILES: O=C1CCC(N2C(=O)c3cccc(Br)c3C2=O)C(=O)N1] |
| 354 | 7 | 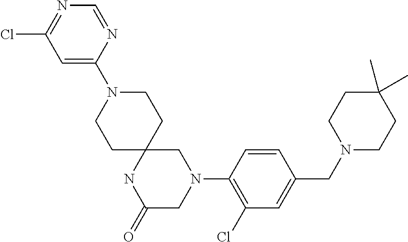 [SMILES: CC1(C)CCN(Cc2ccc(N3CC(=O)NC4(CCN(c5cc(Cl)ncn5)CC4)C3)c(Cl)c2)CC1] | 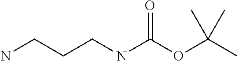 |
| | | 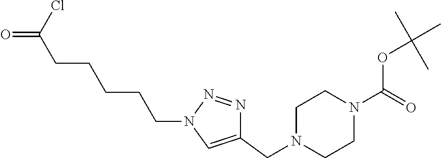 [SMILES: CC(C)(C)OC(=O)N1CCN(Cc2cn(CCCCCC(=O)Cl)nn2)CC1] | 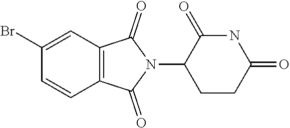 [SMILES: O=C1CCC(N2C(=O)c3ccc(Br)cc3C2=O)C(=O)N1] |
| 355 | 7 | 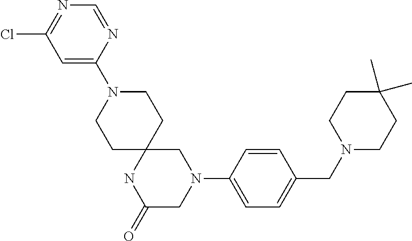 [SMILES: CC1(C)CCN(Cc2ccc(N3CC(=O)NC4(CCN(c5cc(Cl)ncn5)CC4)C3)cc2)CC1] | 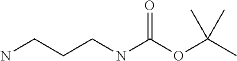 |
| | | 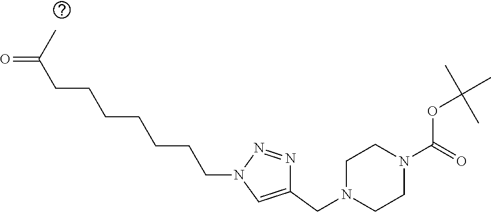 [SMILES: CC(=O)CCCCCCCn1cc(CN2CCN(C(=O)OC(C)(C)C)CC2)nn1] | 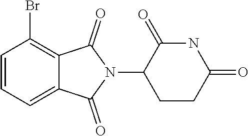 [SMILES: O=C1CCC(N2C(=O)c3cccc(Br)c3C2=O)C(=O)N1] |
| 536 | 7 | 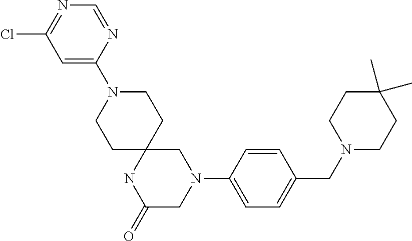 [SMILES: CC1(C)CCN(Cc2ccc(N3CC(=O)NC4(CCN(c5cc(Cl)ncn5)CC4)C3)cc2)CC1] | 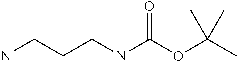 |
| | | 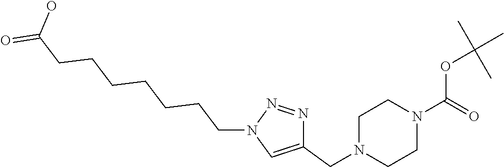 [SMILES: CC(C)(C)OC(=O)N1CCN(Cc2cn(CCCCCCCC(=O)O)nn2)CC1] | 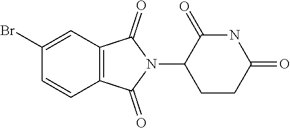 [SMILES: O=C1CCC(N2C(=O)c3ccc(Br)cc3C2=O)C(=O)N1] |
| 337 | 7 | 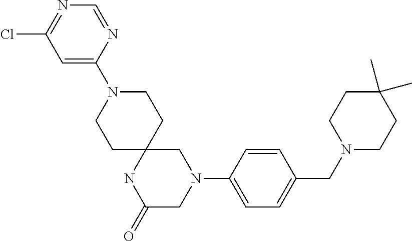 [SMILES: CC1(C)CCN(Cc2ccc(N3CC(=O)NC4(CCN(c5cc(Cl)ncn5)CC4)C3)cc2)CC1] | 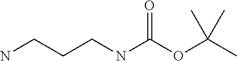 |
| | | 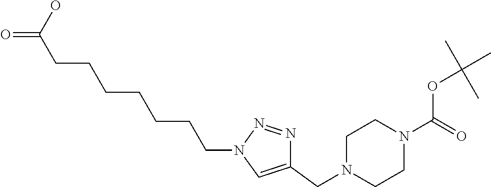 [SMILES: CC(C)(C)OC(=O)N1CCN(Cc2cn(CCCCCCCC(=O)O)nn2)CC1] | 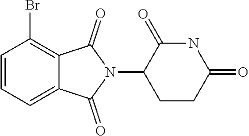 [SMILES: O=C1CCC(N2C(=O)c3cccc(Br)c3C2=O)C(=O)N1] |
| 338 | 7 | 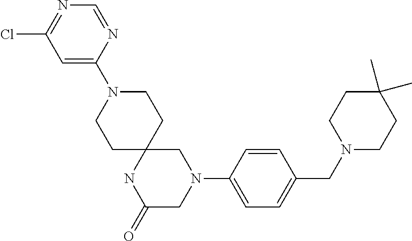 [SMILES: CC1(C)CCN(Cc2ccc(N3CC(=O)NC4(CCN(c5cc(Cl)ncn5)CC4)C3)cc2)CC1] | 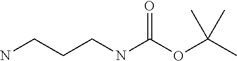 |
| | | 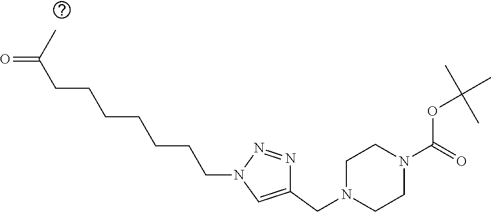 [SMILES: CC(=O)CCCCCCCn1cc(CN2CCN(C(=O)OC(C)(C)C)CC2)nn1] | 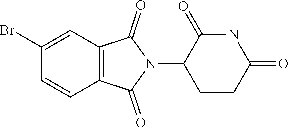 [SMILES: O=C1CCC(N2C(=O)c3ccc(Br)cc3C2=O)C(=O)N1] |
| 339 | 7 | 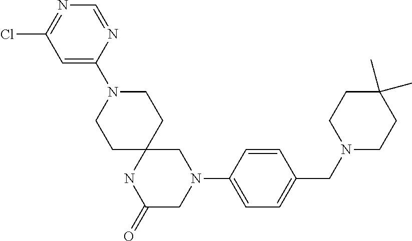 [SMILES: CC1(C)CCN(Cc2ccc(N3CC(=O)NC4(CCN(c5cc(Cl)ncn5)CC4)C3)cc2)CC1] | 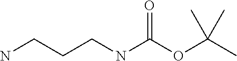 |
| | | 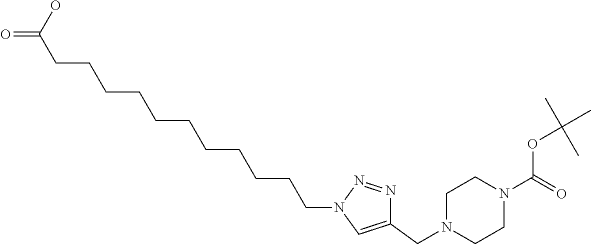 [SMILES: CC(C)(C)OC(=O)N1CCN(Cc2cn(CCCCCCCCCCCC(=O)O)nn2)CC1] | 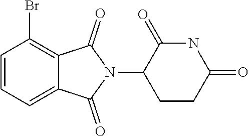 [SMILES: O=C1CCC(N2C(=O)c3cccc(Br)c3C2=O)C(=O)N1] |
| 340 | 7 | 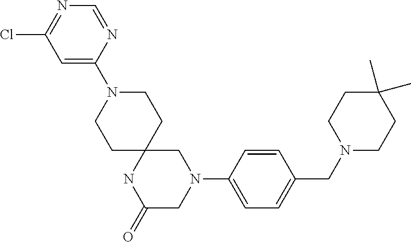 [SMILES: CC1(C)CCN(Cc2ccc(N3CC(=O)NC4(CCN(c5cc(Cl)ncn5)CC4)C3)cc2)CC1] | 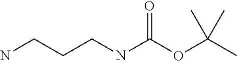 |
| | | 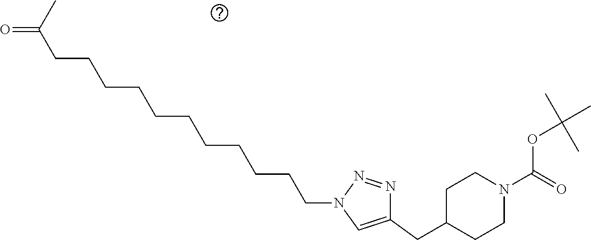 [SMILES: CC(=O)CCCCCCCCCCCn1cc(CC2CCN(C(=O)OC(C)(C)C)CC2)nn1] | 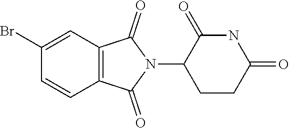 [SMILES: O=C1CCC(N2C(=O)c3ccc(Br)cc3C2=O)C(=O)N1] |
| 341 | 7 | 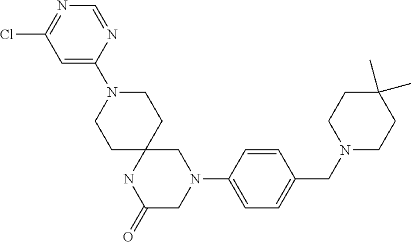 [SMILES: CC1(C)CCN(Cc2ccc(N3CC(=O)NC4(CCN(c5cc(Cl)ncn5)CC4)C3)cc2)CC1] | 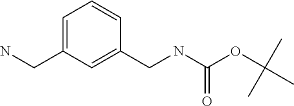 [SMILES: CC(C)(C)OC(=O)NCc1cccc(CN)c1] |
| | | 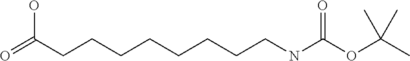 [SMILES: CC(C)(C)OC(=O)NCCCCCCCCC(=O)O] | 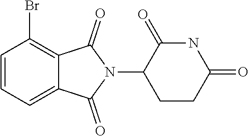 [SMILES: O=C1CCC(N2C(=O)c3cccc(Br)c3C2=O)C(=O)N1] |
| 342 | 7 | 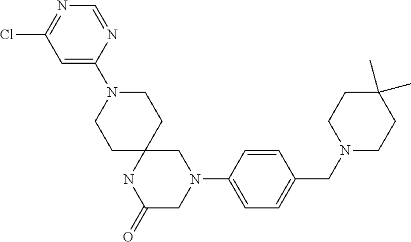 [SMILES: CC1(C)CCN(Cc2ccc(N3CC(=O)NC4(CCN(c5cc(Cl)ncn5)CC4)C3)cc2)CC1] | 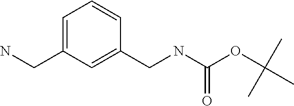 [SMILES: CC(C)(C)OC(=O)NCc1cccc(CN)c1] |
| | | 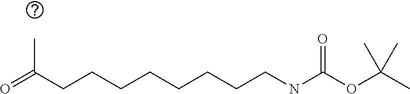 [SMILES: CC(=O)CCCCCCCCNC(=O)OC(C)(C)C] | 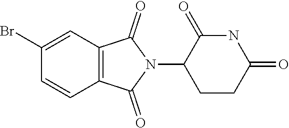 [SMILES: O=C1CCC(N2C(=O)c3ccc(Br)cc3C2=O)C(=O)N1] |
| 343 | 7 | 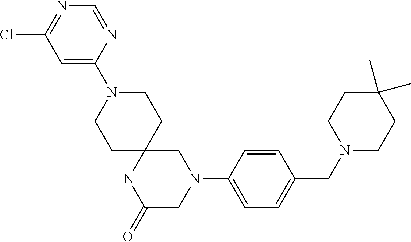 [SMILES: CC1(C)CCN(Cc2ccc(N3CC(=O)NC4(CCN(c5cc(Cl)ncn5)CC4)C3)cc2)CC1] | 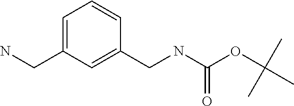 [SMILES: CC(C)(C)OC(=O)NCc1cccc(CN)c1] |
| | | 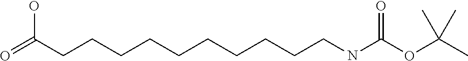 [SMILES: CC(C)(C)OC(=O)NCCCCCCCCCCC(=O)O] | 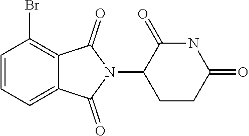 [SMILES: O=C1CCC(N2C(=O)c3cccc(Br)c3C2=O)C(=O)N1] |
| 344 | 7 | 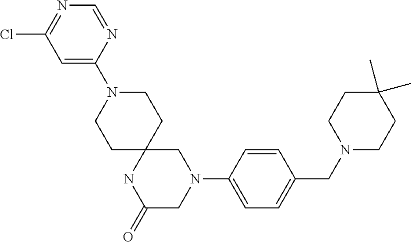 [SMILES: CC1(C)CCN(Cc2ccc(N3CC(=O)NC4(CCN(c5cc(Cl)ncn5)CC4)C3)cc2)CC1] | 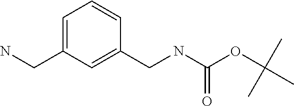 [SMILES: CC(C)(C)OC(=O)NCc1cccc(CN)c1] |
| | | 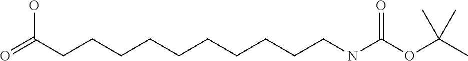 [SMILES: CC(C)(C)OC(=O)NCCCCCCCCCCC(=O)O] | 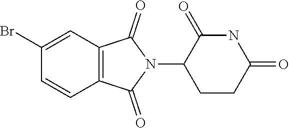 [SMILES: O=C1CCC(N2C(=O)c3ccc(Br)cc3C2=O)C(=O)N1] |
| 345 | 7 | 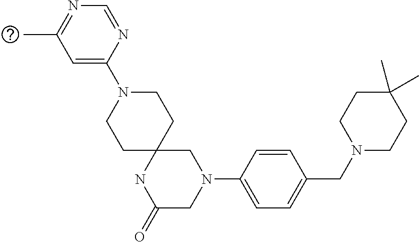 | 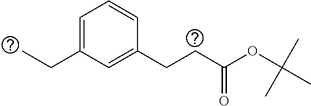 [SMILES: CCc1cccc(CCC(=O)OC(C)(C)C)c1] |
| | | 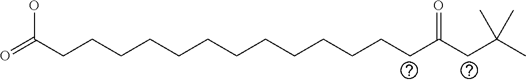 [SMILES: CC(C)(C)CC(=O)CCCCCCCCCCCCCC(=O)O] | 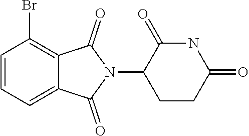 [SMILES: O=C1CCC(N2C(=O)c3cccc(Br)c3C2=O)C(=O)N1] |
| 346 | 7 | 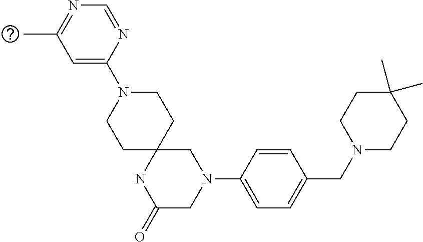 | 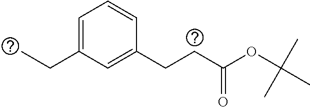 [SMILES: CCc1cccc(CCC(=O)OC(C)(C)C)c1] |
| | | 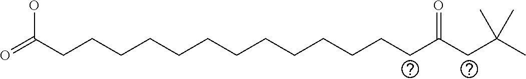 [SMILES: CC(C)(C)CC(=O)CCCCCCCCCCCCCC(=O)O] | 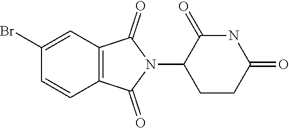 [SMILES: O=C1CCC(N2C(=O)c3ccc(Br)cc3C2=O)C(=O)N1] |
| 347 | 7 | 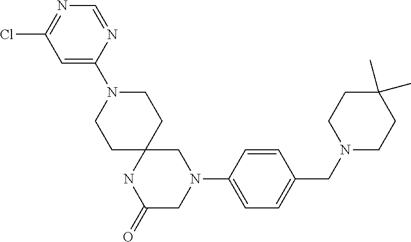 [SMILES: CC1(C)CCN(Cc2ccc(N3CC(=O)NC4(CCN(c5cc(Cl)ncn5)CC4)C3)cc2)CC1] | 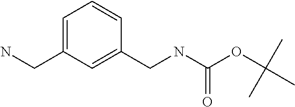 [SMILES: CC(C)(C)OC(=O)NCc1cccc(CN)c1] |
| | | 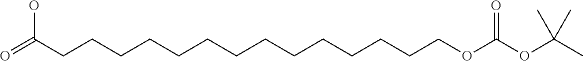 [SMILES: CC(C)(C)OC(=O)OCCCCCCCCCCCCCCC(=O)O] | 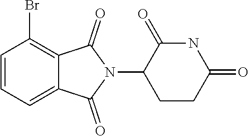 [SMILES: O=C1CCC(N2C(=O)c3cccc(Br)c3C2=O)C(=O)N1] |
| 348 | 7 | 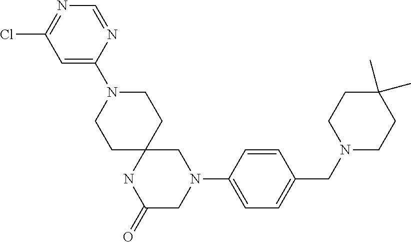 [SMILES: CC1(C)CCN(Cc2ccc(N3CC(=O)NC4(CCN(c5cc(Cl)ncn5)CC4)C3)cc2)CC1] | 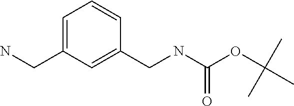 [SMILES: CC(C)(C)OC(=O)NCc1cccc(CN)c1] |
| | | 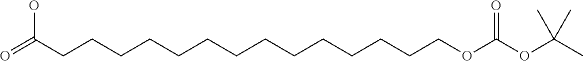 [SMILES: CC(C)(C)OC(=O)OCCCCCCCCCCCCCCC(=O)O] | 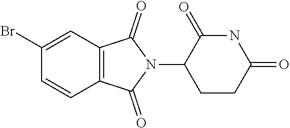 [SMILES: O=C1CCC(N2C(=O)c3ccc(Br)cc3C2=O)C(=O)N1] |
| 349 | 7 | 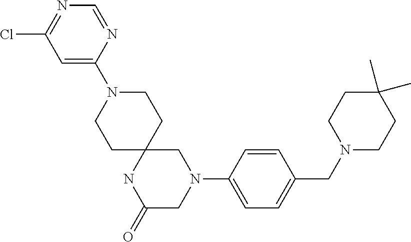 [SMILES: CC1(C)CCN(Cc2ccc(N3CC(=O)NC4(CCN(c5cc(Cl)ncn5)CC4)C3)cc2)CC1] | 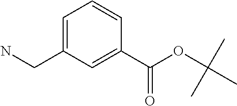 |
| | | 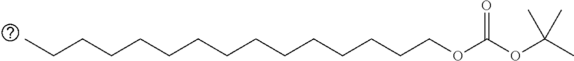 [SMILES: CCCCCCCCCCCCCCCOC(=O)OC(C)(C)C] | 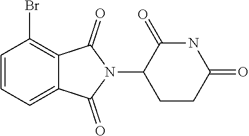 [SMILES: O=C1CCC(N2C(=O)c3cccc(Br)c3C2=O)C(=O)N1] |
| 350 | 7 | 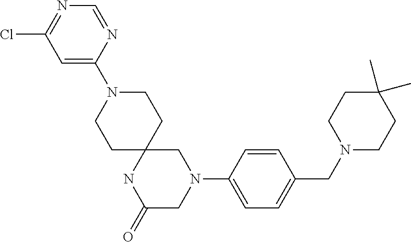 [SMILES: CC1(C)CCN(Cc2ccc(N3CC(=O)NC4(CCN(c5cc(Cl)ncn5)CC4)C3)cc2)CC1] | 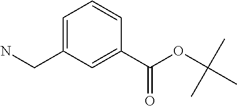 |
| | | 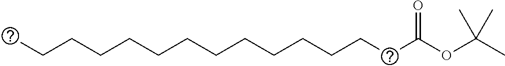 [SMILES: CCCCCCCCCCCCCCC(=O)OC(C)(C)C] | 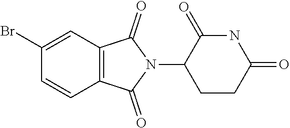 [SMILES: O=C1CCC(N2C(=O)c3ccc(Br)cc3C2=O)C(=O)N1] |
| 351 | 7 | 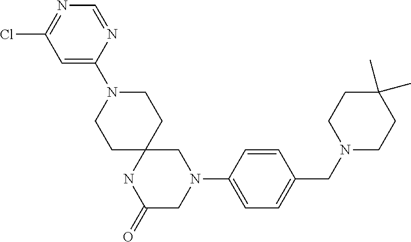 [SMILES: CC1(C)CCN(Cc2ccc(N3CC(=O)NC4(CCN(c5cc(Cl)ncn5)CC4)C3)cc2)CC1] | 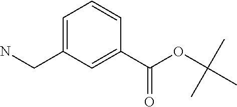 |
| | | 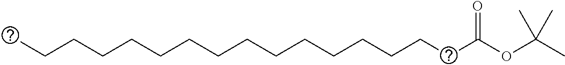 [SMILES: CCCCCCCCCCCCCCCCC(=O)OC(C)(C)C] | 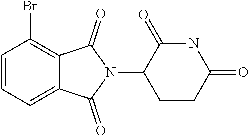 [SMILES: O=C1CCC(N2C(=O)c3cccc(Br)c3C2=O)C(=O)N1] |
| 352 | 7 | 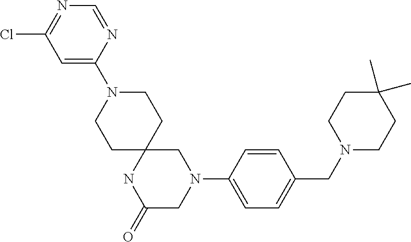 [SMILES: CC1(C)CCN(Cc2ccc(N3CC(=O)NC4(CCN(c5cc(Cl)ncn5)CC4)C3)cc2)CC1] | 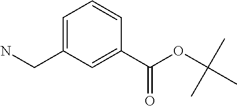 |
| | | 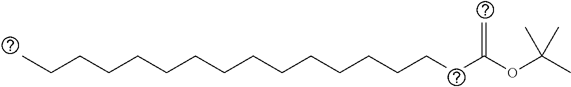 [SMILES: C=C(CCCCCCCCCCCCCCCC)OC(C)(C)C] | 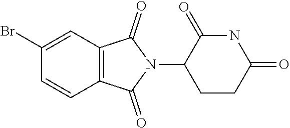 [SMILES: O=C1CCC(N2C(=O)c3ccc(Br)cc3C2=O)C(=O)N1] |
| 353 | 7 | 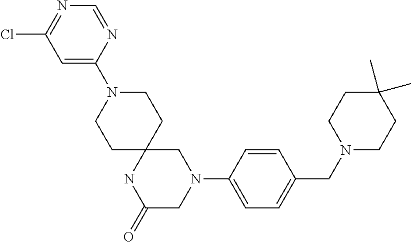 [SMILES: CC1(C)CCN(Cc2ccc(N3CC(=O)NC4(CCN(c5cc(Cl)ncn5)CC4)C3)cc2)CC1] | 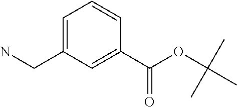 |
| | | 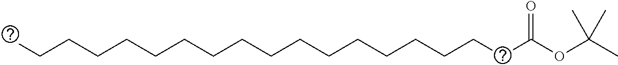 [SMILES: CCCCCCCCCCCCCCCCCCC(=O)OC(C)(C)C] | 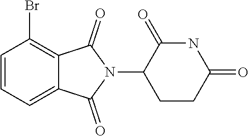 [SMILES: O=C1CCC(N2C(=O)c3cccc(Br)c3C2=O)C(=O)N1] |
| 354 | 7 | 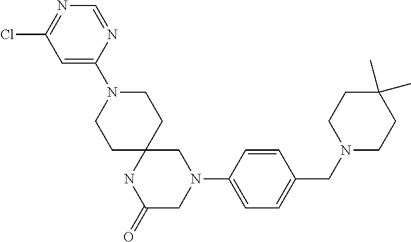 [SMILES: CC1(C)CCN(Cc2ccc(N3CC(=O)NC4(CCN(c5cc(Cl)ncn5)CC4)C3)cc2)CC1] | 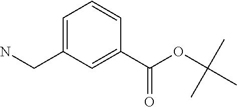 |
| | | 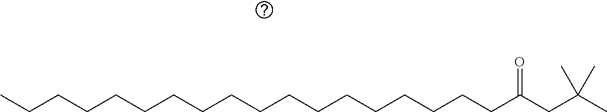 [SMILES: CCCCCCCCCCCCCCCCCCC(=O)CC(C)(C)C] | 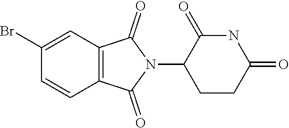 [SMILES: O=C1CCC(N2C(=O)c3ccc(Br)cc3C2=O)C(=O)N1] |
| 355 | 7 | 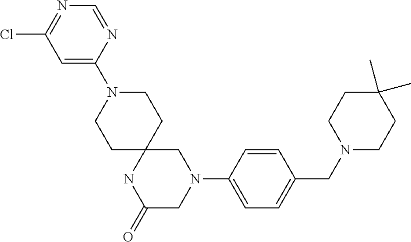 [SMILES: CC1(C)CCN(Cc2ccc(N3CC(=O)NC4(CCN(c5cc(Cl)ncn5)CC4)C3)cc2)CC1] | 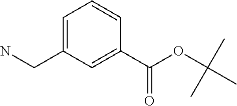 |
| | | 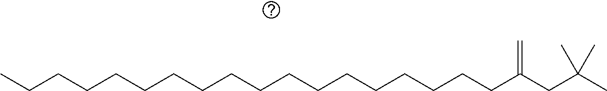 [SMILES: C=C(CCCCCCCCCCCCCCCCCC)CC(C)(C)C] | 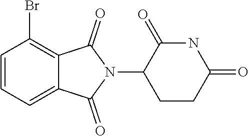 [SMILES: O=C1CCC(N2C(=O)c3cccc(Br)c3C2=O)C(=O)N1] |
| 356 | 7 | 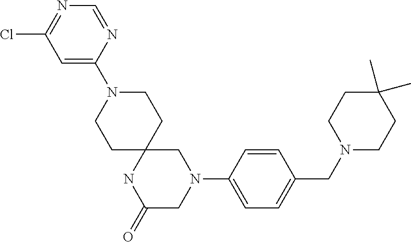 [SMILES: CC1(C)CCN(Cc2ccc(N3CC(=O)NC4(CCN(c5cc(Cl)ncn5)CC4)C3)cc2)CC1] | 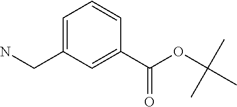 |
| | | 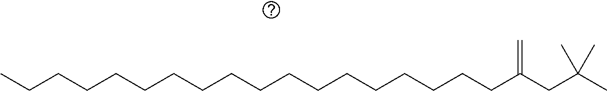 [SMILES: C=C(CCCCCCCCCCCCCCCCCC)CC(C)(C)C] | 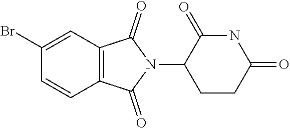 [SMILES: O=C1CCC(N2C(=O)c3ccc(Br)cc3C2=O)C(=O)N1] |
| 357 | 7 | 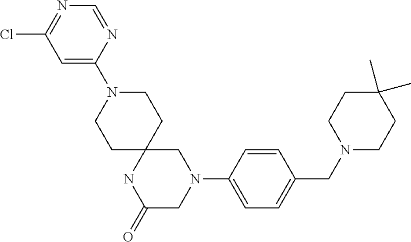 [SMILES: CC1(C)CCN(Cc2ccc(N3CC(=O)NC4(CCN(c5cc(Cl)ncn5)CC4)C3)cc2)CC1] | 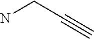 |
| | | 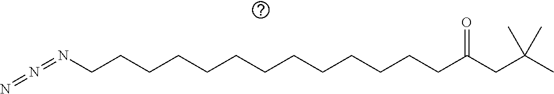 | 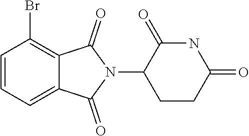 [SMILES: O=C1CCC(N2C(=O)c3cccc(Br)c3C2=O)C(=O)N1] |
| 358 | 7 | 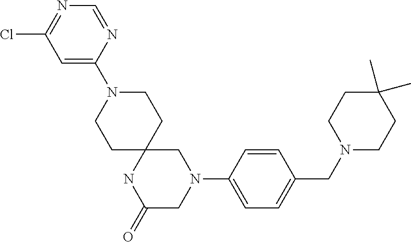 [SMILES: CC1(C)CCN(Cc2ccc(N3CC(=O)NC4(CCN(c5cc(Cl)ncn5)CC4)C3)cc2)CC1] | 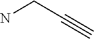 |
| | | 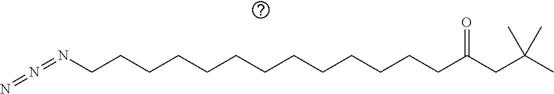 | 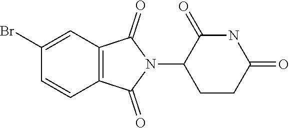 [SMILES: O=C1CCC(N2C(=O)c3ccc(Br)cc3C2=O)C(=O)N1] |
| 359 | 7 | 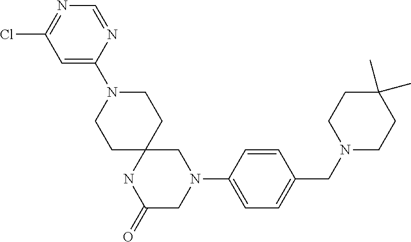 [SMILES: CC1(C)CCN(Cc2ccc(N3CC(=O)NC4(CCN(c5cc(Cl)ncn5)CC4)C3)cc2)CC1] | 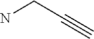 |
| | | 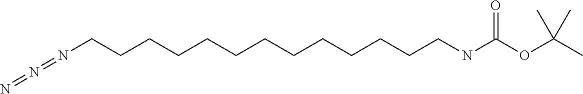 | 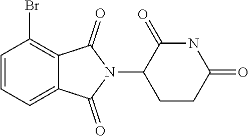 [SMILES: O=C1CCC(N2C(=O)c3cccc(Br)c3C2=O)C(=O)N1] |
| 360 | 7 | 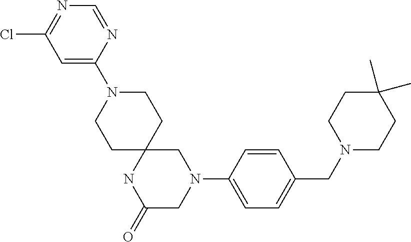 [SMILES: CC1(C)CCN(Cc2ccc(N3CC(=O)NC4(CCN(c5cc(Cl)ncn5)CC4)C3)cc2)CC1] | 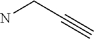 |
| | | 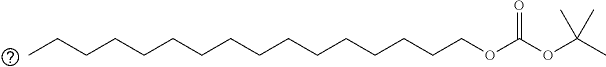 [SMILES: CCCCCCCCCCCCCCCCOC(=O)OC(C)(C)C] | 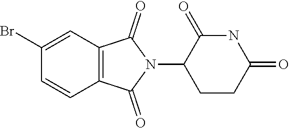 [SMILES: O=C1CCC(N2C(=O)c3ccc(Br)cc3C2=O)C(=O)N1] |
| 361 | 7 | 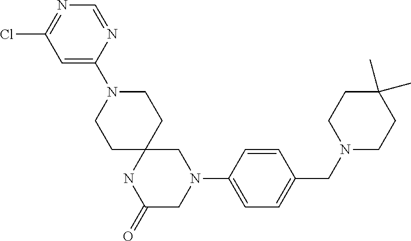 [SMILES: CC1(C)CCN(Cc2ccc(N3CC(=O)NC4(CCN(c5cc(Cl)ncn5)CC4)C3)cc2)CC1] | 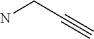 |
| | | 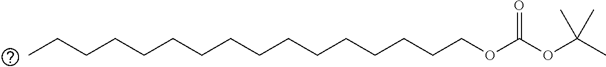 [SMILES: CCCCCCCCCCCCCCCCOC(=O)OC(C)(C)C] | 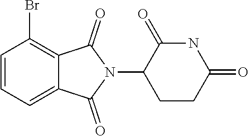 [SMILES: O=C1CCC(N2C(=O)c3cccc(Br)c3C2=O)C(=O)N1] |
| 362 | 7 | 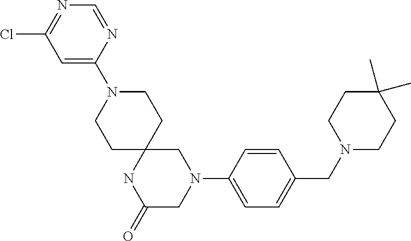 [SMILES: CC1(C)CCN(Cc2ccc(N3CC(=O)NC4(CCN(c5cc(Cl)ncn5)CC4)C3)cc2)CC1] | 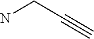 |
| | | 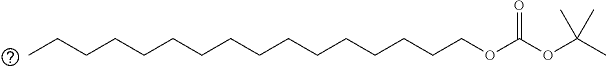 [SMILES: CCCCCCCCCCCCCCCCOC(=O)OC(C)(C)C] | 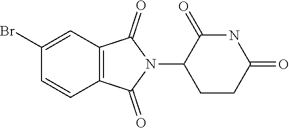 [SMILES: O=C1CCC(N2C(=O)c3ccc(Br)cc3C2=O)C(=O)N1] |
| 363 | 7 | 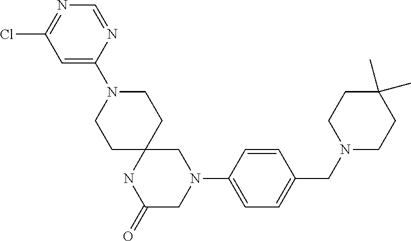 [SMILES: CC1(C)CCN(Cc2ccc(N3CC(=O)NC4(CCN(c5cc(Cl)ncn5)CC4)C3)cc2)CC1] | 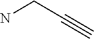 |
| | | 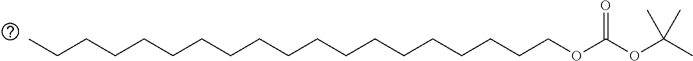 [SMILES: CCCCCCCCCCCCCCCCCCCOC(=O)OC(C)(C)C] | 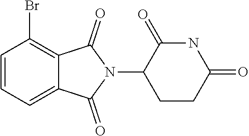 [SMILES: O=C1CCC(N2C(=O)c3cccc(Br)c3C2=O)C(=O)N1] |
| 364 | 7 | 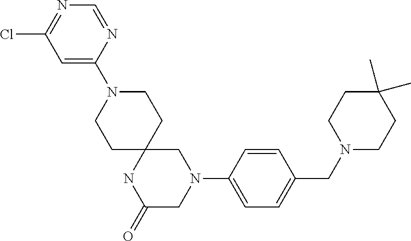 [SMILES: CC1(C)CCN(Cc2ccc(N3CC(=O)NC4(CCN(c5cc(Cl)ncn5)CC4)C3)cc2)CC1] | 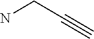 |
| | | 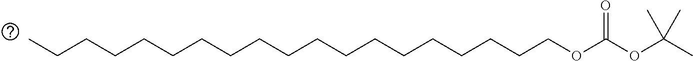 [SMILES: CCCCCCCCCCCCCCCCCCCOC(=O)OC(C)(C)C] | 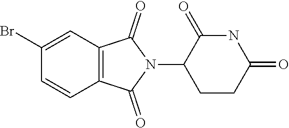 [SMILES: O=C1CCC(N2C(=O)c3ccc(Br)cc3C2=O)C(=O)N1] |

| Ex- | |
|---|---|
| am- | |
| ple | PROTAC |
| 1 | 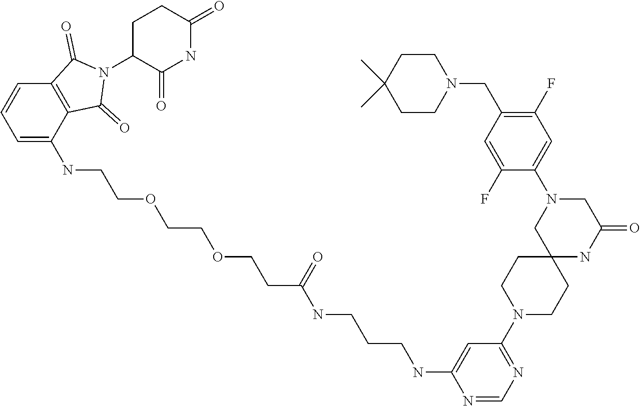 [SMILES: CC1(C)CCN(Cc2cc(F)c(N3CC(=O)NC4(CCN(c5cc(NCCCNC(=O)CCOCCOCCNc6cccc7c6C(=O)N(C6CCC(=O)NC6=O)C7=O)ncn5)CC4)C3)cc2F)CC1] |
| 2 | 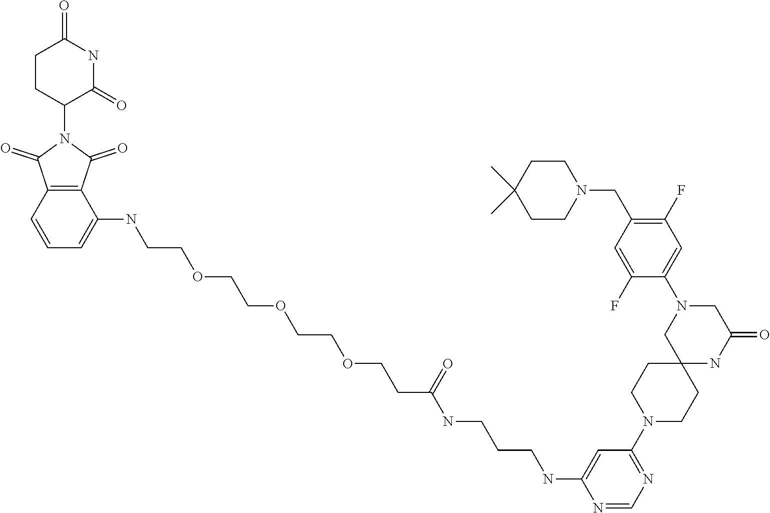 [SMILES: CC1(C)CCN(Cc2cc(F)c(N3CC(=O)NC4(CCN(c5cc(NCCCNC(=O)CCOCCOCCOCCNc6cccc7c6C(=O)N(C6CCC(=O)NC6=O)C7=O)ncn5)CC4)C3)cc2F)CC1] |
| 3 | 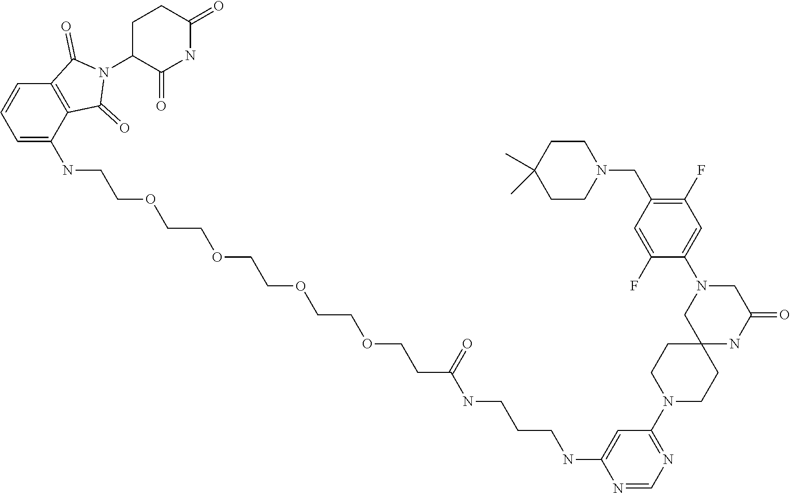 [SMILES: CC1(C)CCN(Cc2cc(F)c(N3CC(=O)NC4(CCN(c5cc(NCCCNC(=O)CCOCCOCCOCCOCCNc6cccc7c6C(=O)N(C6CCC(=O)NC6=O)C7=O)ncn5)CC4)C3)cc2F)CC1] |
| 4 | 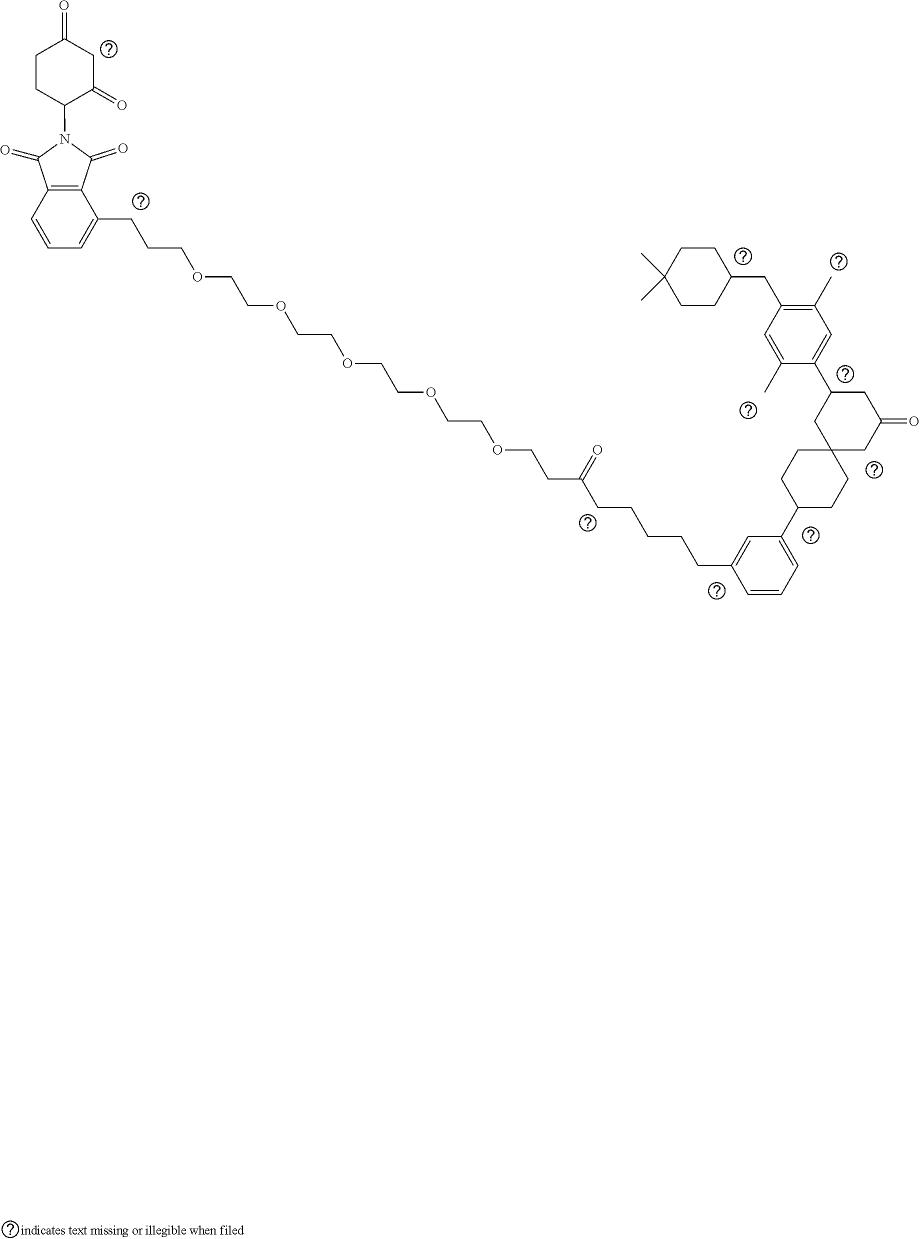 [SMILES: Cc1cc(C2CC(=O)CC3(CCC(c4cccc(CCCCCC(=O)CCOCCOCCOCCOCCOCCCc5cccc6c5C(=O)N(C5CCC(=O)CC5=O)C6=O)c4)CC3)C2)c(C)cc1CC1CCC(C)(C)CC1] |
| 5 | 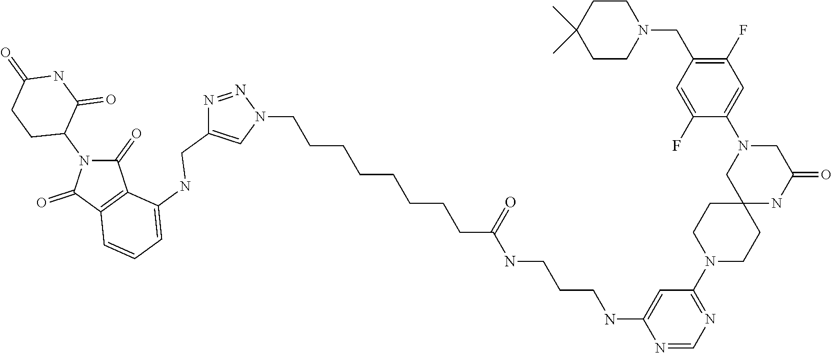 [SMILES: CC1(C)CCN(Cc2cc(F)c(N3CC(=O)NC4(CCN(c5cc(NCCCNC(=O)CCCCCCCCn6cc(CNc7cccc8c7C(=O)N(C7CCC(=O)NC7=O)C8=O)nn6)ncn5)CC4)C3)cc2F)CC1] |
| 6 | 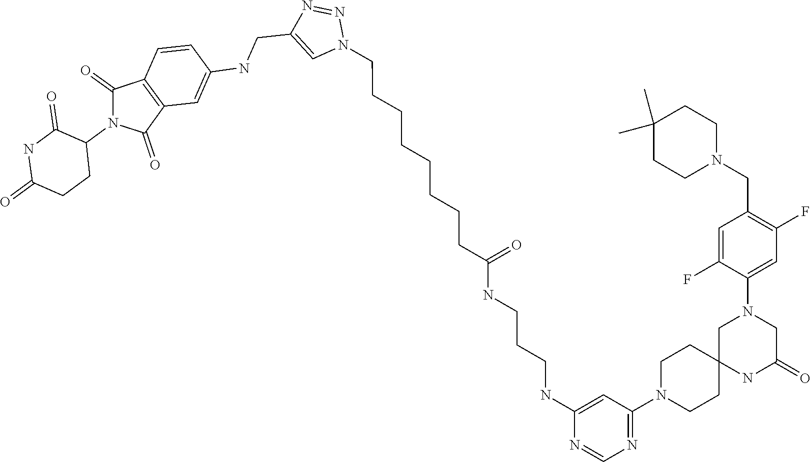 [SMILES: CC1(C)CCN(Cc2cc(F)c(N3CC(=O)NC4(CCN(c5cc(NCCCNC(=O)CCCCCCCCn6cc(CNc7ccc8c(c7)C(=O)N(C7CCC(=O)NC7=O)C8=O)nn6)ncn5)CC4)C3)cc2F)CC1] |
| 7 | 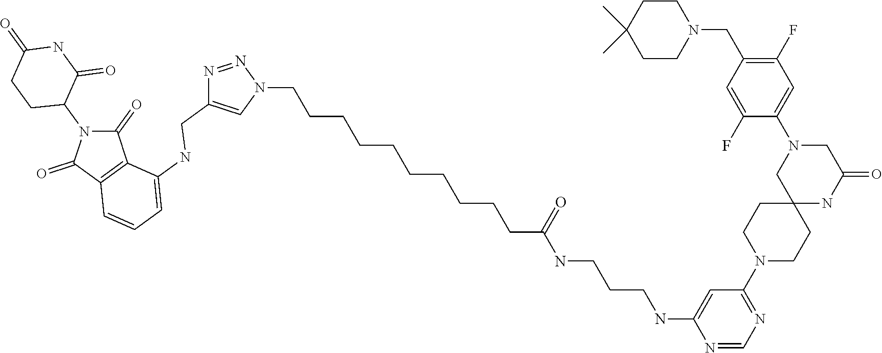 [SMILES: CC1(C)CCN(Cc2cc(F)c(N3CC(=O)NC4(CCN(c5cc(NCCCNC(=O)CCCCCCCCCCn6cc(CNc7cccc8c7C(=O)N(C7CCC(=O)NC7=O)C8=O)nn6)ncn5)CC4)C3)cc2F)CC1] |
| 8 | 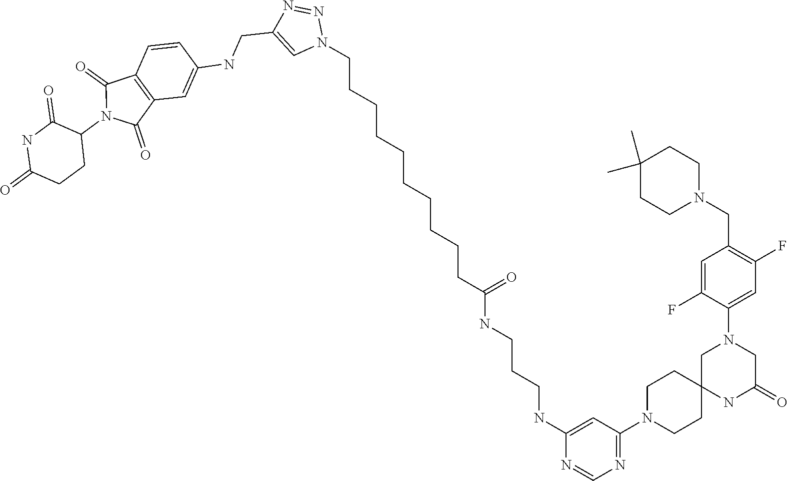 [SMILES: CC1(C)CCN(Cc2cc(F)c(N3CC(=O)NC4(CCN(c5cc(NCCCNC(=O)CCCCCCCCCCn6cc(CNc7ccc8c(c7)C(=O)N(C7CCC(=O)NC7=O)C8=O)nn6)ncn5)CC4)C3)cc2F)CC1] |
| 9 | 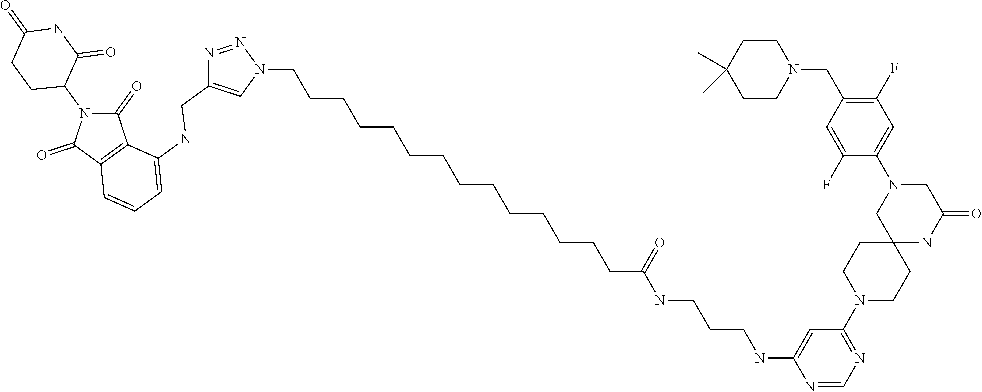 [SMILES: CC1(C)CCN(Cc2cc(F)c(N3CC(=O)NC4(CCN(c5cc(NCCCNC(=O)CCCCCCCCCCCCCCn6cc(CNc7cccc8c7C(=O)N(C7CCC(=O)NC7=O)C8=O)nn6)ncn5)CC4)C3)cc2F)CC1] |
| 10 | 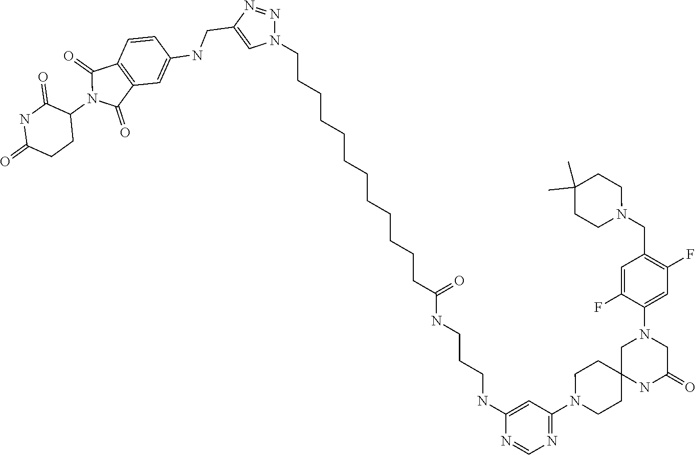 [SMILES: CC1(C)CCN(Cc2cc(F)c(N3CC(=O)NC4(CCN(c5cc(NCCCNC(=O)CCCCCCCCCCCCn6cc(CNc7ccc8c(c7)C(=O)N(C7CCC(=O)NC7=O)C8=O)nn6)ncn5)CC4)C3)cc2F)CC1] |
| 11 | 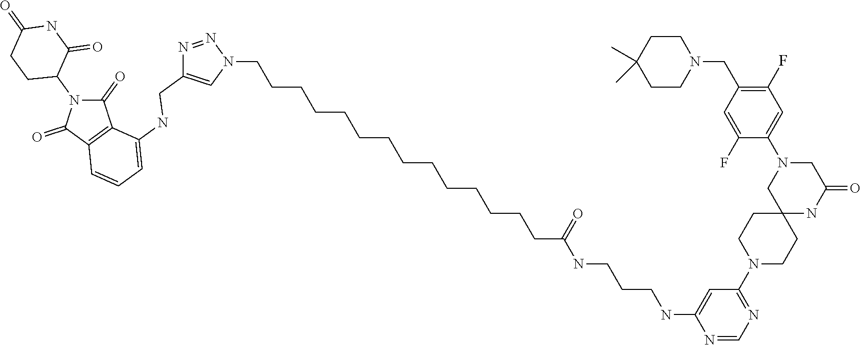 [SMILES: CC1(C)CCN(Cc2cc(F)c(N3CC(=O)NC4(CCN(c5cc(NCCCNC(=O)CCCCCCCCCCCCCCn6cc(CNc7cccc8c7C(=O)N(C7CCC(=O)NC7=O)C8=O)nn6)ncn5)CC4)C3)cc2F)CC1] |
| 12 | 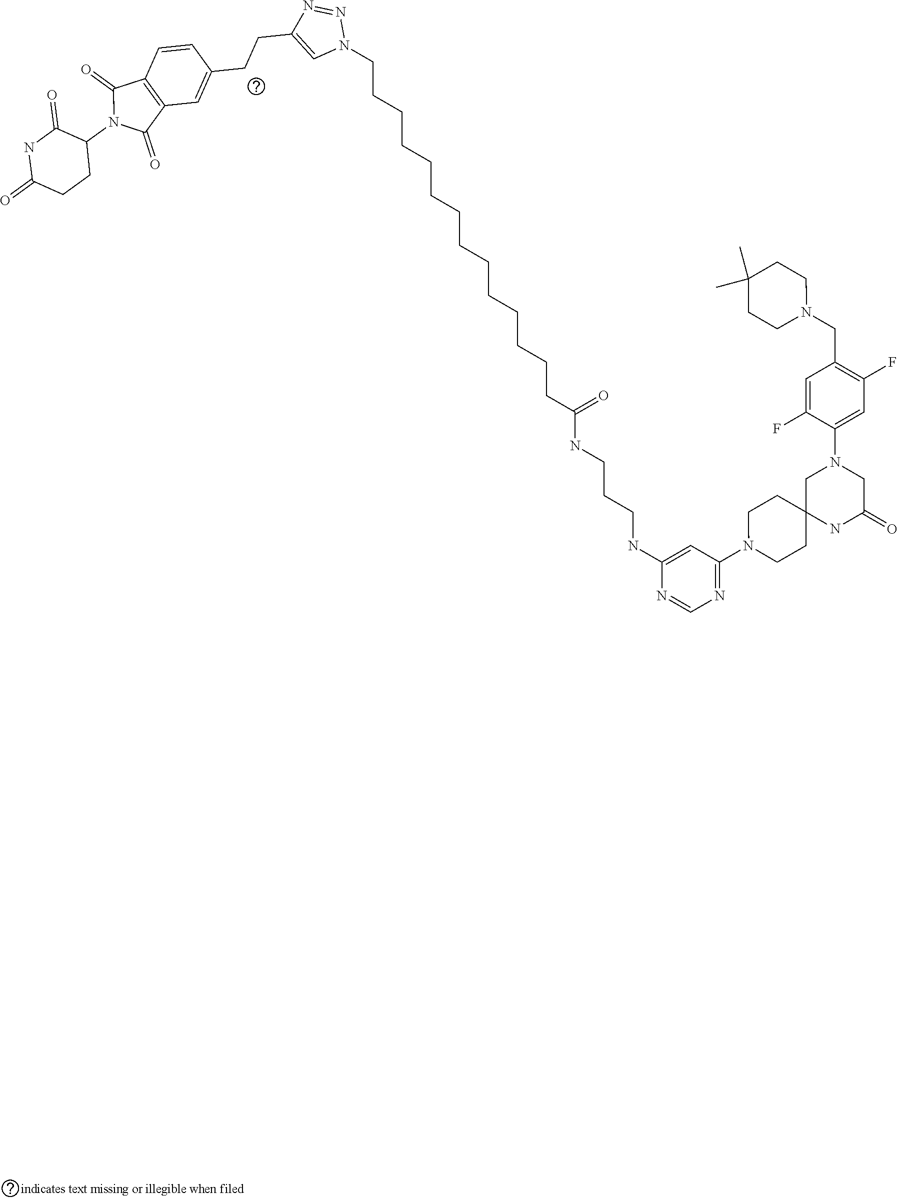 [SMILES: CC1(C)CCN(Cc2cc(F)c(N3CC(=O)NC4(CCN(c5cc(NCCCNC(=O)CCCCCCCCCCCCCCn6cc(CCc7ccc8c(c7)C(=O)N(C7CCC(=O)NC7=O)C8=O)nn6)ncn5)CC4)C3)cc2F)CC1] |
| 13 | 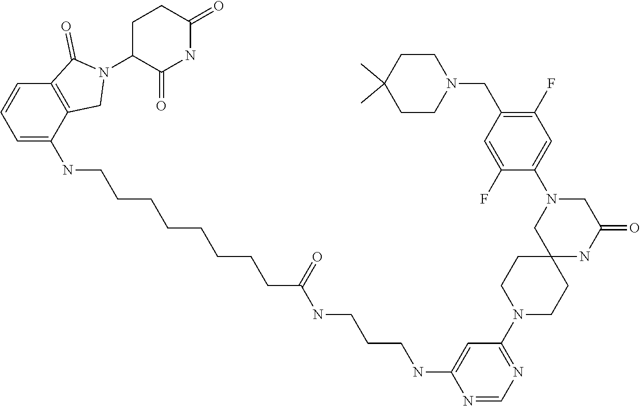 [SMILES: CC1(C)CCN(Cc2cc(F)c(N3CC(=O)NC4(CCN(c5cc(NCCCNC(=O)CCCCCCCCNc6cccc7c6CN(C6CCC(=O)NC6=O)C7=O)ncn5)CC4)C3)cc2F)CC1] |
| 14 | 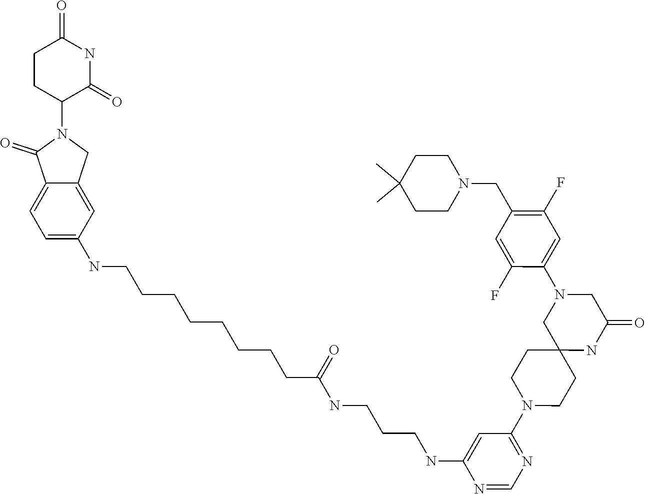 [SMILES: CC1(C)CCN(Cc2cc(F)c(N3CC(=O)NC4(CCN(c5cc(NCCCNC(=O)CCCCCCCCNc6ccc7c(c6)CN(C6CCC(=O)NC6=O)C7=O)ncn5)CC4)C3)cc2F)CC1] |
| 15 | 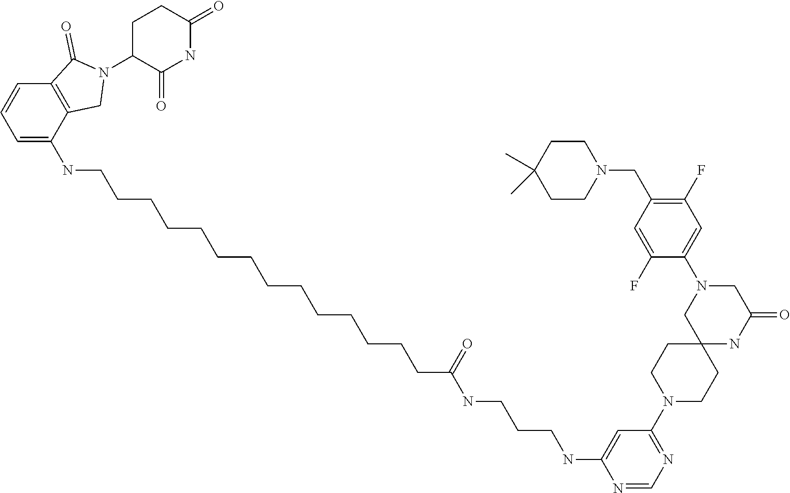 [SMILES: CC1(C)CCN(Cc2cc(F)c(N3CC(=O)NC4(CCN(c5cc(NCCCNC(=O)CCCCCCCCCCCCCCNc6cccc7c6CN(C6CCC(=O)NC6=O)C7=O)ncn5)CC4)C3)cc2F)CC1] |
| 16 | 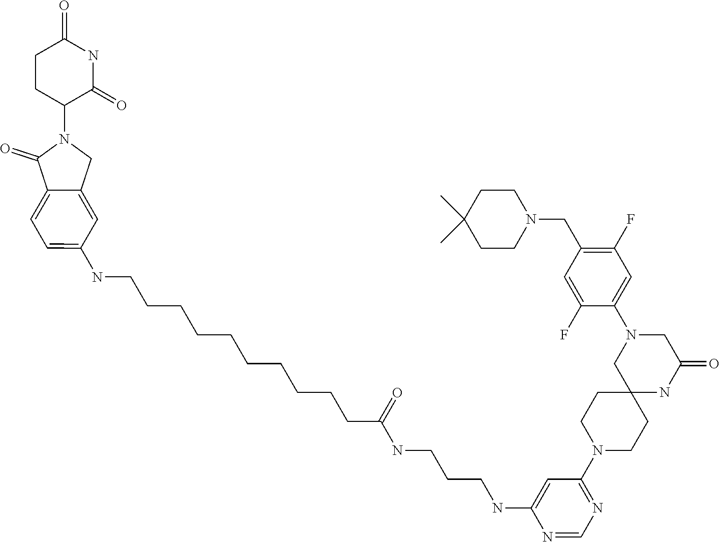 [SMILES: CC1(C)CCN(Cc2cc(F)c(N3CC(=O)NC4(CCN(c5cc(NCCCNC(=O)CCCCCCCCCCNc6ccc7c(c6)CN(C6CCC(=O)NC6=O)C7=O)ncn5)CC4)C3)cc2F)CC1] |
| 17 | 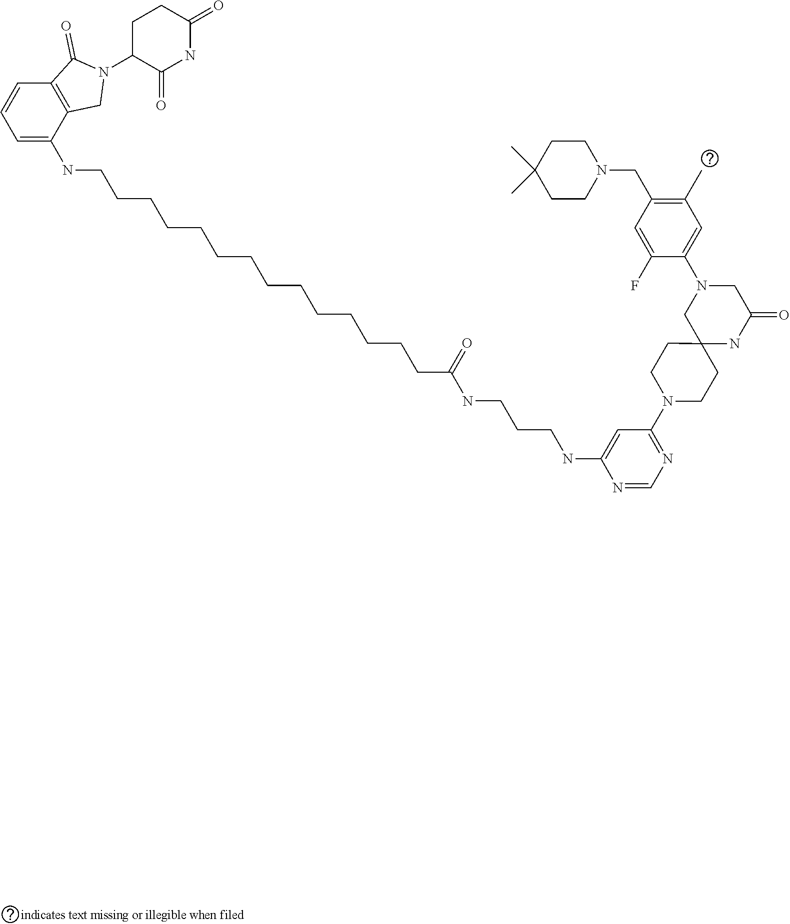 [SMILES: Cc1cc(N2CC(=O)NC3(CCN(c4cc(NCCCNC(=O)CCCCCCCCCCCCCCNc5cccc6c5CN(C5CCC(=O)NC5=O)C6=O)ncn4)CC3)C2)c(F)cc1CN1CCC(C)(C)CC1] |
| 18 | 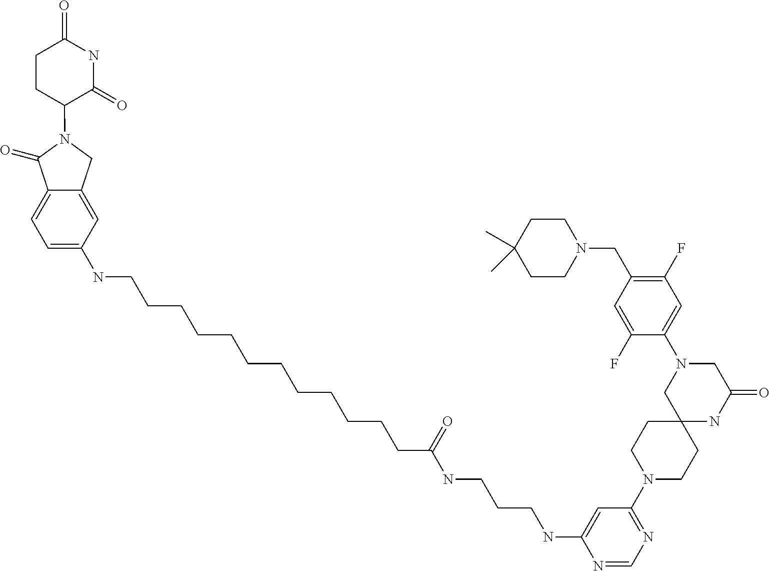 [SMILES: CC1(C)CCN(Cc2cc(F)c(N3CC(=O)NC4(CCN(c5cc(NCCCNC(=O)CCCCCCCCCCCCNc6ccc7c(c6)CN(C6CCC(=O)NC6=O)C7=O)ncn5)CC4)C3)cc2F)CC1] |
| 19 | 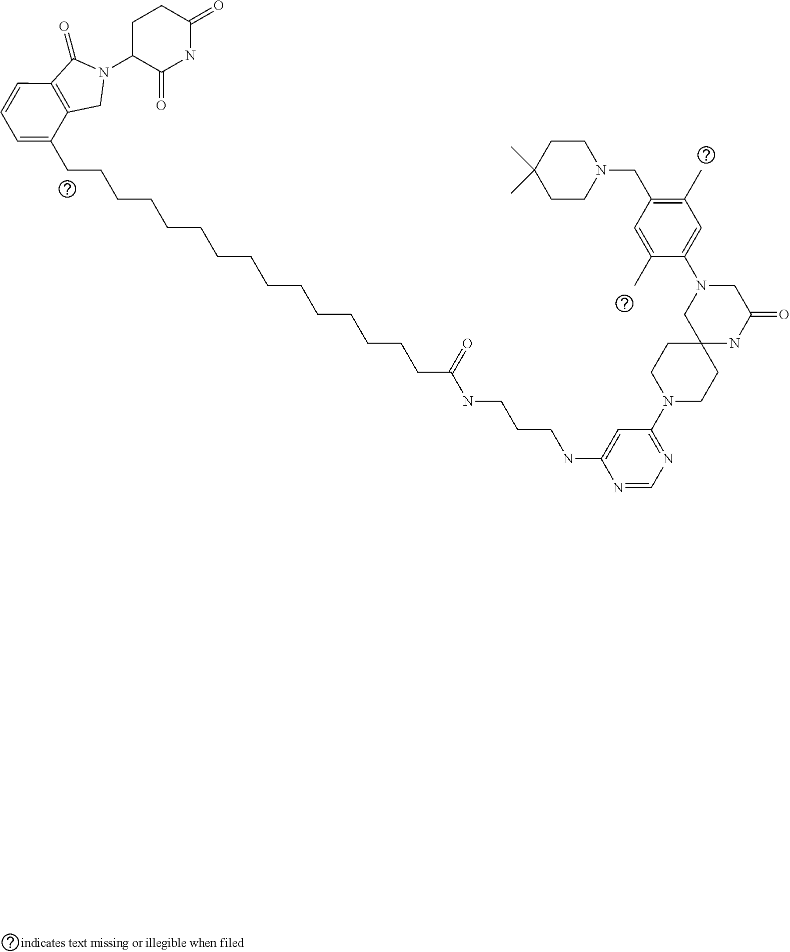 [SMILES: Cc1cc(N2CC(=O)NC3(CCN(c4cc(NCCCNC(=O)CCCCCCCCCCCCCCCc5cccc6c5CN(C5CCC(=O)NC5=O)C6=O)ncn4)CC3)C2)c(C)cc1CN1CCC(C)(C)CC1] |
| 20 | 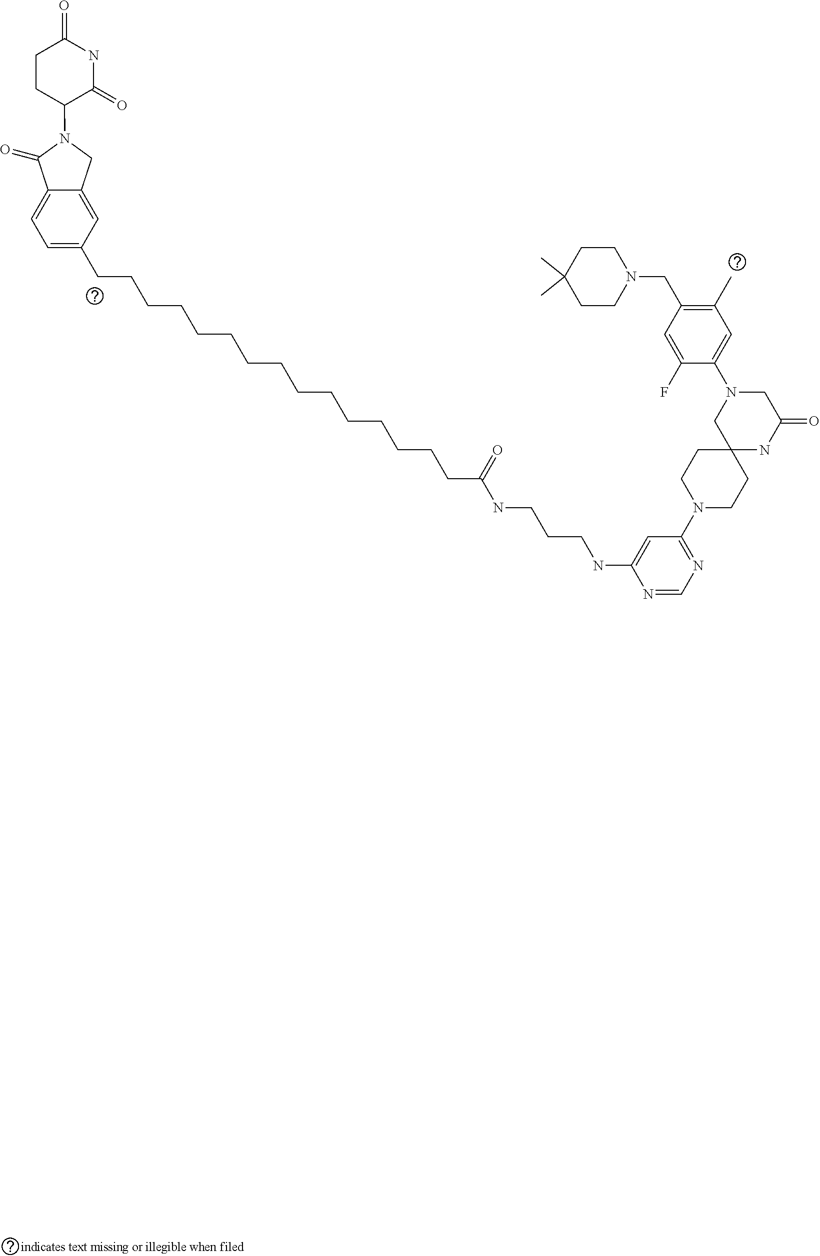 [SMILES: Cc1cc(N2CC(=O)NC3(CCN(c4cc(NCCCNC(=O)CCCCCCCCCCCCCCCc5ccc6c(c5)CN(C5CCC(=O)NC5=O)C6=O)ncn4)CC3)C2)c(F)cc1CN1CCC(C)(C)CC1] |
| 21 | 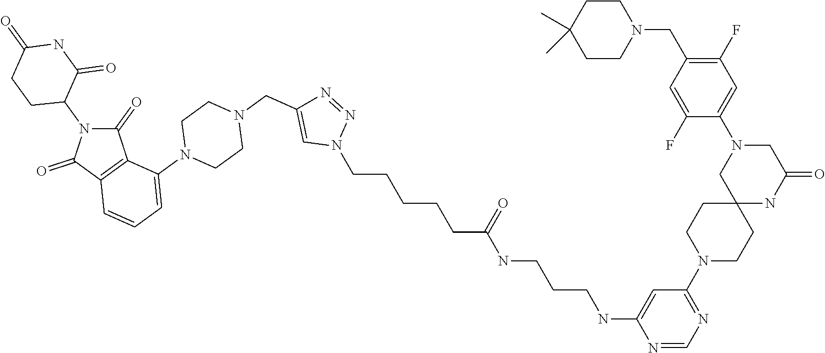 [SMILES: CC1(C)CCN(Cc2cc(F)c(N3CC(=O)NC4(CCN(c5cc(NCCCNC(=O)CCCCCn6cc(CN7CCN(c8cccc9c8C(=O)N(C8CCC(=O)NC8=O)C9=O)CC7)nn6)ncn5)CC4)C3)cc2F)CC1] |
| 22 | 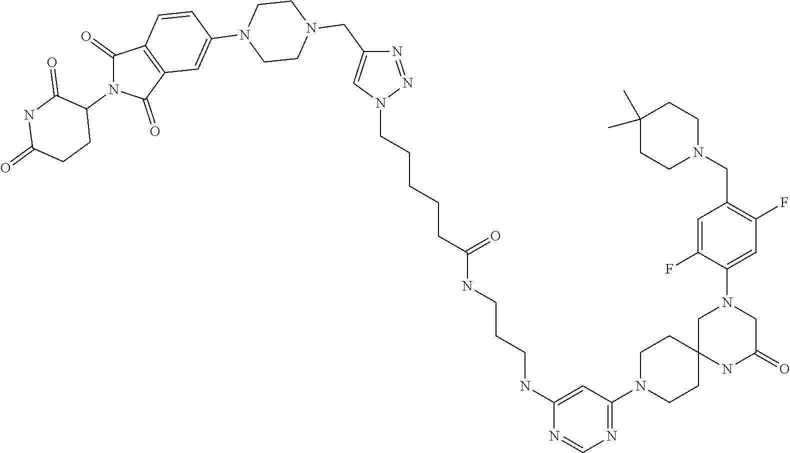 [SMILES: CC1(C)CCN(Cc2cc(F)c(N3CC(=O)NC4(CCN(c5cc(NCCCNC(=O)CCCCCn6cc(CN7CCN(c8ccc9c(c8)C(=O)N(C8CCC(=O)NC8=O)C9=O)CC7)nn6)ncn5)CC4)C3)cc2F)CC1] |
| 23 | 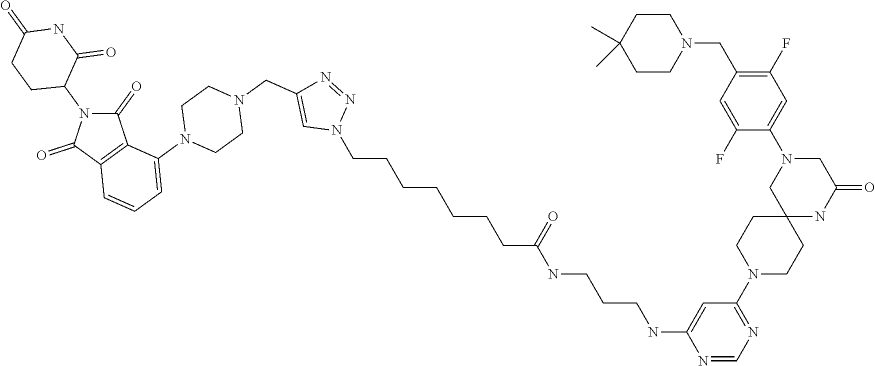 [SMILES: CC1(C)CCN(Cc2cc(F)c(N3CC(=O)NC4(CCN(c5cc(NCCCNC(=O)CCCCCCCn6cc(CN7CCN(c8cccc9c8C(=O)N(C8CCC(=O)NC8=O)C9=O)CC7)nn6)ncn5)CC4)C3)cc2F)CC1] |
| 24 | 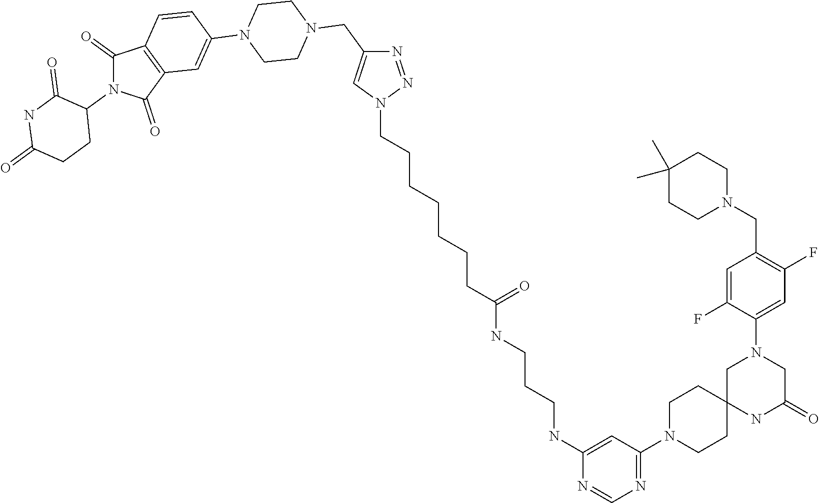 [SMILES: CC1(C)CCN(Cc2cc(F)c(N3CC(=O)NC4(CCN(c5cc(NCCCNC(=O)CCCCCCCn6cc(CN7CCN(c8ccc9c(c8)C(=O)N(C8CCC(=O)NC8=O)C9=O)CC7)nn6)ncn5)CC4)C3)cc2F)CC1] |
| 25 | 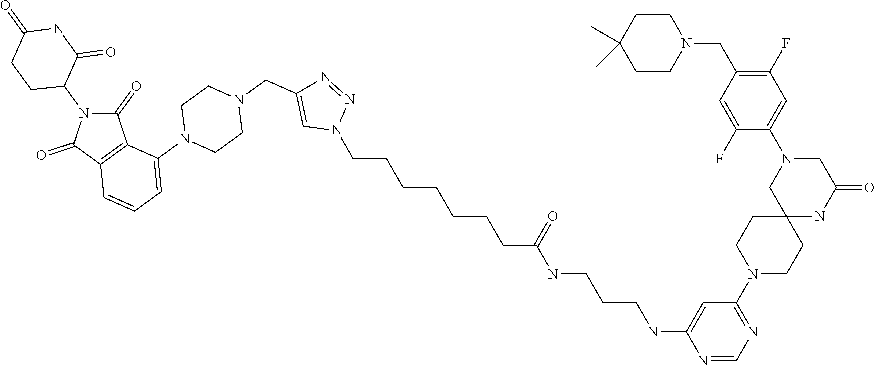 [SMILES: CC1(C)CCN(Cc2cc(F)c(N3CC(=O)NC4(CCN(c5cc(NCCCNC(=O)CCCCCCCn6cc(CN7CCN(c8cccc9c8C(=O)N(C8CCC(=O)NC8=O)C9=O)CC7)nn6)ncn5)CC4)C3)cc2F)CC1] |
| 26 | 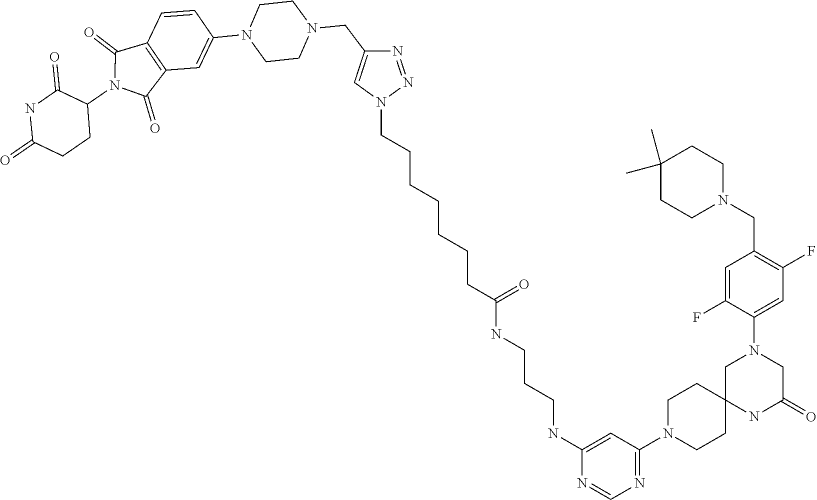 [SMILES: CC1(C)CCN(Cc2cc(F)c(N3CC(=O)NC4(CCN(c5cc(NCCCNC(=O)CCCCCCCn6cc(CN7CCN(c8ccc9c(c8)C(=O)N(C8CCC(=O)NC8=O)C9=O)CC7)nn6)ncn5)CC4)C3)cc2F)CC1] |
| 27 | 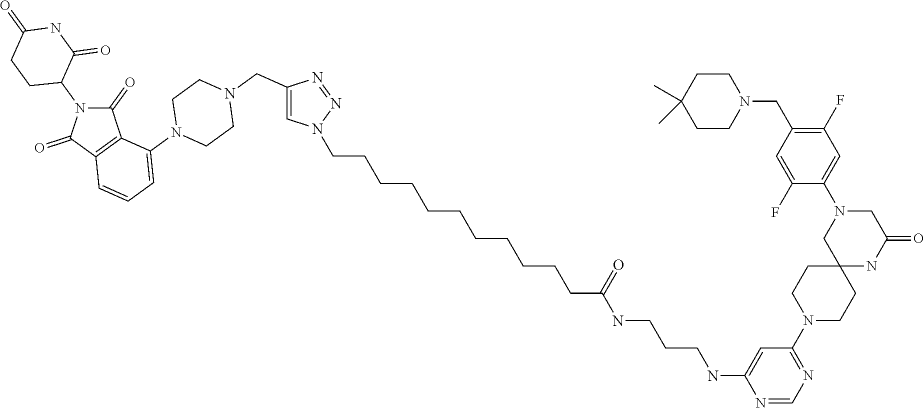 [SMILES: CC1(C)CCN(Cc2cc(F)c(N3CC(=O)NC4(CCN(c5cc(NCCCNC(=O)CCCCCCCCCCCn6cc(CN7CCN(c8cccc9c8C(=O)N(C8CCC(=O)NC8=O)C9=O)CC7)nn6)ncn5)CC4)C3)cc2F)CC1] |
| 28 | 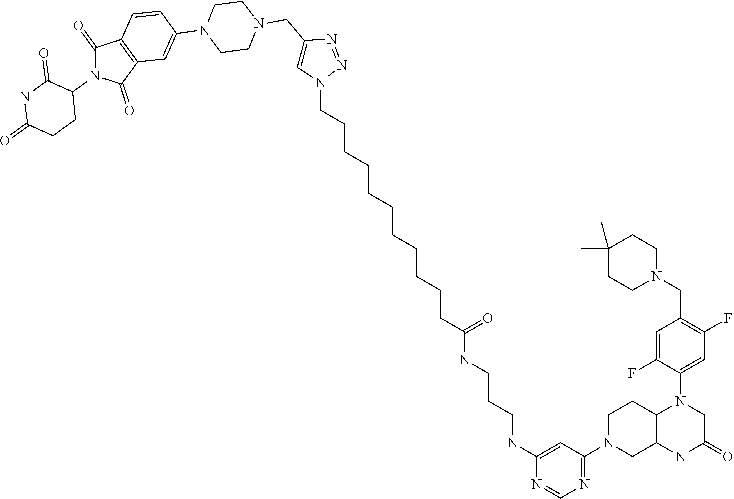 [SMILES: CC1(C)CCN(Cc2cc(F)c(N3CC(=O)NC4CN(c5cc(NCCCNC(=O)CCCCCCCCCCCn6cc(CN7CCN(c8ccc9c(c8)C(=O)N(C8CCC(=O)NC8=O)C9=O)CC7)nn6)ncn5)CCC43)cc2F)CC1] |
| 29 | 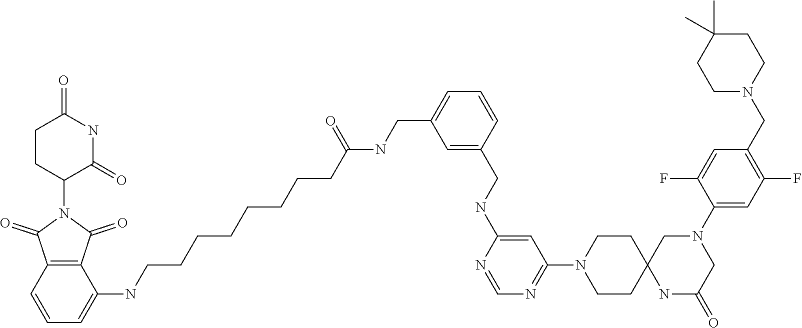 [SMILES: CC1(C)CCN(Cc2cc(F)c(N3CC(=O)NC4(CCN(c5cc(NCc6cccc(CNC(=O)CCCCCCCCNc7cccc8c7C(=O)N(C7CCC(=O)NC7=O)C8=O)c6)ncn5)CC4)C3)cc2F)CC1] |
| 30 | 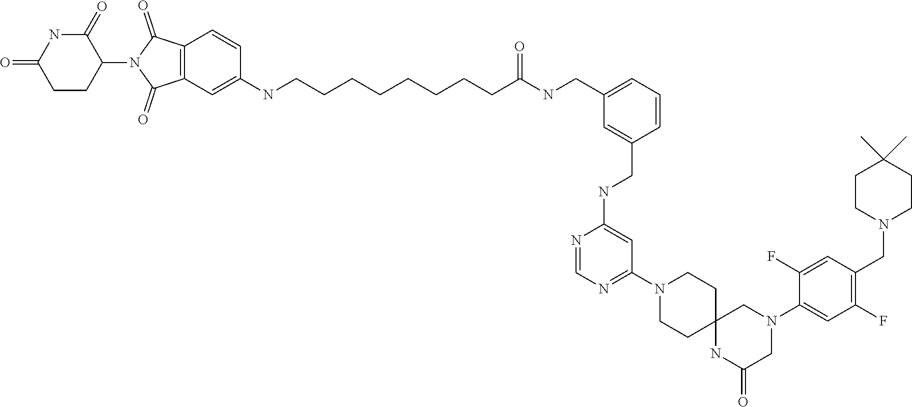 [SMILES: CC1(C)CCN(Cc2cc(F)c(N3CC(=O)NC4(CCN(c5cc(NCc6cccc(CNC(=O)CCCCCCCCNc7ccc8c(c7)C(=O)N(C7CCC(=O)NC7=O)C8=O)c6)ncn5)CC4)C3)cc2F)CC1] |
| 31 | 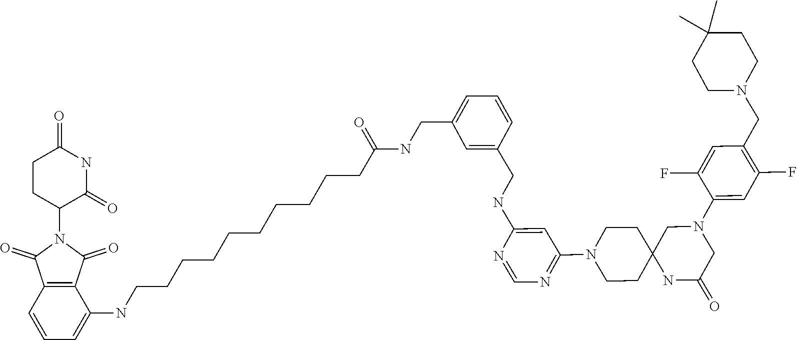 [SMILES: CC1(C)CCN(Cc2cc(F)c(N3CC(=O)NC4(CCN(c5cc(NCc6cccc(CNC(=O)CCCCCCCCCCNc7cccc8c7C(=O)N(C7CCC(=O)NC7=O)C8=O)c6)ncn5)CC4)C3)cc2F)CC1] |
| 32 | 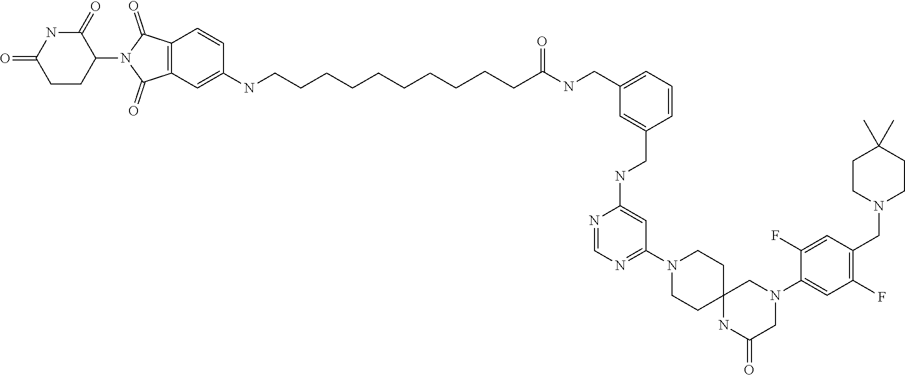 [SMILES: CC1(C)CCN(Cc2cc(F)c(N3CC(=O)NC4(CCN(c5cc(NCc6cccc(CNC(=O)CCCCCCCCCCNc7ccc8c(c7)C(=O)N(C7CCC(=O)NC7=O)C8=O)c6)ncn5)CC4)C3)cc2F)CC1] |
| 33 | 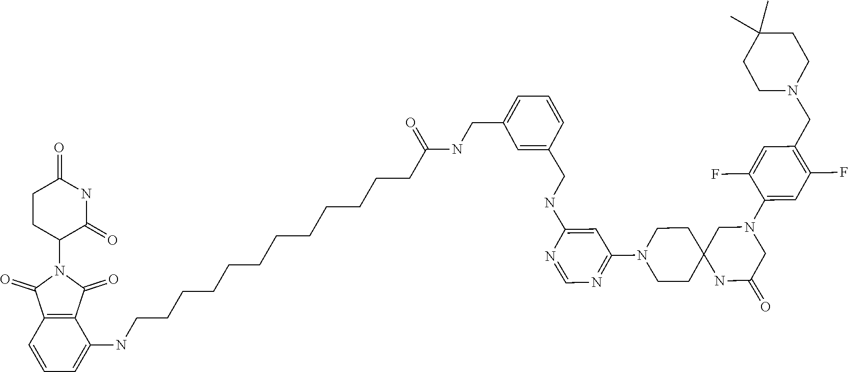 [SMILES: CC1(C)CCN(Cc2cc(F)c(N3CC(=O)NC4(CCN(c5cc(NCc6cccc(CNC(=O)CCCCCCCCCCCCNc7cccc8c7C(=O)N(C7CCC(=O)NC7=O)C8=O)c6)ncn5)CC4)C3)cc2F)CC1] |
| 34 | 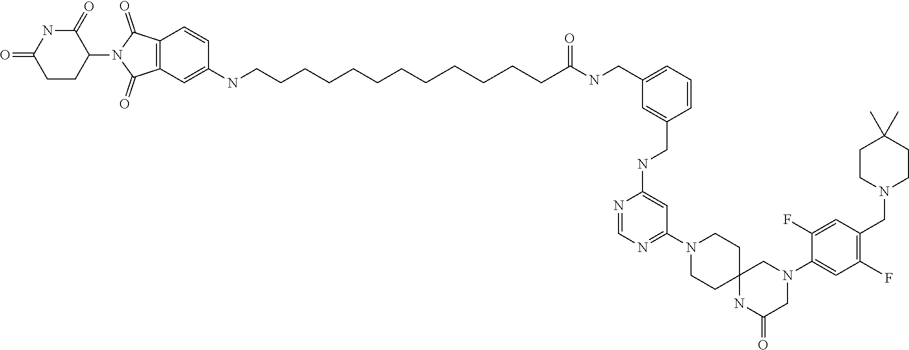 [SMILES: CC1(C)CCN(Cc2cc(F)c(N3CC(=O)NC4(CCN(c5cc(NCc6cccc(CNC(=O)CCCCCCCCCCCCNc7ccc8c(c7)C(=O)N(C7CCC(=O)NC7=O)C8=O)c6)ncn5)CC4)C3)cc2F)CC1] |
| 35 | 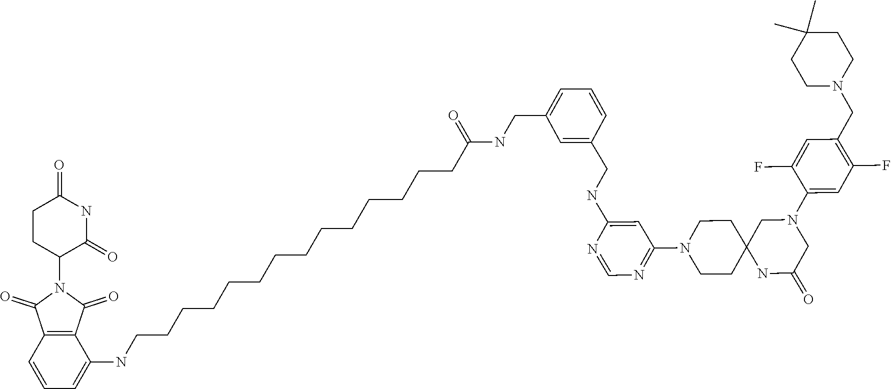 [SMILES: CC1(C)CCN(Cc2cc(F)c(N3CC(=O)NC4(CCN(c5cc(NCc6cccc(CNC(=O)CCCCCCCCCCCCCCNc7cccc8c7C(=O)N(C7CCC(=O)NC7=O)C8=O)c6)ncn5)CC4)C3)cc2F)CC1] |
| 36 | 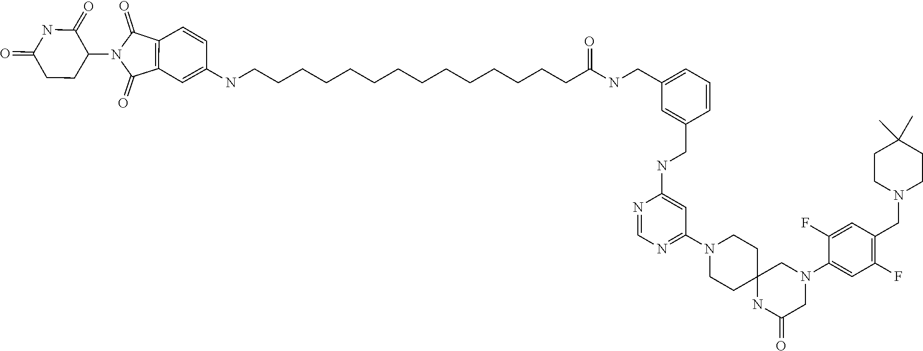 [SMILES: CC1(C)CCN(Cc2cc(F)c(N3CC(=O)NC4(CCN(c5cc(NCc6cccc(CNC(=O)CCCCCCCCCCCCCCNc7ccc8c(c7)C(=O)N(C7CCC(=O)NC7=O)C8=O)c6)ncn5)CC4)C3)cc2F)CC1] |
| 37 | 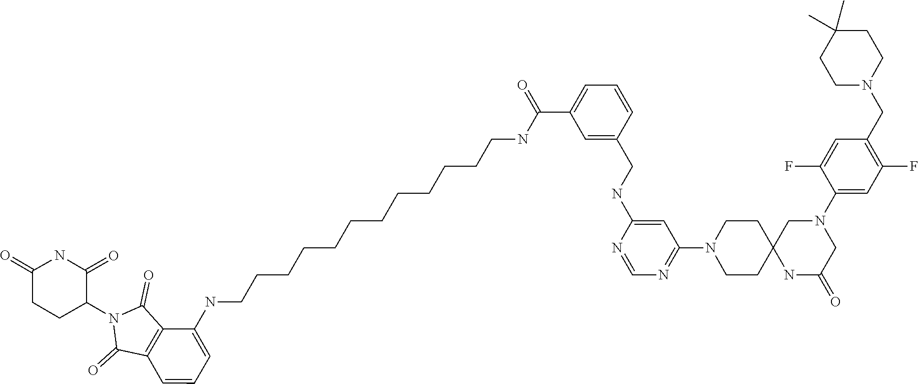 [SMILES: CC1(C)CCN(Cc2cc(F)c(N3CC(=O)NC4(CCN(c5cc(NCc6cccc(C(=O)NCCCCCCCCCCCCNc7cccc8c7C(=O)N(C7CCC(=O)NC7=O)C8=O)c6)ncn5)CC4)C3)cc2F)CC1] |
| 38 | 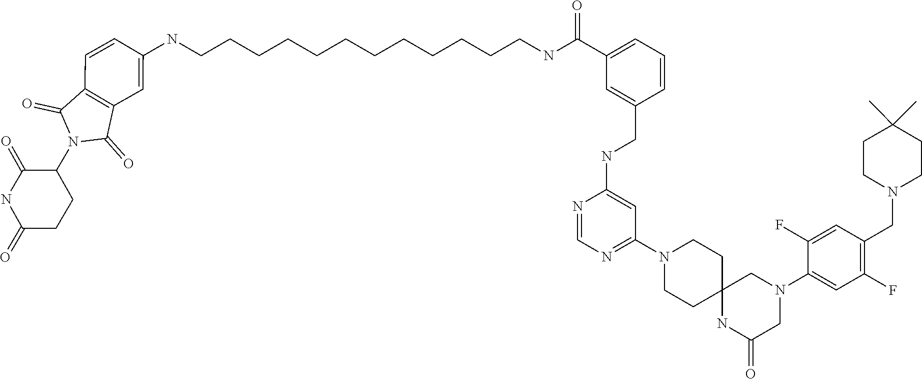 [SMILES: CC1(C)CCN(Cc2cc(F)c(N3CC(=O)NC4(CCN(c5cc(NCc6cccc(C(=O)NCCCCCCCCCCCCNc7ccc8c(c7)C(=O)N(C7CCC(=O)NC7=O)C8=O)c6)ncn5)CC4)C3)cc2F)CC1] |
| 39 | 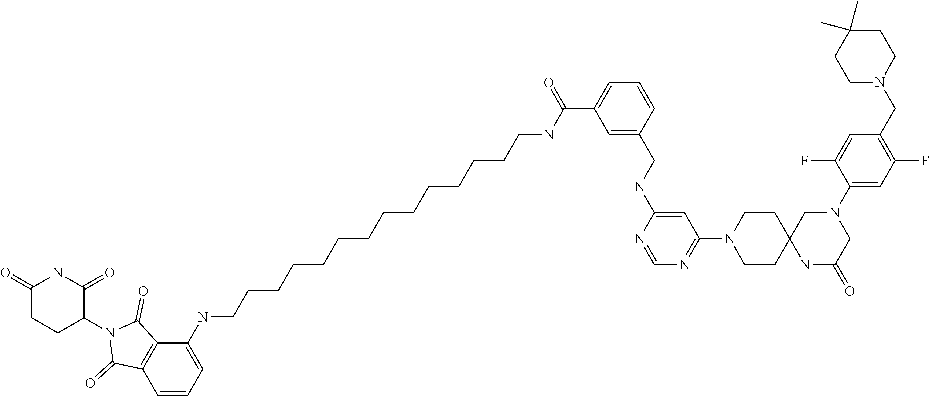 [SMILES: CC1(C)CCN(Cc2cc(F)c(N3CC(=O)NC4(CCN(c5cc(NCc6cccc(C(=O)NCCCCCCCCCCCCCCNc7cccc8c7C(=O)N(C7CCC(=O)NC7=O)C8=O)c6)ncn5)CC4)C3)cc2F)CC1] |
| 40 | 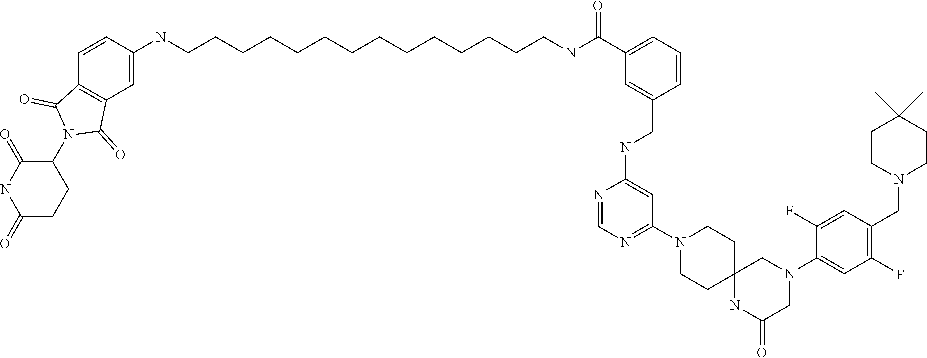 [SMILES: CC1(C)CCN(Cc2cc(F)c(N3CC(=O)NC4(CCN(c5cc(NCc6cccc(C(=O)NCCCCCCCCCCCCCCNc7ccc8c(c7)C(=O)N(C7CCC(=O)NC7=O)C8=O)c6)ncn5)CC4)C3)cc2F)CC1] |
| 41 | 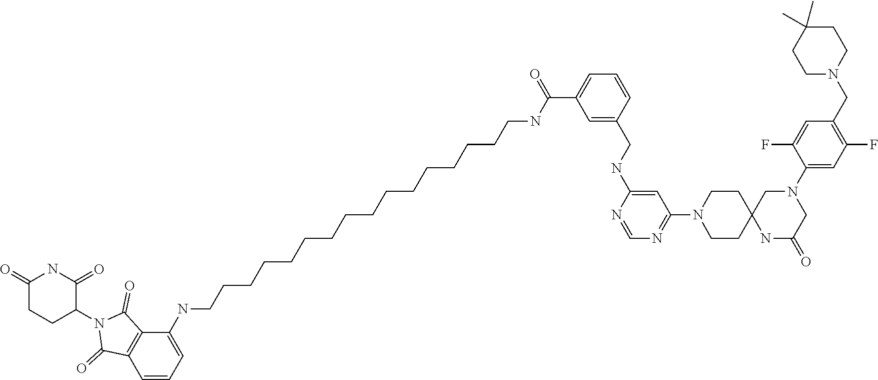 [SMILES: CC1(C)CCN(Cc2cc(F)c(N3CC(=O)NC4(CCN(c5cc(NCc6cccc(C(=O)NCCCCCCCCCCCCCCCCNc7cccc8c7C(=O)N(C7CCC(=O)NC7=O)C8=O)c6)ncn5)CC4)C3)cc2F)CC1] |
| 42 | 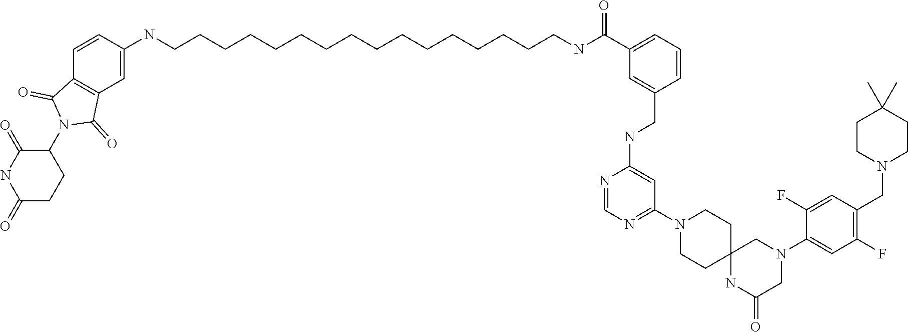 [SMILES: CC1(C)CCN(Cc2cc(F)c(N3CC(=O)NC4(CCN(c5cc(NCc6cccc(C(=O)NCCCCCCCCCCCCCCCCNc7ccc8c(c7)C(=O)N(C7CCC(=O)NC7=O)C8=O)c6)ncn5)CC4)C3)cc2F)CC1] |
| 43 | 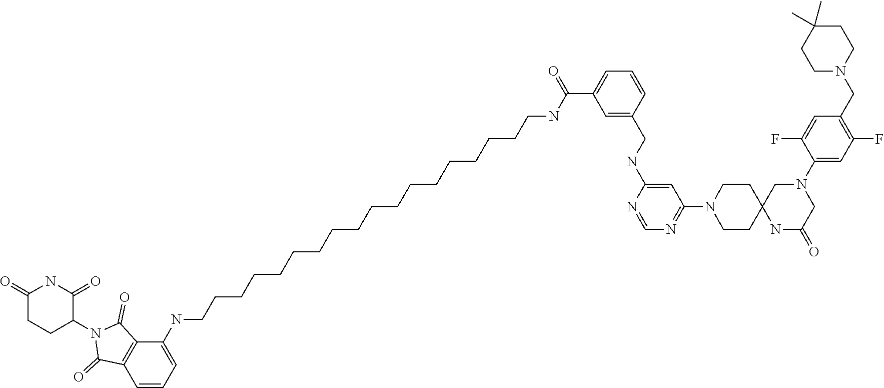 [SMILES: CC1(C)CCN(Cc2cc(F)c(N3CC(=O)NC4(CCN(c5cc(NCc6cccc(C(=O)NCCCCCCCCCCCCCCCCCCNc7cccc8c7C(=O)N(C7CCC(=O)NC7=O)C8=O)c6)ncn5)CC4)C3)cc2F)CC1] |
| 44 | 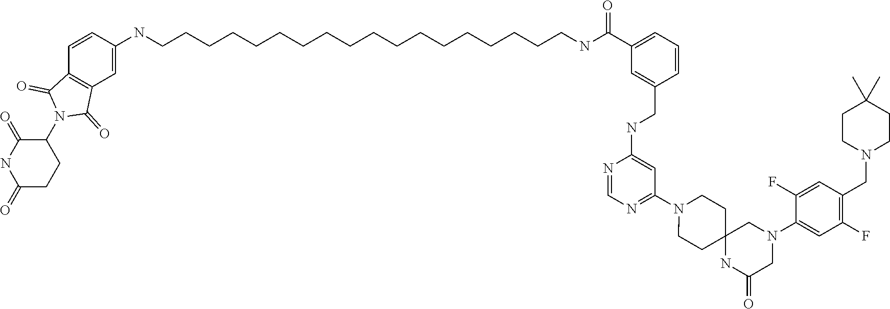 [SMILES: CC1(C)CCN(Cc2cc(F)c(N3CC(=O)NC4(CCN(c5cc(NCc6cccc(C(=O)NCCCCCCCCCCCCCCCCCCNc7ccc8c(c7)C(=O)N(C7CCC(=O)NC7=O)C8=O)c6)ncn5)CC4)C3)cc2F)CC1] |
| 45 | 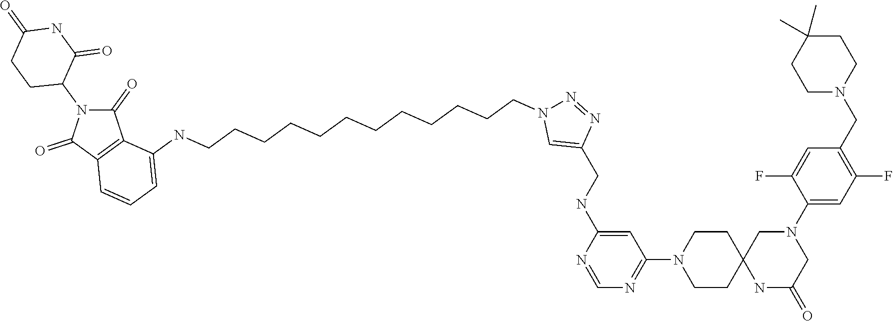 [SMILES: CC1(C)CCN(Cc2cc(F)c(N3CC(=O)NC4(CCN(c5cc(NCc6cn(CCCCCCCCCCCCNc7cccc8c7C(=O)N(C7CCC(=O)NC7=O)C8=O)nn6)ncn5)CC4)C3)cc2F)CC1] |
| 46 | 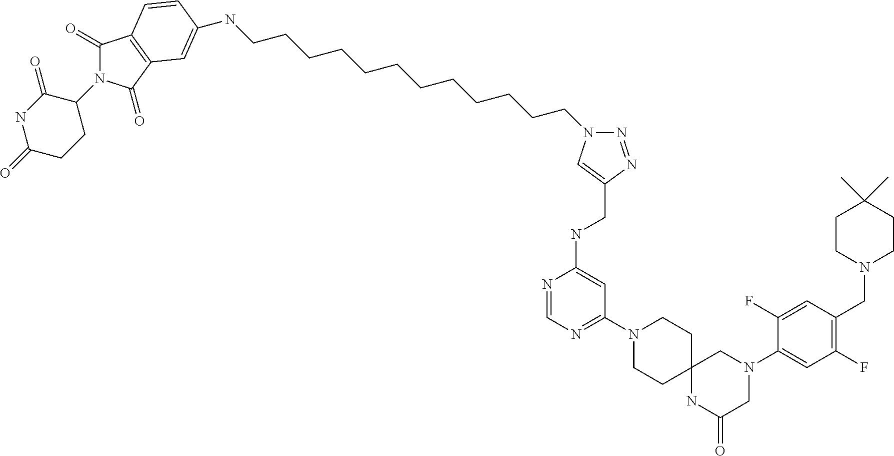 [SMILES: CC1(C)CCN(Cc2cc(F)c(N3CC(=O)NC4(CCN(c5cc(NCc6cn(CCCCCCCCCCCCNc7ccc8c(c7)C(=O)N(C7CCC(=O)NC7=O)C8=O)nn6)ncn5)CC4)C3)cc2F)CC1] |
| 47 | 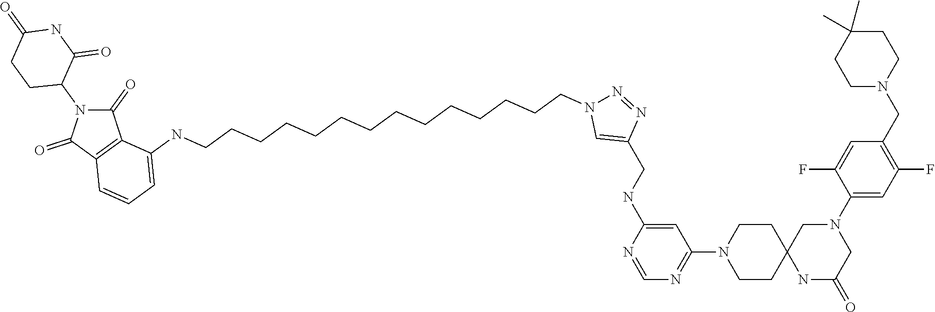 [SMILES: CC1(C)CCN(Cc2cc(F)c(N3CC(=O)NC4(CCN(c5cc(NCc6cn(CCCCCCCCCCCCCCNc7cccc8c7C(=O)N(C7CCC(=O)NC7=O)C8=O)nn6)ncn5)CC4)C3)cc2F)CC1] |
| 48 | 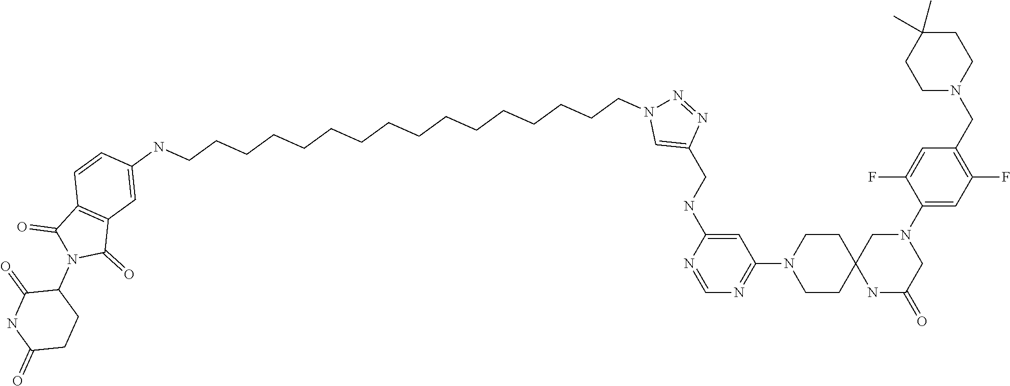 [SMILES: CC1(C)CCN(Cc2cc(F)c(N3CC(=O)NC4(CCN(c5cc(NCc6cn(CCCCCCCCCCCCCCCCNc7ccc8c(c7)C(=O)N(C7CCC(=O)NC7=O)C8=O)nn6)ncn5)CC4)C3)cc2F)CC1] |
| 49 | 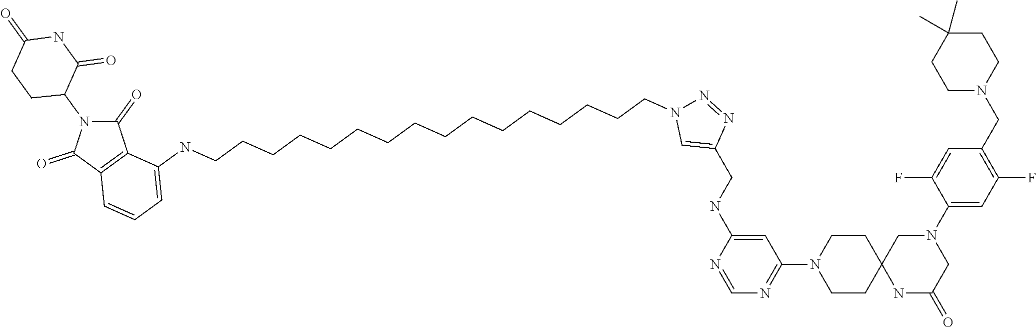 [SMILES: CC1(C)CCN(Cc2cc(F)c(N3CC(=O)NC4(CCN(c5cc(NCc6cn(CCCCCCCCCCCCCCCCNc7cccc8c7C(=O)N(C7CCC(=O)NC7=O)C8=O)nn6)ncn5)CC4)C3)cc2F)CC1] |
| 50 | 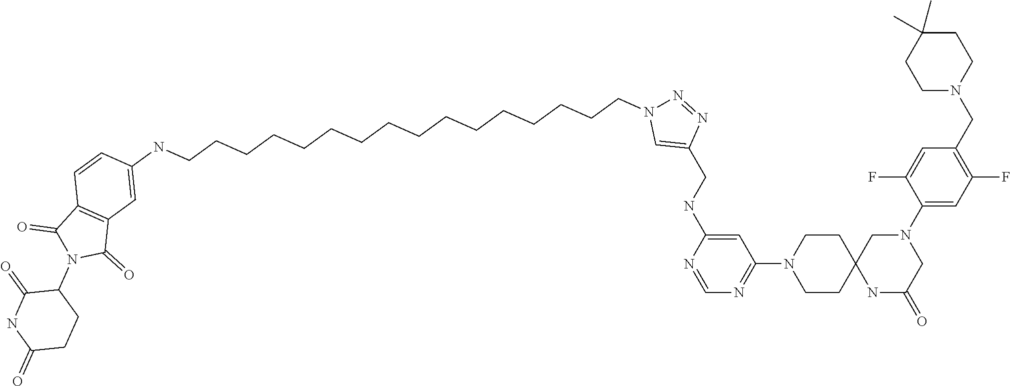 [SMILES: CC1(C)CCN(Cc2cc(F)c(N3CC(=O)NC4(CCN(c5cc(NCc6cn(CCCCCCCCCCCCCCCCNc7ccc8c(c7)C(=O)N(C7CCC(=O)NC7=O)C8=O)nn6)ncn5)CC4)C3)cc2F)CC1] |
| 51 | 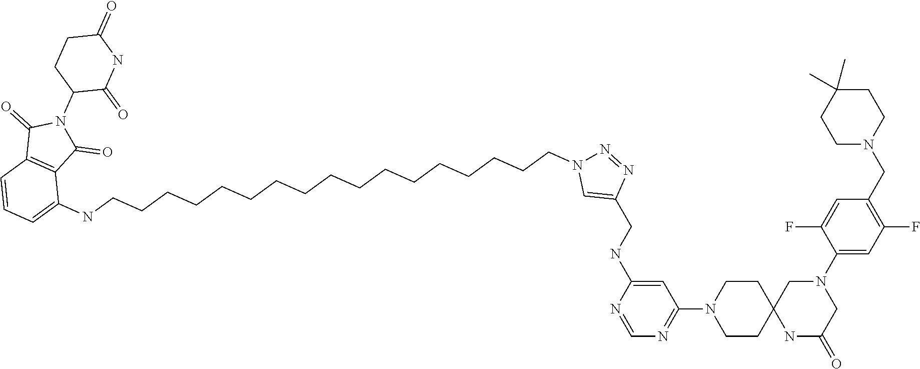 [SMILES: CC1(C)CCN(Cc2cc(F)c(N3CC(=O)NC4(CCN(c5cc(NCc6cn(CCCCCCCCCCCCCCCCCNc7cccc8c7C(=O)N(C7CCC(=O)NC7=O)C8=O)nn6)ncn5)CC4)C3)cc2F)CC1] |
| 52 | 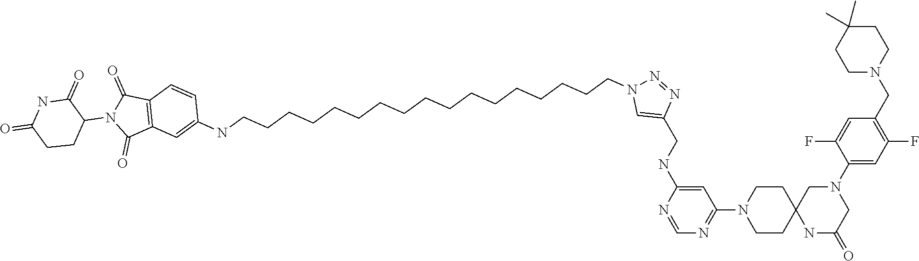 [SMILES: CC1(C)CCN(Cc2cc(F)c(N3CC(=O)NC4(CCN(c5cc(NCc6cn(CCCCCCCCCCCCCCCCCNc7ccc8c(c7)C(=O)N(C7CCC(=O)NC7=O)C8=O)nn6)ncn5)CC4)C3)cc2F)CC1] |
| 53 | 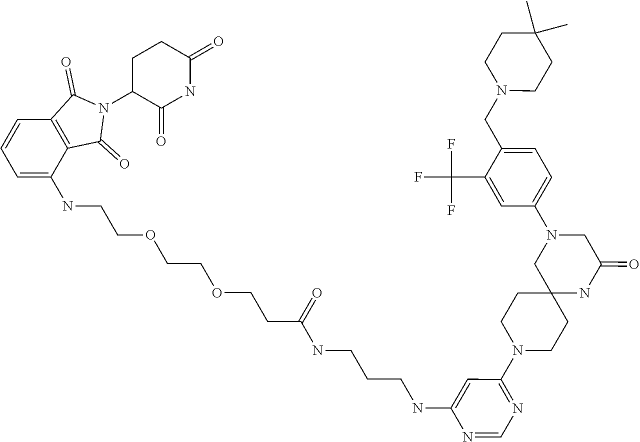 [SMILES: CC1(C)CCN(Cc2ccc(N3CC(=O)NC4(CCN(c5cc(NCCCNC(=O)CCOCCOCCNc6cccc7c6C(=O)N(C6CCC(=O)NC6=O)C7=O)ncn5)CC4)C3)cc2C(F)(F)F)CC1] |
| 54 | 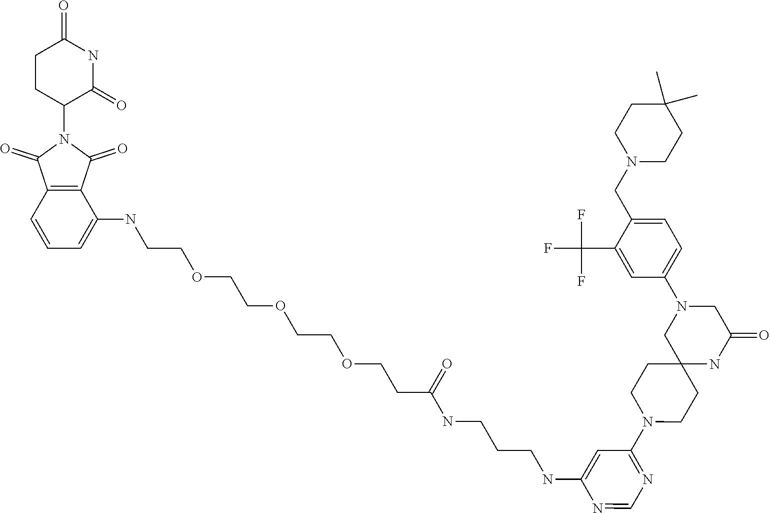 [SMILES: CC1(C)CCN(Cc2ccc(N3CC(=O)NC4(CCN(c5cc(NCCCNC(=O)CCOCCOCCOCCNc6cccc7c6C(=O)N(C6CCC(=O)NC6=O)C7=O)ncn5)CC4)C3)cc2C(F)(F)F)CC1] |
| 55 | 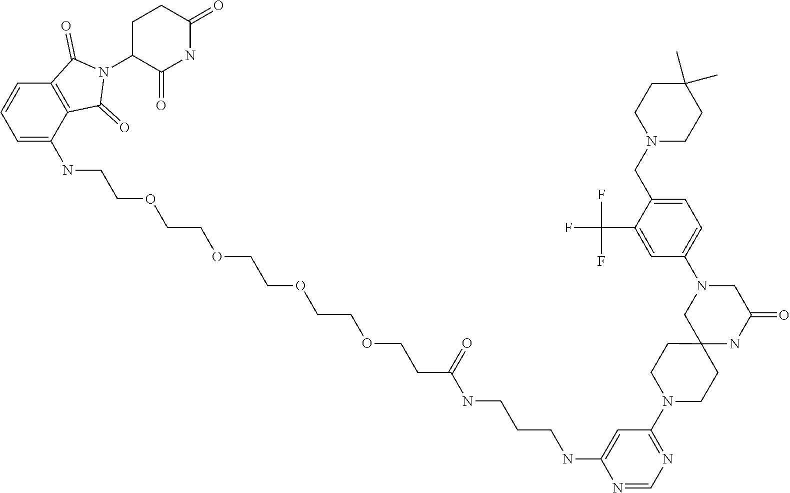 [SMILES: CC1(C)CCN(Cc2ccc(N3CC(=O)NC4(CCN(c5cc(NCCCNC(=O)CCOCCOCCOCCOCCNc6cccc7c6C(=O)N(C6CCC(=O)NC6=O)C7=O)ncn5)CC4)C3)cc2C(F)(F)F)CC1] |
| 56 | 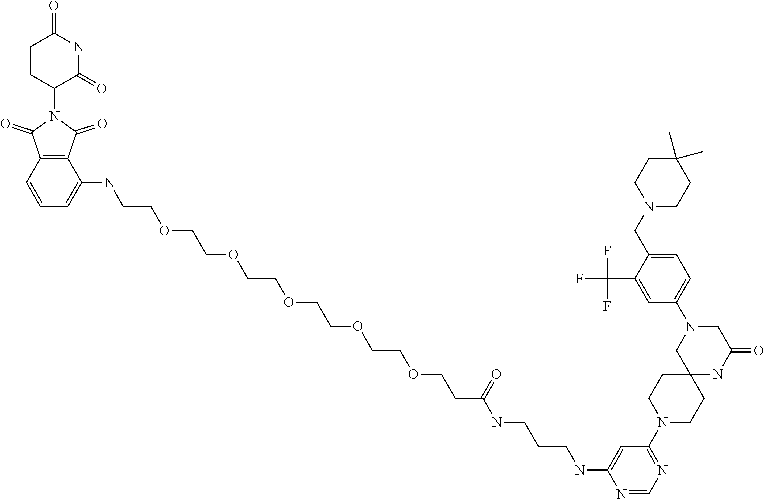 [SMILES: CC1(C)CCN(Cc2ccc(N3CC(=O)NC4(CCN(c5cc(NCCCNC(=O)CCOCCOCCOCCOCCOCCNc6cccc7c6C(=O)N(C6CCC(=O)NC6=O)C7=O)ncn5)CC4)C3)cc2C(F)(F)F)CC1] |
| 57 | 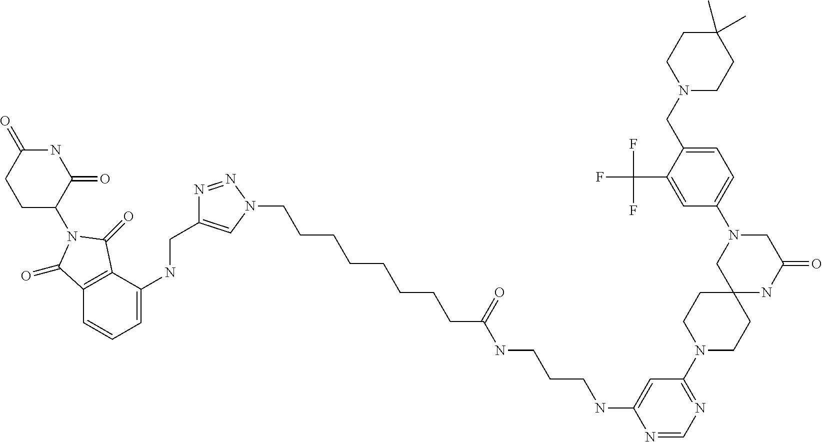 [SMILES: CC1(C)CCN(Cc2ccc(N3CC(=O)NC4(CCN(c5cc(NCCCNC(=O)CCCCCCCCn6cc(CNc7cccc8c7C(=O)N(C7CCC(=O)NC7=O)C8=O)nn6)ncn5)CC4)C3)cc2C(F)(F)F)CC1] |
| 58 | 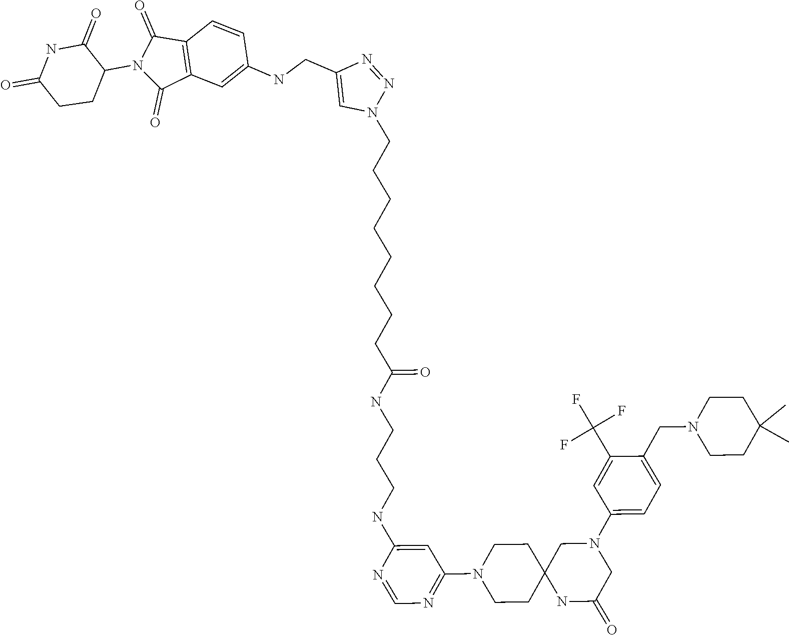 [SMILES: CC1(C)CCN(Cc2ccc(N3CC(=O)NC4(CCN(c5cc(NCCCNC(=O)CCCCCCCCn6cc(CNc7ccc8c(c7)C(=O)N(C7CCC(=O)NC7=O)C8=O)nn6)ncn5)CC4)C3)cc2C(F)(F)F)CC1] |
| 59 | 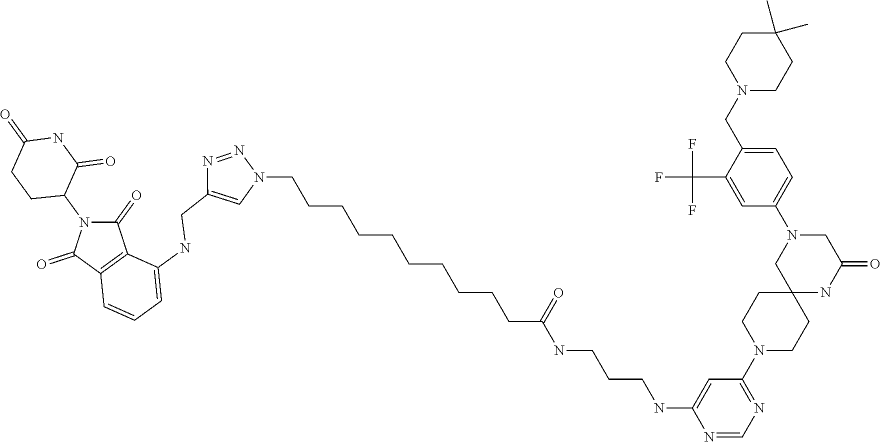 [SMILES: CC1(C)CCN(Cc2ccc(N3CC(=O)NC4(CCN(c5cc(NCCCNC(=O)CCCCCCCCCCn6cc(CNc7cccc8c7C(=O)N(C7CCC(=O)NC7=O)C8=O)nn6)ncn5)CC4)C3)cc2C(F)(F)F)CC1] |
| 60 | 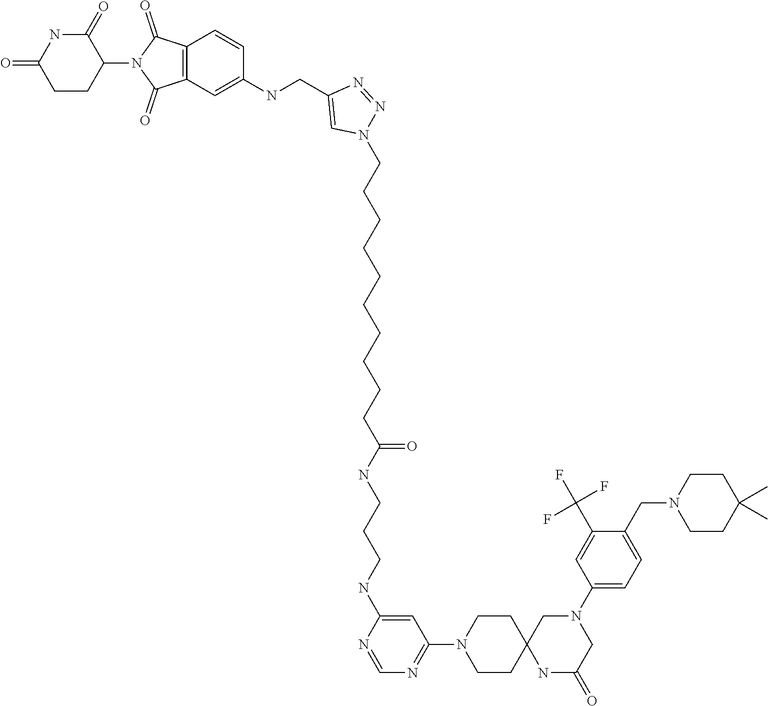 [SMILES: CC1(C)CCN(Cc2ccc(N3CC(=O)NC4(CCN(c5cc(NCCCNC(=O)CCCCCCCCCCn6cc(CNc7ccc8c(c7)C(=O)N(C7CCC(=O)NC7=O)C8=O)nn6)ncn5)CC4)C3)cc2C(F)(F)F)CC1] |
| 61 | 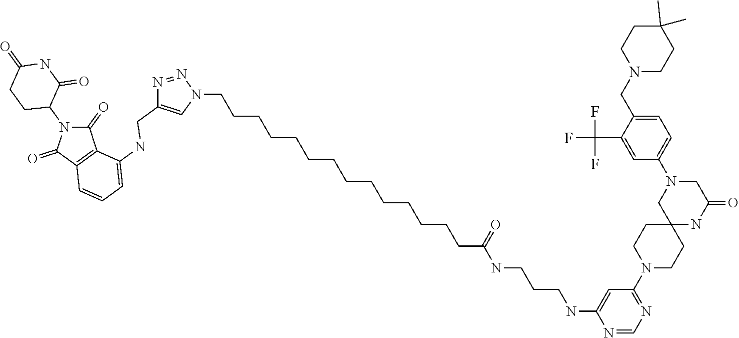 [SMILES: CC1(C)CCN(Cc2ccc(N3CC(=O)NC4(CCN(c5cc(NCCCNC(=O)CCCCCCCCCCCCCCn6cc(CNc7cccc8c7C(=O)N(C7CCC(=O)NC7=O)C8=O)nn6)ncn5)CC4)C3)cc2C(F)(F)F)CC1] |
| 62 | 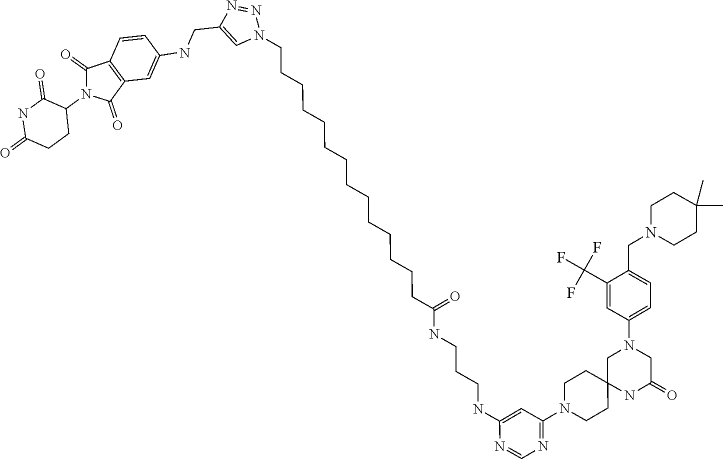 [SMILES: CC1(C)CCN(Cc2ccc(N3CC(=O)NC4(CCN(c5cc(NCCCNC(=O)CCCCCCCCCCCCCCn6cc(CNc7ccc8c(c7)C(=O)N(C7CCC(=O)NC7=O)C8=O)nn6)ncn5)CC4)C3)cc2C(F)(F)F)CC1] |
| 63 | 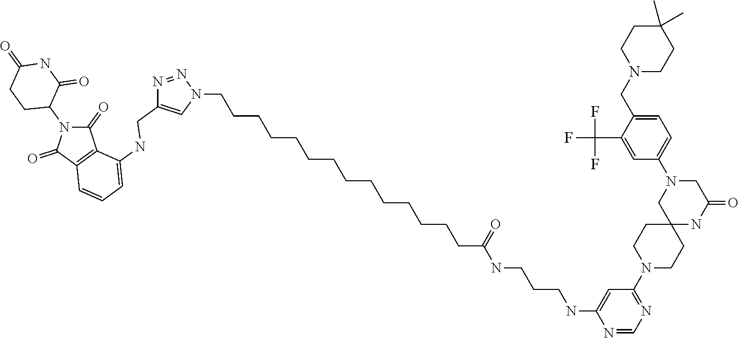 [SMILES: CC1(C)CCN(Cc2ccc(N3CC(=O)NC4(CCN(c5cc(NCCCNC(=O)CCCCCCCCCCCCCCn6cc(CNc7cccc8c7C(=O)N(C7CCC(=O)NC7=O)C8=O)nn6)ncn5)CC4)C3)cc2C(F)(F)F)CC1] |
| 64 | 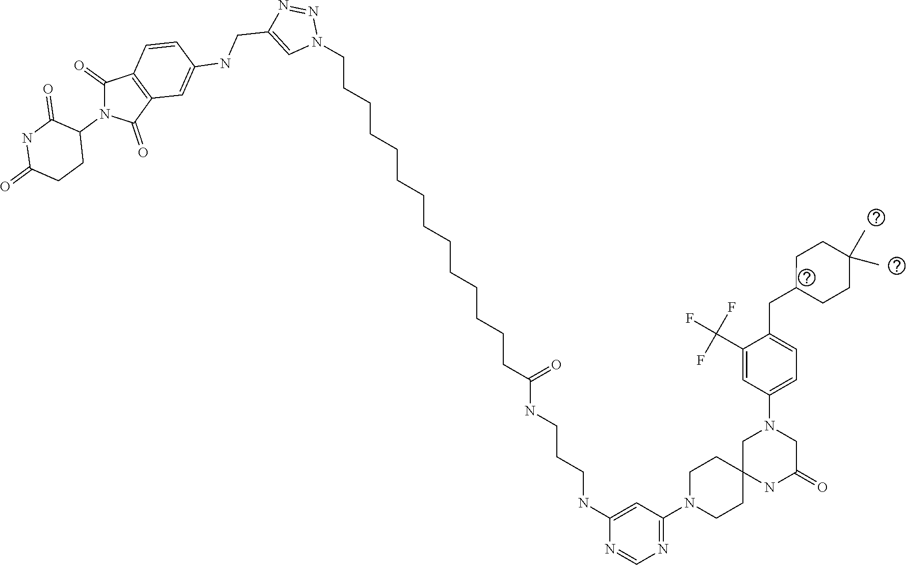 [SMILES: CC1(C)CCC(Cc2ccc(N3CC(=O)NC4(CCN(c5cc(NCCCNC(=O)CCCCCCCCCCCCCCn6cc(CNc7ccc8c(c7)C(=O)N(C7CCC(=O)NC7=O)C8=O)nn6)ncn5)CC4)C3)cc2C(F)(F)F)CC1] |
| 65 | 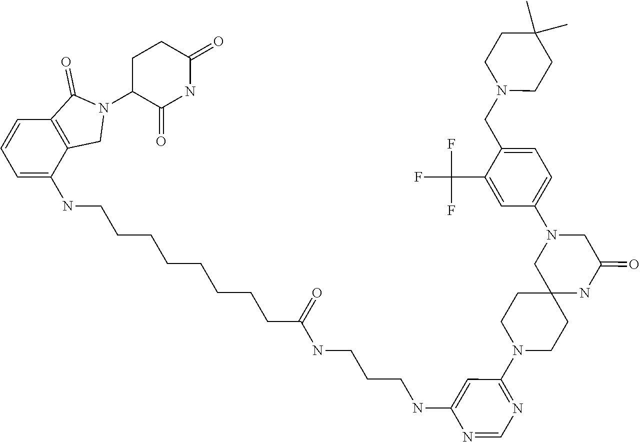 [SMILES: CC1(C)CCN(Cc2ccc(N3CC(=O)NC4(CCN(c5cc(NCCCNC(=O)CCCCCCCCNc6cccc7c6CN(C6CCC(=O)NC6=O)C7=O)ncn5)CC4)C3)cc2C(F)(F)F)CC1] |
| 66 | 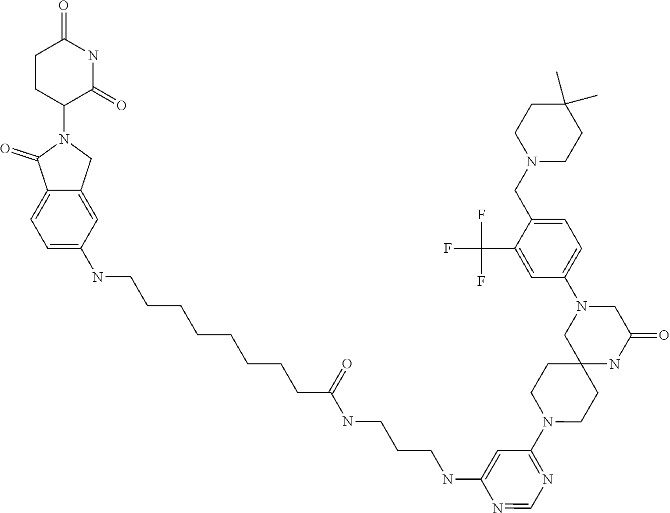 [SMILES: CC1(C)CCN(Cc2ccc(N3CC(=O)NC4(CCN(c5cc(NCCCNC(=O)CCCCCCCCNc6ccc7c(c6)CN(C6CCC(=O)NC6=O)C7=O)ncn5)CC4)C3)cc2C(F)(F)F)CC1] |
| 67 | 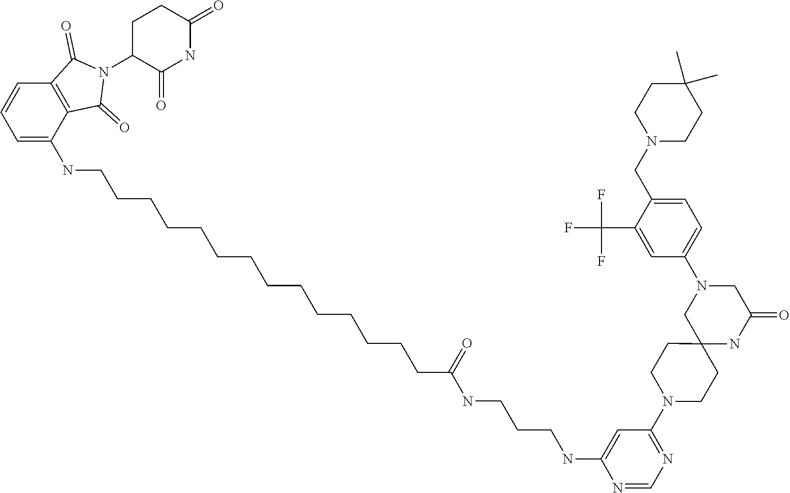 [SMILES: CC1(C)CCN(Cc2ccc(N3CC(=O)NC4(CCN(c5cc(NCCCNC(=O)CCCCCCCCCCCCCCNc6cccc7c6C(=O)N(C6CCC(=O)NC6=O)C7=O)ncn5)CC4)C3)cc2C(F)(F)F)CC1] |
| 68 | 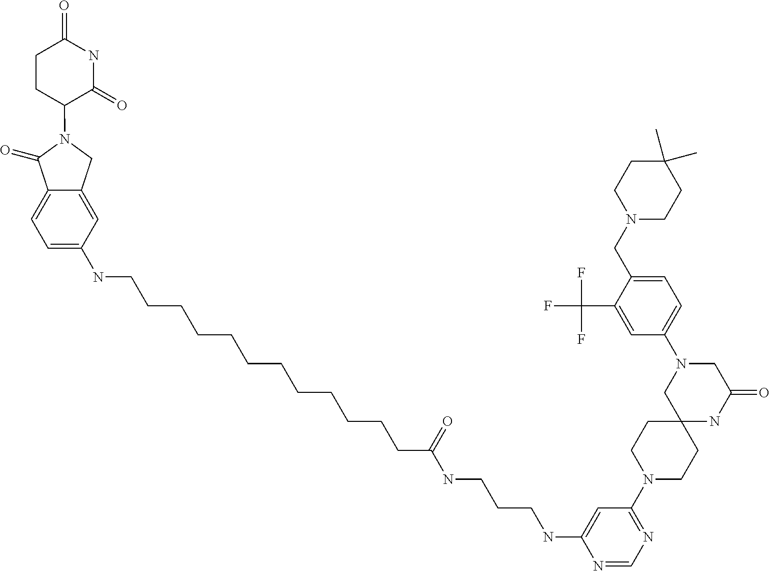 [SMILES: CC1(C)CCN(Cc2ccc(N3CC(=O)NC4(CCN(c5cc(NCCCNC(=O)CCCCCCCCCCCCNc6ccc7c(c6)CN(C6CCC(=O)NC6=O)C7=O)ncn5)CC4)C3)cc2C(F)(F)F)CC1] |
| 69 | 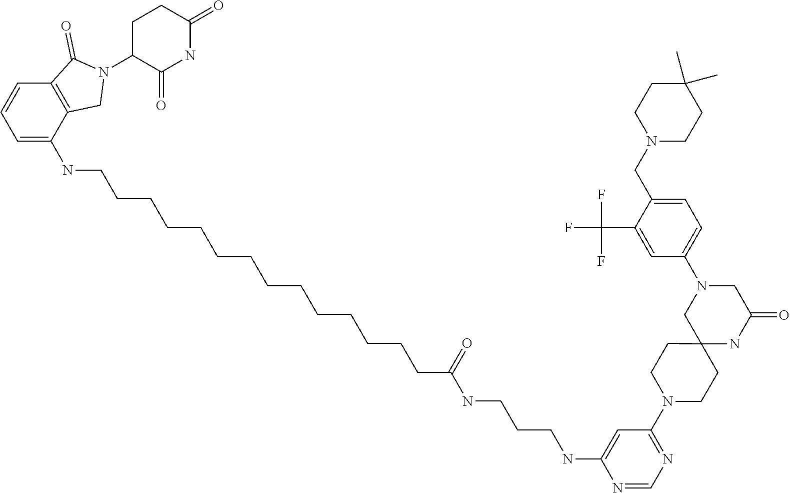 [SMILES: CC1(C)CCN(Cc2ccc(N3CC(=O)NC4(CCN(c5cc(NCCCNC(=O)CCCCCCCCCCCCCCNc6cccc7c6CN(C6CCC(=O)NC6=O)C7=O)ncn5)CC4)C3)cc2C(F)(F)F)CC1] |
| 70 | 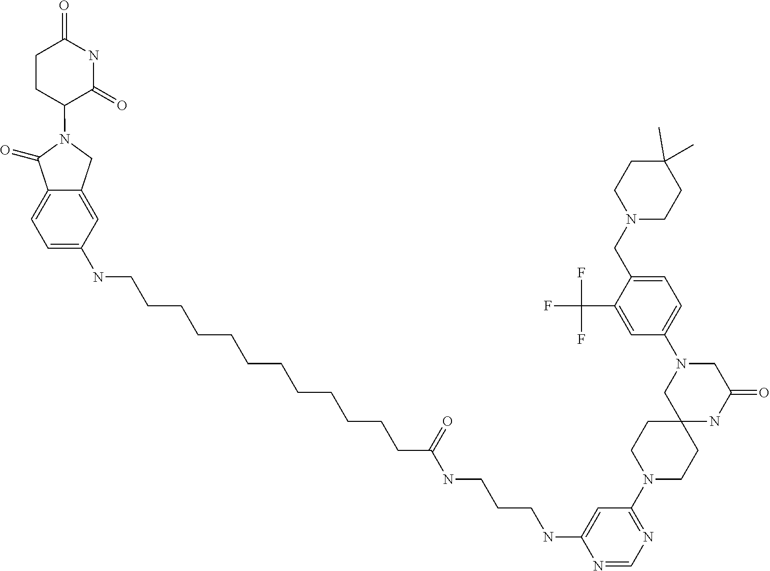 [SMILES: CC1(C)CCN(Cc2ccc(N3CC(=O)NC4(CCN(c5cc(NCCCNC(=O)CCCCCCCCCCCCNc6ccc7c(c6)CN(C6CCC(=O)NC6=O)C7=O)ncn5)CC4)C3)cc2C(F)(F)F)CC1] |
| 71 | 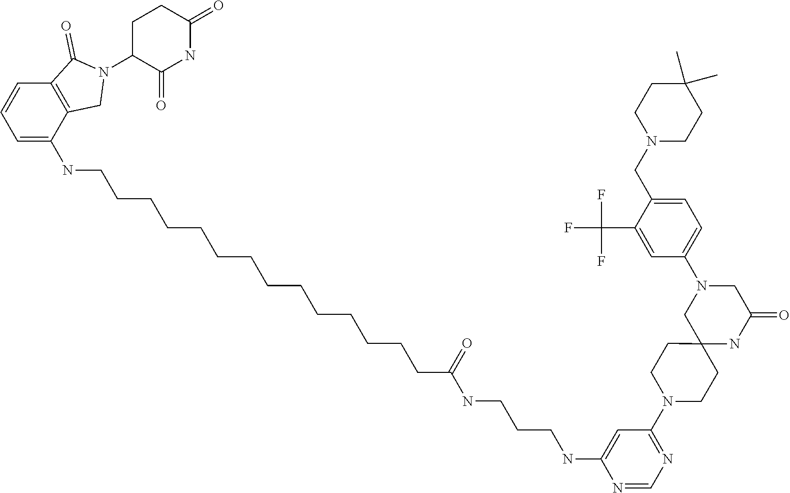 [SMILES: CC1(C)CCN(Cc2ccc(N3CC(=O)NC4(CCN(c5cc(NCCCNC(=O)CCCCCCCCCCCCCCNc6cccc7c6CN(C6CCC(=O)NC6=O)C7=O)ncn5)CC4)C3)cc2C(F)(F)F)CC1] |
| 72 | 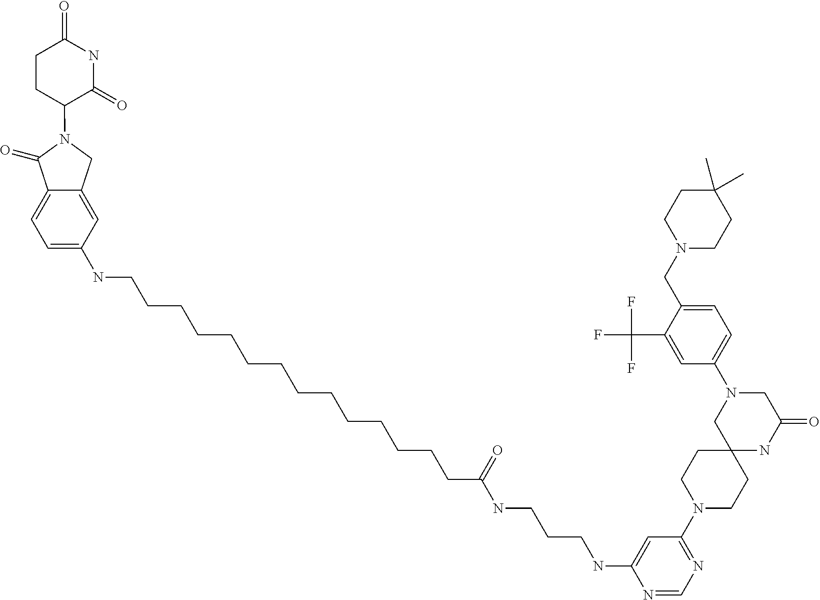 [SMILES: CC1(C)CCN(Cc2ccc(N3CC(=O)NC4(CCN(c5cc(NCCCNC(=O)CCCCCCCCCCCCCCNc6ccc7c(c6)CN(C6CCC(=O)NC6=O)C7=O)ncn5)CC4)C3)cc2C(F)(F)F)CC1] |
| 73 | 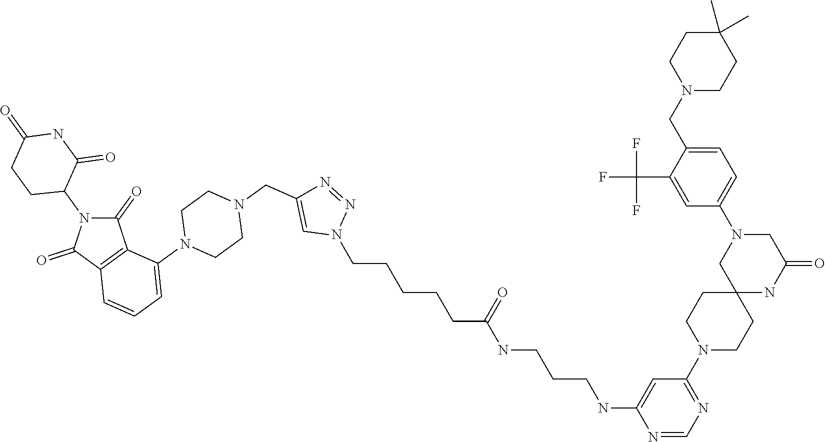 [SMILES: CC1(C)CCN(Cc2ccc(N3CC(=O)NC4(CCN(c5cc(NCCCNC(=O)CCCCCn6cc(CN7CCN(c8cccc9c8C(=O)N(C8CCC(=O)NC8=O)C9=O)CC7)nn6)ncn5)CC4)C3)cc2C(F)(F)F)CC1] |
| 74 | 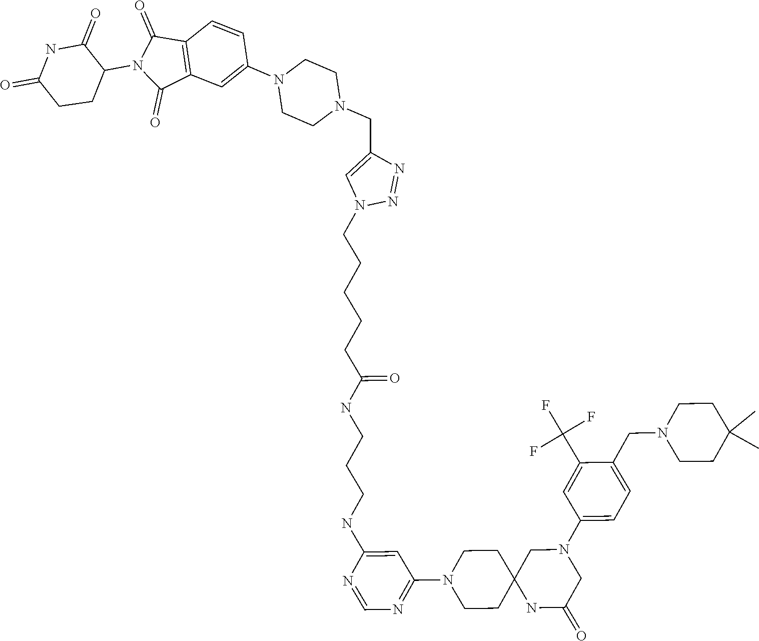 [SMILES: CC1(C)CCN(Cc2ccc(N3CC(=O)NC4(CCN(c5cc(NCCCNC(=O)CCCCCn6cc(CN7CCN(c8ccc9c(c8)C(=O)N(C8CCC(=O)NC8=O)C9=O)CC7)nn6)ncn5)CC4)C3)cc2C(F)(F)F)CC1] |
| 75 | 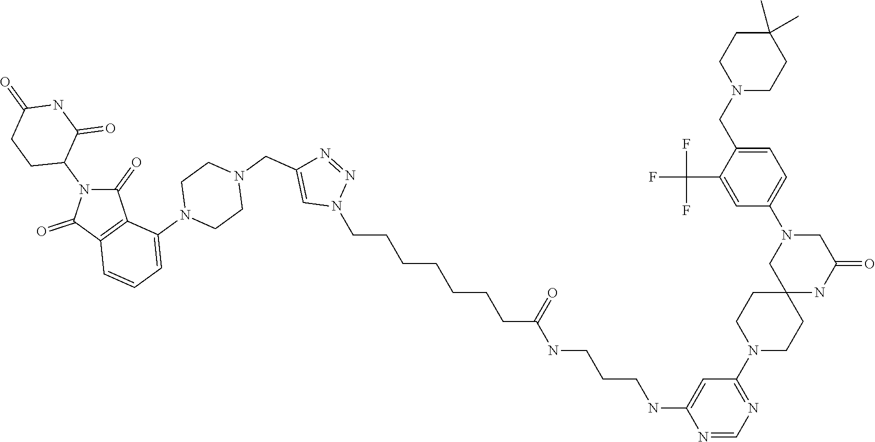 [SMILES: CC1(C)CCN(Cc2ccc(N3CC(=O)NC4(CCN(c5cc(NCCCNC(=O)CCCCCCCn6cc(CN7CCN(c8cccc9c8C(=O)N(C8CCC(=O)NC8=O)C9=O)CC7)nn6)ncn5)CC4)C3)cc2C(F)(F)F)CC1] |
| 76 | 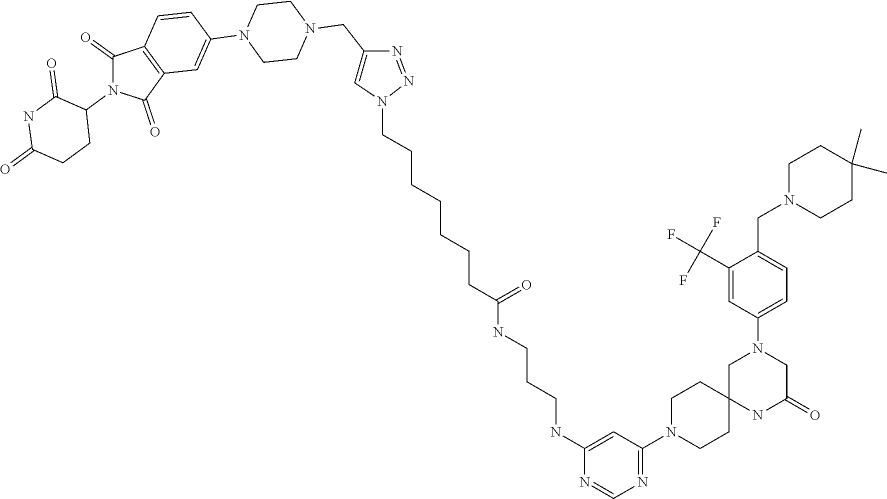 [SMILES: CC1(C)CCN(Cc2ccc(N3CC(=O)NC4(CCN(c5cc(NCCCNC(=O)CCCCCCCn6cc(CN7CCN(c8ccc9c(c8)C(=O)N(C8CCC(=O)NC8=O)C9=O)CC7)nn6)ncn5)CC4)C3)cc2C(F)(F)F)CC1] |
| 77 | 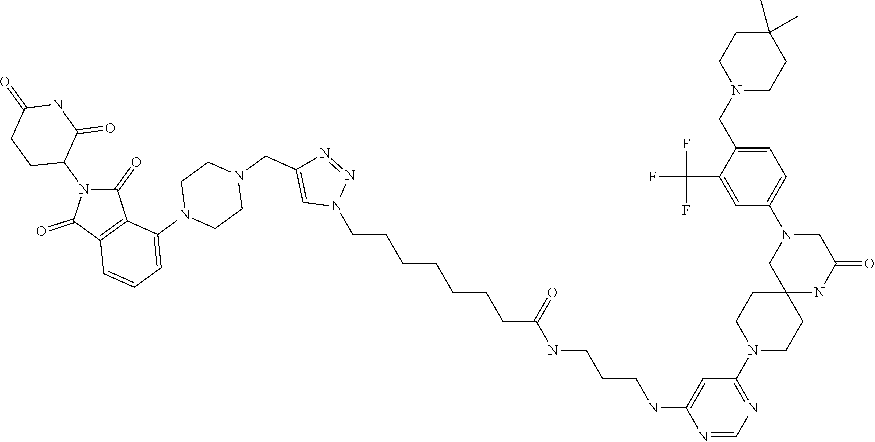 [SMILES: CC1(C)CCN(Cc2ccc(N3CC(=O)NC4(CCN(c5cc(NCCCNC(=O)CCCCCCCn6cc(CN7CCN(c8cccc9c8C(=O)N(C8CCC(=O)NC8=O)C9=O)CC7)nn6)ncn5)CC4)C3)cc2C(F)(F)F)CC1] |
| 78 | 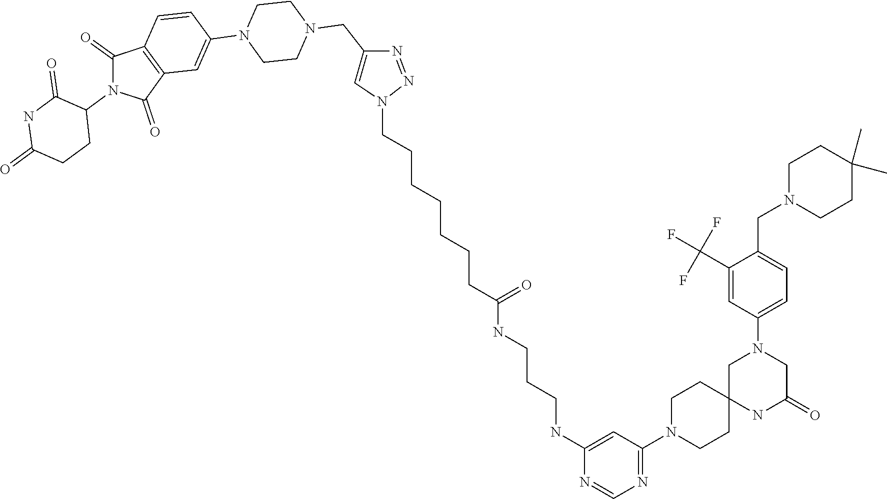 [SMILES: CC1(C)CCN(Cc2ccc(N3CC(=O)NC4(CCN(c5cc(NCCCNC(=O)CCCCCCCn6cc(CN7CCN(c8ccc9c(c8)C(=O)N(C8CCC(=O)NC8=O)C9=O)CC7)nn6)ncn5)CC4)C3)cc2C(F)(F)F)CC1] |
| 79 | 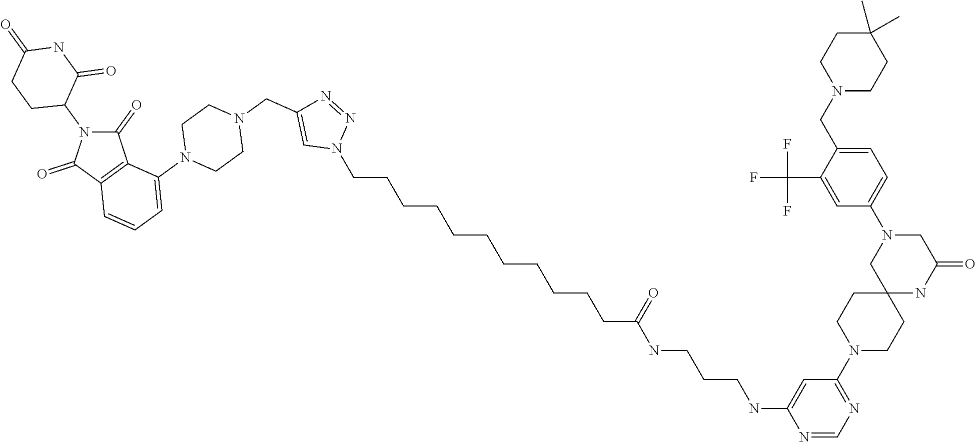 [SMILES: CC1(C)CCN(Cc2ccc(N3CC(=O)NC4(CCN(c5cc(NCCCNC(=O)CCCCCCCCCCCn6cc(CN7CCN(c8cccc9c8C(=O)N(C8CCC(=O)NC8=O)C9=O)CC7)nn6)ncn5)CC4)C3)cc2C(F)(F)F)CC1] |
| 80 | 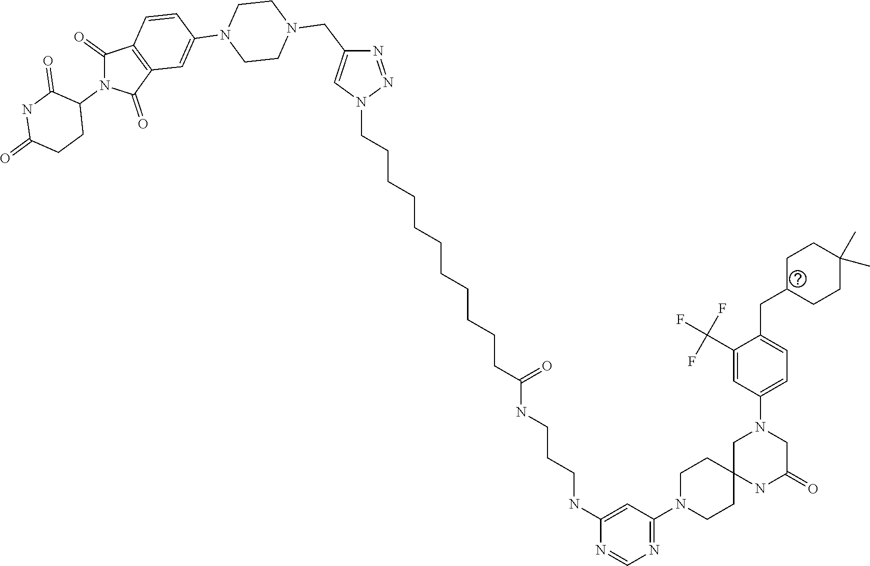 [SMILES: CC1(C)CCC(Cc2ccc(N3CC(=O)NC4(CCN(c5cc(NCCCNC(=O)CCCCCCCCCCCn6cc(CN7CCN(c8ccc9c(c8)C(=O)N(C8CCC(=O)NC8=O)C9=O)CC7)nn6)ncn5)CC4)C3)cc2C(F)(F)F)CC1] |
| 81 | 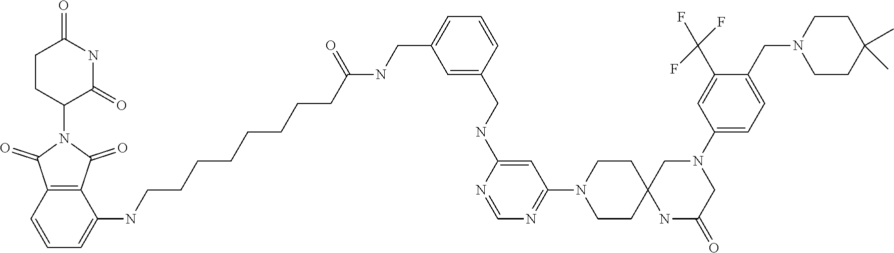 [SMILES: CC1(C)CCN(Cc2ccc(N3CC(=O)NC4(CCN(c5cc(NCc6cccc(CNC(=O)CCCCCCCCNc7cccc8c7C(=O)N(C7CCC(=O)NC7=O)C8=O)c6)ncn5)CC4)C3)cc2C(F)(F)F)CC1] |
| 82 | 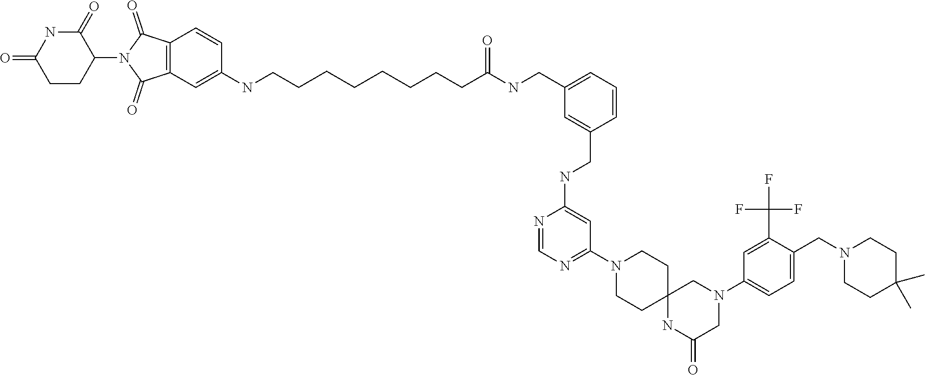 [SMILES: CC1(C)CCN(Cc2ccc(N3CC(=O)NC4(CCN(c5cc(NCc6cccc(CNC(=O)CCCCCCCCNc7ccc8c(c7)C(=O)N(C7CCC(=O)NC7=O)C8=O)c6)ncn5)CC4)C3)cc2C(F)(F)F)CC1] |
| 83 | 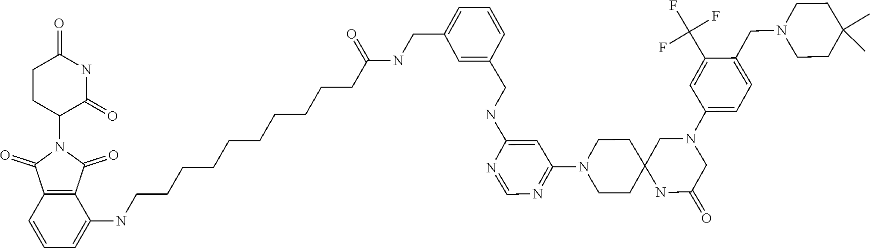 [SMILES: CC1(C)CCN(Cc2ccc(N3CC(=O)NC4(CCN(c5cc(NCc6cccc(CNC(=O)CCCCCCCCCCNc7cccc8c7C(=O)N(C7CCC(=O)NC7=O)C8=O)c6)ncn5)CC4)C3)cc2C(F)(F)F)CC1] |
| 84 | 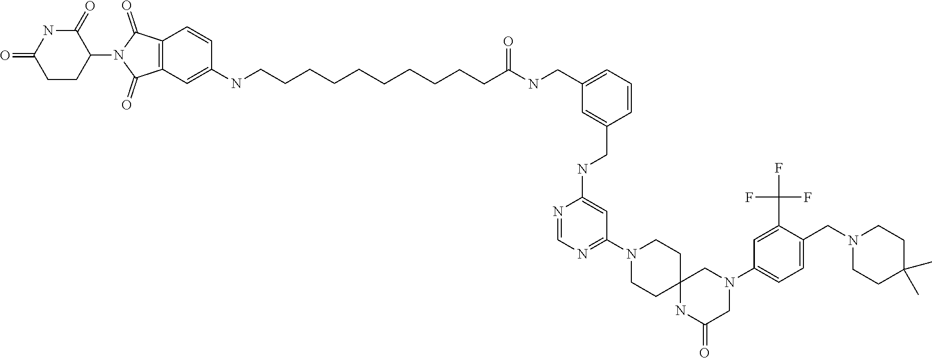 [SMILES: CC1(C)CCN(Cc2ccc(N3CC(=O)NC4(CCN(c5cc(NCc6cccc(CNC(=O)CCCCCCCCCCNc7ccc8c(c7)C(=O)N(C7CCC(=O)NC7=O)C8=O)c6)ncn5)CC4)C3)cc2C(F)(F)F)CC1] |
| 85 | 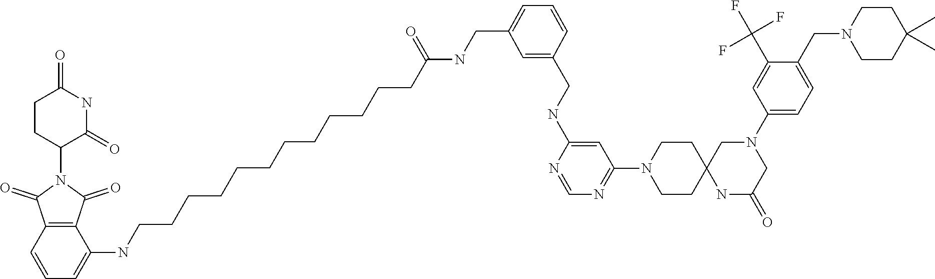 [SMILES: CC1(C)CCN(Cc2ccc(N3CC(=O)NC4(CCN(c5cc(NCc6cccc(CNC(=O)CCCCCCCCCCCCNc7cccc8c7C(=O)N(C7CCC(=O)NC7=O)C8=O)c6)ncn5)CC4)C3)cc2C(F)(F)F)CC1] |
| 86 | 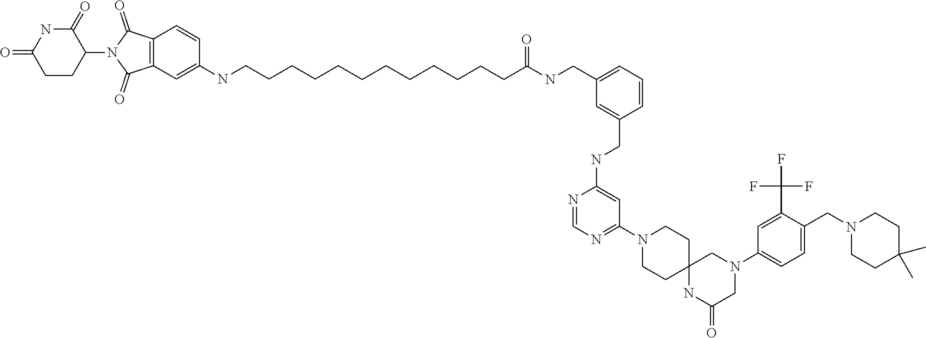 [SMILES: CC1(C)CCN(Cc2ccc(N3CC(=O)NC4(CCN(c5cc(NCc6cccc(CNC(=O)CCCCCCCCCCCCNc7ccc8c(c7)C(=O)N(C7CCC(=O)NC7=O)C8=O)c6)ncn5)CC4)C3)cc2C(F)(F)F)CC1] |
| 87 | 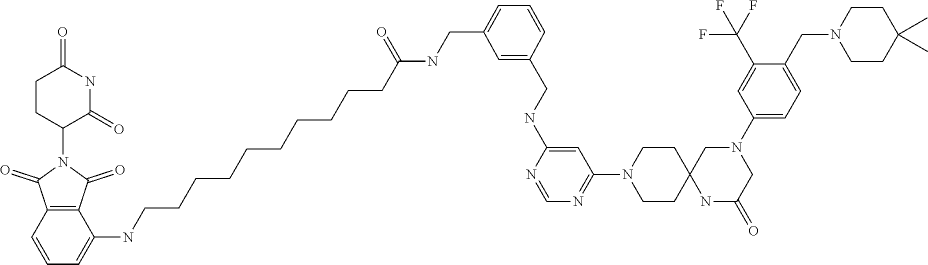 [SMILES: CC1(C)CCN(Cc2ccc(N3CC(=O)NC4(CCN(c5cc(NCc6cccc(CNC(=O)CCCCCCCCCCNc7cccc8c7C(=O)N(C7CCC(=O)NC7=O)C8=O)c6)ncn5)CC4)C3)cc2C(F)(F)F)CC1] |
| 88 | 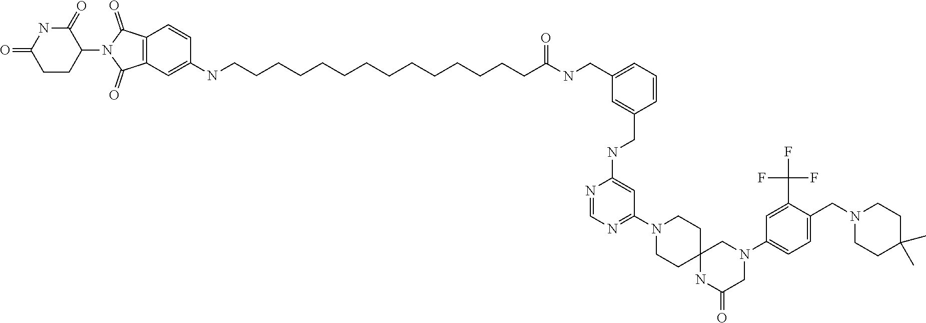 [SMILES: CC1(C)CCN(Cc2ccc(N3CC(=O)NC4(CCN(c5cc(NCc6cccc(CNC(=O)CCCCCCCCCCCCCCNc7ccc8c(c7)C(=O)N(C7CCC(=O)NC7=O)C8=O)c6)ncn5)CC4)C3)cc2C(F)(F)F)CC1] |
| 89 | 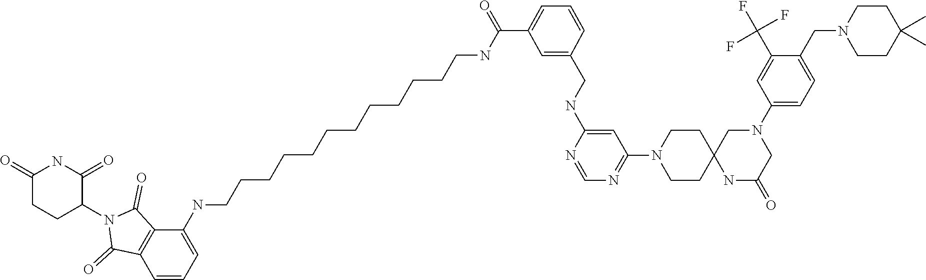 [SMILES: CC1(C)CCN(Cc2ccc(N3CC(=O)NC4(CCN(c5cc(NCc6cccc(C(=O)NCCCCCCCCCCCCNc7cccc8c7C(=O)N(C7CCC(=O)NC7=O)C8=O)c6)ncn5)CC4)C3)cc2C(F)(F)F)CC1] |
| 90 | 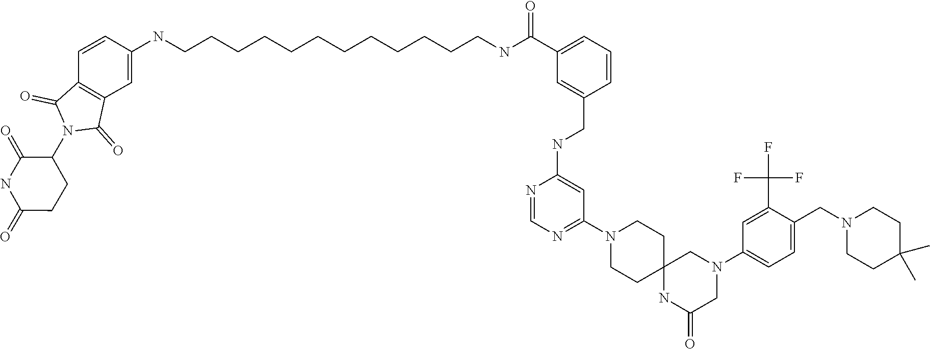 [SMILES: CC1(C)CCN(Cc2ccc(N3CC(=O)NC4(CCN(c5cc(NCc6cccc(C(=O)NCCCCCCCCCCCCNc7ccc8c(c7)C(=O)N(C7CCC(=O)NC7=O)C8=O)c6)ncn5)CC4)C3)cc2C(F)(F)F)CC1] |
| 91 | 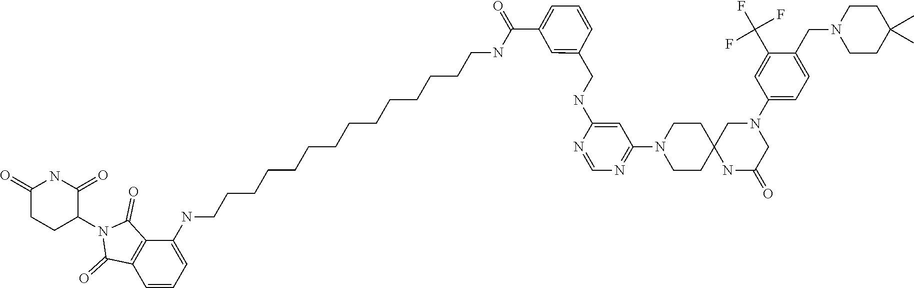 [SMILES: CC1(C)CCN(Cc2ccc(N3CC(=O)NC4(CCN(c5cc(NCc6cccc(C(=O)NCCCCCCCCCCCCCCNc7cccc8c7C(=O)N(C7CCC(=O)NC7=O)C8=O)c6)ncn5)CC4)C3)cc2C(F)(F)F)CC1] |
| 92 | 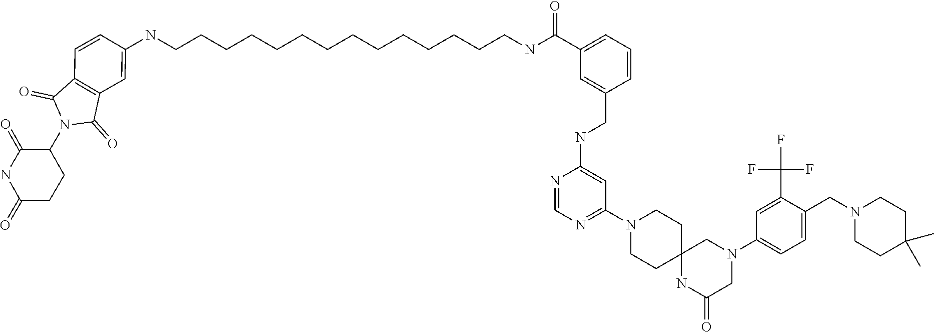 [SMILES: CC1(C)CCN(Cc2ccc(N3CC(=O)NC4(CCN(c5cc(NCc6cccc(C(=O)NCCCCCCCCCCCCCCNc7ccc8c(c7)C(=O)N(C7CCC(=O)NC7=O)C8=O)c6)ncn5)CC4)C3)cc2C(F)(F)F)CC1] |
| 93 | 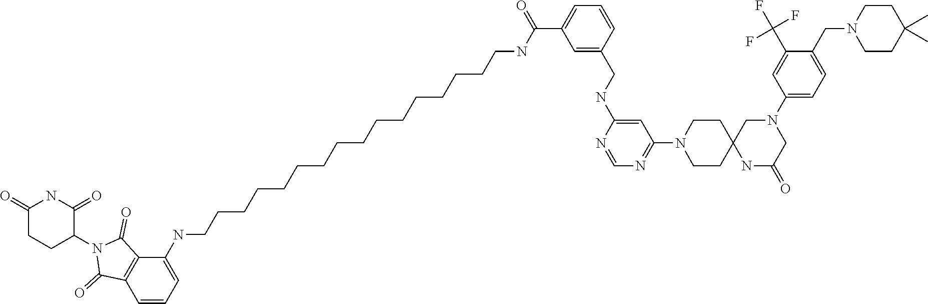 [SMILES: CC1(C)CCN(Cc2ccc(N3CC(=O)NC4(CCN(c5cc(NCc6cccc(C(=O)NCCCCCCCCCCCCCCCCNc7cccc8c7C(=O)N(C7CCC(=O)NC7=O)C8=O)c6)ncn5)CC4)C3)cc2C(F)(F)F)CC1] |
| 94 | 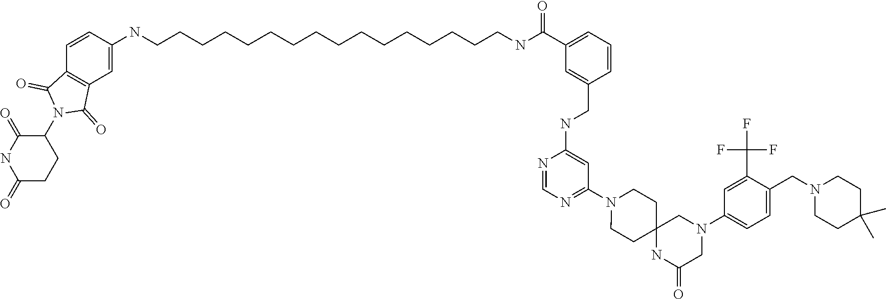 [SMILES: CC1(C)CCN(Cc2ccc(N3CC(=O)NC4(CCN(c5cc(NCc6cccc(C(=O)NCCCCCCCCCCCCCCCCNc7ccc8c(c7)C(=O)N(C7CCC(=O)NC7=O)C8=O)c6)ncn5)CC4)C3)cc2C(F)(F)F)CC1] |
| 95 | 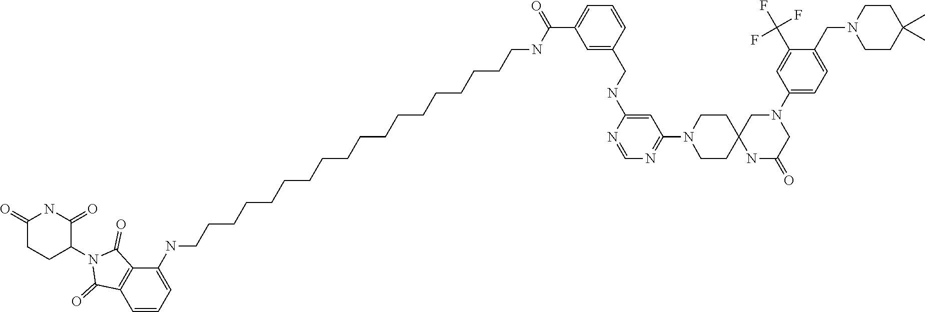 [SMILES: CC1(C)CCN(Cc2ccc(N3CC(=O)NC4(CCN(c5cc(NCc6cccc(C(=O)NCCCCCCCCCCCCCCCCCCNc7cccc8c7C(=O)N(C7CCC(=O)NC7=O)C8=O)c6)ncn5)CC4)C3)cc2C(F)(F)F)CC1] |
| 96 | 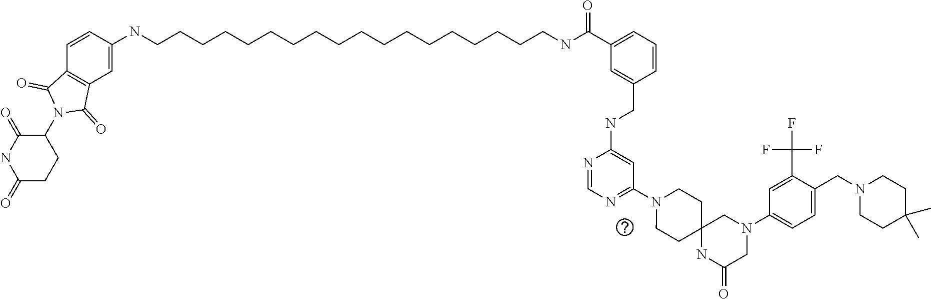 [SMILES: CC1(C)CCN(Cc2ccc(N3CC(=O)NC4(CCN(c5cc(NCc6cccc(C(=O)NCCCCCCCCCCCCCCCCCCNc7ccc8c(c7)C(=O)N(C7CCC(=O)NC7=O)C8=O)c6)ncn5)CC4)C3)cc2C(F)(F)F)CC1] |
| 97 | 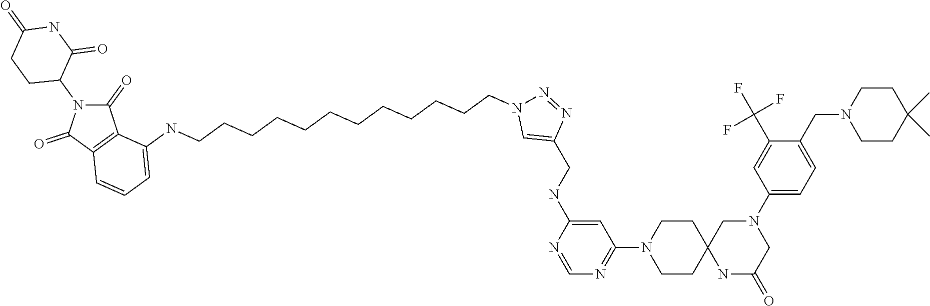 [SMILES: CC1(C)CCN(Cc2ccc(N3CC(=O)NC4(CCN(c5cc(NCc6cn(CCCCCCCCCCCCNc7cccc8c7C(=O)N(C7CCC(=O)NC7=O)C8=O)nn6)ncn5)CC4)C3)cc2C(F)(F)F)CC1] |
| 98 | 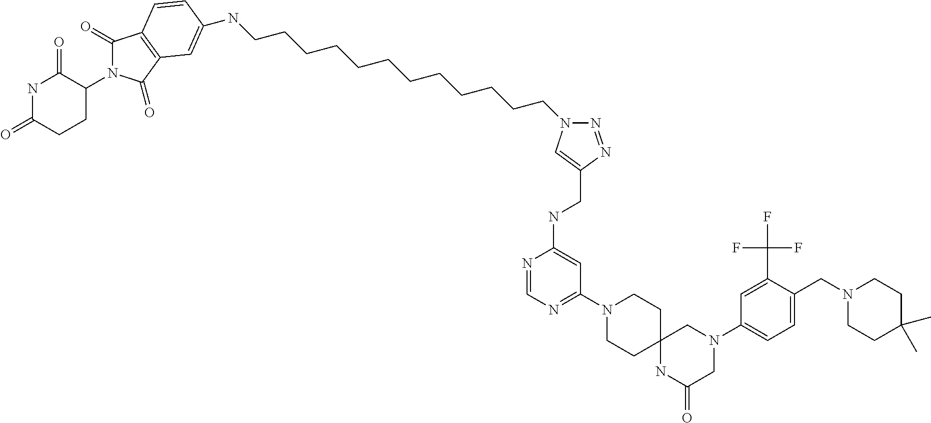 [SMILES: CC1(C)CCN(Cc2ccc(N3CC(=O)NC4(CCN(c5cc(NCc6cn(CCCCCCCCCCCCNc7ccc8c(c7)C(=O)N(C7CCC(=O)NC7=O)C8=O)nn6)ncn5)CC4)C3)cc2C(F)(F)F)CC1] |
| 99 | 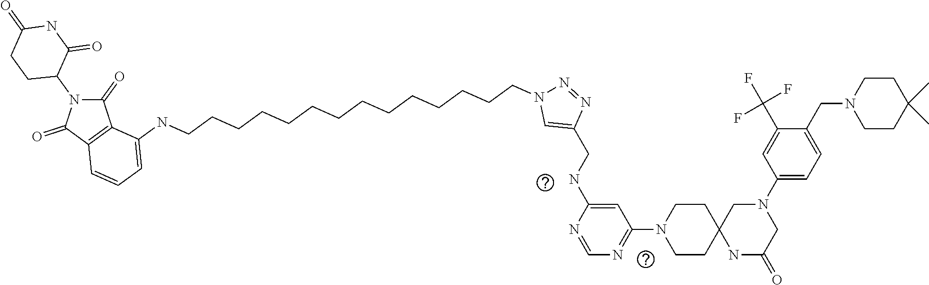 [SMILES: CC1(C)CCN(Cc2ccc(N3CC(=O)NC4(CCN(c5cc(NCc6cn(CCCCCCCCCCCCCCNc7cccc8c7C(=O)N(C7CCC(=O)NC7=O)C8=O)nn6)ncn5)CC4)C3)cc2C(F)(F)F)CC1] |
| 100 | 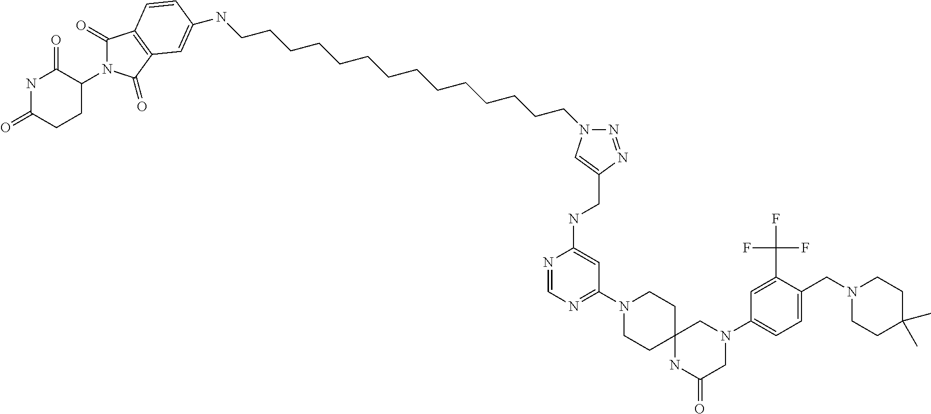 [SMILES: CC1(C)CCN(Cc2ccc(N3CC(=O)NC4(CCN(c5cc(NCc6cn(CCCCCCCCCCCCCCNc7ccc8c(c7)C(=O)N(C7CCC(=O)NC7=O)C8=O)nn6)ncn5)CC4)C3)cc2C(F)(F)F)CC1] |
| 101 | 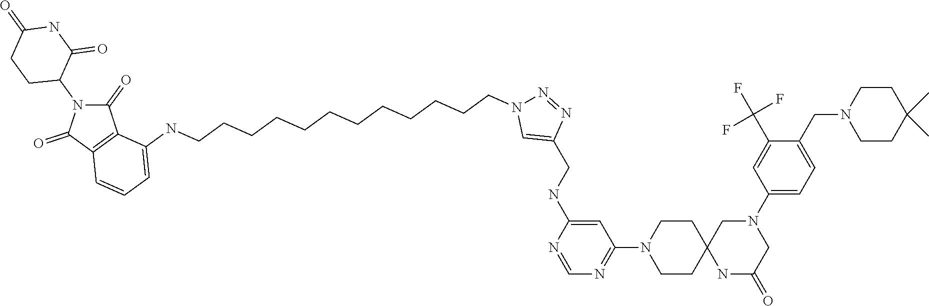 [SMILES: CC1(C)CCN(Cc2ccc(N3CC(=O)NC4(CCN(c5cc(NCc6cn(CCCCCCCCCCCCNc7cccc8c7C(=O)N(C7CCC(=O)NC7=O)C8=O)nn6)ncn5)CC4)C3)cc2C(F)(F)F)CC1] |
| 102 | 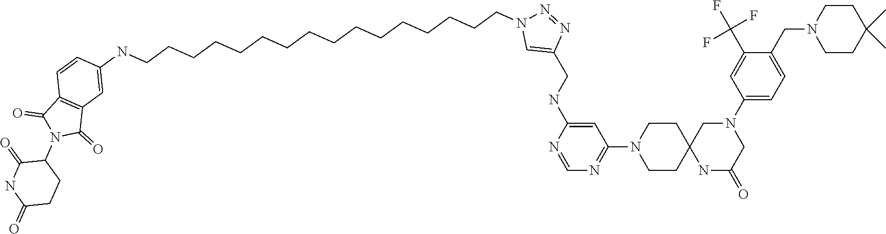 [SMILES: CC1(C)CCN(Cc2ccc(N3CC(=O)NC4(CCN(c5cc(NCc6cn(CCCCCCCCCCCCCCCCNc7ccc8c(c7)C(=O)N(C7CCC(=O)NC7=O)C8=O)nn6)ncn5)CC4)C3)cc2C(F)(F)F)CC1] |
| 103 | 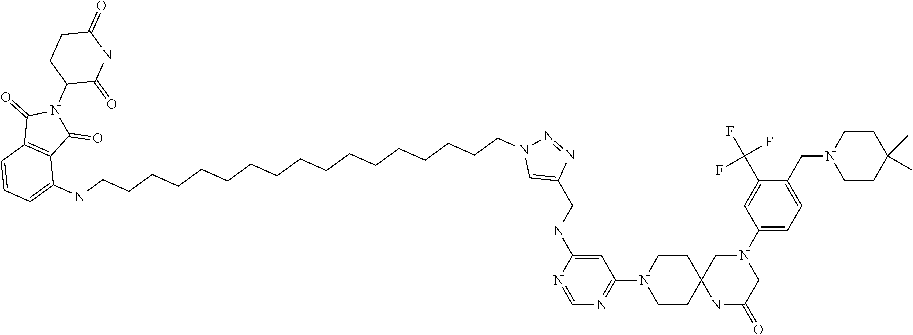 [SMILES: CC1(C)CCN(Cc2ccc(N3CC(=O)NC4(CCN(c5cc(NCc6cn(CCCCCCCCCCCCCCCCCNc7cccc8c7C(=O)N(C7CCC(=O)NC7=O)C8=O)nn6)ncn5)CC4)C3)cc2C(F)(F)F)CC1] |
| 104 | 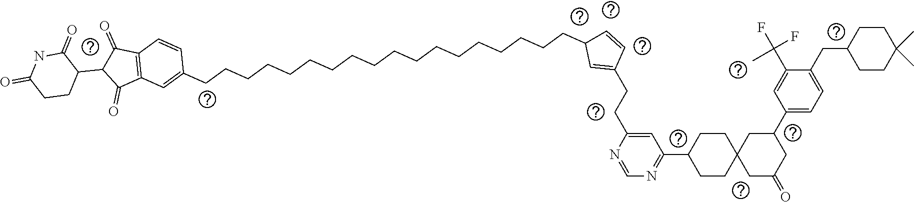 [SMILES: CC1(C)CCC(Cc2ccc(C3CC(=O)CC4(CCC(c5cc(CCC6=CC(CCCCCCCCCCCCCCCCCCc7ccc8c(c7)C(=O)C(C7CCC(=O)NC7=O)C8=O)C=C6)ncn5)CC4)C3)cc2C(C)(F)F)CC1] |
| 105 | 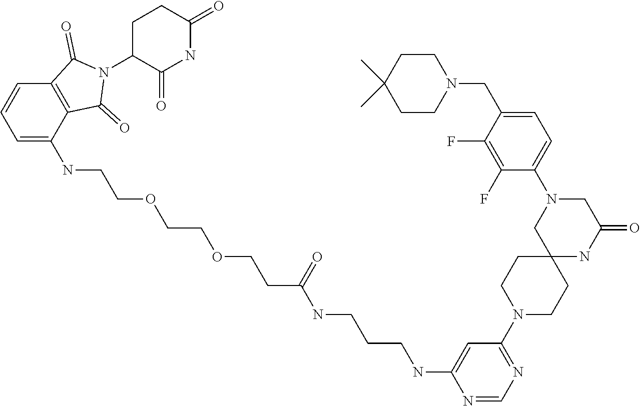 [SMILES: CC1(C)CCN(Cc2ccc(N3CC(=O)NC4(CCN(c5cc(NCCCNC(=O)CCOCCOCCNc6cccc7c6C(=O)N(C6CCC(=O)NC6=O)C7=O)ncn5)CC4)C3)c(F)c2F)CC1] |
| 106 | 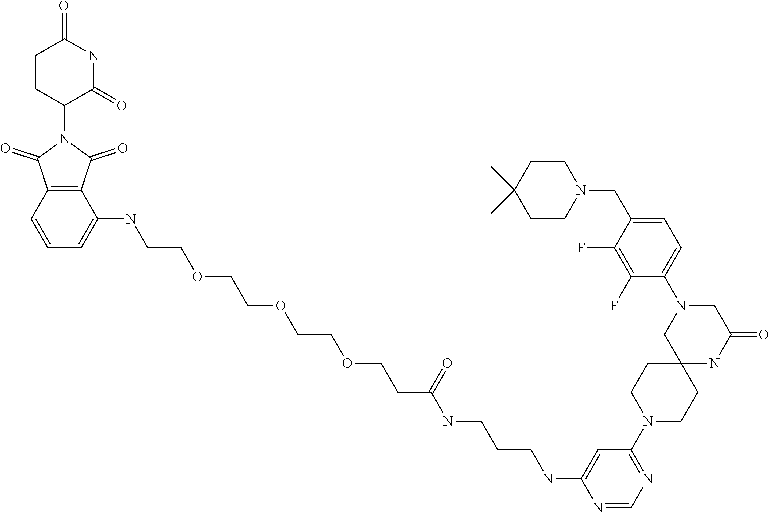 [SMILES: CC1(C)CCN(Cc2ccc(N3CC(=O)NC4(CCN(c5cc(NCCCNC(=O)CCOCCOCCOCCNc6cccc7c6C(=O)N(C6CCC(=O)NC6=O)C7=O)ncn5)CC4)C3)c(F)c2F)CC1] |
| 107 | 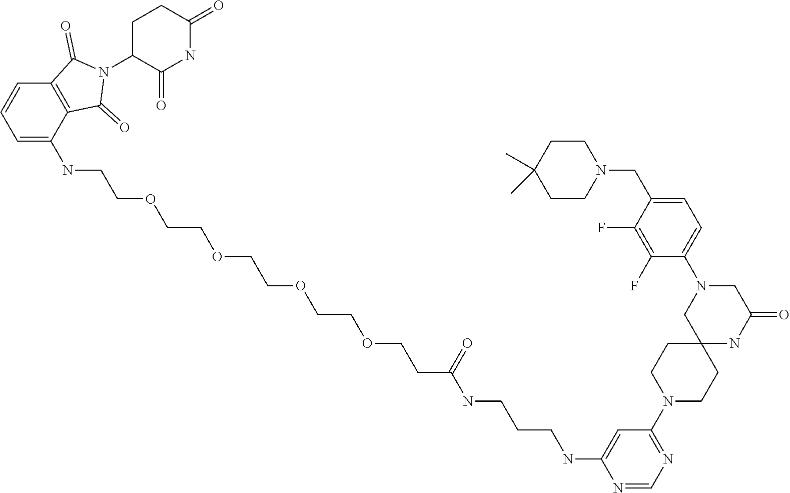 [SMILES: CC1(C)CCN(Cc2ccc(N3CC(=O)NC4(CCN(c5cc(NCCCNC(=O)CCOCCOCCOCCOCCNc6cccc7c6C(=O)N(C6CCC(=O)NC6=O)C7=O)ncn5)CC4)C3)c(F)c2F)CC1] |
| 108 | 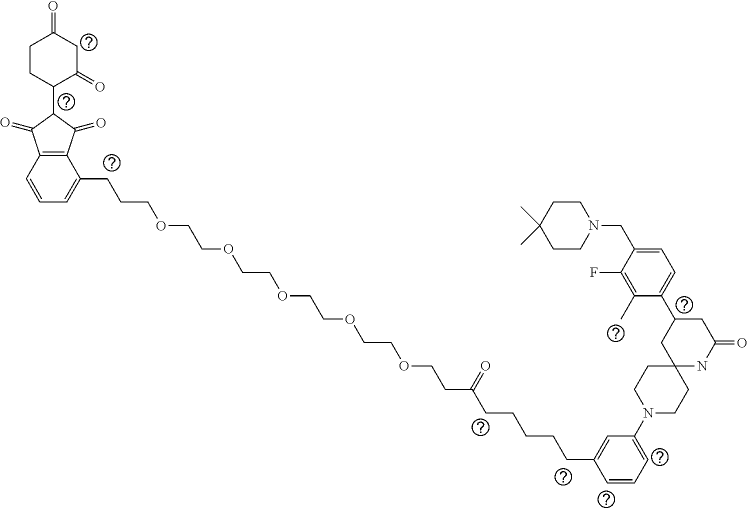 [SMILES: Cc1c(C2CC(=O)NC3(CCN(c4cccc(CCCCCC(=O)CCOCCOCCOCCOCCOCCCc5cccc6c5C(=O)C(C5CCC(=O)CC5=O)C6=O)c4)CC3)C2)ccc(CN2CCC(C)(C)CC2)c1F] |
| 109 | 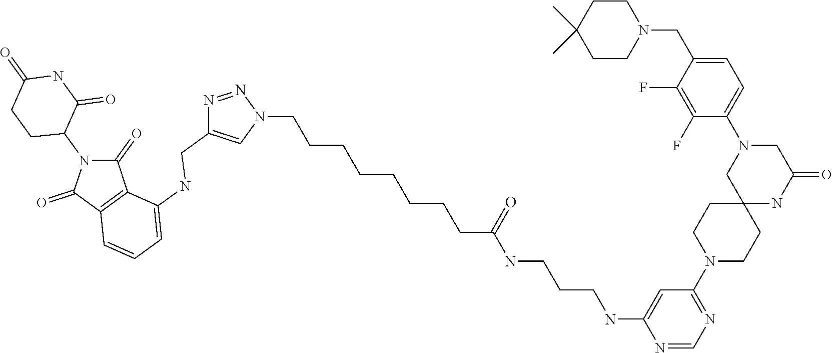 [SMILES: CC1(C)CCN(Cc2ccc(N3CC(=O)NC4(CCN(c5cc(NCCCNC(=O)CCCCCCCCn6cc(CNc7cccc8c7C(=O)N(C7CCC(=O)NC7=O)C8=O)nn6)ncn5)CC4)C3)c(F)c2F)CC1] |
| 110 | 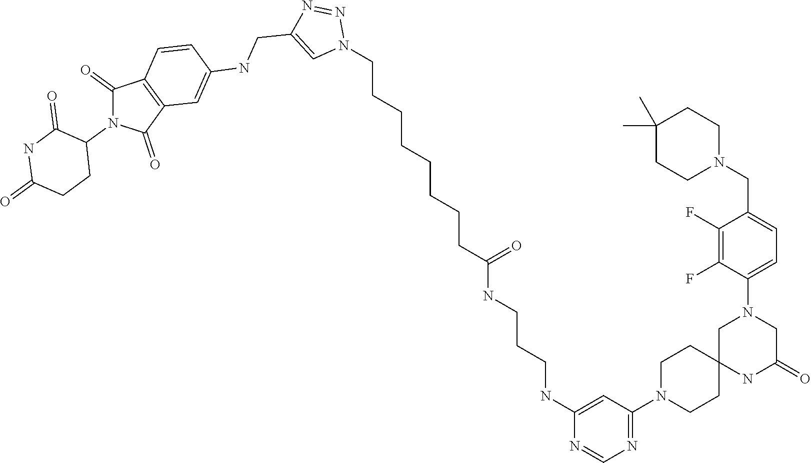 [SMILES: CC1(C)CCN(Cc2ccc(N3CC(=O)NC4(CCN(c5cc(NCCCNC(=O)CCCCCCCCn6cc(CNc7ccc8c(c7)C(=O)N(C7CCC(=O)NC7=O)C8=O)nn6)ncn5)CC4)C3)c(F)c2F)CC1] |
| 111 | 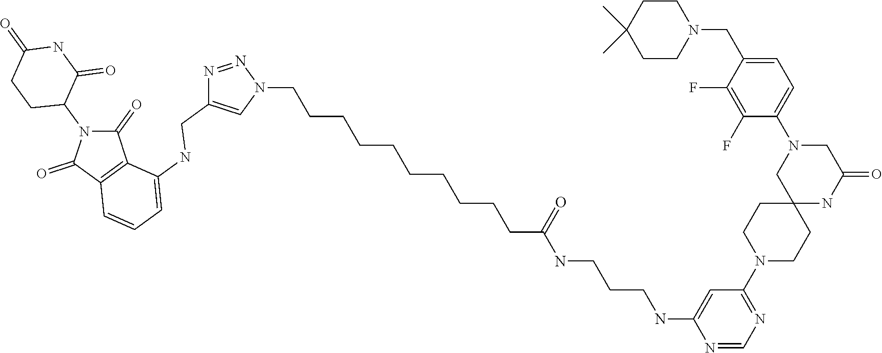 [SMILES: CC1(C)CCN(Cc2ccc(N3CC(=O)NC4(CCN(c5cc(NCCCNC(=O)CCCCCCCCCCn6cc(CNc7cccc8c7C(=O)N(C7CCC(=O)NC7=O)C8=O)nn6)ncn5)CC4)C3)c(F)c2F)CC1] |
| 112 | 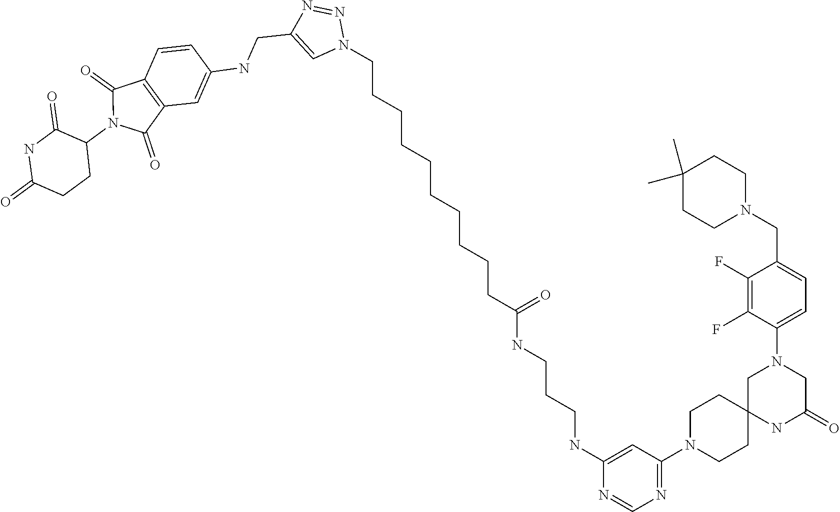 [SMILES: CC1(C)CCN(Cc2ccc(N3CC(=O)NC4(CCN(c5cc(NCCCNC(=O)CCCCCCCCCCn6cc(CNc7ccc8c(c7)C(=O)N(C7CCC(=O)NC7=O)C8=O)nn6)ncn5)CC4)C3)c(F)c2F)CC1] |
| 113 | 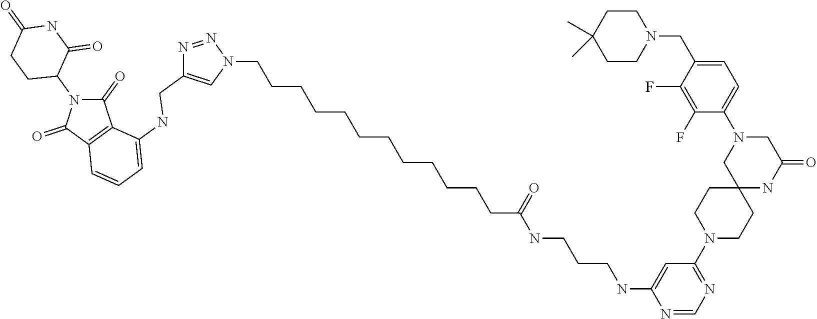 [SMILES: CC1(C)CCN(Cc2ccc(N3CC(=O)NC4(CCN(c5cc(NCCCNC(=O)CCCCCCCCCCCCn6cc(CNc7cccc8c7C(=O)N(C7CCC(=O)NC7=O)C8=O)nn6)ncn5)CC4)C3)c(F)c2F)CC1] |
| 114 | 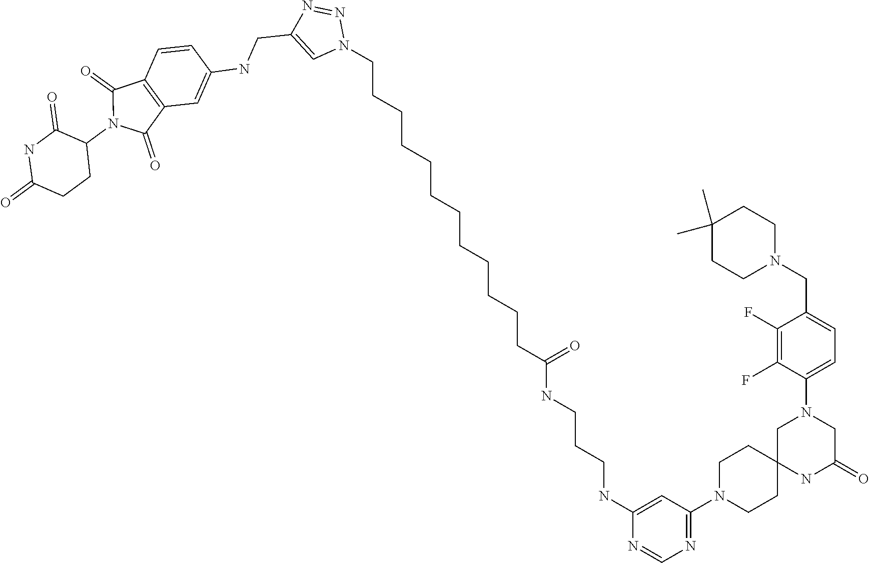 [SMILES: CC1(C)CCN(Cc2ccc(N3CC(=O)NC4(CCN(c5cc(NCCCNC(=O)CCCCCCCCCCCCn6cc(CNc7ccc8c(c7)C(=O)N(C7CCC(=O)NC7=O)C8=O)nn6)ncn5)CC4)C3)c(F)c2F)CC1] |
| 115 | 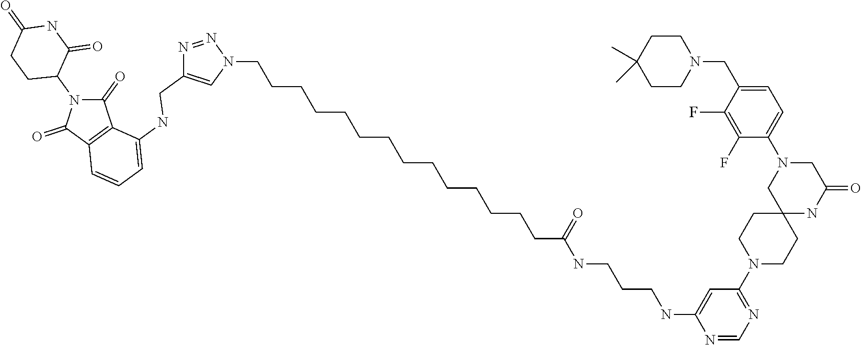 [SMILES: CC1(C)CCN(Cc2ccc(N3CC(=O)NC4(CCN(c5cc(NCCCNC(=O)CCCCCCCCCCCCCCn6cc(CNc7cccc8c7C(=O)N(C7CCC(=O)NC7=O)C8=O)nn6)ncn5)CC4)C3)c(F)c2F)CC1] |
| 116 | 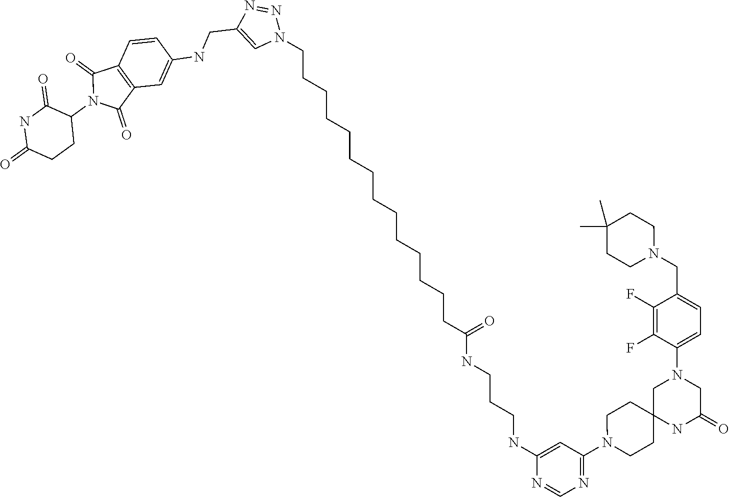 [SMILES: CC1(C)CCN(Cc2ccc(N3CC(=O)NC4(CCN(c5cc(NCCCNC(=O)CCCCCCCCCCCCCCn6cc(CNc7ccc8c(c7)C(=O)N(C7CCC(=O)NC7=O)C8=O)nn6)ncn5)CC4)C3)c(F)c2F)CC1] |
| 117 | 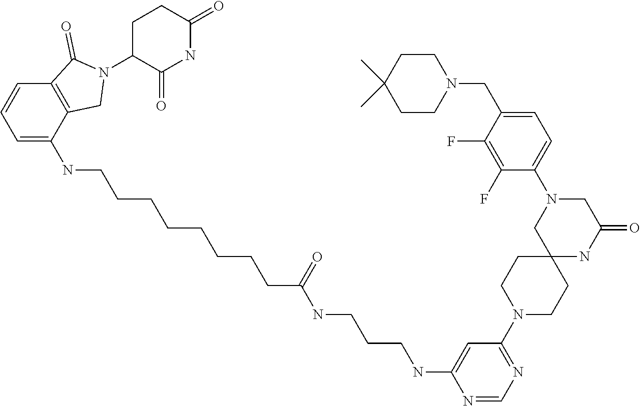 [SMILES: CC1(C)CCN(Cc2ccc(N3CC(=O)NC4(CCN(c5cc(NCCCNC(=O)CCCCCCCCNc6cccc7c6CN(C6CCC(=O)NC6=O)C7=O)ncn5)CC4)C3)c(F)c2F)CC1] |
| 118 | 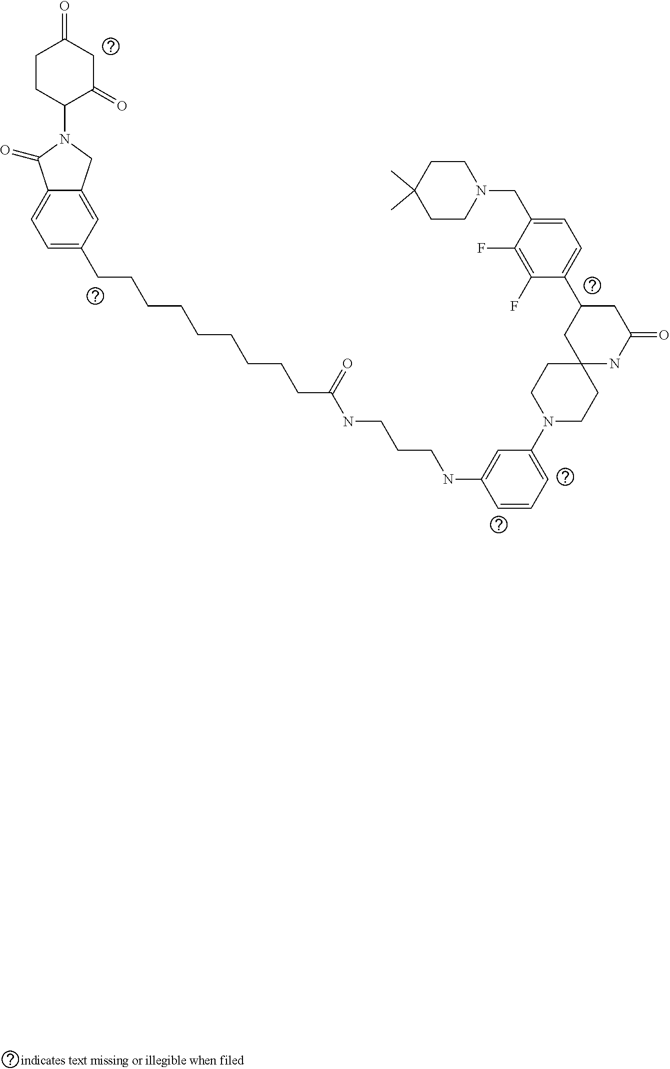 [SMILES: CC1(C)CCN(Cc2ccc(C3CC(=O)NC4(CCN(c5cccc(NCCCNC(=O)CCCCCCCCCc6ccc7c(c6)CN(C6CCC(=O)CC6=O)C7=O)c5)CC4)C3)c(F)c2F)CC1] |
| 119 | 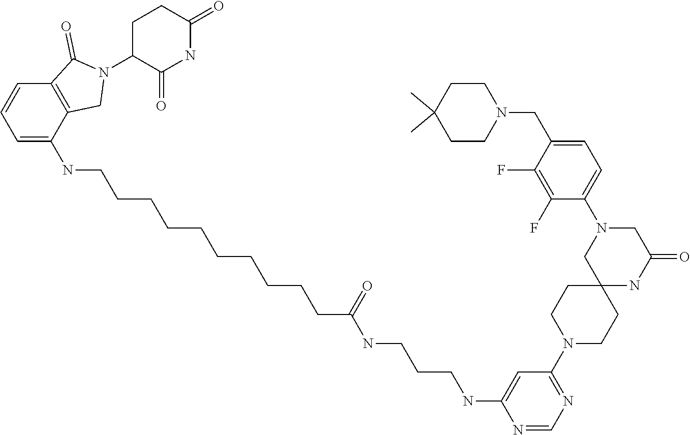 [SMILES: CC1(C)CCN(Cc2ccc(N3CC(=O)NC4(CCN(c5cc(NCCCNC(=O)CCCCCCCCCCNc6cccc7c6CN(C6CCC(=O)NC6=O)C7=O)ncn5)CC4)C3)c(F)c2F)CC1] |
| 120 | 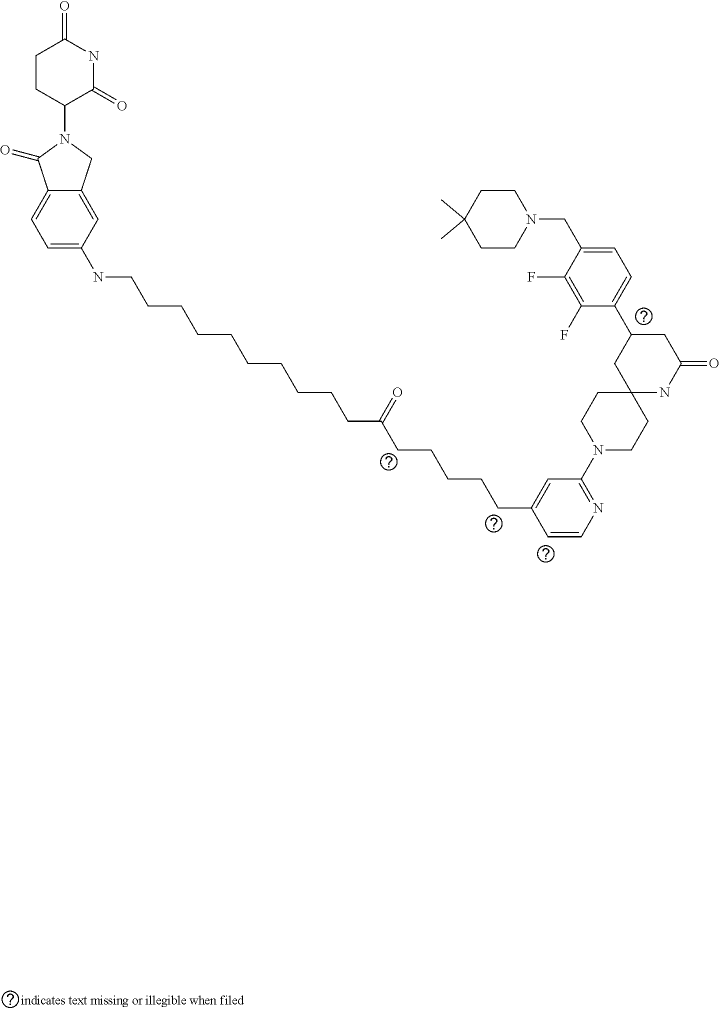 [SMILES: CC1(C)CCN(Cc2ccc(C3CC(=O)NC4(CCN(c5cc(CCCCCC(=O)CCCCCCCCCCNc6ccc7c(c6)CN(C6CCC(=O)NC6=O)C7=O)ccn5)CC4)C3)c(F)c2F)CC1] |
| 121 | 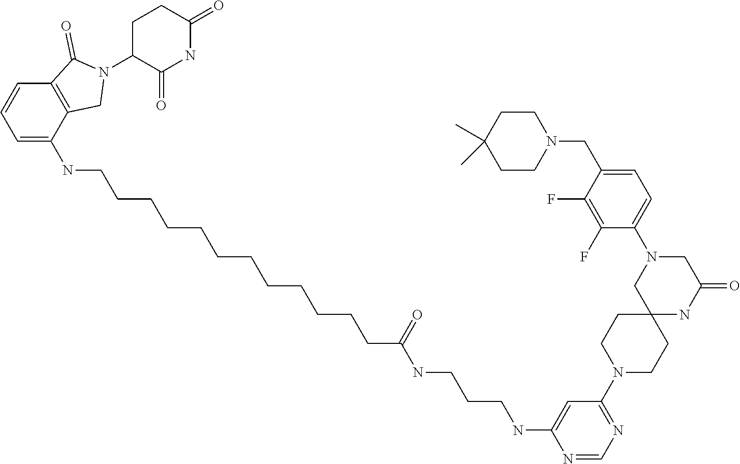 [SMILES: CC1(C)CCN(Cc2ccc(N3CC(=O)NC4(CCN(c5cc(NCCCNC(=O)CCCCCCCCCCCCNc6cccc7c6CN(C6CCC(=O)NC6=O)C7=O)ncn5)CC4)C3)c(F)c2F)CC1] |
| 122 | 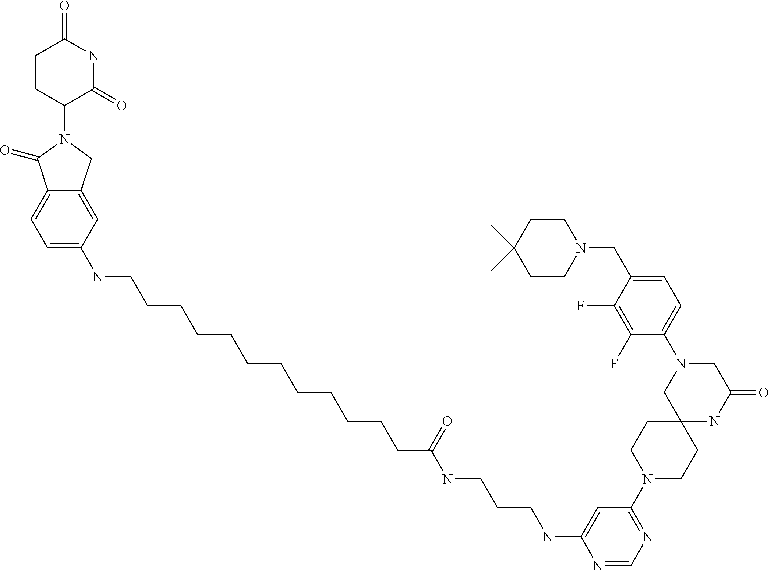 [SMILES: CC1(C)CCN(Cc2ccc(N3CC(=O)NC4(CCN(c5cc(NCCCNC(=O)CCCCCCCCCCCCNc6ccc7c(c6)CN(C6CCC(=O)NC6=O)C7=O)ncn5)CC4)C3)c(F)c2F)CC1] |
| 123 | 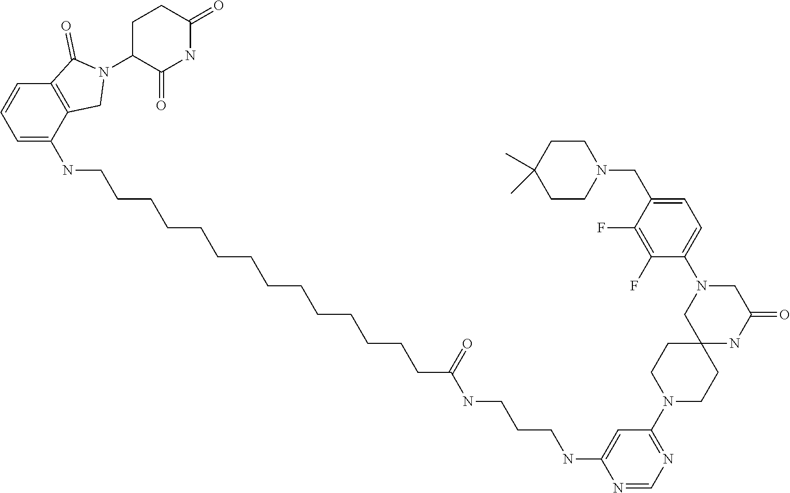 [SMILES: CC1(C)CCN(Cc2ccc(N3CC(=O)NC4(CCN(c5cc(NCCCNC(=O)CCCCCCCCCCCCCCNc6cccc7c6CN(C6CCC(=O)NC6=O)C7=O)ncn5)CC4)C3)c(F)c2F)CC1] |
| 124 | 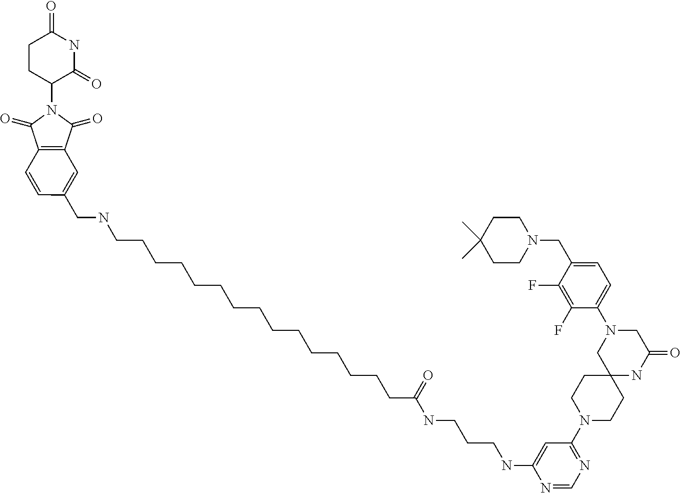 [SMILES: CC1(C)CCN(Cc2ccc(N3CC(=O)NC4(CCN(c5cc(NCCCNC(=O)CCCCCCCCCCCCCCCNCc6ccc7c(c6)C(=O)N(C6CCC(=O)NC6=O)C7=O)ncn5)CC4)C3)c(F)c2F)CC1] |
| 125 | 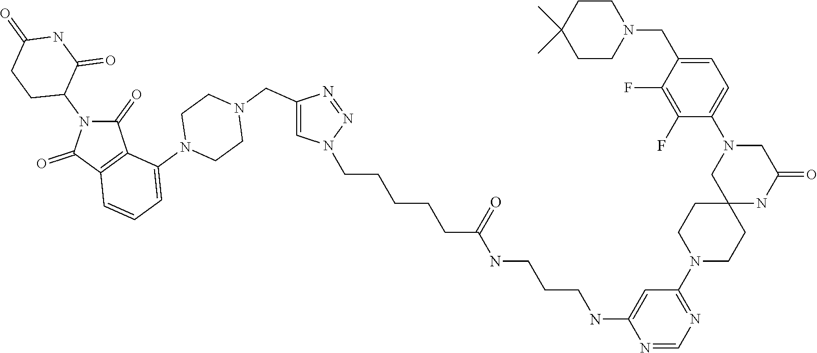 [SMILES: CC1(C)CCN(Cc2ccc(N3CC(=O)NC4(CCN(c5cc(NCCCNC(=O)CCCCCn6cc(CN7CCN(c8cccc9c8C(=O)N(C8CCC(=O)NC8=O)C9=O)CC7)nn6)ncn5)CC4)C3)c(F)c2F)CC1] |
| 126 | 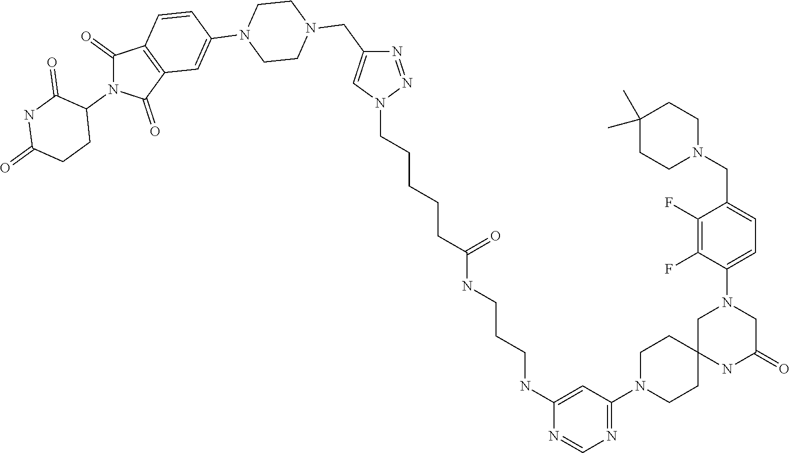 [SMILES: CC1(C)CCN(Cc2ccc(N3CC(=O)NC4(CCN(c5cc(NCCCNC(=O)CCCCCn6cc(CN7CCN(c8ccc9c(c8)C(=O)N(C8CCC(=O)NC8=O)C9=O)CC7)nn6)ncn5)CC4)C3)c(F)c2F)CC1] |
| 127 | 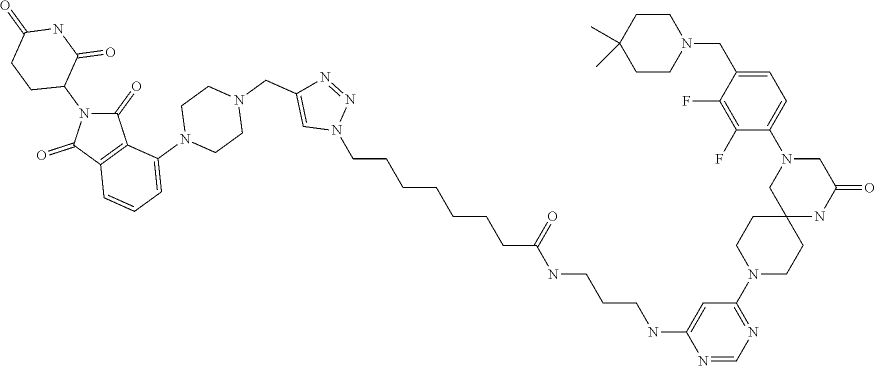 [SMILES: CC1(C)CCN(Cc2ccc(N3CC(=O)NC4(CCN(c5cc(NCCCNC(=O)CCCCCCCn6cc(CN7CCN(c8cccc9c8C(=O)N(C8CCC(=O)NC8=O)C9=O)CC7)nn6)ncn5)CC4)C3)c(F)c2F)CC1] |
| 128 | 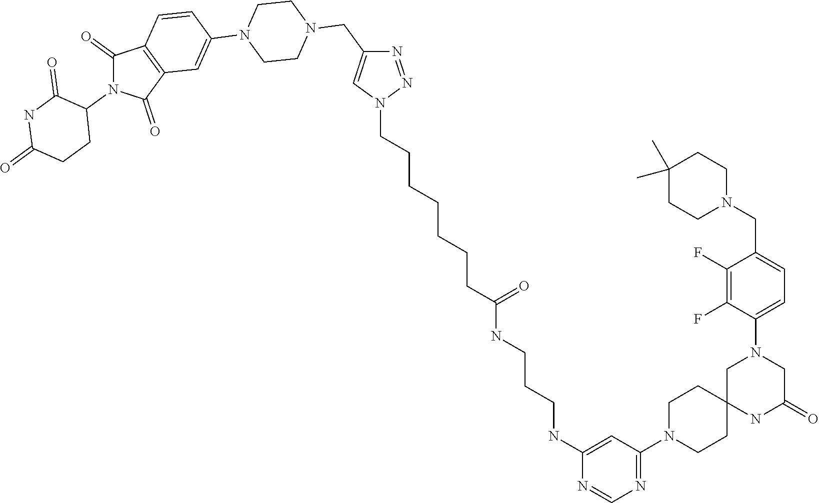 [SMILES: CC1(C)CCN(Cc2ccc(N3CC(=O)NC4(CCN(c5cc(NCCCNC(=O)CCCCCCCn6cc(CN7CCN(c8ccc9c(c8)C(=O)N(C8CCC(=O)NC8=O)C9=O)CC7)nn6)ncn5)CC4)C3)c(F)c2F)CC1] |
| 129 | 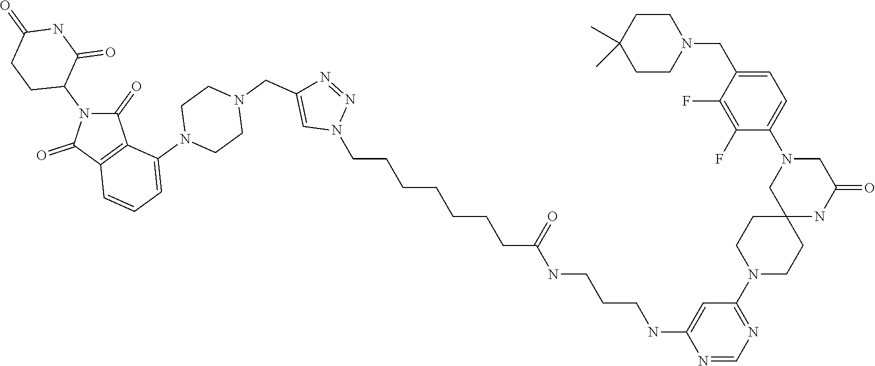 [SMILES: CC1(C)CCN(Cc2ccc(N3CC(=O)NC4(CCN(c5cc(NCCCNC(=O)CCCCCCCn6cc(CN7CCN(c8cccc9c8C(=O)N(C8CCC(=O)NC8=O)C9=O)CC7)nn6)ncn5)CC4)C3)c(F)c2F)CC1] |
| 130 | 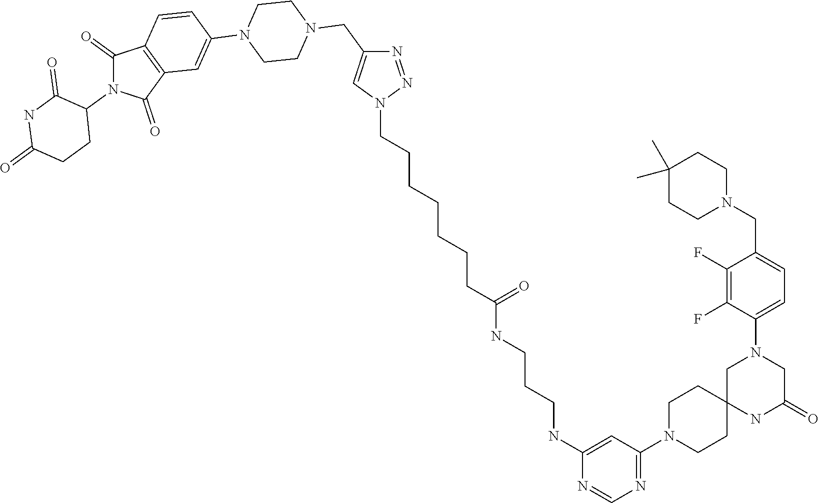 [SMILES: CC1(C)CCN(Cc2ccc(N3CC(=O)NC4(CCN(c5cc(NCCCNC(=O)CCCCCCCn6cc(CN7CCN(c8ccc9c(c8)C(=O)N(C8CCC(=O)NC8=O)C9=O)CC7)nn6)ncn5)CC4)C3)c(F)c2F)CC1] |
| 131 | 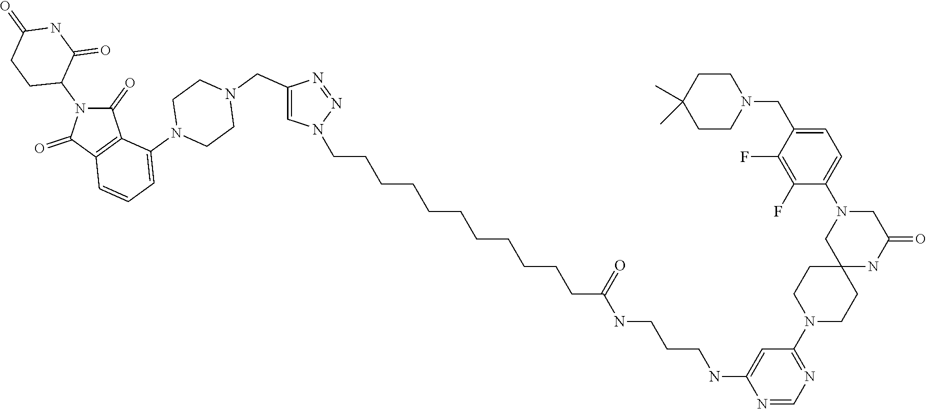 [SMILES: CC1(C)CCN(Cc2ccc(N3CC(=O)NC4(CCN(c5cc(NCCCNC(=O)CCCCCCCCCCCn6cc(CN7CCN(c8cccc9c8C(=O)N(C8CCC(=O)NC8=O)C9=O)CC7)nn6)ncn5)CC4)C3)c(F)c2F)CC1] |
| 132 | 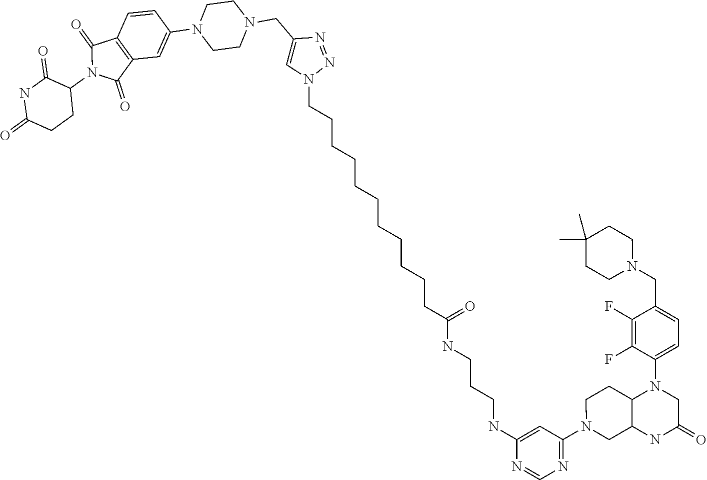 [SMILES: CC1(C)CCN(Cc2ccc(N3CC(=O)NC4CN(c5cc(NCCCNC(=O)CCCCCCCCCCCn6cc(CN7CCN(c8ccc9c(c8)C(=O)N(C8CCC(=O)NC8=O)C9=O)CC7)nn6)ncn5)CCC43)c(F)c2F)CC1] |
| 133 | 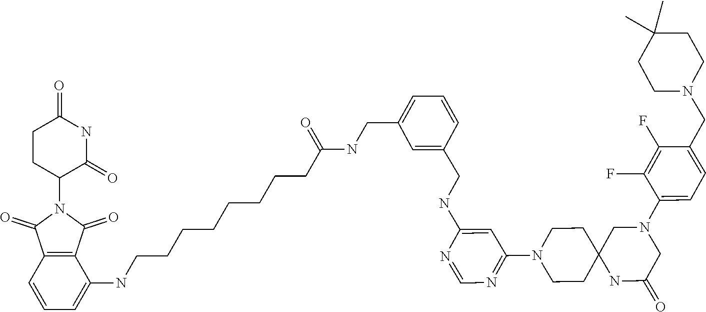 [SMILES: CC1(C)CCN(Cc2ccc(N3CC(=O)NC4(CCN(c5cc(NCc6cccc(CNC(=O)CCCCCCCCNc7cccc8c7C(=O)N(C7CCC(=O)NC7=O)C8=O)c6)ncn5)CC4)C3)c(F)c2F)CC1] |
| 134 | 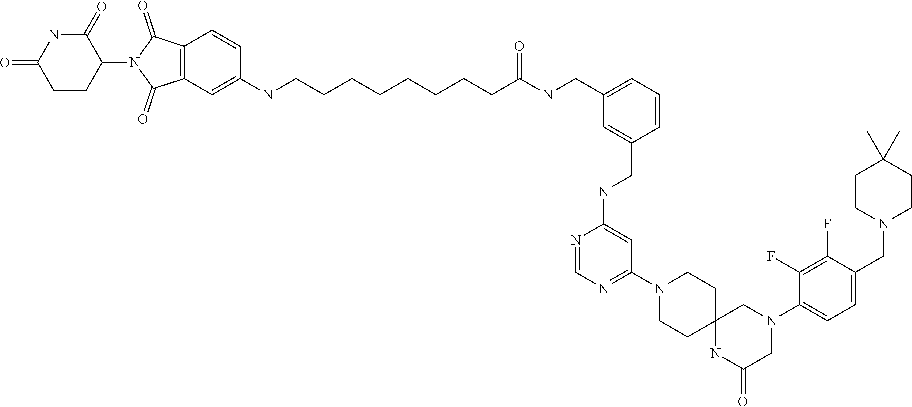 [SMILES: CC1(C)CCN(Cc2ccc(N3CC(=O)NC4(CCN(c5cc(NCc6cccc(CNC(=O)CCCCCCCCNc7ccc8c(c7)C(=O)N(C7CCC(=O)NC7=O)C8=O)c6)ncn5)CC4)C3)c(F)c2F)CC1] |
| 135 | 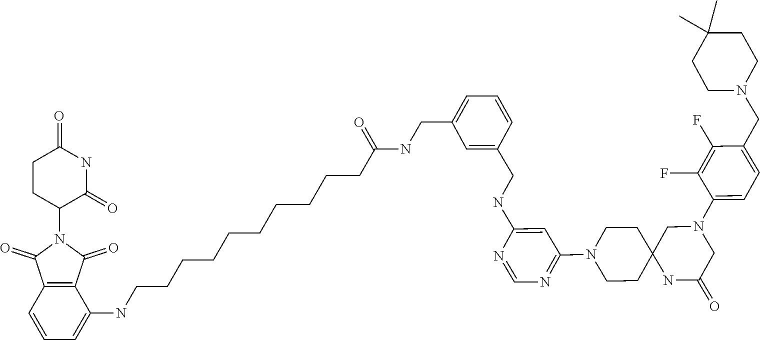 [SMILES: CC1(C)CCN(Cc2ccc(N3CC(=O)NC4(CCN(c5cc(NCc6cccc(CNC(=O)CCCCCCCCCCNc7cccc8c7C(=O)N(C7CCC(=O)NC7=O)C8=O)c6)ncn5)CC4)C3)c(F)c2F)CC1] |
| 136 | 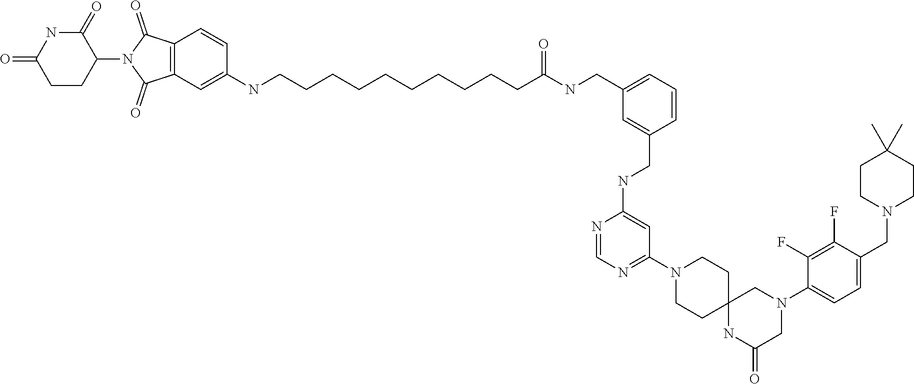 [SMILES: CC1(C)CCN(Cc2ccc(N3CC(=O)NC4(CCN(c5cc(NCc6cccc(CNC(=O)CCCCCCCCCCNc7ccc8c(c7)C(=O)N(C7CCC(=O)NC7=O)C8=O)c6)ncn5)CC4)C3)c(F)c2F)CC1] |
| 137 | 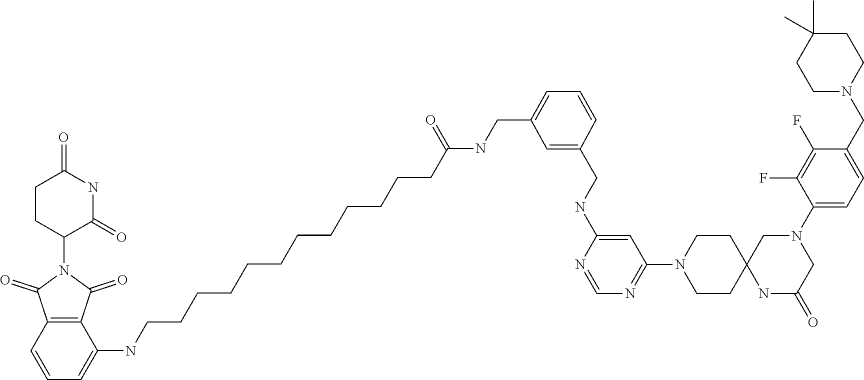 [SMILES: CC1(C)CCN(Cc2ccc(N3CC(=O)NC4(CCN(c5cc(NCc6cccc(CNC(=O)CCCCCCCCCCCCNc7cccc8c7C(=O)N(C7CCC(=O)NC7=O)C8=O)c6)ncn5)CC4)C3)c(F)c2F)CC1] |
| 138 | 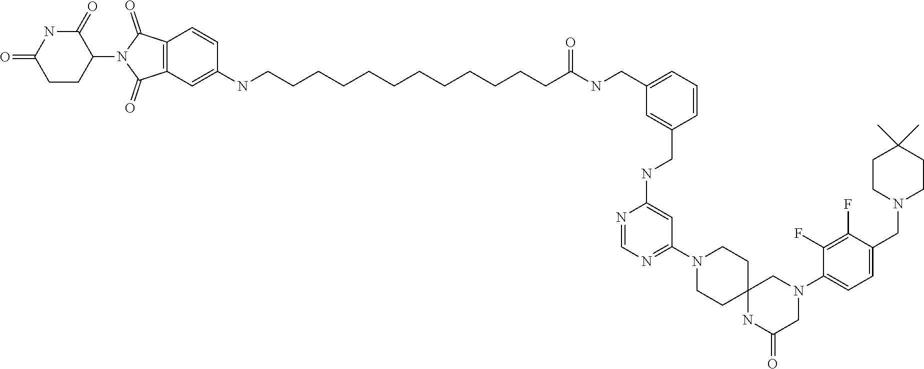 [SMILES: CC1(C)CCN(Cc2ccc(N3CC(=O)NC4(CCN(c5cc(NCc6cccc(CNC(=O)CCCCCCCCCCCCNc7ccc8c(c7)C(=O)N(C7CCC(=O)NC7=O)C8=O)c6)ncn5)CC4)C3)c(F)c2F)CC1] |
| 139 | 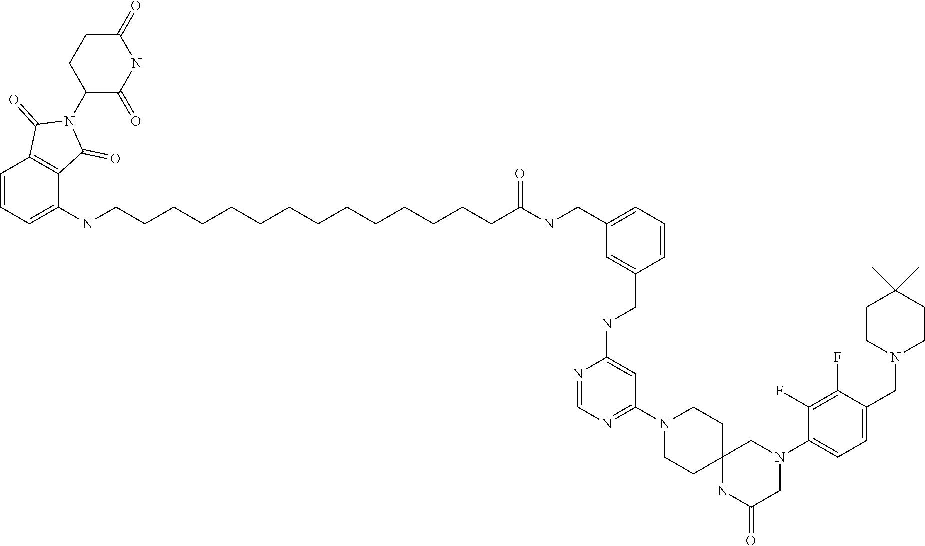 [SMILES: CC1(C)CCN(Cc2ccc(N3CC(=O)NC4(CCN(c5cc(NCc6cccc(CNC(=O)CCCCCCCCCCCCCCNc7cccc8c7C(=O)N(C7CCC(=O)NC7=O)C8=O)c6)ncn5)CC4)C3)c(F)c2F)CC1] |
| 140 | 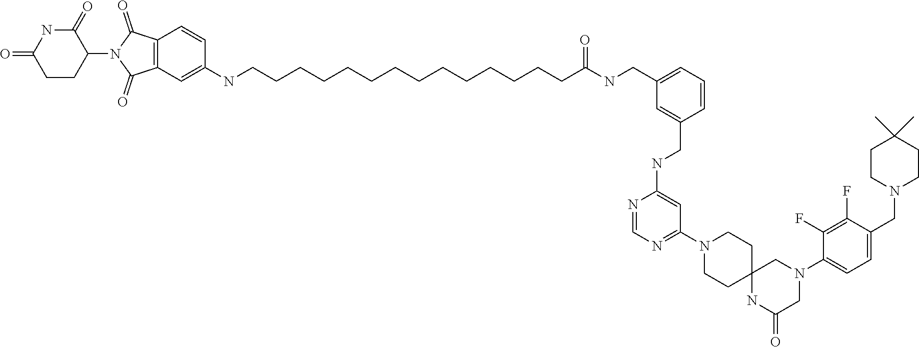 [SMILES: CC1(C)CCN(Cc2ccc(N3CC(=O)NC4(CCN(c5cc(NCc6cccc(CNC(=O)CCCCCCCCCCCCCCNc7ccc8c(c7)C(=O)N(C7CCC(=O)NC7=O)C8=O)c6)ncn5)CC4)C3)c(F)c2F)CC1] |
| 141 | 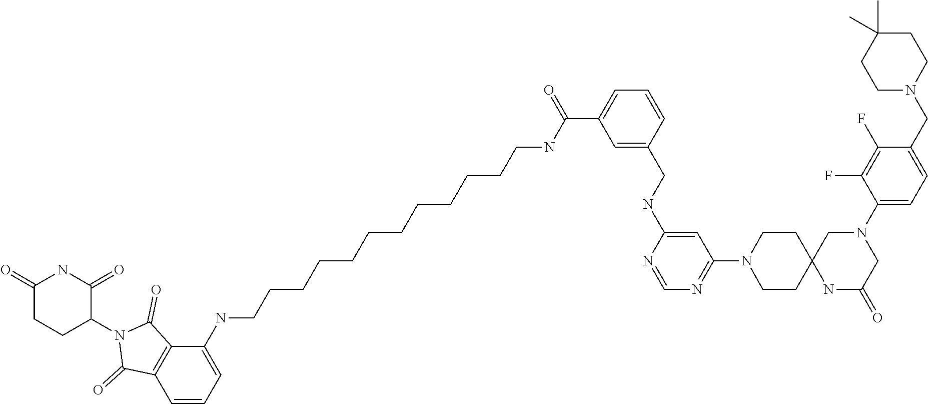 [SMILES: CC1(C)CCN(Cc2ccc(N3CC(=O)NC4(CCN(c5cc(NCc6cccc(C(=O)NCCCCCCCCCCCCNc7cccc8c7C(=O)N(C7CCC(=O)NC7=O)C8=O)c6)ncn5)CC4)C3)c(F)c2F)CC1] |
| 142 | 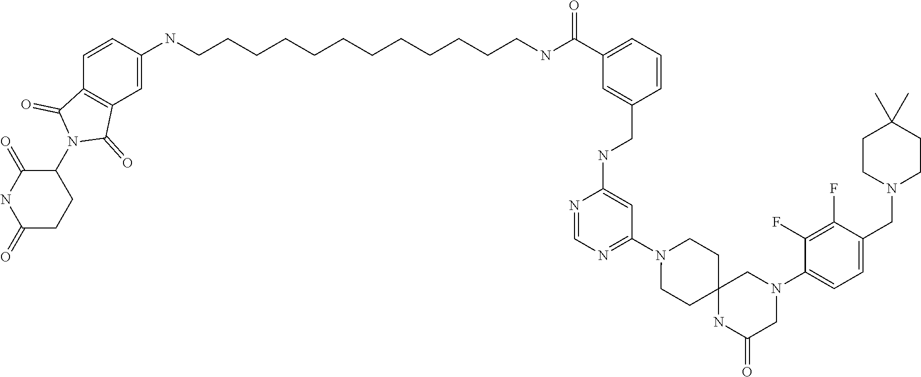 [SMILES: CC1(C)CCN(Cc2ccc(N3CC(=O)NC4(CCN(c5cc(NCc6cccc(C(=O)NCCCCCCCCCCCCNc7ccc8c(c7)C(=O)N(C7CCC(=O)NC7=O)C8=O)c6)ncn5)CC4)C3)c(F)c2F)CC1] |
| 143 | 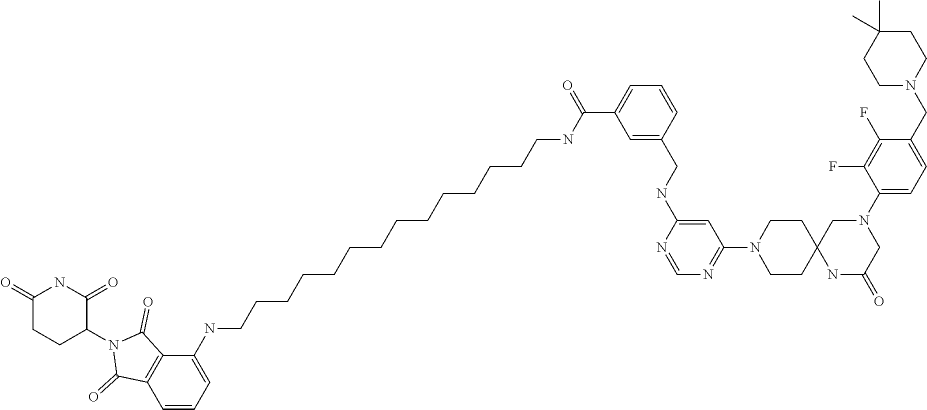 [SMILES: CC1(C)CCN(Cc2ccc(N3CC(=O)NC4(CCN(c5cc(NCc6cccc(C(=O)NCCCCCCCCCCCCCCNc7cccc8c7C(=O)N(C7CCC(=O)NC7=O)C8=O)c6)ncn5)CC4)C3)c(F)c2F)CC1] |
| 144 | 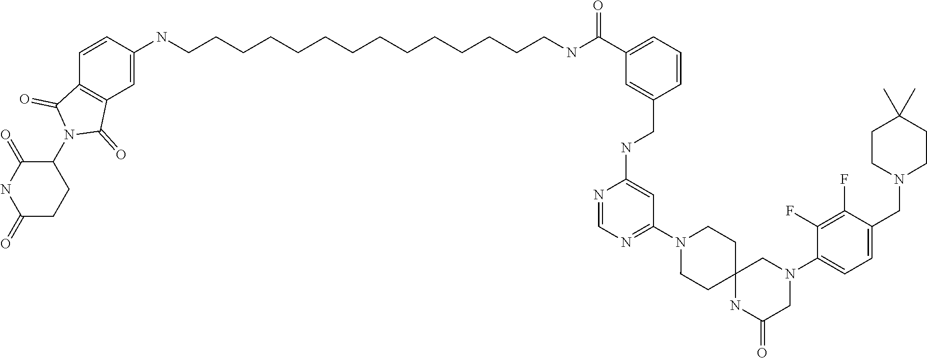 [SMILES: CC1(C)CCN(Cc2ccc(N3CC(=O)NC4(CCN(c5cc(NCc6cccc(C(=O)NCCCCCCCCCCCCCCNc7ccc8c(c7)C(=O)N(C7CCC(=O)NC7=O)C8=O)c6)ncn5)CC4)C3)c(F)c2F)CC1] |
| 145 | 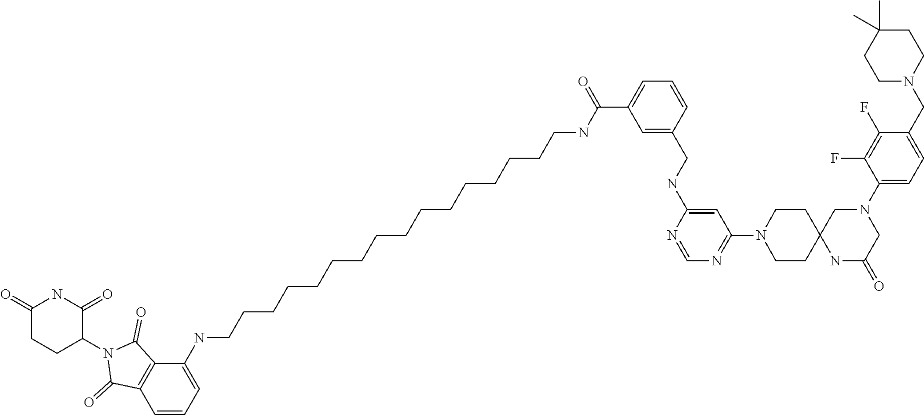 [SMILES: CC1(C)CCN(Cc2ccc(N3CC(=O)NC4(CCN(c5cc(NCc6cccc(C(=O)NCCCCCCCCCCCCCCCCNc7cccc8c7C(=O)N(C7CCC(=O)NC7=O)C8=O)c6)ncn5)CC4)C3)c(F)c2F)CC1] |
| 146 | 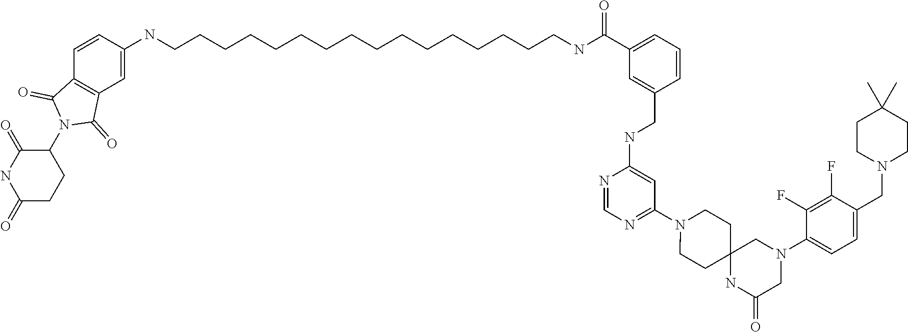 [SMILES: CC1(C)CCN(Cc2ccc(N3CC(=O)NC4(CCN(c5cc(NCc6cccc(C(=O)NCCCCCCCCCCCCCCCCNc7ccc8c(c7)C(=O)N(C7CCC(=O)NC7=O)C8=O)c6)ncn5)CC4)C3)c(F)c2F)CC1] |
| 147 | 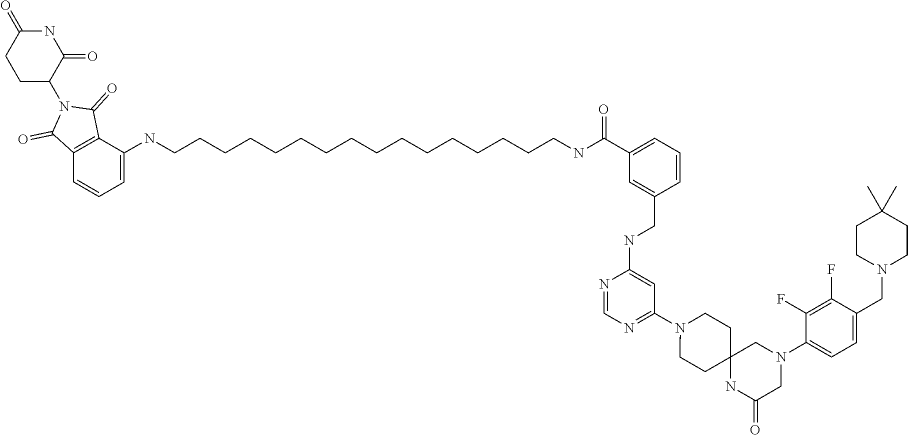 [SMILES: CC1(C)CCN(Cc2ccc(N3CC(=O)NC4(CCN(c5cc(NCc6cccc(C(=O)NCCCCCCCCCCCCCCCCNc7cccc8c7C(=O)N(C7CCC(=O)NC7=O)C8=O)c6)ncn5)CC4)C3)c(F)c2F)CC1] |
| 148 | 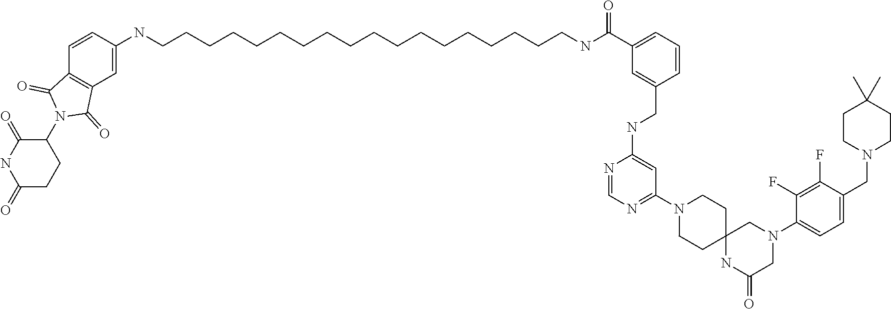 [SMILES: CC1(C)CCN(Cc2ccc(N3CC(=O)NC4(CCN(c5cc(NCc6cccc(C(=O)NCCCCCCCCCCCCCCCCCCNc7ccc8c(c7)C(=O)N(C7CCC(=O)NC7=O)C8=O)c6)ncn5)CC4)C3)c(F)c2F)CC1] |
| 149 | 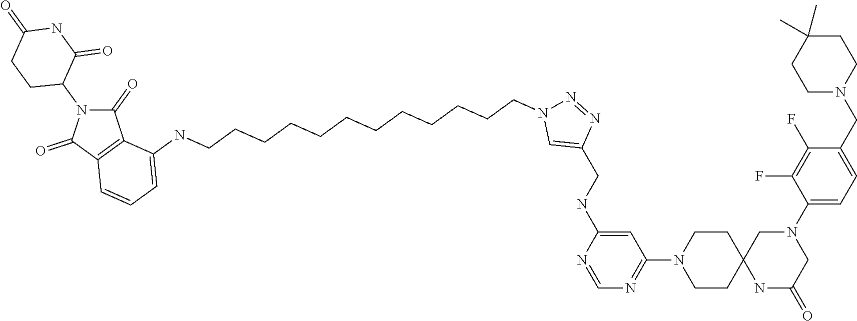 [SMILES: CC1(C)CCN(Cc2ccc(N3CC(=O)NC4(CCN(c5cc(NCc6cn(CCCCCCCCCCCCNc7cccc8c7C(=O)N(C7CCC(=O)NC7=O)C8=O)nn6)ncn5)CC4)C3)c(F)c2F)CC1] |
| 150 | 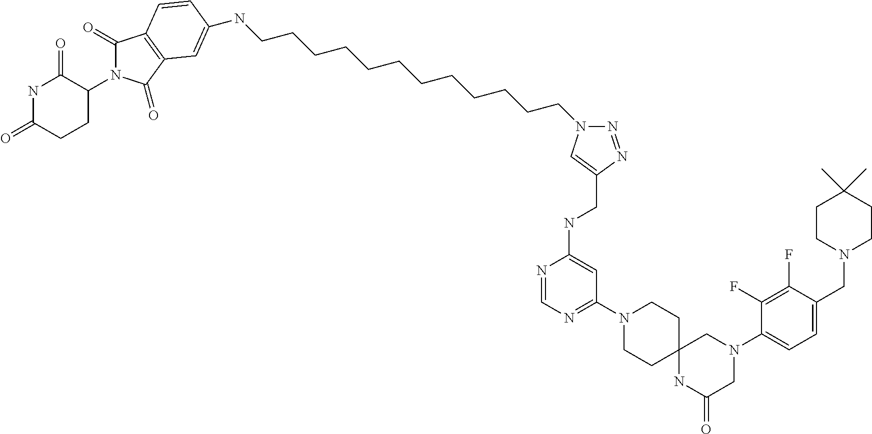 [SMILES: CC1(C)CCN(Cc2ccc(N3CC(=O)NC4(CCN(c5cc(NCc6cn(CCCCCCCCCCCCNc7ccc8c(c7)C(=O)N(C7CCC(=O)NC7=O)C8=O)nn6)ncn5)CC4)C3)c(F)c2F)CC1] |
| 151 | 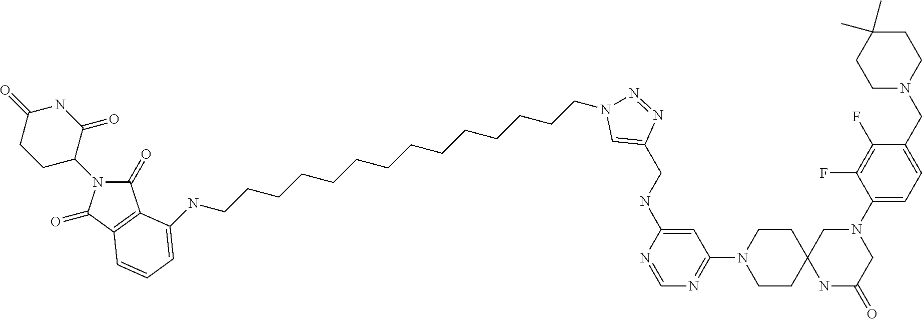 [SMILES: CC1(C)CCN(Cc2ccc(N3CC(=O)NC4(CCN(c5cc(NCc6cn(CCCCCCCCCCCCCCNc7cccc8c7C(=O)N(C7CCC(=O)NC7=O)C8=O)nn6)ncn5)CC4)C3)c(F)c2F)CC1] |
| 152 | 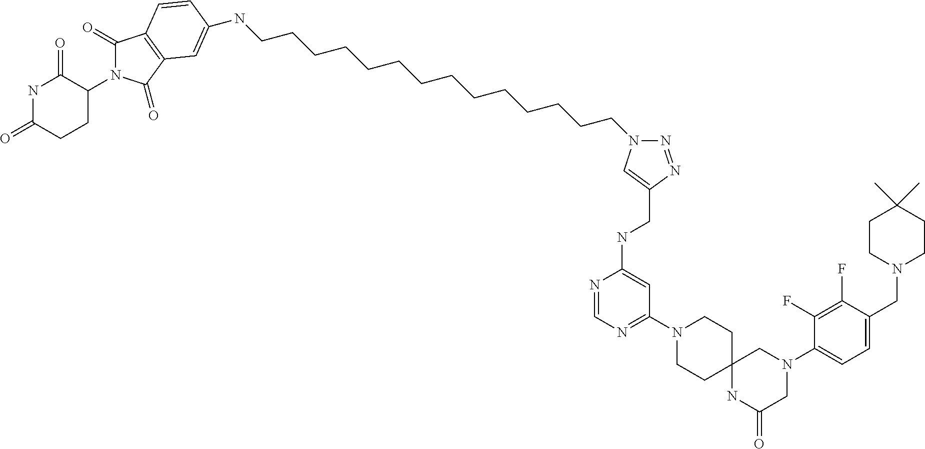 [SMILES: CC1(C)CCN(Cc2ccc(N3CC(=O)NC4(CCN(c5cc(NCc6cn(CCCCCCCCCCCCCCNc7ccc8c(c7)C(=O)N(C7CCC(=O)NC7=O)C8=O)nn6)ncn5)CC4)C3)c(F)c2F)CC1] |
| 153 | 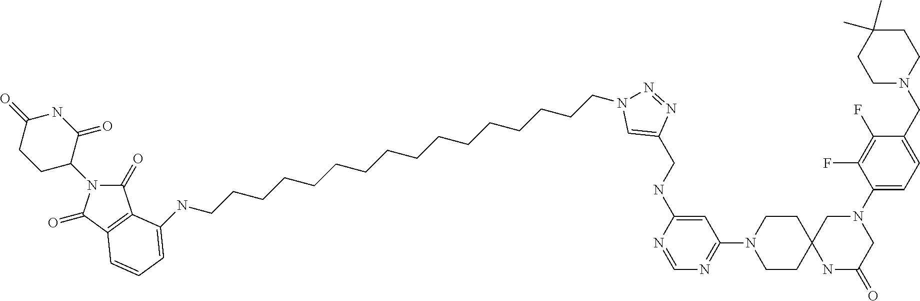 [SMILES: CC1(C)CCN(Cc2ccc(N3CC(=O)NC4(CCN(c5cc(NCc6cn(CCCCCCCCCCCCCCCCNc7cccc8c7C(=O)N(C7CCC(=O)NC7=O)C8=O)nn6)ncn5)CC4)C3)c(F)c2F)CC1] |
| 154 | 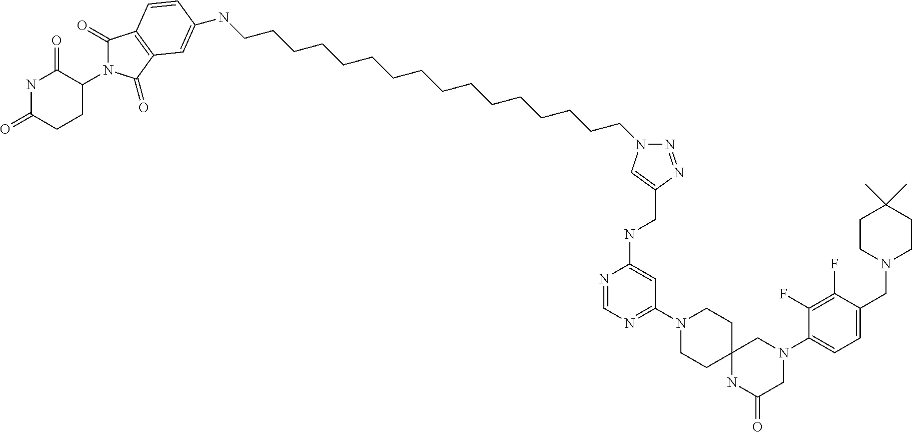 [SMILES: CC1(C)CCN(Cc2ccc(N3CC(=O)NC4(CCN(c5cc(NCc6cn(CCCCCCCCCCCCCCCCNc7ccc8c(c7)C(=O)N(C7CCC(=O)NC7=O)C8=O)nn6)ncn5)CC4)C3)c(F)c2F)CC1] |
| 155 | 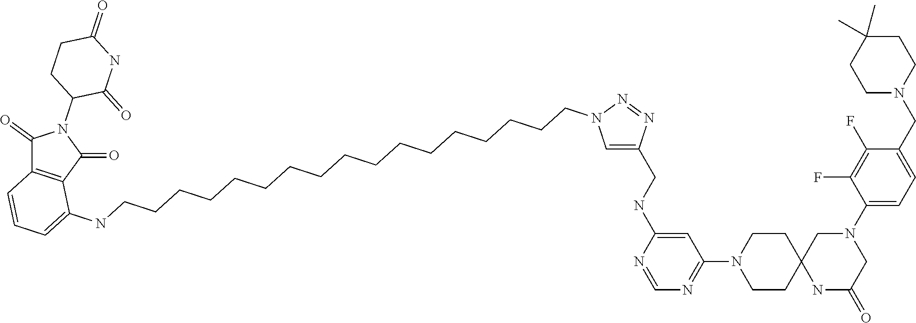 [SMILES: CC1(C)CCN(Cc2ccc(N3CC(=O)NC4(CCN(c5cc(NCc6cn(CCCCCCCCCCCCCCCCCNc7cccc8c7C(=O)N(C7CCC(=O)NC7=O)C8=O)nn6)ncn5)CC4)C3)c(F)c2F)CC1] |
| 156 | 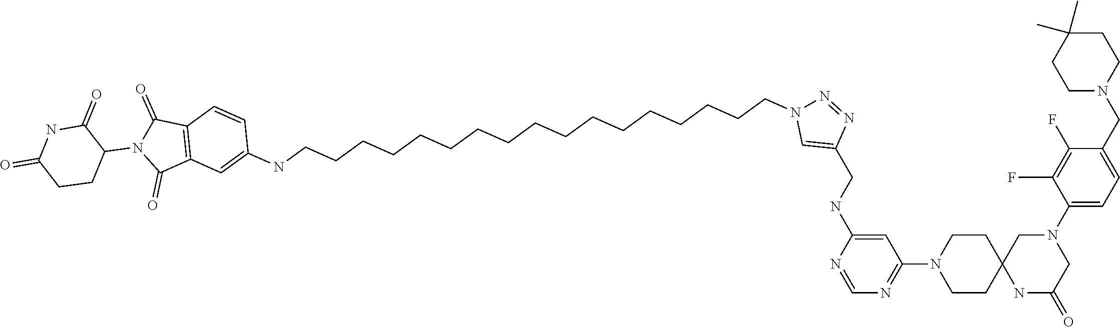 [SMILES: CC1(C)CCN(Cc2ccc(N3CC(=O)NC4(CCN(c5cc(NCc6cn(CCCCCCCCCCCCCCCCCNc7ccc8c(c7)C(=O)N(C7CCC(=O)NC7=O)C8=O)nn6)ncn5)CC4)C3)c(F)c2F)CC1] |
| 157 | 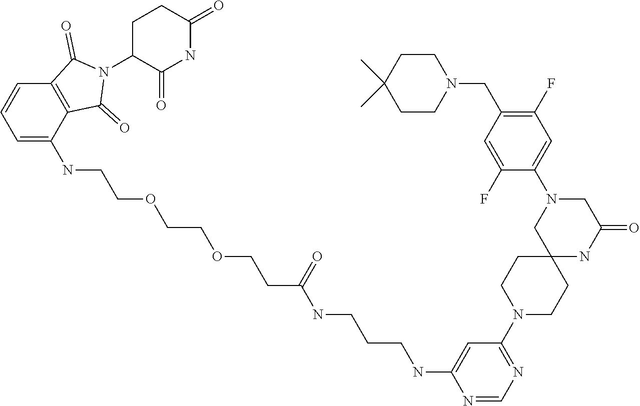 [SMILES: CC1(C)CCN(Cc2cc(F)c(N3CC(=O)NC4(CCN(c5cc(NCCCNC(=O)CCOCCOCCNc6cccc7c6C(=O)N(C6CCC(=O)NC6=O)C7=O)ncn5)CC4)C3)cc2F)CC1] |
| 158 | 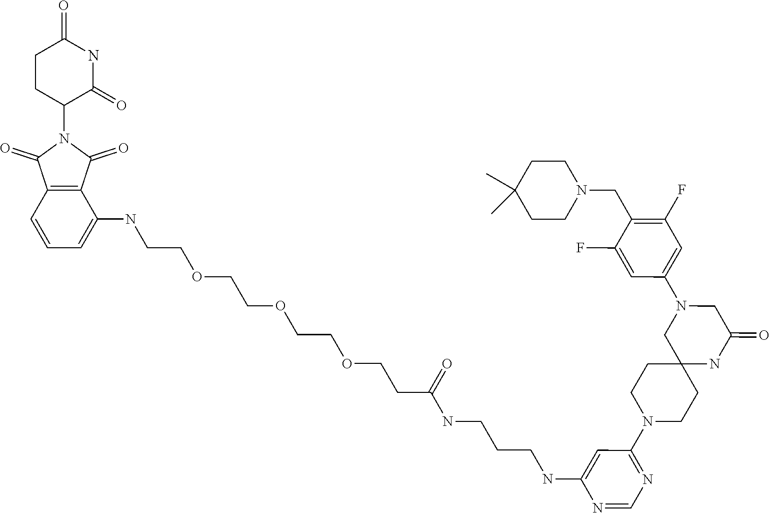 [SMILES: CC1(C)CCN(Cc2c(F)cc(N3CC(=O)NC4(CCN(c5cc(NCCCNC(=O)CCOCCOCCOCCNc6cccc7c6C(=O)N(C6CCC(=O)NC6=O)C7=O)ncn5)CC4)C3)cc2F)CC1] |
| 159 | 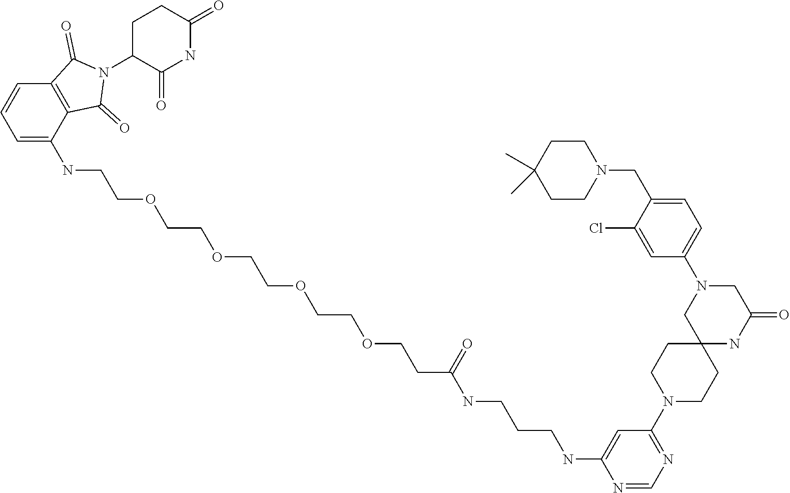 [SMILES: CC1(C)CCN(Cc2ccc(N3CC(=O)NC4(CCN(c5cc(NCCCNC(=O)CCOCCOCCOCCOCCNc6cccc7c6C(=O)N(C6CCC(=O)NC6=O)C7=O)ncn5)CC4)C3)cc2Cl)CC1] |
| 160 | 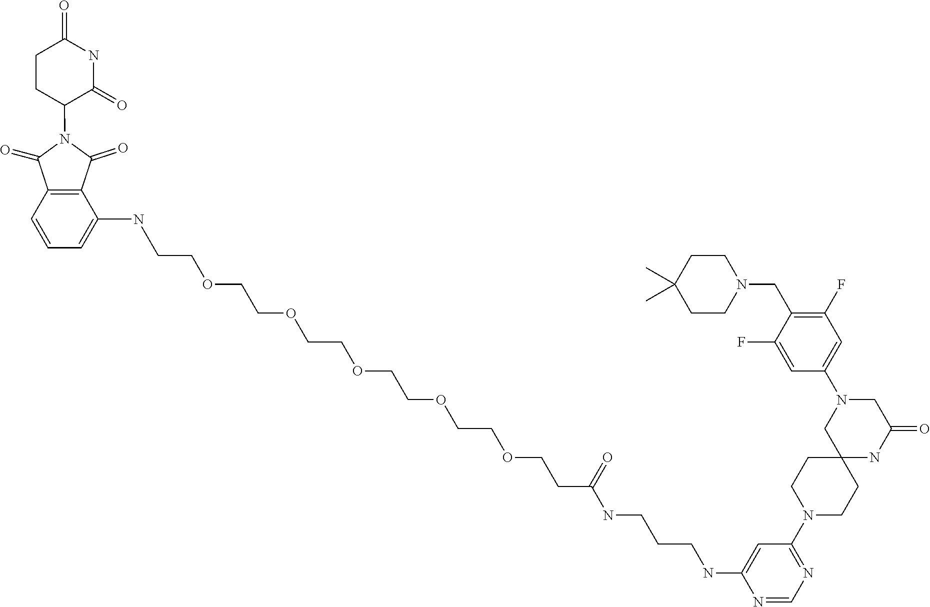 |
| 161 | 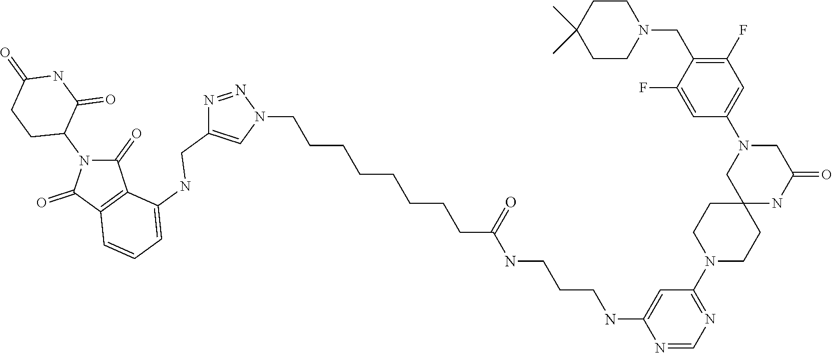 [SMILES: CC1(C)CCN(Cc2c(F)cc(N3CC(=O)NC4(CCN(c5cc(NCCCNC(=O)CCCCCCCCn6cc(CNc7cccc8c7C(=O)N(C7CCC(=O)NC7=O)C8=O)nn6)ncn5)CC4)C3)cc2F)CC1] |
| 162 | 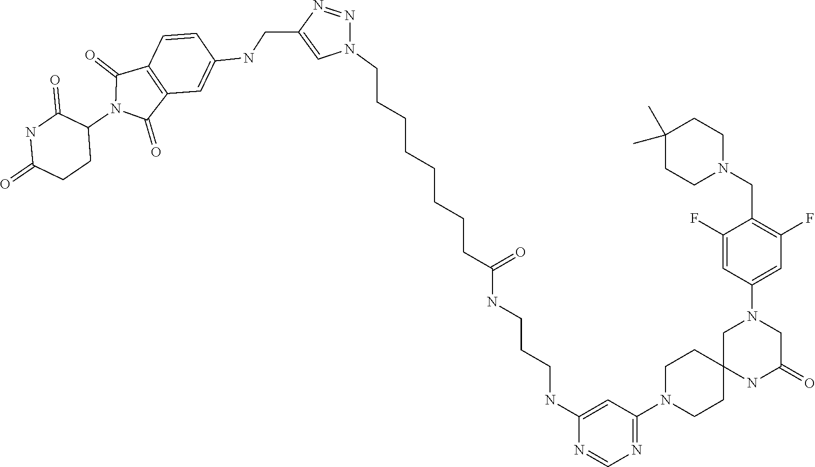 [SMILES: CC1(C)CCN(Cc2c(F)cc(N3CC(=O)NC4(CCN(c5cc(NCCCNC(=O)CCCCCCCCn6cc(CNc7ccc8c(c7)C(=O)N(C7CCC(=O)NC7=O)C8=O)nn6)ncn5)CC4)C3)cc2F)CC1] |
| 163 | 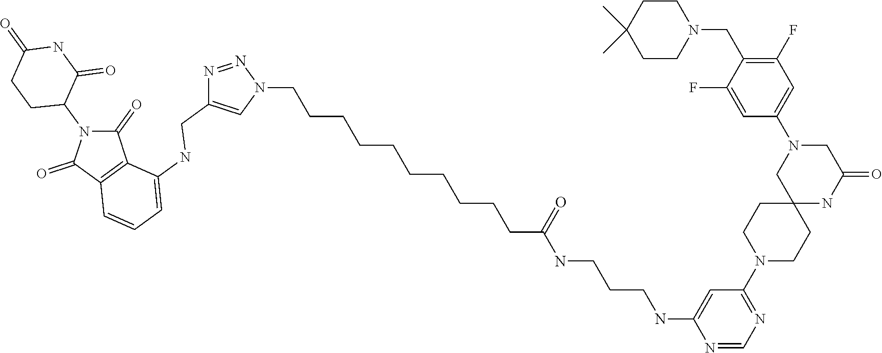 [SMILES: CC1(C)CCN(Cc2c(F)cc(N3CC(=O)NC4(CCN(c5cc(NCCCNC(=O)CCCCCCCCCCn6cc(CNc7cccc8c7C(=O)N(C7CCC(=O)NC7=O)C8=O)nn6)ncn5)CC4)C3)cc2F)CC1] |
| 164 | 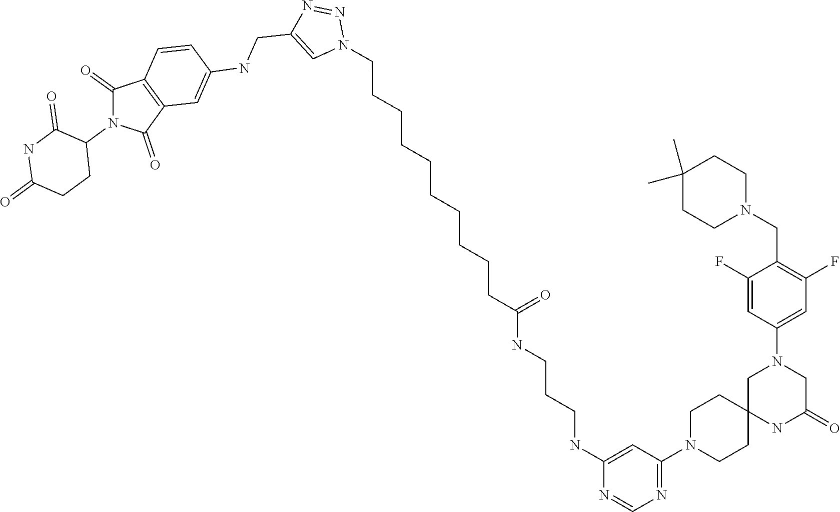 [SMILES: CC1(C)CCN(Cc2c(F)cc(N3CC(=O)NC4(CCN(c5cc(NCCCNC(=O)CCCCCCCCCCn6cc(CNc7ccc8c(c7)C(=O)N(C7CCC(=O)NC7=O)C8=O)nn6)ncn5)CC4)C3)cc2F)CC1] |
| 165 | 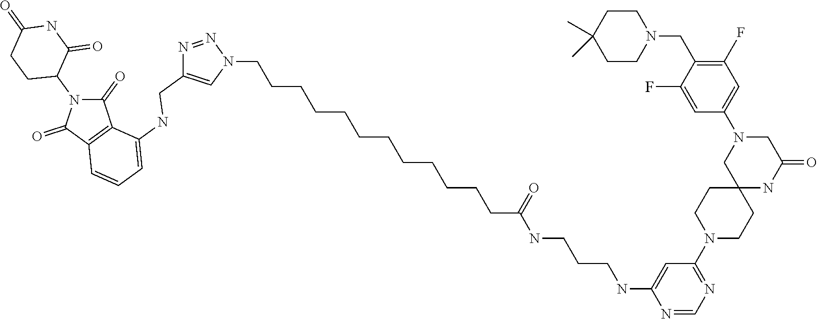 [SMILES: CC1(C)CCN(Cc2c(F)cc(N3CC(=O)NC4(CCN(c5cc(NCCCNC(=O)CCCCCCCCCCCCn6cc(CNc7cccc8c7C(=O)N(C7CCC(=O)NC7=O)C8=O)nn6)ncn5)CC4)C3)cc2F)CC1] |
| 166 | 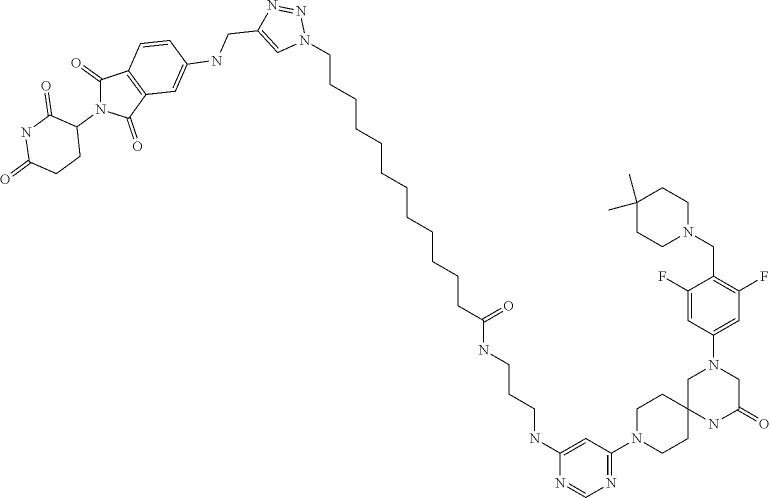 [SMILES: CC1(C)CCN(Cc2c(F)cc(N3CC(=O)NC4(CCN(c5cc(NCCCNC(=O)CCCCCCCCCCCCn6cc(CNc7ccc8c(c7)C(=O)N(C7CCC(=O)NC7=O)C8=O)nn6)ncn5)CC4)C3)cc2F)CC1] |
| 167 | 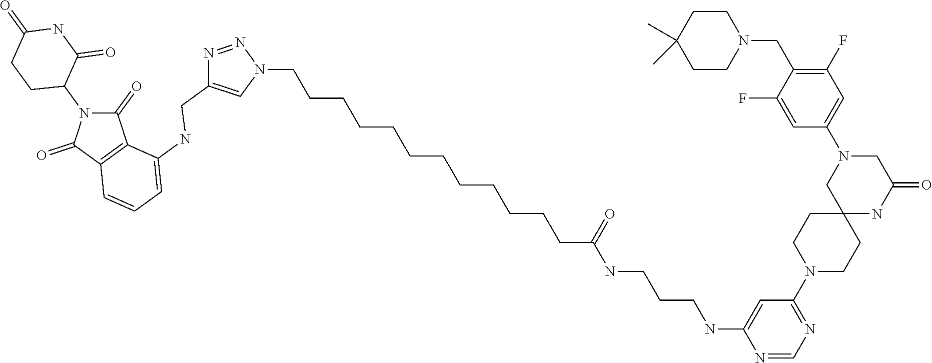 [SMILES: CC1(C)CCN(Cc2c(F)cc(N3CC(=O)NC4(CCN(c5cc(NCCCNC(=O)CCCCCCCCCCCCn6cc(CNc7cccc8c7C(=O)N(C7CCC(=O)NC7=O)C8=O)nn6)ncn5)CC4)C3)cc2F)CC1] |
| 168 | 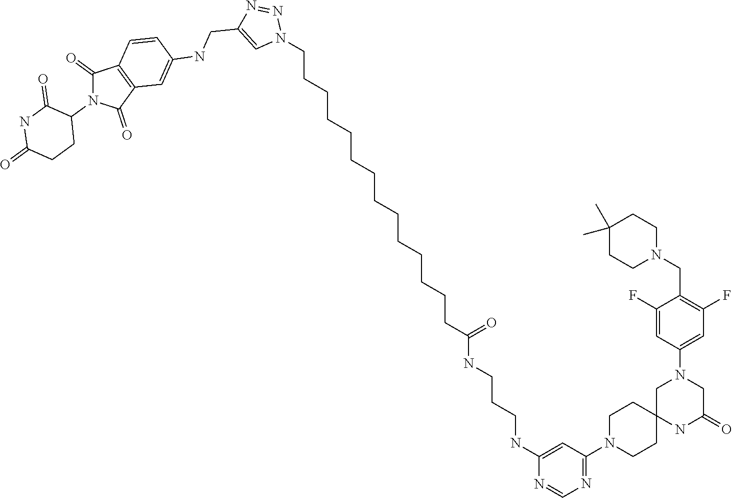 [SMILES: CC1(C)CCN(Cc2c(F)cc(N3CC(=O)NC4(CCN(c5cc(NCCCNC(=O)CCCCCCCCCCCCCCn6cc(CNc7ccc8c(c7)C(=O)N(C7CCC(=O)NC7=O)C8=O)nn6)ncn5)CC4)C3)cc2F)CC1] |
| 169 | 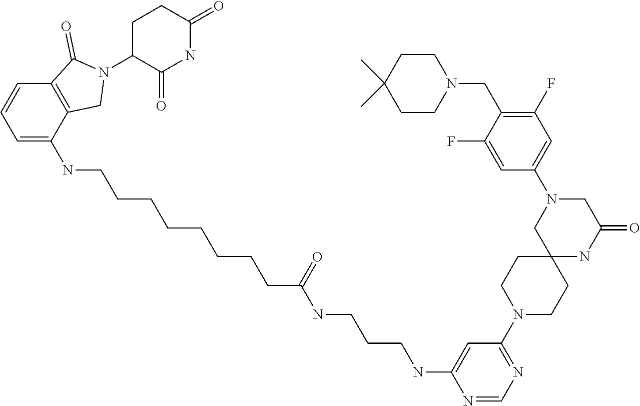 [SMILES: CC1(C)CCN(Cc2c(F)cc(N3CC(=O)NC4(CCN(c5cc(NCCCNC(=O)CCCCCCCCNc6cccc7c6CN(C6CCC(=O)NC6=O)C7=O)ncn5)CC4)C3)cc2F)CC1] |
| 170 | 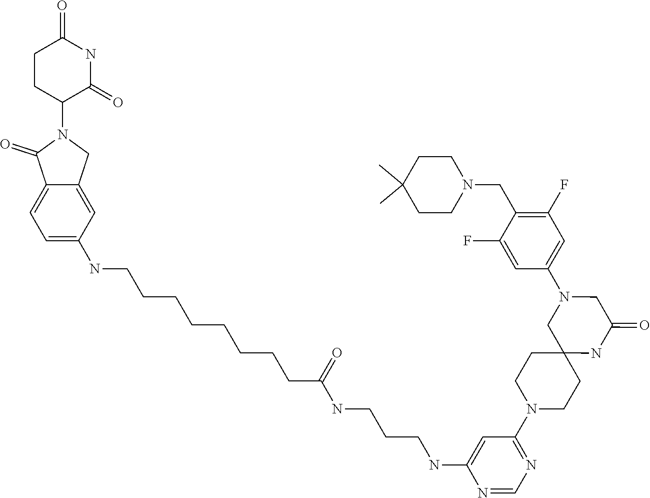 [SMILES: CC1(C)CCN(Cc2c(F)cc(N3CC(=O)NC4(CCN(c5cc(NCCCNC(=O)CCCCCCCCNc6ccc7c(c6)CN(C6CCC(=O)NC6=O)C7=O)ncn5)CC4)C3)cc2F)CC1] |
| 171 | 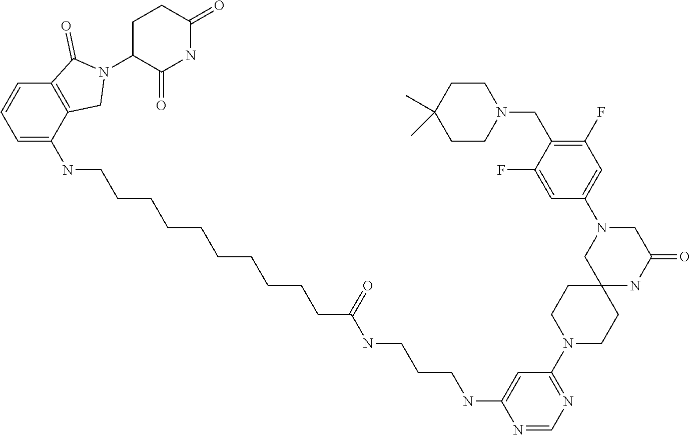 [SMILES: CC1(C)CCN(Cc2c(F)cc(N3CC(=O)NC4(CCN(c5cc(NCCCNC(=O)CCCCCCCCCCNc6cccc7c6CN(C6CCC(=O)NC6=O)C7=O)ncn5)CC4)C3)cc2F)CC1] |
| 172 | 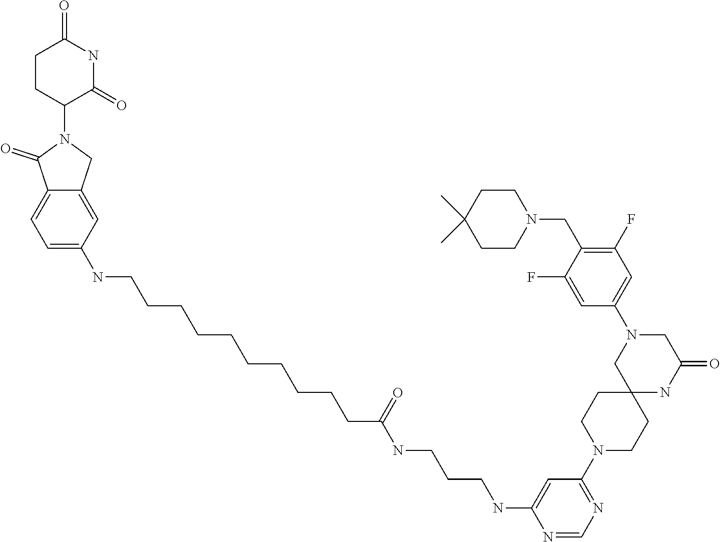 [SMILES: CC1(C)CCN(Cc2c(F)cc(N3CC(=O)NC4(CCN(c5cc(NCCCNC(=O)CCCCCCCCCCNc6ccc7c(c6)CN(C6CCC(=O)NC6=O)C7=O)ncn5)CC4)C3)cc2F)CC1] |
| 173 | 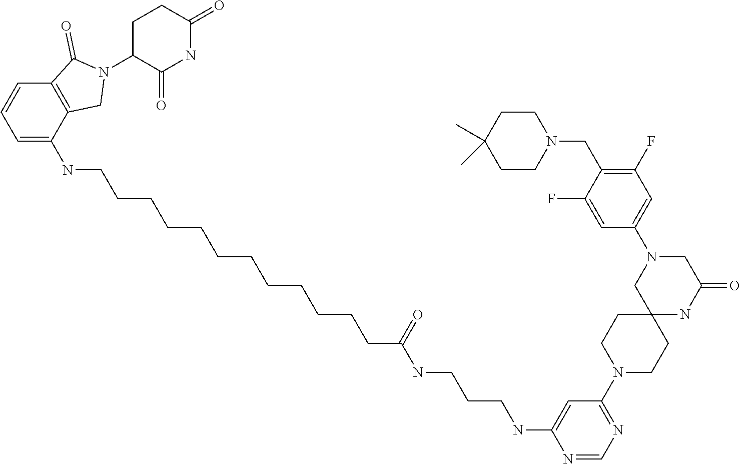 [SMILES: CC1(C)CCN(Cc2c(F)cc(N3CC(=O)NC4(CCN(c5cc(NCCCNC(=O)CCCCCCCCCCCCNc6cccc7c6CN(C6CCC(=O)NC6=O)C7=O)ncn5)CC4)C3)cc2F)CC1] |
| 174 | 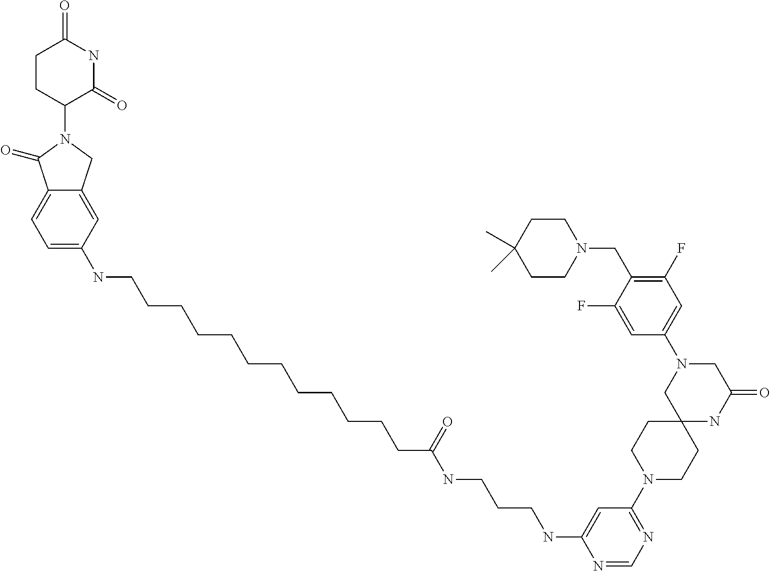 [SMILES: CC1(C)CCN(Cc2c(F)cc(N3CC(=O)NC4(CCN(c5cc(NCCCNC(=O)CCCCCCCCCCCCNc6ccc7c(c6)CN(C6CCC(=O)NC6=O)C7=O)ncn5)CC4)C3)cc2F)CC1] |
| 175 | 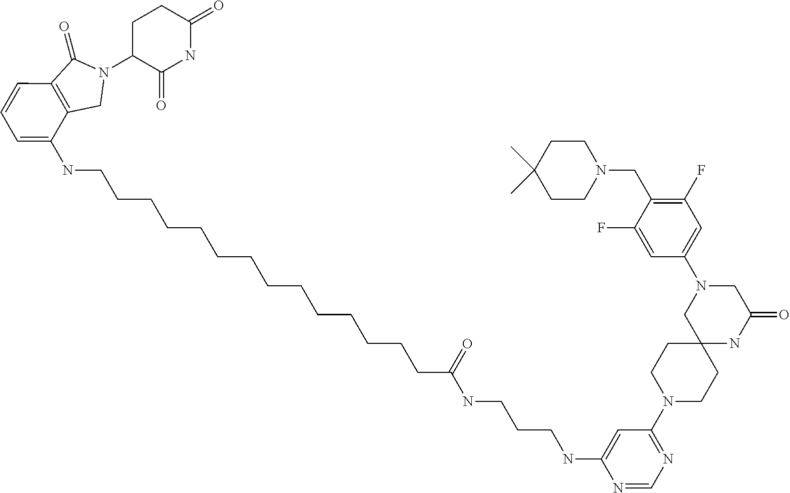 [SMILES: CC1(C)CCN(Cc2c(F)cc(N3CC(=O)NC4(CCN(c5cc(NCCCNC(=O)CCCCCCCCCCCCCCNc6cccc7c6CN(C6CCC(=O)NC6=O)C7=O)ncn5)CC4)C3)cc2F)CC1] |
| 176 | 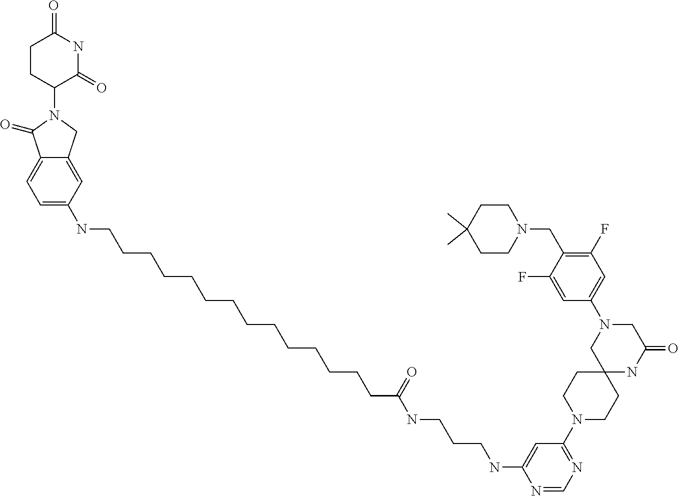 [SMILES: CC1(C)CCN(Cc2c(F)cc(N3CC(=O)NC4(CCN(c5cc(NCCCNC(=O)CCCCCCCCCCCCCCNc6ccc7c(c6)CN(C6CCC(=O)NC6=O)C7=O)ncn5)CC4)C3)cc2F)CC1] |
| 177 | 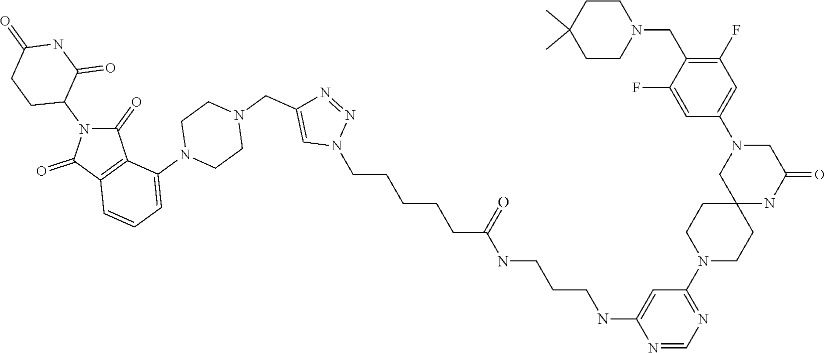 [SMILES: CC1(C)CCN(Cc2c(F)cc(N3CC(=O)NC4(CCN(c5cc(NCCCNC(=O)CCCCCn6cc(CN7CCN(c8cccc9c8C(=O)N(C8CCC(=O)NC8=O)C9=O)CC7)nn6)ncn5)CC4)C3)cc2F)CC1] |
| 178 | 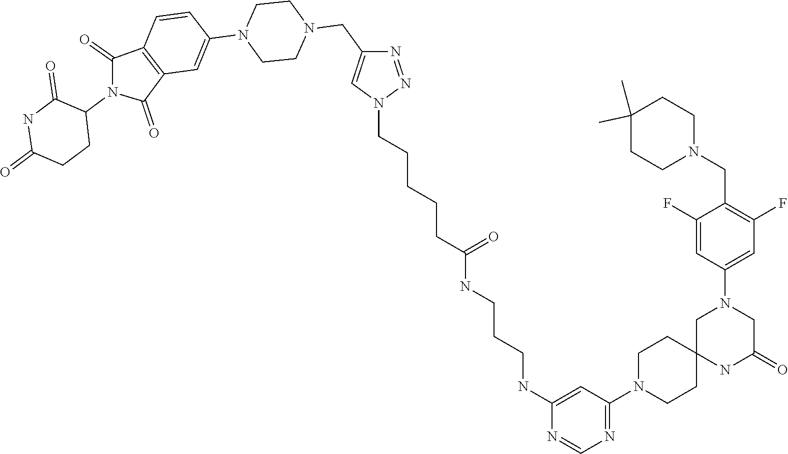 [SMILES: CC1(C)CCN(Cc2c(F)cc(N3CC(=O)NC4(CCN(c5cc(NCCCNC(=O)CCCCCn6cc(CN7CCN(c8ccc9c(c8)C(=O)N(C8CCC(=O)NC8=O)C9=O)CC7)nn6)ncn5)CC4)C3)cc2F)CC1] |
| 179 | 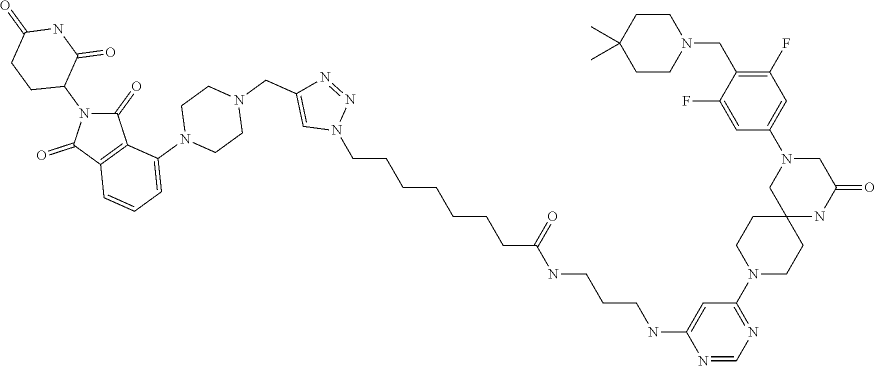 [SMILES: CC1(C)CCN(Cc2c(F)cc(N3CC(=O)NC4(CCN(c5cc(NCCCNC(=O)CCCCCCCn6cc(CN7CCN(c8cccc9c8C(=O)N(C8CCC(=O)NC8=O)C9=O)CC7)nn6)ncn5)CC4)C3)cc2F)CC1] |
| 180 | 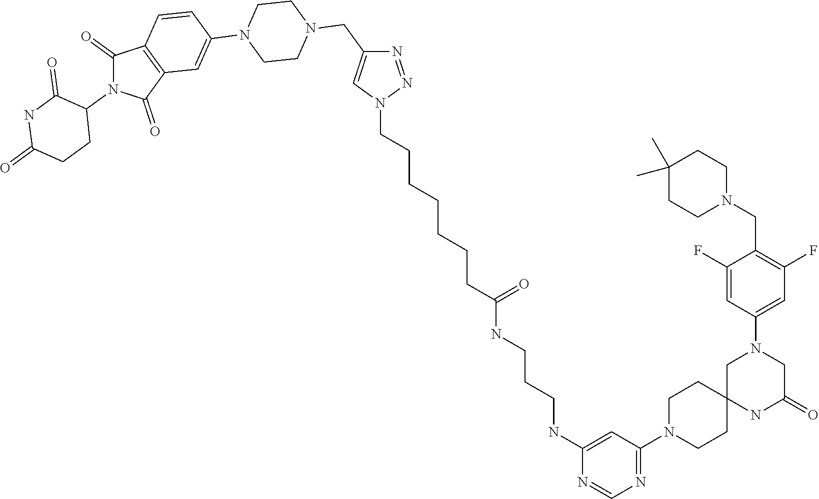 [SMILES: CC1(C)CCN(Cc2c(F)cc(N3CC(=O)NC4(CCN(c5cc(NCCCNC(=O)CCCCCCCn6cc(CN7CCN(c8ccc9c(c8)C(=O)N(C8CCC(=O)NC8=O)C9=O)CC7)nn6)ncn5)CC4)C3)cc2F)CC1] |
| 181 | 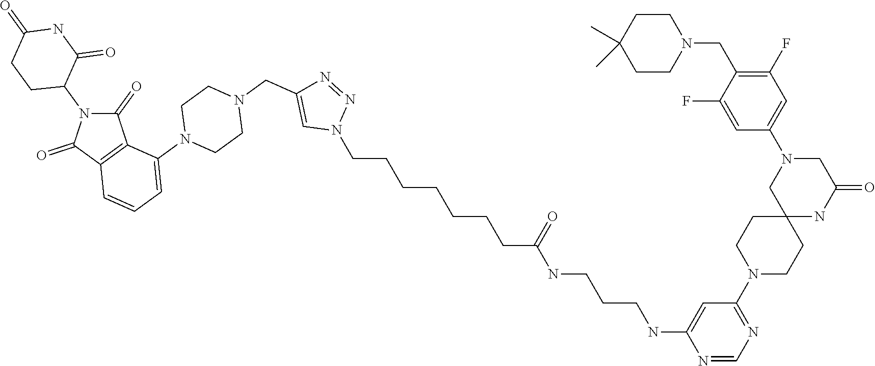 [SMILES: CC1(C)CCN(Cc2c(F)cc(N3CC(=O)NC4(CCN(c5cc(NCCCNC(=O)CCCCCCCn6cc(CN7CCN(c8cccc9c8C(=O)N(C8CCC(=O)NC8=O)C9=O)CC7)nn6)ncn5)CC4)C3)cc2F)CC1] |
| 182 | 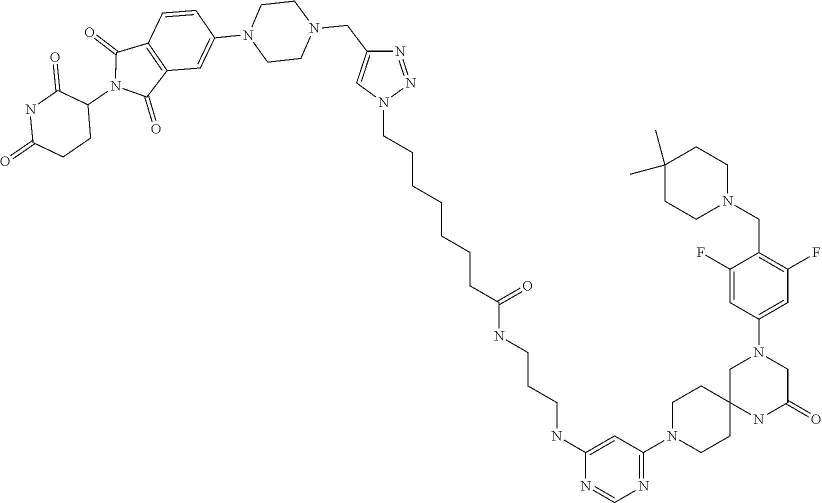 [SMILES: CC1(C)CCN(Cc2c(F)cc(N3CC(=O)NC4(CCN(c5cc(NCCCNC(=O)CCCCCCCn6cc(CN7CCN(c8ccc9c(c8)C(=O)N(C8CCC(=O)NC8=O)C9=O)CC7)nn6)ncn5)CC4)C3)cc2F)CC1] |
| 183 | 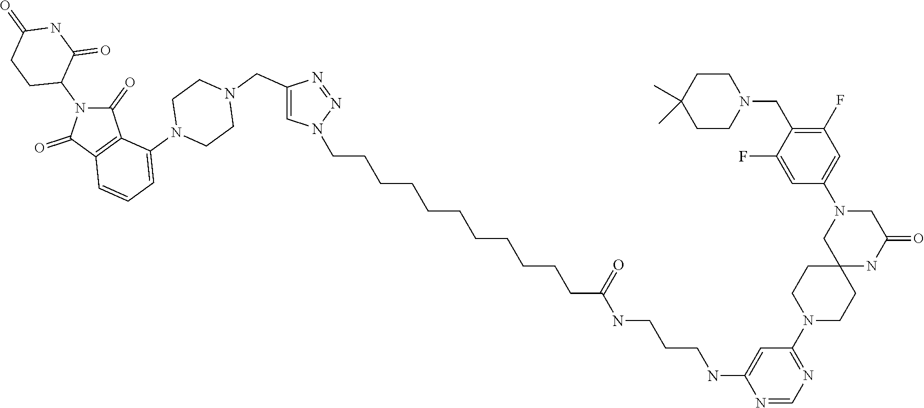 [SMILES: CC1(C)CCN(Cc2c(F)cc(N3CC(=O)NC4(CCN(c5cc(NCCCNC(=O)CCCCCCCCCCCn6cc(CN7CCN(c8cccc9c8C(=O)N(C8CCC(=O)NC8=O)C9=O)CC7)nn6)ncn5)CC4)C3)cc2F)CC1] |
| 184 | 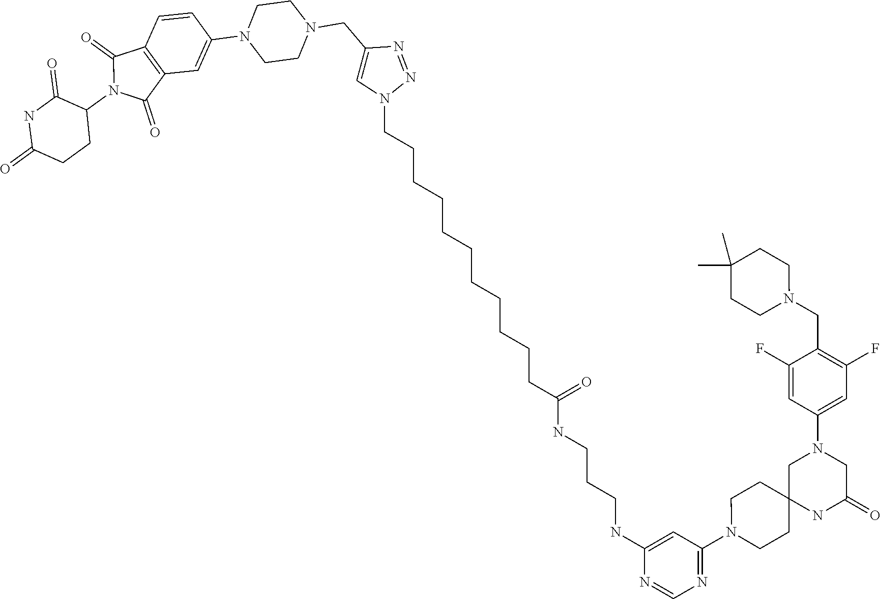 [SMILES: CC1(C)CCN(Cc2c(F)cc(N3CC(=O)NC4(CCN(c5cc(NCCCNC(=O)CCCCCCCCCCCn6cc(CN7CCN(c8ccc9c(c8)C(=O)N(C8CCC(=O)NC8=O)C9=O)CC7)nn6)ncn5)CC4)C3)cc2F)CC1] |
| 185 | 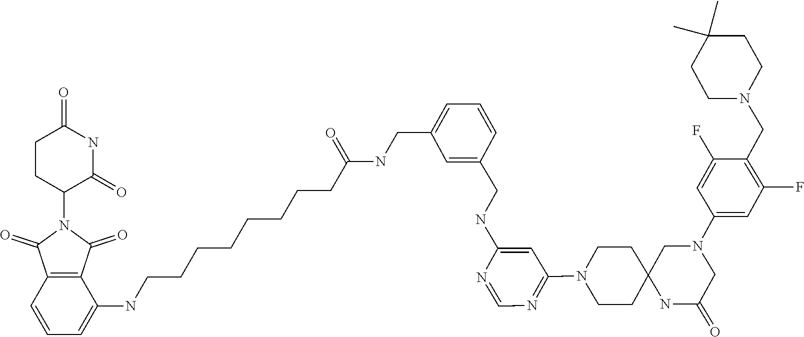 [SMILES: CC1(C)CCN(Cc2c(F)cc(N3CC(=O)NC4(CCN(c5cc(NCc6cccc(CNC(=O)CCCCCCCCNc7cccc8c7C(=O)N(C7CCC(=O)NC7=O)C8=O)c6)ncn5)CC4)C3)cc2F)CC1] |
| 186 | 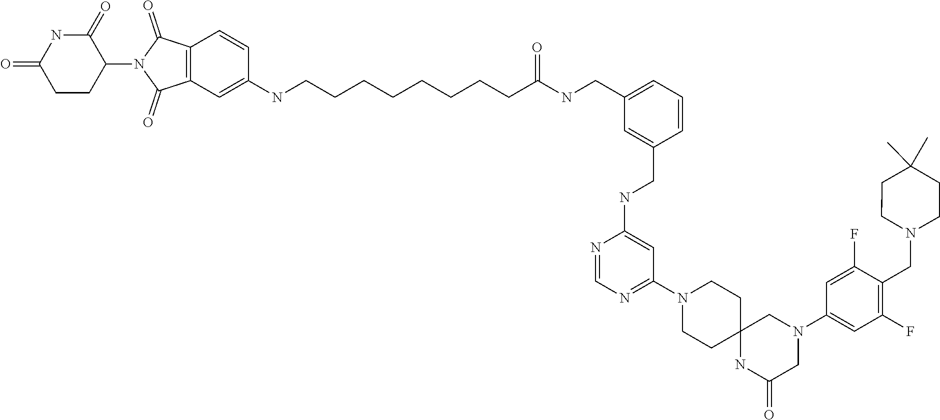 [SMILES: CC1(C)CCN(Cc2c(F)cc(N3CC(=O)NC4(CCN(c5cc(NCc6cccc(CNC(=O)CCCCCCCCNc7ccc8c(c7)C(=O)N(C7CCC(=O)NC7=O)C8=O)c6)ncn5)CC4)C3)cc2F)CC1] |
| 187 | 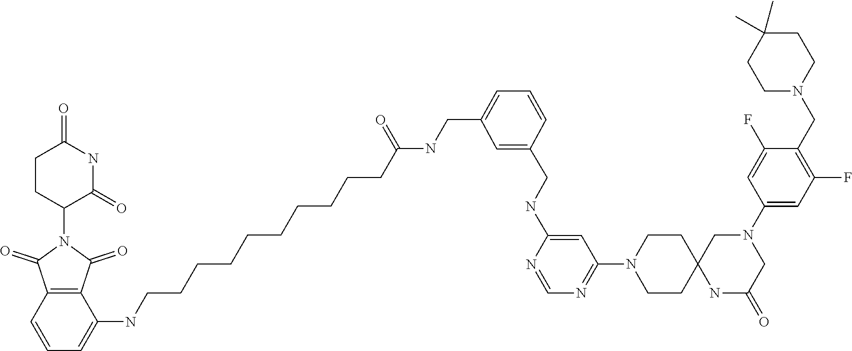 [SMILES: CC1(C)CCN(Cc2c(F)cc(N3CC(=O)NC4(CCN(c5cc(NCc6cccc(CNC(=O)CCCCCCCCCCNc7cccc8c7C(=O)N(C7CCC(=O)NC7=O)C8=O)c6)ncn5)CC4)C3)cc2F)CC1] |
| 188 | 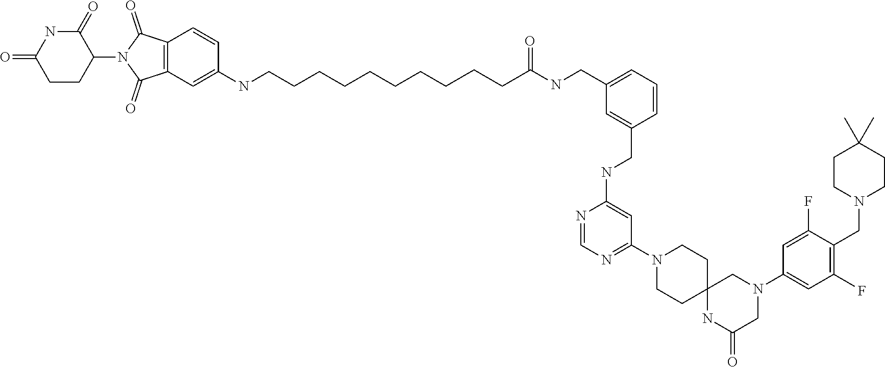 [SMILES: CC1(C)CCN(Cc2c(F)cc(N3CC(=O)NC4(CCN(c5cc(NCc6cccc(CNC(=O)CCCCCCCCCCNc7ccc8c(c7)C(=O)N(C7CCC(=O)NC7=O)C8=O)c6)ncn5)CC4)C3)cc2F)CC1] |
| 189 | 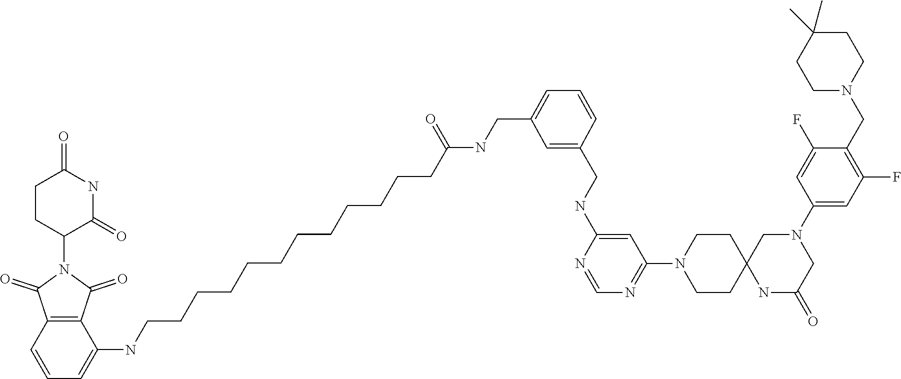 [SMILES: CC1(C)CCN(Cc2c(F)cc(N3CC(=O)NC4(CCN(c5cc(NCc6cccc(CNC(=O)CCCCCCCCCCCCNc7cccc8c7C(=O)N(C7CCC(=O)NC7=O)C8=O)c6)ncn5)CC4)C3)cc2F)CC1] |
| 190 | 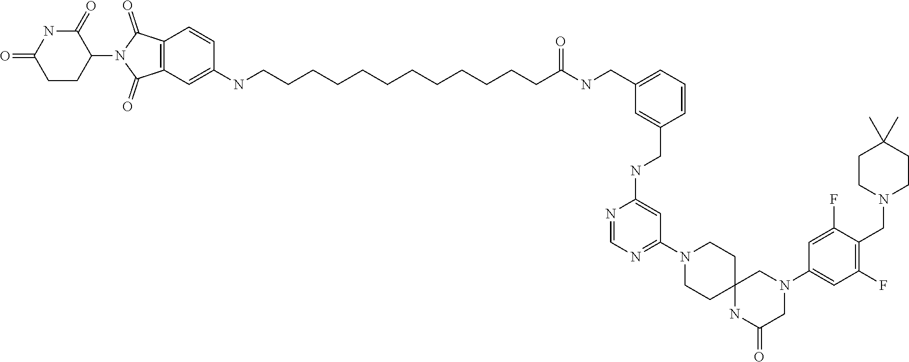 [SMILES: CC1(C)CCN(Cc2c(F)cc(N3CC(=O)NC4(CCN(c5cc(NCc6cccc(CNC(=O)CCCCCCCCCCCCNc7ccc8c(c7)C(=O)N(C7CCC(=O)NC7=O)C8=O)c6)ncn5)CC4)C3)cc2F)CC1] |
| 191 | 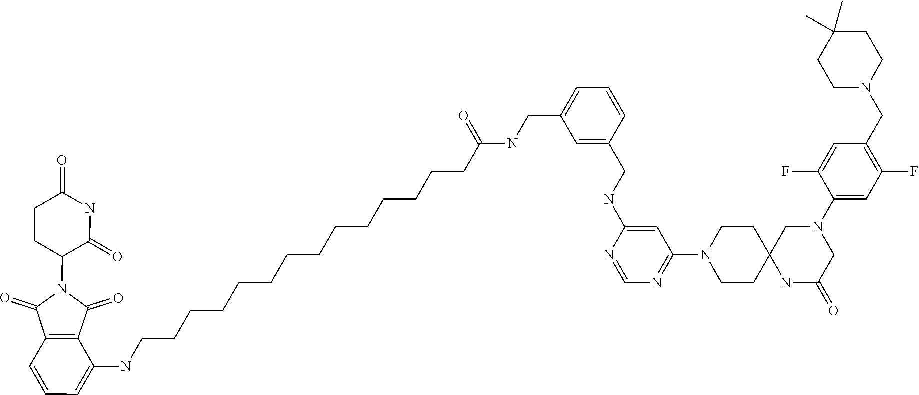 [SMILES: CC1(C)CCN(Cc2cc(F)c(N3CC(=O)NC4(CCN(c5cc(NCc6cccc(CNC(=O)CCCCCCCCCCCCCCNc7cccc8c7C(=O)N(C7CCC(=O)NC7=O)C8=O)c6)ncn5)CC4)C3)cc2F)CC1] |
| 192 | 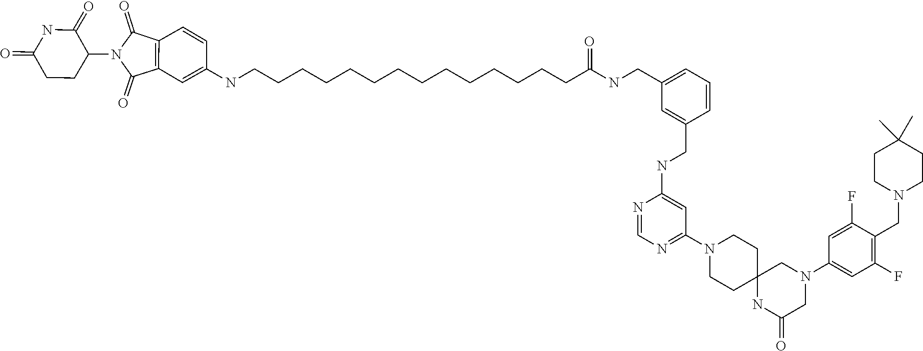 [SMILES: CC1(C)CCN(Cc2c(F)cc(N3CC(=O)NC4(CCN(c5cc(NCc6cccc(CNC(=O)CCCCCCCCCCCCCCNc7ccc8c(c7)C(=O)N(C7CCC(=O)NC7=O)C8=O)c6)ncn5)CC4)C3)cc2F)CC1] |
| 193 | 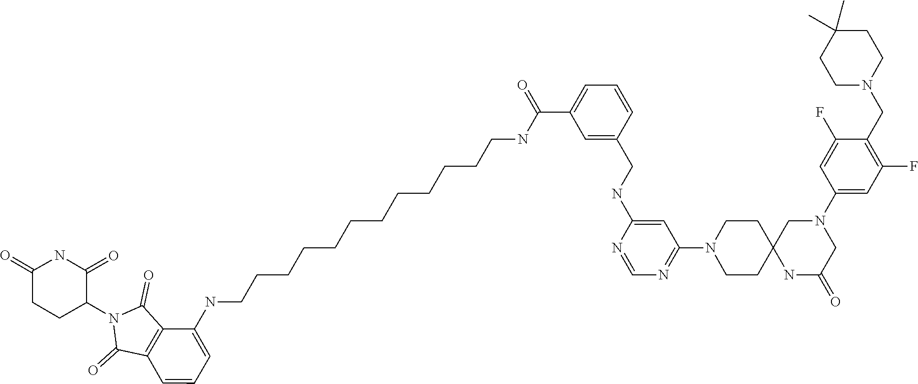 [SMILES: CC1(C)CCN(Cc2c(F)cc(N3CC(=O)NC4(CCN(c5cc(NCc6cccc(C(=O)NCCCCCCCCCCCCNc7cccc8c7C(=O)N(C7CCC(=O)NC7=O)C8=O)c6)ncn5)CC4)C3)cc2F)CC1] |
| 194 | 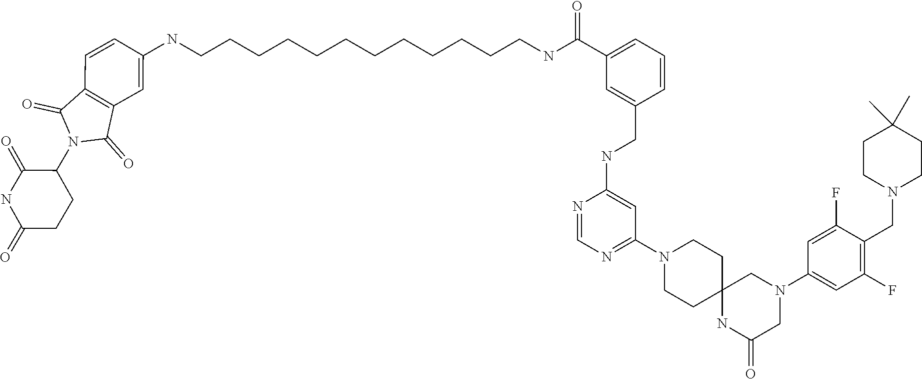 [SMILES: CC1(C)CCN(Cc2c(F)cc(N3CC(=O)NC4(CCN(c5cc(NCc6cccc(C(=O)NCCCCCCCCCCCCNc7ccc8c(c7)C(=O)N(C7CCC(=O)NC7=O)C8=O)c6)ncn5)CC4)C3)cc2F)CC1] |
| 195 | 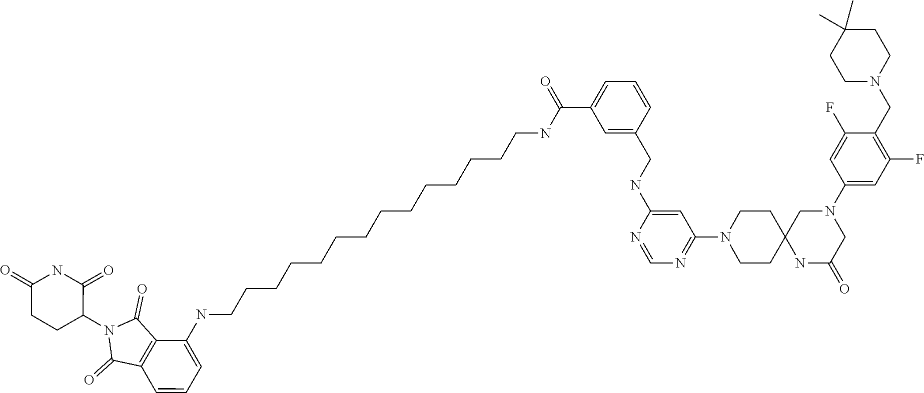 [SMILES: CC1(C)CCN(Cc2c(F)cc(N3CC(=O)NC4(CCN(c5cc(NCc6cccc(C(=O)NCCCCCCCCCCCCCCNc7cccc8c7C(=O)N(C7CCC(=O)NC7=O)C8=O)c6)ncn5)CC4)C3)cc2F)CC1] |
| 196 | 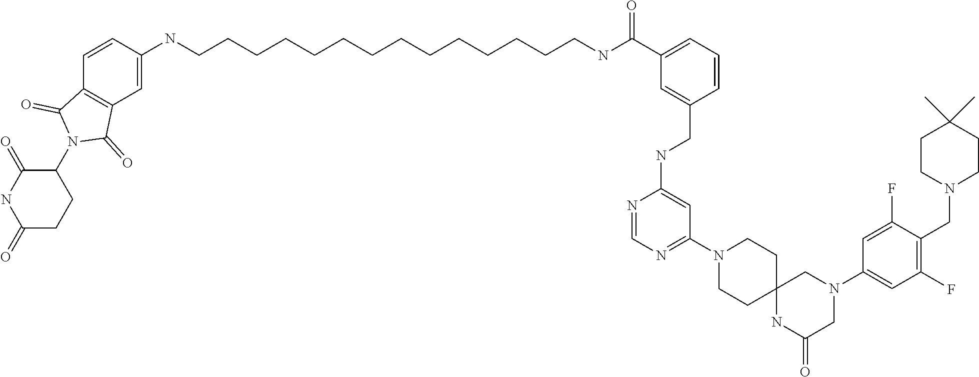 [SMILES: CC1(C)CCN(Cc2c(F)cc(N3CC(=O)NC4(CCN(c5cc(NCc6cccc(C(=O)NCCCCCCCCCCCCCCNc7ccc8c(c7)C(=O)N(C7CCC(=O)NC7=O)C8=O)c6)ncn5)CC4)C3)cc2F)CC1] |
| 197 | 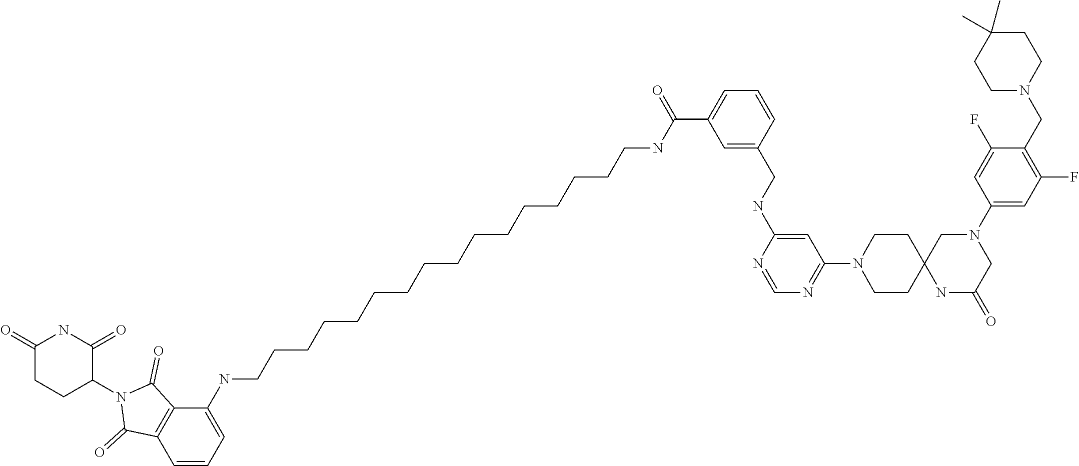 [SMILES: CC1(C)CCN(Cc2c(F)cc(N3CC(=O)NC4(CCN(c5cc(NCc6cccc(C(=O)NCCCCCCCCCCCCCCCCNc7cccc8c7C(=O)N(C7CCC(=O)NC7=O)C8=O)c6)ncn5)CC4)C3)cc2F)CC1] |
| 198 | 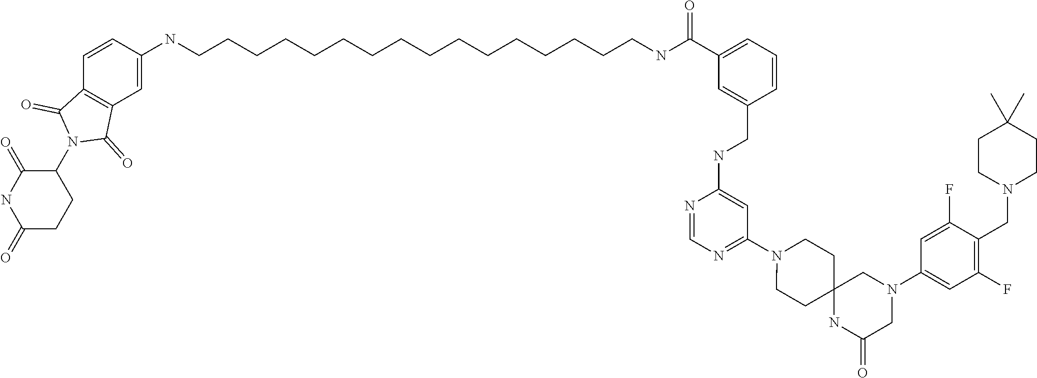 [SMILES: CC1(C)CCN(Cc2c(F)cc(N3CC(=O)NC4(CCN(c5cc(NCc6cccc(C(=O)NCCCCCCCCCCCCCCCCNc7ccc8c(c7)C(=O)N(C7CCC(=O)NC7=O)C8=O)c6)ncn5)CC4)C3)cc2F)CC1] |
| 199 | 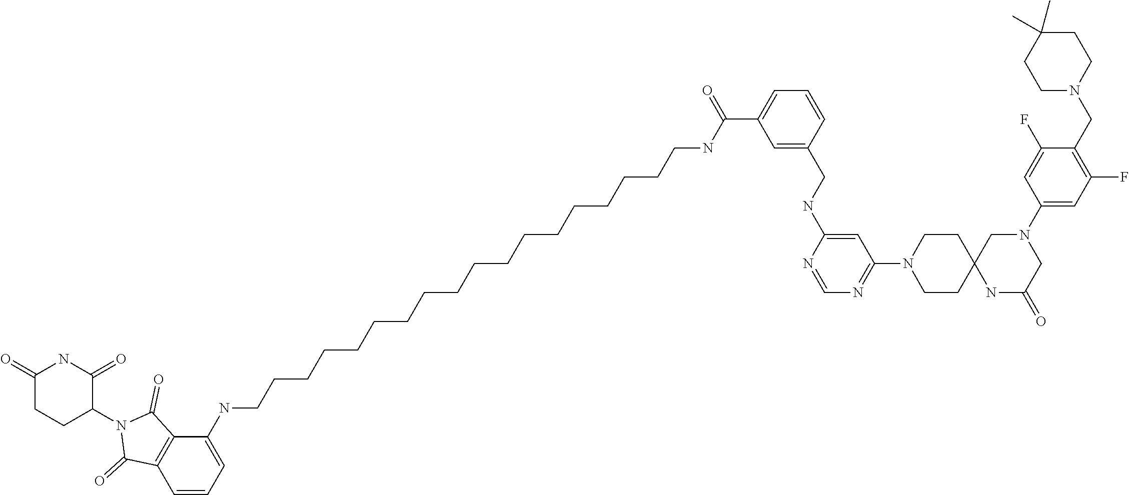 [SMILES: CC1(C)CCN(Cc2c(F)cc(N3CC(=O)NC4(CCN(c5cc(NCc6cccc(C(=O)NCCCCCCCCCCCCCCCCCCNc7cccc8c7C(=O)N(C7CCC(=O)NC7=O)C8=O)c6)ncn5)CC4)C3)cc2F)CC1] |
| 200 | 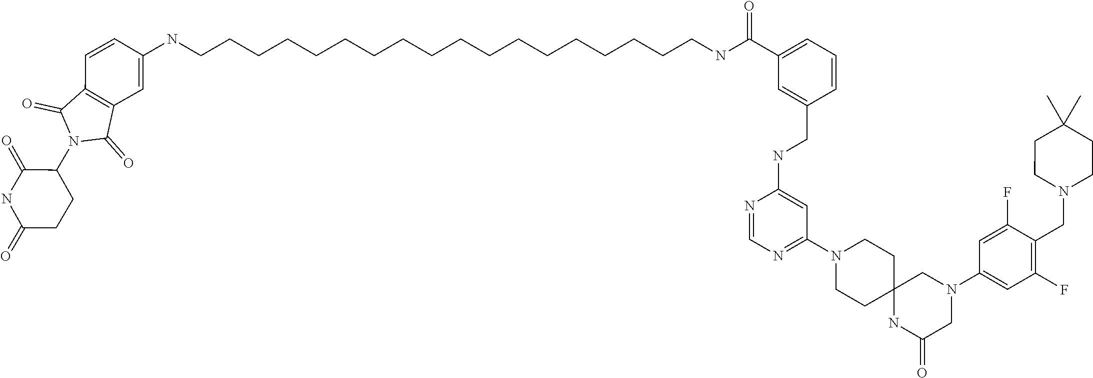 [SMILES: CC1(C)CCN(Cc2c(F)cc(N3CC(=O)NC4(CCN(c5cc(NCc6cccc(C(=O)NCCCCCCCCCCCCCCCCCCNc7ccc8c(c7)C(=O)N(C7CCC(=O)NC7=O)C8=O)c6)ncn5)CC4)C3)cc2F)CC1] |
| 201 | 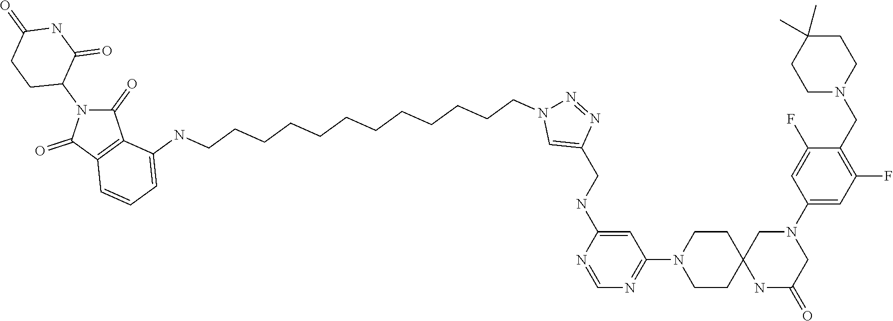 [SMILES: CC1(C)CCN(Cc2c(F)cc(N3CC(=O)NC4(CCN(c5cc(NCc6cn(CCCCCCCCCCCCNc7cccc8c7C(=O)N(C7CCC(=O)NC7=O)C8=O)nn6)ncn5)CC4)C3)cc2F)CC1] |
| 202 | 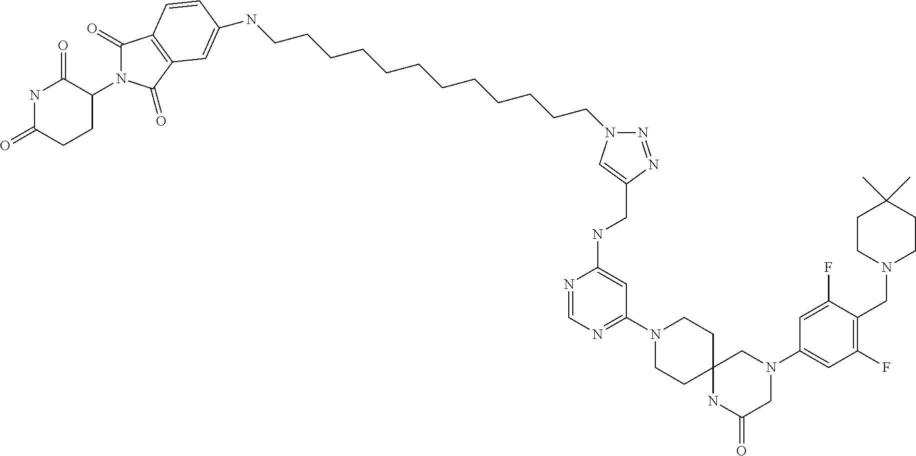 [SMILES: CC1(C)CCN(Cc2c(F)cc(N3CC(=O)NC4(CCN(c5cc(NCc6cn(CCCCCCCCCCCCNc7ccc8c(c7)C(=O)N(C7CCC(=O)NC7=O)C8=O)nn6)ncn5)CC4)C3)cc2F)CC1] |
| 203 | 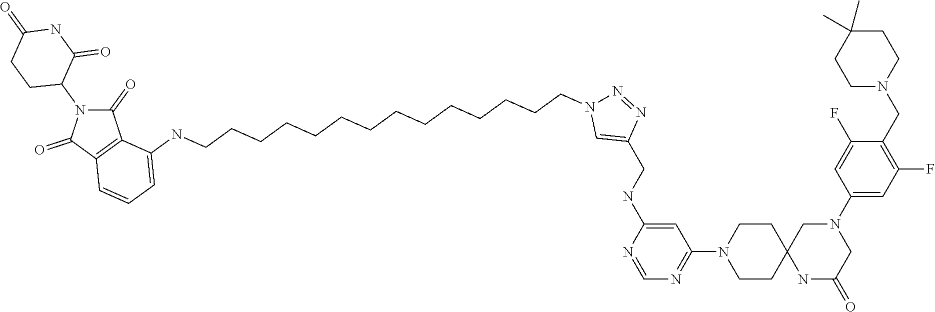 [SMILES: CC1(C)CCN(Cc2c(F)cc(N3CC(=O)NC4(CCN(c5cc(NCc6cn(CCCCCCCCCCCCCCNc7cccc8c7C(=O)N(C7CCC(=O)NC7=O)C8=O)nn6)ncn5)CC4)C3)cc2F)CC1] |
| 204 | 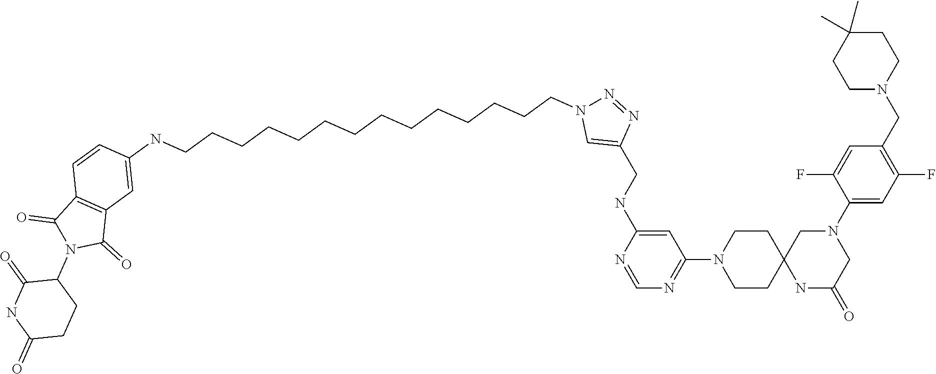 [SMILES: CC1(C)CCN(Cc2cc(F)c(N3CC(=O)NC4(CCN(c5cc(NCc6cn(CCCCCCCCCCCCCCNc7ccc8c(c7)C(=O)N(C7CCC(=O)NC7=O)C8=O)nn6)ncn5)CC4)C3)cc2F)CC1] |
| 205 | 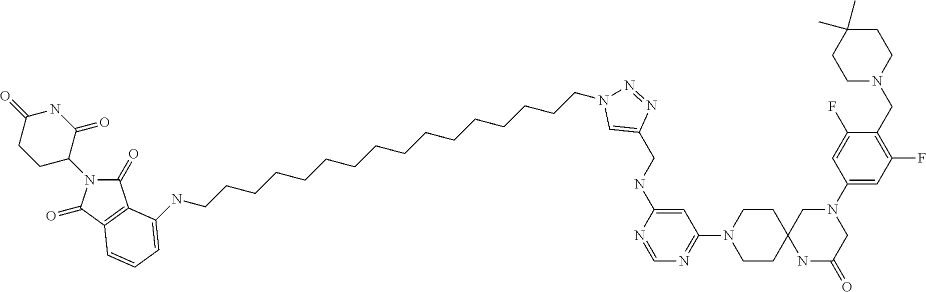 [SMILES: CC1(C)CCN(Cc2c(F)cc(N3CC(=O)NC4(CCN(c5cc(NCc6cn(CCCCCCCCCCCCCCCCNc7cccc8c7C(=O)N(C7CCC(=O)NC7=O)C8=O)nn6)ncn5)CC4)C3)cc2F)CC1] |
| 206 | 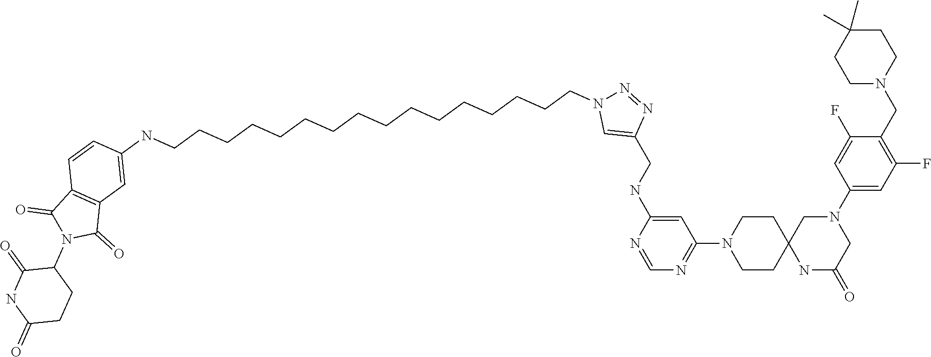 [SMILES: CC1(C)CCN(Cc2c(F)cc(N3CC(=O)NC4(CCN(c5cc(NCc6cn(CCCCCCCCCCCCCCCCNc7ccc8c(c7)C(=O)N(C7CCC(=O)NC7=O)C8=O)nn6)ncn5)CC4)C3)cc2F)CC1] |
| 207 | 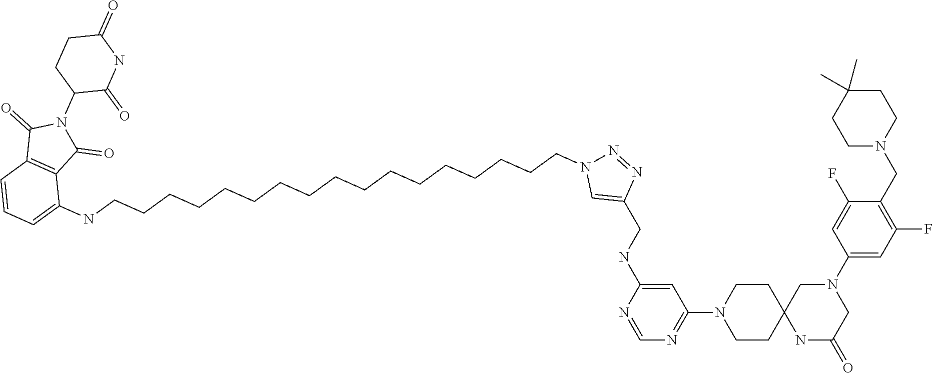 [SMILES: CC1(C)CCN(Cc2c(F)cc(N3CC(=O)NC4(CCN(c5cc(NCc6cn(CCCCCCCCCCCCCCCCCNc7cccc8c7C(=O)N(C7CCC(=O)NC7=O)C8=O)nn6)ncn5)CC4)C3)cc2F)CC1] |
| 208 | 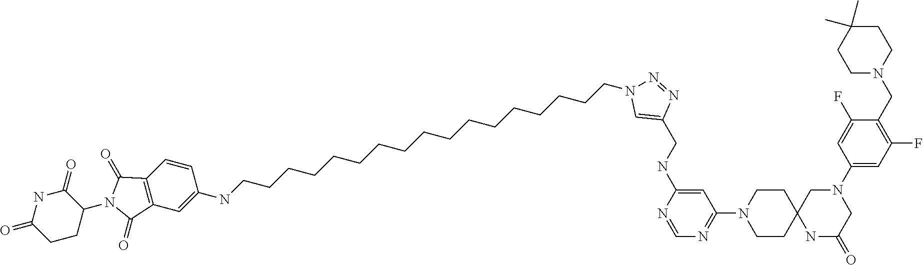 [SMILES: CC1(C)CCN(Cc2c(F)cc(N3CC(=O)NC4(CCN(c5cc(NCc6cn(CCCCCCCCCCCCCCCCCNc7ccc8c(c7)C(=O)N(C7CCC(=O)NC7=O)C8=O)nn6)ncn5)CC4)C3)cc2F)CC1] |
| 210 | 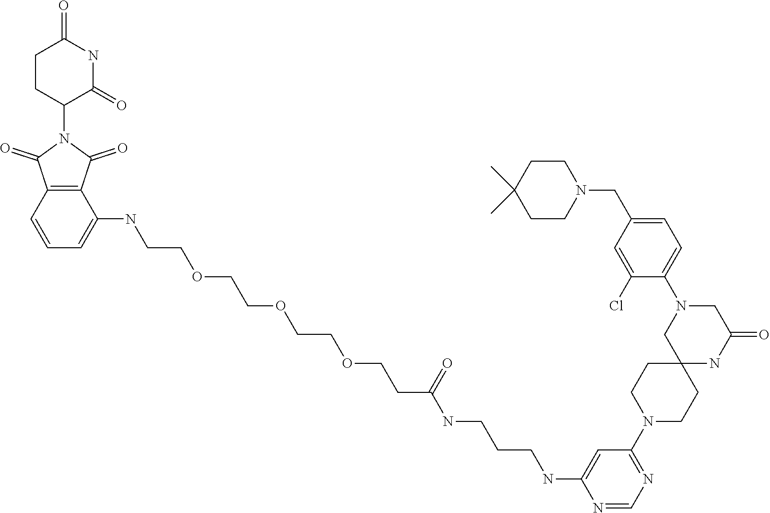 [SMILES: CC1(C)CCN(Cc2ccc(N3CC(=O)NC4(CCN(c5cc(NCCCNC(=O)CCOCCOCCOCCNc6cccc7c6C(=O)N(C6CCC(=O)NC6=O)C7=O)ncn5)CC4)C3)c(Cl)c2)CC1] |
| 211 | 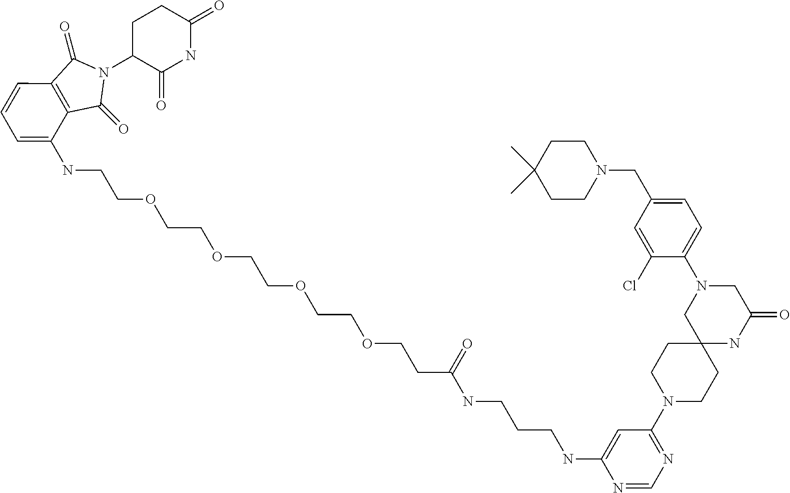 [SMILES: CC1(C)CCN(Cc2ccc(N3CC(=O)NC4(CCN(c5cc(NCCCNC(=O)CCOCCOCCOCCOCCNc6cccc7c6C(=O)N(C6CCC(=O)NC6=O)C7=O)ncn5)CC4)C3)c(Cl)c2)CC1] |
| 211 | 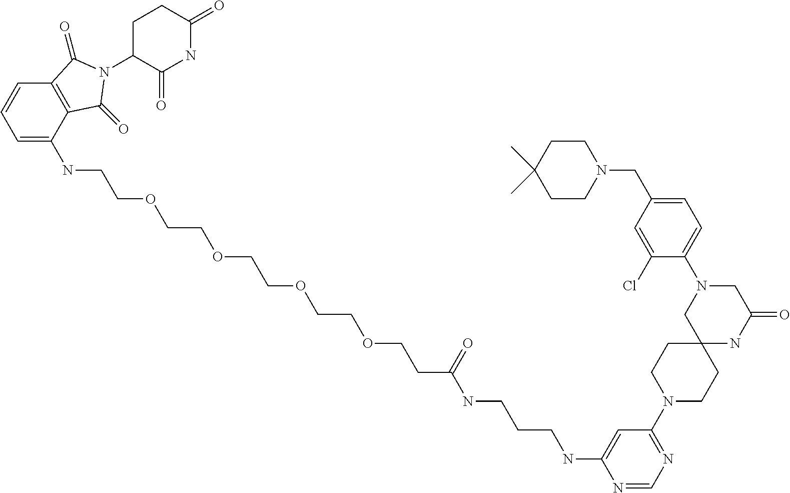 [SMILES: CC1(C)CCN(Cc2ccc(N3CC(=O)NC4(CCN(c5cc(NCCCNC(=O)CCOCCOCCOCCOCCNc6cccc7c6C(=O)N(C6CCC(=O)NC6=O)C7=O)ncn5)CC4)C3)c(Cl)c2)CC1] |
| 212 | 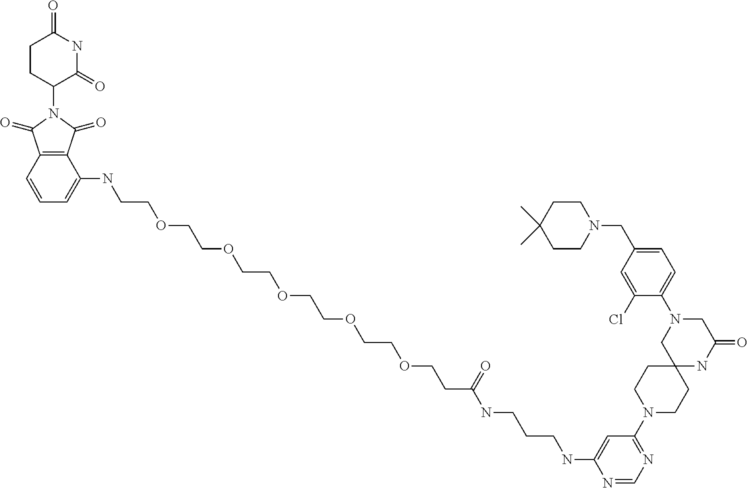 [SMILES: CC1(C)CCN(Cc2ccc(N3CC(=O)NC4(CCN(c5cc(NCCCNC(=O)CCOCCOCCOCCOCCOCCNc6cccc7c6C(=O)N(C6CCC(=O)NC6=O)C7=O)ncn5)CC4)C3)c(Cl)c2)CC1] |
| 213 | 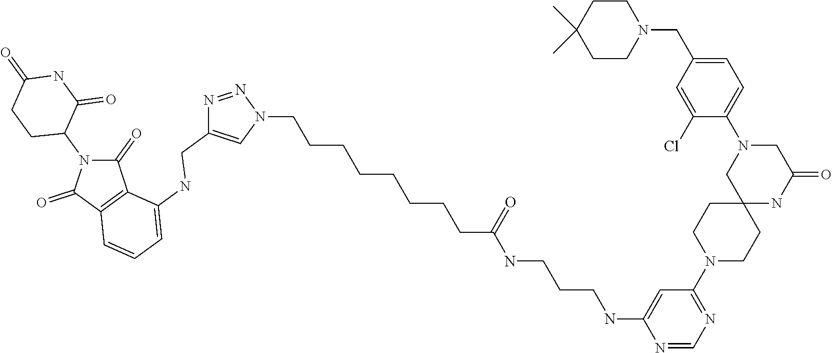 [SMILES: CC1(C)CCN(Cc2ccc(N3CC(=O)NC4(CCN(c5cc(NCCCNC(=O)CCCCCCCCn6cc(CNc7cccc8c7C(=O)N(C7CCC(=O)NC7=O)C8=O)nn6)ncn5)CC4)C3)c(Cl)c2)CC1] |
| 214 | 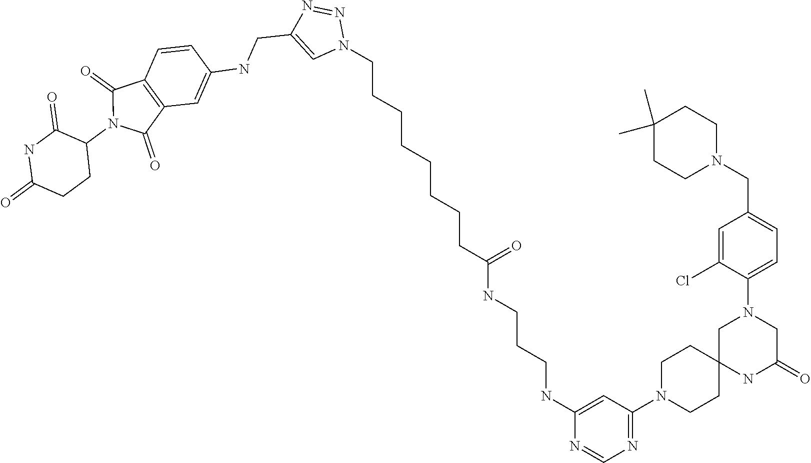 [SMILES: CC1(C)CCN(Cc2ccc(N3CC(=O)NC4(CCN(c5cc(NCCCNC(=O)CCCCCCCCn6cc(CNc7ccc8c(c7)C(=O)N(C7CCC(=O)NC7=O)C8=O)nn6)ncn5)CC4)C3)c(Cl)c2)CC1] |
| 215 | 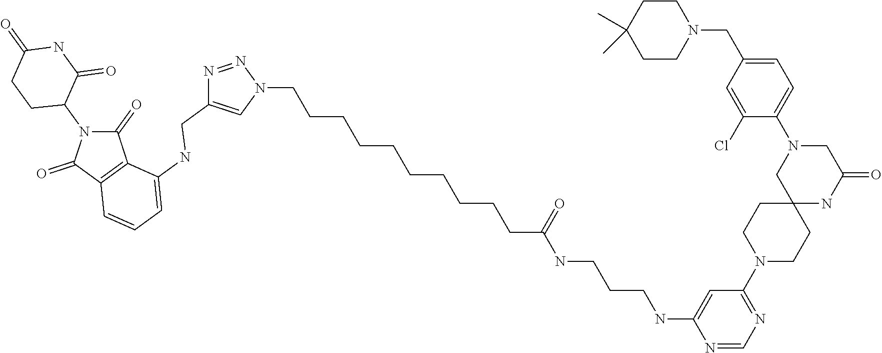 [SMILES: CC1(C)CCN(Cc2ccc(N3CC(=O)NC4(CCN(c5cc(NCCCNC(=O)CCCCCCCCCCn6cc(CNc7cccc8c7C(=O)N(C7CCC(=O)NC7=O)C8=O)nn6)ncn5)CC4)C3)c(Cl)c2)CC1] |
| 216 | 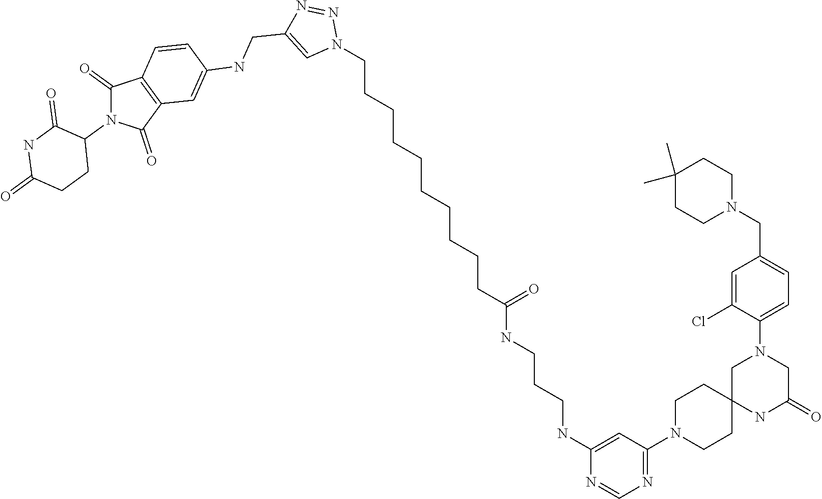 [SMILES: CC1(C)CCN(Cc2ccc(N3CC(=O)NC4(CCN(c5cc(NCCCNC(=O)CCCCCCCCCCn6cc(CNc7ccc8c(c7)C(=O)N(C7CCC(=O)NC7=O)C8=O)nn6)ncn5)CC4)C3)c(Cl)c2)CC1] |
| 217 | 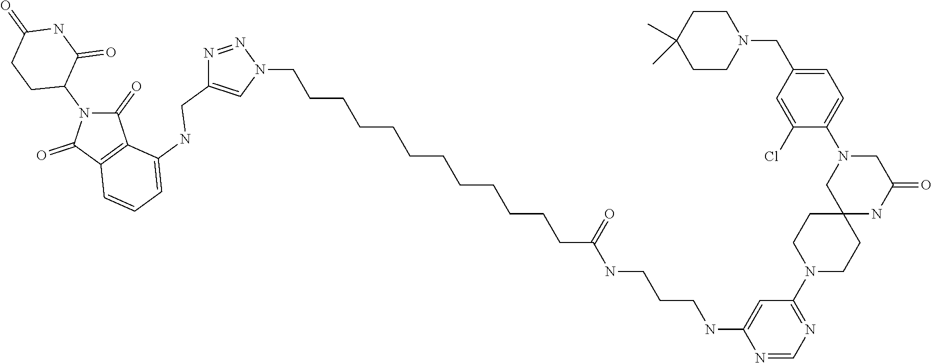 [SMILES: CC1(C)CCN(Cc2ccc(N3CC(=O)NC4(CCN(c5cc(NCCCNC(=O)CCCCCCCCCCCCn6cc(CNc7cccc8c7C(=O)N(C7CCC(=O)NC7=O)C8=O)nn6)ncn5)CC4)C3)c(Cl)c2)CC1] |
| 218 | 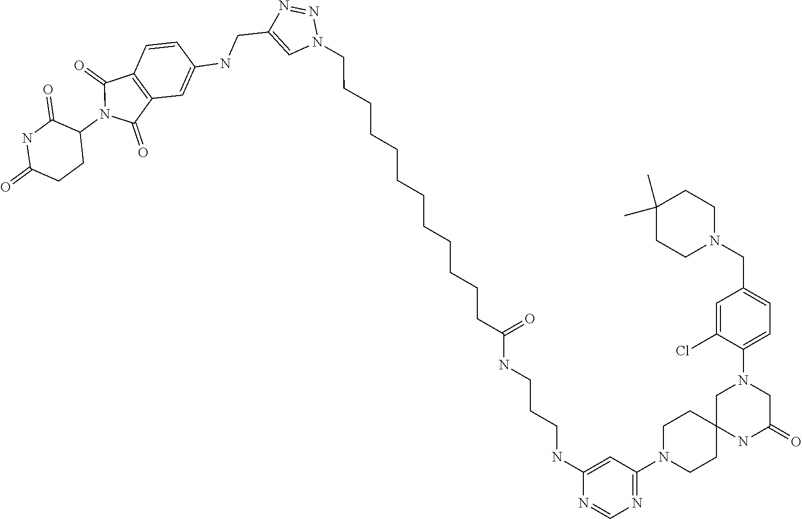 [SMILES: CC1(C)CCN(Cc2ccc(N3CC(=O)NC4(CCN(c5cc(NCCCNC(=O)CCCCCCCCCCCCn6cc(CNc7ccc8c(c7)C(=O)N(C7CCC(=O)NC7=O)C8=O)nn6)ncn5)CC4)C3)c(Cl)c2)CC1] |
| 219 | 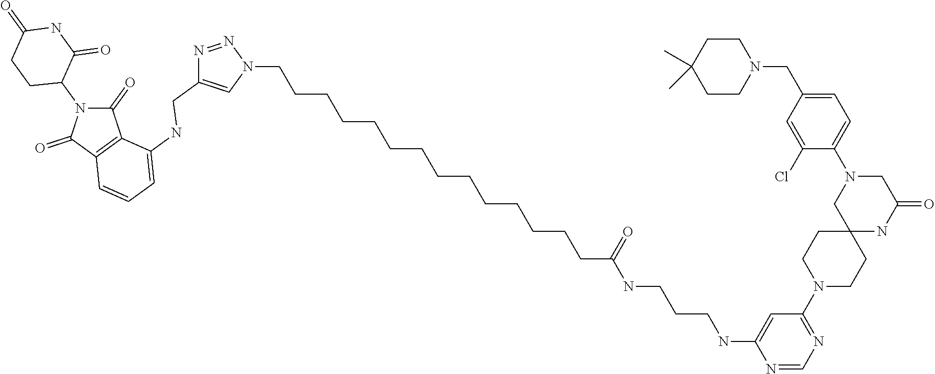 [SMILES: CC1(C)CCN(Cc2ccc(N3CC(=O)NC4(CCN(c5cc(NCCCNC(=O)CCCCCCCCCCCCCCn6cc(CNc7cccc8c7C(=O)N(C7CCC(=O)NC7=O)C8=O)nn6)ncn5)CC4)C3)c(Cl)c2)CC1] |
| 220 | 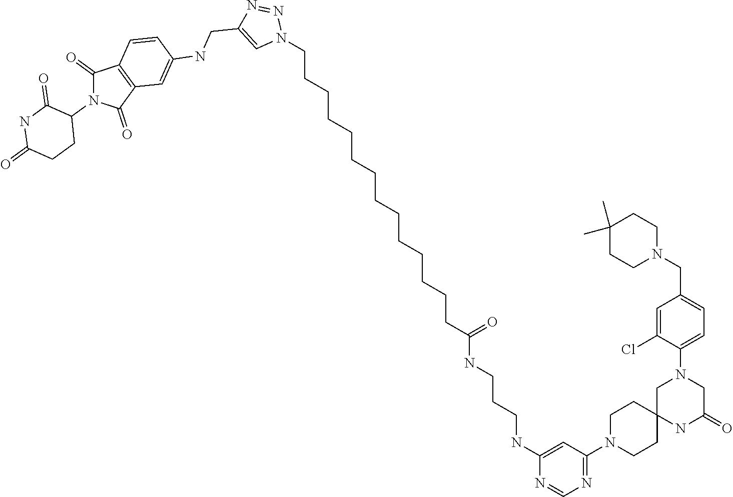 [SMILES: CC1(C)CCN(Cc2ccc(N3CC(=O)NC4(CCN(c5cc(NCCCNC(=O)CCCCCCCCCCCCCCn6cc(CNc7ccc8c(c7)C(=O)N(C7CCC(=O)NC7=O)C8=O)nn6)ncn5)CC4)C3)c(Cl)c2)CC1] |
| 220 | 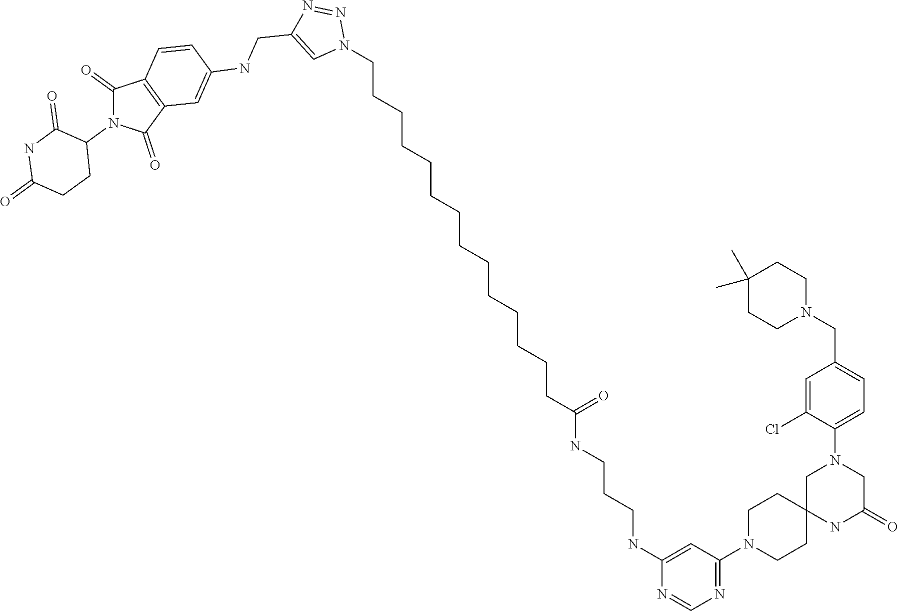 [SMILES: CC1(C)CCN(Cc2ccc(N3CC(=O)NC4(CCN(c5cc(NCCCNC(=O)CCCCCCCCCCCCCCn6cc(CNc7ccc8c(c7)C(=O)N(C7CCC(=O)NC7=O)C8=O)nn6)ncn5)CC4)C3)c(Cl)c2)CC1] |
| 221 | 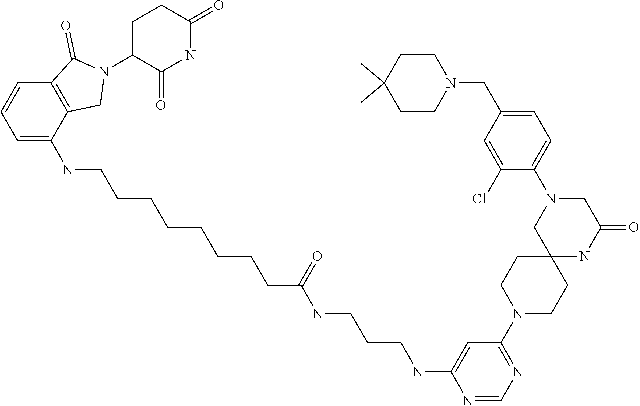 [SMILES: CC1(C)CCN(Cc2ccc(N3CC(=O)NC4(CCN(c5cc(NCCCNC(=O)CCCCCCCCNc6cccc7c6CN(C6CCC(=O)NC6=O)C7=O)ncn5)CC4)C3)c(Cl)c2)CC1] |
| 222 | 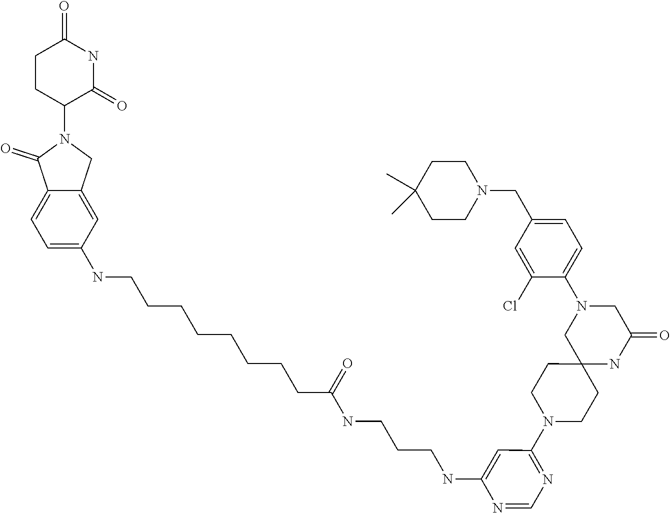 [SMILES: CC1(C)CCN(Cc2ccc(N3CC(=O)NC4(CCN(c5cc(NCCCNC(=O)CCCCCCCCNc6ccc7c(c6)CN(C6CCC(=O)NC6=O)C7=O)ncn5)CC4)C3)c(Cl)c2)CC1] |
| 223 | 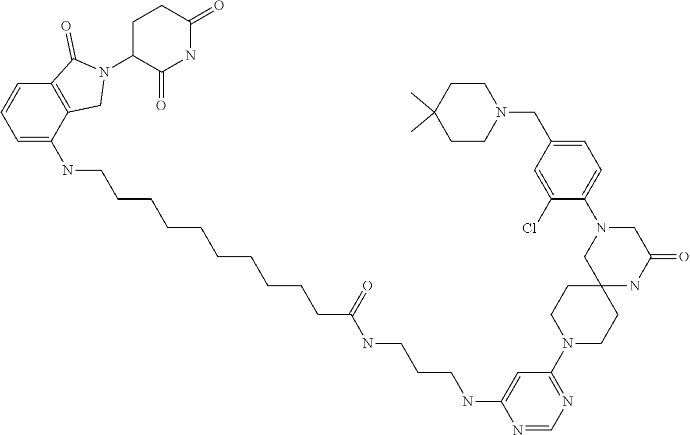 [SMILES: CC1(C)CCN(Cc2ccc(N3CC(=O)NC4(CCN(c5cc(NCCCNC(=O)CCCCCCCCCCNc6cccc7c6CN(C6CCC(=O)NC6=O)C7=O)ncn5)CC4)C3)c(Cl)c2)CC1] |
| 224 | 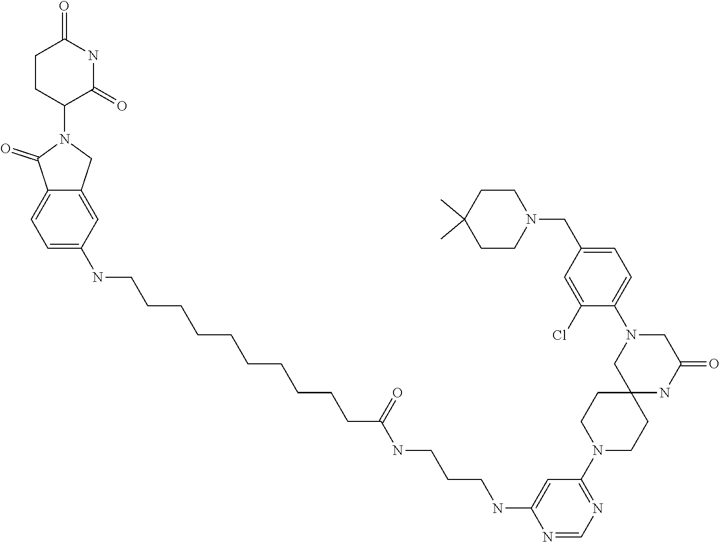 [SMILES: CC1(C)CCN(Cc2ccc(N3CC(=O)NC4(CCN(c5cc(NCCCNC(=O)CCCCCCCCCCNc6ccc7c(c6)CN(C6CCC(=O)NC6=O)C7=O)ncn5)CC4)C3)c(Cl)c2)CC1] |
| 225 | 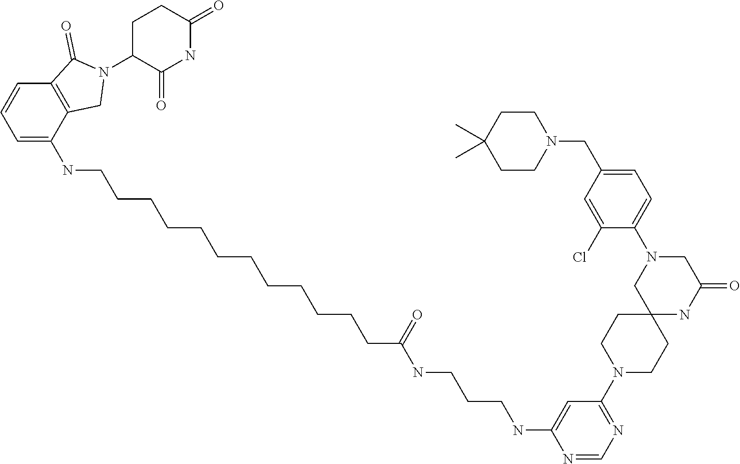 [SMILES: CC1(C)CCN(Cc2ccc(N3CC(=O)NC4(CCN(c5cc(NCCCNC(=O)CCCCCCCCCCCCNc6cccc7c6CN(C6CCC(=O)NC6=O)C7=O)ncn5)CC4)C3)c(Cl)c2)CC1] |
| 226 | 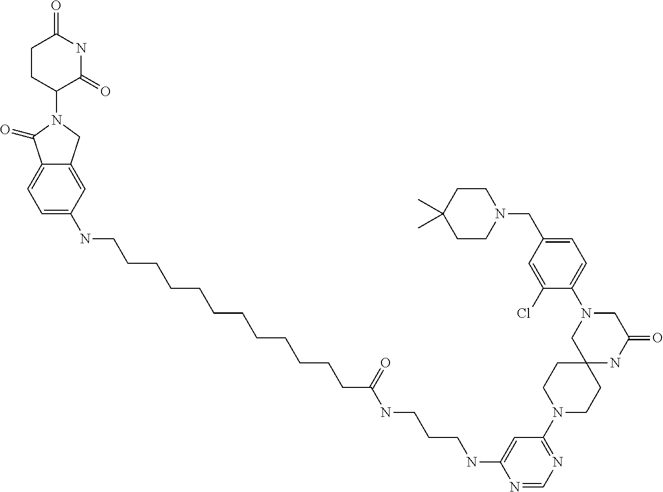 [SMILES: CC1(C)CCN(Cc2ccc(N3CC(=O)NC4(CCN(c5cc(NCCCNC(=O)CCCCCCCCCCCCNc6ccc7c(c6)CN(C6CCC(=O)NC6=O)C7=O)ncn5)CC4)C3)c(Cl)c2)CC1] |
| 227 | 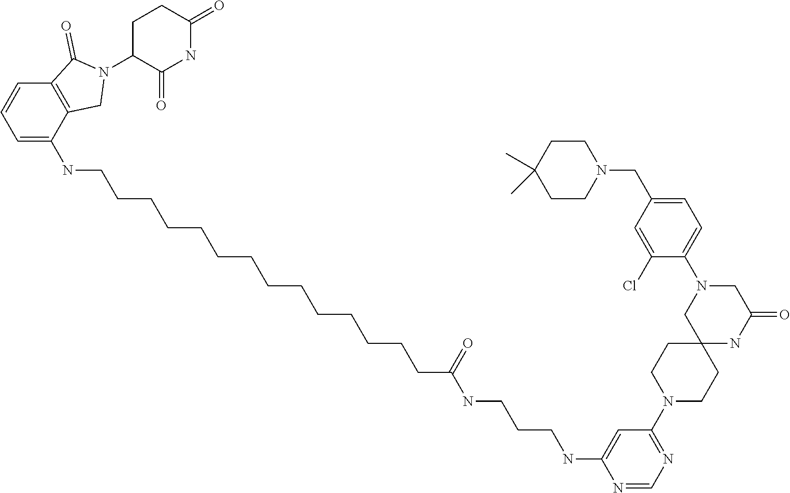 [SMILES: CC1(C)CCN(Cc2ccc(N3CC(=O)NC4(CCN(c5cc(NCCCNC(=O)CCCCCCCCCCCCCCNc6cccc7c6CN(C6CCC(=O)NC6=O)C7=O)ncn5)CC4)C3)c(Cl)c2)CC1] |
| 228 | 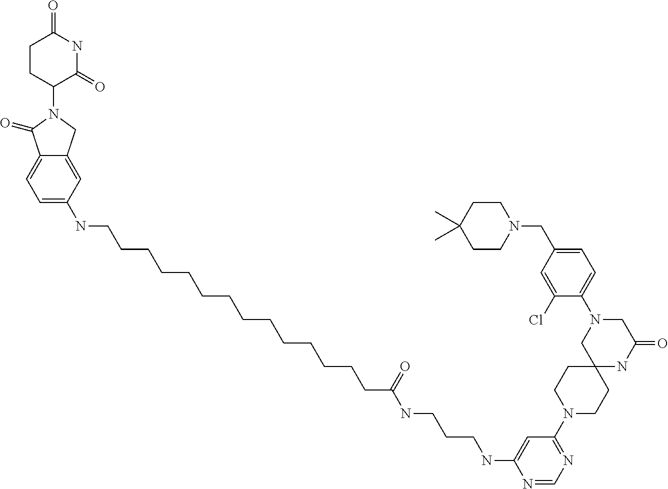 [SMILES: CC1(C)CCN(Cc2ccc(N3CC(=O)NC4(CCN(c5cc(NCCCNC(=O)CCCCCCCCCCCCCCNc6ccc7c(c6)CN(C6CCC(=O)NC6=O)C7=O)ncn5)CC4)C3)c(Cl)c2)CC1] |
| 229 | 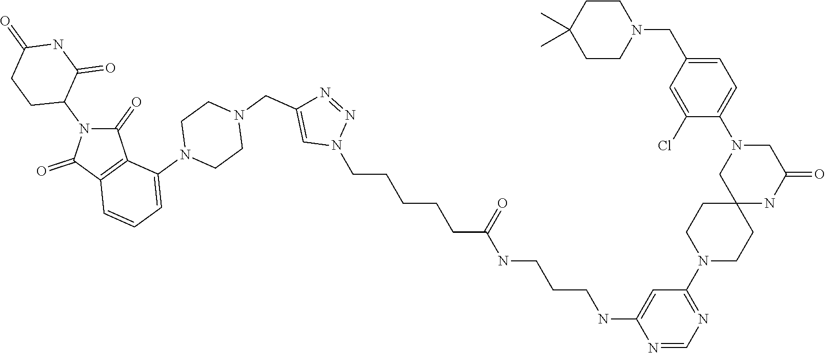 [SMILES: CC1(C)CCN(Cc2ccc(N3CC(=O)NC4(CCN(c5cc(NCCCNC(=O)CCCCCn6cc(CN7CCN(c8cccc9c8C(=O)N(C8CCC(=O)NC8=O)C9=O)CC7)nn6)ncn5)CC4)C3)c(Cl)c2)CC1] |
| 230 | 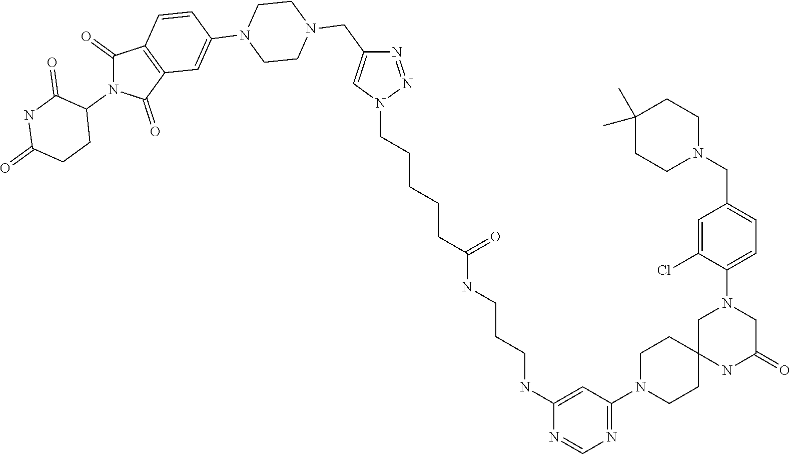 [SMILES: CC1(C)CCN(Cc2ccc(N3CC(=O)NC4(CCN(c5cc(NCCCNC(=O)CCCCCn6cc(CN7CCN(c8ccc9c(c8)C(=O)N(C8CCC(=O)NC8=O)C9=O)CC7)nn6)ncn5)CC4)C3)c(Cl)c2)CC1] |
| 231 | 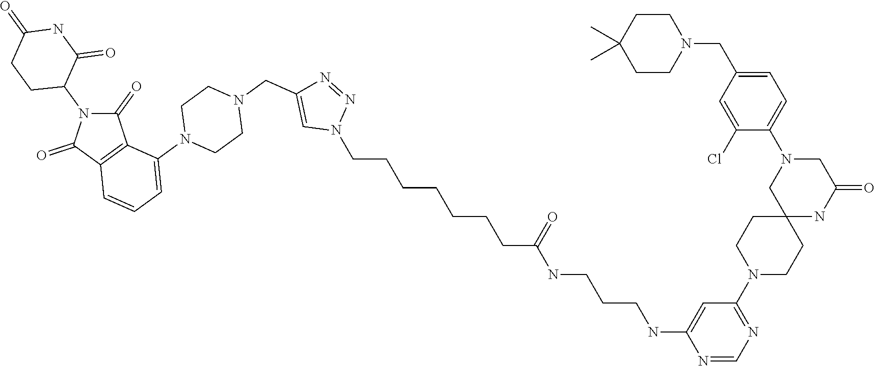 [SMILES: CC1(C)CCN(Cc2ccc(N3CC(=O)NC4(CCN(c5cc(NCCCNC(=O)CCCCCCCn6cc(CN7CCN(c8cccc9c8C(=O)N(C8CCC(=O)NC8=O)C9=O)CC7)nn6)ncn5)CC4)C3)c(Cl)c2)CC1] |
| 232 | 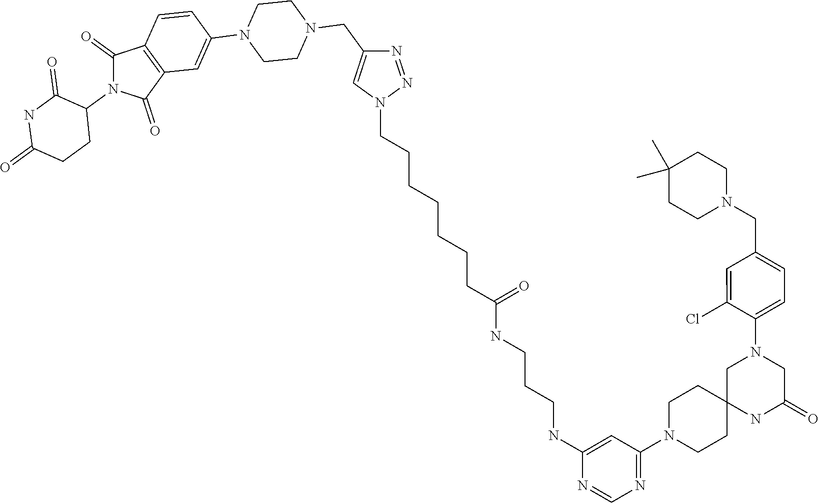 [SMILES: CC1(C)CCN(Cc2ccc(N3CC(=O)NC4(CCN(c5cc(NCCCNC(=O)CCCCCCCn6cc(CN7CCN(c8ccc9c(c8)C(=O)N(C8CCC(=O)NC8=O)C9=O)CC7)nn6)ncn5)CC4)C3)c(Cl)c2)CC1] |
| 233 | 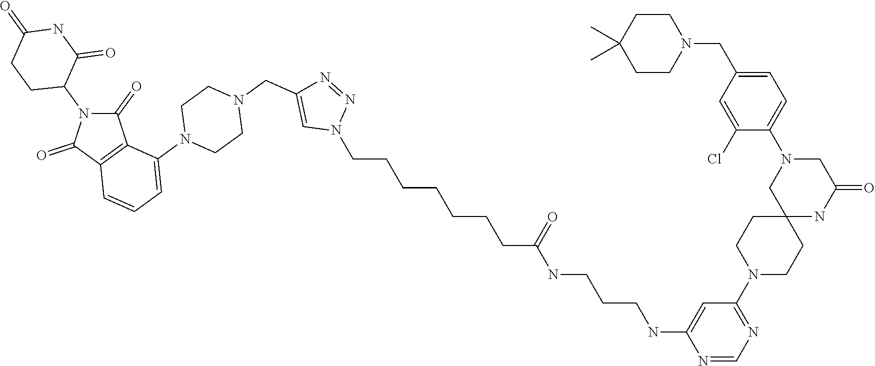 [SMILES: CC1(C)CCN(Cc2ccc(N3CC(=O)NC4(CCN(c5cc(NCCCNC(=O)CCCCCCCn6cc(CN7CCN(c8cccc9c8C(=O)N(C8CCC(=O)NC8=O)C9=O)CC7)nn6)ncn5)CC4)C3)c(Cl)c2)CC1] |
| 234 | 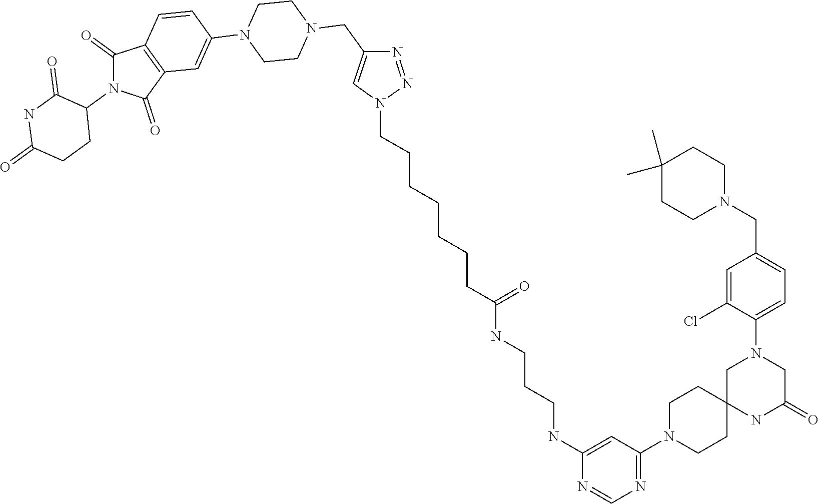 [SMILES: CC1(C)CCN(Cc2ccc(N3CC(=O)NC4(CCN(c5cc(NCCCNC(=O)CCCCCCCn6cc(CN7CCN(c8ccc9c(c8)C(=O)N(C8CCC(=O)NC8=O)C9=O)CC7)nn6)ncn5)CC4)C3)c(Cl)c2)CC1] |
| 235 | 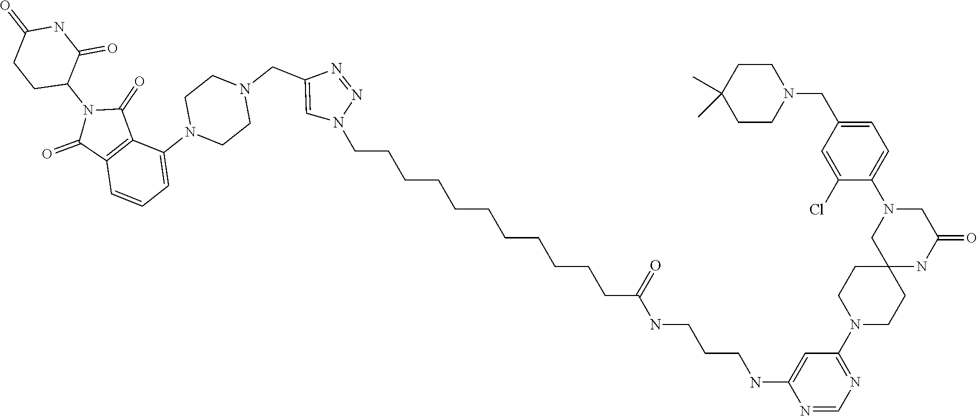 [SMILES: CC1(C)CCN(Cc2ccc(N3CC(=O)NC4(CCN(c5cc(NCCCNC(=O)CCCCCCCCCCCn6cc(CN7CCN(c8cccc9c8C(=O)N(C8CCC(=O)NC8=O)C9=O)CC7)nn6)ncn5)CC4)C3)c(Cl)c2)CC1] |
| 236 | 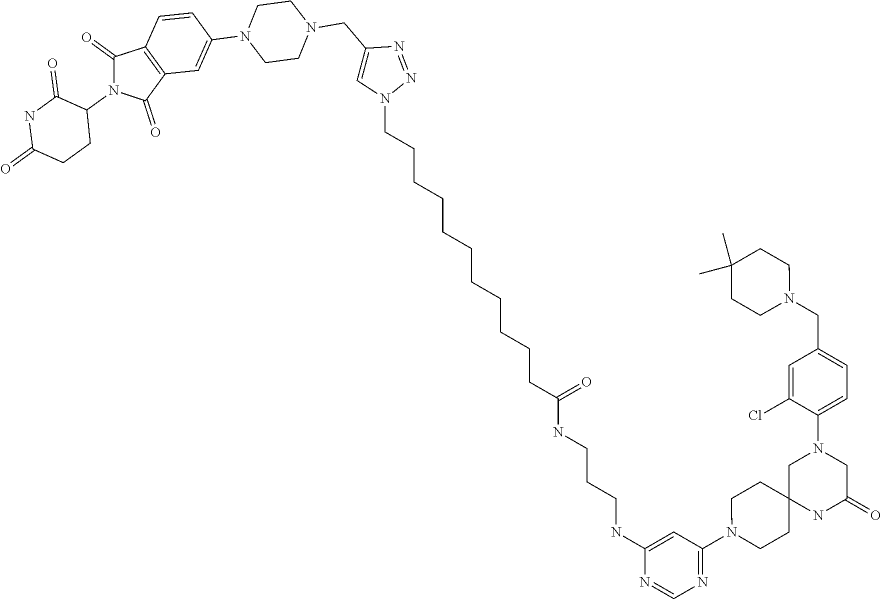 [SMILES: CC1(C)CCN(Cc2ccc(N3CC(=O)NC4(CCN(c5cc(NCCCNC(=O)CCCCCCCCCCCn6cc(CN7CCN(c8ccc9c(c8)C(=O)N(C8CCC(=O)NC8=O)C9=O)CC7)nn6)ncn5)CC4)C3)c(Cl)c2)CC1] |
| 237 | 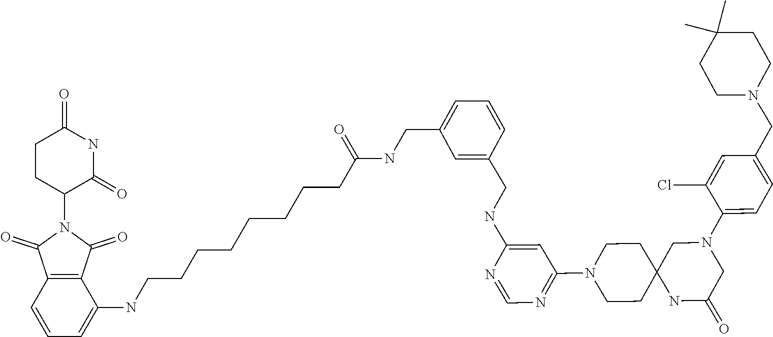 [SMILES: CC1(C)CCN(Cc2ccc(N3CC(=O)NC4(CCN(c5cc(NCc6cccc(CNC(=O)CCCCCCCCNc7cccc8c7C(=O)N(C7CCC(=O)NC7=O)C8=O)c6)ncn5)CC4)C3)c(Cl)c2)CC1] |
| 238 | 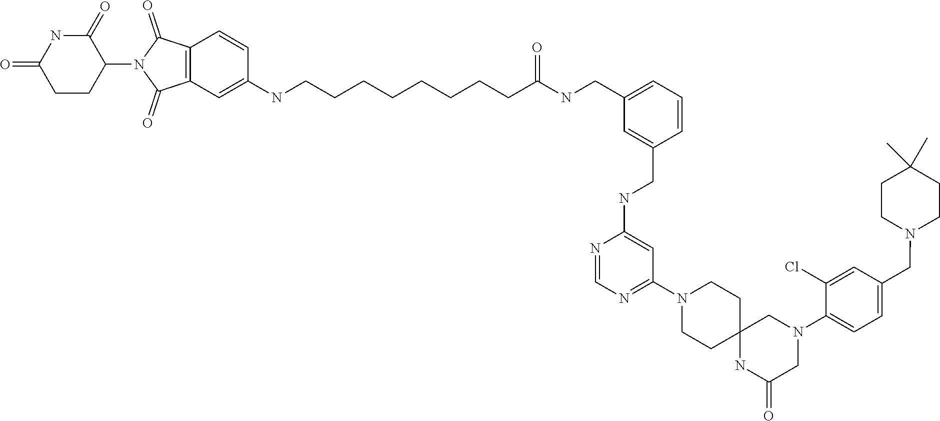 [SMILES: CC1(C)CCN(Cc2ccc(N3CC(=O)NC4(CCN(c5cc(NCc6cccc(CNC(=O)CCCCCCCCNc7ccc8c(c7)C(=O)N(C7CCC(=O)NC7=O)C8=O)c6)ncn5)CC4)C3)c(Cl)c2)CC1] |
| 239 | 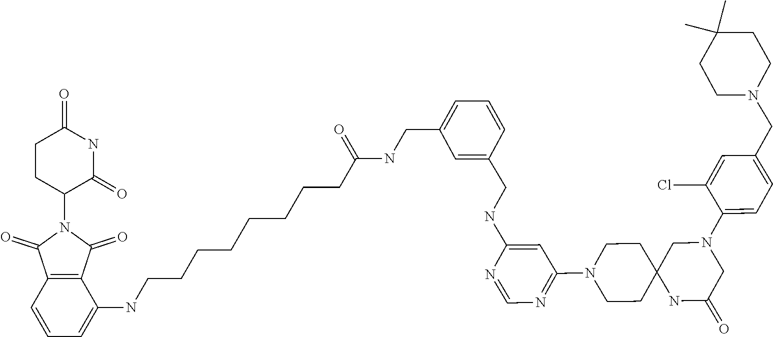 [SMILES: CC1(C)CCN(Cc2ccc(N3CC(=O)NC4(CCN(c5cc(NCc6cccc(CNC(=O)CCCCCCCCNc7cccc8c7C(=O)N(C7CCC(=O)NC7=O)C8=O)c6)ncn5)CC4)C3)c(Cl)c2)CC1] |
| 240 | 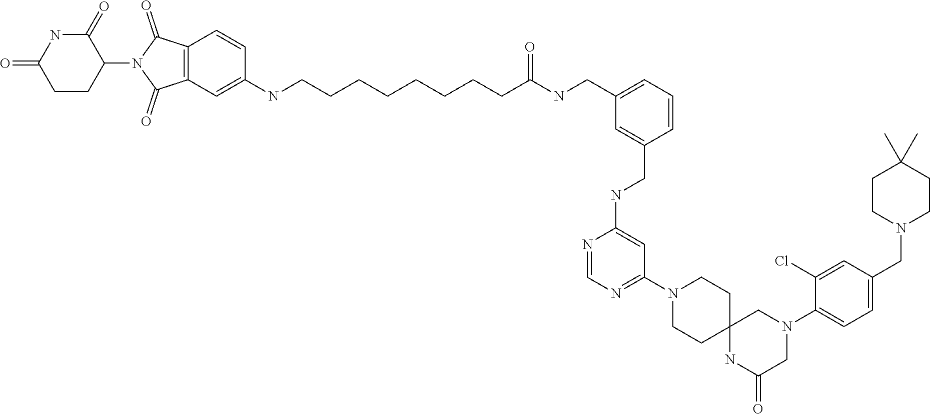 [SMILES: CC1(C)CCN(Cc2ccc(N3CC(=O)NC4(CCN(c5cc(NCc6cccc(CNC(=O)CCCCCCCCNc7ccc8c(c7)C(=O)N(C7CCC(=O)NC7=O)C8=O)c6)ncn5)CC4)C3)c(Cl)c2)CC1] |
| 241 | 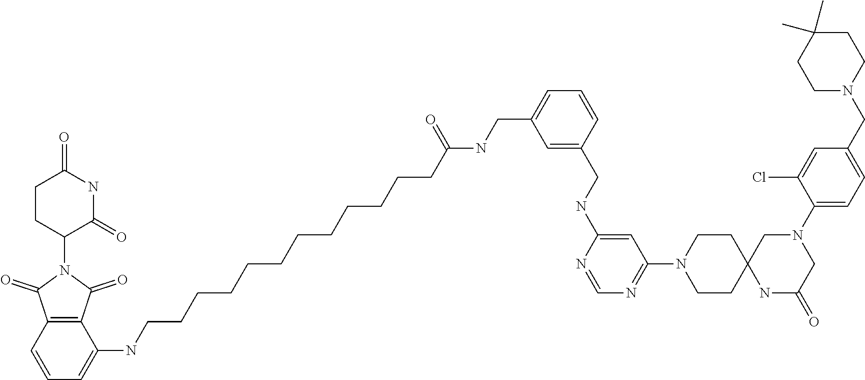 [SMILES: CC1(C)CCN(Cc2ccc(N3CC(=O)NC4(CCN(c5cc(NCc6cccc(CNC(=O)CCCCCCCCCCCCNc7cccc8c7C(=O)N(C7CCC(=O)NC7=O)C8=O)c6)ncn5)CC4)C3)c(Cl)c2)CC1] |
| 242 | 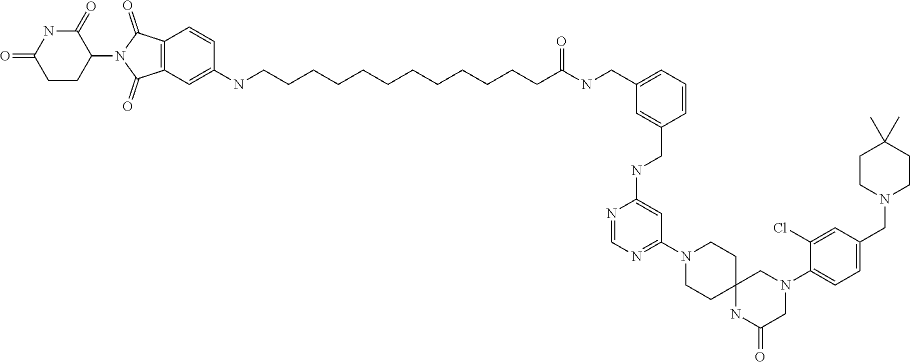 [SMILES: CC1(C)CCN(Cc2ccc(N3CC(=O)NC4(CCN(c5cc(NCc6cccc(CNC(=O)CCCCCCCCCCCCNc7ccc8c(c7)C(=O)N(C7CCC(=O)NC7=O)C8=O)c6)ncn5)CC4)C3)c(Cl)c2)CC1] |
| 243 | 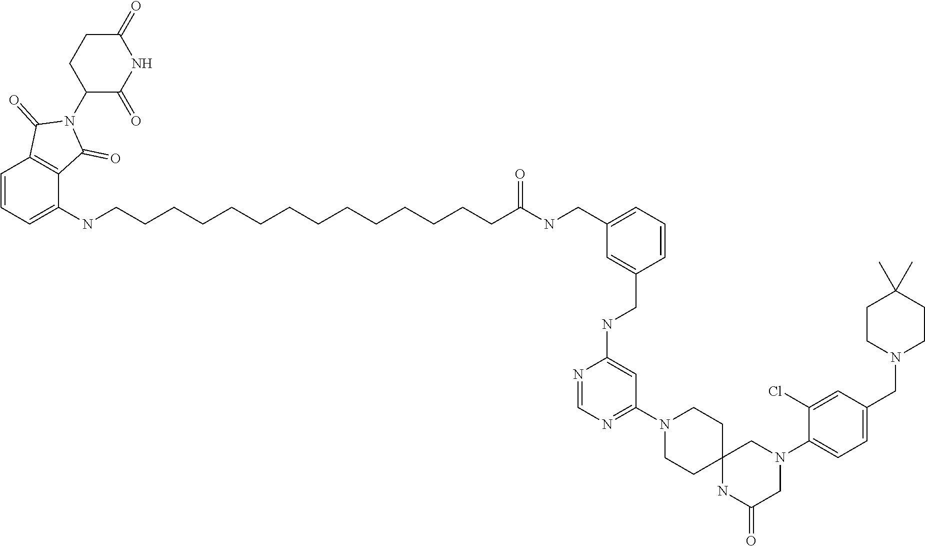 [SMILES: CC1(C)CCN(Cc2ccc(N3CC(=O)NC4(CCN(c5cc(NCc6cccc(CNC(=O)CCCCCCCCCCCCCCNc7cccc8c7C(=O)N(C7CCC(=O)NC7=O)C8=O)c6)ncn5)CC4)C3)c(Cl)c2)CC1] |
| 244 | 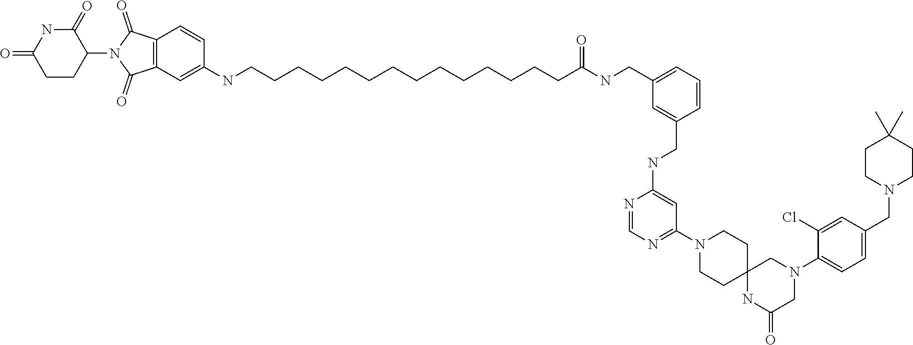 [SMILES: CC1(C)CCN(Cc2ccc(N3CC(=O)NC4(CCN(c5cc(NCc6cccc(CNC(=O)CCCCCCCCCCCCCCNc7ccc8c(c7)C(=O)N(C7CCC(=O)NC7=O)C8=O)c6)ncn5)CC4)C3)c(Cl)c2)CC1] |
| 245 | 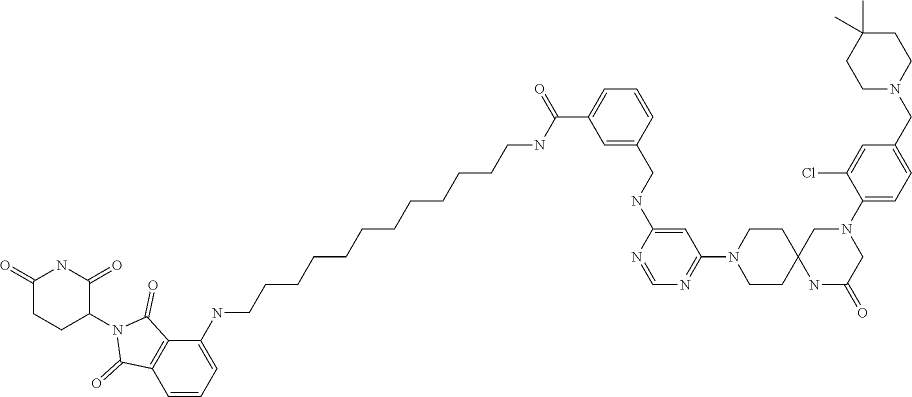 [SMILES: CC1(C)CCN(Cc2ccc(N3CC(=O)NC4(CCN(c5cc(NCc6cccc(C(=O)NCCCCCCCCCCCCNc7cccc8c7C(=O)N(C7CCC(=O)NC7=O)C8=O)c6)ncn5)CC4)C3)c(Cl)c2)CC1] |
| 246 | 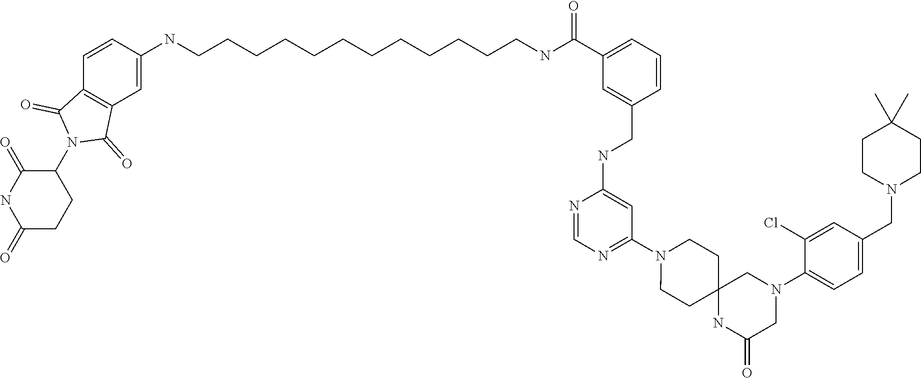 [SMILES: CC1(C)CCN(Cc2ccc(N3CC(=O)NC4(CCN(c5cc(NCc6cccc(C(=O)NCCCCCCCCCCCCNc7ccc8c(c7)C(=O)N(C7CCC(=O)NC7=O)C8=O)c6)ncn5)CC4)C3)c(Cl)c2)CC1] |
| 247 | 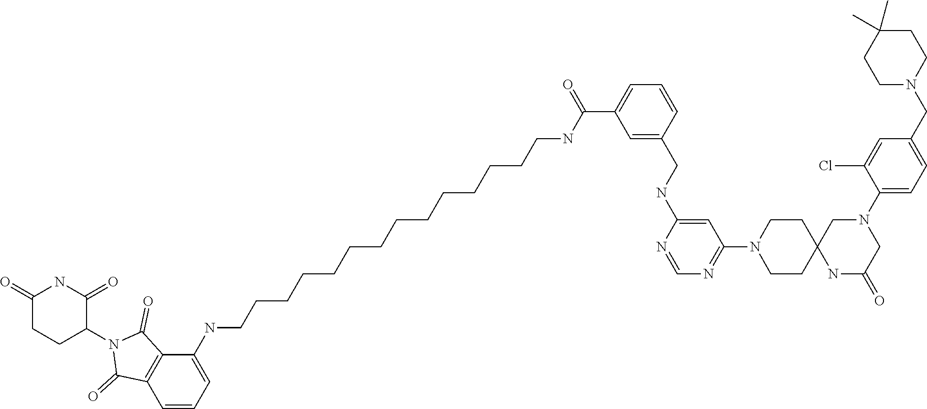 [SMILES: CC1(C)CCN(Cc2ccc(N3CC(=O)NC4(CCN(c5cc(NCc6cccc(C(=O)NCCCCCCCCCCCCCCNc7cccc8c7C(=O)N(C7CCC(=O)NC7=O)C8=O)c6)ncn5)CC4)C3)c(Cl)c2)CC1] |
| 248 | 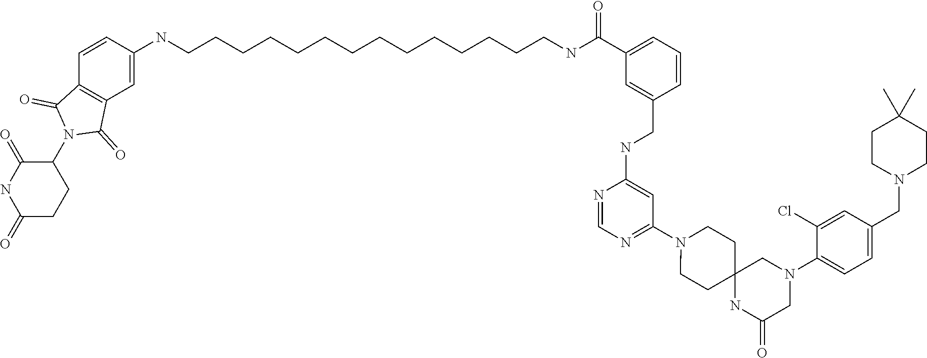 [SMILES: CC1(C)CCN(Cc2ccc(N3CC(=O)NC4(CCN(c5cc(NCc6cccc(C(=O)NCCCCCCCCCCCCCCNc7ccc8c(c7)C(=O)N(C7CCC(=O)NC7=O)C8=O)c6)ncn5)CC4)C3)c(Cl)c2)CC1] |
| 249 | 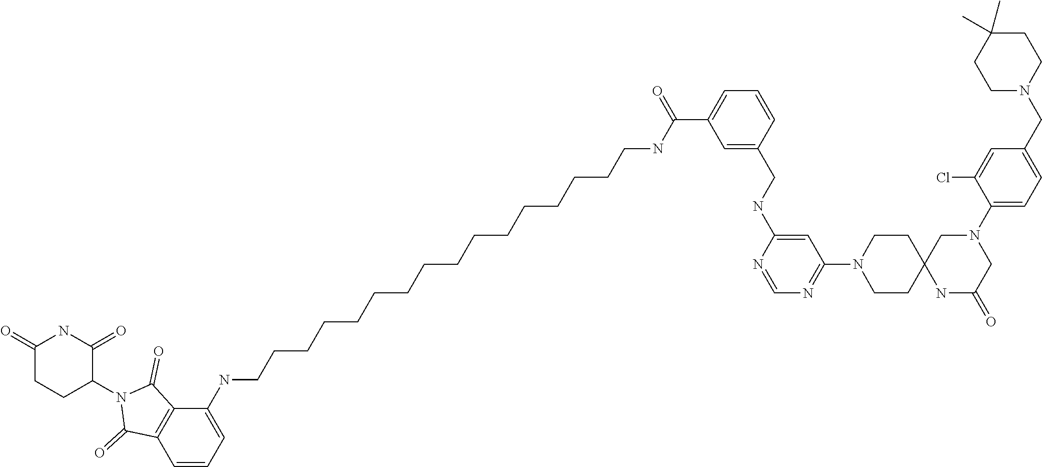 [SMILES: CC1(C)CCN(Cc2ccc(N3CC(=O)NC4(CCN(c5cc(NCc6cccc(C(=O)NCCCCCCCCCCCCCCCCNc7cccc8c7C(=O)N(C7CCC(=O)NC7=O)C8=O)c6)ncn5)CC4)C3)c(Cl)c2)CC1] |
| 250 | 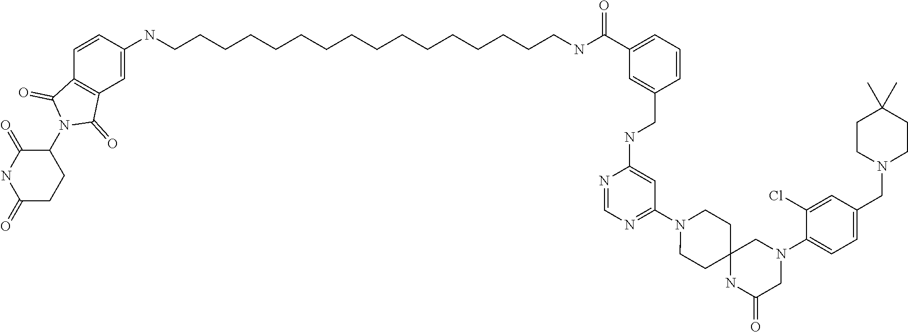 [SMILES: CC1(C)CCN(Cc2ccc(N3CC(=O)NC4(CCN(c5cc(NCc6cccc(C(=O)NCCCCCCCCCCCCCCCCNc7ccc8c(c7)C(=O)N(C7CCC(=O)NC7=O)C8=O)c6)ncn5)CC4)C3)c(Cl)c2)CC1] |
| 251 | 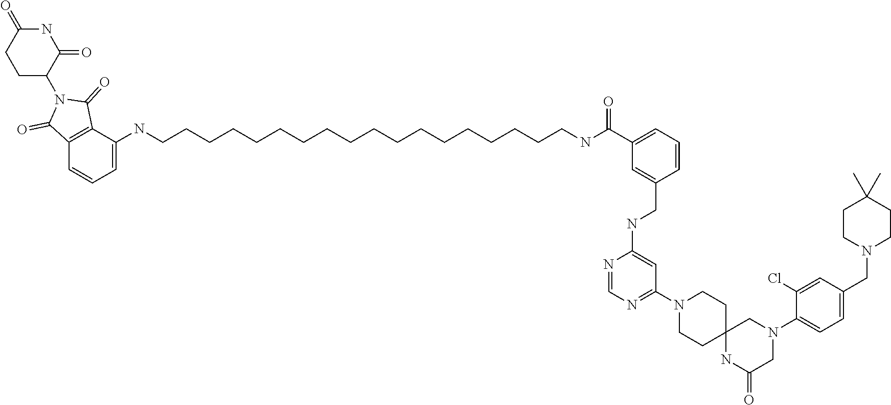 [SMILES: CC1(C)CCN(Cc2ccc(N3CC(=O)NC4(CCN(c5cc(NCc6cccc(C(=O)NCCCCCCCCCCCCCCCCCCNc7cccc8c7C(=O)N(C7CCC(=O)NC7=O)C8=O)c6)ncn5)CC4)C3)c(Cl)c2)CC1] |
| 252 | 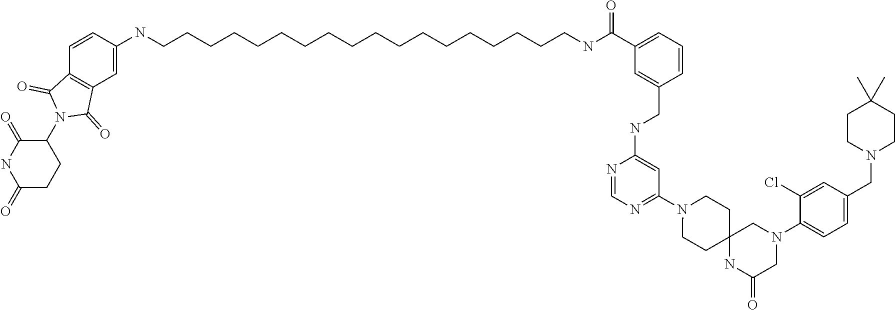 [SMILES: CC1(C)CCN(Cc2ccc(N3CC(=O)NC4(CCN(c5cc(NCc6cccc(C(=O)NCCCCCCCCCCCCCCCCCCNc7ccc8c(c7)C(=O)N(C7CCC(=O)NC7=O)C8=O)c6)ncn5)CC4)C3)c(Cl)c2)CC1] |
| 253 | 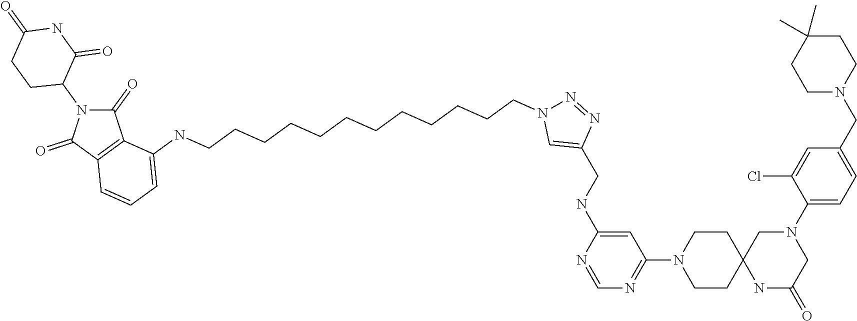 [SMILES: CC1(C)CCN(Cc2ccc(N3CC(=O)NC4(CCN(c5cc(NCc6cn(CCCCCCCCCCCCNc7cccc8c7C(=O)N(C7CCC(=O)NC7=O)C8=O)nn6)ncn5)CC4)C3)c(Cl)c2)CC1] |
| 254 | 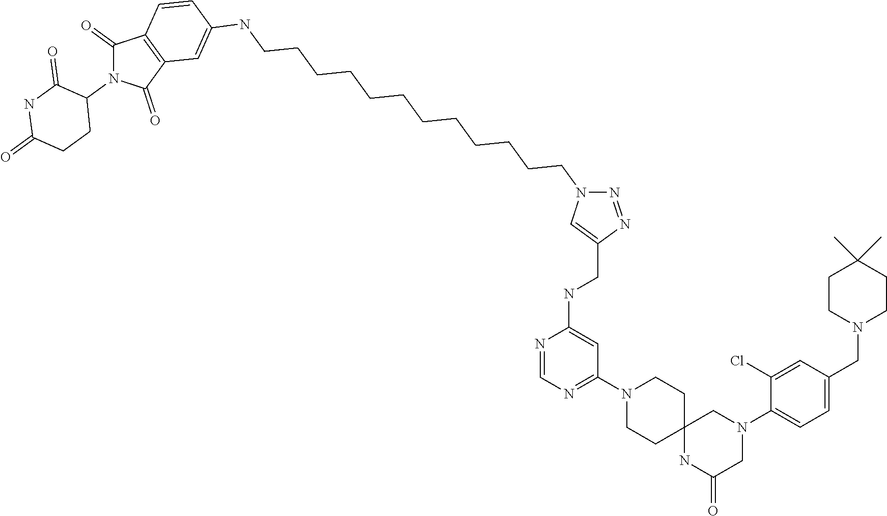 [SMILES: CC1(C)CCN(Cc2ccc(N3CC(=O)NC4(CCN(c5cc(NCc6cn(CCCCCCCCCCCCNc7ccc8c(c7)C(=O)N(C7CCC(=O)NC7=O)C8=O)nn6)ncn5)CC4)C3)c(Cl)c2)CC1] |
| 255 | 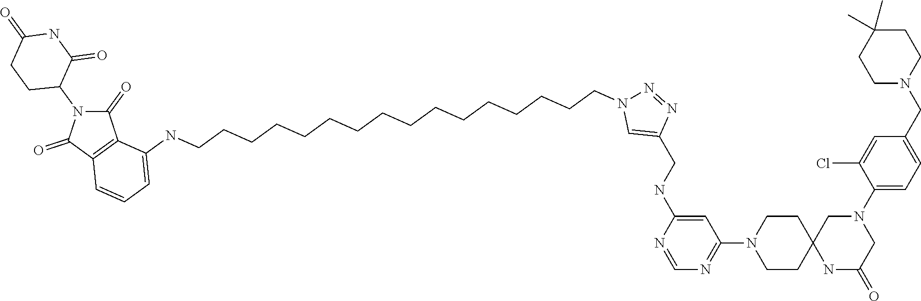 [SMILES: CC1(C)CCN(Cc2ccc(N3CC(=O)NC4(CCN(c5cc(NCc6cn(CCCCCCCCCCCCCCCCNc7cccc8c7C(=O)N(C7CCC(=O)NC7=O)C8=O)nn6)ncn5)CC4)C3)c(Cl)c2)CC1] |
| 256 | 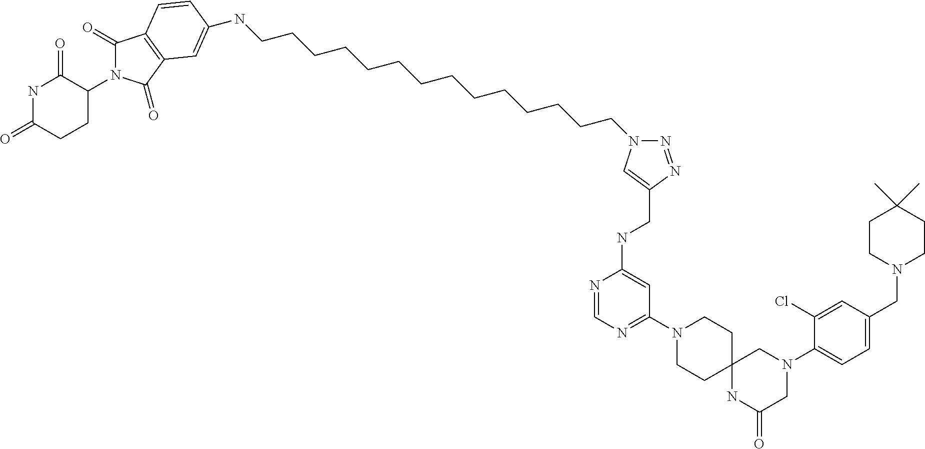 [SMILES: CC1(C)CCN(Cc2ccc(N3CC(=O)NC4(CCN(c5cc(NCc6cn(CCCCCCCCCCCCCCNc7ccc8c(c7)C(=O)N(C7CCC(=O)NC7=O)C8=O)nn6)ncn5)CC4)C3)c(Cl)c2)CC1] |
| 257 | 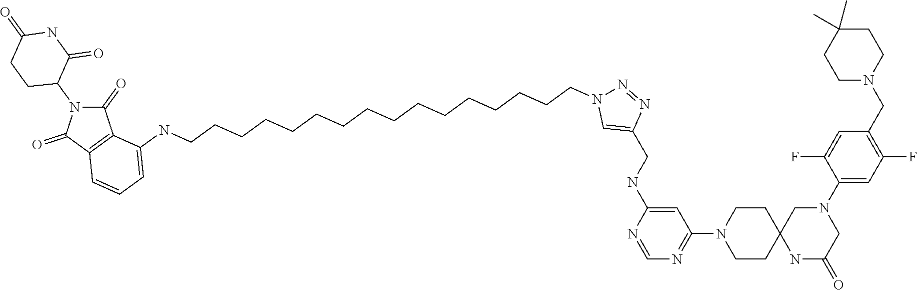 [SMILES: CC1(C)CCN(Cc2cc(F)c(N3CC(=O)NC4(CCN(c5cc(NCc6cn(CCCCCCCCCCCCCCCCNc7cccc8c7C(=O)N(C7CCC(=O)NC7=O)C8=O)nn6)ncn5)CC4)C3)cc2F)CC1] |
| 258 | 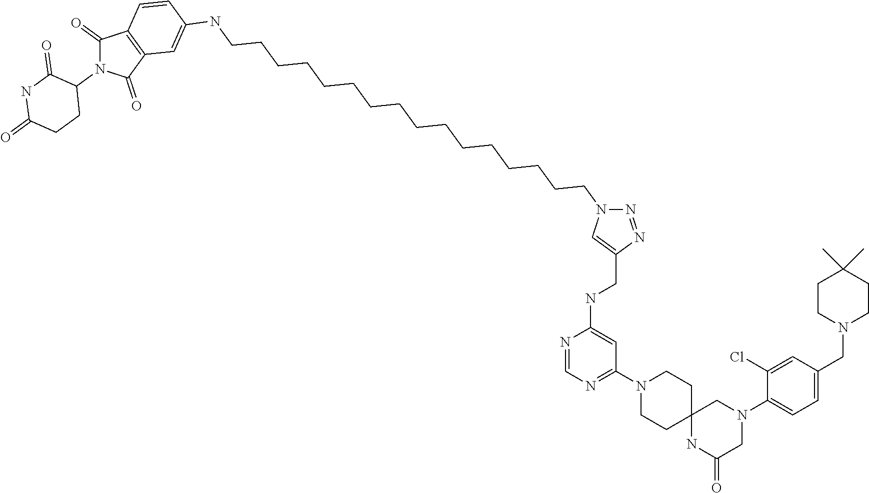 [SMILES: CC1(C)CCN(Cc2ccc(N3CC(=O)NC4(CCN(c5cc(NCc6cn(CCCCCCCCCCCCCCCCNc7ccc8c(c7)C(=O)N(C7CCC(=O)NC7=O)C8=O)nn6)ncn5)CC4)C3)c(Cl)c2)CC1] |
| 259 | 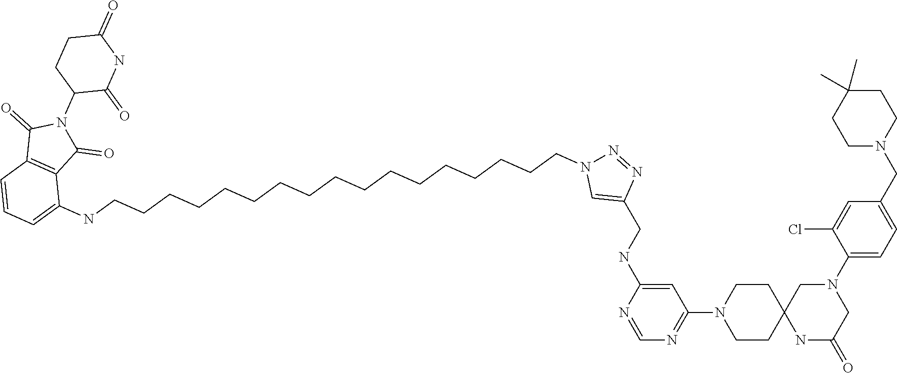 [SMILES: CC1(C)CCN(Cc2ccc(N3CC(=O)NC4(CCN(c5cc(NCc6cn(CCCCCCCCCCCCCCCCCNc7cccc8c7C(=O)N(C7CCC(=O)NC7=O)C8=O)nn6)ncn5)CC4)C3)c(Cl)c2)CC1] |
| 260 | 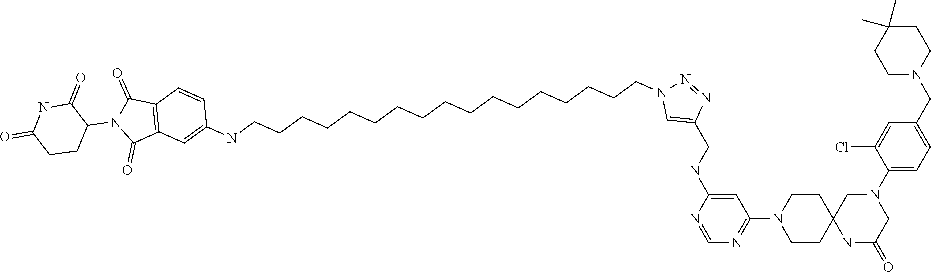 [SMILES: CC1(C)CCN(Cc2ccc(N3CC(=O)NC4(CCN(c5cc(NCc6cn(CCCCCCCCCCCCCCCCCNc7ccc8c(c7)C(=O)N(C7CCC(=O)NC7=O)C8=O)nn6)ncn5)CC4)C3)c(Cl)c2)CC1] |
| 261 | 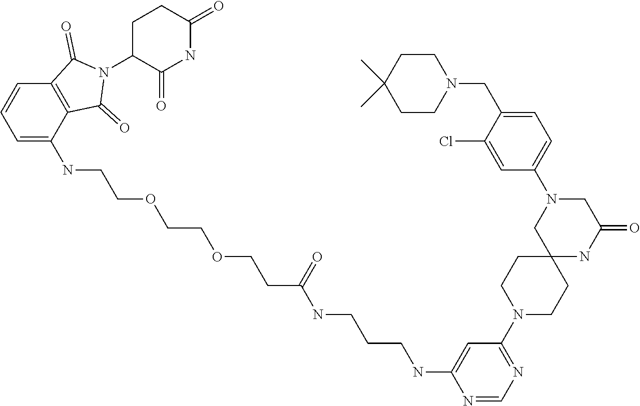 [SMILES: CC1(C)CCN(Cc2ccc(N3CC(=O)NC4(CCN(c5cc(NCCCNC(=O)CCOCCOCCNc6cccc7c6C(=O)N(C6CCC(=O)NC6=O)C7=O)ncn5)CC4)C3)cc2Cl)CC1] |
| 262 | 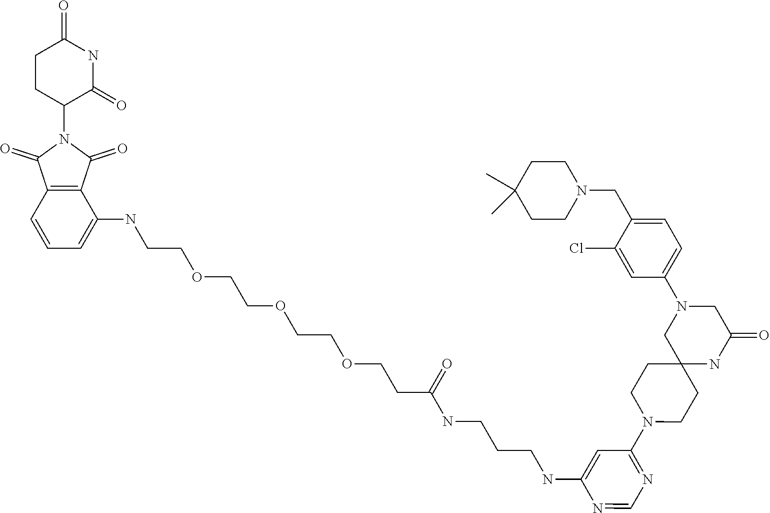 [SMILES: CC1(C)CCN(Cc2ccc(N3CC(=O)NC4(CCN(c5cc(NCCCNC(=O)CCOCCOCCOCCNc6cccc7c6C(=O)N(C6CCC(=O)NC6=O)C7=O)ncn5)CC4)C3)cc2Cl)CC1] |
| 263 | 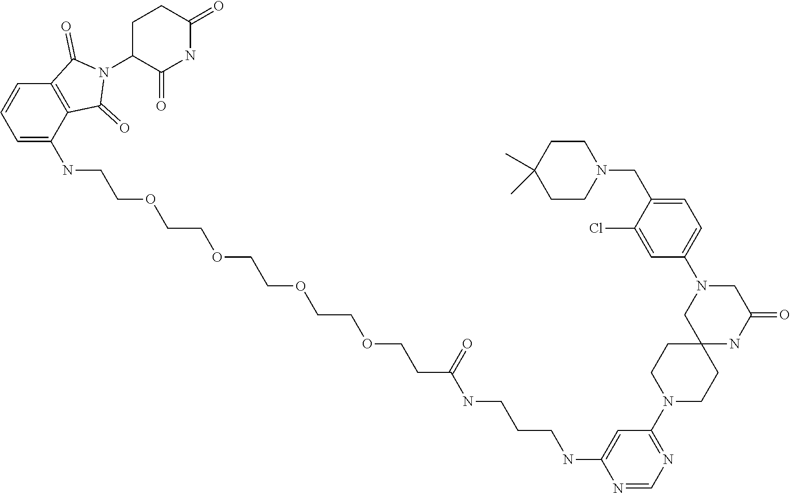 [SMILES: CC1(C)CCN(Cc2ccc(N3CC(=O)NC4(CCN(c5cc(NCCCNC(=O)CCOCCOCCOCCOCCNc6cccc7c6C(=O)N(C6CCC(=O)NC6=O)C7=O)ncn5)CC4)C3)cc2Cl)CC1] |
| 264 | 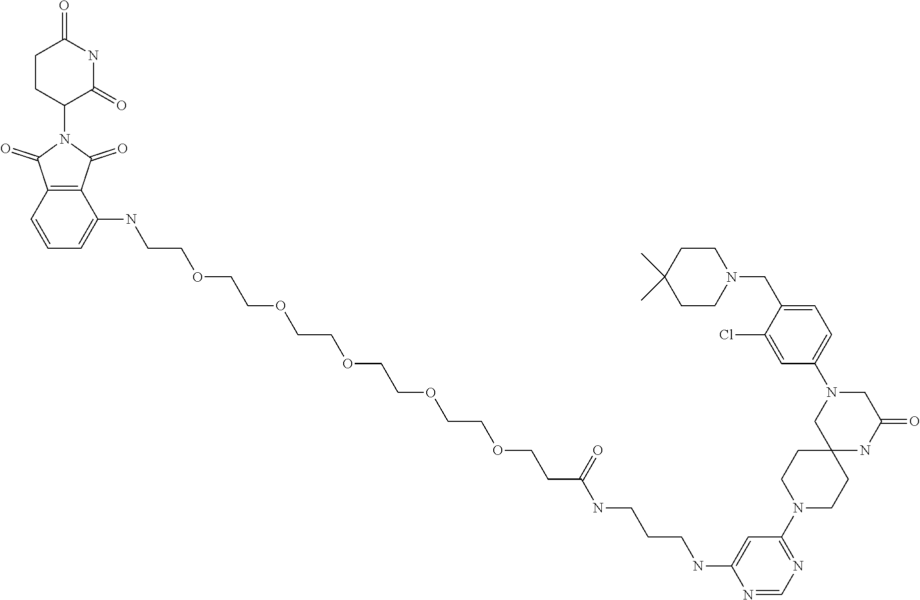 [SMILES: CC1(C)CCN(Cc2ccc(N3CC(=O)NC4(CCN(c5cc(NCCCNC(=O)CCOCCOCCOCCOCCOCCNc6cccc7c6C(=O)N(C6CCC(=O)NC6=O)C7=O)ncn5)CC4)C3)cc2Cl)CC1] |
| 265 | 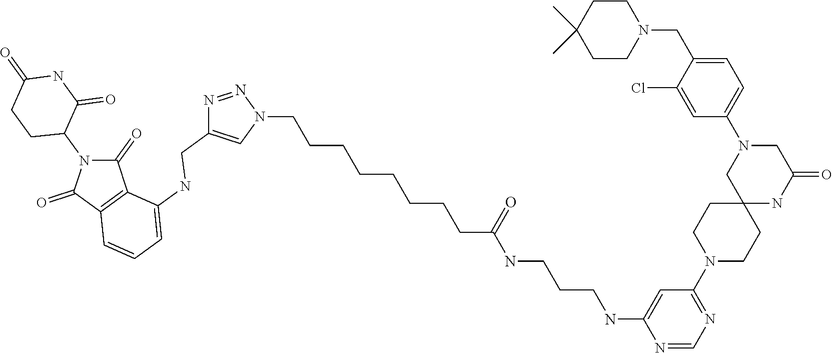 [SMILES: CC1(C)CCN(Cc2ccc(N3CC(=O)NC4(CCN(c5cc(NCCCNC(=O)CCCCCCCCn6cc(CNc7cccc8c7C(=O)N(C7CCC(=O)NC7=O)C8=O)nn6)ncn5)CC4)C3)cc2Cl)CC1] |
| 266 | 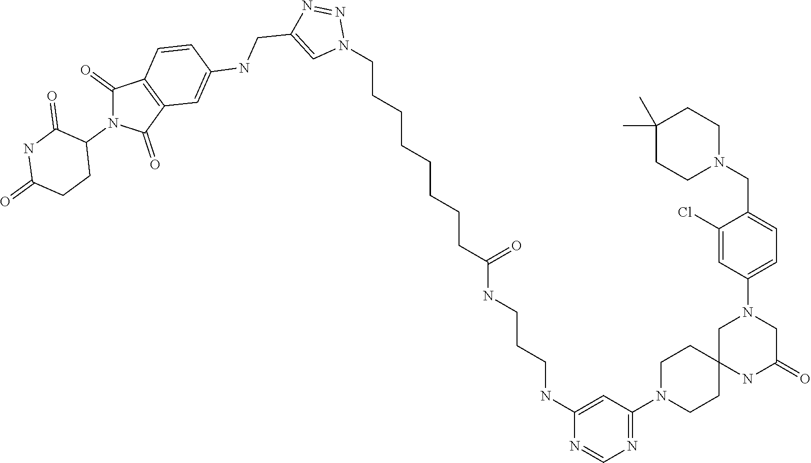 [SMILES: CC1(C)CCN(Cc2ccc(N3CC(=O)NC4(CCN(c5cc(NCCCNC(=O)CCCCCCCCn6cc(CNc7ccc8c(c7)C(=O)N(C7CCC(=O)NC7=O)C8=O)nn6)ncn5)CC4)C3)cc2Cl)CC1] |
| 267 | 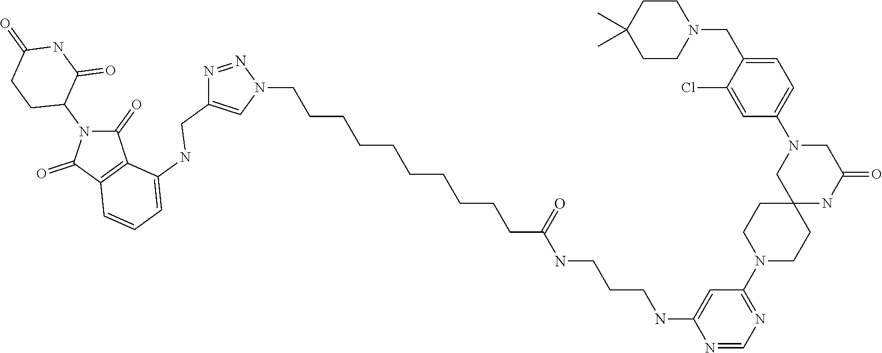 [SMILES: CC1(C)CCN(Cc2ccc(N3CC(=O)NC4(CCN(c5cc(NCCCNC(=O)CCCCCCCCCCn6cc(CNc7cccc8c7C(=O)N(C7CCC(=O)NC7=O)C8=O)nn6)ncn5)CC4)C3)cc2Cl)CC1] |
| 268 | 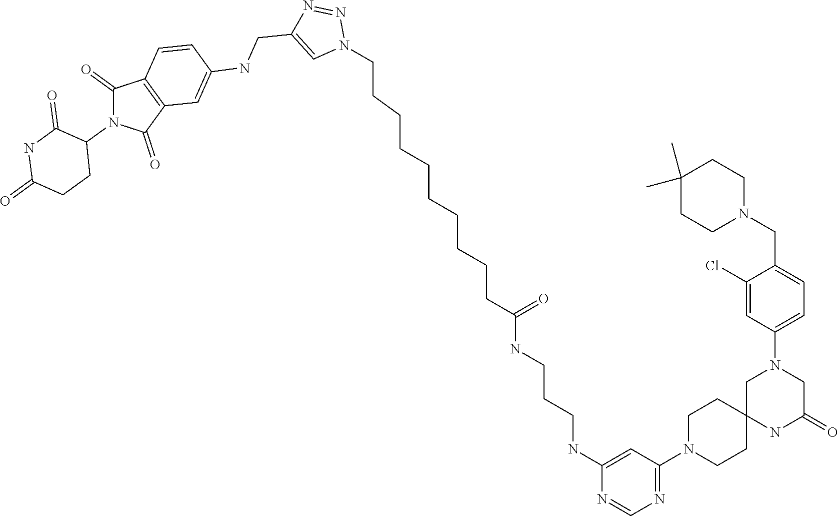 [SMILES: CC1(C)CCN(Cc2ccc(N3CC(=O)NC4(CCN(c5cc(NCCCNC(=O)CCCCCCCCCCn6cc(CNc7ccc8c(c7)C(=O)N(C7CCC(=O)NC7=O)C8=O)nn6)ncn5)CC4)C3)cc2Cl)CC1] |
| 269 | 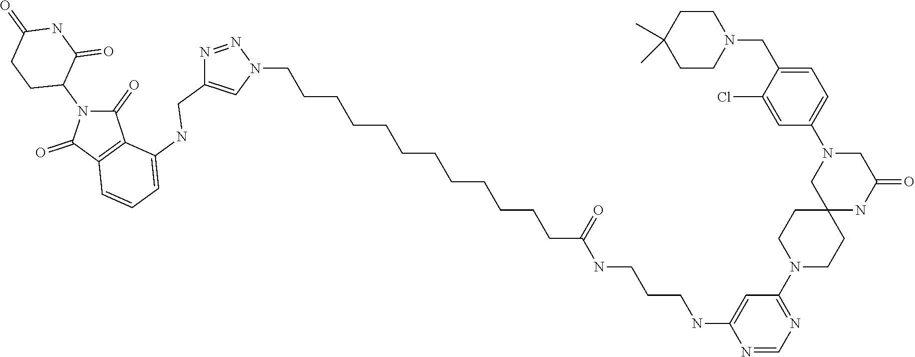 [SMILES: CC1(C)CCN(Cc2ccc(N3CC(=O)NC4(CCN(c5cc(NCCCNC(=O)CCCCCCCCCCCCn6cc(CNc7cccc8c7C(=O)N(C7CCC(=O)NC7=O)C8=O)nn6)ncn5)CC4)C3)cc2Cl)CC1] |
| 270 | 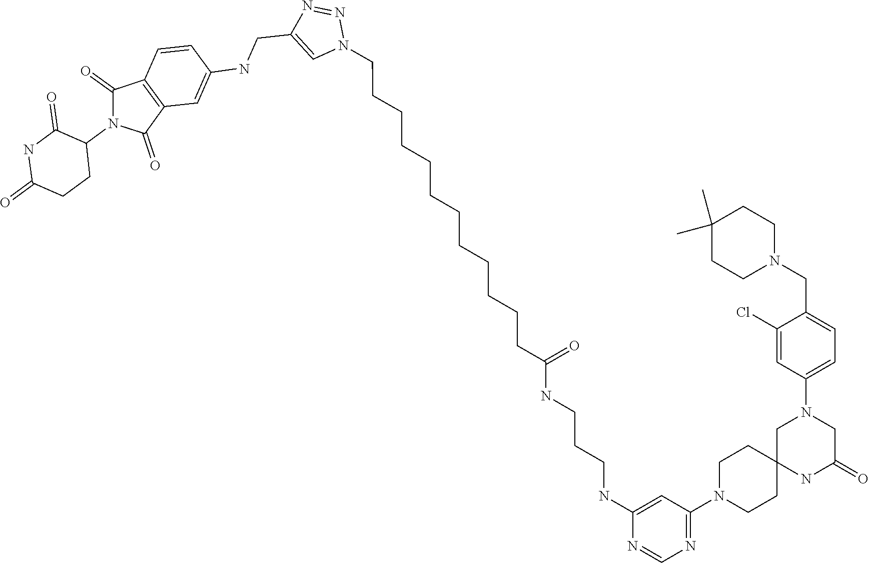 [SMILES: CC1(C)CCN(Cc2ccc(N3CC(=O)NC4(CCN(c5cc(NCCCNC(=O)CCCCCCCCCCCCn6cc(CNc7ccc8c(c7)C(=O)N(C7CCC(=O)NC7=O)C8=O)nn6)ncn5)CC4)C3)cc2Cl)CC1] |
| 271 | 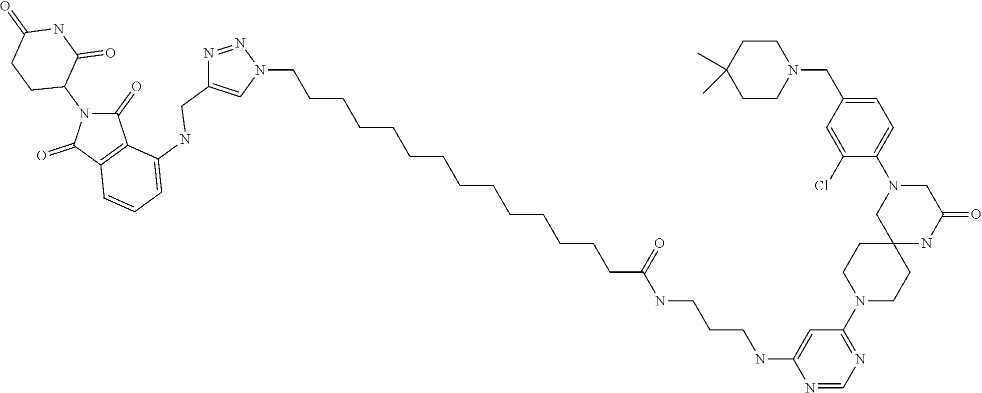 [SMILES: CC1(C)CCN(Cc2ccc(N3CC(=O)NC4(CCN(c5cc(NCCCNC(=O)CCCCCCCCCCCCCCn6cc(CNc7cccc8c7C(=O)N(C7CCC(=O)NC7=O)C8=O)nn6)ncn5)CC4)C3)c(Cl)c2)CC1] |
| 272 | 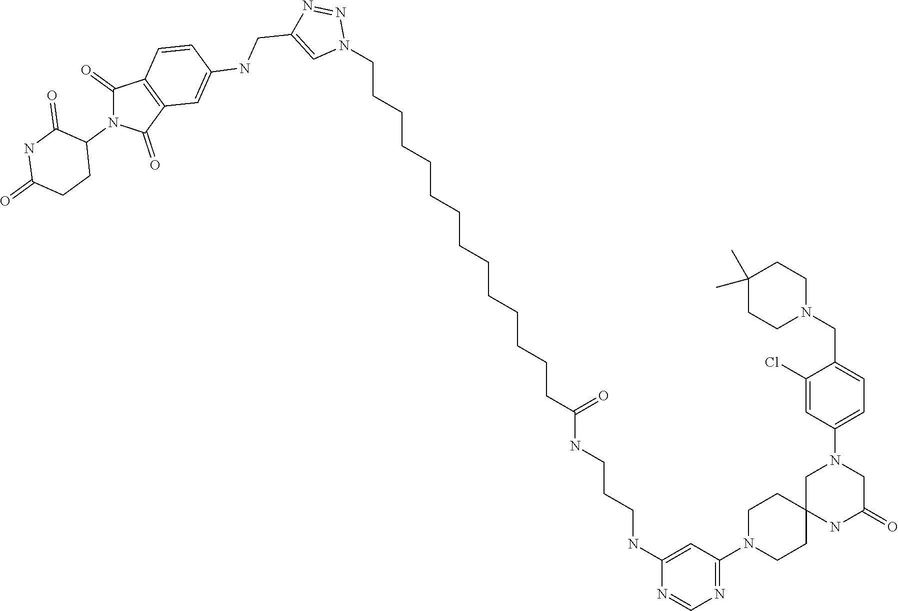 [SMILES: CC1(C)CCN(Cc2ccc(N3CC(=O)NC4(CCN(c5cc(NCCCNC(=O)CCCCCCCCCCCCCCn6cc(CNc7ccc8c(c7)C(=O)N(C7CCC(=O)NC7=O)C8=O)nn6)ncn5)CC4)C3)cc2Cl)CC1] |
| 273 | 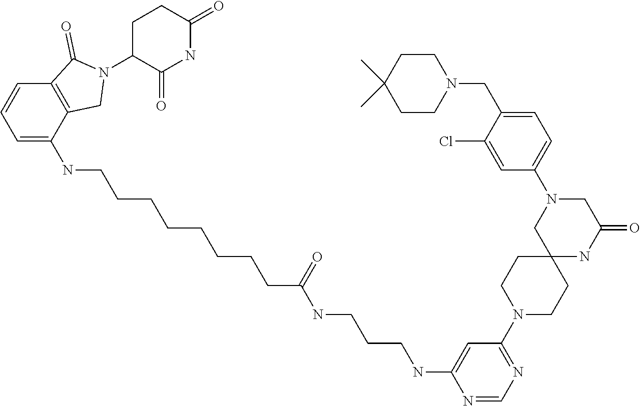 [SMILES: CC1(C)CCN(Cc2ccc(N3CC(=O)NC4(CCN(c5cc(NCCCNC(=O)CCCCCCCCNc6cccc7c6CN(C6CCC(=O)NC6=O)C7=O)ncn5)CC4)C3)cc2Cl)CC1] |
| 274 | 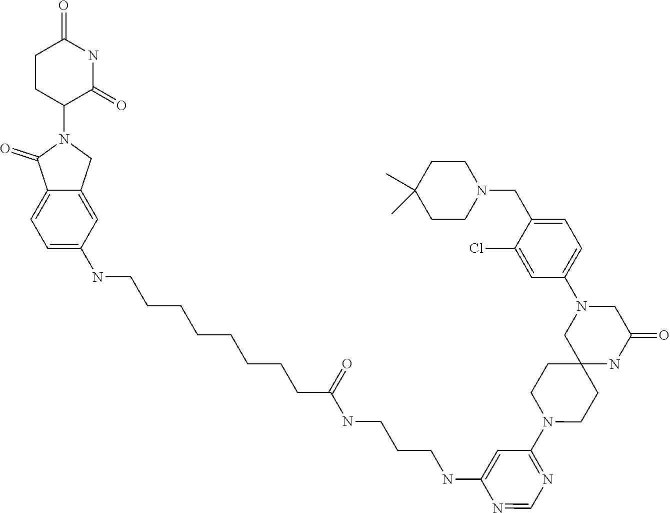 [SMILES: CC1(C)CCN(Cc2ccc(N3CC(=O)NC4(CCN(c5cc(NCCCNC(=O)CCCCCCCCNc6ccc7c(c6)CN(C6CCC(=O)NC6=O)C7=O)ncn5)CC4)C3)cc2Cl)CC1] |
| 275 | 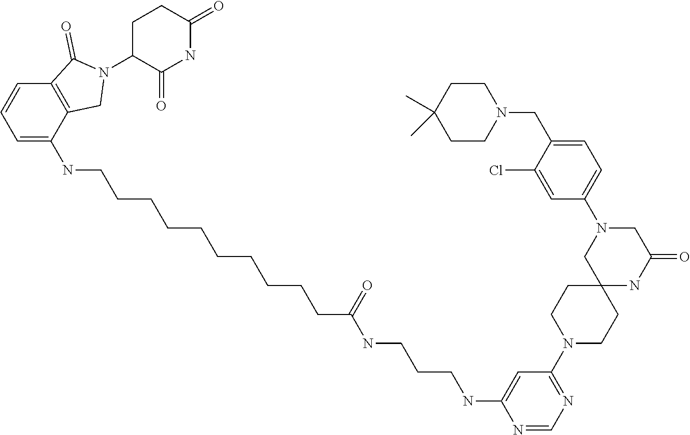 [SMILES: CC1(C)CCN(Cc2ccc(N3CC(=O)NC4(CCN(c5cc(NCCCNC(=O)CCCCCCCCCCNc6cccc7c6CN(C6CCC(=O)NC6=O)C7=O)ncn5)CC4)C3)cc2Cl)CC1] |
| 276 | 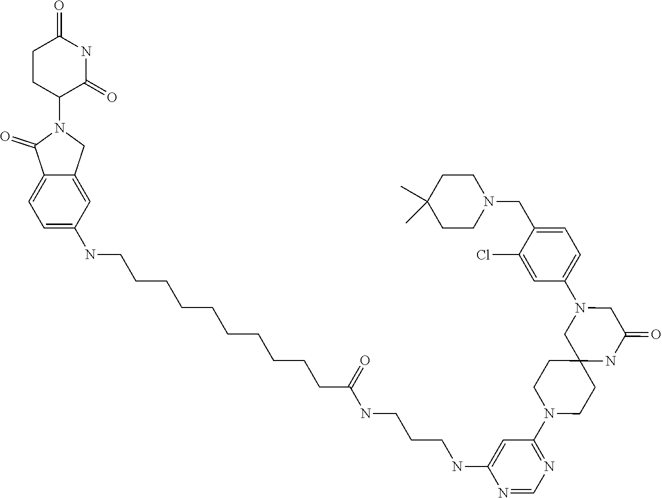 [SMILES: CC1(C)CCN(Cc2ccc(N3CC(=O)NC4(CCN(c5cc(NCCCNC(=O)CCCCCCCCCCNc6ccc7c(c6)CN(C6CCC(=O)NC6=O)C7=O)ncn5)CC4)C3)cc2Cl)CC1] |
| 277 | 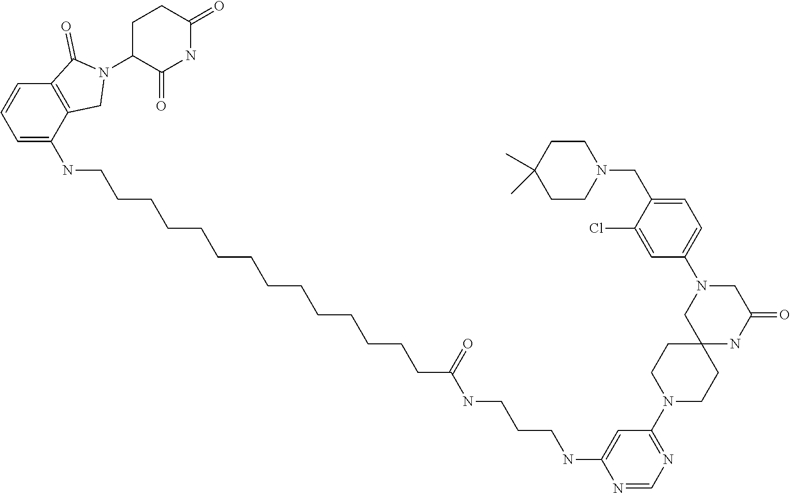 [SMILES: CC1(C)CCN(Cc2ccc(N3CC(=O)NC4(CCN(c5cc(NCCCNC(=O)CCCCCCCCCCCCCCNc6cccc7c6CN(C6CCC(=O)NC6=O)C7=O)ncn5)CC4)C3)cc2Cl)CC1] |
| 278 | 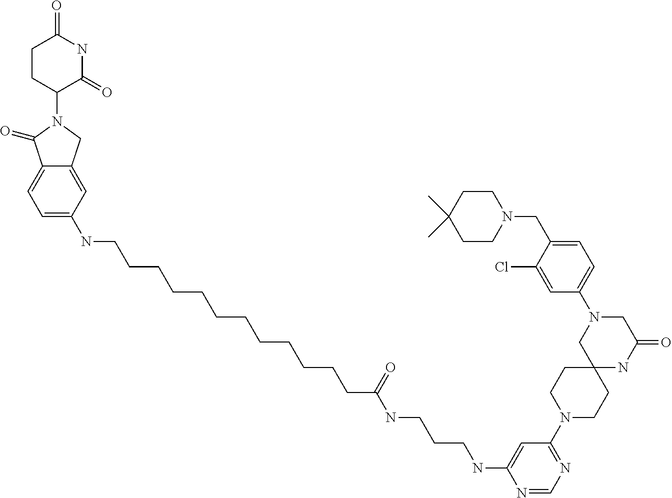 [SMILES: CC1(C)CCN(Cc2ccc(N3CC(=O)NC4(CCN(c5cc(NCCCNC(=O)CCCCCCCCCCCCNc6ccc7c(c6)CN(C6CCC(=O)NC6=O)C7=O)ncn5)CC4)C3)cc2Cl)CC1] |
| 279 | 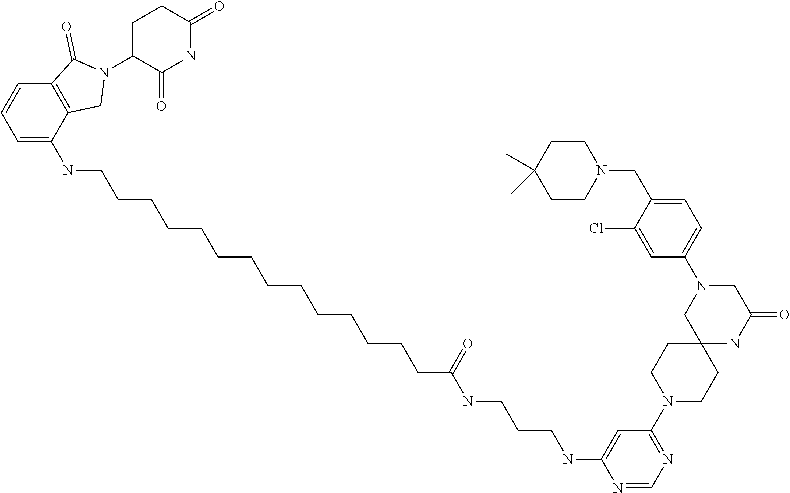 [SMILES: CC1(C)CCN(Cc2ccc(N3CC(=O)NC4(CCN(c5cc(NCCCNC(=O)CCCCCCCCCCCCCCNc6cccc7c6CN(C6CCC(=O)NC6=O)C7=O)ncn5)CC4)C3)cc2Cl)CC1] |
| 280 | 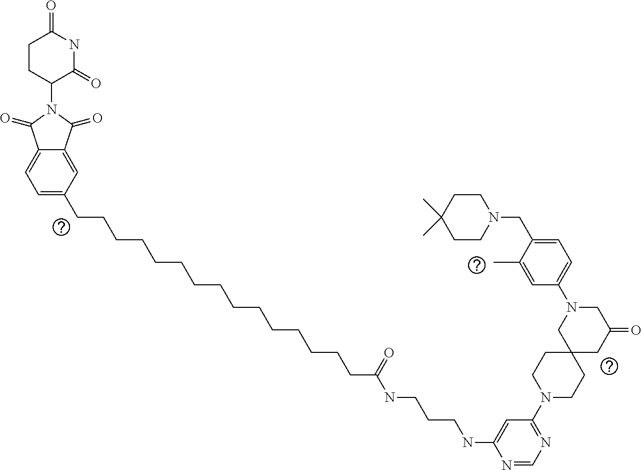 [SMILES: Cc1cc(N2CC(=O)CC3(CCN(c4cc(NCCCNC(=O)CCCCCCCCCCCCCCCc5ccc6c(c5)C(=O)N(C5CCC(=O)NC5=O)C6=O)ncn4)CC3)C2)ccc1CN1CCC(C)(C)CC1] |
| 281 | 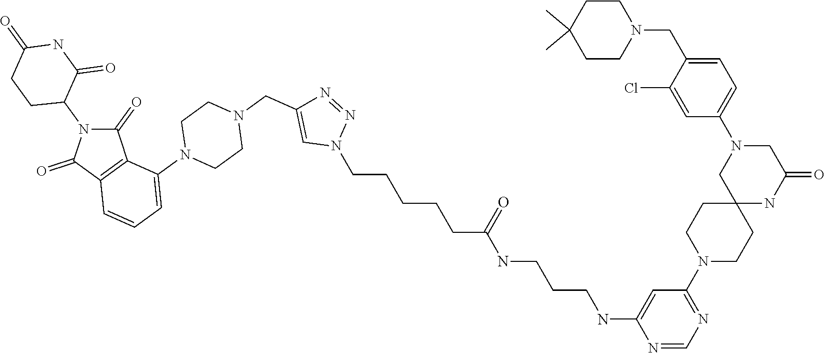 [SMILES: CC1(C)CCN(Cc2ccc(N3CC(=O)NC4(CCN(c5cc(NCCCNC(=O)CCCCCn6cc(CN7CCN(c8cccc9c8C(=O)N(C8CCC(=O)NC8=O)C9=O)CC7)nn6)ncn5)CC4)C3)cc2Cl)CC1] |
| 282 | 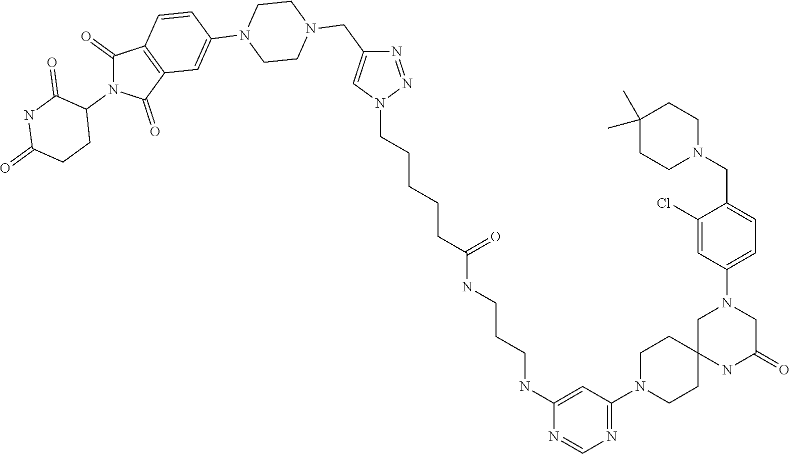 [SMILES: CC1(C)CCN(Cc2ccc(N3CC(=O)NC4(CCN(c5cc(NCCCNC(=O)CCCCCn6cc(CN7CCN(c8ccc9c(c8)C(=O)N(C8CCC(=O)NC8=O)C9=O)CC7)nn6)ncn5)CC4)C3)cc2Cl)CC1] |
| 283 | 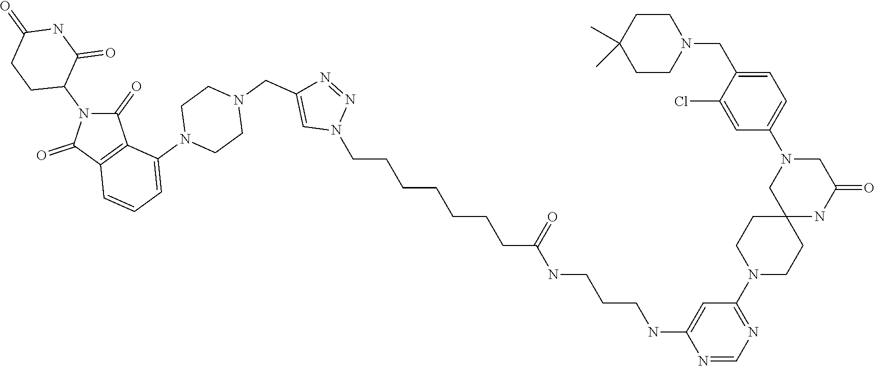 [SMILES: CC1(C)CCN(Cc2ccc(N3CC(=O)NC4(CCN(c5cc(NCCCNC(=O)CCCCCCCn6cc(CN7CCN(c8cccc9c8C(=O)N(C8CCC(=O)NC8=O)C9=O)CC7)nn6)ncn5)CC4)C3)cc2Cl)CC1] |
| 284 | 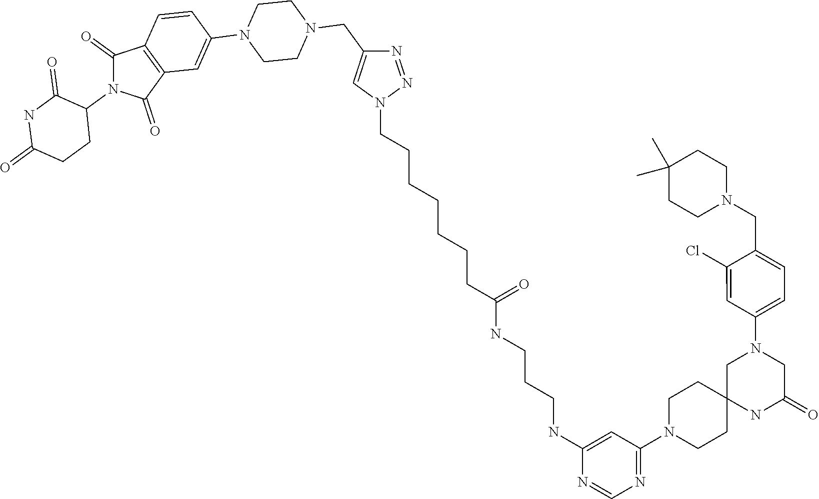 [SMILES: CC1(C)CCN(Cc2ccc(N3CC(=O)NC4(CCN(c5cc(NCCCNC(=O)CCCCCCCn6cc(CN7CCN(c8ccc9c(c8)C(=O)N(C8CCC(=O)NC8=O)C9=O)CC7)nn6)ncn5)CC4)C3)cc2Cl)CC1] |
| 285 | 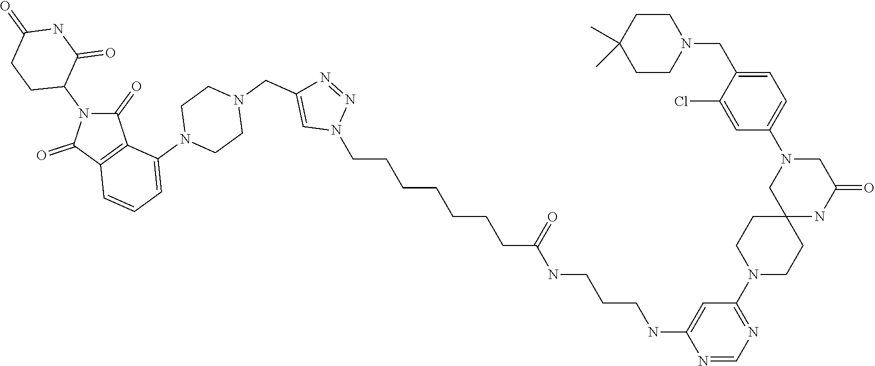 [SMILES: CC1(C)CCN(Cc2ccc(N3CC(=O)NC4(CCN(c5cc(NCCCNC(=O)CCCCCCCn6cc(CN7CCN(c8cccc9c8C(=O)N(C8CCC(=O)NC8=O)C9=O)CC7)nn6)ncn5)CC4)C3)cc2Cl)CC1] |
| 286 | 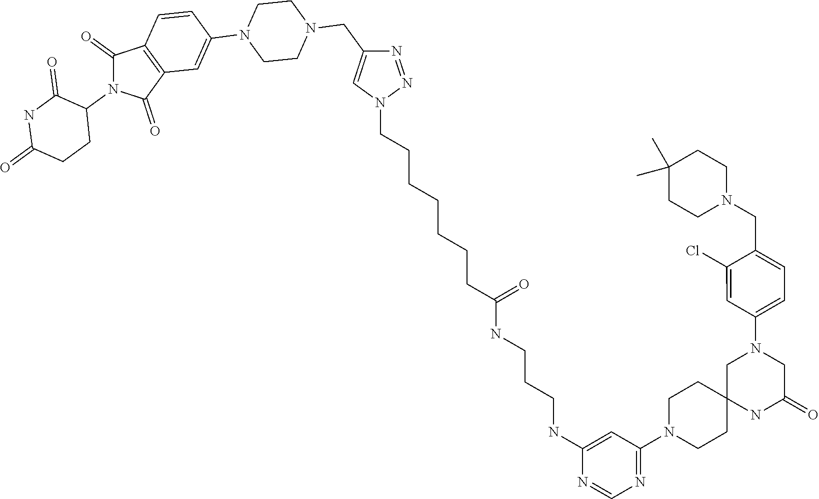 [SMILES: CC1(C)CCN(Cc2ccc(N3CC(=O)NC4(CCN(c5cc(NCCCNC(=O)CCCCCCCn6cc(CN7CCN(c8ccc9c(c8)C(=O)N(C8CCC(=O)NC8=O)C9=O)CC7)nn6)ncn5)CC4)C3)cc2Cl)CC1] |
| 287 | 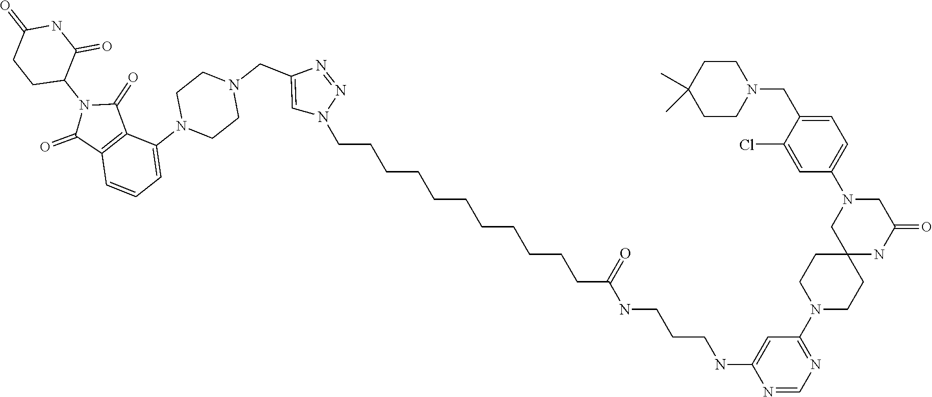 [SMILES: CC1(C)CCN(Cc2ccc(N3CC(=O)NC4(CCN(c5cc(NCCCNC(=O)CCCCCCCCCCCn6cc(CN7CCN(c8cccc9c8C(=O)N(C8CCC(=O)NC8=O)C9=O)CC7)nn6)ncn5)CC4)C3)cc2Cl)CC1] |
| 288 | 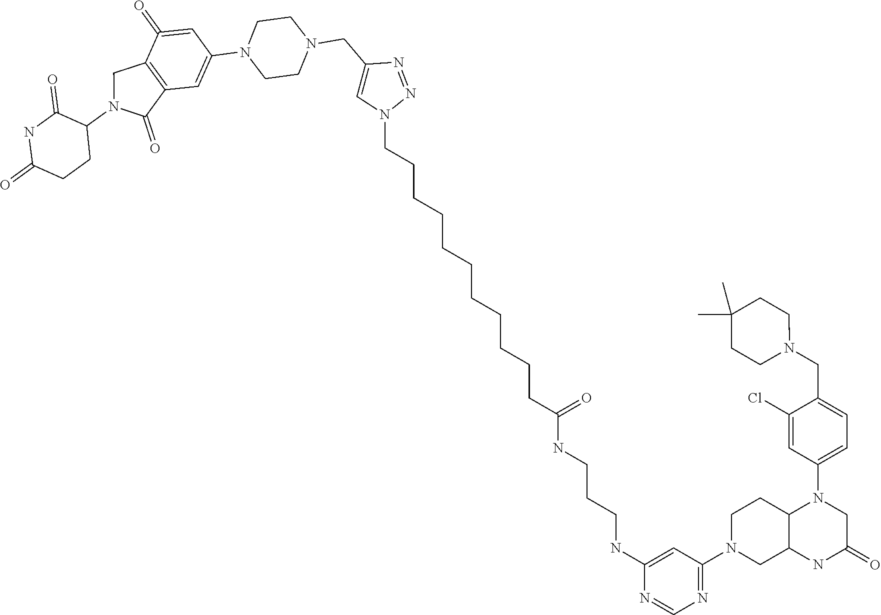 |
| 289 | 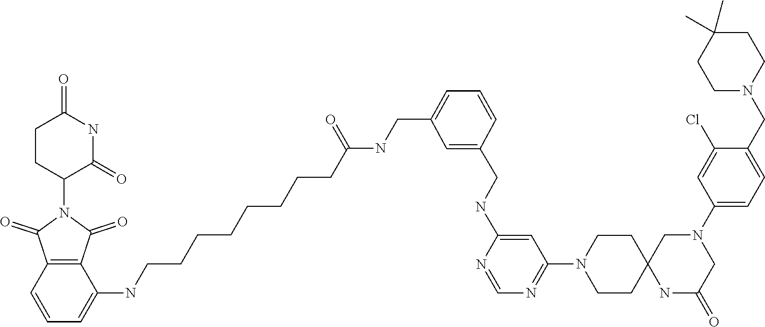 [SMILES: CC1(C)CCN(Cc2ccc(N3CC(=O)NC4(CCN(c5cc(NCc6cccc(CNC(=O)CCCCCCCCNc7cccc8c7C(=O)N(C7CCC(=O)NC7=O)C8=O)c6)ncn5)CC4)C3)cc2Cl)CC1] |
| 290 | 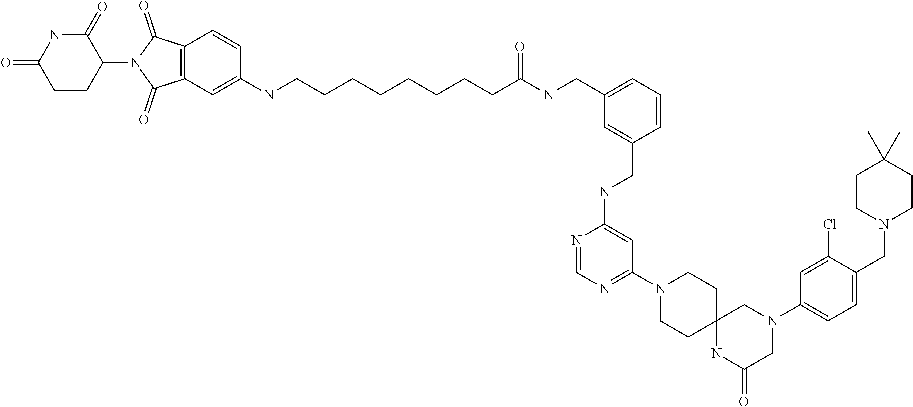 [SMILES: CC1(C)CCN(Cc2ccc(N3CC(=O)NC4(CCN(c5cc(NCc6cccc(CNC(=O)CCCCCCCCNc7ccc8c(c7)C(=O)N(C7CCC(=O)NC7=O)C8=O)c6)ncn5)CC4)C3)cc2Cl)CC1] |
| 291 | 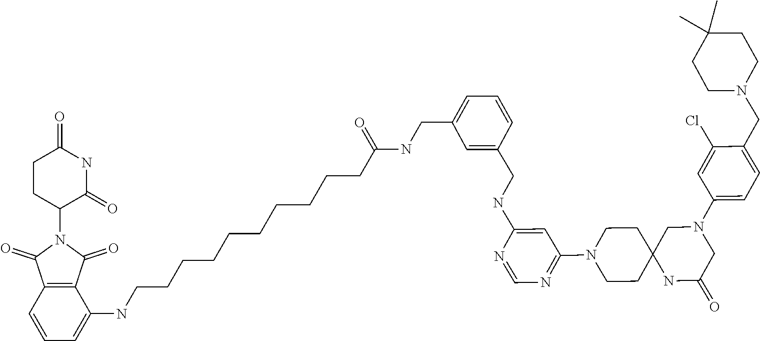 [SMILES: CC1(C)CCN(Cc2ccc(N3CC(=O)NC4(CCN(c5cc(NCc6cccc(CNC(=O)CCCCCCCCCCNc7cccc8c7C(=O)N(C7CCC(=O)NC7=O)C8=O)c6)ncn5)CC4)C3)cc2Cl)CC1] |
| 292 | 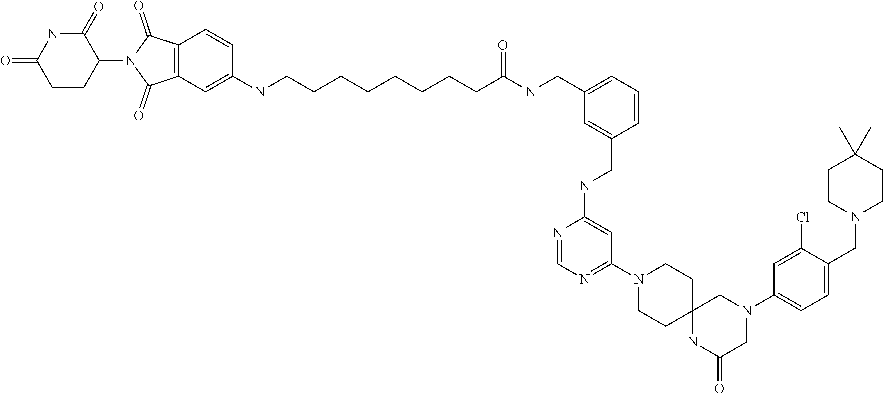 [SMILES: CC1(C)CCN(Cc2ccc(N3CC(=O)NC4(CCN(c5cc(NCc6cccc(CNC(=O)CCCCCCCCNc7ccc8c(c7)C(=O)N(C7CCC(=O)NC7=O)C8=O)c6)ncn5)CC4)C3)cc2Cl)CC1] |
| 293 | 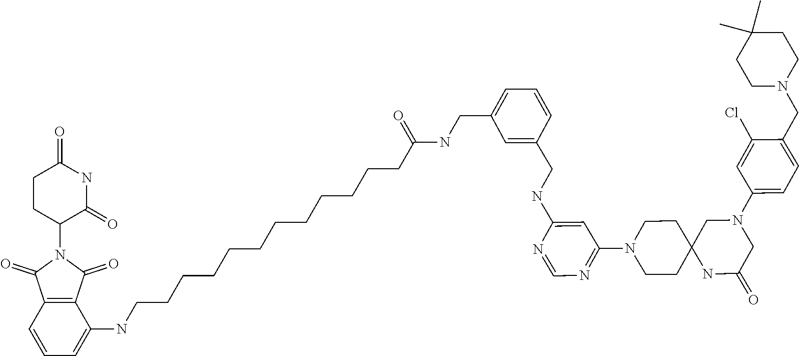 [SMILES: CC1(C)CCN(Cc2ccc(N3CC(=O)NC4(CCN(c5cc(NCc6cccc(CNC(=O)CCCCCCCCCCCCNc7cccc8c7C(=O)N(C7CCC(=O)NC7=O)C8=O)c6)ncn5)CC4)C3)cc2Cl)CC1] |
| 294 | 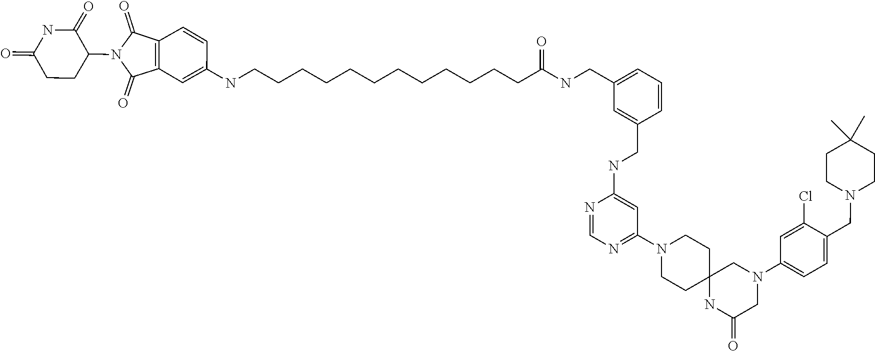 [SMILES: CC1(C)CCN(Cc2ccc(N3CC(=O)NC4(CCN(c5cc(NCc6cccc(CNC(=O)CCCCCCCCCCCCNc7ccc8c(c7)C(=O)N(C7CCC(=O)NC7=O)C8=O)c6)ncn5)CC4)C3)cc2Cl)CC1] |
| 295 | 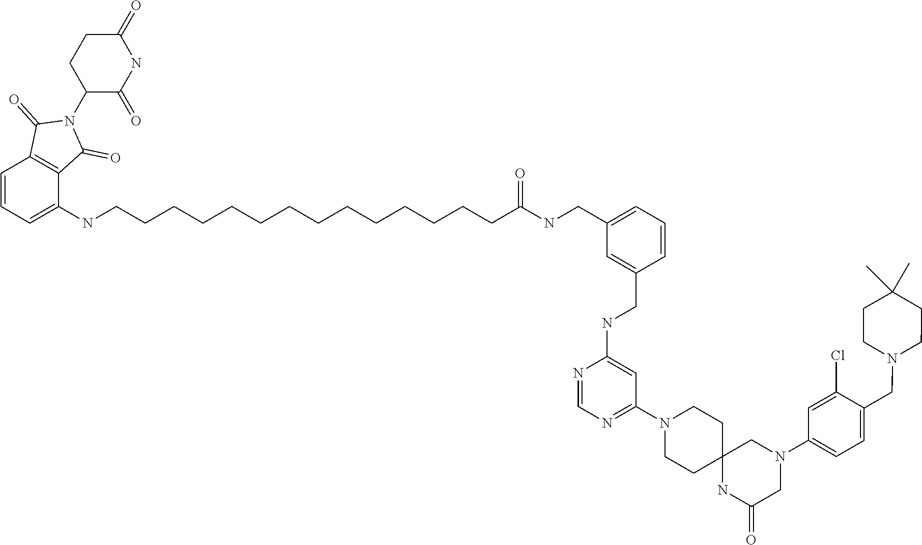 [SMILES: CC1(C)CCN(Cc2ccc(N3CC(=O)NC4(CCN(c5cc(NCc6cccc(CNC(=O)CCCCCCCCCCCCCCNc7cccc8c7C(=O)N(C7CCC(=O)NC7=O)C8=O)c6)ncn5)CC4)C3)cc2Cl)CC1] |
| 296 | 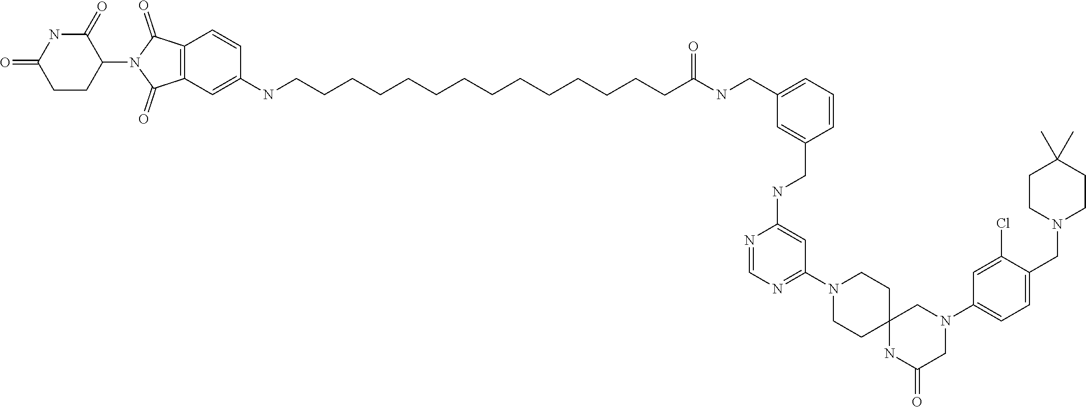 [SMILES: CC1(C)CCN(Cc2ccc(N3CC(=O)NC4(CCN(c5cc(NCc6cccc(CNC(=O)CCCCCCCCCCCCCCNc7ccc8c(c7)C(=O)N(C7CCC(=O)NC7=O)C8=O)c6)ncn5)CC4)C3)cc2Cl)CC1] |
| 297 | 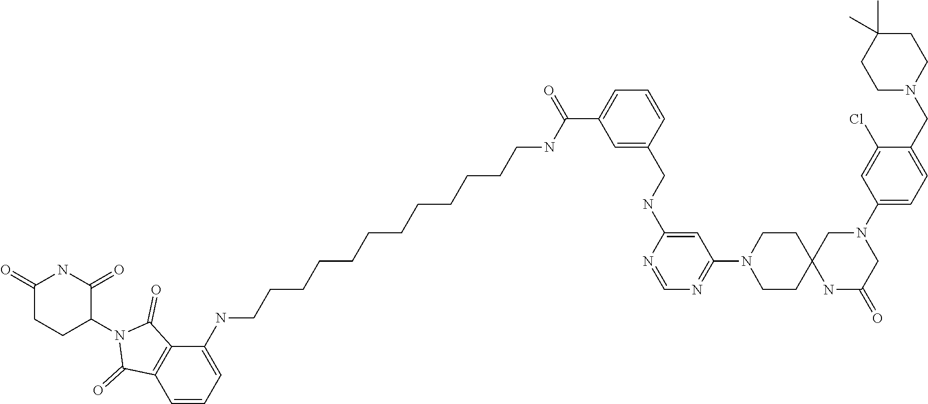 [SMILES: CC1(C)CCN(Cc2ccc(N3CC(=O)NC4(CCN(c5cc(NCc6cccc(C(=O)NCCCCCCCCCCCCNc7cccc8c7C(=O)N(C7CCC(=O)NC7=O)C8=O)c6)ncn5)CC4)C3)cc2Cl)CC1] |
| 298 | 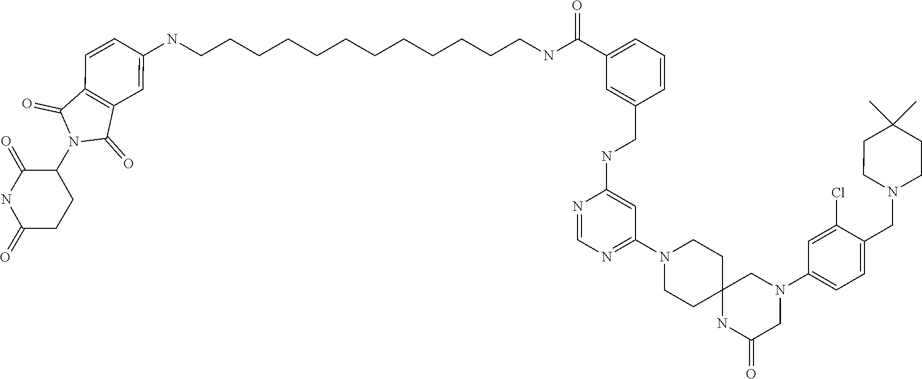 [SMILES: CC1(C)CCN(Cc2ccc(N3CC(=O)NC4(CCN(c5cc(NCc6cccc(C(=O)NCCCCCCCCCCCCNc7ccc8c(c7)C(=O)N(C7CCC(=O)NC7=O)C8=O)c6)ncn5)CC4)C3)cc2Cl)CC1] |
| 299 | 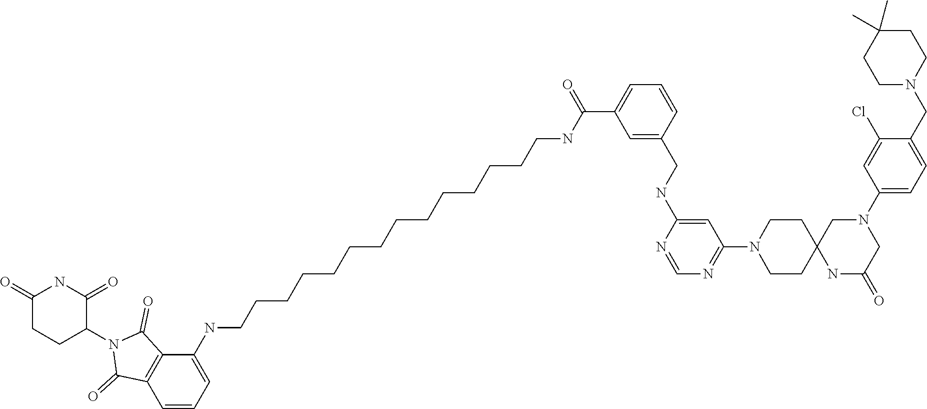 [SMILES: CC1(C)CCN(Cc2ccc(N3CC(=O)NC4(CCN(c5cc(NCc6cccc(C(=O)NCCCCCCCCCCCCCCNc7cccc8c7C(=O)N(C7CCC(=O)NC7=O)C8=O)c6)ncn5)CC4)C3)cc2Cl)CC1] |
| 300 | 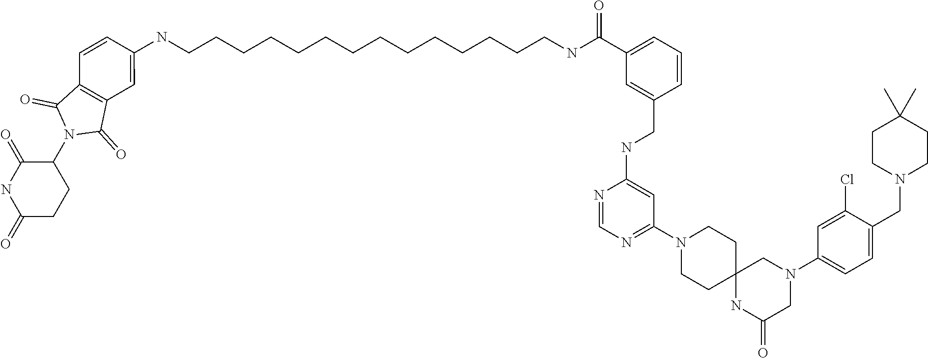 [SMILES: CC1(C)CCN(Cc2ccc(N3CC(=O)NC4(CCN(c5cc(NCc6cccc(C(=O)NCCCCCCCCCCCCCCNc7ccc8c(c7)C(=O)N(C7CCC(=O)NC7=O)C8=O)c6)ncn5)CC4)C3)cc2Cl)CC1] |
| 301 | 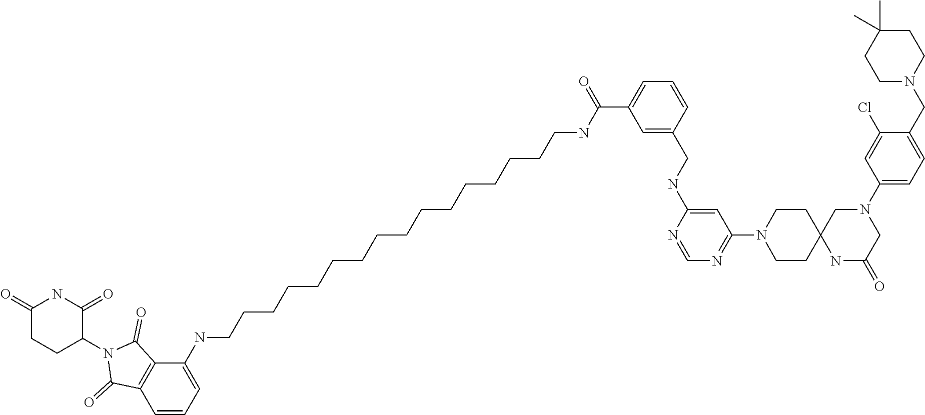 [SMILES: CC1(C)CCN(Cc2ccc(N3CC(=O)NC4(CCN(c5cc(NCc6cccc(C(=O)NCCCCCCCCCCCCCCCCNc7cccc8c7C(=O)N(C7CCC(=O)NC7=O)C8=O)c6)ncn5)CC4)C3)cc2Cl)CC1] |
| 302 | 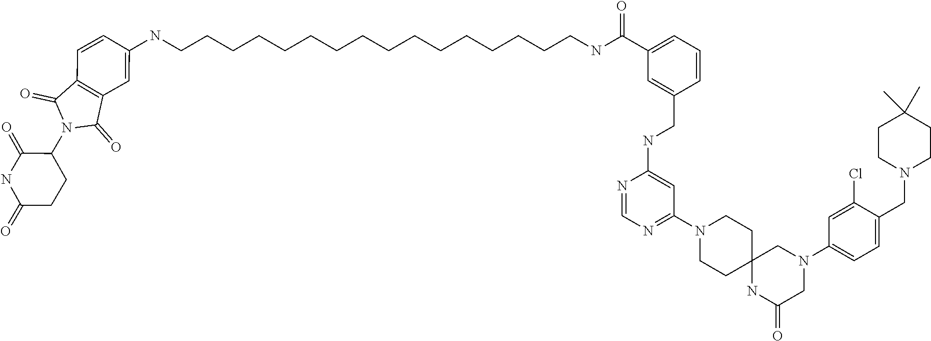 [SMILES: CC1(C)CCN(Cc2ccc(N3CC(=O)NC4(CCN(c5cc(NCc6cccc(C(=O)NCCCCCCCCCCCCCCCCNc7ccc8c(c7)C(=O)N(C7CCC(=O)NC7=O)C8=O)c6)ncn5)CC4)C3)cc2Cl)CC1] |
| 303 | 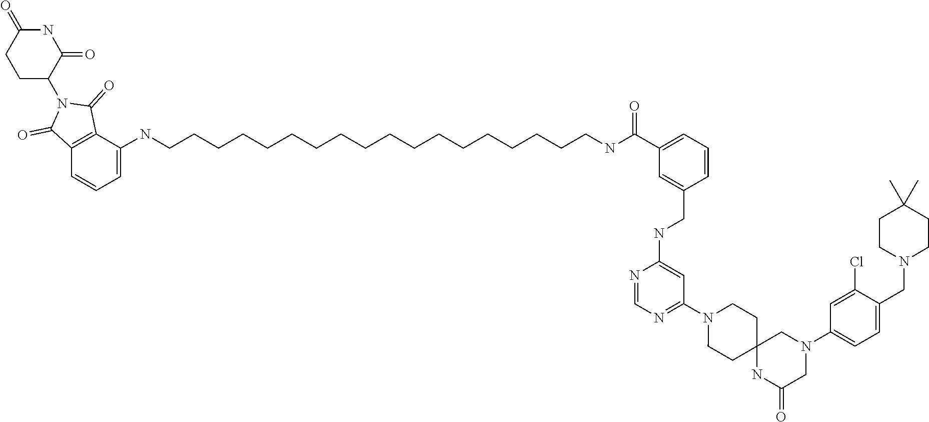 [SMILES: CC1(C)CCN(Cc2ccc(N3CC(=O)NC4(CCN(c5cc(NCc6cccc(C(=O)NCCCCCCCCCCCCCCCCCCNc7cccc8c7C(=O)N(C7CCC(=O)NC7=O)C8=O)c6)ncn5)CC4)C3)cc2Cl)CC1] |
| 304 | 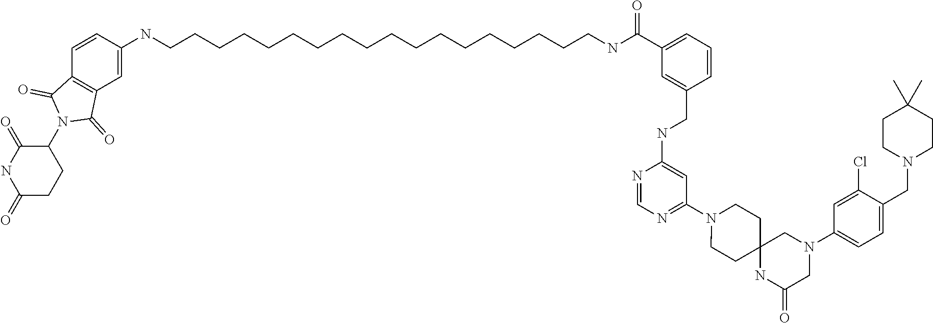 [SMILES: CC1(C)CCN(Cc2ccc(N3CC(=O)NC4(CCN(c5cc(NCc6cccc(C(=O)NCCCCCCCCCCCCCCCCCCNc7ccc8c(c7)C(=O)N(C7CCC(=O)NC7=O)C8=O)c6)ncn5)CC4)C3)cc2Cl)CC1] |
| 305 | 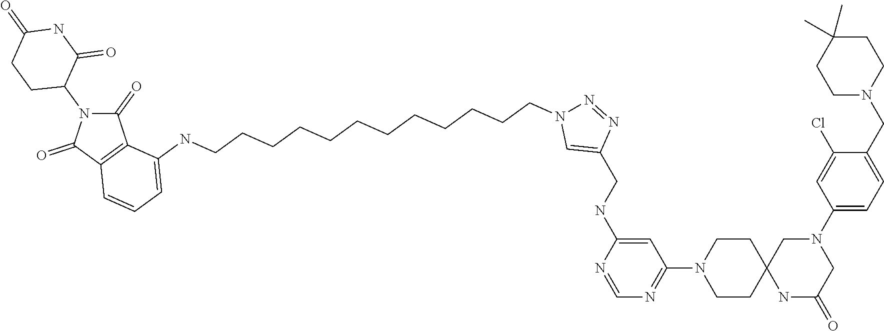 [SMILES: CC1(C)CCN(Cc2ccc(N3CC(=O)NC4(CCN(c5cc(NCc6cn(CCCCCCCCCCCCNc7cccc8c7C(=O)N(C7CCC(=O)NC7=O)C8=O)nn6)ncn5)CC4)C3)cc2Cl)CC1] |
| 306 | 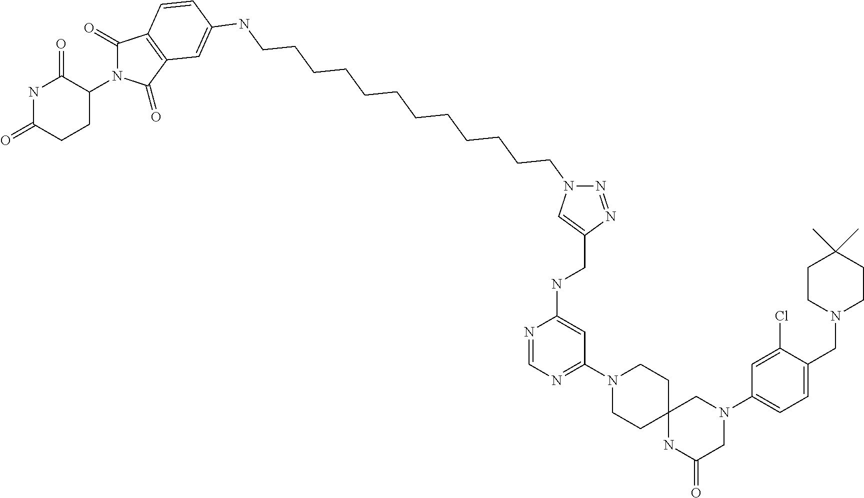 [SMILES: CC1(C)CCN(Cc2ccc(N3CC(=O)NC4(CCN(c5cc(NCc6cn(CCCCCCCCCCCCNc7ccc8c(c7)C(=O)N(C7CCC(=O)NC7=O)C8=O)nn6)ncn5)CC4)C3)cc2Cl)CC1] |
| 307 | 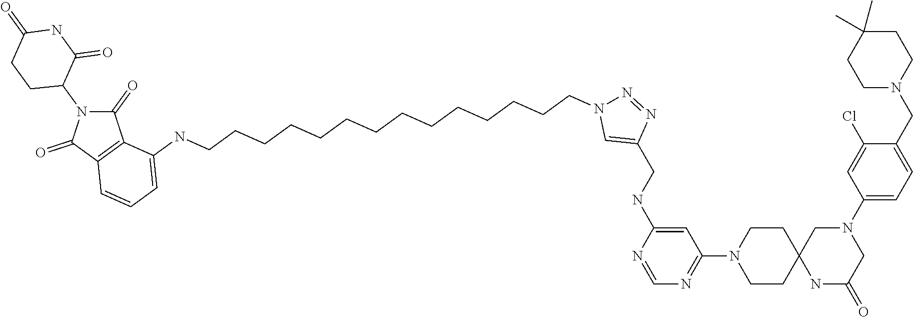 [SMILES: CC1(C)CCN(Cc2ccc(N3CC(=O)NC4(CCN(c5cc(NCc6cn(CCCCCCCCCCCCCCNc7cccc8c7C(=O)N(C7CCC(=O)NC7=O)C8=O)nn6)ncn5)CC4)C3)cc2Cl)CC1] |
| 308 | 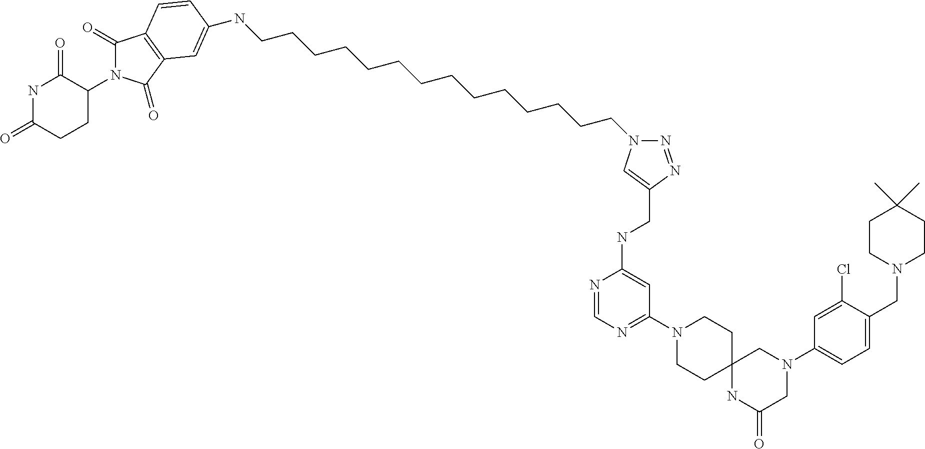 [SMILES: CC1(C)CCN(Cc2ccc(N3CC(=O)NC4(CCN(c5cc(NCc6cn(CCCCCCCCCCCCCCNc7ccc8c(c7)C(=O)N(C7CCC(=O)NC7=O)C8=O)nn6)ncn5)CC4)C3)cc2Cl)CC1] |
| 309 | 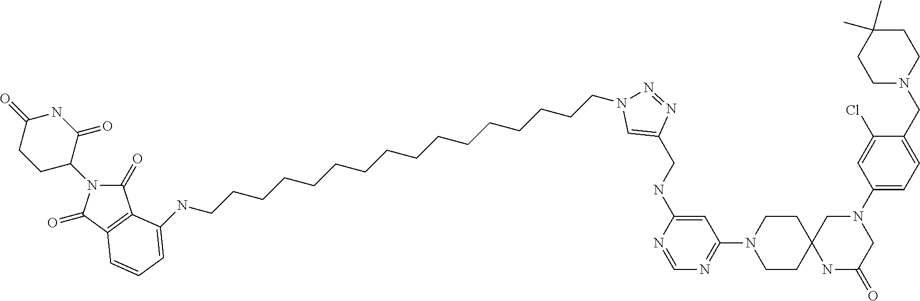 [SMILES: CC1(C)CCN(Cc2ccc(N3CC(=O)NC4(CCN(c5cc(NCc6cn(CCCCCCCCCCCCCCCCNc7cccc8c7C(=O)N(C7CCC(=O)NC7=O)C8=O)nn6)ncn5)CC4)C3)cc2Cl)CC1] |
| 310 | 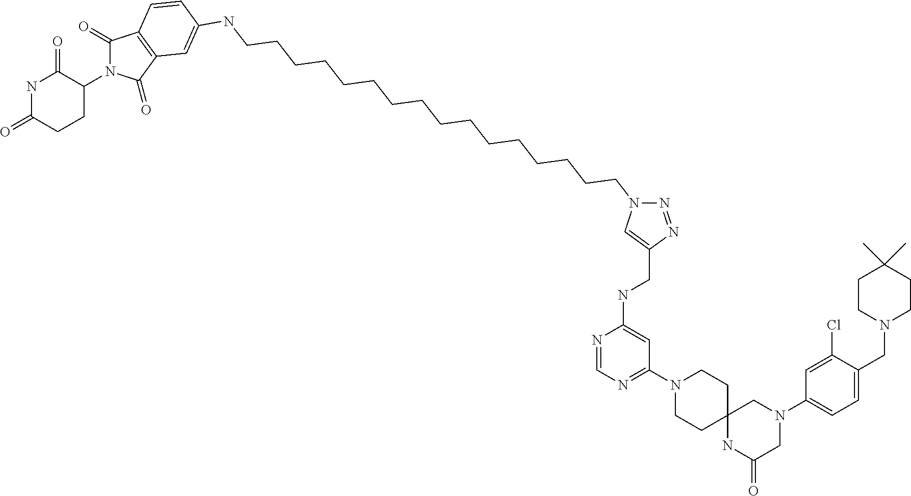 [SMILES: CC1(C)CCN(Cc2ccc(N3CC(=O)NC4(CCN(c5cc(NCc6cn(CCCCCCCCCCCCCCCCNc7ccc8c(c7)C(=O)N(C7CCC(=O)NC7=O)C8=O)nn6)ncn5)CC4)C3)cc2Cl)CC1] |
| 311 | 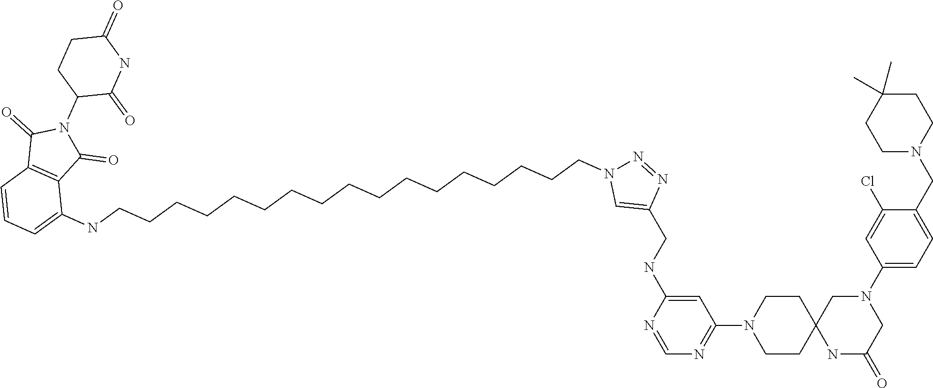 [SMILES: CC1(C)CCN(Cc2ccc(N3CC(=O)NC4(CCN(c5cc(NCc6cn(CCCCCCCCCCCCCCCCCNc7cccc8c7C(=O)N(C7CCC(=O)NC7=O)C8=O)nn6)ncn5)CC4)C3)cc2Cl)CC1] |
| 312 | 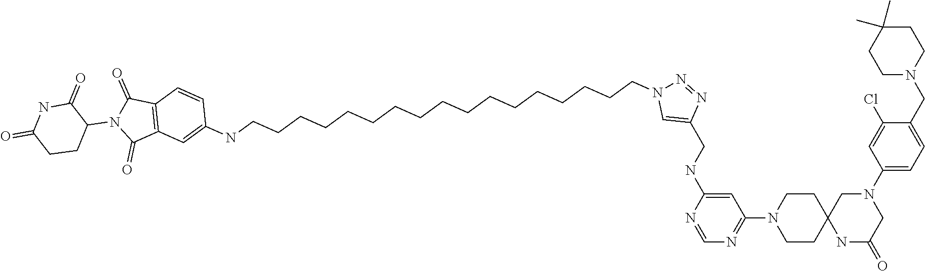 [SMILES: CC1(C)CCN(Cc2ccc(N3CC(=O)NC4(CCN(c5cc(NCc6cn(CCCCCCCCCCCCCCCCCNc7ccc8c(c7)C(=O)N(C7CCC(=O)NC7=O)C8=O)nn6)ncn5)CC4)C3)cc2Cl)CC1] |
| 313 | 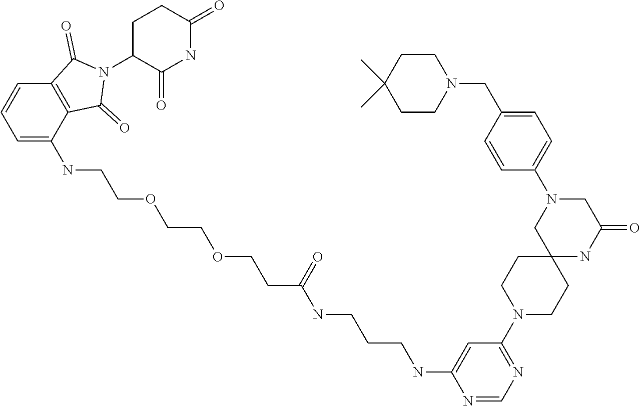 [SMILES: CC1(C)CCN(Cc2ccc(N3CC(=O)NC4(CCN(c5cc(NCCCNC(=O)CCOCCOCCNc6cccc7c6C(=O)N(C6CCC(=O)NC6=O)C7=O)ncn5)CC4)C3)cc2)CC1] |
| 314 | 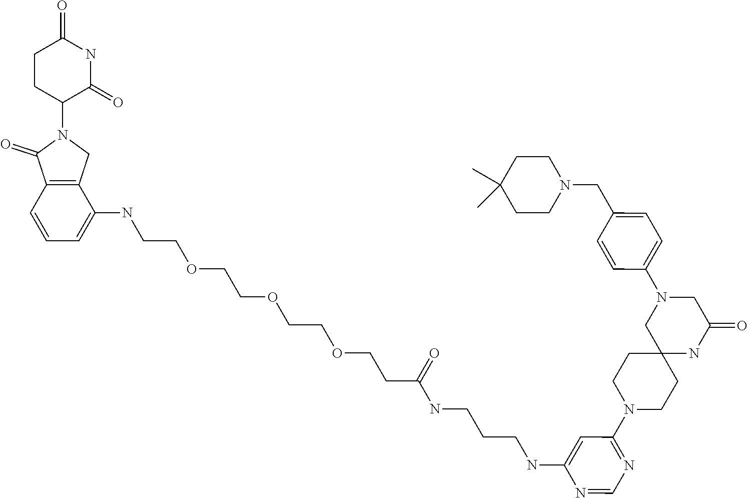 [SMILES: CC1(C)CCN(Cc2ccc(N3CC(=O)NC4(CCN(c5cc(NCCCNC(=O)CCOCCOCCOCCNc6cccc7c6CN(C6CCC(=O)NC6=O)C7=O)ncn5)CC4)C3)cc2)CC1] |
| 315 | 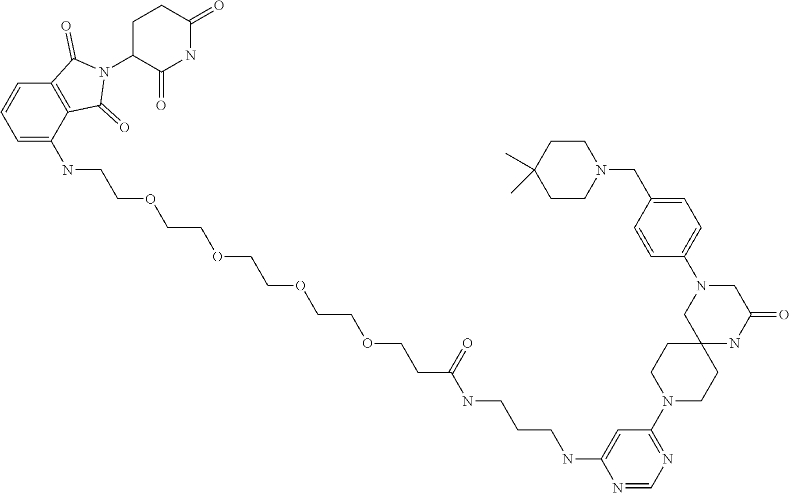 [SMILES: CC1(C)CCN(Cc2ccc(N3CC(=O)NC4(CCN(c5cc(NCCCNC(=O)CCOCCOCCOCCOCCNc6cccc7c6C(=O)N(C6CCC(=O)NC6=O)C7=O)ncn5)CC4)C3)cc2)CC1] |
| 316 | 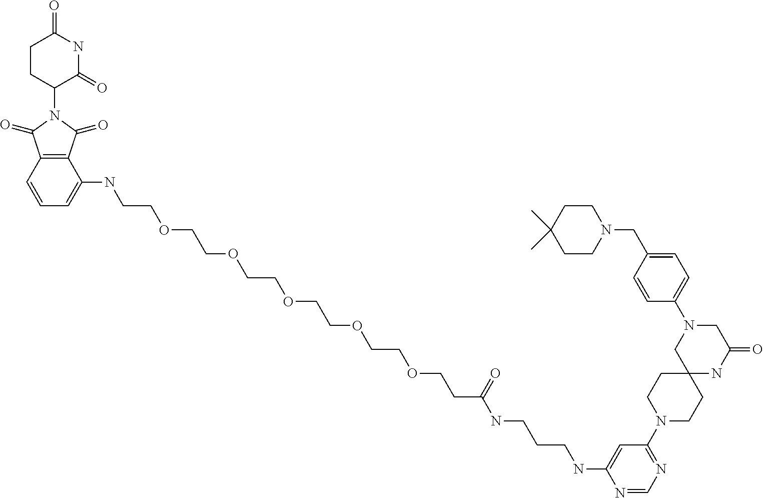 [SMILES: CC1(C)CCN(Cc2ccc(N3CC(=O)NC4(CCN(c5cc(NCCCNC(=O)CCOCCOCCOCCOCCOCCNc6cccc7c6C(=O)N(C6CCC(=O)NC6=O)C7=O)ncn5)CC4)C3)cc2)CC1] |
| 317 | 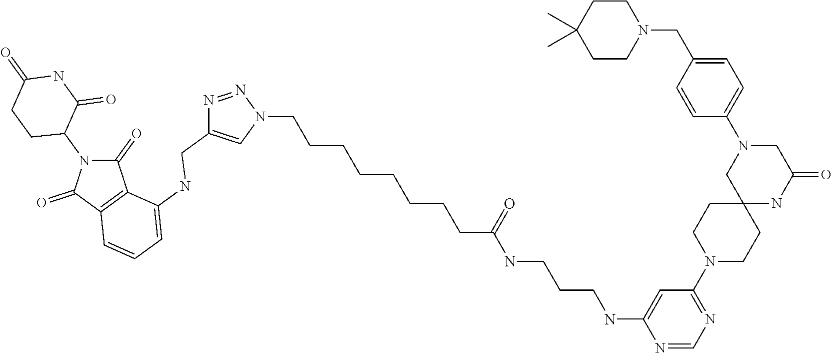 [SMILES: CC1(C)CCN(Cc2ccc(N3CC(=O)NC4(CCN(c5cc(NCCCNC(=O)CCCCCCCCn6cc(CNc7cccc8c7C(=O)N(C7CCC(=O)NC7=O)C8=O)nn6)ncn5)CC4)C3)cc2)CC1] |
| 318 | 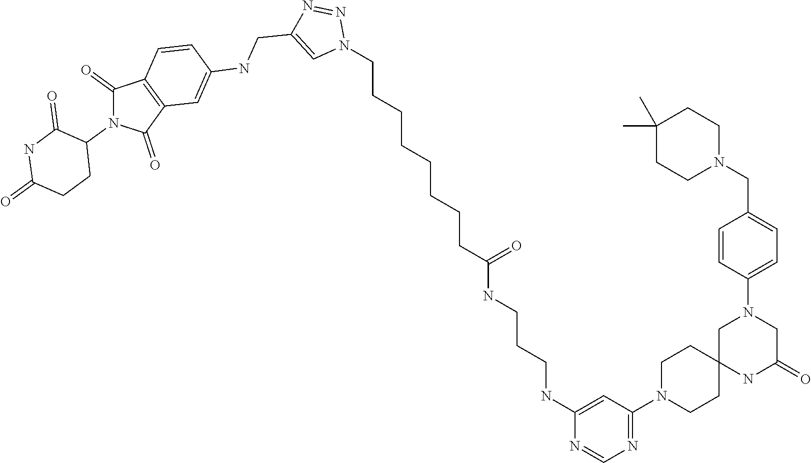 [SMILES: CC1(C)CCN(Cc2ccc(N3CC(=O)NC4(CCN(c5cc(NCCCNC(=O)CCCCCCCCn6cc(CNc7ccc8c(c7)C(=O)N(C7CCC(=O)NC7=O)C8=O)nn6)ncn5)CC4)C3)cc2)CC1] |
| 319 | 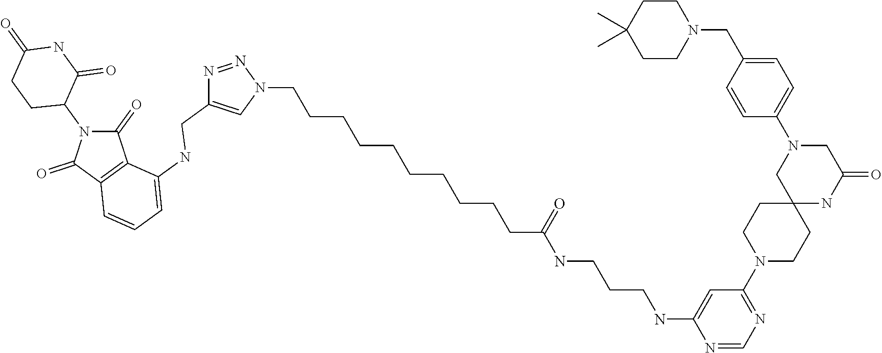 [SMILES: CC1(C)CCN(Cc2ccc(N3CC(=O)NC4(CCN(c5cc(NCCCNC(=O)CCCCCCCCCCn6cc(CNc7cccc8c7C(=O)N(C7CCC(=O)NC7=O)C8=O)nn6)ncn5)CC4)C3)cc2)CC1] |
| 320 | 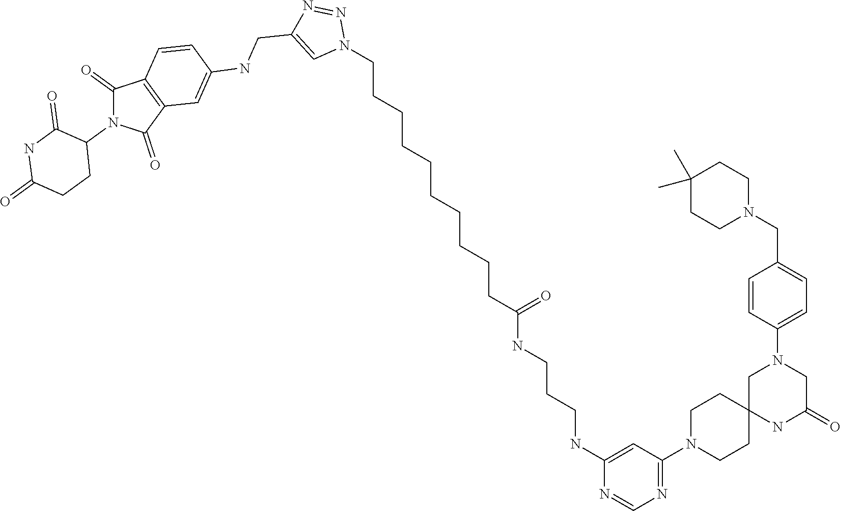 [SMILES: CC1(C)CCN(Cc2ccc(N3CC(=O)NC4(CCN(c5cc(NCCCNC(=O)CCCCCCCCCCn6cc(CNc7ccc8c(c7)C(=O)N(C7CCC(=O)NC7=O)C8=O)nn6)ncn5)CC4)C3)cc2)CC1] |
| 321 | 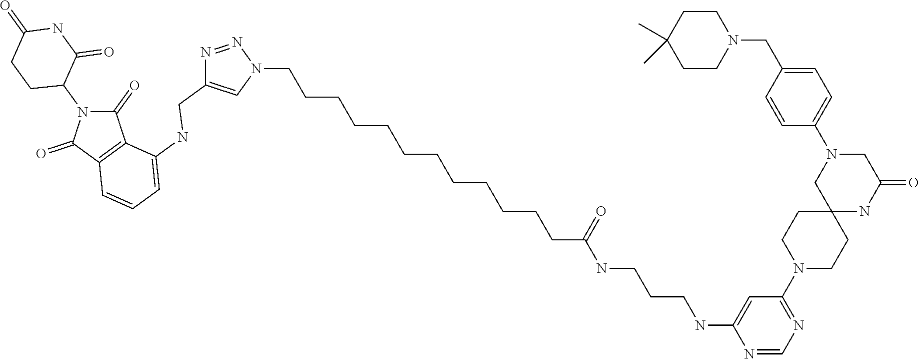 [SMILES: CC1(C)CCN(Cc2ccc(N3CC(=O)NC4(CCN(c5cc(NCCCNC(=O)CCCCCCCCCCCCn6cc(CNc7cccc8c7C(=O)N(C7CCC(=O)NC7=O)C8=O)nn6)ncn5)CC4)C3)cc2)CC1] |
| 322 | 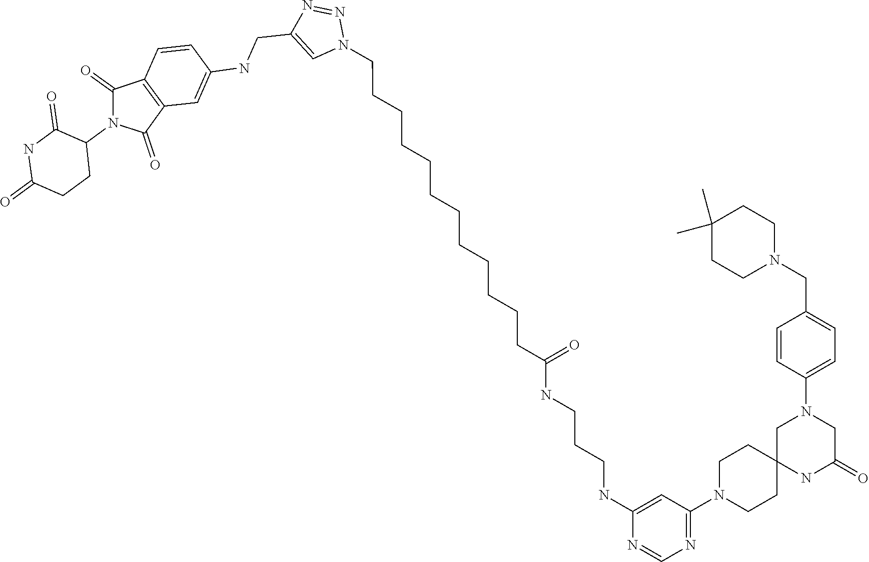 [SMILES: CC1(C)CCN(Cc2ccc(N3CC(=O)NC4(CCN(c5cc(NCCCNC(=O)CCCCCCCCCCCCn6cc(CNc7ccc8c(c7)C(=O)N(C7CCC(=O)NC7=O)C8=O)nn6)ncn5)CC4)C3)cc2)CC1] |
| 323 | 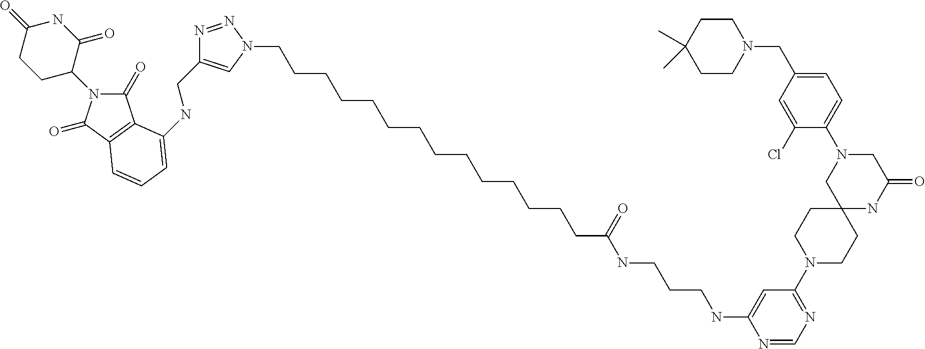 [SMILES: CC1(C)CCN(Cc2ccc(N3CC(=O)NC4(CCN(c5cc(NCCCNC(=O)CCCCCCCCCCCCCCn6cc(CNc7cccc8c7C(=O)N(C7CCC(=O)NC7=O)C8=O)nn6)ncn5)CC4)C3)c(Cl)c2)CC1] |
| 324 | 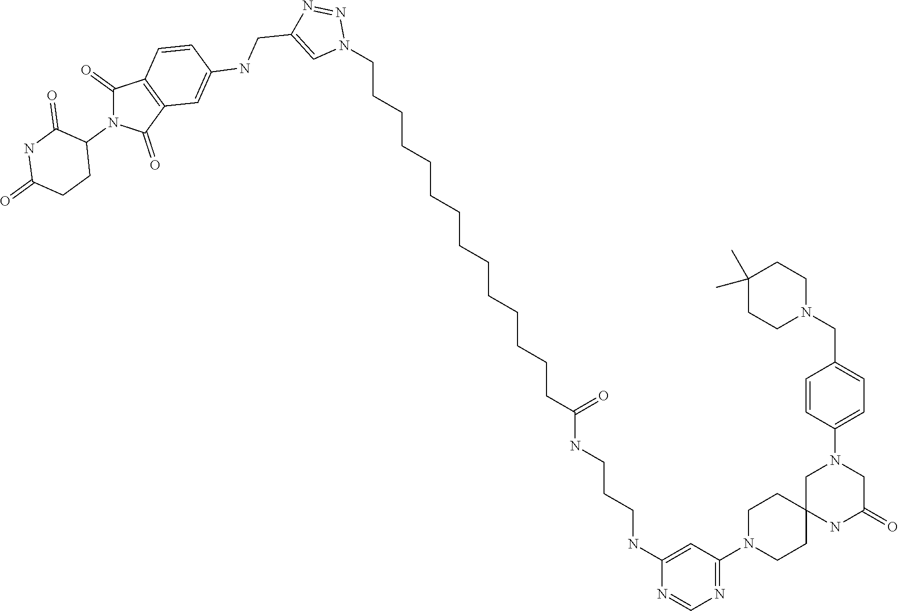 [SMILES: CC1(C)CCN(Cc2ccc(N3CC(=O)NC4(CCN(c5cc(NCCCNC(=O)CCCCCCCCCCCCCCn6cc(CNc7ccc8c(c7)C(=O)N(C7CCC(=O)NC7=O)C8=O)nn6)ncn5)CC4)C3)cc2)CC1] |
| 325 | 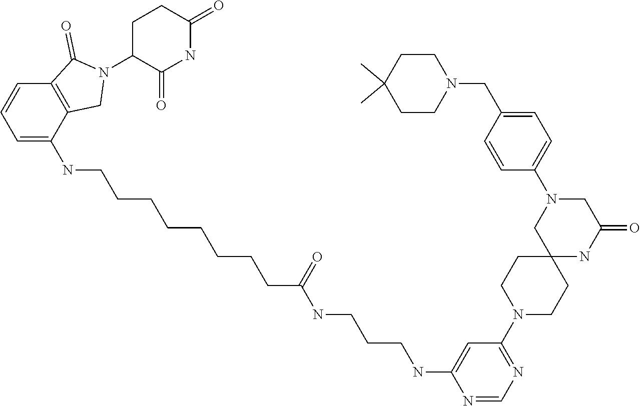 [SMILES: CC1(C)CCN(Cc2ccc(N3CC(=O)NC4(CCN(c5cc(NCCCNC(=O)CCCCCCCCNc6cccc7c6CN(C6CCC(=O)NC6=O)C7=O)ncn5)CC4)C3)cc2)CC1] |
| 326 | 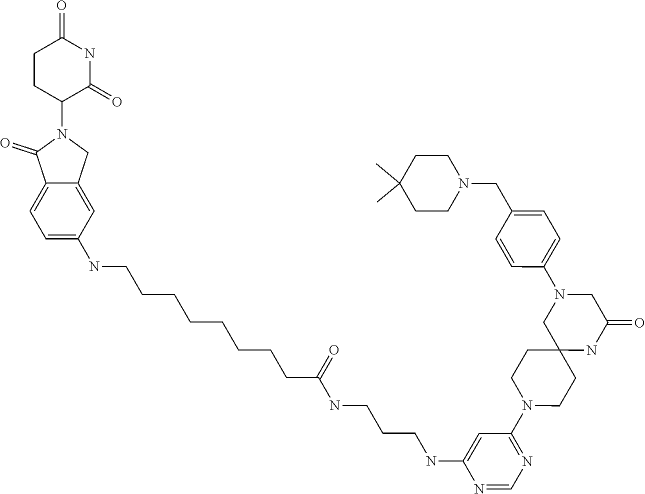 [SMILES: CC1(C)CCN(Cc2ccc(N3CC(=O)NC4(CCN(c5cc(NCCCNC(=O)CCCCCCCCNc6ccc7c(c6)CN(C6CCC(=O)NC6=O)C7=O)ncn5)CC4)C3)cc2)CC1] |
| 327 | 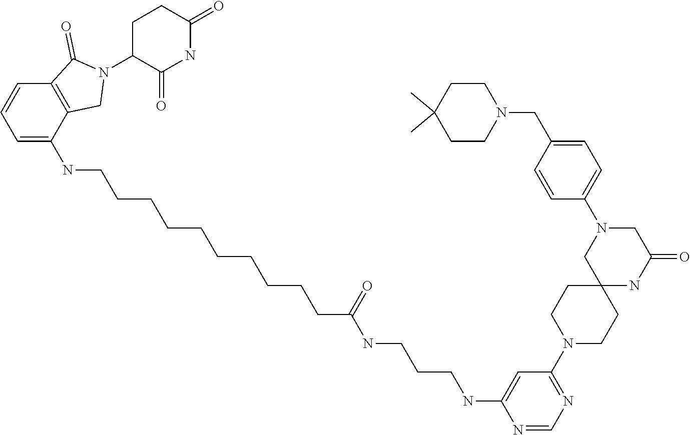 [SMILES: CC1(C)CCN(Cc2ccc(N3CC(=O)NC4(CCN(c5cc(NCCCNC(=O)CCCCCCCCCCNc6cccc7c6CN(C6CCC(=O)NC6=O)C7=O)ncn5)CC4)C3)cc2)CC1] |
| 328 | 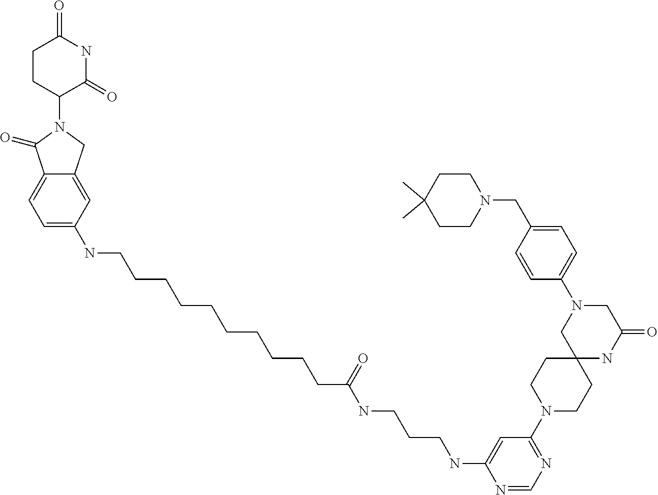 [SMILES: CC1(C)CCN(Cc2ccc(N3CC(=O)NC4(CCN(c5cc(NCCCNC(=O)CCCCCCCCCCNc6ccc7c(c6)CN(C6CCC(=O)NC6=O)C7=O)ncn5)CC4)C3)cc2)CC1] |
| 329 | 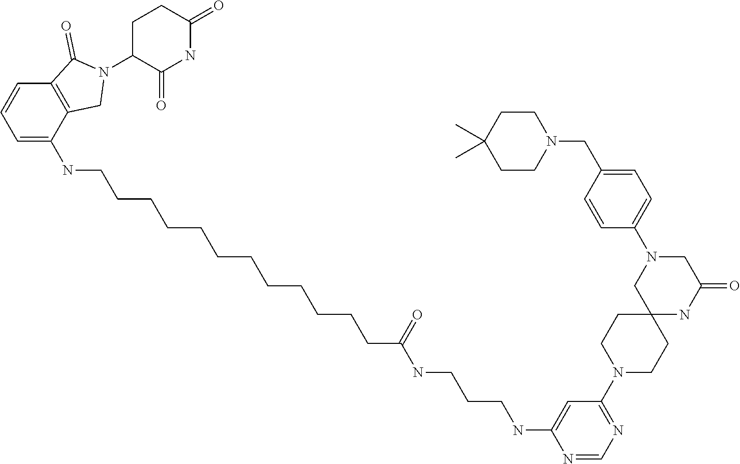 [SMILES: CC1(C)CCN(Cc2ccc(N3CC(=O)NC4(CCN(c5cc(NCCCNC(=O)CCCCCCCCCCCCNc6cccc7c6CN(C6CCC(=O)NC6=O)C7=O)ncn5)CC4)C3)cc2)CC1] |
| 330 | 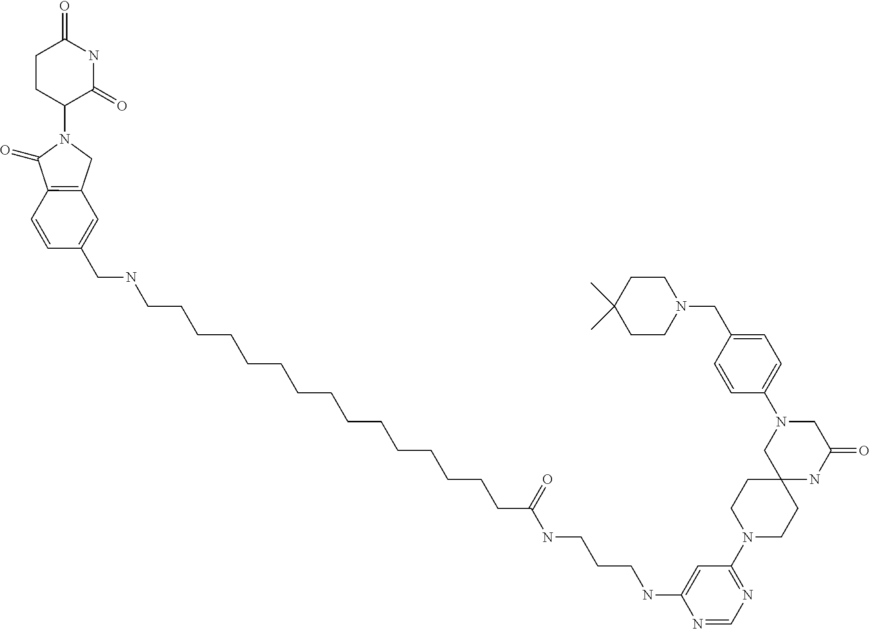 [SMILES: CC1(C)CCN(Cc2ccc(N3CC(=O)NC4(CCN(c5cc(NCCCNC(=O)CCCCCCCCCCCCCCCNCc6ccc7c(c6)CN(C6CCC(=O)NC6=O)C7=O)ncn5)CC4)C3)cc2)CC1] |
| 331 | 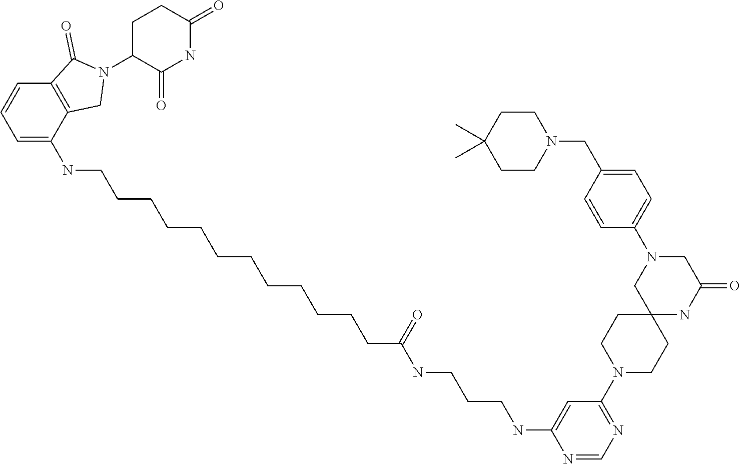 [SMILES: CC1(C)CCN(Cc2ccc(N3CC(=O)NC4(CCN(c5cc(NCCCNC(=O)CCCCCCCCCCCCNc6cccc7c6CN(C6CCC(=O)NC6=O)C7=O)ncn5)CC4)C3)cc2)CC1] |
| 332 | 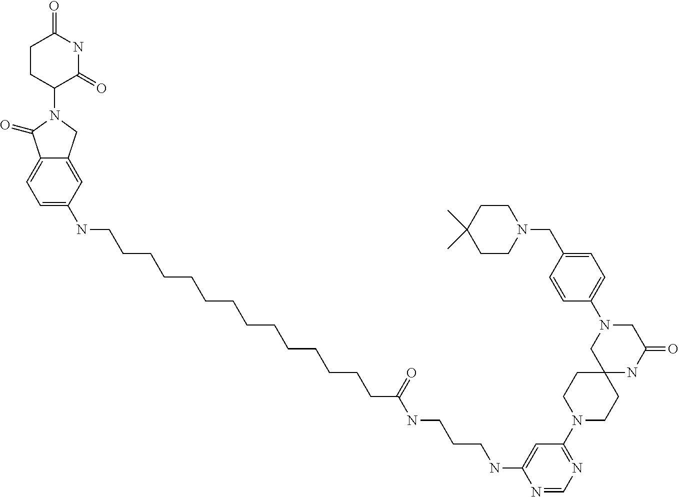 [SMILES: CC1(C)CCN(Cc2ccc(N3CC(=O)NC4(CCN(c5cc(NCCCNC(=O)CCCCCCCCCCCCCCNc6ccc7c(c6)CN(C6CCC(=O)NC6=O)C7=O)ncn5)CC4)C3)cc2)CC1] |
| 333 | 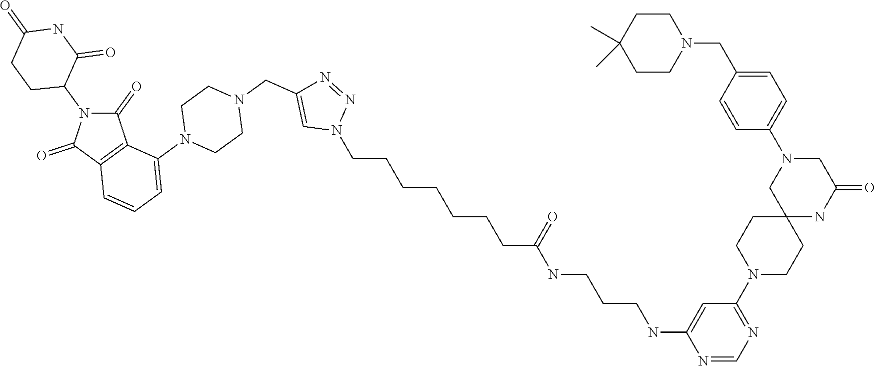 [SMILES: CC1(C)CCN(Cc2ccc(N3CC(=O)NC4(CCN(c5cc(NCCCNC(=O)CCCCCCCn6cc(CN7CCN(c8cccc9c8C(=O)N(C8CCC(=O)NC8=O)C9=O)CC7)nn6)ncn5)CC4)C3)cc2)CC1] |
| 334 | 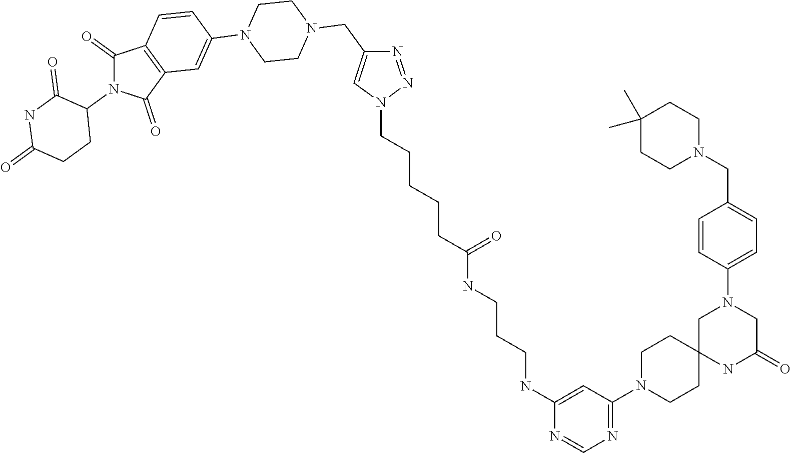 [SMILES: CC1(C)CCN(Cc2ccc(N3CC(=O)NC4(CCN(c5cc(NCCCNC(=O)CCCCCn6cc(CN7CCN(c8ccc9c(c8)C(=O)N(C8CCC(=O)NC8=O)C9=O)CC7)nn6)ncn5)CC4)C3)cc2)CC1] |
| 335 | 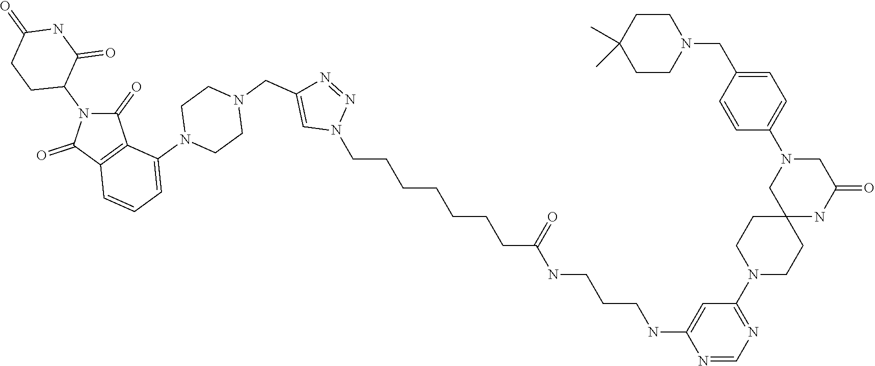 [SMILES: CC1(C)CCN(Cc2ccc(N3CC(=O)NC4(CCN(c5cc(NCCCNC(=O)CCCCCCCn6cc(CN7CCN(c8cccc9c8C(=O)N(C8CCC(=O)NC8=O)C9=O)CC7)nn6)ncn5)CC4)C3)cc2)CC1] |
| 336 | 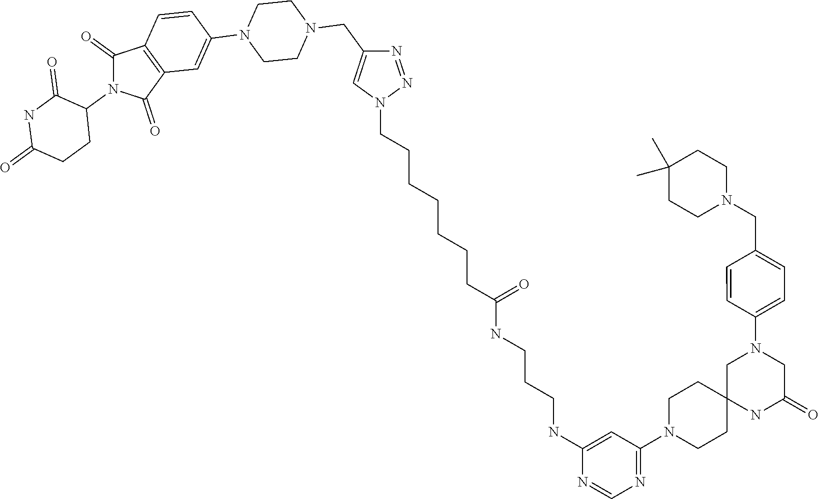 [SMILES: CC1(C)CCN(Cc2ccc(N3CC(=O)NC4(CCN(c5cc(NCCCNC(=O)CCCCCCCn6cc(CN7CCN(c8ccc9c(c8)C(=O)N(C8CCC(=O)NC8=O)C9=O)CC7)nn6)ncn5)CC4)C3)cc2)CC1] |
| 337 | 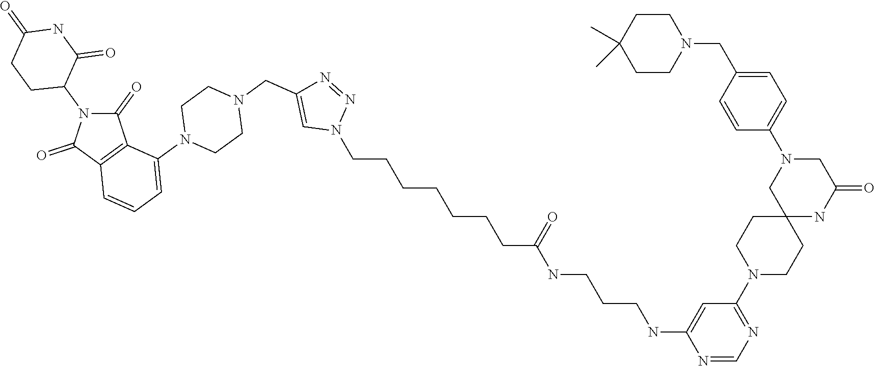 [SMILES: CC1(C)CCN(Cc2ccc(N3CC(=O)NC4(CCN(c5cc(NCCCNC(=O)CCCCCCCn6cc(CN7CCN(c8cccc9c8C(=O)N(C8CCC(=O)NC8=O)C9=O)CC7)nn6)ncn5)CC4)C3)cc2)CC1] |
| 338 | 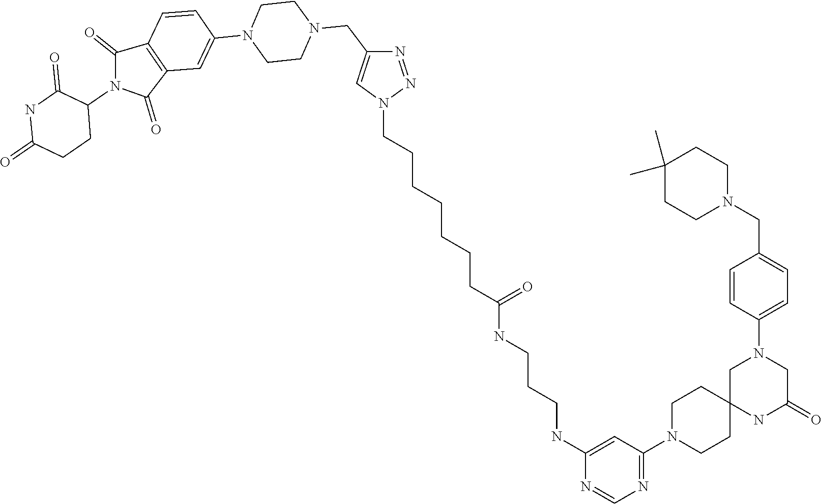 [SMILES: CC1(C)CCN(Cc2ccc(N3CC(=O)NC4(CCN(c5cc(NCCCNC(=O)CCCCCCCn6cc(CN7CCN(c8ccc9c(c8)C(=O)N(C8CCC(=O)NC8=O)C9=O)CC7)nn6)ncn5)CC4)C3)cc2)CC1] |
| 339 | 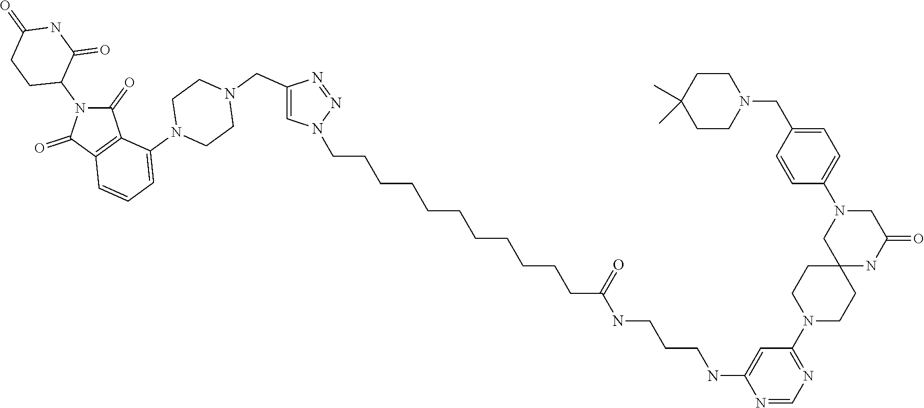 [SMILES: CC1(C)CCN(Cc2ccc(N3CC(=O)NC4(CCN(c5cc(NCCCNC(=O)CCCCCCCCCCCn6cc(CN7CCN(c8cccc9c8C(=O)N(C8CCC(=O)NC8=O)C9=O)CC7)nn6)ncn5)CC4)C3)cc2)CC1] |
| 340 | 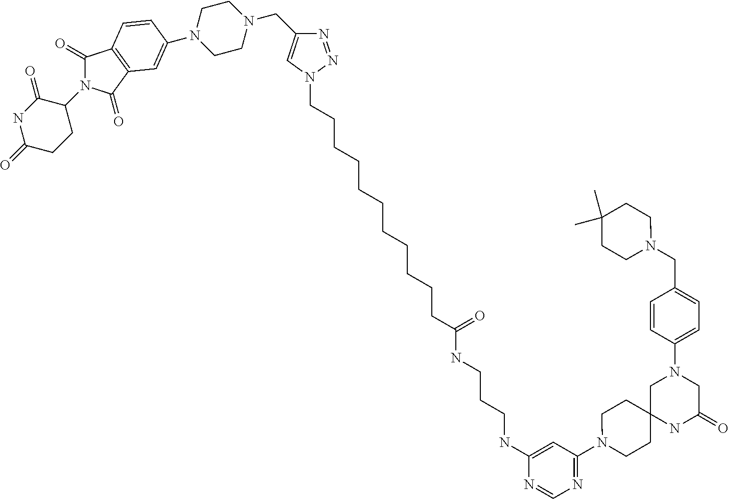 [SMILES: CC1(C)CCN(Cc2ccc(N3CC(=O)NC4(CCN(c5cc(NCCCNC(=O)CCCCCCCCCCCn6cc(CN7CCN(c8ccc9c(c8)C(=O)N(C8CCC(=O)NC8=O)C9=O)CC7)nn6)ncn5)CC4)C3)cc2)CC1] |
| 341 | 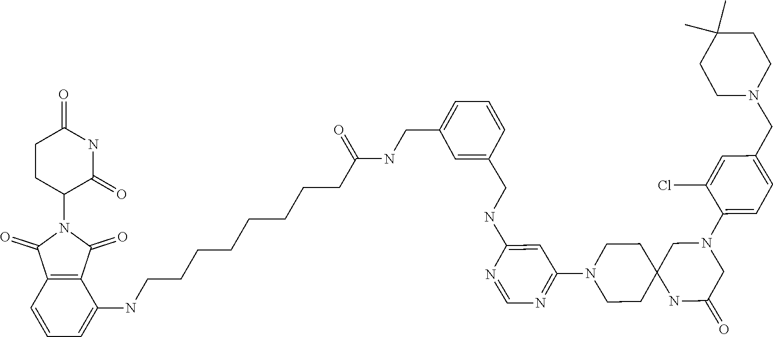 [SMILES: CC1(C)CCN(Cc2ccc(N3CC(=O)NC4(CCN(c5cc(NCc6cccc(CNC(=O)CCCCCCCCNc7cccc8c7C(=O)N(C7CCC(=O)NC7=O)C8=O)c6)ncn5)CC4)C3)c(Cl)c2)CC1] |
| 342 | 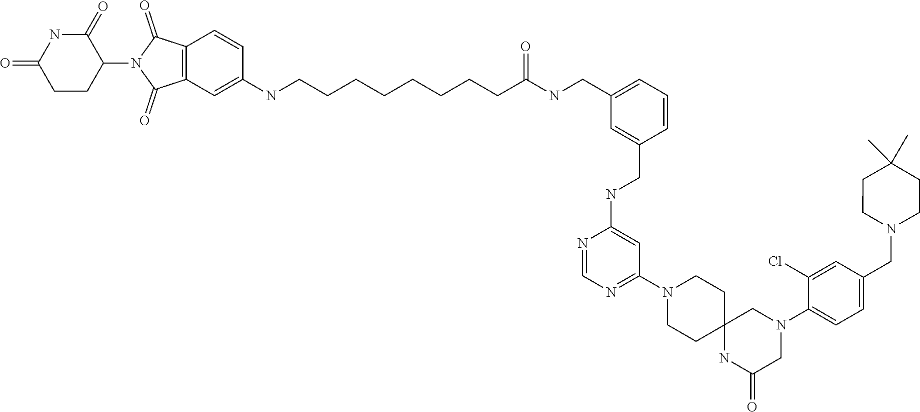 [SMILES: CC1(C)CCN(Cc2ccc(N3CC(=O)NC4(CCN(c5cc(NCc6cccc(CNC(=O)CCCCCCCCNc7ccc8c(c7)C(=O)N(C7CCC(=O)NC7=O)C8=O)c6)ncn5)CC4)C3)c(Cl)c2)CC1] |
| 343 | 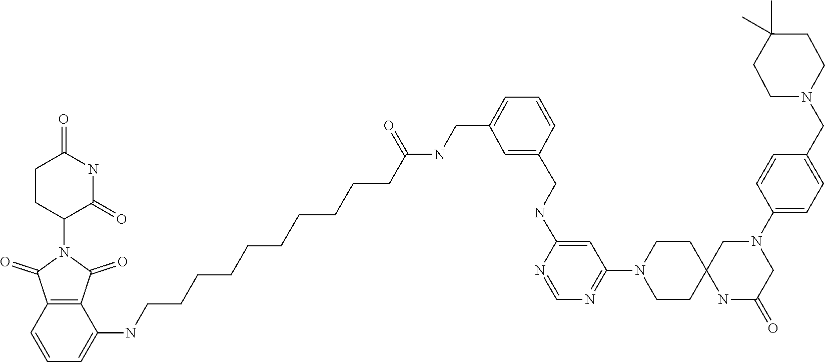 [SMILES: CC1(C)CCN(Cc2ccc(N3CC(=O)NC4(CCN(c5cc(NCc6cccc(CNC(=O)CCCCCCCCCCNc7cccc8c7C(=O)N(C7CCC(=O)NC7=O)C8=O)c6)ncn5)CC4)C3)cc2)CC1] |
| 344 | 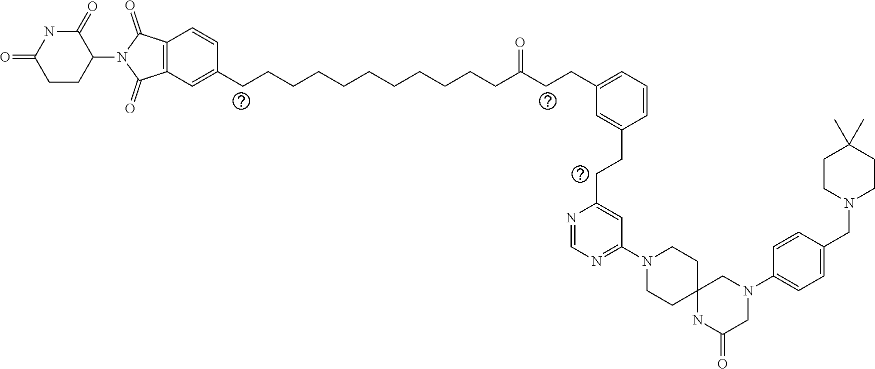 [SMILES: CC1(C)CCN(Cc2ccc(N3CC(=O)NC4(CCN(c5cc(CCc6cccc(CCC(=O)CCCCCCCCCCCc7ccc8c(c7)C(=O)N(C7CCC(=O)NC7=O)C8=O)c6)ncn5)CC4)C3)cc2)CC1] |
| 345 | 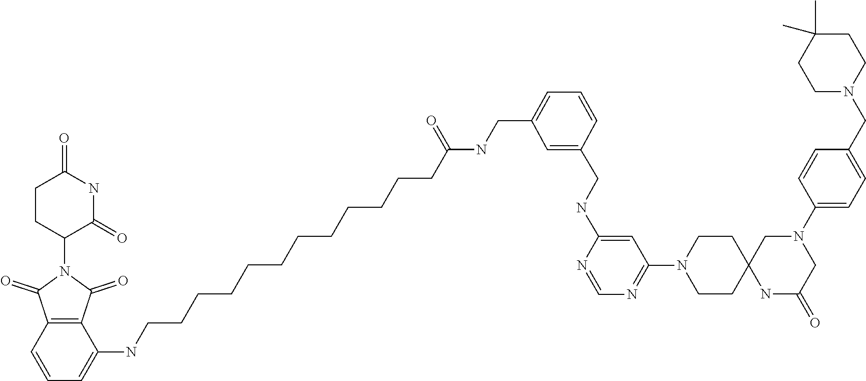 [SMILES: CC1(C)CCN(Cc2ccc(N3CC(=O)NC4(CCN(c5cc(NCc6cccc(CNC(=O)CCCCCCCCCCCCNc7cccc8c7C(=O)N(C7CCC(=O)NC7=O)C8=O)c6)ncn5)CC4)C3)cc2)CC1] |
| 346 | 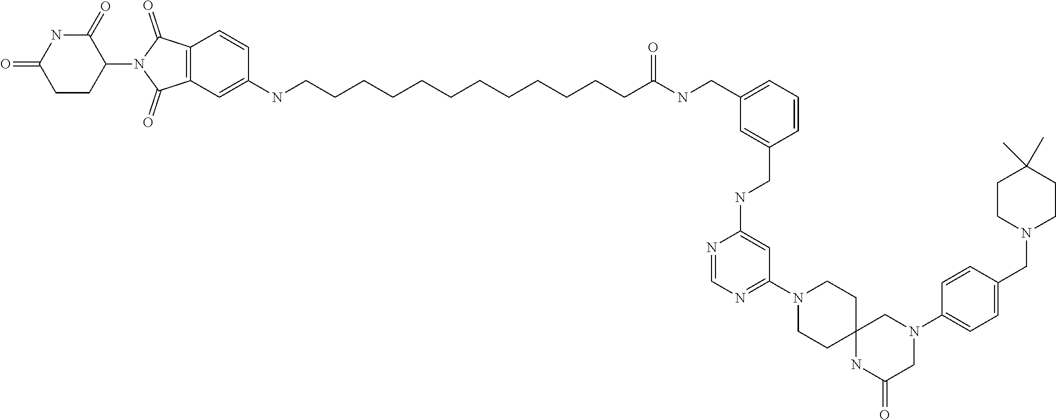 [SMILES: CC1(C)CCN(Cc2ccc(N3CC(=O)NC4(CCN(c5cc(NCc6cccc(CNC(=O)CCCCCCCCCCCCNc7ccc8c(c7)C(=O)N(C7CCC(=O)NC7=O)C8=O)c6)ncn5)CC4)C3)cc2)CC1] |
| 347 | 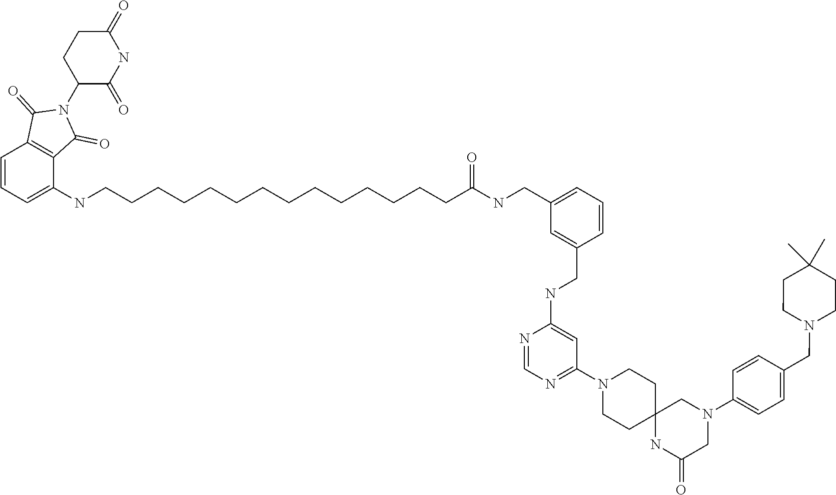 [SMILES: CC1(C)CCN(Cc2ccc(N3CC(=O)NC4(CCN(c5cc(NCc6cccc(CNC(=O)CCCCCCCCCCCCCCNc7cccc8c7C(=O)N(C7CCC(=O)NC7=O)C8=O)c6)ncn5)CC4)C3)cc2)CC1] |
| 348 | 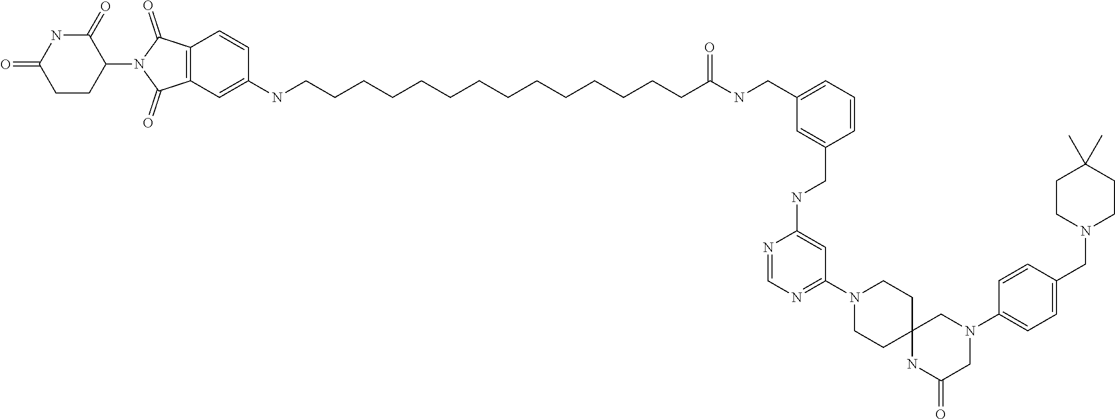 [SMILES: CC1(C)CCN(Cc2ccc(N3CC(=O)NC4(CCN(c5cc(NCc6cccc(CNC(=O)CCCCCCCCCCCCCCNc7ccc8c(c7)C(=O)N(C7CCC(=O)NC7=O)C8=O)c6)ncn5)CC4)C3)cc2)CC1] |
| 349 | 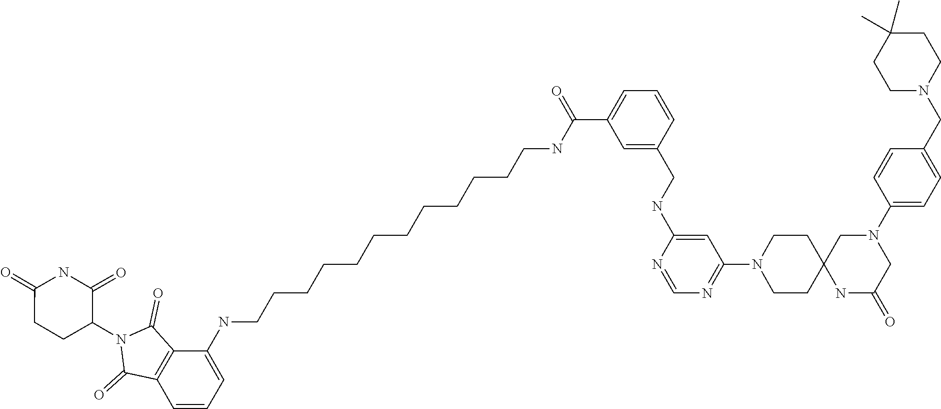 [SMILES: CC1(C)CCN(Cc2ccc(N3CC(=O)NC4(CCN(c5cc(NCc6cccc(C(=O)NCCCCCCCCCCCCNc7cccc8c7C(=O)N(C7CCC(=O)NC7=O)C8=O)c6)ncn5)CC4)C3)cc2)CC1] |
| 350 | 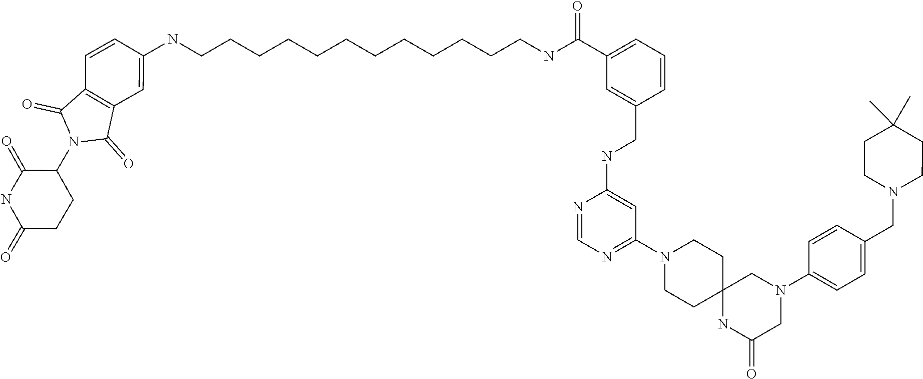 [SMILES: CC1(C)CCN(Cc2ccc(N3CC(=O)NC4(CCN(c5cc(NCc6cccc(C(=O)NCCCCCCCCCCCCNc7ccc8c(c7)C(=O)N(C7CCC(=O)NC7=O)C8=O)c6)ncn5)CC4)C3)cc2)CC1] |
| 351 | 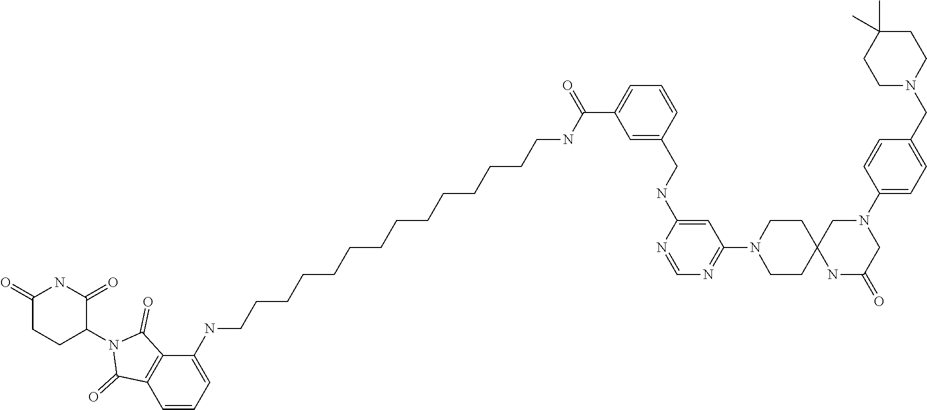 [SMILES: CC1(C)CCN(Cc2ccc(N3CC(=O)NC4(CCN(c5cc(NCc6cccc(C(=O)NCCCCCCCCCCCCCCNc7cccc8c7C(=O)N(C7CCC(=O)NC7=O)C8=O)c6)ncn5)CC4)C3)cc2)CC1] |
| 352 | 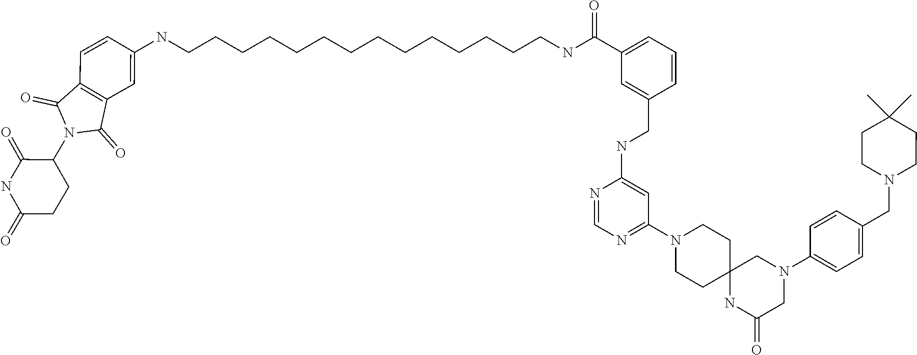 [SMILES: CC1(C)CCN(Cc2ccc(N3CC(=O)NC4(CCN(c5cc(NCc6cccc(C(=O)NCCCCCCCCCCCCCCNc7ccc8c(c7)C(=O)N(C7CCC(=O)NC7=O)C8=O)c6)ncn5)CC4)C3)cc2)CC1] |
| 353 | 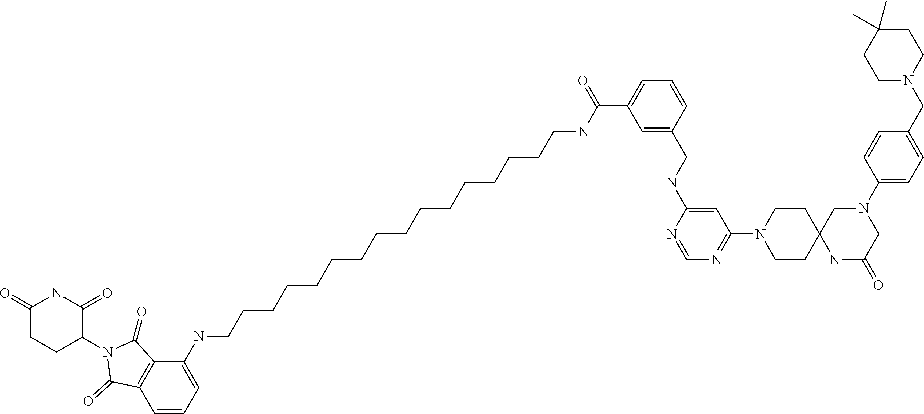 [SMILES: CC1(C)CCN(Cc2ccc(N3CC(=O)NC4(CCN(c5cc(NCc6cccc(C(=O)NCCCCCCCCCCCCCCCCNc7cccc8c7C(=O)N(C7CCC(=O)NC7=O)C8=O)c6)ncn5)CC4)C3)cc2)CC1] |
| 354 | 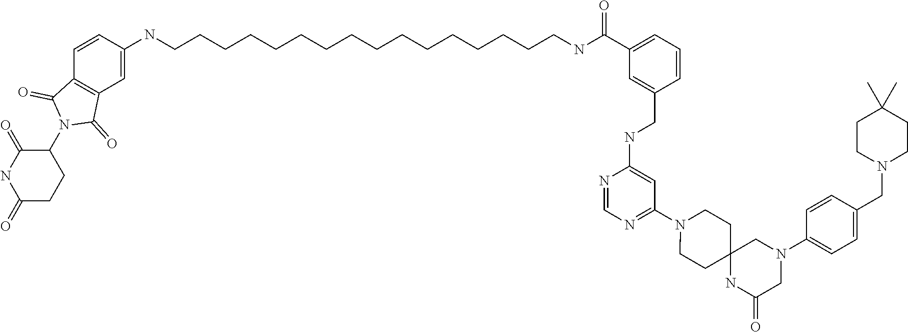 [SMILES: CC1(C)CCN(Cc2ccc(N3CC(=O)NC4(CCN(c5cc(NCc6cccc(C(=O)NCCCCCCCCCCCCCCCCNc7ccc8c(c7)C(=O)N(C7CCC(=O)NC7=O)C8=O)c6)ncn5)CC4)C3)cc2)CC1] |
| 355 | 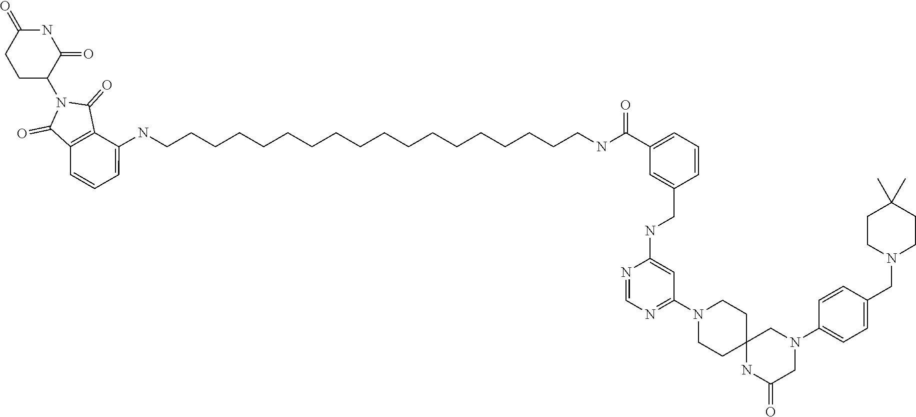 [SMILES: CC1(C)CCN(Cc2ccc(N3CC(=O)NC4(CCN(c5cc(NCc6cccc(C(=O)NCCCCCCCCCCCCCCCCCCNc7cccc8c7C(=O)N(C7CCC(=O)NC7=O)C8=O)c6)ncn5)CC4)C3)cc2)CC1] |
| 356 | 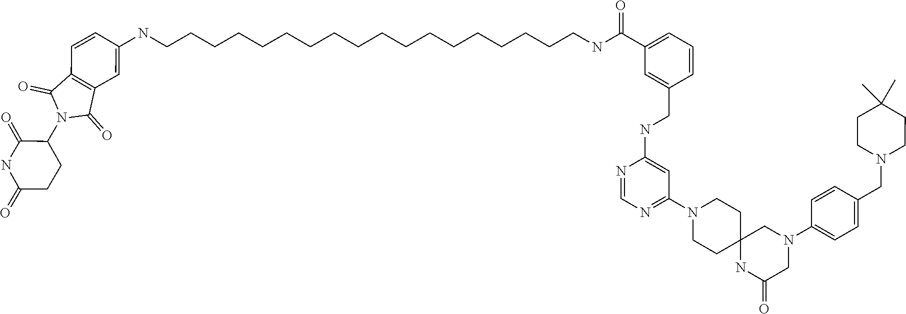 [SMILES: CC1(C)CCN(Cc2ccc(N3CC(=O)NC4(CCN(c5cc(NCc6cccc(C(=O)NCCCCCCCCCCCCCCCCCCNc7ccc8c(c7)C(=O)N(C7CCC(=O)NC7=O)C8=O)c6)ncn5)CC4)C3)cc2)CC1] |
| 357 | 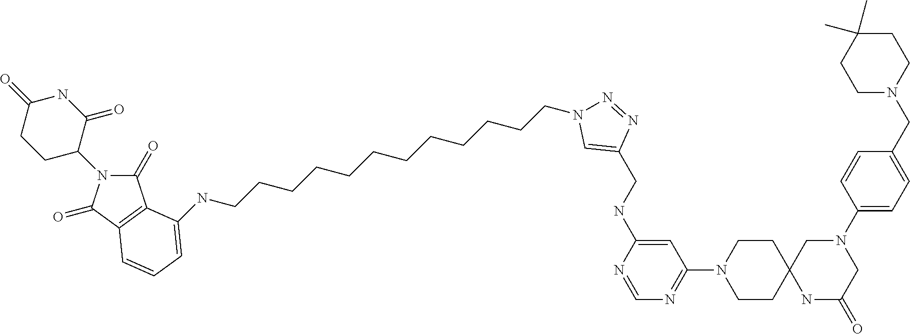 [SMILES: CC1(C)CCN(Cc2ccc(N3CC(=O)NC4(CCN(c5cc(NCc6cn(CCCCCCCCCCCCNc7cccc8c7C(=O)N(C7CCC(=O)NC7=O)C8=O)nn6)ncn5)CC4)C3)cc2)CC1] |
| 358 | 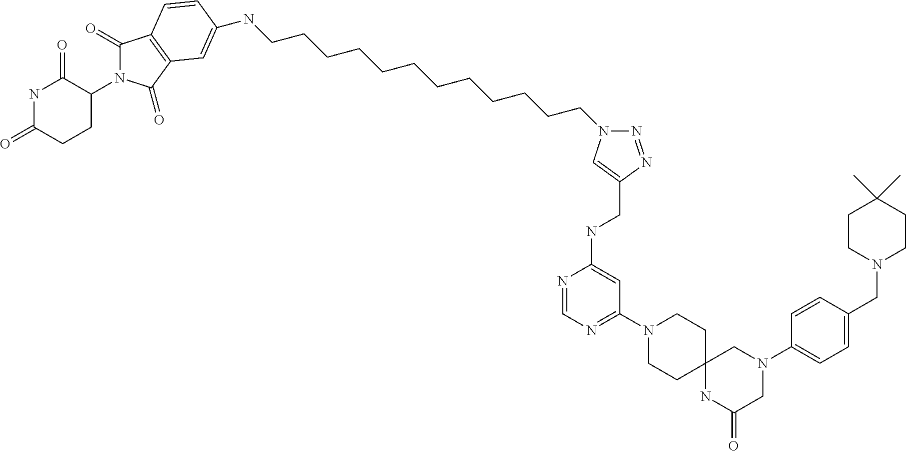 [SMILES: CC1(C)CCN(Cc2ccc(N3CC(=O)NC4(CCN(c5cc(NCc6cn(CCCCCCCCCCCCNc7ccc8c(c7)C(=O)N(C7CCC(=O)NC7=O)C8=O)nn6)ncn5)CC4)C3)cc2)CC1] |
| 359 | 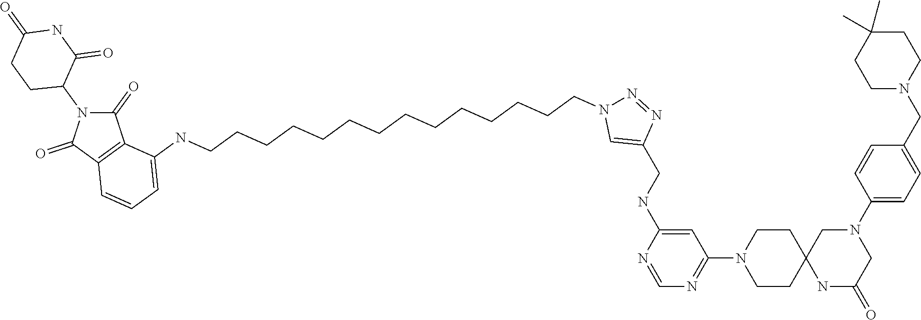 [SMILES: CC1(C)CCN(Cc2ccc(N3CC(=O)NC4(CCN(c5cc(NCc6cn(CCCCCCCCCCCCCCNc7cccc8c7C(=O)N(C7CCC(=O)NC7=O)C8=O)nn6)ncn5)CC4)C3)cc2)CC1] |
| 360 | 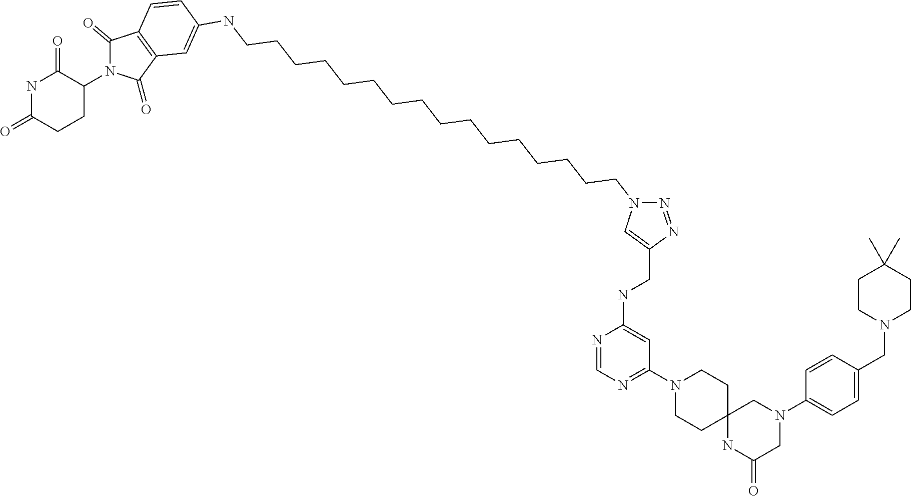 [SMILES: CC1(C)CCN(Cc2ccc(N3CC(=O)NC4(CCN(c5cc(NCc6cn(CCCCCCCCCCCCCCCCNc7ccc8c(c7)C(=O)N(C7CCC(=O)NC7=O)C8=O)nn6)ncn5)CC4)C3)cc2)CC1] |
| 361 | 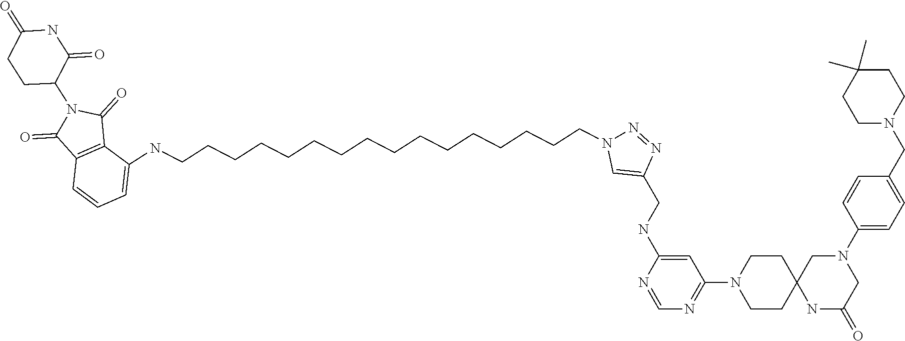 [SMILES: CC1(C)CCN(Cc2ccc(N3CC(=O)NC4(CCN(c5cc(NCc6cn(CCCCCCCCCCCCCCCCNc7cccc8c7C(=O)N(C7CCC(=O)NC7=O)C8=O)nn6)ncn5)CC4)C3)cc2)CC1] |
| 362 | 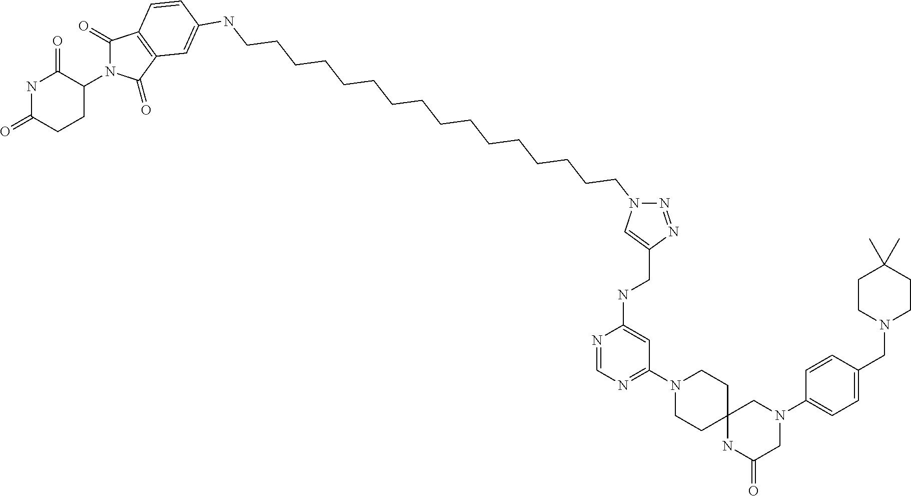 [SMILES: CC1(C)CCN(Cc2ccc(N3CC(=O)NC4(CCN(c5cc(NCc6cn(CCCCCCCCCCCCCCCCNc7ccc8c(c7)C(=O)N(C7CCC(=O)NC7=O)C8=O)nn6)ncn5)CC4)C3)cc2)CC1] |
| 363 | 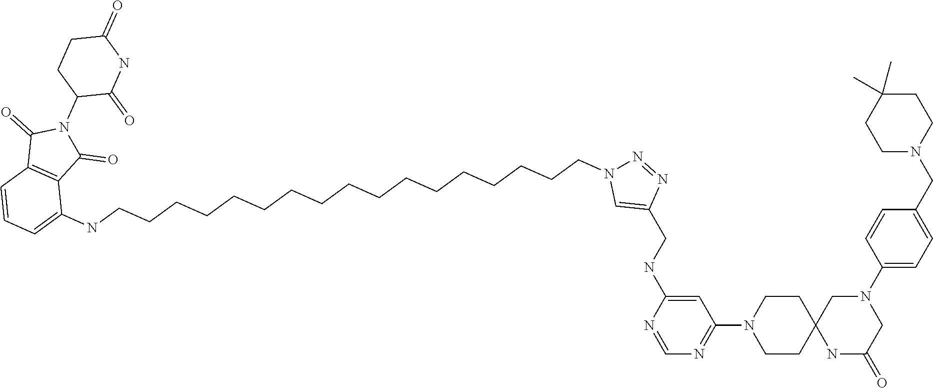 [SMILES: CC1(C)CCN(Cc2ccc(N3CC(=O)NC4(CCN(c5cc(NCc6cn(CCCCCCCCCCCCCCCCCNc7cccc8c7C(=O)N(C7CCC(=O)NC7=O)C8=O)nn6)ncn5)CC4)C3)cc2)CC1] |
| 364 | 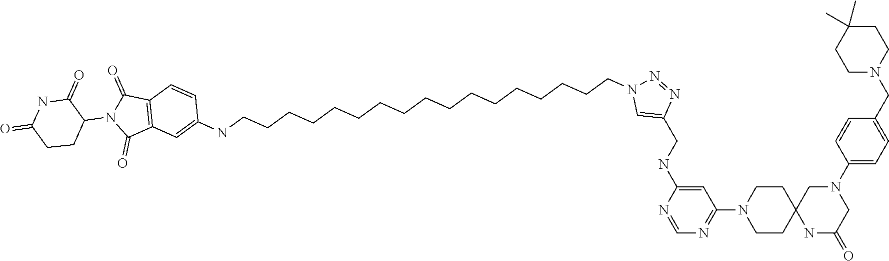 [SMILES: CC1(C)CCN(Cc2ccc(N3CC(=O)NC4(CCN(c5cc(NCc6cn(CCCCCCCCCCCCCCCCCNc7ccc8c(c7)C(=O)N(C7CCC(=O)NC7=O)C8=O)nn6)ncn5)CC4)C3)cc2)CC1] |

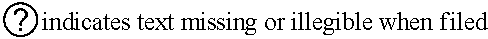

### Active Compound of the PROTAC

A second aspect of the disclosure relates to a compound of the general formula (I)

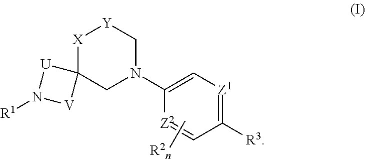

An alternative of the second aspect of the disclosure relates to a compound of the general formula (Ia)

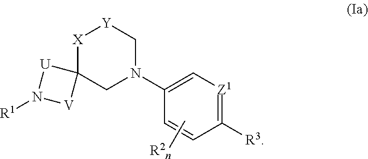

An alternative of the second aspect of the disclosure relates to a compound of the general formula (Ib)

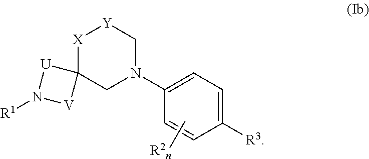

wherein
   Z¹ and Z² are independently selected from N, CH and CR²;
   X is O or NH;
   Y is CH₂, C═O, or SO₂;
   R¹ is an unsubstituted or substituted moiety selected from aryl, heteroaryl, cycloalkyl, and a heterocycle, particularly R¹ is unsubstituted or substituted heteroaryl;
   R² is selected from F, Me, Cl, OH, NH₂, Br, CF₃, CHF₂, CH₂F;
   n is an integer selected from 0, 1, 2, 3, and 4;
   R³ is a substituted alkylamine;
   U and V are independently selected from —CH₂— and —(CH₂)₂—, or one of U and V is —CH₂— and the other one is —(CH₂)₃—.

In certain embodiments, X is NH. In certain embodiments, Y is C═O. In certain embodiments, n is an integer selected from 0, 1, and 2. In certain embodiments, U and V are both —CH₂— or are both —(CH₂)₂—.

In certain embodiments, the compound is of the general formula (U)

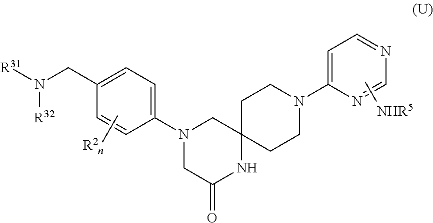

NR³¹R³² is selected from

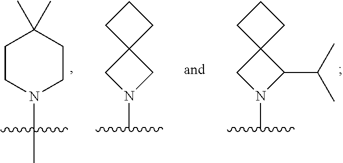

R² is selected from the group comprising F, Cl, CF₃, CHF₂, CH₂F;
n is an integer selected from 0, 1, 2, 3, and 4;
R⁵ is selected from an alkyl, an alkylaryl, a heteroalkylaryl, a cycloalkyl, an aryl, a heteroaryl and a heterocycle.

In certain embodiments, R² is F. In certain embodiments, n is an integer selected from 0, 1, and 2. In certain embodiments, n is 2. In certain embodiments, R⁵ is selected from an alkyl, an alkylaryl, and a cycloalkyl. In certain embodiments, R⁵ is selected from methyl and methylphenyl.

### Central Spirocycle

In certain embodiments, X is NH. In certain embodiments, Y is C═O.

In certain embodiments, the moiety

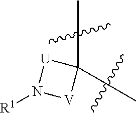

is selected from

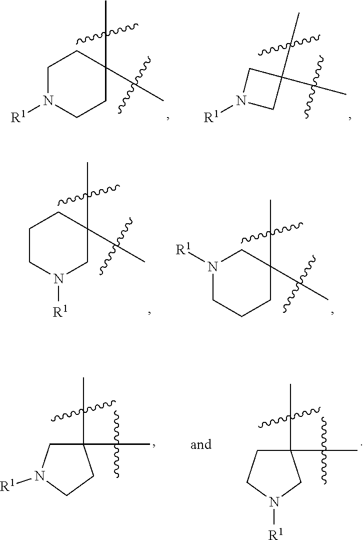

In certain embodiments, the moiety

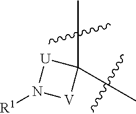

is selected from

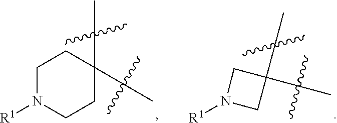

### Moiety R¹

In certain embodiments, R¹ is unsubstituted or substituted heteroaryl. In certain embodiments, R¹ is unsubstituted or substituted with a moiety selected from
a secondary amine NHR^{N}, wherein R^{N} is selected from a C₁-C₆ alkyl, a C₄-C₆ cycloalkyl, an aryl, and a heteroaryl, an alkylaryl, and an alkylheteroaryl;
a halogen, particularly Cl or F;
a C₁-C₆ alkyl, a C₄-C₆ cycloalkyl, an aryl, and a heteroaryl.

In certain embodiments, R¹ is unsubstituted or substituted with a moiety selected from
a secondary amine NHR^{N}, wherein R^{N} is selected from a C₁-C₆ alkyl, a C₄-C₆ cycloalkyl, an aryl, and a heteroaryl;
a halogen, particularly Cl or F.

In certain embodiments, the compound is of the general formula (II)

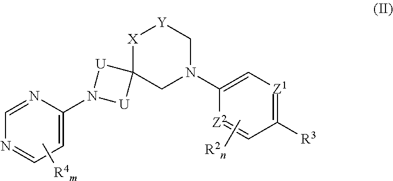

wherein
   Z¹, Z², X, Y, R², R³, U, V, and n have the same definitions as defined above;
   each R⁴ is independently selected from
      a secondary amine substituted with an alkyl, an alkylaryl, a heteroalkylaryl, a cycloalkyl, an aryl, a heteroaryl and/or a heterocycle,
      a halogen;
   and/or two R⁴ together form an unsubstituted or substituted heteroaryl or heterocycle;
m is an integer selected from 0, 1, 2, and 3.

In certain embodiments, the compound is of the general formula (III)

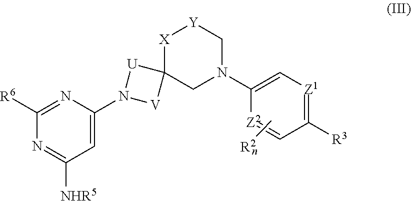

wherein
Z¹, Z², X, Y, R², R³, U, V, and n have the same definitions as defined above;
R⁵ is selected from an alkyl, an alkylaryl, a heteroalkylaryl, a cycloalkyl, an aryl, a heteroaryl and a heterocycle;
R⁶ is selected from halogen and hydrogen.

In certain embodiments, the compound is of the general formula (IV)

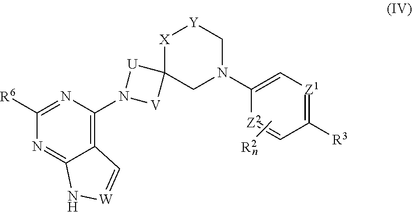

wherein
Z¹, Z², X, Y, R², R³, U, V, and n have the same definitions as defined above;
R⁶ is selected from halogen and hydrogen;
W is selected from N and CH.

In certain embodiments, at least one of Z¹ and Z² is CH or CR². In certain embodiments, both Z¹ and Z² are CH or CR².

### Moiety R³

In certain embodiments, R³ is substituted with one or several moieties selected independently from alkyl-, hydroxy-, amino-, amine-, halogen-, cycloalkyl-, and heterocycle- moieties.

In certain embodiments, R³ is substituted C₁-C₄ alkylamine. In certain embodiments, R³ is substituted C₁-C₂ alkylamine.

In certain embodiments, R³ is

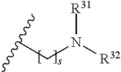

wherein
   s is an integer selected from 1 and 2, more particularly s is 1;
   R³¹ and R³² together form a heterocycle or heterobicycle, which is unsubstituted or alkyl-, halogen-, and/or hydroxyl-substituted, or
   R³¹ and R³² are independently selected from hydrogen and unsubstituted or hydroxy-, and/or halogen-substituted alkyl or cycloalkyl.

In certain embodiments, R³¹ and/or R³² are unsubstituted or substituted with alkyl-, hydroxy-, halogen-, cycloalkyl-, heterocycle- and/or -groups. In certain embodiments, R³¹ and/or R³² are independently selected from H and unsubstituted or hydroxy-, and/or halogen-substituted alkyl and cycloalkyl. In certain embodiments, R³¹ and R³² together form a heterocycle or heterobicycle, which is unsubstituted or alkyl-, hydroxy-, and/or halogen-substituted.

In certain embodiments, NR³¹R³² is selected from

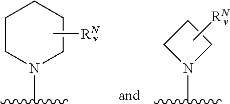

with v being an integer selected from 0, 1 and 2 and each R^{N} being independently selected from hydroxyl, halogen, and C₁-C₄ alkyl, or two R^{N} form a C₃-C₆ cycloalkyl.

In certain embodiments, —NR³¹R³² is selected from

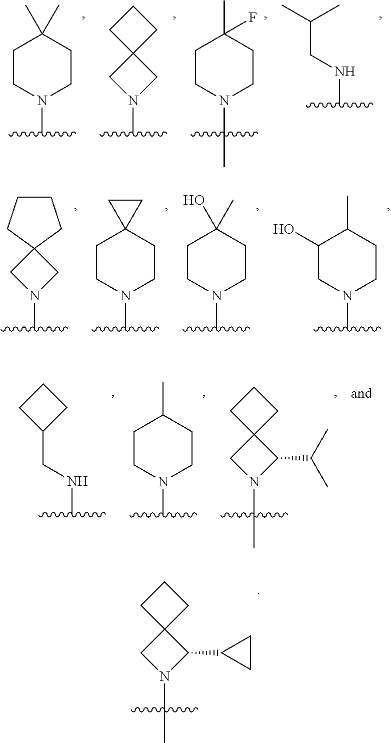

In certain embodiments, NR³¹R³² is selected from

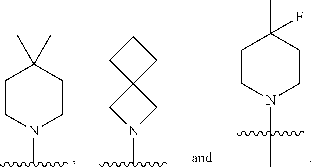

In certain embodiments, NR³¹R³² is

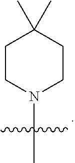 [SMILES: *N1CCC(C)(C)CC1]

In certain embodiments, NR³¹R³² is

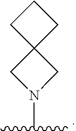 [SMILES: *N1CC2(CCC2)C1]

In certain embodiments, NR³¹R³² is

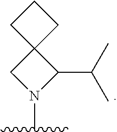 [SMILES: *N1CC2(CCC2)C1C(C)C]

In certain embodiments, NR³¹R³² is

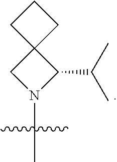 [SMILES: *N1CC2(CCC2)[C@@H]1C(C)C]

### Moiety R²

In certain embodiments, n is an integer selected from 0, 1, and 2. In certain embodiments, n is 2. In certain embodiments, R² is selected from F, Cl and OH. In certain embodiments, R² is F. R² can be bound to any of the carbon atoms of the aryl- or heteroaryl-ring. Thus, it can also be bound to Z¹ or Z² if they are carbon atoms.

### Moiety R⁵

In certain embodiments, R⁵ is selected from an alkyl, an alkylaryl, and a cycloalkyl. In certain embodiments, R⁵ is selected from methyl and methylphenyl.

### Use of the Compound

A third aspect of the disclosure relates to a compound according to the first or second aspect for use as a medicament.

A fourth aspect of the disclosure relates to a compound according to the first or second aspect for use in treatment of cancer.

In certain embodiments, the cancer is selected from the group comprising renal cancer, breast cancer, acute myeloid leukemia, hepatocellular carcinoma, and lung adenocarcinoma.

Similarly, within the scope of the present disclosure is a method or treating cancer in a patient in need thereof, comprising administering to the patient a compound according to the above description.

Similarly, a dosage form for the prevention or treatment of cancer is provided, comprising a non-agonist ligand or antisense molecule according to any of the above aspects or embodiments of the disclosure.

The skilled person is aware that any specifically mentioned drug may be present as a pharmaceutically acceptable salt of said drug. Pharmaceutically acceptable salts comprise the ionized drug and an oppositely charged counterion. Non-limiting examples of pharmaceutically acceptable anionic salt forms include acetate, benzoate, besylate, bitatrate, bromide, carbonate, chloride, citrate, edetate, edisylate, embonate, estolate, fumarate, gluceptate, gluconate, hydrobromide, hydrochloride, iodide, lactate, lactobionate, malate, maleate, mandelate, mesylate, methyl bromide, methyl sulfate, mucate, napsylate, nitrate, pamoate, phosphate, diphosphate, salicylate, disalicylate, stearate, succinate, sulfate, tartrate, tosylate, triethiodide and valerate. Non-limiting examples of pharmaceutically acceptable cationic salt forms include aluminium, benzathine, calcium, ethylene diamine, lysine, magnesium, meglumine, potassium, procaine, sodium, tromethamine and zinc.

Unless otherwise stated, compounds described herein may exhibit their natural isotopic abundance, or one or more of the atoms may be artificially enriched in a particular isotope having the same atomic number, but an atomic mass or mass number different from the atomic mass or mass number predominantly found in nature. All isotopic variations of the compounds of the present disclosure, whether radioactive or not, are encompassed within the scope of the present disclosure.

For example, the compounds described herein may be artificially enriched in one or more particular isotopes. In some embodiments, the compounds described herein may be artificially enriched in one or more isotopes that are not predominantly found in nature. In some embodiments, the compounds described herein may be artificially enriched in one or more isotopes selected from deuterium (²H), tritium (³H), iodine-125 (¹²⁵I) or carbon-14 (¹⁴C). In some embodiments, the compounds described herein are artificially enriched in one or more isotopes selected from ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, ¹³¹I, and ¹²⁵I. In some embodiments, the abundance of the enriched isotopes is independently at least 1%, at least 10%, at least 20%, at least 30%, at least 40%, at least 50%, at least 60%, at least 70%, at least 80%, at least 90%, or 100% by molar. In some embodiments, the compound is deuterated in at least one position. In some embodiments, the compounds disclosed herein have some or all of the ¹H atoms replaced with ²H atoms.

In some embodiments, the compounds described herein exist as geometric isomers. In some embodiments, the compounds described herein possess one or more double bonds. The compounds presented herein include all cis, trans, syn, anti, entgegen (E), and zusammen (Z) isomers as well as the corresponding mixtures thereof. In some situations, the compounds described herein possess one or more chiral centers and each center exists in the R configuration, or S configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as their individual stereoisomers by reacting a racemic mixture of the compound with an optically active resolving agent to form a pair of diastereoisomeric compounds, separating the diastereomers and recovering the optically pure enantiomers. In some embodiments, dissociable complexes are preferred. In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent, by any practical means that would not result in racemization.

In some situations, compounds exist as tautomers. The compounds described herein include all possible tautomers within the formulas described herein. Tautomers are compounds that are interconvertible by migration of a hydrogen atom, accompanied by a switch of a single bond and adjacent double bond. In bonding arrangements where tautomerization is possible, a chemical equilibrium of the tautomers will exist. All tautomeric forms of the compounds disclosed herein are contemplated. The exact ratio of the tautomers depends on several factors, including temperature, solvent, and pH.

Dosage forms may be for enteral administration, such as nasal, buccal, rectal, transdermal or oral administration, or as an inhalation form or suppository. Alternatively, parenteral administration may be used, such as subcutaneous, intravenous, intrahepatic or intramuscular injection forms. Optionally, a pharmaceutically acceptable carrier and/or excipient may be present.

### Pharmaceutical Composition and Administration

Another aspect of the disclosure relates to a pharmaceutical composition comprising a compound of the present disclosure, or a pharmaceutically acceptable salt thereof, and a pharmaceutically acceptable carrier. In further embodiments, the composition comprises at least two pharmaceutically acceptable carriers, such as those described herein.

In certain embodiments of the disclosure, the compound of the present disclosure is typically formulated into pharmaceutical dosage forms to provide an easily controllable dosage of the drug and to give the patient an elegant and easily handleable product.

The pharmaceutical composition can be formulated for oral administration, parenteral administration, or rectal administration. In addition, the pharmaceutical compositions of the present disclosure can be made up in a solid form (including without limitation capsules, tablets, pills, granules, powders or suppositories), or in a liquid form (including without limitation solutions, suspensions or emulsions).

The dosage regimen for the compounds of the present disclosure will vary depending upon known factors, such as the pharmacodynamic characteristics of the particular agent and its mode and route of administration; the species, age, sex, health, medical condition, and weight of the recipient; the nature and extent of the symptoms; the kind of concurrent treatment; the frequency of treatment; the route of administration, the renal and hepatic function of the patient, and the effect desired. In certain embodiments, the compounds of the disclosure may be administered in a single daily dose, or the total daily dosage may be administered in divided doses of two, three, or four times daily.

In certain embodiments, the pharmaceutical composition or combination of the present disclosure can be in unit dosage of about 1-1000 mg of active ingredient(s) for a subject of about 50-70 kg. The therapeutically effective dosage of a compound, the pharmaceutical composition, or the combinations thereof, is dependent on the species of the subject, the body weight, age and individual condition, the disorder or disease or the severity thereof being treated. A physician, clinician or veterinarian of ordinary skill can readily determine the effective amount of each of the active ingredients necessary to prevent, treat or inhibit the progress of the disorder or disease.

The pharmaceutical compositions of the present disclosure can be subjected to conventional pharmaceutical operations such as sterilization and/or can contain conventional inert diluents, lubricating agents, or buffering agents, as well as adjuvants, such as preservatives, stabilizers, wetting agents, emulsifiers and buffers, etc. They may be produced by standard processes, for instance by conventional mixing, granulating, dissolving or lyophilizing processes. Many such procedures and methods for preparing pharmaceutical compositions are known in the art, see for example L. Lachman et al. The Theory and Practice of Industrial Pharmacy, 4th Ed, 2013 (ISBN 8123922892).

### Method of Manufacture and Method of Treatment

The disclosure further encompasses, as an additional aspect, the use of a compound as identified herein, or its pharmaceutically acceptable salt, as specified in detail above, for use in a method of manufacture of a medicament for the treatment or prevention of cancer.

Similarly, the disclosure encompasses methods of treatment of a patient having been diagnosed with a disease associated with cancer. This method entails administering to the patient an effective amount of a compound as identified herein, or its pharmaceutically acceptable salt, as specified in detail herein.

Wherever alternatives for single separable features such as, for example, a ligand type or medical indication, are laid out herein as “embodiments”, it is to be understood that such alternatives may be combined freely to form discrete embodiments of the disclosure disclosed herein. Thus, any of the alternative embodiments for a ligand type may be combined with any medical indication mentioned herein.

The application further encompasses the following items:

### Items

1. A compound of the general formula (I)

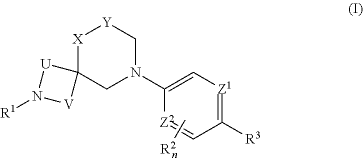

Z¹ and Z² are independently selected from N, CH and CR²;
X is O or NH, particularly X is NH;
Y is CH₂, C═O, or SO₂, particularly Y is C═O;
R¹ is an unsubstituted or substituted moiety selected from aryl, heteroaryl, cycloalkyl, and a heterocycle, particularly R¹ is unsubstituted or substituted heteroaryl;
R² is selected from F, Me, Cl, OH, NH₂, Br, CF₃, CHF₂, CH₂F;
n is an integer selected from 0, 1, 2, 3, and 4, particularly n is an integer selected from 0, 1, and 2;
R³ is a substituted alkylamine;
U and V are independently selected from —CH₂— and —(CH₂)₂—, or one of U and V is —CH₂— and the other one is —(CH₂)₃—,
particularly U and V are both —CH₂— or are both —(CH₂)₂—.
2. The compound according to item 1, wherein
   R¹ is unsubstituted or substituted with a moiety selected from
      a secondary amine NRN, wherein R^{N} is selected from a C₁-C₆ alkyl, a C₄-C₆ cycloalkyl, an aryl, and a heteroaryl, an alkylaryl, and an alkylheteroaryl;
      a halogen, particularly Cl or F; and
      a C₁-C₆ alkyl, a C₄-C₆ cycloalkyl, an aryl, and a heteroaryl.
3. The compound according to any one of the preceding items, wherein
   R³ is substituted with one or several moieties selected independently from alkyl-, hydroxy-, amino-, amine-, halogen-, cycloalkyl-, and heterocycle-moieties.
4. The compound according to any one of the preceding items, wherein R³ is C₁-C₄ alkylamine, particularly R³ is C₁-C₂ alkylamine.
5. The compound according to any one of the preceding items, wherein the compound is of the general formula (II)

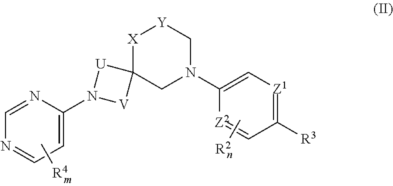

wherein
   Z¹, Z², X, Y, R², R³, U, V, and n have the same definitions as in item 1;
   each R⁴ is independently selected from
      a secondary amine substituted with an alkyl, an alkylaryl, a heteroalkylaryl, a cycloalkyl, an aryl, a heteroaryl and/or a heterocycle, particularly substituted with an alkyl, an alkylaryl, or a cycloalkyl;
      a halogen;
   and/or two R⁴ together form an unsubstituted or substituted heteroaryl or heterocycle;
   m is an integer selected from 0, 1, 2, and 3.
6. The compound according to any one of the preceding items, wherein the compound is of the general formula (III)

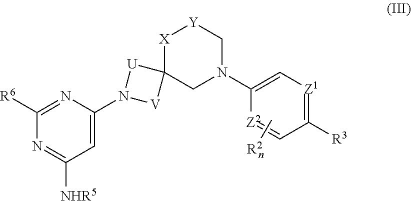

wherein
   Z¹, Z², X, Y, R², R³, U, V, and n have the same definitions as in item 1;
   R⁵ is selected from an alkyl, an alkylaryl, a heteroalkylaryl, a cycloalkyl, an aryl, a heteroaryl and a heterocycle,
   particularly R⁵ is selected from an alkyl, an alkylaryl, and a cycloalkyl;
   R⁶ is selected from halogen and hydrogen.
7. The compound according to any one of the preceding items 1 to 4, wherein the compound is of the general formula (IV)

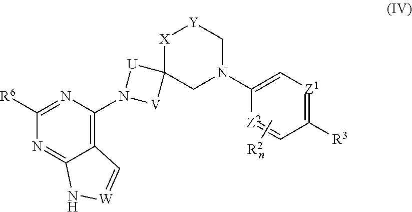

wherein
   Z¹, Z², X, Y, R², R³, U, V, and n have the same definitions as in item 1;
   R⁶ is selected from halogen and hydrogen;
   W is selected from N and CH.
8. The compound according to any one of the preceding items, wherein at least one of Z¹ and Z² is CH or CR², particularly both Z¹ and Z² are CH or CR².
9. The compound according to any one of the preceding items, wherein R³ is

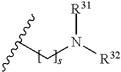

wherein
   s is an integer selected from 1 and 2, more particularly s is 1;
   R³¹ and R³² together form a heterocycle or heterobicycle, which is unsubstituted or alkyl-, halogen-, and/or hydroxyl-substituted, or
   R³¹ and R³² are independently selected from hydrogen and unsubstituted or hydroxy-, and/or halogen-substituted alkyl or cycloalkyl,
   particularly R³¹ and R³² together form a heterocycle or heterobicycle, which is unsubstituted or alkyl-, halogen-, and/or hydroxyl-substituted.
10. The compound according to item 9, wherein NR³¹R³² is selected from

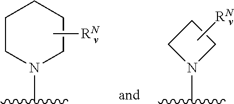

with v being an integer selected from 0, 1 and 2 and each R^{N} being independently selected from hydroxyl, halogen, and C₁-C₄ alkyl, or two R^{N} form a C₃-C₆ cycloalkyl.
11. The compound according to any one of the preceding items 9 to 10, wherein NR³¹R³² is selected from

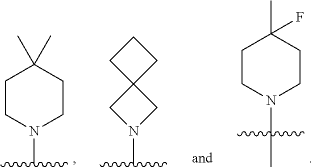

12. The compound according to any one of the preceding items, wherein R² is selected from F, Cl and OH, particularly R² is F.
13. A compound according to any of the preceding items for use as a medicament.
14. A compound according to any of the preceding items 1 to 12 for use in treatment of cancer.

The disclosure is further illustrated by the following examples and figures, from which further embodiments and advantages can be drawn. These examples are meant to illustrate the disclosure but not to limit its scope.

### EXAMPLES

All reagents were purchased from commercial suppliers and used as received. Reactions run at elevated temperature were carried out in the oil bath. All reactions were monitored by thin-layer chromatography (Aluminium plates coated with silica gel 60 F₂₅₄). Flash column chromatography was carried out over silica gel (0.040-0.063 mm). ¹H and ¹³C {¹H} NMR spectra were recorded on AV2-400 MHz and AV600 Bruker spectrometers (400 MHz, 101 MHz and 600 MHz, 150 MHz, respectively) in DMSO or CDCl₃ Chemical shifts are given in ppm and their calibration was performed to the residual ¹H and ¹³C signals of the deuterated solvents. Multiplicities are abbreviated as follows: singlet (s), doublet (d), multiplet (m), and broad signal (bs). The purity was acquired by Liquid chromatography high resolution electrospray ionization mass spectrometry (LC-HR-ESI-MS): Acquity UPLC (Waters, Milford, USA) connected to an Acquity eλ diode array detector and a Synapt G2 HR-ESI-QTOF-MS (Waters, Milford, USA); injection of 1 μL sample (c=ca. 10-100 g/mL in the indicated solvent); Acquity BEH C18 HPLC column (1.7 m particle size, 2×50 mm, Waters) kept at 30° C.; * elution at a flow rate of 400 L/min with A: H₂O+1% HCO₂H and B: CH₃CN+0.1% HCO₂H, linear gradient from 5-98% B within 5 min, then isocratic for 1 min; *UV spectra recorded from 200-600 nm at 1.2 nm resolution and 20 points s⁻¹; ESI: positive ionization mode, capillary voltage 3.0 kV, sampling cone 40V, extraction cone 4V, N₂ cone gas 4 L/h, N₂ desolvation gas 800 L/min, source temperature 120° C.; mass analyzer in resolution mode: mass range 100-2′000 m/z with a scan rate of 1 Hz; mass calibration to <2 ppm within 50-2′500 m/z with a 5 mM aq. soln. of HCO₂Na, lockmasses: m/z 195.0882 (caffein, 0.7 ng/mL) and 556.2771 (Leucine-enkephalin, 2 ng/mL).

### General Procedure for Buchwald-Hartwig Coupling:

To a stirred solution of the corresponding halide (1 equiv.) in dioxane (0.3 M), under a nitrogen atmosphere, the corresponding amine (1 equiv.) was added. Nitrogen gas was bubbled through the reaction for two minutes and Cs₂CO₃ (1.2 equiv), Ruphos Pd G4 (10 mol %) and Ruphos (10 mol %) were added. The reaction mixture was stirred at 150° C. for 17 h, concentrated under reduced pressure and the obtained residue was purified by flash column chromatography.

### General Procedure for Boc Group Deprotection:

To a stirred solution of the corresponding Boc protected amine in MeOH (0.3 M), HCl (0.9 M, 37% aq.) was added. The reaction mixture was stirred at 25° C. for 4 h and the reaction mixture was concentrated under reduced pressure. The obtained residue was directly engaged in the next step without further purification.

General Procedure for S_{N}Ar with 4,6-dichloropyrimidine

To a stirred solution of the corresponding amine (1 eq.) or amine hydrochloride salt (1 equiv.) in iPrOH (0.3 M), 4,6-dichloro-pyrimidine (1.2 equiv.) and Et₃N (1-4 equiv.) were added. The reaction mixture was stirred at 80° C. for 3 h in the microwave and concentrated under reduced pressure. The crude residue was dissolved in nBuOH, washed three times with water, once with brine, dried over MgSO₄ and concentrated under reduced pressure. The crude residue was coevaporated with toluene several times to remove the residual nBuOH and then purified by flash column chromatography.

General Procedure for S_{N}Ar with Chloropyrimidine Derivatives:

The corresponding chloropyrimidine (1 eq.) was dissolved in methylamine (0.1 M, 8 M in EtOH) or benzylamine (0.3 M) and the reaction mixture was stirred at 130° C. for 3 h (MeNH₂) or 140° C. for 8 h (BnNH₂) in the microwave. The crude residue was concentrated under reduced pressure and purified by flash column chromatography. For reactions with benzylamine, the crude residue was coevaporated with water then toluene several times to remove the benzylamine before performing the purification.

### Example 1

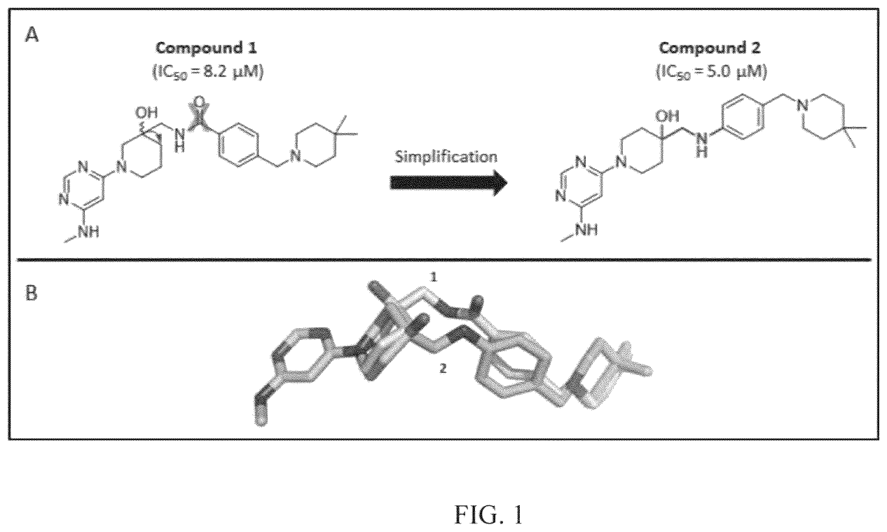

The inventors' design started at the roots of one of the inventor's early inhibitor (1, Table 4), with the aim to simplify the structure and reduce molecular weight (FIG. 1A). For this purpose, changing the methylene position from 1,3 to 1,4 on the piperidine ring removes the chiral center. In addition, according to the X-ray structure of 1 with METTL3, the amide C═O group deletion would allow to keep the original vector (FIG. 1B). These two modifications led to 2 and its two pyridine containing derivatives 3 and 4, which exhibited not only similar potency compared to the parent molecule (IC₅₀=5.0, 4.6, and 5.8 M respectively, Table 4), but also no chirality and a reduced heavy atom count, hence higher ligand efficiency (LE=0.23, 0.23, and 0.22, respectively). Because 4 had better lipophilic ligand efficiency (LLE=3.4, calculated with DataWarrior), its pyridine core was conserved in the next optimization stage. According to the crystal structure of the complex of METTL3 with inhibitor 1, methylamine to benzylamine replacement on the pyrimidine ring seemed beneficial to inhibition. This proved to be true as the corresponding derivative 5 showed a 6-fold increase in potency (IC₅₀=0.79 μM).

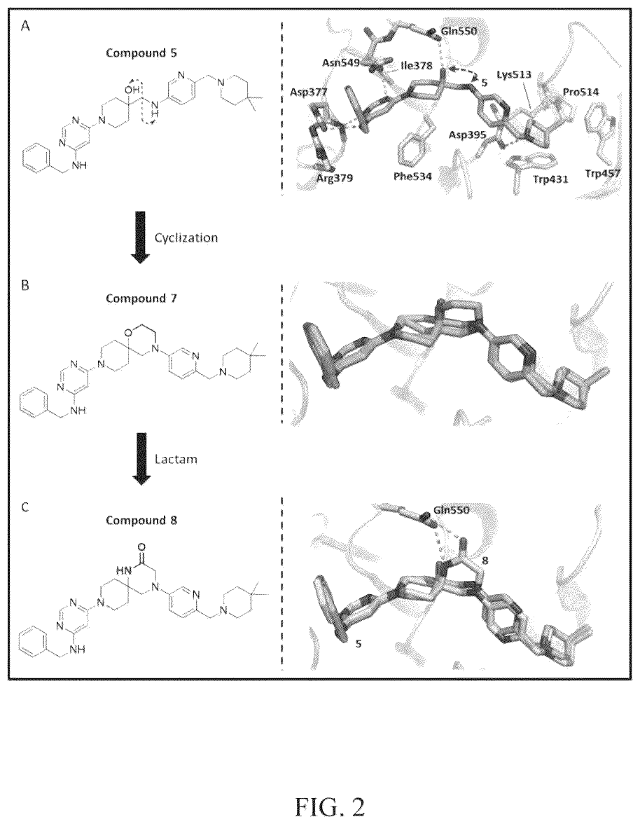

One striking feature of this inhibitor series is their linear shape coupled with sp³ acyclic atom linkers that makes them highly flexible. Rigidifying the structure is a viable way to freeze a ligand in its preferred conformation, which in turn can enhance the binding energy by reducing entropic penalties. Thus, the inventors envisioned two different strategies to achieve this goal: either making an amide connection between the piperidine and the pyridine ring, or according to compound 5's conformation, a spirocycle could be formed by connecting the tertiary alcohol with the aniline (FIG. 2A). The two methods brought opposite results, the amide derivative 6 lost the previous potency boost (IC₅₀=3.6 μM, Table 4) while spirocycle 7 was promising both in terms of inhibition (IC₅₀=0.28 μM, Table 5) and novelty. The inventors managed to soak both 5 and 7 with METTL3, and the X-ray analysis showed a strong structural overlap. The pyrimidine moiety is engaged in two hydrogen bonds with NH backbone from Asn549 and Ile378 while involved in π-stacking with Phe534 and π interactions with Asn549 side chain (FIG. 2A). The benzylamine group interacts with Asp377 side chain and also forms a cation-π interaction with Arg379. On the opposite site of the binding pocket, the gem dimethyl group fills a lipophilic pocket formed by Lys513, Pro514, Trp457 and Trp431 residues, whereas the charged piperidine forms a salt-bridge with Asp395. The sole difference between 5 and 7 is, for the latter, the missing hydrogen bond between the tertiary alcohol and Gln550 side chain due to the alcohol transformation into an ether (FIG. 2B). The inventors envisaged that replacing the ether by a lactam could restore this interaction and even make an additional hydrogen bond thanks to the C═O group of the ligand and the NH₂ amide of Gln550. The inventors obtained a strong potency boost for the corresponding derivative 8 (IC₅₀=0.037 μM), and the inventor's hypothesis was confirmed by the two hydrogen bond interactions found in the crystal structure (FIG. 2C). Furthermore, both LE and LLE improved substantially (0.25 and 4.4 respectively, Table 5).

ADME properties, such as solubility, cell permeability, and metabolic stability are essential for chemical probes, so they were considered early on in the project. The inventor's newly synthesized inhibitors (5, 7-8) displayed mixed results; however, all of them displayed mediocre stability towards enzymatic degradation with half-lives lower than 12 minutes upon incubations with rat liver microsomes (Tables 4, 5). Therefore, the inventors focused on improving ADME properties while getting better biochemical potency. The initial approach was to substitute the pyridine nitrogen atom by a carbon atom, yielding 9 with moderate permeability (9·10⁻⁶ cm·s⁻¹) and, surprisingly, slightly increased solubility (Table 5). However, metabolic stability remained unchanged, so the benzylamine was replaced with methylamine (10). Indeed, solubility and metabolic stability were significantly improved (108 μM and 107 min, respectively) as well as LE and LLE values (0.28 and 4.5, respectively), but at the expense of limited permeability (2·10⁻⁶ cm·s⁻¹) and a 3.4-fold potency reduction. From 10, two other possibilities of decreasing the size of the inventor's molecules were pursued: replacing spiropiperidine with spiroazetidine (11) and spirolactam with spirourea (12). Unfortunately, both displayed a substantial loss in potency (5 and 20 fold, respectively). Yet, the spiroazetidine moiety remains a potential alternative helping to reduce molecular weight and to improve physicochemical properties at a later stage. Next, the inventor's strategy was oriented towards permeability improvement. Lactam methylation in compound 13 resulted in a serious decrease in potency (19 fold), demonstrating the crucial role of the lactam hydrogen bond interactions. After thorough spiro scaffold optimization, the inventors turned their attention to the pyrimidine motif. Addition of one more methyl on the aniline (14) was highly detrimental to binding compared to 10 (0.97 and 0.089 μM, respectively, Table 6), probably due to loss of the hydrogen bond to the side chain of Asp377, while methyl to isopropyl substitution (15) showed a less pronounced reduction as the hydrogen bond is preserved (0.33 μM). These two modifications illustrated the limited space available for branched sp³ carbons at this position. Surprisingly, substitution with a cyclopropyl group (16) was not only well tolerated (0.084 μM), but it also improved the three ADME properties (Table 6) and could become a promising alternative for lead optimization. S-Adenosyl methionine (SAM) is the natural ligand of METTL3 that contains an adenosine scaffold overlapping with the pyrimidine group of the inventor's inhibitors, thus the inventors thought to test a few bicyclic heteroaromatic modifications. The pyrrolopyrimydine 17 had a slight increase in potency in comparison to 10, but similarly low permeability and a larger efflux ratio in the Caco-2 assay (Table 6). The interaction geometry between the N³ pyrimidine atom and Asn549 nitrogen backbone seemed not optimal, thus the inventors thought to remove this pyrimidine nitrogen atom in order to improve permeability and possibly to suppress a partial desolvation penalty. The latter proved to be false since pyrrolopyridine 18 exhibited a severe binding loss (74 fold). Incorporation of a chlorine atom between the two pyrimidine nitrogen atoms (19) was beneficial for potency (0.024 μM); however, solubility and metabolic stability were critically impaired (45 μM and 32 min, respectively), which prompted the inventors to look for different modifications.

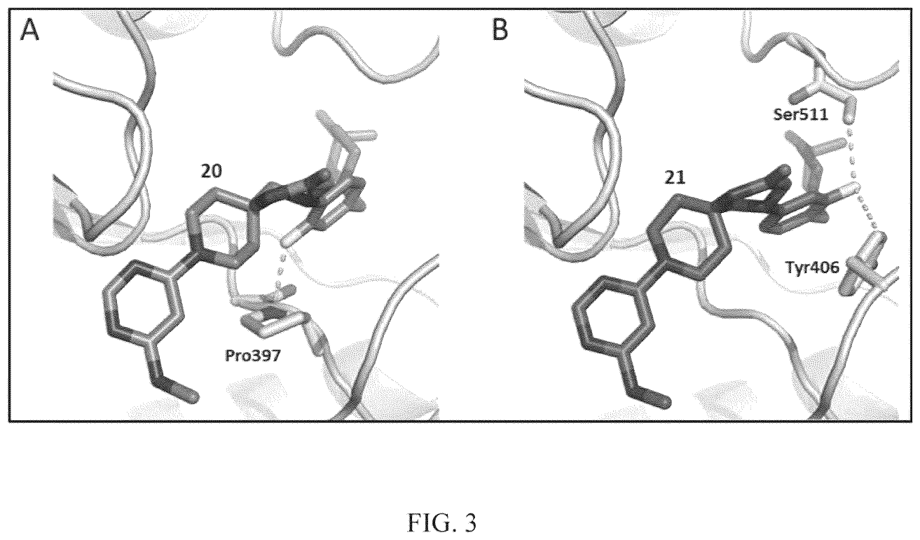

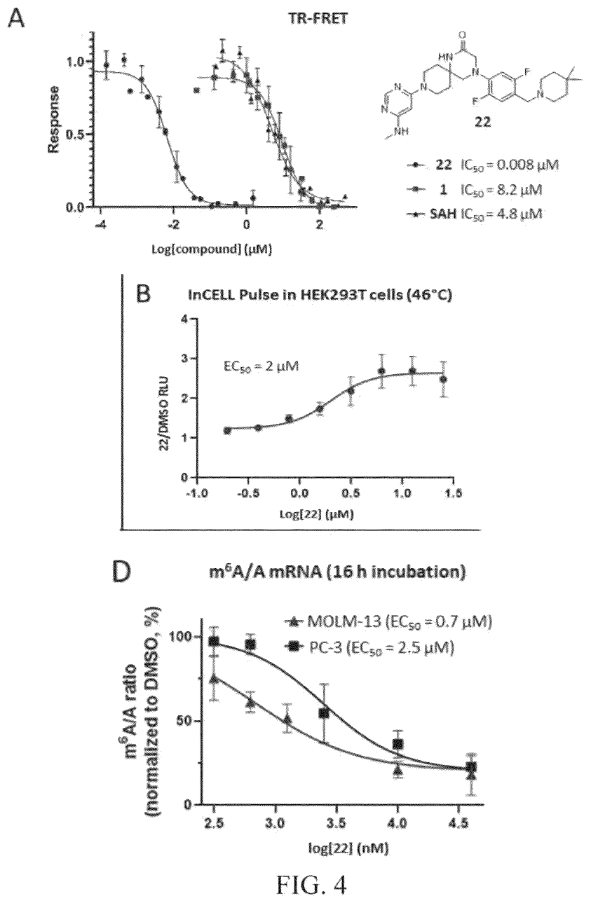

Because the spiro scaffold and the pyrimidine moiety were already optimized, the inventors considered the phenyl ring as the next target region. Several publications discuss the unique properties of fluorine atoms that can translate into unexpected and promising results in drug design. Indeed, fluorine atoms are able to make unusual interactions, and aromatic fluorine atoms tend to increase permeability. A fluorine scan was performed on the phenyl ring, affording two novel derivatives 20 and 21. Compared to the inhibitor 10, both compounds improved binding to a similar extent (0.038 and 0.032 μM, respectively); however, permeability was considerably increased only for 20 (Table 7). An X-ray structures in complex with METTL3 were solved for each molecule and revealed that the fluorine in 21 displays hydrophobic contacts (FIG. 3B), whereas the fluorine atom of 20 is also engaged in an unusual interaction with the nitrogen n system of Pro397 (FIG. 3A). Inhibitor 20 was preferable because of its strong improvement in permeability and small efflux ratio (9·10⁻⁶ cm·s⁻¹ and 2, respectively), but the combination of both fluorine atoms quickly emerged as the key solution to achieve excellent potency and to keep adequate ADME properties. Indeed, compound 22 exhibited single digit nanomolar IC₅₀ (0.008 μM) in the TR-FRET assay (Table 7 and FIG. 4A), high cell permeability (12·10⁻⁶ cm·s⁻¹), and favorable values of LE and LLE (0.3 and 5.3, respectively), as well as acceptable metabolic stability (t_{1/2}=24 min).

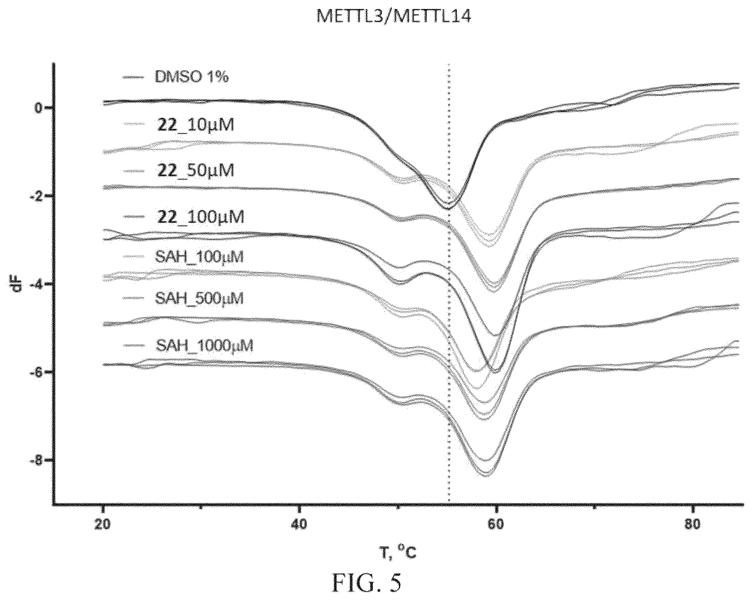

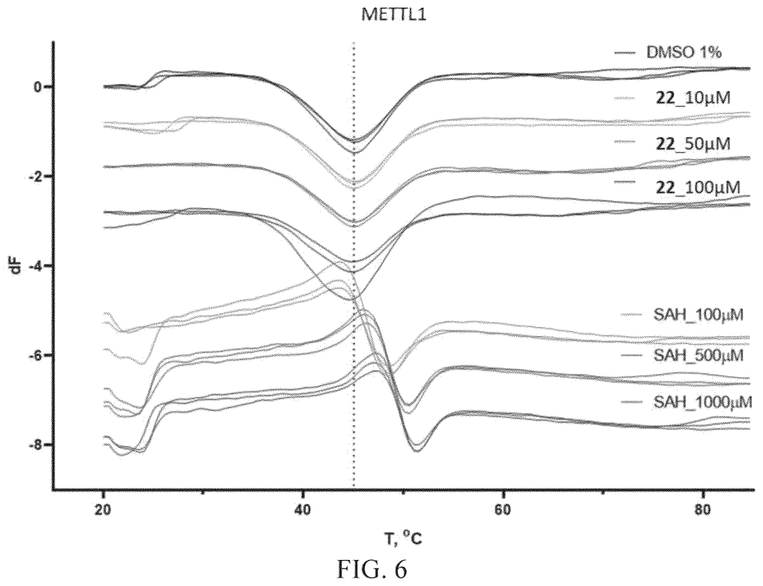

To investigate the selectivity of compound 22 towards other RNA methyltransferases, the inventors conducted protein thermal shift assay. The inventors expressed and purified METTL1 protein that is a writer of 7-methylguanosine mark on tRNA, mRNA, and miRNAs and serves as a representative closely related protein. The inventors employed as positive control S-adenosyl-L-homocysteine (SAH), a by-product of RNA methyltransferase catalytic activity and a natural binder, which showed ΔTₘ of 2.8° C. and 3.5° C. at 100 μM for METTL3/METTL14 and METTL1, respectively (FIGS. 5 and 6). Compound 22 at 100 μM was able to shift the melting temperature of METTL3/METTL14 by 4.7° C. compared to DMSO control (FIGS. 5 and 6). On the contrary, no shift was observed for METTL1 with compound 22 up to 100 μM indicating no binding.

The enhanced thermal stabilization of METTL3 by compound 22 allowed the inventors to study its cellular target engagement in two orthogonal assays based on protein thermal denaturation. The binding of 22 was evaluated in InCELL Pulse assay where enhanced ProLabel® (ePL) enzyme fragment fused to the N-terminus of the truncated METTL3 (residues 354-580) was expressed in HEK293T cells. After the incubation of these cells with inhibitor 22 for 1 h at 37° C., cells were heated at 46° C. for 3 min, and the non-aggregated METTL3-ePL protein was quantified using luminescence-based assay (FIG. 4B). Compound 22 stabilized the METTL3-ePL fusion protein with an EC₅₀ of 2 μM in HEK293T cells. Therefore, both experiments brought clear evidence of cell permeability and cellular target engagement. Finally, to highlight the biological potential of 22 as an inhibitor of METTL3 enzymatic activity, the inventors measured m⁶A/A ratio in polyadenylated RNA in two distinct cancer cell lines, MOLM-13 (AML) and PC-3 (prostate cancer) cells after 16 hours of compound treatment. The inventors found that 22 was able to reduce this ratio down to 10-20% of DMSO-treated control samples and with a certain degree of selectivity between the two cell lines (EC₅₀=0.7 and 2.5 μM for MOLM-13 and PC-3 respectively, FIG. 4D).

The inventors successfully improved potency (by a factor of 1 000), efficiency parameters, and ADME properties of a series of METTL3 inhibitors by protein crystallography-guided medicinal chemistry. The key features were rigidification thanks to the design of spiro scaffolds and the use of fluorine atoms at specific positions. The most potent inhibitor (compound 22) shows an IC₅₀ of 8 nM in a TR-FRET assay. No binding to the off-target METTL1 was observed at concentrations of up to 100 μM. Cellular target engagement of compound 22 was demonstrated using two different assays. Furthermore, for the reduction of m⁶A/A in polyadenylated RNA, as quantified by UPLC-MS/MS analysis, EC₅₀ values of 0.7 μM and 2.5 μM were measured in MOLM-13 (leukemia) and PC-3 (prostate cancer) cell lines. Thus, compound 22 is a chemical probe to decipher the functional role of METTL3/METTL14 and its involvement in hematological malignancies and solid tumors.

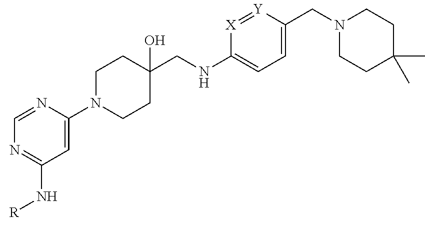

TABLE 4
Early modifications of the original scaffold.

| | | | | | | | | Kin- | P_{app A-B} | |
|---|---|---|---|---|---|---|---|---|---|---|
| | | | | | | | | etic | | |
| | | | | | | | | solu- | | |
| Nº | R | X | Y | IC₅₀₁ | MW² | LE³ | LLE⁴ | bility⁵ | (Efflux)⁶ | RLM⁷ |
| 1 | 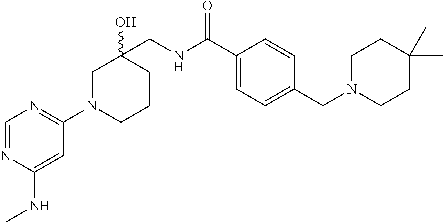 [SMILES: CNc1cc(N2CCCC(O)(CNC(=O)c3ccc(CN4CCC(C)(C)CC4)cc3)C2)ncn1] | | | 8.2 | 466.63 | 0.21 | 2.5 | — | — | — |
| 2 | Me | CH | CH | 5.0 | 438.62 | 0.23 | 2.5 | — | — | — |
| 3 | Me | N | CH | 4.6 | 439.61 | 0.23 | 3.2 | — | — | — |
| 4 | Me | CH | N | 5.8 | 439.61 | 0.22 | 3.4 | — | — | — |
| 5 | Bn | CH | N | 0.79 | 515.70 | 0.22 | 2.8 | 78 | 8 (5) | 5 |

| | | | | | | | | |
|---|---|---|---|---|---|---|---|---|
| 6 | 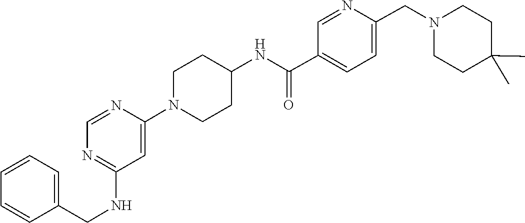 [SMILES: CC1(C)CCN(Cc2ccc(C(=O)NC3CCN(c4cc(NCc5ccccc5)ncn4)CC3)cn2)CC1] | 3.6 | 513.69 | 0.20 | 1.7 | — | — | — |

¹Time resolved-Förster resonance energy transfer (TR-FRET) assay (μM).
²g/mol.
³Ligand efficiency (kcal.mol⁻¹ heavy atom count⁻¹).
⁴Lipophilic ligand efficiency (pIC₅₀-logP);
⁵μM;
⁶10⁻⁶ cm·s⁻¹, (efflux ratio), Caco-2 experiment;
⁷Rat liver microsomes, t_{1/2} (min).

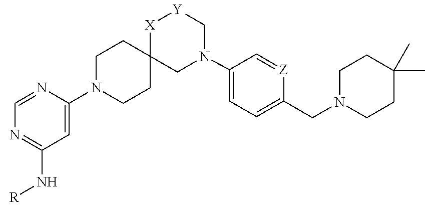

TABLE 5
Derivatization from the spiro scaffold.

| | | | | | | | | | Kinetic | P_{app A-B} | RLM |
|---|---|---|---|---|---|---|---|---|---|---|---|
| Nº | R | X | Y | Z | IC₅₀¹ | MW² | LE³ | LLE⁴ | solubility⁵ | (Efflux)⁶ | ⁷ |
| 7 | Bn | O | CH₂ | N | 0.28 | 541.74 | 0.22 | 2.8 | 61 | 15 (0.6) | 12 |
| 8 | Bn | NH | C═O | N | 0.037 | 554.74 | 0.25 | 4.4 | 44 | 2 (17) | 8 |
| 9 | Bn | NH | C═O | CH | 0.026 | 553.75 | 0.25 | 3.6 | 69 | 9 (3) | 9 |
| 10 | Me | NH | C═O | CH | 0.089 | 477.65 | 0.28 | 4.5 | 108 | 2 (9) | 107 |
| 11 | 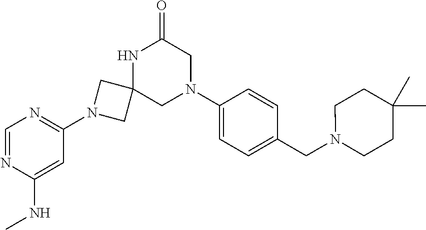 | | | | 0.44 | 449.60 | 0.26 | 4.5 | 86 | — | 70 |
| 12 | 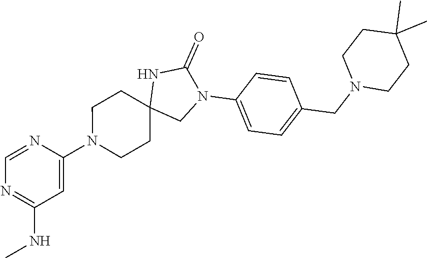 | | | | 1.8 | 463.63 | 0.23 | 2.6 | — | — | — |
| 13 | Me | NMe | C═O | CH | 1.5 | 491.68 | 0.22 | 3.0 | — | — | — |

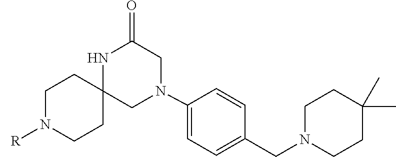

TABLE 6
Optimization of the aminopyrimidine ring.

| Compound | | | | | | Kinetic | P_{app A-B} | RLM |
|---|---|---|---|---|---|---|---|---|
| n° | R | IC₅₀¹ | MW² | LE³ | LLE⁴ | solubility⁵ | (Efflux)⁶ | ⁷ |
| 14 | 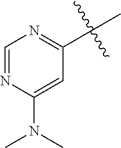 [SMILES: *c1cc(N(C)C)ncn1] | 0.97 | 491.68 | 0.23 | 3.2 | — | — | — |
| 15 | 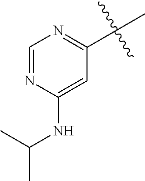 [SMILES: *c1cc(NC(C)C)ncn1] | 0.33 | 505.71 | 0.24 | 3.2 | — | — | — |
| 16 | 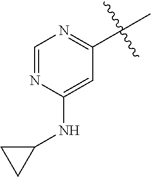 [SMILES: *c1cc(NC2CC2)ncn1] | 0.084 | 503.69 | 0.26 | 4.0 | 111 | 4 (7) | 118 |
| 17 | 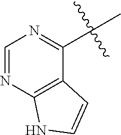 [SMILES: *c1ncnc2[nH]ccc12] | 0.061 | 487.65 | 0.27 | 4.3 | 73 | 2 (14) | 93 |
| 18 | 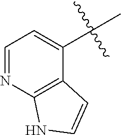 [SMILES: *c1ccnc2[nH]ccc12] | 4.5 | 486.66 | 0.20 | 2.3 | — | — | — |
| 19 | 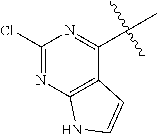 [SMILES: *c1nc(Cl)nc2[nH]ccc12] | 0.024 | 522.09 | 0.28 | 3.9 | 45 | — | 32 |

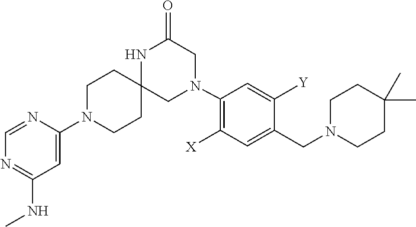

TABLE 7
Fluorine scan on the phenyl ring.

| Compound | | | | | | | Kinetic | P_{app A-B} | |
|---|---|---|---|---|---|---|---|---|---|
| no | X | Y | IC₅₀¹ | MW² | LE³ | LLE⁴ | solubility⁵ | (Efflux)⁶ | RLM⁷ |
| 20 | F | H | 0.038 | 495.64 | 0.28 | 4.8 | 72 | 9 (2) | 63 |
| 21 | H | F | 0.032 | 495.64 | 0.29 | 4.8 | 92 | 3 (8) | 46 |
| 22 | F | F | 0.008 | 513.64 | 0.30 | 5.3 | 54 | 12 (2) | 24 |

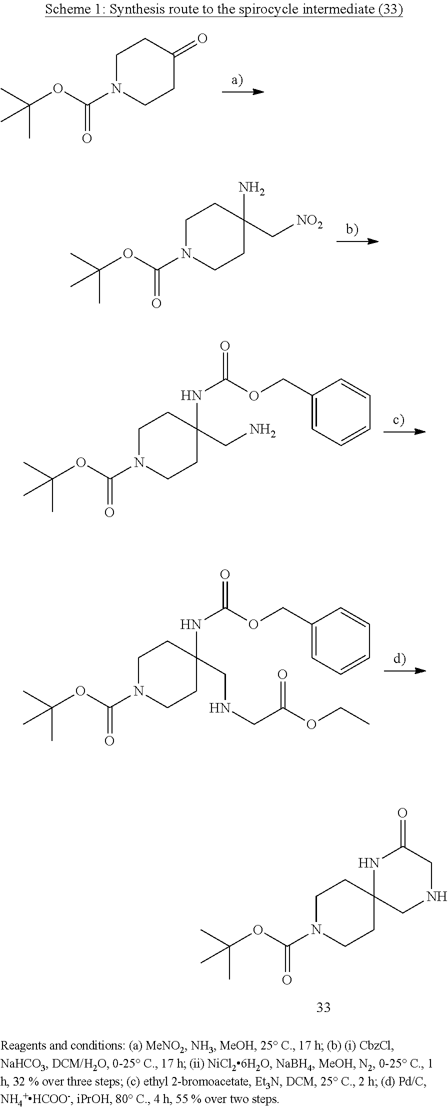

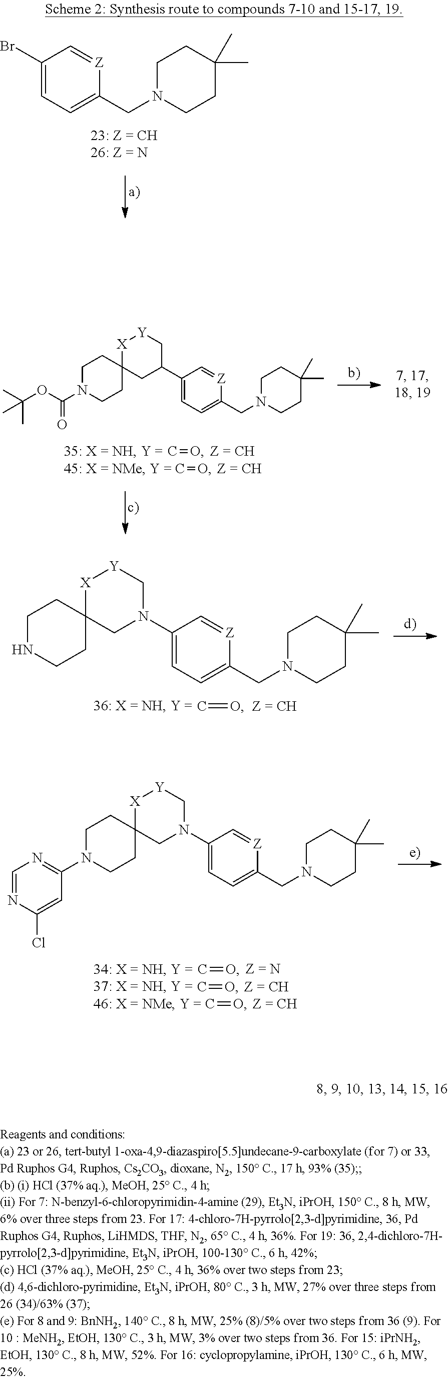

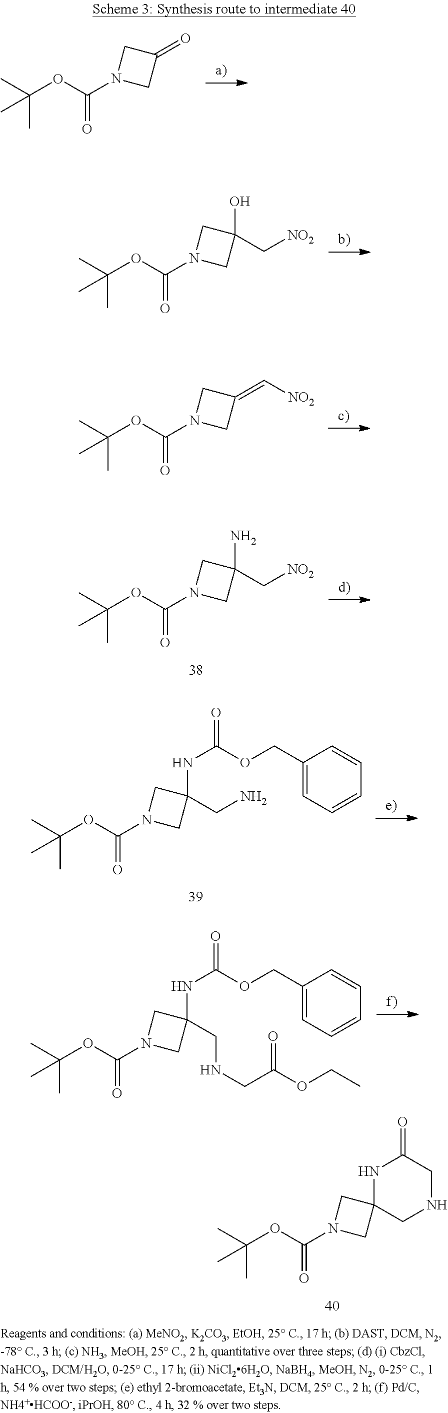

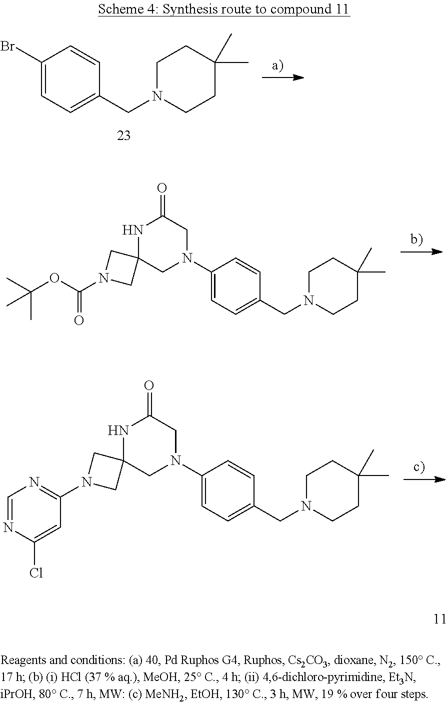

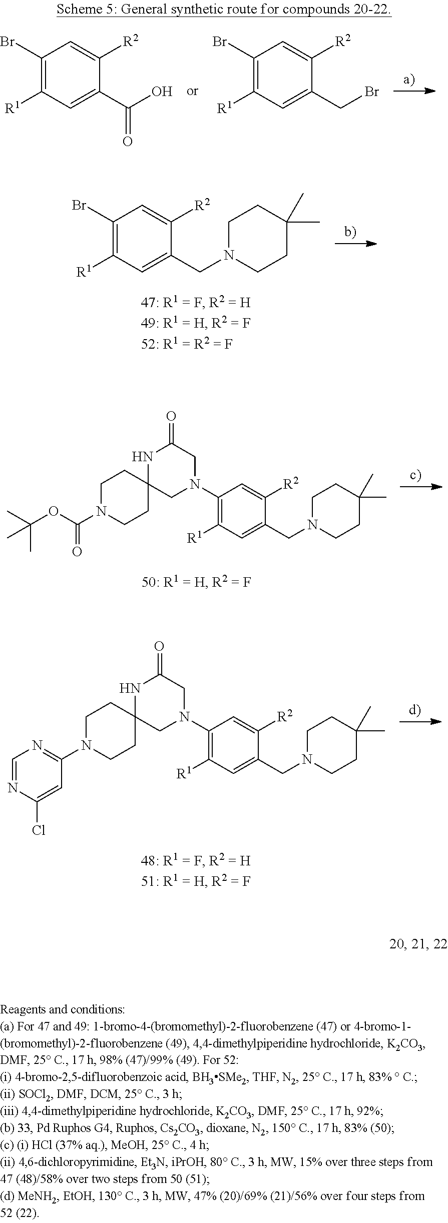

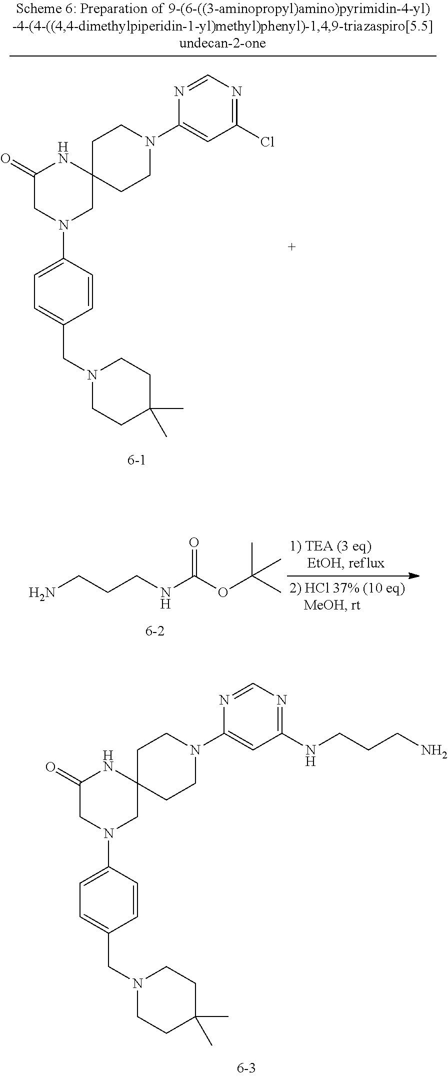 [SMILES: CC1(C)CCN(Cc2ccc(N3CC(=O)NC4(CCN(c5cc(Cl)ncn5)CC4)C3)cc2)CC1 ; CC(C)(C)OC(=O)NCCCN ; CC1(C)CCN(Cc2ccc(N3CC(=O)NC4(CCN(c5cc(NCCCN)ncn5)CC4)C3)cc2)CC1]

Chloropyrimidine derivative 6-1 (100 mg, 0.21 mmol) was dissolved in ethanol (0.5 M), to a stirred solution was subsequently added N-Boc-1,3-propanediamine 6-2 (108 mg, 0.62 mmol) and TEA (86.6 μL, 0.62 mmol). The resulting reaction mixture was stirred under reflux until completion (Monitored by TLC). The reaction mixture was evaporated and the crude product was purified using flash column chromatography (SiO₂; DCM/MeOH=9:1) and obtained as a slightly yellow solid (117 mg, 91%). The impure product of S_{N}Ar (117 mg, 0.19 mmol) was dissolved in MeOH (0.5 M) and HCl 37%(57.7 μL, 1.9 mmol) was added. The resulting reaction mixture was stirred at rt until completion (Monitored by TLC). The reaction mixture was evaporated, taken up with butanol, quenched with a saturated aqueous solution of Na₂CO₃ and extracted with butanol (3×10 ml). The combined organic layers were dried over MgSO₄, filtrated and evaporated to afford the product (80 mg, 81%).

¹H NMR (400 MHz, MeOD) δ 8.06 (d, J=0.9 Hz, 1H), 7.38 (d, J=8.2 Hz, 2H), 7.01 (d, J=8.5 Hz, 2H), 5.77 (d, J=1.0 Hz, 1H), 4.01 (s, 2H), 3.86 (s, 2H), 3.83 (s, 2H), 3.63-3.56 (m, 2H), 3.53 (s, 2H), 3.43 (t, J=6.5 Hz, 2H), 3.17 (d, J=0.8 Hz, 1H), 2.98 (t, J=7.0 Hz, 6H), 1.95-1.87 (m, 4H), 1.81 (ddd, J=13.3, 8.6, 4.1 Hz, 2H), 1.58 (d, J=5.9 Hz, 4H), 1.29 (d, J=2.7 Hz, 3H), 1.02 (s, 6H).

¹³C NMR (101 MHz, DMSO) δ 167.6, 163.7, 162.1, 157.8, 148.6, 130.1, 129.2, 114.7, 65.4, 62.4, 53.3, 52.9, 51.9, 49.7, 38.8, 38.0, 37.9, 34.8, 29.6, 28.8, 28.6, 15.7.

LRMS (ESI) m/z: [M+H]+ calcd for C29H44N8O; 520.36 found, 521.37.

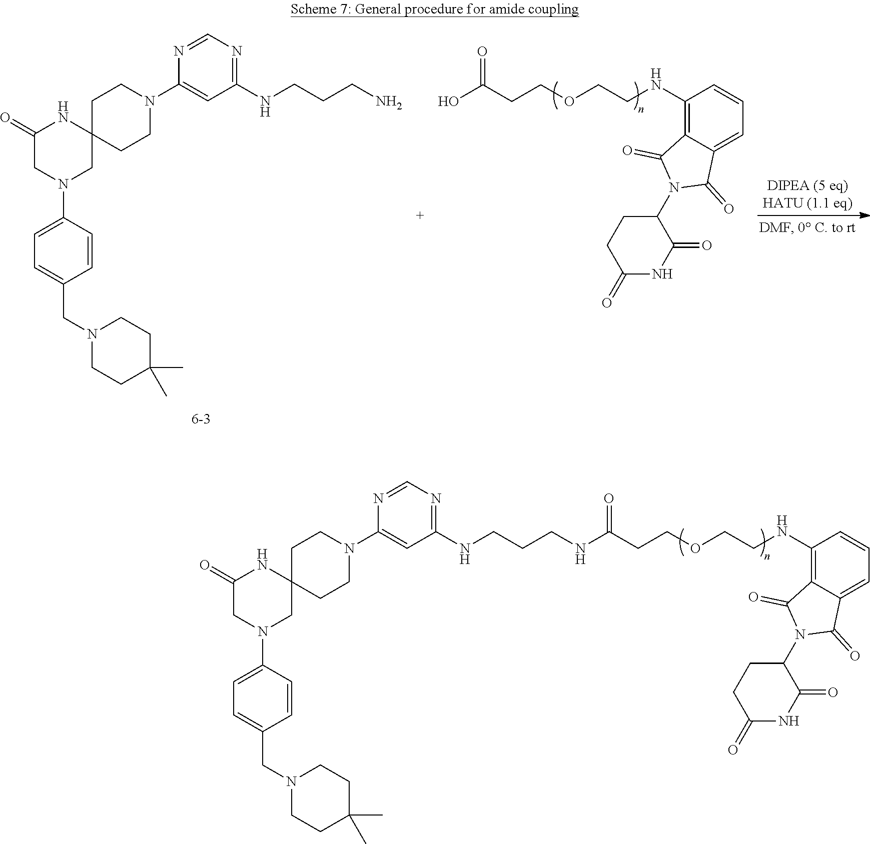

Carboxylic acid (1 equiv) was dissolved in DMF (0.5 M), the solution was cooled to 0° C. and DIPEA (5 equiv) was added. After 10 minutes HATU (1.1 equiv) was added and the solution was let stirring for 30 minutes after which amine 6-3 (1 equiv) was added. The resulting reaction mixture was stirred at rt until completion (Monitored by TLC).

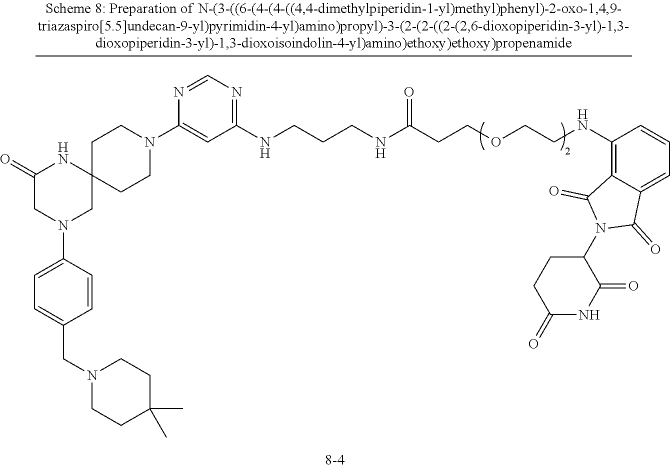

Compound 8-4 was prepared following the general amide coupling procedure 1 using amine 3 (159 mg, 0.306 mmol) and corresponding pomalidomide carboxylic acid (132 mg, 0.306 mmol). The crude product was purified first using flash column chromatography (Al₂O₃; DCM/MeOH=from 90:10 to 80:20), then 8-4 was obtained using semi preparative HPLC (15 mg, 5%).

¹H NMR (500 MHz, DMSO) δ 11.09 (s, 1H), 8.25 (s, 1H), 7.98 (s, 1H), 7.83 (t, J=5.6 Hz, 1H), 7.59-7.56 (m, 1H), 7.13-7.03 (m, 3H), 6.89 (d, J=8.4 Hz, 1H), 6.62-6.58 (m, 1H), 5.61 (s, 1H), 5.05 (dd, J=12.7, 5.4 Hz, 1H), 3.80-3.75 (m, 1H), 3.65 (s, 1H), 3.59 (t, J=6.0 Hz, 2H), 3.53-3.43 (m, 6H), 3.19-3.17 (m, 2H), 3.08 (q, J=6.6 Hz, 2H), 2.87 (ddd, J=16.7, 13.7, 5.4 Hz, 1H), 2.64-2.50 (m, 2H), 2.30-2.28 (m, 3H), 2.04-1.99 (m, 1H), 1.90 (s, 1H), 1.74-1.57 (m, 4H), 1.29-1.23 (m, 3H), 0.87 (s, 3H).

¹³C NMR (126 MHz, DMSO) δ 172.8, 172.1, 170.1, 170.1, 169.0, 167.3, 167.1, 163.3, 161.7, 157.4, 148.2, 146.4, 136.3, 132.1, 129.7, 128.6, 117.5, 114.3, 110.7, 109.3, 69.7, 69.6, 68.9, 66.9, 61.9, 52.8, 52.4, 51.4, 49.2, 48.6, 41.7, 38.3, 38.0, 36.3, 36.2, 34.3, 31.0, 29.1, 28.3, 22.2, 22.1, 21.1.

LRMS (ESI) m/z: [M+H]+ calcd for C49H65N1108; 935.50 found, 936.51.

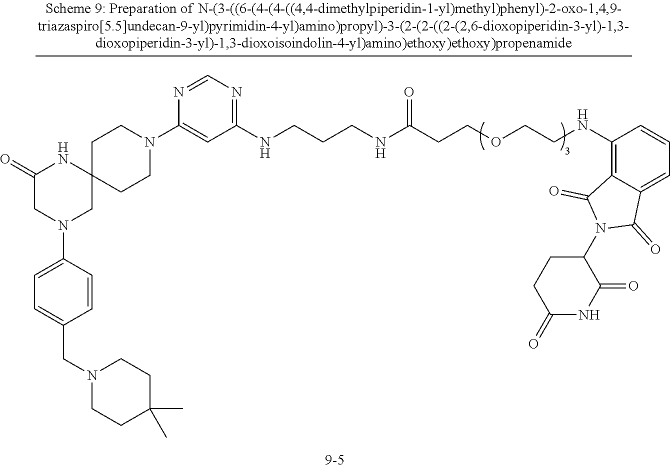

Compound 9-5 was prepared following the general amide coupling procedure 1 using amine 3 (69 mg, 0.13 mmol) and corresponding pomalidomide carboxylic acid (64 mg, 0.13 mmol). The crude product was purified first using flash column chromatography (Al₂O₃; DCM/MeOH=from 90:10 to 80:20), then 9-5 was obtained using semi preparative HPLC (28 mg, 22%).

¹H NMR (500 MHz, DMSO) δ 11.09 (s, 1H), 8.25 (s, 1H), 7.98 (s, 1H), 7.84 (t, J=5.6 Hz, 1H), 7.57 (t, J=7.8 Hz, 1H), 7.14-7.03 (m, 3H), 6.89 (d, J=8.2 Hz, 2H), 6.60 (m, 2H), 5.61 (s, 1H), 5.05 (dd, J=12.8, 5.4 Hz, 1H), 3.79-3.77 (m, 2H), 3.65 (s, 1H), 3.60-3.45 (m, 9H), 3.19-3.17 (m, 2H), 3.08 (q, J=6.6 Hz, 2H), 2.91-2.84 (m, 1H), 2.63-2.56 (m, 2H), 2.30-2.26 (m, 3H), 2.04-2.00 (m, 1H), 1.90 (s, 1H), 1.74-1.57 (m, 4H), 1.29-1.23 (m, 3H), 0.87 (s, 3H).

¹³C NMR (126 MHz, DMSO) δ 172.8, 170.1, 170.1, 169.0, 167.3, 167.1, 163.3, 161.7, 157.4, 148.1, 146.4, 136.3, 132.1, 129.7, 128.7, 117.5, 114.3, 110.7, 109.3, 69.8, 69.7, 69.6, 68.9, 66.9, 62.0, 52.8, 52.4, 51.4, 49.3, 48.6, 41.7, 38.4, 36.3, 36.2, 34.3, 31.0, 29.0, 28.3, 22.2, 21.2.

LRMS (ESI) m/z: [M+H]+ calcd for C51H69N1109; 979.53 found, 980.54.

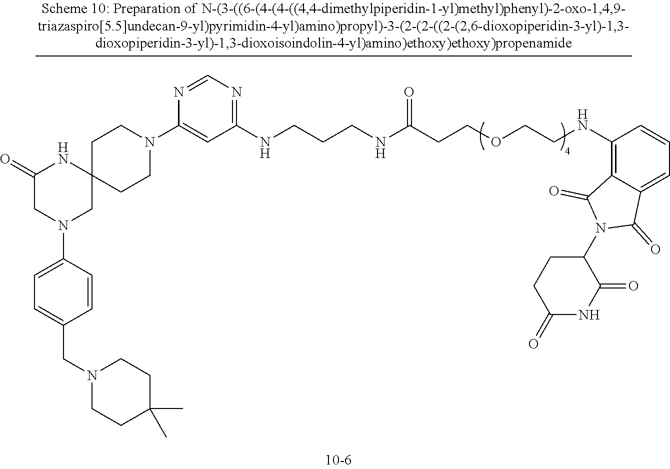

Compound 10-6 was prepared following the general amide coupling procedure 1 using amine 3 (50 mg, 0.096 mmol) and corresponding pomalidomide carboxylic acid (50 mg, 0.096 mmol). The crude product was purified first using flash column chromatography (Al₂O₃; DCM/MeOH=from 90:10 to 80:20), then 10-6 was obtained using semi preparative HPLC (20 mg, 20%).

¹H NMR (500 MHz, DMSO) δ 11.09 (s, 1H), 8.25 (s, 1H), 7.98 (s, 1H), 7.84 (t, J=5.7 Hz, 1H), 7.60-7.55 (m, 1H), 7.16-7.04 (m, 3H), 6.89 (d, J=8.4 Hz, 2H), 6.62-6.58 (m, 2H), 5.61 (s, 1H), 5.05 (dd, J=12.8, 5.5 Hz, 1H), 3.79-3.76 (m, 2H), 3.65 (s, 1H), 3.62-3.51 (m, 5H), 3.48-3.45 (m, 8H), 3.19-3.16 (m, 2H), 3.08 (q, J=6.5 Hz, 2H), 2.87 (ddd, J=16.8, 13.8, 5.4 Hz, 1H), 2.63-2.56 (m, 2H), 2.31-2.28 (m, 4H), 2.04-1.99 (m, 1H), 1.90 (s, 1H), 1.75-1.57 (m, 6H), 1.30-1.23 (m, 4H), 0.87 (s, 4H).

¹³C NMR (126 MHz, DMSO) δ 172.8, 172.1, 170.1, 170.1, 169.0, 167.3, 167.1, 163.3, 161.7, 157.4, 149.8, 148.2, 146.4, 136.3, 134.7, 132.1, 132.0, 129.8, 123.4, 122.1, 117.5, 114.3, 110.7, 109.3, 69.8, 69.8, 69.8, 69.7, 69.5, 68.9, 66.9, 61.9, 52.8, 52.4, 51.4, 49.2, 48.6, 41.7, 38.3, 38.0, 36.3, 36.2, 34.3, 31.0, 29.1, 28.3, 22.2.

LRMS (ESI) m/z: [M+H]+ calcd for C53H73N11010; 1023.55 found, 1024.56.

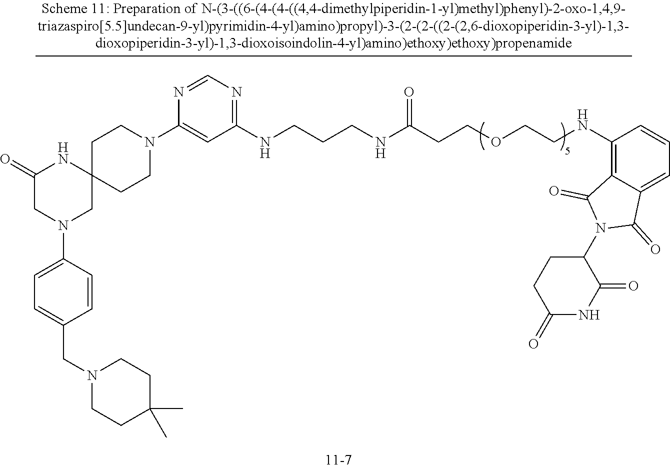

Compound 11-7 was prepared following the general amide coupling procedure 1 using amine 3 (51 mg, 0.098 mmol) and corresponding pomalidomide carboxylic acid (56 mg, 0.096 mmol). The crude product was purified first using flash column chromatography (Al₂O₃; DCM/MeOH=from 90:10 to 80:20), then 11-7 was obtained using semi preparative HPLC (13 mg, 12%).

¹H NMR (500 MHz, DMSO) δ 11.09 (s, 1H), 8.25 (s, 1H), 7.98 (s, 1H), 7.84 (t, J=5.7 Hz, 1H), 7.61-7.54 (m, 1H), 7.16-7.02 (m, 3H), 6.89 (d, J=8.3 Hz, 2H), 6.60 (m, 2H), 5.61 (s, 1H), 5.05 (dd, J=12.7, 5.4 Hz, 1H), 3.80-3.75 (m, 2H), 3.65 (s, 2H), 3.61-3.52 (m, 5H), 3.48-3.45 (m, 10H), 3.18-3.17 8 m. 2H9, 3.08 (q, J=6.6 Hz, 2H), 2.87 (ddd, J=16.9, 13.7, 5.4 Hz, 1H), 2.63-2.53 (m, 2H), 2.30-2.26 (m, 4H), 2.04-1.99 (m, 1H), 1.90 (s, 1H), 1.75-1.57 (m, 5H), 1.28-1.23 (m, 4H), 1.23 (s, 1H), 0.87 (s, 4H).

¹³C NMR (126 MHz, DMSO) δ 173.3, 172.6, 170.6, 170.5, 169.4, 167.8, 167.6, 163.7, 162.1, 157.8, 148.6, 146.9, 136.7, 134.9, 132.6, 132.0, 130.2, 129.2, 123.5, 117.9, 114.7, 111.2, 109.7, 70.3, 70.2, 70.2, 70.1, 70.00, 69.4, 67.3, 62.4, 53.3, 52.9, 51.9, 49.7, 49.1, 49.0, 42.2, 38.8, 38.4, 36.8, 36.7, 34.8, 31.5, 29.5, 28.7, 22.6, 21.6.

LRMS (ESI) m/z: [M+H]+ calcd for C55H77N11O11; 1067.58 found, 1068.59.

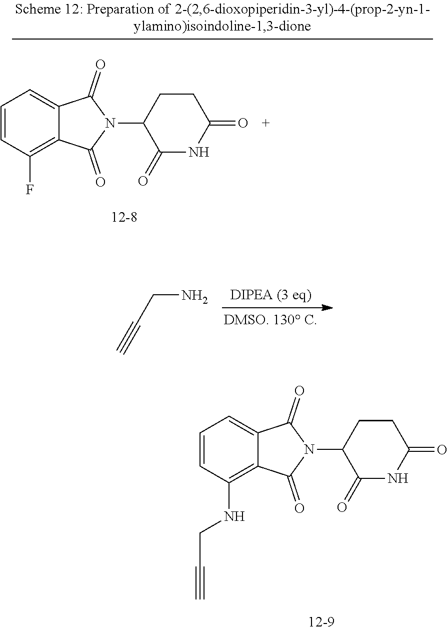

4-Fluoro-pomalidomide derivative 12-8 (320 mg, 1.16 mmol) was dissolved in DMSO (0.5 M), to the solution were subsequently added propargyl amine (64 mg, 1.16 mmol) and DIPEA (605 μl, 3.48 mmol). The resulting reaction mixture was stirred at 130° C. until completion (Monitored by TLC). The reaction mixture was quenched by addition of a saturated aqueous solution of NaHCO₃ and extracted into EtOAc (3×15 ml). The combined organic layers were dried over MgSO₄, filtrated, and evaporated. Product 12-9 was obtained as a yellow solid (280 mg, 78%).

¹H NMR (400 MHz, CDCl3) δ 8.00 (s, 1H), 7.57 (dd, J=8.5, 7.1 Hz, 1H), 7.20 (d, J=7.1 Hz, 1H), 7.03 (d, J=8.6 Hz, 1H), 6.44 (t, J=6.5 Hz, 1H), 4.92 (dd, J=12.2, 5.4 Hz, 1H), 4.09 (dd, J=6.1, 2.5 Hz, 2H), 2.95-2.67 (m, 3H), 2.27 (t, J=2.4 Hz, 1H), 2.20-2.07 (m, 1H).

¹³C NMR (101 MHz, CDCl3) δ 170.81, 169.24, 168.10, 167.44, 145.56, 136.16, 132.43, 117.17, 112.77, 111.42, 79.11, 72.20, 48.96, 32.33, 31.41, 22.76.

LRMS (ESI) m/z: [M+H]+ calcd for C16H13N3O4; 311.09 found, 312.09.

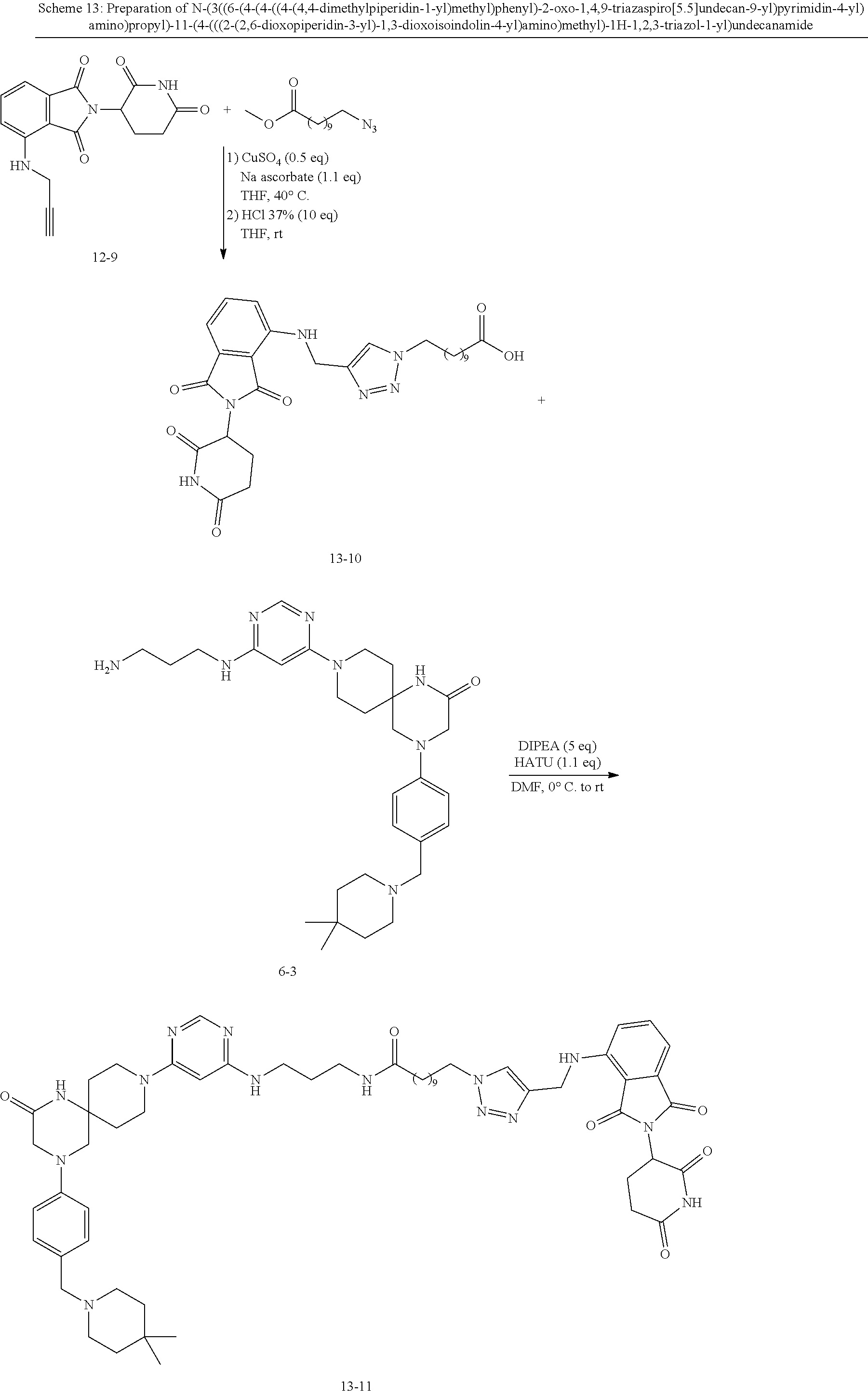

Compound 12-9 (100 mg, 0.32 mmol) was dissolved in THF (0.5 M), subsequently azido methyl ester (77 mg, 0.32 mmol), copper(II) sulfate (26 mg, 0.16 mmol) and sodium ascorbate (70 mg, 0.35 mmol) were added to the solution. The resulting reaction mixture was stirred at 40° C. until completion (Monitored by TLC), was then evaporated and the crude product was purified using flash column chromatography (SiO2; EtOAc/Hept=1:1) and obtained as a bright yellow solid (90 mg, 50%). The resulting methyl ester (90 mg, 0.16 mmol) was dissolved in THF (0.5 M) and HCl 37% (49 μL, 1.6 mmol) was added. The resulting reaction mixture was stirred at rt until completion (Monitored by TLC), was then evaporated and product 13-10 was involved in the next reaction without any further purification. Carboxylic acid 13-10 (83 mg, 0.15 mmol) was dissolved in DMF (0.5 M), the solution was cooled to 0° C. and DIPEA (134 μL, 0.768 mmol) was added. After 10 minutes HATU (65 mg, 0.169 mmol) was added and the solution was let stirring for 30 minutes after which amine 6-3 (80 mg, 0.15 mmol) was added. The resulting reaction mixture was stirred at rt until completion (Monitored by TLC). The reaction mixture was evaporated and the crude product was purified using flash column chromatography (SiO₂; DCM/MeOH=from 90:10 to 80:20), and 13-11 was obtained (7 mg, 5%).

¹H NMR (600 MHz, MeOD) δ 8.06 (t, J=5.6 Hz, 1H), 8.00 (s, 1H), 7.92 (s, 1H), 7.52 (t, J=8.5, 7.1, 4.4 Hz, 1H), 7.29 (d, 2H), 7.10-7.06 (m, 2H), 7.00 (t, J=6.1 Hz, 1H), 6.97 (d, 2H), 5.68 (s, 1H), 5.11-5.02 (dd, 1H), 4.65 (d, J=6.1 Hz, 2H), 4.36 (t, J=7.0 Hz, 2H), 3.83 (s, 2H), 3.72 (m, 2H), 3.55 (m, J=14.3, 4.0 Hz, 2H), 3.50 (m, J=4.3 Hz, 2H), 3.26 (q, J=6.6 Hz, 4H), 2.87-2.81 (m, 2H), 2.75 (dt, J=5.1, 2.6 Hz, 1H), 2.72 (t, J=1.1 Hz, 1H), 2.71-2.66 (m, 2H), 2.18 (t, J=8.7, 6.0 Hz, 3H), 2.13-2.07 (m, 1H), 2.07-2.02 (m, 1H), 1.94-1.88 (m, 2H), 1.88-1.80 (m, 2H), 1.78 (m, J=6.7 Hz, 2H), 1.60 (q, J=7.1, 6.3 Hz, 4H), 1.48 (, J=5.7 Hz, 4H), 1.32-1.29 (m, 16H), 0.97 (d, J=1.6 Hz, 6H).

¹³C NMR (151 MHz, MeOD) δ 176.55, 174.75, 171.72, 170.93, 170.55, 169.20, 164.62, 163.44, 163.31, 163.08, 162.85, 158.30, 150.59, 147.53, 147.48, 146.62, 137.15, 137.00, 133.96, 132.70, 128.76, 124.10, 118.38, 118.20, 115.82, 112.52, 112.43, 112.28, 112.05, 57.91, 57.76, 57.62, 57.48, 57.33, 54.42, 53.33, 52.40, 51.39, 50.65, 50.24, 41.63, 39.71, 39.02, 38.32, 38.00, 37.20, 33.11, 32.25, 31.75, 31.71, 31.18, 30.80, 30.77, 30.51, 30.34, 30.32, 30.30, 30.27, 30.23, 30.18, 30.15, 29.95, 29.91, 29.14, 27.33, 27.02, 23.81, 18.58, 17.62, 17.50, 17.37, 17.24, 17.12, 14.71, 14.58, 14.33, 14.20.

LRMS (ESI) m/z: [M+H]+ calcd for C56H77N14O6; 1040.61 found, 1040.61.

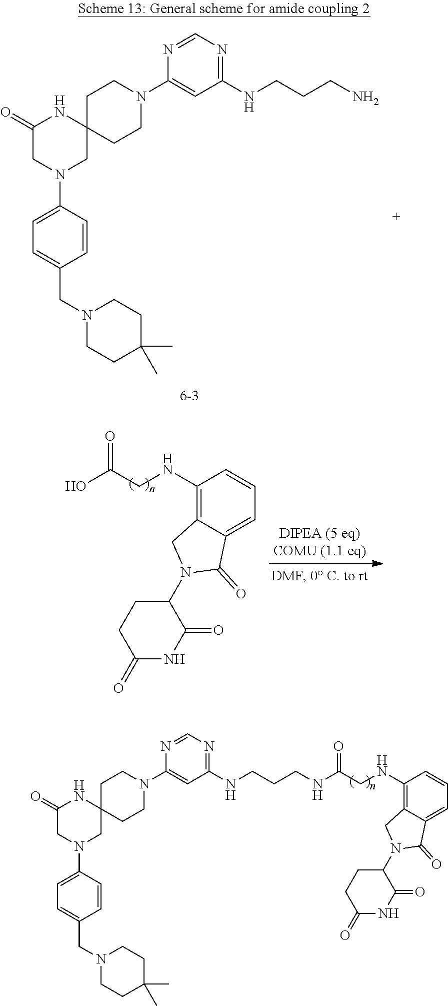

Carboxylic acid (1 equiv) was dissolved in DMF (0.5 M), the solution was cooled to 0° C. and DIPEA (5 equiv) was added. After 10 minutes COMU (1.1 equiv) was added and the solution was let stirred for 30 minutes after which amine 6-3 (1 equiv) was added. The resulting reaction mixture was stirred at rt until completion (Monitored by TLC).

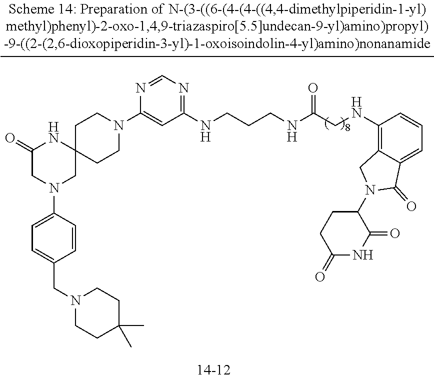

Compound 14-12 was prepared following the general amide coupling procedure 2 using amine 3 (63 mg, 0.12 mmol) and corresponding carboxylic acid (50 mg, 0.12 mmol). The reaction mixture was evaporated and the crude product was purified using two consecutive preparative thin layer chromatography (SiO₂; DCM/MeOH=84:16), and 14-12 was obtained (6 mg, 5%).

¹H NMR (400 MHz, DMSO) δ 11.00 (s, 1H), 8.27 (s, 1H), 7.98 (s, 1H), 7.78 (t, J=5.6 Hz, 1H), 7.27 (t, J=7.7 Hz, 1H), 7.14 (d, J=8.2 Hz, 2H), 6.91 (dd, J=7.9, 3.9 Hz, 3H), 6.72 (d, J=8.0 Hz, 1H), 6.64 (t, J=5.8 Hz, 1H), 5.61 (s, 1H), 5.54 (t, J=5.5 Hz, 1H), 5.10 (dd, J=13.2, 5.1 Hz, 1H), 4.22 (d, J=17.3 Hz, 1H), 4.12 (d, J=17.2 Hz, 1H), 3.77 (m, 2H), 3.66 (s, 2H), 3.17 (m, J=5.3 Hz, 2H), 3.08 (p, J=6.6 Hz, 4H), 2.92 (ddd, J=18.0, 13.4, 5.6 Hz, 2H), 2.69-2.57 (m, 2H), 2.36-2.23 (m, 2H), 2.04 (t, J=7.4 Hz, 2H), 1.72 (m, J=13.4 Hz, 2H), 1.58 (td, J=13.2, 12.8, 6.2 Hz, 6H), 1.47 (t, J=7.1 Hz, 2H), 1.31 (m, 2H), 1.25 (d, J=11.0 Hz, 12H), 0.88 (s, 6H).

¹³C NMR (151 MHz, MeOD) δ 176.53, 174.82, 172.54, 172.46, 170.82, 164.62, 163.41, 163.31, 163.08, 158.30, 150.81, 145.26, 132.93, 132.90, 130.63, 128.03, 115.77, 113.78, 111.81, 62.43, 57.76, 57.62, 57.48, 57.33, 54.40, 54.28, 53.58, 52.27, 50.27, 47.38, 44.52, 41.61, 39.83, 39.69, 39.55, 39.41, 39.27, 37.98, 37.94, 37.21, 35.59, 33.10, 32.45, 31.45, 30.82, 30.79, 30.76, 30.66, 30.51, 30.38, 30.33, 30.20, 30.13, 29.07, 28.12, 27.04, 27.01, 24.29, 23.77, 17.62, 17.37, 14.58, 14.47.

LRMS (ESI) m/z: [M+H]+ calcd for C51H72N11O5; 917.56 found, 918.57.

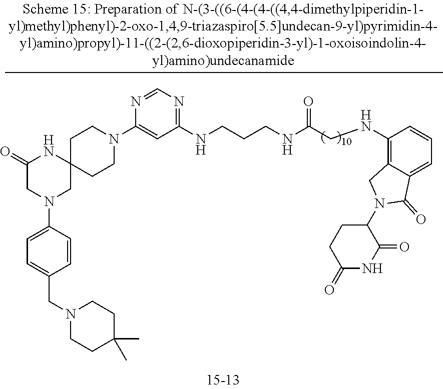

Compound 15-13 was prepared following the general amide coupling procedure 2 using amine 3 (47 mg, 0.090 mmol) and corresponding lenalidomide carboxylic acid (40 mg, 0.090 mmol). The reaction mixture was evaporated and the crude product was purified using flash column chromatography (SiO₂; DCM/MeOH=from 90:10 to 80:20), and 15-13 was obtained (36 mg, 42%).

¹H NMR (500 MHz, DMSO) δ 11.00 (s, 1H), 8.32 (s, 1H), 7.98 (s, 1H), 7.84 (t, J=5.7 Hz, 1H), 7.43-7.34 (m, 2H), 7.27 (t, J=7.7 Hz, 1H), 6.97 (d, J=8.3 Hz, 2H), 6.91 (d, J=7.4 Hz, 1H), 6.72 (d, J=8.1 Hz, 1H), 6.67 (m, J=6.1 Hz, 1H), 5.62 (s, 1H), 5.57 (t, J=5.5 Hz, 1H), 5.10 (dd, J=13.3, 5.1 Hz, 1H), 4.23 (d, J=17.2 Hz, 1H), 4.12 (d, J=17.2 Hz, 1H), 3.81-3.74 (m, 2H), 3.72 (s, 2H), 3.59 (m, J=11.8, 5.1 Hz, 2H), 3.51 (m, J=3.0 Hz, 2H), 3.45 (s, 2H), 3.17 (m, J=8.8, 5.4 Hz, 2H), 3.08 (dq, J=13.0, 6.5 Hz, 4H), 2.91 (ddp, J=15.1, 9.8, 5.2, 4.7 Hz, 2H), 2.61 (dt, J=17.5, 3.3 Hz, 2H), 2.29 (qd, J=13.2, 4.7 Hz, 2H), 2.03 (t, J=7.0 Hz, 2H), 1.70 (m, J=10.5, 4.2 Hz, 2H), 1.59 (tt, J=15.4, 8.3 Hz, 6H), 1.46 (m, J=12.8, 11.0, 5.5 Hz, 4H), 1.34 (m, J=7.0 Hz, 2H), 1.25 (m, J=21.7, 6.4 Hz, 14H), 0.94 (s, 5H).

¹³C NMR (126 MHz, DMSO) δ 172.95, 172.16, 171.27, 168.94, 166.91, 163.23, 161.64, 158.00, 157.75, 157.32, 143.80, 132.04, 129.23, 126.46, 118.54, 116.15, 113.76, 111.71, 109.88, 53.29, 52.42, 51.88, 50.73, 48.60, 47.66, 45.80, 42.75, 41.55, 38.01, 36.23, 35.47, 34.18, 31.25, 29.04, 28.94, 28.93, 28.82, 28.69, 28.55, 27.77, 26.67, 25.34, 22.83, 22.10, 18.61, 18.01, 16.74.

LRMS (ESI) m/z: [M+H]+ calcd for C53H76N11O5; 945.60 found, 946.60.

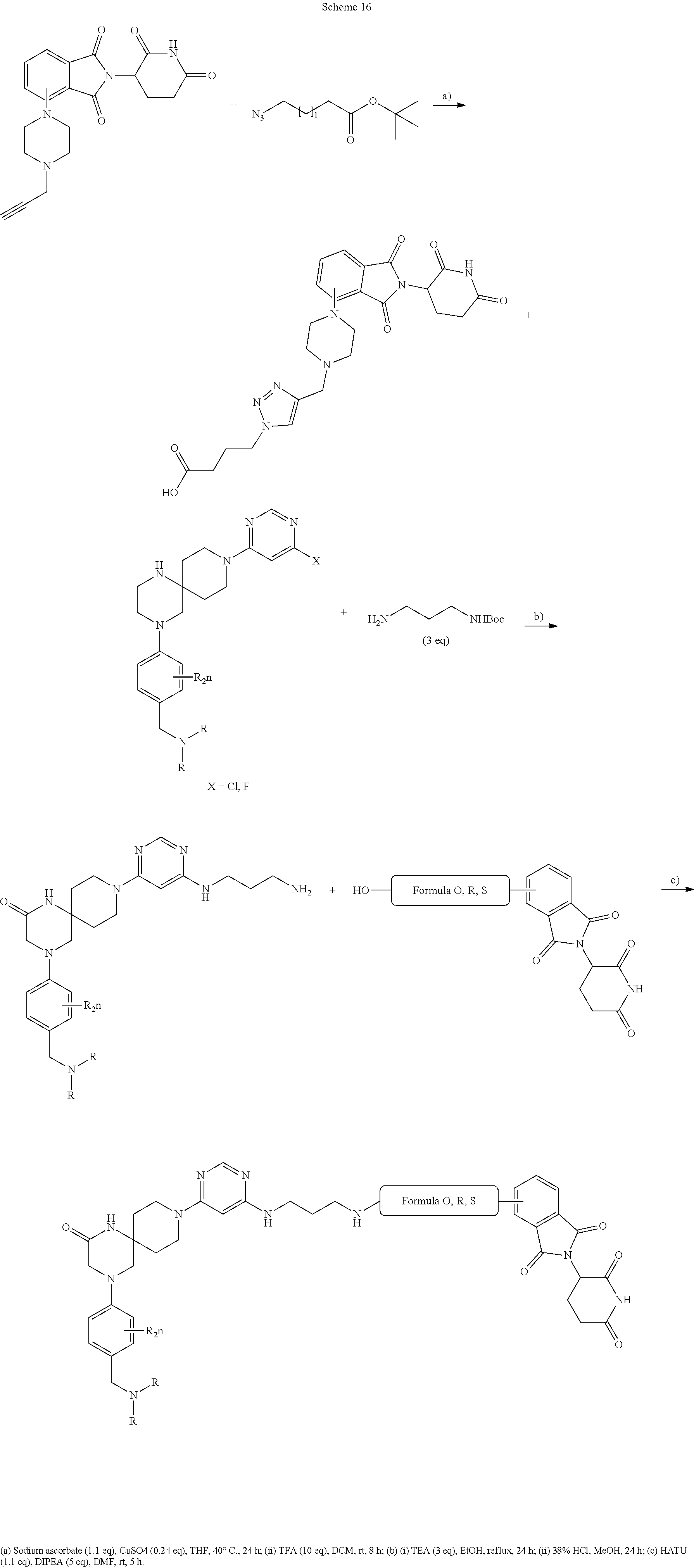

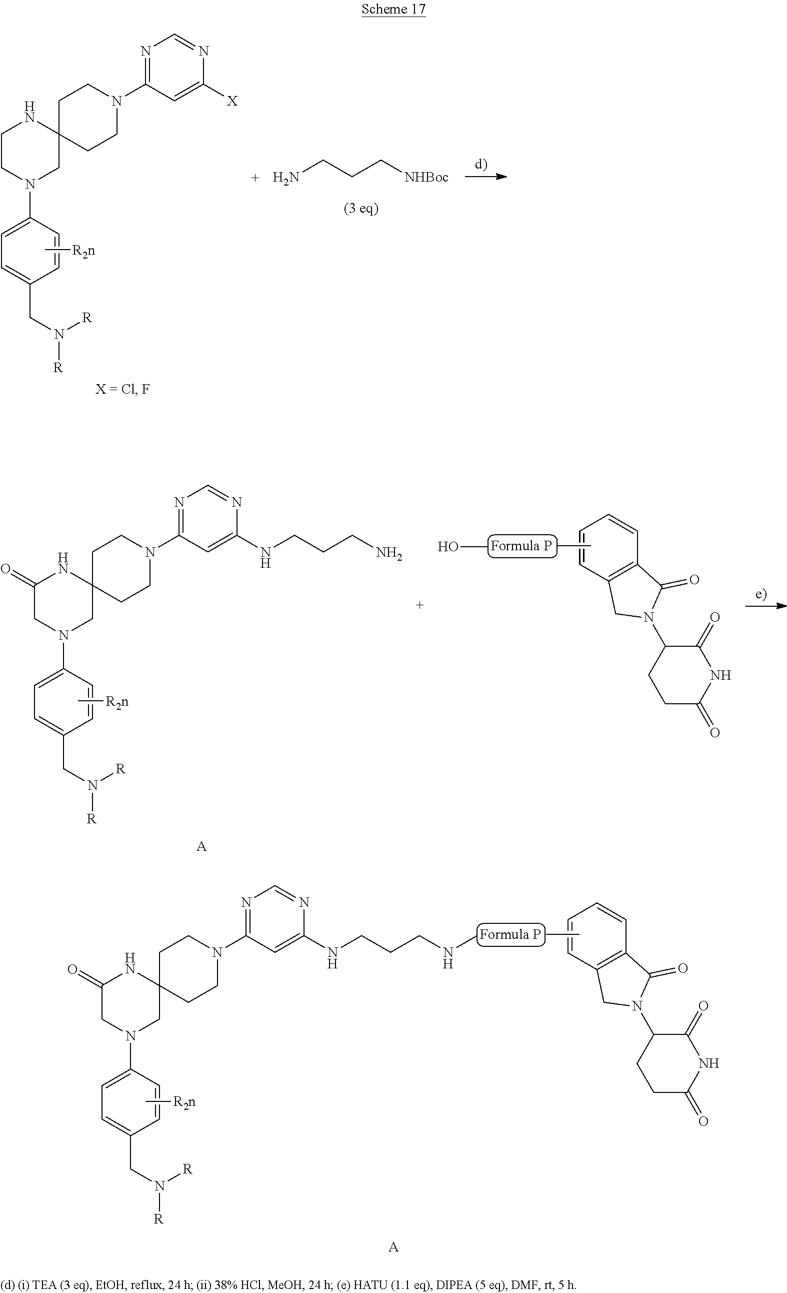

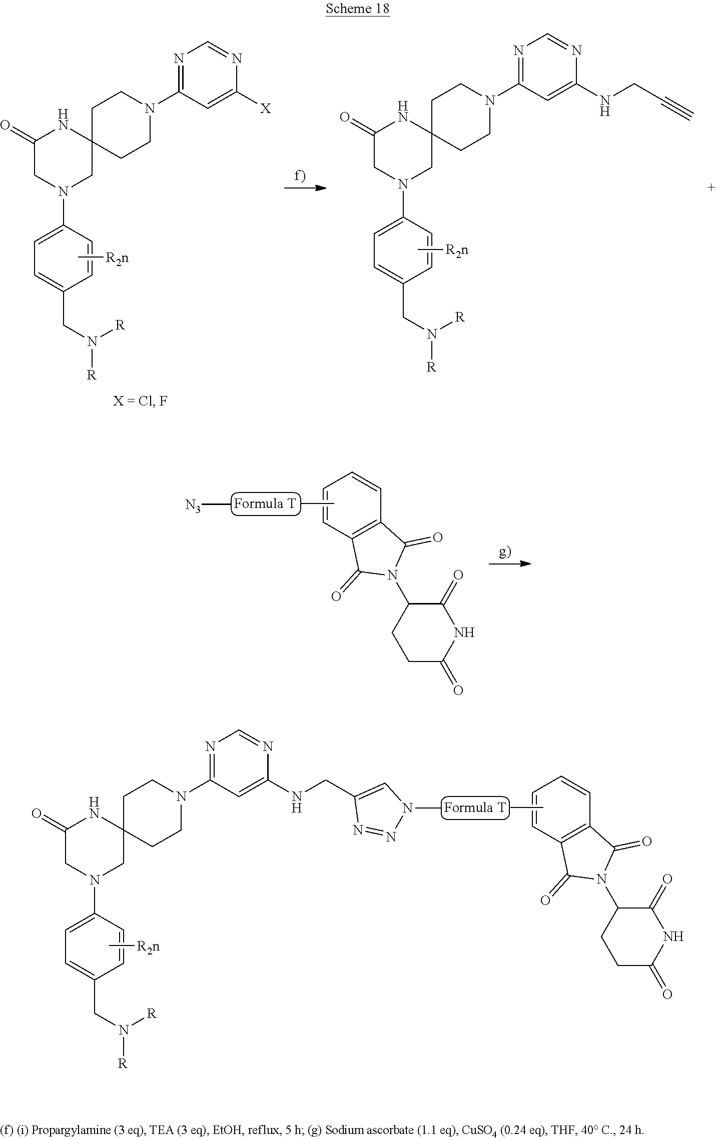

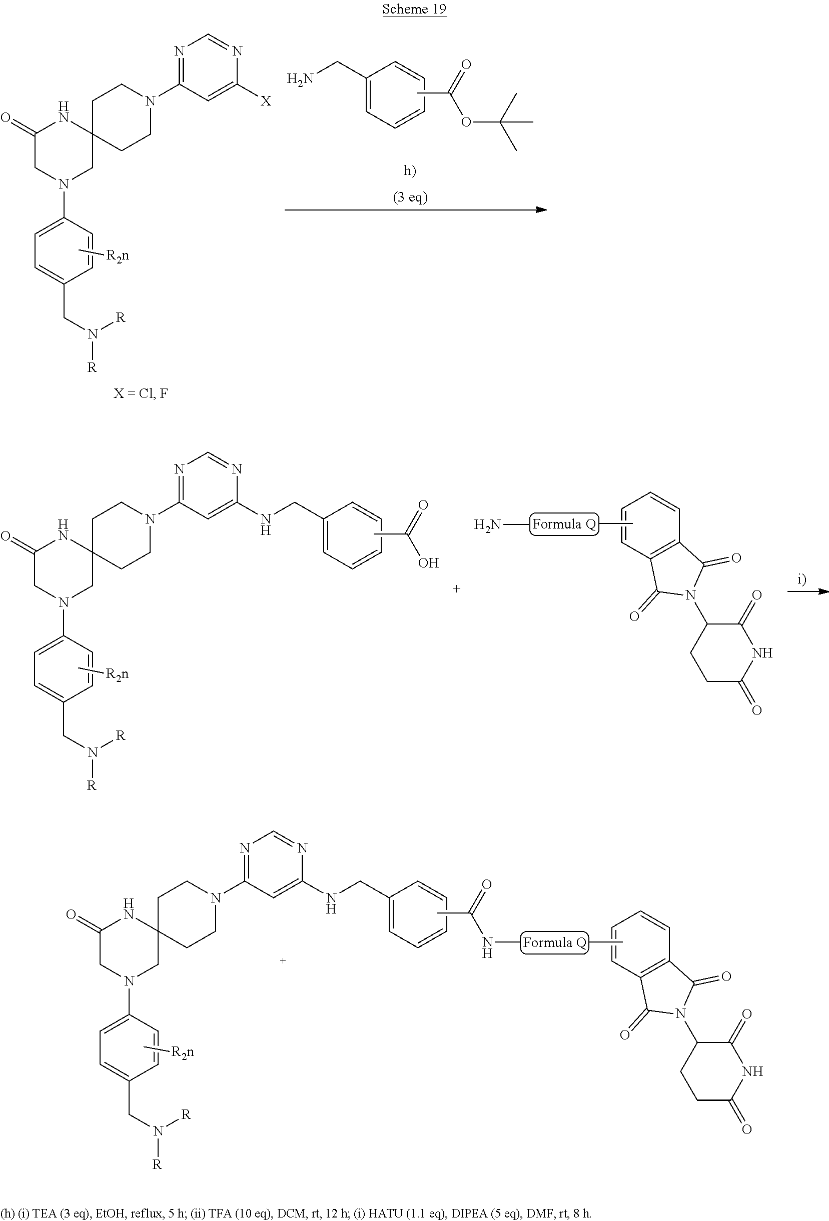

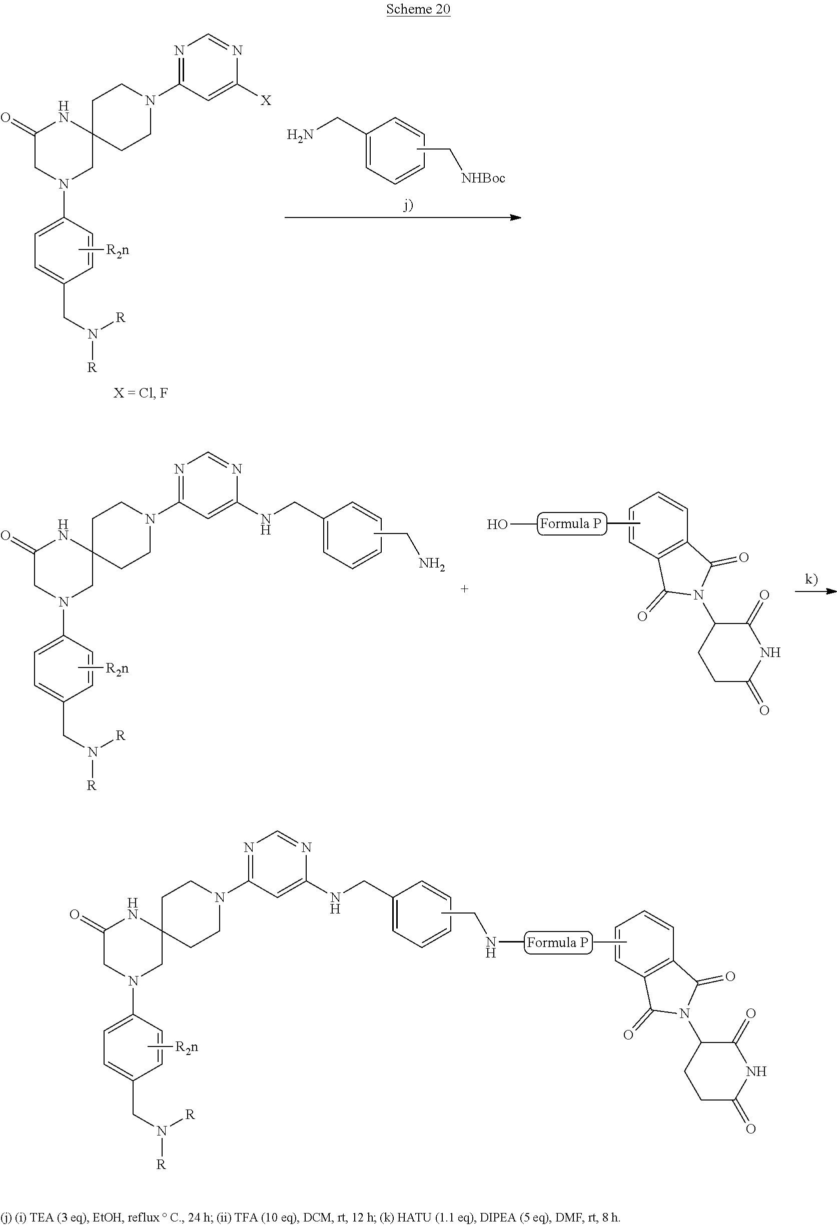

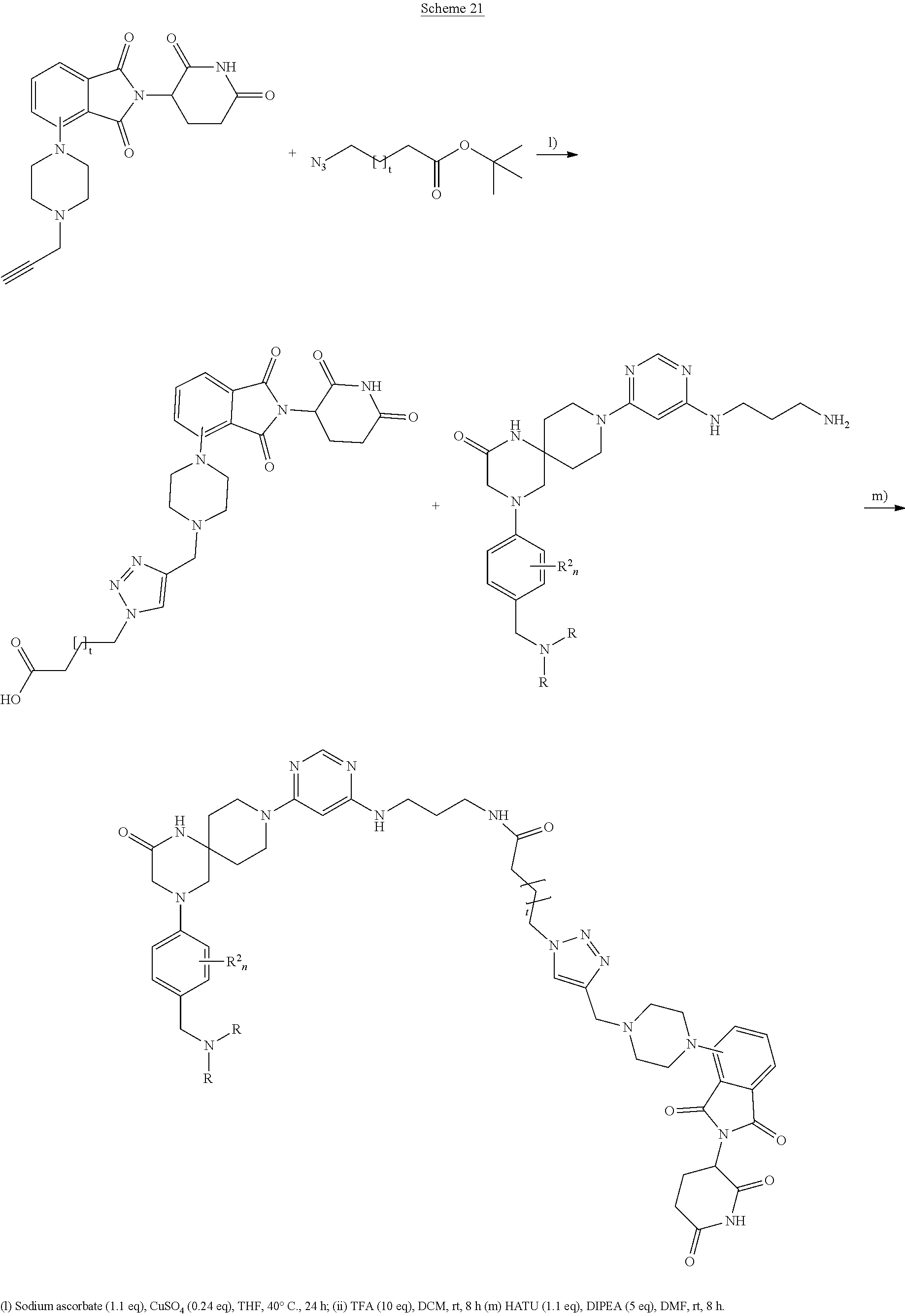

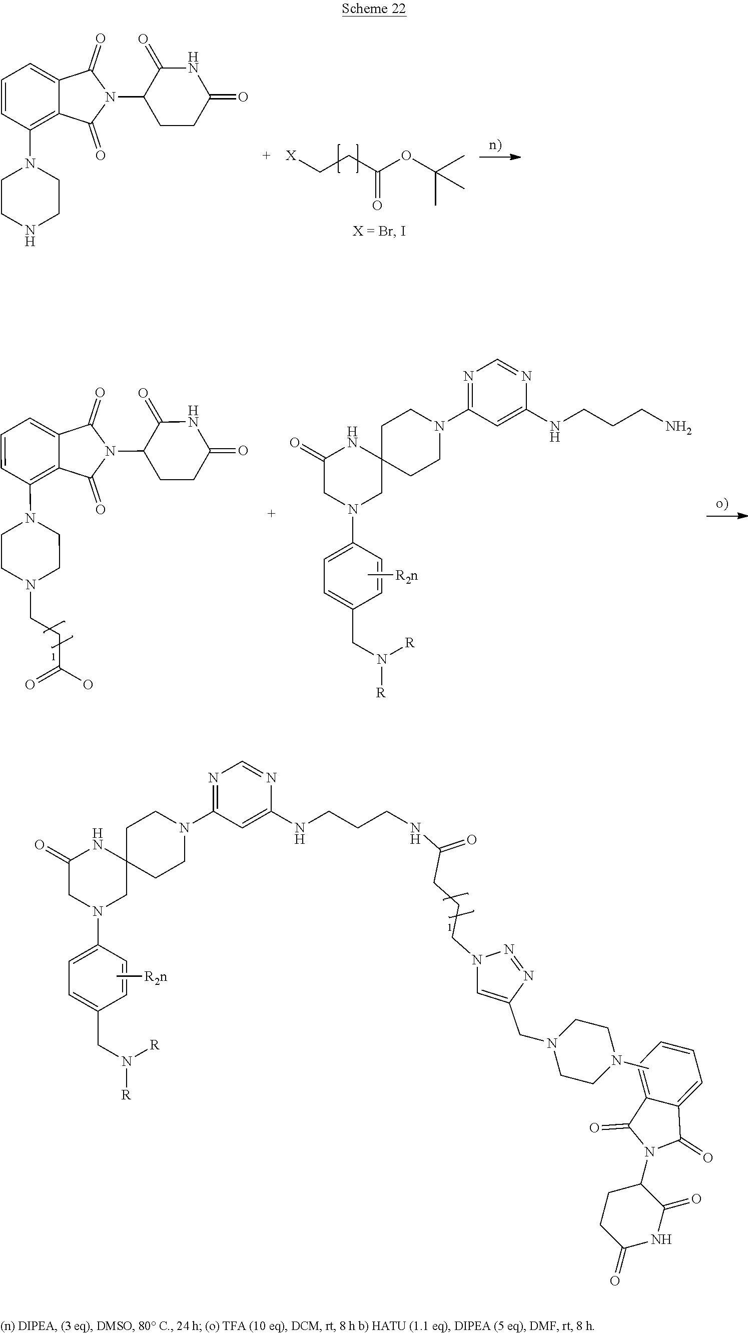

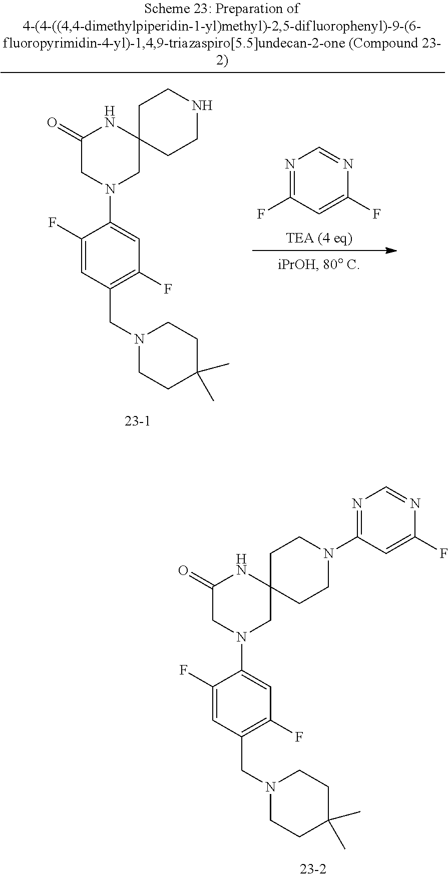 [SMILES: CC1(C)CCN(Cc2cc(F)c(N3CC(=O)NC4(CCNCC4)C3)cc2F)CC1 ; Fc1cc(F)ncn1 ; CC1(C)CCN(Cc2cc(F)c(N3CC(=O)NC4(CCN(c5cc(F)ncn5)CC4)C3)cc2F)CC1]

4-(4-((4,4-dimethylpiperidin-1-yl)methyl)-2,5-difluorophenyl)-1,4,9-triazaspiro[5.5]undecan-2-one 23-1 (100 mg, 0.27 mmol) was dissolved in iPrOH (0.5 M). To a stirred solution was subsequently added 4,6-difluoro pyrimidine (27 μL, 0.32 mmol) and TEA (150 μL, 1.01 mmol). The resulting reaction mixture was stirred at 80° C. (oil bath temperature) until full completion (Monitored by TLC). The volatiles were removed in vacuo and the crude product was purified using flash column chromatography (SiO₂; DCM/MeOH=9:1) providing 112 mg of the desired product (89%). ¹H NMR (400 MHz, DMSO) δ 8.30 (d, J=2.8 Hz, 1H), 8.22 (s, 1H), 7.15 (dd, J=13.5, 6.6 Hz, 1H), 6.93 (dd, J=11.5, 7.3 Hz, 1H), 6.58 (s, 1H), 4.00 (s, 2H), 3.60 (s, 2H), 3.58-3.47 (m, 2H), 3.42 (s, 2H), 3.28 (s, 2H), 2.33 (s, 3H), 1.83 (m, 2H), 1.68 (m, 2H), 1.30 (t, J=5.3 Hz, 4H), 0.87 (s, 6H). ¹³C NMR (101 MHz, DMSO) δ 172.5, 170.1, 167.1, 164.9, 164.8, 158.6, 158.4, 156.0, 152.0, 149.7, 107.1, 106.8, 85.9, 85.6, 55.4, 54.5, 53.3, 53.0, 49.5, 46.1, 38.7, 35.1, 28.6. LRMS (ESI) m/z: [M+H]+ calcd for C26H34F3N6O; 503.270 found, 503.276.

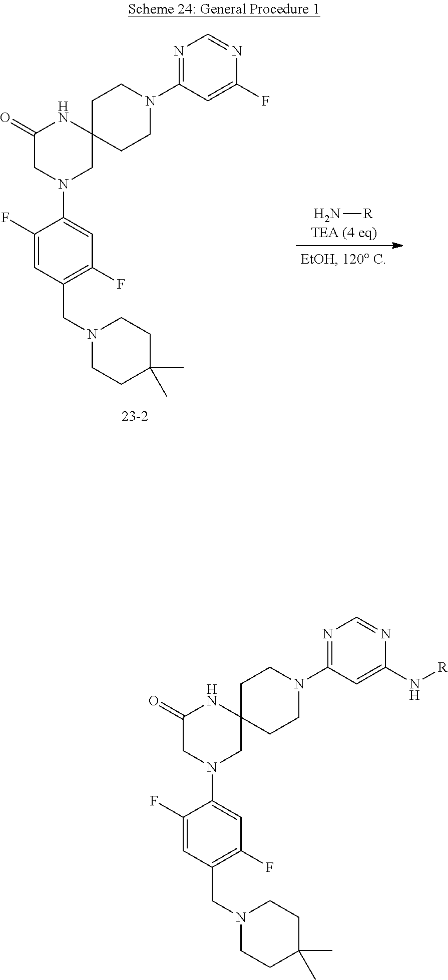

To a solution of compound 23-2 (1 eq) in EtOH (0.5 M) in a pressure vial was subsequently added amine (1.5 eq) and TEA (4 eq). The resulting reaction mixture was stirred at 120° C. (oil bath temperature) until completion (Monitored by TLC).

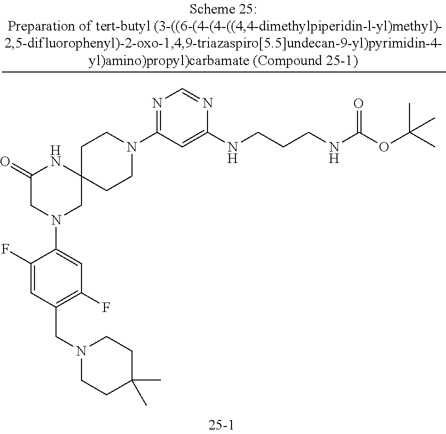 [SMILES: CC1(C)CCN(Cc2cc(F)c(N3CC(=O)NC4(CCN(c5cc(NCCCNC(=O)OC(C)(C)C)ncn5)CC4)C3)cc2F)CC1]

Compound 19 was prepared following General Procedure 1 using compound 23-2 (300 mg, 0.6 mmol) and tert-butyl (3-aminopropyl)carbamate (156 mg, 0.9 mmol). The volatiles were removed in vacuo and the crude product was purified using flash column chromatography (SiO₂; DCM/MeOH=90:10) providing 280 mg of desired product (71%). ¹H NMR (400 MHz, DMSO) δ 8.18 (s, 1H), 7.97 (s, 1H), 7.17 (s, 1H), 6.94 (dd, J=11.5, 7.3 Hz, 1H), 6.82 (t, J=5.7 Hz, 1H), 6.61 (t, J=5.7 Hz, 1H), 5.60 (s, 1H), 3.86 (d, J=13.5 Hz, 2H), 3.60 (s, 2H), 3.27 (s, 2H), 3.22-3.12 (m, 2H), 2.96 (q, J=6.6 Hz, 2H), 2.37 (s, 4H), 1.77 (dt, J=14.2, 4.2 Hz, 2H), 1.70-1.52 (m, 5H), 1.37 (s, 9H), 1.34-1.28 (m, 4H), 0.88 (s, 6H). ¹³C NMR (101 MHz, DMSO) δ 166.5, 163.2, 161.6, 157.3, 155.7, 117.9, 106.5, 106.3, 77.5, 54.7, 52.8, 52.7, 49.0, 38.0, 37.7, 34.6, 29.5, 28.3, 28.1. LRMS (ESI) m/z: [M+H]+ calcd for C34H51F2N8O3; 657.404 found, 657.405.

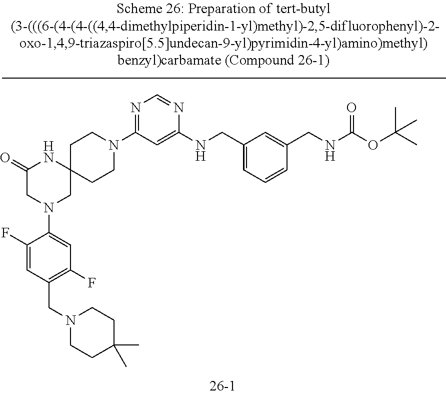 [SMILES: CC1(C)CCN(Cc2cc(F)c(N3CC(=O)NC4(CCN(c5cc(NCc6cccc(CNC(=O)OC(C)(C)C)c6)ncn5)CC4)C3)cc2F)CC1]

Compound 26-1 was prepared following General Procedure 1 using compound 23-2 (50 mg, 0.1 mmol) and 1-(N-Boc-aminomethyl)-3-(aminomethyl)benzene (35 mg, 0.15 mmol). The volatiles were removed in vacuo and the crude product was purified using flash column chromatography (SiO₂; DCM/MeOH=90:10) providing 51 mg of the desired product (71%). ¹H NMR (400 MHz, CDCl₃) δ 8.16 (s, 1H), 7.30 (t, J=7.5 Hz, 1H), 7.23-7.18 (m, 3H), 7.12 (dd, J=12.8, 6.6 Hz, 1H), 6.60-6.56 (m, 2H), 5.43 (s, 1H), 5.20 (s, 1H), 4.89 (s, 1H), 4.53 (d, J=5.8 Hz, 2H), 4.30 (d, J=6.0 Hz, 2H), 3.73-3.66 (m, 4H), 3.54 (m, 2H), 3.50 (s, 2H), 3.27 (s, 2H), 2.41 (s, 3H), 1.93 (m, 2H), 1.77 (m, 2H), 1.44 (s, 8H), 1.40 (t, J=7.4, 6.5 Hz, 4H), 0.90 (s, 6H). ¹³C NMR (101 MHz, CDCl₃) δ 167.7, 163.2, 162.5, 157.9, 155.9, 139.7, 138.6, 129.1, 126.5, 126.2, 126.1, 105.8, 105.5, 81.3, 56.4, 54.7, 53.5, 53.0, 49.7, 45.7, 44.5, 40.4, 38.5, 35.2, 29.7, 28.4, 28.4. LRMS (ESI) m/z: [M+H]+ calcd for C39H53F₂N803; 719.420 found, 719.422.

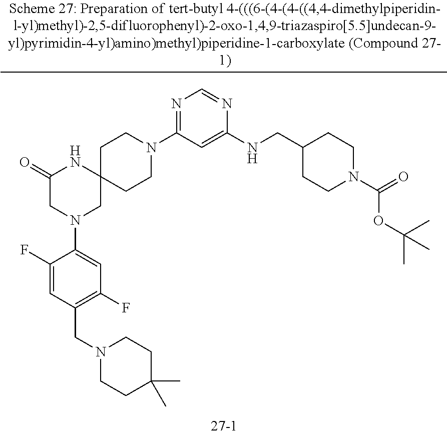 [SMILES: CC1(C)CCN(Cc2cc(F)c(N3CC(=O)NC4(CCN(c5cc(NCC6CCN(C(=O)OC(C)(C)C)CC6)ncn5)CC4)C3)cc2F)CC1]

Compound 21 was prepared following the General procedure 1 using compound 18 (50 mg, 0.1 mmol) and tert-butyl 4-(aminomethyl)piperidine-1-carboxylate (42 mg, 0.15 mmol). he volatiles were removed in vacuo and the crude product was purified using flash column chromatography (SiO₂; DCM/MeOH=90:10) providing 55 mg of the desired product (79%). ¹H NMR (400 MHz, CDCl₃) δ 8.14 (s, 1H), 7.11 (dd, J=12.9, 6.6 Hz, 1H), 6.67 (m, 1H), 6.58 (dd, J=10.9, 7.1 Hz, 1H), 5.43 (s, 1H), 4.87 (s, 1H), 4.13 (s, 2H), 3.78-3.68 (m, 4H), 3.60 (m, 2H), 3.48 (s, 2H), 3.28 (s, 2H), 3.14 (t, J=6.1 Hz, 2H), 2.70 (t, J=12.7 Hz, 2H), 2.44-2.37 (m, 4H), 1.97 (m, 2H), 1.82 (m, 2H), 1.77-1.69 (m, 3H), 1.45 (s, 9H), 1.39 (t, J=5.6 Hz, 4H), 1.28-1.11 (m, 2H), 0.91 (s, 6H). ¹³C NMR (101 MHz, CDCl₃) δ 167.8, 163.4, 162.4, 157.8, 156.0, 154.8, 152.2, 137.3, 118.4, 118.2, 105.9, 105.6, 80.7, 79.5, 56.5, 54.7, 53.4, 53.0, 49.8, 47.2, 40.2, 38.6, 36.4, 35.4, 30.0, 28.5, 28.4. LRMS (ESI) m/z: [M+H]+ calcd for C37H55F2N8O3; 697.440 found, 697.438.

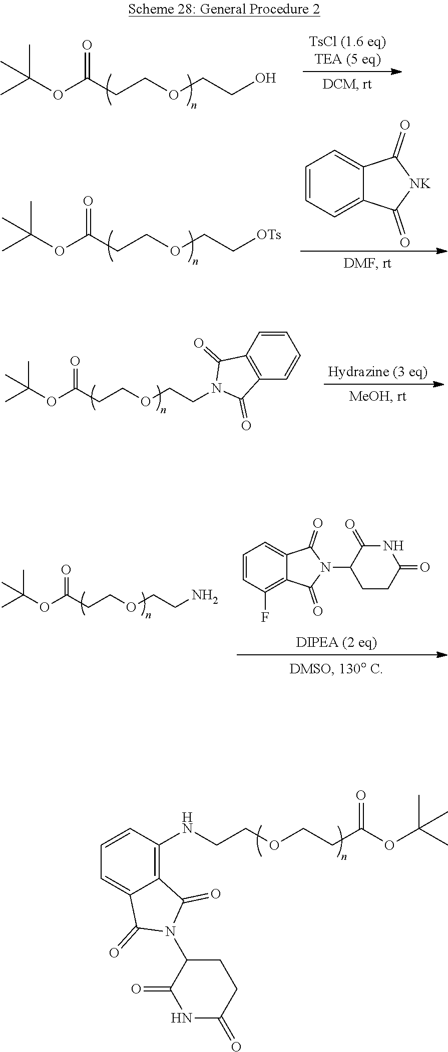

To a stirred solution of corresponding tert-butylester (1 eq) in DCM (0.5 M) was subsequently added TsCl (1.6 eq) and TEA (5 eq). The resulting reaction mixture was stirred at rt until full completion (Monitored by TLC). The volatiles were removed in vacuo and the product was involved in the next step without further purification.

To a stirred solution of corresponding tosylated tert-butylester in DMF (0.5 M), was added potassium phthalimide (2 eq). The resulting reaction mixture was stirred at rt until full completion (Monitored by TLC). The volatiles were then removed in vacuo and the product was used in the next step without further purification.

The crude was dissolved in methanol (0.5 M), and hydrazine hydrate 50-60% (3 eq). The resulting reaction mixture was stirred at rt until full completion (Monitored by TLC). To a resulting mixture was added DCM and the formed precipitate was removed by filtration. The filtrate was concentrated in vacuo and the resulting product was involved in the next step without any further purification.

To a stirred solution of 4-fluoro-thalidomide (1 eq) in DMSO (0.5 M) was subsequently added corresponding tert-butyl ester (1 eq) and DIPEA (2 eq). The resulting reaction mixture was stirred at 130° C. (oil bath temperature) until completion (Monitored by TLC).

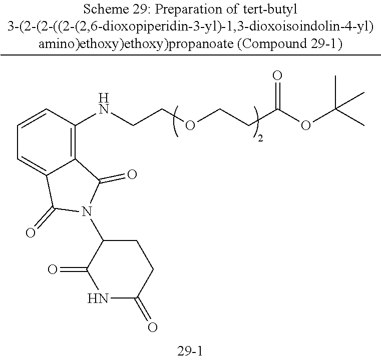

Compound 29-1 was prepared following General Procedure 2 using 4-fluoro-thalidomide (82 mg, 0.3 mmol) and the corresponding tert-butylester (70 mg, 0.3 mmol). The volatiles were then removed in vacuo and the crude product was purified using flash column chromatography (SiO₂; EtOAc/Hept=from 80:20 to 90:10) providing 64 mg of the desired product (57%). ¹H NMR (400 MHz, CDCl₃) δ 8.1 (s, 1H), 7.52 (m, J=8.4, 7.1 Hz, 1H), 7.12 (d, J=7.1 Hz, 1H), 6.97 (d, J=8.5 Hz, 1H), 6.47 (t, J 5.6 Hz, 1H), 4.85 (dd, J 12.4, 5.3 Hz, 1H), 3.74 (m, 4H), 3.65 (m, 4H), 3.46 (m, 2H), 2.89 (m, 1H), 2.86 (m, 2H), 2.53 (t, J=6.6 Hz, 2H), 2.18-2.10 (m, 1H), 1.40 (s, 9H).

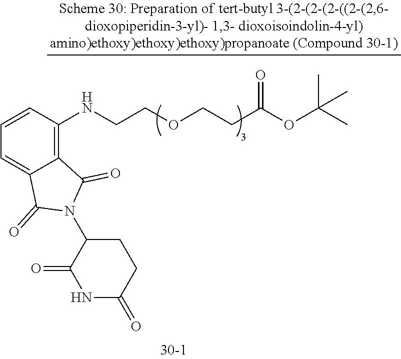

Compound 30-1 was prepared following General Procedure 2 using 4-fluoro-thalidomide (68 mg, 0.25 mmol) and the corresponding tert-butyl ester (69 mg, 0.25 mmol). The volatiles were then removed in vacuo and the crude product was purified using flash column chromatography (SiO₂; EtOAc/Hept=from 80:20 to 90:10) providing 52 mg of the desired product (39%). ¹H NMR (400 MHz, CDCl₃) δ 8.12 (s, 1H), 7.55 (t, J=7.6 Hz, 1H), 7.13 (d, J=6.8 Hz, 1H), 6.94 (d, J=8.3 Hz, 1H), 6.51 (s, 1H), 4.94 (m, 1H), 3.73 (m, 13H), 3.50 (m, 2H), 2.92-2.70 (m, 3H), 2.50 (t, J=6.2 Hz, 2H), 2.15 (m, 1H), 1.44 (s, 9H)

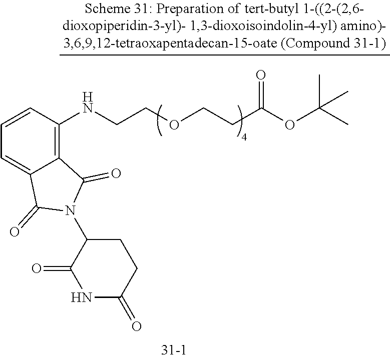

Compound 31-1 was prepared following General Procedure 2 using 4-fluoro-thalidomide (53 mg, 0.2 mmol) and the corresponding tert-butyl ester (62 mg, 0.2 mmol). The volatiles were then removed in vacuo and the crude product was purified using flash column chromatography (SiO₂; EtOAc/Hept=from 80:20 to 90:10) affording 64 mg of the desired product (57%). ¹H NMR (400 MHz, CDCl₃) δ 8.11 (s, 1H), 7.44 (dd, J=7.32, 8.34 Hz, 1H), 7.04 (d, J=7.11 Hz, 1H), 6.83 (d, J=8.54 Hz, 1H), 6.42 (m, 1H), 4.8 (s, 1H), 3.58 (m, 18H), 3.42-3.35 (m, 2H), 2.72 (s, 3H), 2.45-2.39 (m, 2H), 1.98 (m, 1H), 1.43 (m, 9H).

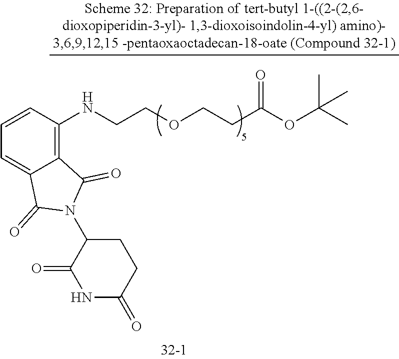

Compound 32-1 was prepared following General Procedure 2 using 4-fluoro-thalidomide (56 mg, 0.2 mmol) and the corresponding tert-butyl ester (74 mg, 0.2 mmol). The volatiles were then removed in vacuo and the crude product was purified using flash column chromatography (SiO₂; EtOAc/Hept=from 80:20 to 90:10) affording 61 mg of the desired product (49%). ¹H NMR (400 MHz, CDCl₃) δ 8.19 (s, 1H), 7.46 (t, J=7.7 Hz, 1H), 7.13 (d, J=7.0 Hz, 1H), 6.90 (d, J=8.4 Hz, 1H), 6.50 (m, 1H), 4.92 (m, 1H), 3.76-3.58 (m, 18H), 3.46 (m, 2H), 2.94-2.73 (m, 3H), 2.52 (t, J=6.4 Hz, 2H), 2.14 (m, 1H), 1.43 (m, 9H).

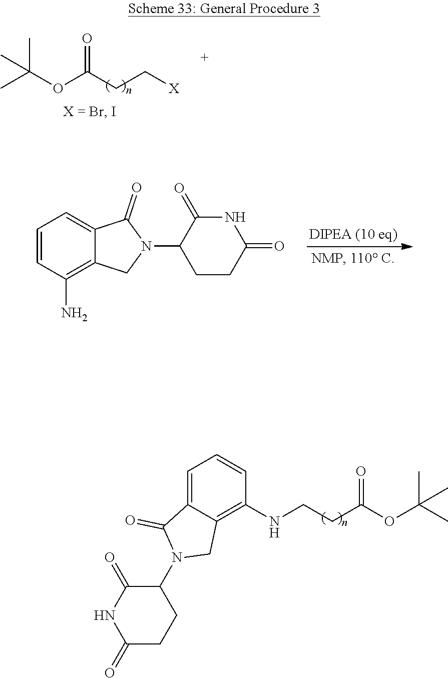

To a stirred solution of lenalidomide (1 eq) in NMP (0.5 M) was subsequently added corresponding tert-butyl ester (1 eq) and DIPEA (10 eq). The resulting reaction mixture was stirred at 110° C. (oil bath temperature) until full completion (Monitored by TLC).

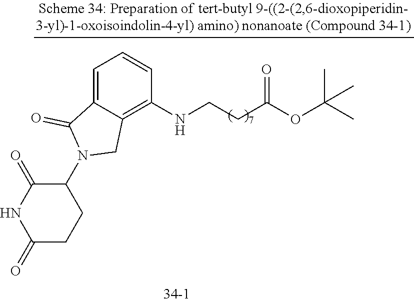

Compound 34-1 was prepared following General Procedure 3 using lenalidomide (267 mg, 1.03 mmol) and the corresponding tert-butyl ester (303 mg, 1.03 mmol). The volatiles were then removed in vacuo and the crude product was purified using flash column chromatography (SiO₂; EtOAc/Hept=70:30) providing 200 mg of the desired product (41%). ¹H NMR (400 MHz, DMSO) δ 11.00 (s, 1H), 7.27 (t, J=7.7 Hz, 1H), 6.92 (d, J=7.3 Hz, 1H), 6.73 (d, J=8.0 Hz, 1H), 5.55 (t, J=5.5 Hz, 1H), 5.10 (dd, J=13.3, 5.1 Hz, 1H), 4.26-4.08 (m, 2H), 3.10 (q, J=6.6 Hz, 2H), 2.92 (ddd, J=18.3, 13.6, 5.4 Hz, 1H), 2.73-2.57 (m, 2H), 2.36-2.22 (m, 2H), 2.16 (td, J=7.3, 3.7 Hz, 2H), 2.08-1.97 (m, 1H), 1.56 (m, 2H), 1.47 (m, 2H), 1.38 (s, 9H), 1.25 (m, 8H). ¹³C NMR (101 MHz, CDCl₃) δ 173.9, 172.2, 170.2, 166.3, 147.5, 131.6, 129.8, 125.72, 116.7, 116.2, 80.3, 55.4, 48.2, 43.6, 35.2, 31.1, 29.3, 28.4, 27.7, 27.2, 26.4, 24.3. LRMS (ESI) m/z: [M+H]+ calcd for C22H30N3O5; 416.22 found, 416.26.

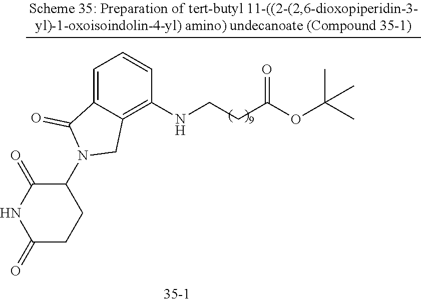

Compound 35-1 was prepared following General Procedure 3 using lenalidomide (245 mg, 0.95 mmol) and the corresponding tert-butyl ester (304 mg, 0.95 mmol). The volatiles were then removed in vacuo and the crude product was purified using flash column chromatography (SiO₂; EtOAc/Hept=70:30) providing 125 mg of the desired product (26%). ¹H NMR (400 MHz, DMSO) δ 11.00 (s, 1H), 7.27 (t, J=7.7 Hz, 1H), 6.95-6.88 (m, 1H), 6.73 (d, J=8.0 Hz, 1H), 5.55 (t, J=5.5 Hz, 1H), 5.10 (dd, J=13.2, 5.1 Hz, 1H), 4.28-4.07 (m, 2H), 3.10 (q, J=6.6 Hz, 2H), 2.92 (ddd, J=18.0, 13.5, 5.3 Hz, 1H), 2.72-2.58 (m, 2H), 2.36-2.23 (m, 2H), 2.16 (t, J=7.3 Hz, 2H), 2.07-1.98 (m, 1H), 1.57 (m, 2H), 1.46 (m, 2H), 1.38 (s, 9H), 1.24 (m, 14H). ¹³C NMR (101 MHz, CDCl3) δ 173.7, 172.5, 170.3, 165.9, 148.1, 131.7, 130.1, 125.6, 117.2, 116.4, 80.5, 56.3, 49.1, 43.6, 35.4, 31.3, 30.8, 29.7, 29.1, 28.2, 27.7, 27.3, 26.7, 23.3. LRMS (ESI) m/z: [M+H]+ calcd for C28H42N3O5; 500.31 found, 500.34.

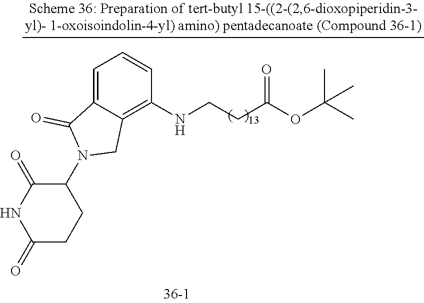

Compound 36-1 was prepared following General Procedure 3 using lenalidomide (264 mg, 1.02 mmol) and the corresponding tert-butyl ester (432 mg, 1.02 mmol). The volatiles were then removed in vacuo and the crude product was purified using flash column chromatography (SiO₂; EtOAc/Hept=70:30) affording 125 mg of the desired product (22%). ¹H NMR (400 MHz, CDCl₃) δ 7.98 (s, 1H), 7.36 (t, J=7.7 Hz, 1H), 6.80 (dd, J=8.0, 0.9 Hz, 1H), 5.25 (dd, J=13.2, 5.2 Hz, 1H), 4.36-4.09 (m, 2H), 4.05 (t, J=6.8 Hz, 1H), 3.20 (t, J=7.3 Hz, 2H), 2.92-2.78 (m, 2H), 2.36-2.26 (m, 2H), 2.20 (t, J=7.5 Hz, 2H), 1.69-1.53 (m, 6H), 1.44 (s, 9H), 1.31-1.22 (m, 18H). ¹³C NMR (101 MHz, DMSO) δ 174.4, 173.1, 170.5, 165.6, 149.7, 131.2, 129.5, 126.2, 117.1, 116.3, 79.8, 56.1, 48.4, 43.8, 36.7, 33.4, 30.7, 29.5, 29.5, 29.2, 28.4, 28.1, 27.7, 27.2, 26.9, 26.7, 25.5, 24.8. LRMS (ESI) m/z: [M+H]+ calcd for C32H50N3O5; 556.38 found, 556.41.

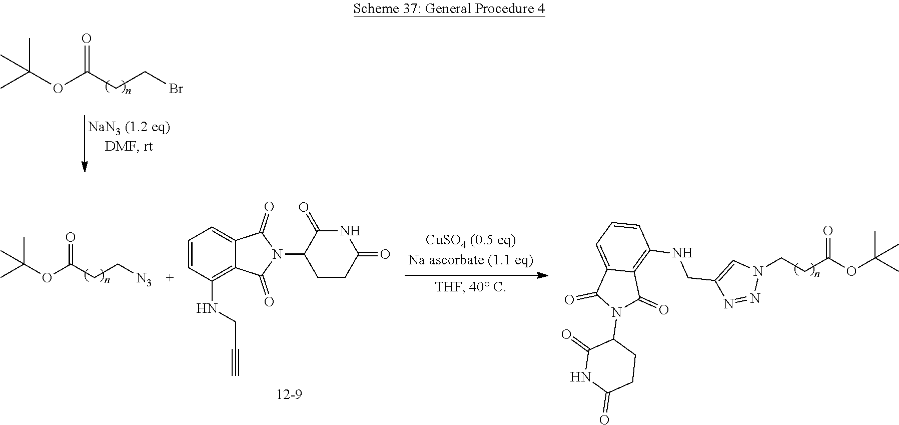

To a stirred solution of tert-butyl ester (1 eq) in DMF (0.5 M) was added sodium azide (1.2 eq). The resulting reaction mixture was stirred at rt until full completion (Monitored by ¹H NMR). The volatiles were then removed in vacuo, taken up with DCM and filtered through the filter paper. The product was involved in the next step without further purification.

Compound 12-9 (1 eq) was dissolved in THF (0.5 M) followed by addition of corresponding tert-butyl ester (1 eq), anhydrous CuSO₄ (0.5 eq) and sodium ascorbate (1.1 eq), respectively. The resulting reaction mixture was stirred at 40° C. (oil bath temperature) until full completion (Monitored by TLC).

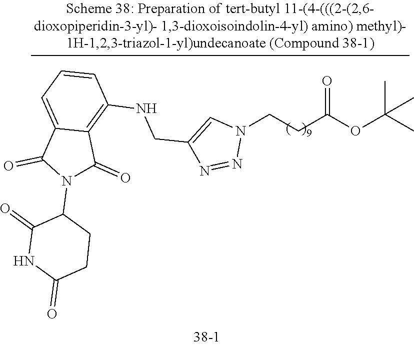

Compound 38-1 was prepared following General Procedure 4 using compound 12-9 (80 mg, 0.26 mmol) and the corresponding tert-butyl ester (73 mg, 0.26 mmol). The volatiles were then removed in vacuo and the crude product was purified using flash column chromatography (SiO₂; EtOAc/Hept=from 40:60 to 50:50) providing 85 mg of the desired product (55%). ¹H NMR (400 MHz, CDCl₃) δ 7.99 (s, 1H), 7.49 (dd, J=8.5, 7.1 Hz, 1H), 7.45 (s, 1H), 7.14 (d, J=7.1 Hz, 1H), 7.00 (d, J=8.5 Hz, 1H), 6.68 (t, J=6.0 Hz, 1H), 4.92 (dd, J=12.2, 5.4 Hz, 1H), 4.64 (d, J=6.0 Hz, 2H), 4.31 (t, J=7.3 Hz, 2H), 2.95-2.67 (m, 3H), 2.19 (t, J=7.5 Hz, 2H), 2.13 (m, 1H), 1.88 (t, J=7.2 Hz, 2H), 1.44 (s, 9H), 1.33-1.21 (m, 14H). ¹³C NMR (101 MHz, CDCl₃) δ 173.2, 172.5, 171.0, 164.9, 163.4, 142.7, 140.8, 132.5, 131.6, 122.7, 121.8, 119.8, 113.4, 82.3, 52.5, 49.8, 38.3, 35.1, 30.3, 29.7, 29.2, 28.6, 27.7, 26.8, 26.1, 23.2. LRMS (ESI) m/z: [M+H]+ calcd for C31H43N6O6; 595.32 found, 595.35.

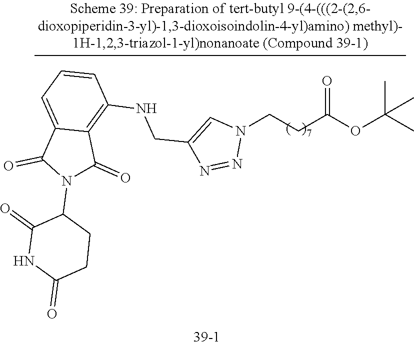

Compound 39-1 was prepared following General Procedure 4 using compound 12-9 (85 mg, 0.27 mmol) and the corresponding tert-butyl ester (70 mg, 0.27 mmol). The volatiles were then removed in vacuo and the crude product was purified using flash column chromatography (SiO₂; EtOAc/Hept=from 40:60 to 50:50) affording 73 mg of the desired product (47%). ¹H NMR (400 MHz, CDCl₃) δ 8.06 (s, 1H), 7.49 (dd, J=8.5, 7.2 Hz, 1H), 7.44 (s, 1H), 7.14 (d, J=7.2, 0.6 Hz, 1H), 7.00 (d, J=8.5 Hz, 1H), 6.67 (t, J=5.9 Hz, 1H), 4.92 (dd, J=12.1, 5.4 Hz, 1H), 4.64 (d, J=6.0 Hz, 2H), 4.31 (t, J=7.9, 6.5 Hz, 2H), 2.94-2.67 (m, 3H), 2.21-2.15 (m, 2H), 2.15-2.09 (m, 1H), 1.92-1.83 (m, 1H), 1.43 (s, 9H), 1.30-1.22 (m, 11H). ¹³C NMR (101 MHz, CDCl₃) δ 173.8, 172.3, 170.3, 165.5, 164.8, 143.4, 141.0, 131.4, 130.3, 123.1, 121.4, 120.6, 111.5, 80.25, 51.7, 50.2, 37.3, 35.1, 30.5, 29.2, 28.6, 27.7, 26.4, 24.3. LRMS (ESI) m/z: [M+H]+ calcd for C29H39N6O6; 567.29 found, 597.30.

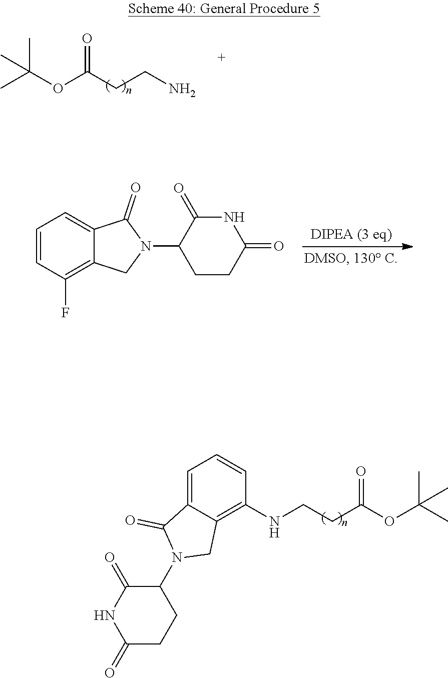

To a stirred solution of 4-fluoro-thalidomide (1 eq) in DMSO (0.5 M) was subsequently added corresponding tert-butyl ester (1 eq) and DIPEA (3 eq). The resulting reaction mixture was stirred at 130° C. (oil bath temperature) until full completion (Monitored by TLC).

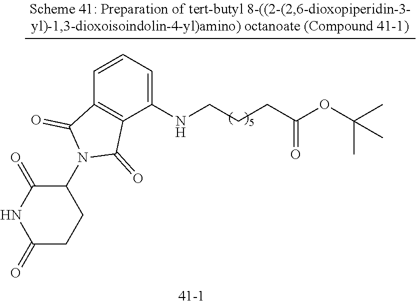

Compound 41-1 was prepared following General Procedure 5 using 4-fluoro-thalidomide (258 mg, 0.93 mmol) and the corresponding tert-butyl ester (201 mg, 0.93 mmol). The volatiles were then removed in vacuo and the crude product was purified using flash column chromatography (SiO₂; EtOAc/Hept=2:3) providing 251 mg of the desired product (57% yield). ¹H NMR (400 MHz, CDCl₃) δ 8.07 (s, 1H), 7.49 (dd, J=8.5, 7.1 Hz, 1H), 7.08 (d, J=7.0 Hz, 1H), 6.87 (d, J=8.5 Hz, 1H), 6.22 (t, J=5.7 Hz, 1H), 4.91 (dd, J=12.1, 5.3 Hz, 1H), 3.25 (td, J=7.1, 5.6 Hz, 2H), 2.96-2.67 (m, 3H), 2.20 (t, J=7.5 Hz, 2H), 2.17-2.09 (m, 1H), 1.70-1.53 (m, 4H), 1.43 (s, 9H), 1.37-1.30 (m, 5H).

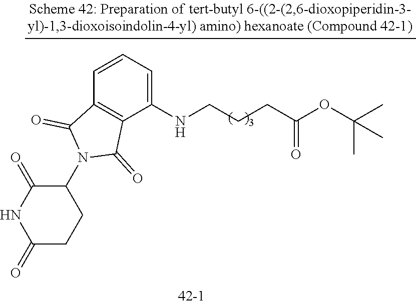

Compound 42-1 was prepared following General Procedure 5 using 4-fluoro-thalidomide (516 mg, 1.9 mmol) and the corresponding tert-butyl ester (350 mg, 1.9 mmol). The volatiles were then removed in vacuo and the crude product was purified using flash column chromatography (SiO₂; EtOAc/Hept=2:3) providing 623 mg of the desired product (75% yield). ¹H NMR (400 MHz, CDCl₃) δ 7.89 (s, 1H), 7.42 (dd, J=8.5, 7.1 Hz, 1H), 7.02 (d, J=7.0 Hz, 1H), 6.81 (d, J=8.5 Hz, 1H), 6.16 (t, J=5.7 Hz, 1H), 4.84 (dd, J=12.1, 5.4 Hz, 1H), 3.20 (td, J=7.1, 5.6 Hz, 2H), 2.89-2.59 (m, 3H), 2.17 (t, J=7.4 Hz, 2H), 2.10-2.04 (m, 1H), 1.68-1.53 (m, 4H), 1.37 (s, 9H).

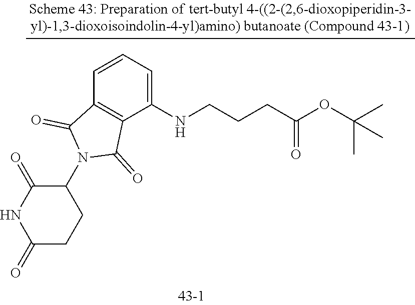 [SMILES: CC(C)(C)OC(=O)CCCNc1cccc2c1C(=O)N(C1CCC(=O)NC1=O)C2=O]

Compound 43-1 was prepared following General Procedure 5 using 4-fluoro-thalidomide (55 mg, 0.2 mmol) and the corresponding tert-butyl ester (34 mg, 0.2 mmol). The volatiles were then removed in vacuo and the crude product was purified using flash column chromatography (SiO₂; EtOAc/Hept=2:3) providing 80 mg of the desired product (96% yield). ¹H NMR (400 MHz, CDCl₃) δ 8.02 (s, 1H), 7.50 (dd, J=8.6, 7.1 Hz, 1H), 7.10 (d, J=7.0 Hz, 1H), 6.93 (d, J=8.5 Hz, 1H), 6.29 (t, J=5.8 Hz, 1H), 4.96-4.84 (m, 1H), 3.33 (q, J=6.6 Hz, 2H), 2.93-2.69 (m, 3H), 2.35 (t, J=7.1 Hz, 2H), 2.17-2.10 (m, 1H), 1.94 (p, J=7.1 Hz, 2H), 1.45 (s, 9H). ¹³C NMR (101 MHz, CDCl₃) δ 172.4, 171.1, 169.6, 168.4, 167.7, 147.0, 136.3, 132.7, 116.8, 111.8, 110.2, 80.8, 49.0, 42.0, 32.7, 31.6, 28.3, 24.8, 22.9. LRMS (ESI) m/z: [M+H]+ calcd for C21H26N3O6; 416.181 found, 416.182.

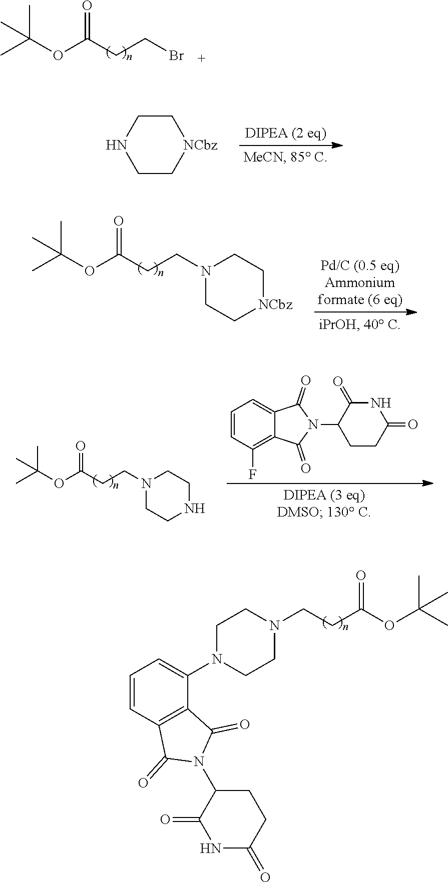

To a stirred solution of corresponding tert-butyl ester (1 eq) in MeCN (0.5 M) was subsequently added 1-Cbz-Piperazine (1 eq) and DIPEA (2 eq). The resulting reaction mixture was stirred at 85° C. (oil bath temperature) until full completion (Monitored by TLC). The volatiles were then removed in vacuo and the residue was extracted into EtOAc (3×). The combined organic layers were dried over MgSO₄, filtered and concentrated under reduced pressure. The product was involved in the next step without further purification.

The piperazine derivative (1 eq) was added to a pre-prepared mixture of iPrOH (0.5 M) and Pd/C (0.5 eq), followed by addition of ammonium formate (6 eq). The resulting reaction mixture was stirred at 40° C. (oil bath temperature) until full completion (Monitored by TLC). The reaction mixture was evaporated and extracted into DCM (3×). The combined organic layers were dried over MgSO₄, filtered and evaporated. The product was involved in the next step without further purification.

To a stirred solution of 4-fluoro-thalidomide (1 eq) in DMSO (0.5 M) was subsequently added piperazine derivative (1 eq) and DIPEA (3 eq). The resulting reaction mixture was stirred at 130° C. (oil bath temperature) until completion (Monitored by TLC).

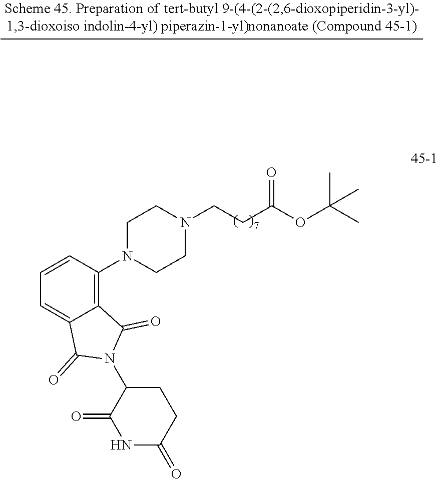

Compound 45-1 was prepared following the General procedure 6 using 4-fluoro-thalidomide (60 mg, 0.2 mmol) and the corresponding tert-butyl ester (55 mg, 0.2 mmol). The volatiles were then removed in vacuo and the crude product was purified using flash column chromatography (SiO₂; EtOAc/MeOH=95:5) providing 68 mg of the desired product (61%). ¹H NMR (400 MHz, DMSO) δ 11.08 (s, 1H), 7.70 (dd, J=8.4, 7.1 Hz, 1H), 7.34 (t, J=8.3 Hz, 2H), 5.09 (dd, J=12.8, 5.4 Hz, 1H), 2.87 (ddd, J=18.2, 13.8, 5.3 Hz, 1H), 2.61 (m, 1H), 2.39-2.27 (m, 2H), 2.17 (t, J=7.3 Hz, 2H), 2.07-1.97 (m, 1H), 1.47 (m, 4H), 1.39 (s, 9H), 1.25 (m, 10H). ¹³C NMR (101 MHz, DMSO) δ 173.3, 172.8, 170.5, 167.5, 166.8, 150.2, 134.1, 116.9, 79.8, 58.3, 53.2, 51.0, 49.3, 40.7, 40.6, 40.4, 35.2, 31.4, 29.3, 29.1, 28.8, 28.2, 27.3, 26.7, 25.1. LRMS (ESI) m/z: [M+H]+ calcd for C30H43N4O6; 555.320 found, 555.318.

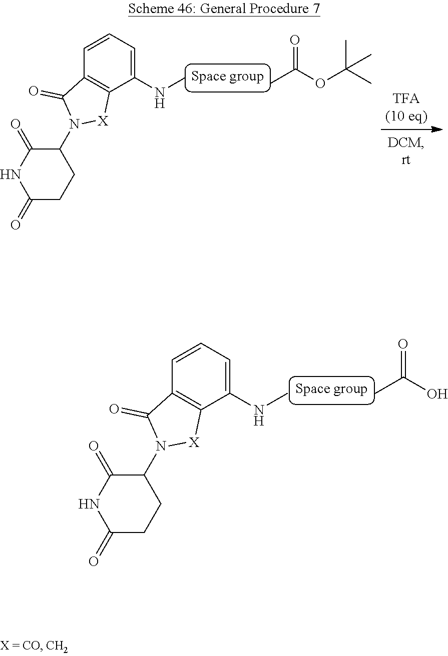

To a stirred solution of corresponding tert-butyl ester (1 eq) in DCM (0.5 M) was added TFA (10 eq). The resulting reaction mixture was stirred at rt until full completion (Monitored by TLC). The volatiles were then removed in vacuo.

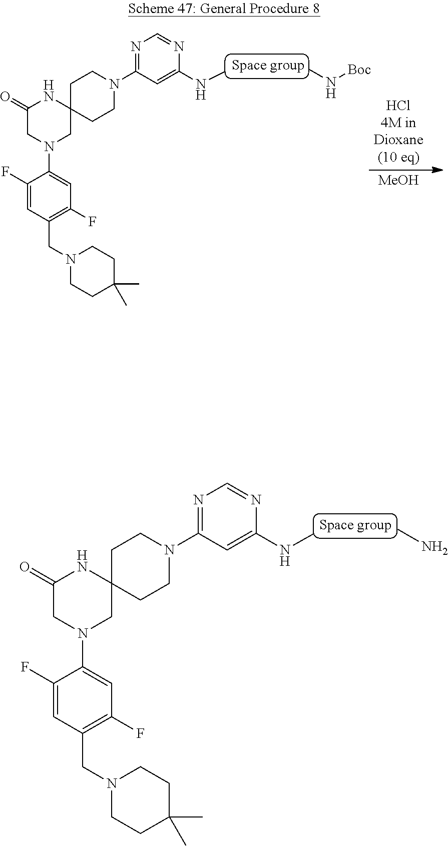

Boc protected amine (1 eq) was dissolved in MeOH (0.25 M), to the stirred solution was then added HCl 4M in dioxane (10 eq). The resulting reaction mixture was stirred at rt until completion (Monitored by TLC) and evaporated.

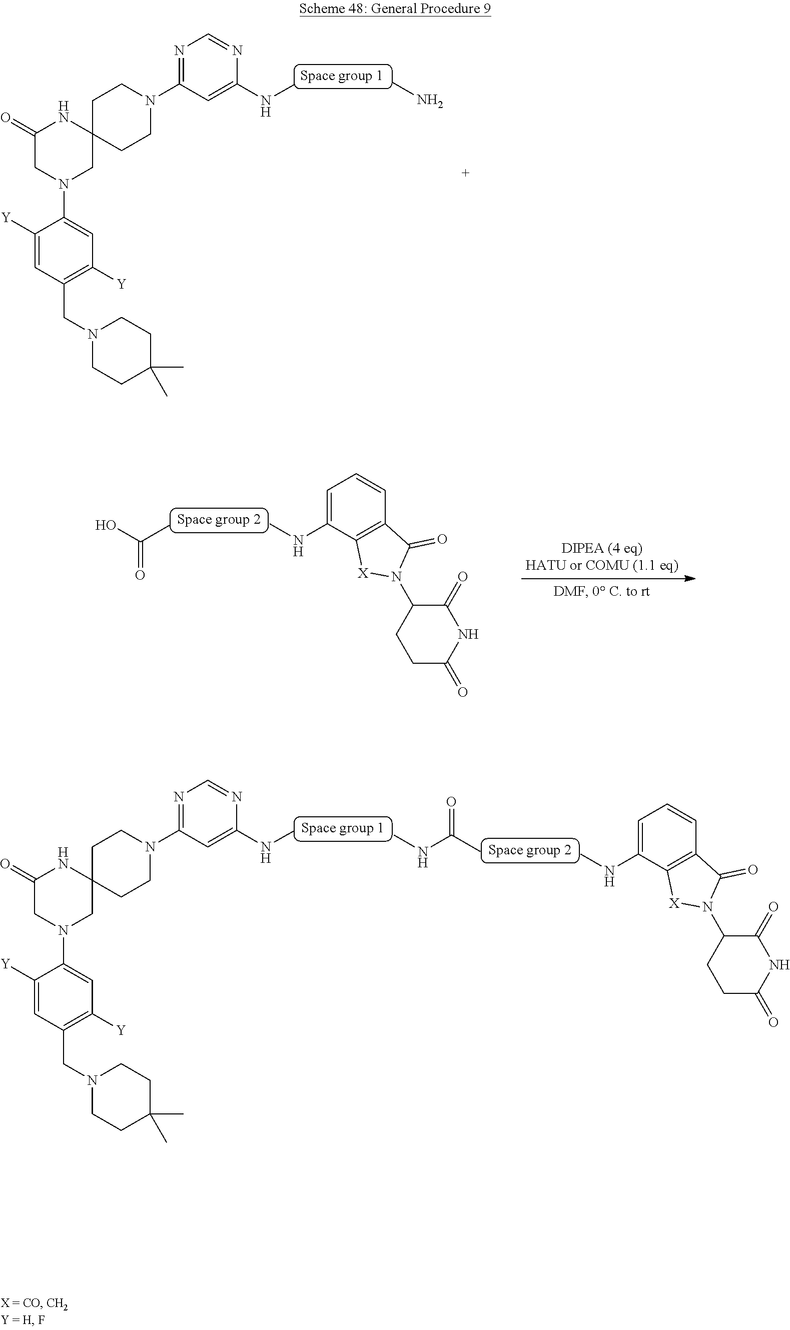

To a cooled solution (water-ice bath) of carboxylic acid (1 eq) in DMF (0.5 M) was added DIPEA (4 eq), and the reaction mixture was stirred at the same temperature for 10 minutes. After addition of HATU or COMU (1.1 eq), the solution was stirred for an additional 30 minutes after which the amine (1 eq) was added. The resulting reaction mixture was stirred at rt until completion (Monitored by TLC), concentrated in vacuo and purified using flash column chromatography.

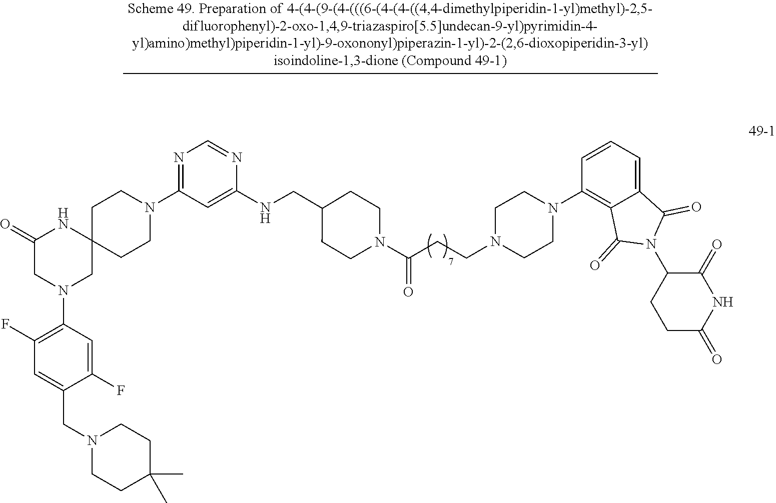

Compound 49-1 was prepared following General Procedure 9 (COMU) using corresponding amine (58 mg, 0.09 mmol) and carboxylic acid (60 mg, 0.09 mmol). The reaction mixture was concentrated in vacuo and the crude product was purified using flash column chromatography (SiO₂; EtOAc/MeOH=from 85:15 to 80:20) providing 65 mg of the desired product (62%). ¹H NMR (400 MHz, MeOD) δ 7.99 (s, 1H), 7.67 (dd, J=8.4, 7.2 Hz, 1H), 7.38 (d, J=7.1 Hz, 1H), 7.31 (d, J=8.4 Hz, 1H), 7.18 (dd, J=13.0, 6.6 Hz, 1H), 6.87 (dd, J=11.2, 7.2 Hz, 1H), 5.69 (s, 1H), 5.09 (dd, J=12.5, 5.5 Hz, 1H), 4.54 (d, J=13.3 Hz, 1H), 4.03-3.96 (m, 1H), 3.92 (m, 2H), 3.72 (s, 2H), 3.62-3.57 (s, 2H), 3.42-3.33 (m, 7H), 3.18 (m, 2H), 3.08 (t, J=12.9 Hz, 1H), 2.86 (ddd, J=17.7, 14.2, 5.1 Hz, 1H), 2.77 (m, 5H), 2.66-2.57 (m, 1H), 2.54 (m, 4H), 2.51-2.44 (m, 2H), 2.39 (td, J=7.4, 3.8 Hz, 2H), 2.15-2.07 (m, 1H), 1.98 (m, 2H), 1.91 (s, 1H), 1.89-1.76 (m, 5H), 1.59 (m, f 5H), 1.44 (t, J=5.6 Hz, 5H), 1.37 (s, 8H), 0.93 (s, 6H). ¹³C NMR (101 MHz, MeOD) δ 173.2, 172.6, 170.2, 168.9, 167.5, 166.7, 163.4, 162.0, 156.9, 150.0, 135.5, 134.1, 123.3, 117.5, 115.1, 58.3, 53.9, 53.2, 52.8, 52.3, 50.1, 49.1, 45.9, 45.7, 41.6, 40.3, 37.6, 36.2, 34.5, 32.8, 30.8, 30.4, 29.5, 29.4, 29.0, 28.9, 28.9, 27.7, 27.1, 25.9, 25.3, 22.3. LRMS (ESI) m/z: [M+H]+ calcd for C58H79F2N12O6; 1077.620 found, 1077.623.

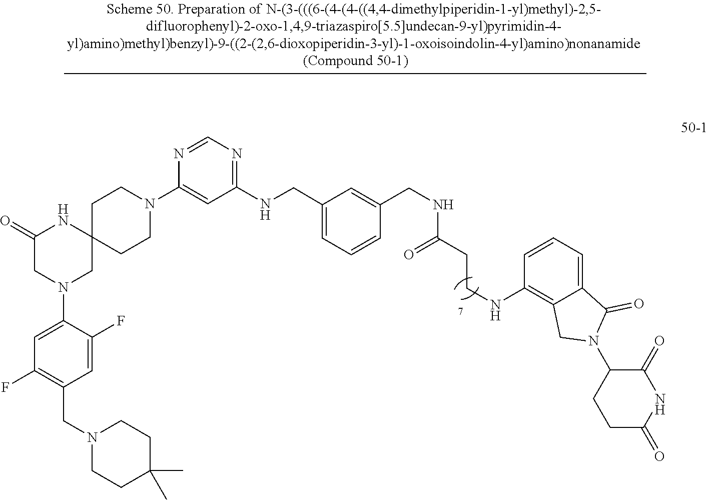

Compound 50-1 was prepared following General Procedure 9 (COMU) using the corresponding amine (25 mg, 0.04 mmol) and carboxylic acid (17 mg, 0.04 mmol). The reaction mixture was concentrated in vacuo and the crude product was purified using flash column chromatography (SiO₂; EtOAc/MeOH=from 90:10 to 80:20) providing 25 mg of the desired product (61%). ¹H NMR (400 MHz, DMSO) δ 11.01 (s, 1H), 8.35 (t, J=6.0 Hz, 1H), 8.18 (s, 1H), 7.98 (s, 1H), 7.31-7.20 (m, 3H), 7.20-7.12 (m, 3H), 7.08 (d, J=7.5 Hz, 1H), 6.97-6.86 (m, 2H), 6.71 (d, J=8.0 Hz, 1H), 5.69 (s, 1H), 5.60 (t, J=5.6 Hz, 1H), 5.10 (dd, J=13.3, 5.1 Hz, 1H), 4.41 (d, J=6.2 Hz, 2H), 4.22 (s, 2H), 4.18 (dd, J=45.9, 17.1 Hz, 2H), 3.83 (d, J=13.3 Hz, 2H), 3.58 (s, 2H), 3.47-3.37 (m, 4H), 3.25 (s, 2H), 3.09 (q, J=6.6 Hz, 2H), 2.92 (ddd, J=18.1, 13.5, 5.4 Hz, 1H), 2.67-2.57 (m, 1H), 2.40-2.22 (m, 4H), 2.11 (t, J=7.4 Hz, 2H), 2.06-1.97 (m, 1H), 1.81-1.72 (m, 2H), 1.69-1.60 (m, 1H), 1.60-1.44 (m, 3H), 1.38-1.20 (m, 14H), 0.87 (s, 6H). ¹³C NMR (101 MHz, DMSO) δ 172.9, 172.1, 171.3, 168.9, 166.5, 163.2, 161.6, 157.4, 143.8, 139.8, 132.0, 129.2, 128.1, 126.5, 126.0, 125.5, 125.5, 111.7, 109.8, 69.8, 59.8, 54.7, 52.8, 52.6, 51.5, 49.0, 45.8, 43.6, 42.7, 41.9, 38.1, 35.3, 34.6, 31.2, 28.9, 28.8, 28.7, 28.5, 28.1, 26.7, 25.3, 22.8, 21.1, 20.8, 14.1. LCMS (ESI) m/z: [M+H]+ calcd for C56H72F2N11O5; 1016.568 found, 1016.566.

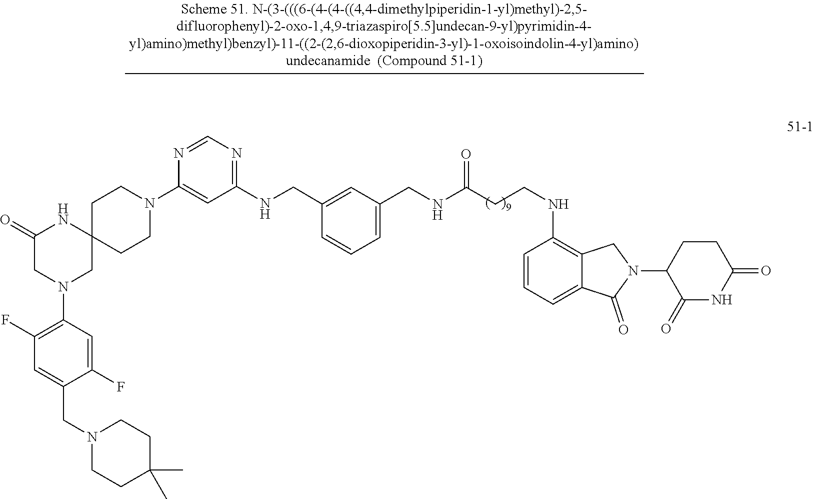

Compound 51-1 was prepared following General Procedure 9 (COMU) using the corresponding amine (18.6 mg, 0.04 mmol) and carboxylic acid (26 mg, 0.04 mmol). The reaction mixture was concentrated in vacuo and the crude product was purified using flash column chromatography (SiO₂; DCM/MeOH=from 90:10 to 80:20) providing 32 mg of the desired product (73%). ¹H NMR (400 MHz, MeOD) δ 8.06 (s, 1H), 7.47-7.38 (m, 1H), 7.33-7.25 (m, 3H), 7.22 (d, J=7.8 Hz, 1H), 7.17 (d, J=7.5 Hz, 1H), 7.03 (d, J=7.5 Hz, 1H), 6.98 (dd, J=11.4, 7.2 Hz, 1H), 6.79 (d, J=8.0 Hz, 1H), 5.69 (s, 1H), 5.13 (dd, J=13.3, 5.1 Hz, 1H), 4.48 (s, 2H), 4.34 (s, 2H), 4.30-4.26 (m, 3H), 3.94-3.86 (m, 2H), 3.76 (s, 2H), 3.75-3.69 (m, 2H), 3.64 (s, 1H), 3.57 (dd, J=10.8, 8.1 Hz, 2H), 3.49-3.43 (m, 1H), 3.41 (s, 2H), 3.26-3.16 (m, 6H), 2.90 (ddd, J=18.5, 13.3, 5.1 Hz, 1H), 2.81-2.73 (m, 1H), 2.46 (qd, J=13.1, 4.7 Hz, 1H), 2.23-2.14 (m, 3H), 1.97-1.89 (m, 2H), 1.78 (m, 2H), 1.70-1.57 (m, 7H), 1.39-1.36 (m, 16H), 1.05 (s, 4H). ¹³C NMR (101 MHz, MeOD) δ 174.8, 173.3, 171.1, 171.0, 168.4, 161.2, 143.8, 141.1, 139.4, 131.6, 129.2, 128.5, 126.6, 126.1, 125.9, 125.8, 112.4, 110.4, 54.4, 53.0, 52.2, 51.9, 48.6, 46.1, 44.6, 43.1, 42.5, 42.4, 40.5, 35.7, 35.1, 34.3, 31.0, 29.4, 29.2, 29.1, 28.9, 28.8, 28.8, 27.3, 26.8, 25.6, 22.9, 17.3, 15.9, 11.8. LRMS (ESI) m/z: [M+H]+ calcd for C58H76F2N11O5; 1044.600 found, 1044.600.

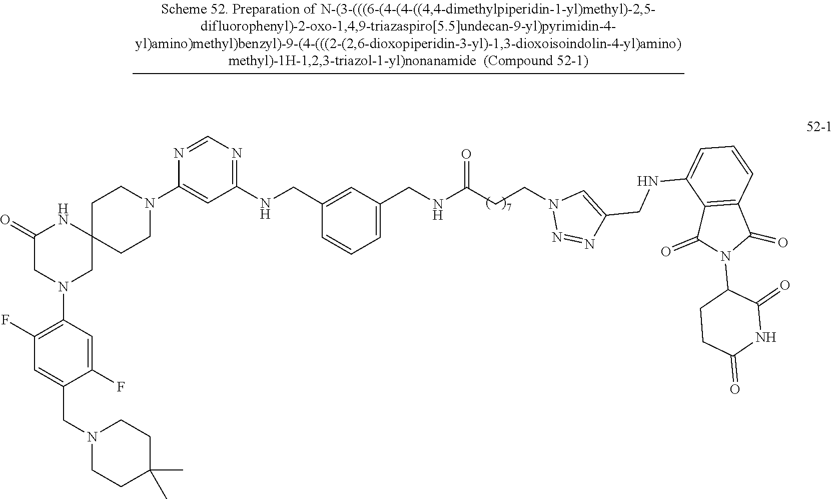

Compound 52-1 was prepared following General Procedure 9 (COMU) using the corresponding amine (18.2 mg, 0.03 mmol) and carboxylic acid (15 mg, 0.03 mmol). The reaction mixture was concentrated in vacuo and the crude product was purified using flash column chromatography (SiO₂; DCM/MeOH=from 90:10 to 80:20) providing 10 mg of the desired product (31%). ¹H NMR (500 MHz, CDCl₃) δ 10.24 (bs, 1H), 8.16 (s, 1H), 7.51-7.41 (m, 2H), 7.29 (m, 1H), 7.22-7.13 (m, 3H), 7.11 (d, J=7.1 Hz, 1H), 6.98 (d, J=8.5 Hz, 1H), 6.81 (s, 1H), 6.67 (t, J=6.0 Hz, 1H), 6.58 (dd, J=10.9, 7.0 Hz, 1H), 6.03 (t, J=6.0 Hz, 1H), 5.37 (s, 1H), 4.89 (dd, J=12.2, 5.4 Hz, 1H), 4.62 (d, J=5.9 Hz, 2H), 4.41 (dd, J=11.9, 5.8 Hz, 3H), 4.31 (td, J=6.9, 2.5 Hz, 2H), 3.68 (m, 4H), 3.50 (m, 2H), 3.28 (s, 2H), 2.89-2.83 (m, 1H), 2.75 (m, 2H), 2.56 (bs, 2H), 2.12 (t, J=7.1, 6.5 Hz, 2H), 1.87 (m, 4H), 1.75 (m, 3H), 1.57 (t, J=7.8, 7.1 Hz, 2H), 1.45 (m, 2H), 1.26 (m, 16H), 0.94 (s, 4H), 0.88 (t, J=6.7 Hz, 2H). ¹³C NMR (101 MHz, CDCl₃) δ 173.2, 171.8, 169.4, 169.2, 167.8, 167.6, 146.2, 139.3, 138.6, 136.2, 132.4, 129.0, 126.7, 126.1, 125.9, 121.5, 117.2, 112.3, 110.8, 53.4, 52.9, 50.4, 49.6, 49.0, 45.5, 43.2, 40.4, 38.8, 36.6, 35.0, 31.9, 31.5, 30.0, 29.7, 29.33, 29.0, 29.0, 29.0, 28.6, 28.3, 27.2, 26.2, 25.6, 22.8, 22.7, 14.1. LRMS (ESI) m/z: [M+H]+ calcd for C59H73F2N14O6; 1111.580 found, 1111.581.

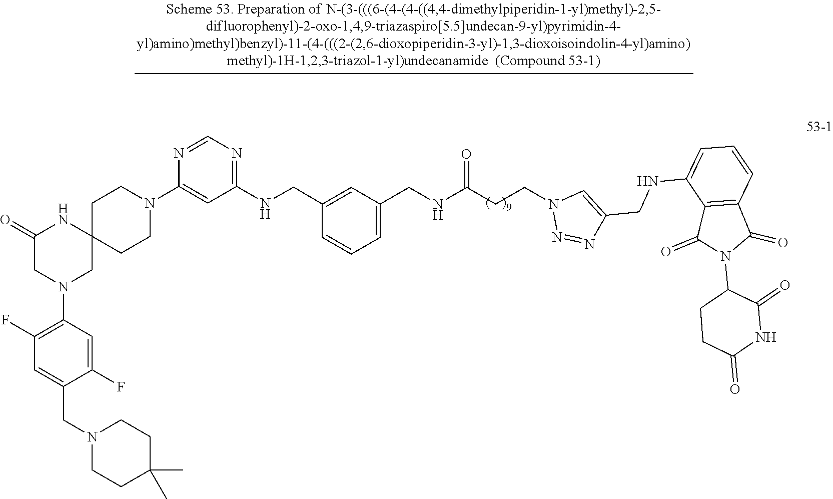

Compound 27 was prepared following General Procedure 9 (COMU) using the corresponding amine (17 mg, 0.03 mmol) and carboxylic acid (15 mg, 0.03 mmol). The reaction mixture was concentrated in vacuo and the crude product was purified using flash column chromatography (SiO₂; DCM/MeOH=from 90:10 to 80:20) providing 12 mg of the desired product (37%). ¹H NMR (400 MHz, CDCl₃) δ 10.08 (bs, 1H), 8.16 (s, 1H), 7.50-7.43 (m, 2H), 7.21 (s, 1H), 7.16 (m, 2H), 7.10 (d, J=7.2 Hz, 1H), 6.97 (d, J=8.5 Hz, 1H), 6.83 (s, 1H), 6.68 (t, J=5.9 Hz, 1H), 6.58 (dd, J=10.9, 7.1 Hz, 1H), 6.07 (s, 1H), 5.93 (bs, 1H), 5.40-5.32 (m, 2H), 4.90 (dd, J=11.9, 5.3 Hz, 1H), 4.62 (d, J=5.9 Hz, 2H), 4.42 (m, 3H), 4.31 (t, J=7.0 Hz, 2H), 3.71 (s, 2H), 3.65 (m, 2H), 3.59-3.45 (m, 4H), 3.27 (s, 2H), 2.90-2.69 (m, 3H), 2.48 (bs, 2H), 2.26-2.07 (m, 3H), 2.02 (m, 2H), 1.93-1.83 (m, 4H), 1.80-1.71 (m, 2H), 1.58 (m, 4H), 1.42 (m, 4H), 1.28-1.25 (m, 12H), 0.92 (s, 6H). ¹³C NMR (101 MHz, CDCl₃) δ 173.2, 171.8, 169.4, 169.2, 167.8, 167.6, 146.2, 139.3, 138.6, 136.2, 132.4, 129.0, 126.7, 126.1, 125.9, 121.5, 117.2, 112.3, 110.8, 53.4, 52.9, 50.4, 49.6, 49.0, 45.5, 43.2, 40.4, 38.8, 36.6, 35.0, 31.9, 31.5, 30.0, 29.7, 29.3, 29.0, 29.0, 29.0, 28.6, 28.3, 27.2, 26.2, 25.6, 22.8, 22.7, 14.1. LRMS (ESI) m/z: [M+H]+ calcd for C61H77F2N14O6; 1139.610 found, 1139.608.

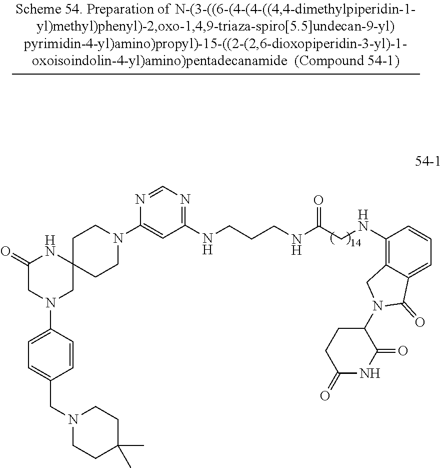

Compound 54-1 was prepared following General Procedure 9 (COMU) using the corresponding amine (117 mg, 0.224 mmol) and corresponding carboxylic acid (112 mg, 0.224 mmol). The reaction mixture was concentrated in vacuo and the crude product was purified using flash column chromatography (SiO₂; DCM/MeOH=from 90:10 to 80:20) providing the desired product (120 mg, 53%). ¹H NMR (400 MHz, MeOD) δ 8.01 (s, 1H), 7.35-7.31 (m, 2H), 7.21 (t, J=7.8 Hz, 1H), 7.00-6.87 (m, 3H), 6.71 (d, J=8.0 Hz, 1H), 5.71 (s, 1H), 5.04 (dd, J=13.3, 5.1 Hz, 1H), 4.19 (d, J=6.4 Hz, 2H), 4.13 (d, J=7.4 Hz, 2H), 3.78 (d, J=3.7 Hz, 4H), 3.70-3.60 (m, 2H), 3.45 (s, 2H), 3.19-3.14 (m, 3H), 3.10 (t, J=7.2 Hz, 2H), 3.05-2.94 (m, 2H), 2.81 (ddd, J=18.4, 13.4, 5.4 Hz, 1H), 2.68 (ddd, J=17.6, 4.7, 2.5 Hz, 1H), 2.38 (qd, J=13.2, 4.7 Hz, 1H), 2.13-2.04 (m, 3H), 1.83 (td, J=8.1, 6.6, 3.4 Hz, 2H), 1.73 (ddd, J=20.3, 9.7, 5.4 Hz, 3H), 1.53 (tt, J=14.5, 6.5 Hz, 9H), 1.35-1.15 (m, 26H), 0.95 (d, J=14.2 Hz, 7H). ¹³C NMR (101 MHz, MeOD) δ 175.2, 173.3, 171.1, 171.0, 150.2, 143.8, 132.3, 131.6, 129.2, 126.6, 119.0, 114.3, 112.4, 110.4, 59.6, 52.6, 52.4, 52.2, 50.4, 46.1, 43.1, 40.6, 38.7, 36.2, 35.8, 35.2, 34.1, 31.0, 29.2, 29.2, 29.1, 29.0, 28.9, 28.8, 28.4, 27.4, 26.8, 25.6, 22.9, 22.1. LRMS (ESI) m/z: [M+H]+ calcd for C57H84N11O5; 1002.670 found, 1002.664.

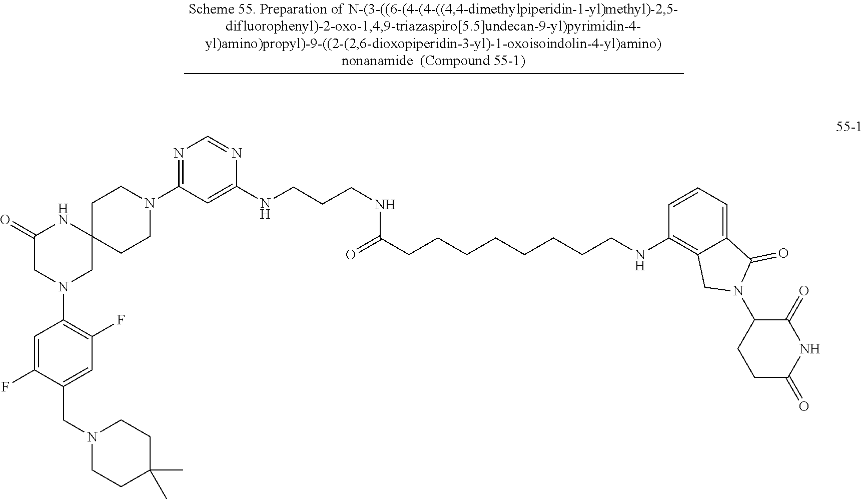 [SMILES: CC1(C)CCN(Cc2cc(F)c(N3CC(=O)NC4(CCN(c5cc(NCCCNC(=O)CCCCCCCCNc6cccc7c6CN(C6CCC(=O)NC6=O)C7=O)ncn5)CC4)C3)cc2F)CC1]

Compound 55-1 was prepared following General Procedure 9 (COMU) using the corresponding amine (85 mg, 0.14 mmol) and carboxylic acid (60 mg, 0.24 mmol). The reaction mixture was concentrated in vacuo and the crude product was purified using flash column chromatography (SiO₂; EtOAc/MeOH=from 90:10 to 80:20) providing 100 mg of the desired product (72%). ¹H NMR (400 MHz, CDCl₃) δ 8.14 (s, 1H), 7.32 (t, J=7.7 Hz, 1H), 7.20 (d, J=7.5 Hz, 1H), 7.11 (dd, J=12.9, 6.6 Hz, 1H), 7.02 (s, 1H), 6.76 (d, J=8.1 Hz, 1H), 6.57 (dd, J=10.9, 7.1 Hz, 1H), 6.36 (s, 1H), 5.74 (s, 1H), 5.43 (s, 1H), 5.20 (dd, J=13.2, 5.2 Hz, 1H), 4.20 (dd, J=65.9, 15.8 Hz, 2H), 3.71 (d, J=7.1 Hz, 4H), 3.60-3.53 (m, 2H), 3.50 (s, 2H), 3.29 (q, J=6.9 Hz, 4H), 3.25 (s, 2H), 3.18 (q, J=7.1, 6.6 Hz, 2H), 2.85-2.75 (m, 2H), 2.47-2.38 (m, 4H), 2.33-2.19 (m, 3H), 2.19-2.07 (m, 4H), 1.98-1.86 (m, 2H), 1.84-1.69 (m, 2H), 1.66-1.53 (m, 4H), 1.44-1.36 (m, 5H), 1.35-1.21 (m, 7H), 0.90 (s, 6H). ¹³C NMR (101 MHz, CDCl₃) δ 173.8, 172.2, 171.1, 170.3, 168.3, 163.1, 162.3, 157.4, 156.2, 152.0, 149.9, 143.4, 137.6, 132.0, 129.8, 126.3, 118.6, 113.3, 112.5, 105.9, 105.7, 56.6, 56.5, 54.7, 53.5, 53.1, 51.9, 51.0, 49.8, 45.2, 43.7, 40.3, 38.9, 38.6, 38.3, 38.3, 36.9, 36.5, 35.3, 31.8, 29.8, 29.3, 29.2, 29.0, 28.9, 28.5, 26.8, 25.7, 23.7. LCMS (ESI) m/z: [M+H]+ calcd for C51H70F₂N11O5; 954.552 found, 954.552.

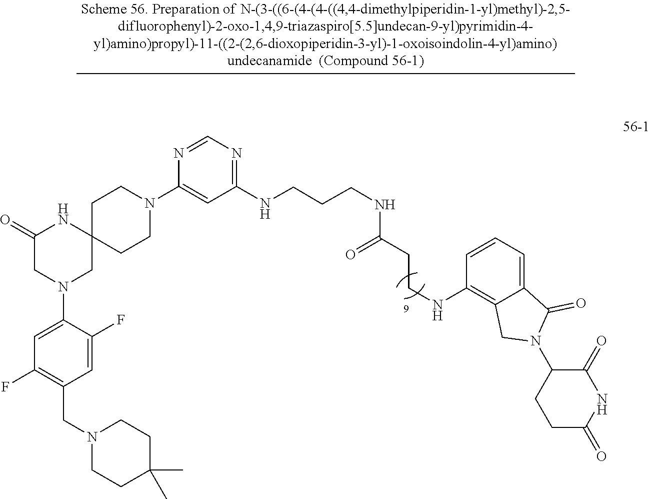

Compound 56-1 was prepared following General Procedure 9 (COMU) using the corresponding amine (40 mg, 0.07 mmol) and carboxylic acid (30 mg, 0.07 mmol). The reaction mixture was concentrated in vacuo and the crude product was purified using flash column chromatography (SiO₂; EtOAc/MeOH=from 90:10 to 80:20) providing 37 mg of the desired product (56%). ¹H NMR (400 MHz, DMSO) δ 11.00 (s, 1H), 10.87 (s, 1H), 8.36 (s, 1H), 8.29 (s, 1H), 7.99 (t, J=5.5 Hz, 1H), 7.73 (dd, J=13.4, 6.8 Hz, 1H), 7.27 (t, J=7.7 Hz, 1H), 7.05 (dd, J=11.7, 7.4 Hz, 1H), 6.91 (d, J=7.4 Hz, 1H), 6.72 (d, J=8.0 Hz, 1H), 5.94 (s, 1H), 5.10 (dd, J=13.2, 5.1 Hz, 1H), 4.24 (d, J=4.5 Hz, 2H), 4.18 (dd, J=46.2, 17.3 Hz, 2H), 3.69 (s, 2H), 3.40-3.33 (m, 3H), 3.30 (d, J=6.2 Hz, 2H), 3.21-3.01 (m, 8H), 2.92 (ddd, J=18.0, 13.4, 5.2 Hz, 1H), 2.68-2.57 (m, 1H), 2.29 (qd, J=13.4, 4.9 Hz, 1H), 2.13-1.97 (m, 3H), 1.91-1.70 (m, 5H), 1.66 (t, J=6.8 Hz, 2H), 1.60-1.51 (m, 2H), 1.51-1.39 (m, 4H), 1.38-1.16 (m, 17H), 0.99 (s, 3H), 0.93 (s, 3H). ¹³C NMR (101 MHz, DMSO) δ 173.4, 172.8, 171.7, 169.4, 166.9, 158.8, 158.5, 144.2, 141.0, 132.5, 129.7, 127.0, 118.9, 116.0, 112.2, 110.4, 53.3, 52.7, 52.0, 48.2, 46.3, 43.2, 36.4, 35.9, 35.1, 31.7, 31.5, 29.5, 29.4, 29.4, 29.3, 29.2, 29.0, 28.0, 27.1, 25.8, 23.5, 23.3. LCMS (ESI) m/z: [M+H]+ calcd for C53H74F2N11O5; 982.583 found, 982.584.

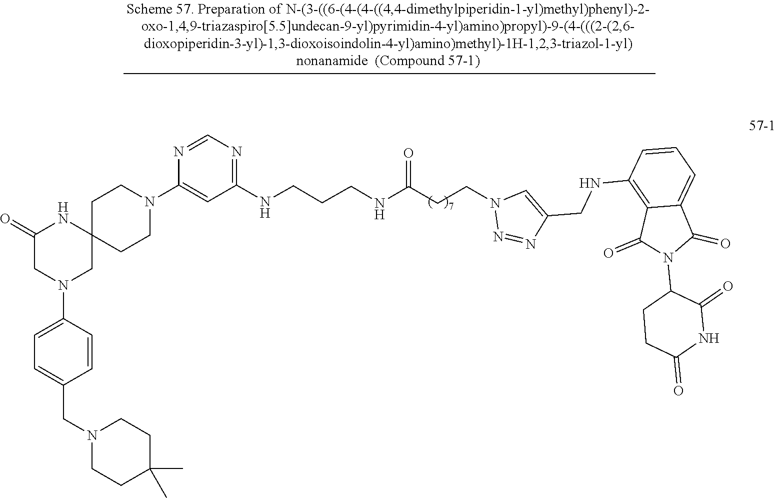

Compound 57-1 was prepared following General Procedure 9 (HATU) using the corresponding amine (67 mg, 0.13 mmol) and corresponding carboxylic acid (66 mg, 0.13 mmol). The reaction mixture was concentrated in vacuo and the crude product was purified using flash column chromatography (SiO₂; DCM/MeOH=from 90:10 to 80:20) providing 100 mg of the desired product (76%). ¹H NMR (400 MHz, MeOD) δ 7.88 (d, J=0.8 Hz, 1H), 7.81 (s, 1H), 7.41 (dd, J=8.5, 7.1 Hz, 1H), 7.28 (d, J=8.7 Hz, 2H), 6.97 (t, J=7.9 Hz, 2H), 6.92-6.88 (m, 2H), 5.58 (d, J=1.0 Hz, 1H), 4.94 (dd, J=12.5, 5.4 Hz, 1H), 4.52 (s, 2H), 4.24 (t, J=7.0 Hz, 2H), 4.01 (s, 2H), 3.76 (s, 2H), 3.69 (q, J=5.6, 3.7 Hz, 2H), 3.49-3.43 (m, 2H), 3.42 (s, 2H), 3.25 (s, 1H), 3.19-3.11 (m, 4H), 2.98 (s, 3H), 2.76-2.67 (m, 1H), 2.64 (dd, J=4.3, 2.4 Hz, 1H), 2.62-2.56 (m, 1H), 2.05 (d, J=7.5 Hz, 2H), 1.79-1.64 (m, 8H), 1.48 (dt, J=19.1, 6.5 Hz, 7H), 1.27 (dd, J=7.2, 4.5 Hz, 4H), 1.16 (d, J=3.3 Hz, 8H), 0.93 (s, 6H). ¹³C NMR (101 MHz, MeOD) δ 175.0, 173.2, 170.2, 169.2, 167.8, 163.2, 162.0, 156.9, 150.0, 146.0, 145.1, 135.8, 132.5, 132.1, 122.7, 117.0, 114.2, 111.1, 110.6, 60.0, 54.4, 52.9, 52.5, 50.5, 49.9, 40.2, 38.2, 37.5, 36.5, 35.7, 35.5, 34.1, 30.8, 29.7, 28.8, 28.7, 28.5, 28.3, 27.5, 25.8, 25.5, 22.4, 11.8. LRMS (ESI) m/z: [M+H]+ calcd for C54H73N14O6; 1013.580 found, 1013.584.

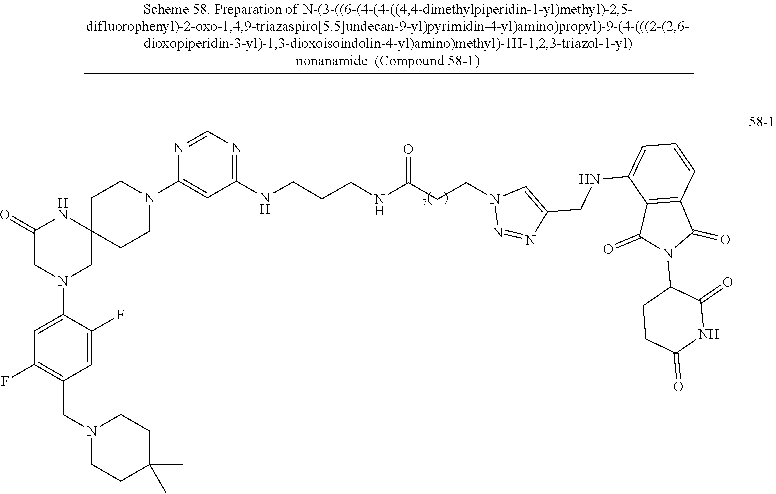

Compound 58-1 was prepared following General Procedure 9 (COMU) using the corresponding amine (34.9 mg, 0.063 mmol) and carboxylic acid (32.0 mg, 0.063 mmol). The reaction mixture was concentrated in vacuo and the crude product was purified using flash column chromatography (SiO₂; DCM/MeOH=from 100:5 to 100:20) providing 15 mg of the desired product (22.3%). ¹H NMR (400 MHz, CDCl₃) δ 10.38 (bs, 1H), 8.14 (s, 1H), 7.47 (dd, J=8.5, 7.1 Hz, 1H), 7.13 (dd, J=12.8, 6.7 Hz, 1H), 7.11 (d, J=7.1 Hz, 1H), 6.98 (d, J=8.5 Hz, 1H), 6.90 (s, 1H), 6.70 (t, J=5.9 Hz, 1H), 6.58 (dd, J=10.9, 7.2 Hz, 1H), 6.28 (bs, 1H), 5.79 (bs, 1H), 5.44 (s, 1H), 4.91 (dd, J=12.1, 5.4 Hz, 1H), 4.63 (d, J=5.9 Hz, 2H), 4.32 (t, J=7.0 Hz, 2H), 3.75-3.49 (m, 8H), 2.87-2.81 (m, 3H), 2.49 (bs, 4H), 2.17-2.05 (m, 3H), 1.95-1.91 (m, 8H), 1.60-1.51 (m, 2H), 1.45-1.38 (m, 4H), 1.28-1.17 (m, 10H), 0.92 (s, 6H). ¹³C NMR (101 MHz, CDCl₃) δ 207.1, 173.8, 171.8, 169.8, 169.6, 168.1, 167.7, 162.5, 162.3, 158.7, 157.2, 152.3, 146.3, 145.2, 136.4, 132.5, 121.8, 118.9, 117.3, 112.4, 110.9, 105.9, 105.6, 63.9, 56.7, 54.5, 53.5, 53.0, 50.5, 49.7, 49.1, 40.3, 38.9, 38.3, 36.8, 36.5, 35.4, 32.1, 31.6, 31.1, 30.1, 29.8, 29.5, 29.3, 28.8, 28.4, 28.4, 26.2, 25.6, 23.0. LRMS (ESI) m/z: [M+H]+ calcd for C54H71F2N14O6; 1049.564 found, 1049.565.

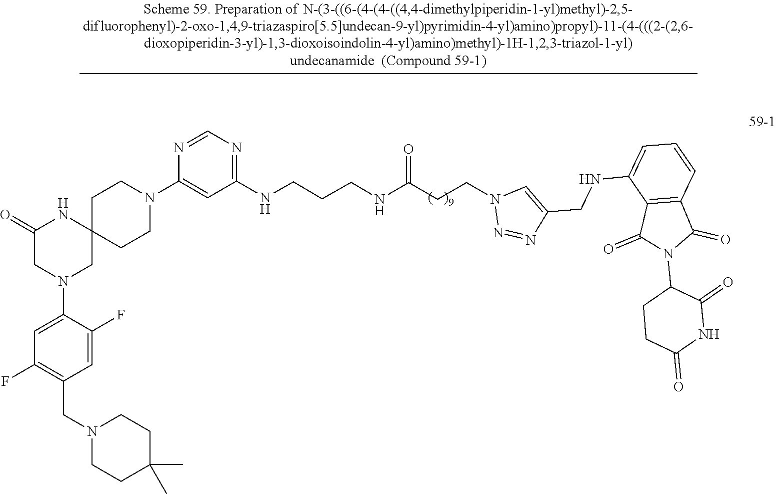

Compound 59-1 was prepared following General Procedure 9 (COMU) using the corresponding amine (33.08 mg, 0.059 mmol) and carboxylic acid (32.0 mg, 0.059 mmol). The reaction mixture was concentrated in vacuo and the crude product was purified using flash column chromatography (SiO₂; DCM/MeOH=from 100:10 to 100:20) providing 14 mg of the desired product (21%). ¹H NMR (400 MHz, CDCl₃) δ 10.38 (bs, 1H), 8.14 (s, 1H), 7.47 (dd, J=8.5, 7.1 Hz, 1H), 7.13 (dd, J=12.8, 6.7 Hz, 1H), 7.11 (d, J=7.1 Hz, 1H), 7.05 (s, 1H), 6.98 (d, J=8.5 Hz, 1H), 6.70 (t, J=5.9 Hz, 1H), 6.58 (dd, J=10.9, 7.2 Hz, 1H), 6.28 (bs, 1H), 5.79 (bs, 1H), 5.44 (s, 1H), 4.91 (dd, J=12.1, 5.4 Hz, 1H), 4.63 (d, J=5.9 Hz, 2H), 4.32 (t, J=7.0 Hz, 2H), 3.75-3.49 (m, 8H), 2.87-2.81 (m, 3H), 2.49 (bs, 4H), 2.17-2.05 (m, 3H), 1.95-1.91 (m, 8H), 1.60-1.51 (m, 2H), 1.45-1.38 (m, 4H), 1.28-1.17 (m, 14H), 0.92 (s, 6H). ¹³C NMR (101 MHz, CDCl3) δ 207.1, 173.9, 171.9, 169.5, 168.2, 167.7, 162.9, 162.3, 158.7, 157.2, 156.3, 152.3, 149.9, 146.3, 145.1, 137.8, 136.3, 132.5, 121.7, 118.8, 118.6, 117.3, 112.4, 110.9, 105.9, 105.6, 63.9, 56.6, 54.6, 53.5, 53.0, 50.6, 49.7, 49.1, 40.3, 38.9, 38.4, 36.9, 36.5, 35.3, 31.6, 31.1, 30.2, 29.8, 29.5, 29.4, 29.1, 29.1, 29.1, 28.7, 28.4, 26.3, 25.8, 22.9. LRMS (ESI) m/z: [M+H]+ calcd for C56H75F2N14O6; 1077.596 found, 1077.593.

Synthesis schemes from literature incorporated by reference herein:

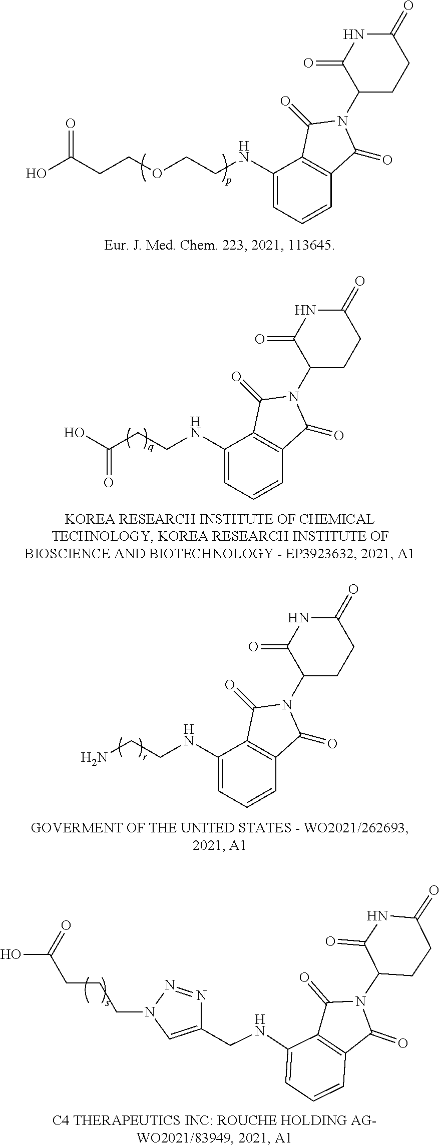

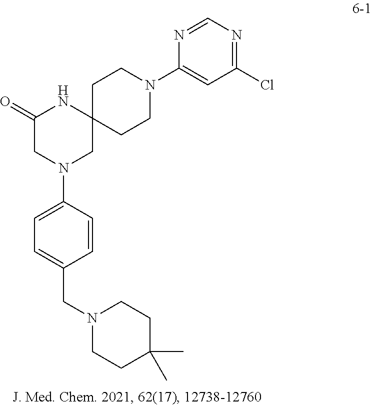 [SMILES: CC1(C)CCN(Cc2ccc(N3CC(=O)NC4(CCN(c5cc(Cl)ncn5)CC4)C3)cc2)CC1]

Also included by reference herein are references Front. Chem. 2021, 9, 707317 (e.g., see schemes 1-20 and FIG. 3) and Org. Biomol. Chem., 2021, 19, 166-170 (e.g., see click chemistry procedures, and methods of making the triazole in Formula J) for general procedures and methods of making starting materials.

### tert-Butyl 4-(aminomethyl)-4-(((benzyloxy)carbonyl)amino)piperidine-1-carboxylate (32)

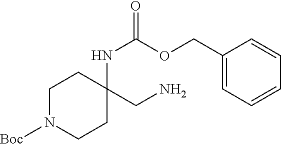 [SMILES: CC(C)(C)OC(=O)N1CCC(CN)(NC(=O)OCc2ccccc2)CC1]

To a stirred solution of MeNO₂ (1.3 equiv. 130 mmol, 7 mL) in NH₃ (53 mL, 7 N in MeOH), tert-butyl 4-oxopiperidine-1-carboxylate (20 g, 100 mmol) was added portionwise. The reaction mixture was stirred at 25° C. for 17 h and concentrated under reduced pressure. The crude residue was diluted with DCM and water. The two phases were separated and the aqueous layer was extracted two times with DCM. The combined organic layers were dried over MgSO₄, filtered and concentrated under reduced pressure to afford the desired product, which was engaged in the next step without further purification.

To a stirred solution of the corresponding amine (100 mmol) in dichloromethane (130 mL), a solution of K₂CO₃ (2 equiv., 200 mmol, 27.6 g) in water (130 mL) was added. The reaction mixture was cooled to 0° C. and CBzCl (1.1 equiv., 110 mmol, 15.6 mL) was added dropwise. The reaction mixture was stirred at 25° C. for 17 h and the two phases were separated. The aqueous layer was extracted two times with DCM. The combined organic layers were washed once with brine, dried over MgSO₄, filtered and concentrated reduced pressure to afford the desired product, which was engaged in the next step without further purification.

To a stirred solution of the corresponding nitroalkane (100 mmol) in dry MeOH (450 mL), under a nitrogen atmosphere, at 0° C., NiCl₂·6H₂O (1 equiv., 100 mmol, 27.3 g) was added, followed by NaBH₄ (5 equiv., 500 mmol, 18.9 g) portionwise to avoid strong H₂ evolution. Caution when adding NaBH₄, the reaction is highly exothermic and produce hydrogen gas. The reaction mixture was stirred at 25° C. for 1 h and quenched by adding saturated aqueous NaHCO₃ solution. The mixture was filtered through a pad of Celite, the filtrate was concentrated under reduced pressure and the obtained residue was diluted with water. The aqueous layer was extracted three times with DCM and the combined organic layers were washed once with brine, dried over MgSO₄, filtered and concentrated under reduced pressure. The crude residue was purified by flash column chromatography (DCM/MeOH/NH₄OH=100:0:0 to 100:3:0 to 100:3:1 to 100:5:1 to 100:10:1 to 100:15:1) to afford the desired product as a white solid (11.7 g, 32% yield over three steps). LRMS (ESI) m/z calcd for [C₁₉H₃₀N₃O₄]⁺: 364.2 found: 364.3

### tert-Butyl 2-oxo-1,4,9-triazaspiro[5.5]undecane-9-carboxylate (33)

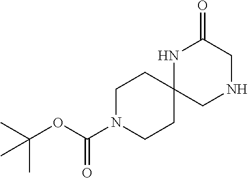 [SMILES: CC(C)(C)OC(=O)N1CCC2(CC1)CNCC(=O)N2]

To a stirred solution of tert-butyl 4-(aminomethyl)-4-(((benzyloxy)carbonyl)amino)piperidine-1-carboxylate (14.5 g, 40 mmol) in DCM (133 mL) at 0° C., Et₃N (0.8 equiv., 32 mmol, 4.4 mL) and ethyl 2-bromoacetate (0.7 equiv., 28 mmol, 3.1 mL) were added. The reaction mixture was stirred at 25° C. for 2 h and diluted with saturated aqueous NaHCO₃ solution. The aqueous layer was extracted three times with EtOAc and the combined organic layers were washed once with brine, dried over MgSO₄, filtered and concentrated under reduced pressure, to afford the impure desired product, which was engaged in the next step without further purification.

To a stirred solution of the corresponding Cbz protected amine (40 mmol) in iPrOH (400 mL), Pd/C (5 mol %, 2 mmol, 2.1 g, 10% wt) and ammonium formate (6 equiv., 240 mmol, 15 g) were added portionwise. The reaction mixture was stirred at 80° C. for 4 h, cooled to 25° C., filtered through a pad of celite and concentrated under reduced pressure. The obtained residue was dissolved in DCM, the organic layer was washed once with water, once with brine, dried over MgSO₄, filtered and concentrated under reduced pressure. The crude residue was purified by flash column chromatography (DCM/MeOH=100:5 to 100:8 to 100:10 to 100:15) to afford the desired product as a white solid (4.14 g, 55% yield over two steps). LRMS (ESI) m/z calcd for C₂₆H₄₇N₆O₆⁺=[2M+H]⁺: 539.4 found: 539.4

### N-Benzyl-6-(4-(6-((4,4-dimethylpiperidin-1-yl)methyl)pyridin-3-yl)-1-oxa-4,9-diazaspiro[5.5]undecan-9-yl)pyrimidin-4-amine (7)

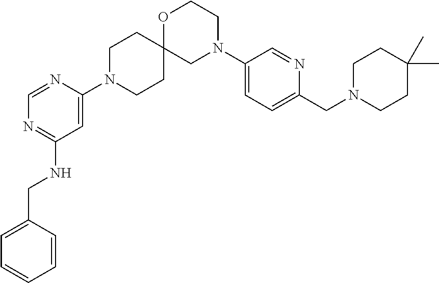 [SMILES: CC1(C)CCN(Cc2ccc(N3CCOC4(CCN(c5cc(NCc6ccccc6)ncn5)CC4)C3)cn2)CC1]

The corresponding Boc protected amine was obtained following the general procedure for Buchwald-Hartwig coupling (chromatography: EtOAc/heptane=7:3 to 9:1 to EtOAc/MeOH=100:0 to 100:1 to 100:5). The impure desired product was engaged in the next step without further purification.

The corresponding amine was obtained following the general procedure for Boc group deprotection. After evaporation, the crude residue was triturated in acetone and the resulting precipitate was filtered, washed with acetone and dried to afford the impure desired product, which was engaged in the next step without further purification.

To a stirred solution of the corresponding amine (1 equiv.) in iPrOH (0.3 M), 29 (1.5 equiv.) and Et₃N (4 equiv.) were added. The reaction mixture was stirred at 150° C. for 8 h in the microwave and concentrated under reduced pressure. The reaction was diluted with water and the aqueous layer was extracted three times with DCM. The combined organic layers were washed five times with water, once with brine, dried over MgSO₄, filtered and concentrated under reduced pressure. The crude residue was purified by flash column chromatography (DCM/MeOH=100:5 to 100:8 to 100:10 to 100:13 to 100:20) to afford the desired product as a brown solid (6% yield over three steps). Mp: 61-62° C.; HRMS (ESI): m/z: calcd for [C₃₂H₄₄N₇O]⁺: 542.3607 found: 542.3602.

### N-Benzyl-6-chloropyrimidin-4-amine (29)

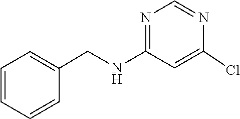 [SMILES: Clc1cc(NCc2ccccc2)ncn1]

To a stirred solution of 4,6-dichloro-pyrimidine (5 g, 33.6 mmol) in iPrOH (100 mL), benzylamine (1.2 equiv., 40.3 mmol, 4.4 mL) and Et₃N (1.2 equiv., 40.3 mmol, 5.59 mL) were added. The reaction mixture was stirred at 25° C. for 3 d and concentrated under reduced pressure. The crude residue was triturated in water, filtered and dried to afford the desired product as a beige solid (7.21 g, 98% yield). LRMS (ESI) m/z calcd for [C₁₁H₁₁ClN₃]⁺: 220.1 found: 220.1

### 9-(6-(Benzylamino)pyrimidin-4-yl)-4-(6-((4,4-dimethylpiperidin-1-yl)methyl)pyridin-3-yl)-1,4,9-triazaspiro[5.5]undecan-2-one (8)

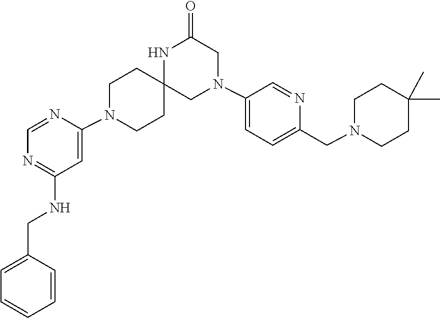 [SMILES: CC1(C)CCN(Cc2ccc(N3CC(=O)NC4(CCN(c5cc(NCc6ccccc6)ncn5)CC4)C3)cn2)CC1]

Compound 8 was obtained following the general procedure for S_{N}Ar with chloropyrimidine derivatives (chromatography: DCM/MeOH=100:5 to 100:10 to 100:15 NH to 100:20 to 100:30). The obtained impure product was triturated in diethyl ether, filtered, washed twice with ether, once with water and once with ether to afford the desired product as a white solid (14 mg, 25% yield). Mp: 208-209° C.; HRMS (ESI): m/z: calcd for [C₃₂H₄₃N₈O]⁺: 555.3560 found: 555.3554.

### 9-(6-(Benzylamino)pyrimidin-4-yl)-4-(4-((4,4-dimethylpiperidin-1-yl)methyl)phenyl)-1,4,9-triazaspiro[5.5]undecan-2-one (9)

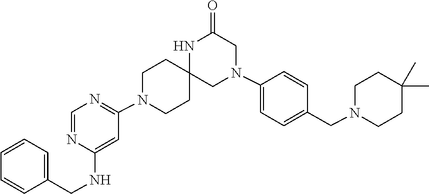 [SMILES: CC1(C)CCN(Cc2ccc(N3CC(=O)NC4(CCN(c5cc(NCc6ccccc6)ncn5)CC4)C3)cc2)CC1]

The corresponding chloropyrimidine was obtained following the general procedure for S_{N}Ar with 4,6-dichloropyrimidine. Instead of chromatography, after evaporation, the crude residue was triturated in water, filtered and washed once with water. The obtained sticky solid was dissolved in MeOH and concentrated under reduced pressure to afford the desired product, which was engaged in the next step without further purification.

Compound 9 was obtained following the general procedure for S_{N}Ar with chloropyrimidine derivatives (chromatography: DCM/MeOH=100:0 to 100:8 in 20 min, 100:8 for 10 min, 100:8 to 100:10 in 10 min). The obtained impure product was triturated in water, filtered, washed once with water to afford the desired product as a pale yellow solid (5% yield over two steps). Mp: 228-231° C.; HRMS (ESI): m/z: calcd for [C₃₃H₄₄N₇O]⁺: 554.3607 found: 554.3602.

### 4-(4-((4,4-dimethylpiperidin-1-yl)methyl)phenyl)-9-(6-(methylamino)pyrimidin-4-yl)-1,4,9-triazaspiro[5.5]undecan-2-one (10)

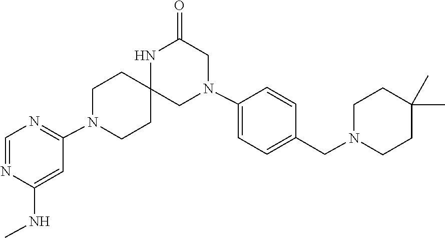

The corresponding chloropyrimidine batch was the same than the one used for compound 9.

Compound 10 was obtained following the general procedure for S_{N}Ar with chloropyrimidine derivatives (chromatography: DCM/MeOH=100:0 to 100:12 in 20 min, 100:12 for 10 min, 100:12 to 100:15 in 10 min, 100:15 for 10 min). White solid (3% yield over two steps). Mp: 235-236° C.; HRMS (ESI): m/z: calcd for [C₂₇H₄₀N₇O]⁺: 478.3294 found: 478.3289.

### 4-(4-((4,4-Dimethylpiperidin-1-yl)methyl)phenyl)-9-(7H-pyrrolo[2,3-d]pyrimidin-4-yl)-1,4,9-triazaspiro[5.5]undecan-2-one (17)

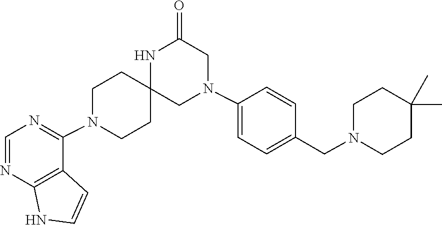 [SMILES: CC1(C)CCN(Cc2ccc(N3CC(=O)NC4(CCN(c5ncnc6[nH]ccc56)CC4)C3)cc2)CC1]

To a stirred solution of 36 (100 mg, 0.21 mmol) in dry THF (700 μL), under a nitrogen atmosphere, 4-chloro-7H-pyrrolo[2,3-d]pyrimidine (1 equiv., 0.21 mmol, 32 mg) was added. Nitrogen gas was bubbled through the reaction for two minutes and Ruphos Pd G4 (5 mol %, 0.011 mmol, 8.9 mg), Ruphos (5 mol %, 0.011 mmol, 5.1 mg) and LiHMDS (6.6 equiv., 1.39 mmol, 1.39 mL, 1 M THF) were added. The reaction mixture was stirred at 65° C. for 4 h, cooled down to 25° C. and concentrated under reduced pressure. The crude residue was purified by flash column chromatography (DCM/MeOH=100:3 to 100:5 to 100:8 to 100:10 to 100:15) to afford the desired product as a yellow solid (37 mg, 36% yield). Mp: 250-252° C.; HRMS (ESI): m/z: calcd for [C₂₈H₃₈N₇O]⁺: 488.3138. found: 488.3132.

### 9-(2-Chloro-7H-pyrrolo[2,3-d]pyrimidin-4-yl)-4-(4-((4,4-dimethylpiperidin-1-yl)methyl)phenyl)-1,4,9-triazaspiro[5.5]undecan-2-one (19)

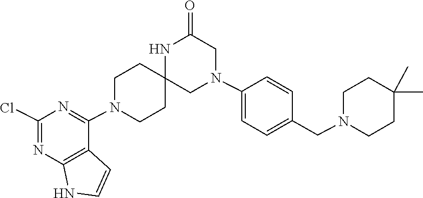 [SMILES: CC1(C)CCN(Cc2ccc(N3CC(=O)NC4(CCN(c5nc(Cl)nc6[nH]ccc56)CC4)C3)cc2)CC1]

To a stirred solution of 36 (100 mg, 0.21 mmol) in iPrOH (1 mL), 2,4-dichloro-7H-pyrrolo[2,3-d]pyrimidine (1.2 equiv., 0.25 mmol, 47 mg) and Et₃N (4 equiv., 0.84 mmol, 116 μL) were added. The reaction mixture was stirred at 100° C. for 3 h and an additional 3 h at 130° C., both in the microwave. The reaction mixture was concentrated under reduced pressure and the crude residue was triturated in water. The resulting precipitate was filtered, washed with water and dried to afford the desired impure product, which was further purified by flash column chromatography (DCM/MeOH=100:0 to 100:10 in 15 min, 100:10 for 10 min) to afford the desired product as a beige solid (46 mg, 42% yield). Mp: 199-201° C.; HRMS (ESI): m/z: calcd for [C₂₈H₃₇ClN₇O]⁺: 522.2748 found: 522.2731.

### 9-(6-(Cyclopropylamino)pyrimidin-4-yl)-4-(4-((4,4-dimethylpiperidin-1-yl)methyl)phenyl)-1,4,9-triazaspiro[5.5]undecan-2-one (16)

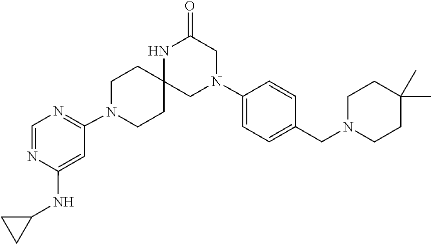 [SMILES: CC1(C)CCN(Cc2ccc(N3CC(=O)NC4(CCN(c5cc(NC6CC6)ncn5)CC4)C3)cc2)CC1]

Compound 16 was obtained following the general procedure for S_{N}Ar with chloropyrimidine derivatives (chromatography: DCM/MeOH=100:0 to 100:10 in 15 min, 100:10 for 10 min, 100:10 to 100:15 in 15 min). Brown solid (25% yield). Mp: 172-174° C.; HRMS (ESI): m/z: calcd for [C₂₉H₄₂N₇O]⁺: 504.3451 found: 504.3445.

### 4-(4-((4,4-Dimethylpiperidin-1-yl)methyl)phenyl)-9-(6-(isopropylamino)pyrimidin-4-yl)-1,4,9-triazaspiro[5.5]undecan-2-one (15)

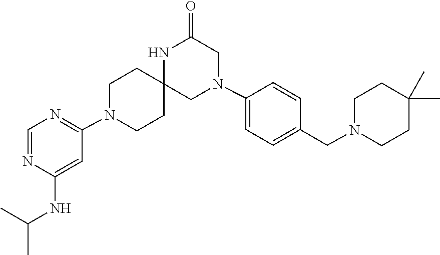 [SMILES: CC(C)Nc1cc(N2CCC3(CC2)CN(c2ccc(CN4CCC(C)(C)CC4)cc2)CC(=O)N3)ncn1]

Compound 15 was obtained following the general procedure for S_{N}Ar with chloropyrimidine derivatives (chromatography: DCM/MeOH=100:0 to 100:8 in 15 min, 100:8 for 10 min, 100:8 to 100:10 in 10 min). Beige solid (52% yield). Mp: 175-176° C.; HRMS (ESI): m/z: calcd for [C29H₄₄N₇O]⁺: 506.3607 found: 506.3602.

### 1-(4-Bromobenzyl)-4,4-dimethylpiperidine (23)

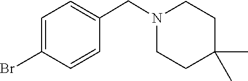 [SMILES: CC1(C)CCN(Cc2ccc(Br)cc2)CC1]

Intermediate 23 was obtained following the general procedure for dimethylpiperidine alkylation (chromatography: EtOAc/heptane=0:10 to 3:7). Yellow oil, 99% yield. LRMS (ESI) m/z calcd for [C₁₄H₂₁BrN]⁺: 282.1 found: 282.1.

### 5-Chloro-2-((4,4-dimethylpiperidin-1-yl)methyl)pyridine (26)

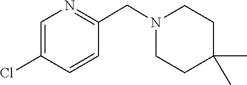 [SMILES: CC1(C)CCN(Cc2ccc(Cl)cn2)CC1]

To a stirred solution of (5-chloropyridin-2-yl)methanol (2.43 g, 17 mmol) in DCM (40 mL), SOCl₂ (1.5 equiv., 25.5 mmol, 1.85 mL) and DMF (1 drop) were added. The reaction mixture was stirred at 25° C. for 2 h and concentrated under reduced pressure to afford the chloroalkane, which was engaged in the next step without further purification.

Intermediate 26 was obtained following the general procedure for dimethylpiperidine alkylation but the reaction mixture was stirred at 70° C. for 3 h (column chromatography: EtOAc/heptane=1:9 to 3:7). Yellow solid, 86% yield over two steps. LRMS (ESI) m/z calcd for [C₁₃H₂₀ClN₂]⁺: 239.1 found: 239.2

### 9-(6-Chloropyrimidin-4-yl)-4-(6-((4,4-dimethylpiperidin-1-yl)methyl)pyridin-3-yl)-1,4,9-triazaspiro[5.5]undecan-2-one (34)

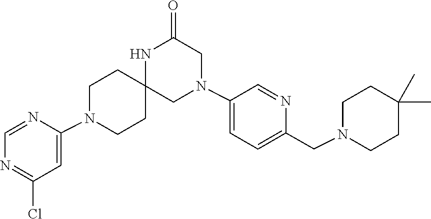 [SMILES: CC1(C)CCN(Cc2ccc(N3CC(=O)NC4(CCN(c5cc(Cl)ncn5)CC4)C3)cn2)CC1]

The corresponding Boc protected amine was obtained following the general procedure for Buchwald-Hartwig coupling (chromatography: DCM/MeOH=100:2 to 100:5 to 100:8 to 100:12 to 100:15 to 100:20). The impure desired product was engaged in the next step without further purification.

The corresponding amine was obtained following the general procedure for Boc group deprotection. After evaporation, the crude residue was triturated in acetone and the resulting precipitate was filtered, washed with acetone and dried to afford the impure desired product, which was engaged in the next step without further purification.

Intermediate 34 was obtained following the general procedure for S_{N}Ar with 4,6-dichloropyrimidine (column chromatography: DCM/MeOH=100:2 to 100:5 to 100.8 to 100:10 to 100:15 to 100:20) to afford the desired product as a yellow solid (123 mg, 27% yield over three steps). LRMS (ESI) m/z calcd for [C₂₅H₃₅ClN₇O]⁺: 484.3 found: 484.3.

### tert-Butyl 4-(4-((4,4-dimethylpiperidin-1-yl)methyl)phenyl)-2-oxo-1,4,9-triazaspiro[5.5]undecane-9-carboxylate (35)

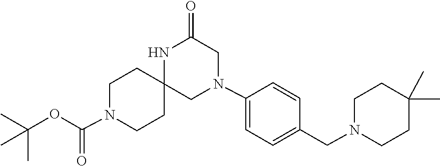 [SMILES: CC1(C)CCN(Cc2ccc(N3CC(=O)NC4(CCN(C(=O)OC(C)(C)C)CC4)C3)cc2)CC1]

Intermediate 35 was obtained following the general procedure for Buchwald-Hartwig coupling (chromatography: DCM/MeOH=100:5 to 100:8 to 100:12 to 100:20). Beige solid, 93% yield.

### 4-(4-((4,4-Dimethylpiperidin-1-yl)methyl)phenyl)-1,4,9-triazaspiro[5.5]undecan-2-one hydrochloride (36)

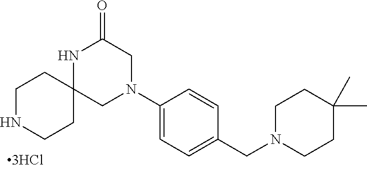 [SMILES: CC1(C)CCN(Cc2ccc(N3CC(=O)NC4(CCNCC4)C3)cc2)CC1]

Intermediate 36 was obtained following the general procedure for Boc group deprotection. After evaporation, the crude residue was triturated in acetone and the resulting precipitate was filtered, washed with acetone and dried to afford the desired product as a beige solid (50% yield over two steps from intermediate 32). LRMS (ESI) m/z calcd for [C₂₂H₃₅N₄O]⁺: 371.3 found: 371.3.

### 9-(6-Chloropyrimidin-4-yl)-4-(4-((4,4-dimethylpiperidin-1-yl)methyl)phenyl)-1,4,9-triazaspiro[5.5]undecan-2-one (37)

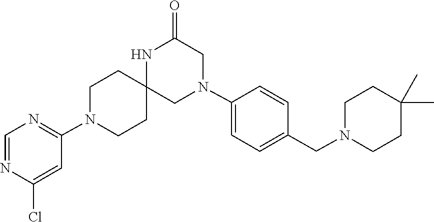 [SMILES: CC1(C)CCN(Cc2ccc(N3CC(=O)NC4(CCN(c5cc(Cl)ncn5)CC4)C3)cc2)CC1]

Intermediate 37 was obtained following the general procedure for S_{N}Ar with 4,6-dichloropyrimidine. Instead of chromatography, after evaporation of the crude mixture, the residue was triturated in water. The obtained precipitate was filtered, washed with water and dried to afford the desired product as a brown solid (63% yield). LRMS (ESI) m/z calcd for [C₂₆H₃₆ClN₆O]⁺: 483.3 found: 483.3.

### 4-(4-((4,4-Dimethylpiperidin-1-yl)methyl)-2-fluorophenyl)-9-(6-(methylamino)pyrimidin-4-yl)-1,4,9-triazaspiro[5.5]undecan-2-one (20)

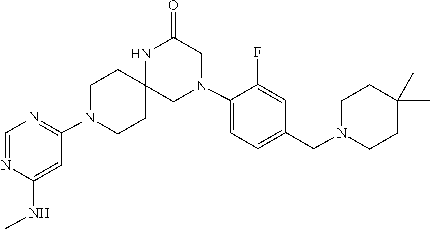

Compound 20 was obtained following the general procedure for S_{N}Ar with chloropyrimidine derivatives (chromatography: DCM/MeOH=100:0 to 100:8 in 20 min, 100:8 for 15 min). White solid (47% yield). Mp: 202-204° C.; HRMS (ESI): m/z: calc or [C₂₇H₃₉FN₇O]⁺: 496.3200 found: 496.3195.

### 4-(4-((4,4-Dimethylpiperidin-1-yl)methyl)-3-fluorophenyl)-9-(6-(methylamino)pyrimidin-4-yl)-1,4,9-triazaspiro[5.5]undecan-2-one (21)

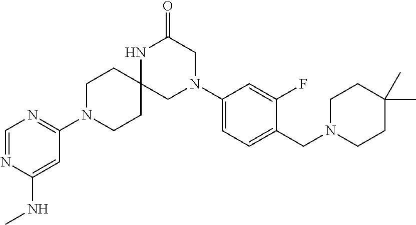

Compound 21 was obtained following the general procedure for S_{N}Ar with chloropyrimidine derivatives (chromatography: DCM/MeOH=100:0 to 100:8 in 20 min, 100:8 for 5 min, 100:8 to 100:12 in 10 min, 100:12 for 5 min). Yellow solid, 69% yield. Mp: 210-211° C.; HRMS (ESI): m/z: calcd for [C₂₇H₃₉FN₇O]⁺: 496.3200 found: 496.3195.

### 4-(4-((4,4-Dimethylpiperidin-1-yl)methyl)-2,5-difluorophenyl)-9-(6-(methylamino)pyrimidin-4-yl)-1,4,9-triazaspiro[5.5]undecan-2-one (22)

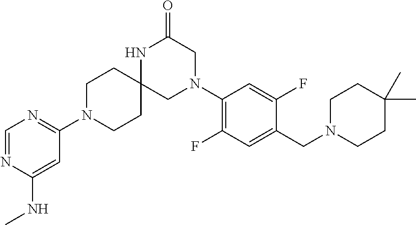

The corresponding Boc protected amine was obtained following the general procedure for Buchwald-Hartwig coupling (chromatography: DCM/MeOH=100:3 to 100:5 to 100:8). The impure desired product was engaged in the next step without further purification.

The corresponding amine was obtained following the general procedure for Boc group deprotection. The impure desired product was engaged in the next step without further purification.

The corresponding chloropyrimidine was obtained following the general procedure for S_{N}Ar with 4,6-dichloropyrimidine. The impure desired product was engaged in the next step without further purification.

UZH2 was obtained following the general procedure for S_{N}Ar with chloropyrimidine derivatives (chromatography: DCM/MeOH=100:0 to 100:10 in 15 min, to 100:10 for 10 min, 100:10 to 100:12 in 10 min. White solid, 56% yield over four steps. Mp: 214-216; HRMS (ESI): m/z: calcd for [C27H₃₈F₂N₇O]⁺: 514.3106 found: 514.3100.

### 1-(4-Bromo-3-fluorobenzyl)-4,4-dimethylpiperidine (47)

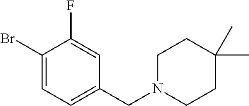 [SMILES: CC1(C)CCN(Cc2ccc(Br)c(F)c2)CC1]

Intermediate 47 was obtained following the general procedure for dimethylpiperidine alkylation (column chromatography: EtOAc/heptane=1:9). Colorless oil, 98% yield. LRMS (ESI) m/z calcd for [C₁₄H₂₀BrFN]⁺: 300.1 found: 300.1.

### 9-(6-Chloropyrimidin-4-yl)-4-(4-((4,4-dimethylpiperidin-1-yl)methyl)-2-fluorophenyl)-1,4,9-triazaspiro[5.5]undecan-2-one (48)

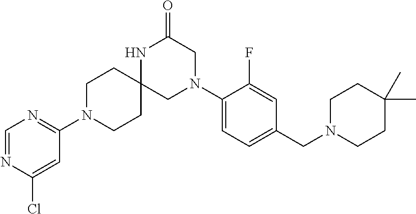 [SMILES: CC1(C)CCN(Cc2ccc(N3CC(=O)NC4(CCN(c5cc(Cl)ncn5)CC4)C3)c(F)c2)CC1]

The corresponding Boc protected amine was obtained following the general procedure for Buchwald-Hartwig coupling (chromatography: DCM/MeOH=100:3 to 100:5 to 100:8). The impure desired product was engaged in the next step without further purification.

The corresponding amine was obtained following the general procedure for Boc group deprotection. The impure desired product was engaged in the next step without further purification.

Intermediate 48 was obtained following the general procedure for S_{N}Ar with 4,6-dichloropyrimidine (column chromatography: DCM/MeOH=100:3 to 100:5 to 100.8 to 100:10) to afford the desired product as a white solid (15% yield over three steps). LRMS (ESI) m/z calcd for [C₂₆H₃₅ClFN₆O]: 501.3 found: 501.3.

### 1-(4-Bromo-2-fluorobenzyl)-4,4-dimethylpiperidine (49)

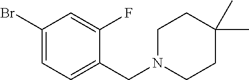 [SMILES: CC1(C)CCN(Cc2ccc(Br)cc2F)CC1]

Intermediate 49 was obtained following the general procedure for dimethylpiperidine alkylation. Colorless liquid, 99% yield. LRMS (ESI) m/z calcd for [C₁₄H₂₀BrFN]⁺: 300.1 found: 300.1.

### tert-Butyl 4-(4-((4,4-dimethylpiperidin-1-yl)methyl)-3-fluorophenyl)-2-oxo-1,4,9-triazaspiro[5.5]undecane-9-carboxylate (50)

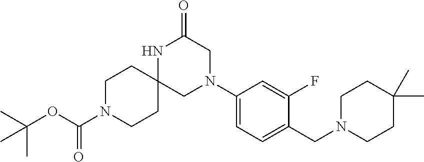 [SMILES: CC1(C)CCN(Cc2ccc(N3CC(=O)NC4(CCN(C(=O)OC(C)(C)C)CC4)C3)cc2F)CC1]

Intermediate 50 was obtained following the general procedure for Buchwald-Hartwig coupling (chromatography: DCM/MeOH=100:5 to 100:8 to 100:12 to 100:16). Brown solid (83% yield). LRMS (ESI) m/z calcd for [C₂₇H₄₂FN₄O₃]⁺: 489.3 found: 489.4.

### 9-(6-Chloropyrimidin-4-yl)-4-(4-((4,4-dimethylpiperidin-1-yl)methyl)-3-fluorophenyl)-1,4,9-triazaspiro[5.5]undecan-2-one (51)

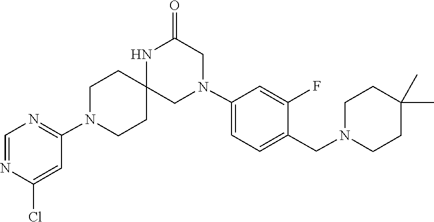 [SMILES: CC1(C)CCN(Cc2ccc(N3CC(=O)NC4(CCN(c5cc(Cl)ncn5)CC4)C3)cc2F)CC1]

The corresponding amine was obtained following the general procedure for Boc group deprotection. The impure desired product was engaged in the next step without further purification.

Intermediate 51 was obtained following the general procedure for S_{N}Ar with 4,6-dichloropyrimidine. Instead of chromatography, after evaporation of the crude mixture, the residue was triturated in water. The obtained precipitate was filtered, washed with water and dried to afford the desired product as a brown solid (58% yield over two steps). LRMS (ESI) m/z calcd for [C₂₆H₃₅ClFN₆O]: 501.3 found: 501.3.

### 1-(4-Bromo-2,5-difluorobenzyl)-4,4-dimethylpiperidine (52)

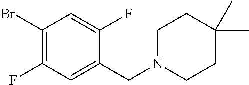 [SMILES: CC1(C)CCN(Cc2cc(F)c(Br)cc2F)CC1]

To a stirred solution of 4-bromo-2,5-difluorobenzoic acid (1 g, 4.2 mmol) in dry THF (10 mL), under a nitrogen atmosphere, BH₃·SMe₂ (2 equiv., 8.4 mmol, 4.2 mL, 2 M THF) was added. The reaction mixture was stirred for 17 h at 25° C., cooled down to 0° C. and quenched by the addition of a saturated aqueous Na₂CO₃ solution. The aqueous layer was extracted three times with EtOAc and the combined organic layers were washed once with brine, dried over MgSO₄ and concentrated under reduced pressure to afford the desired product as a brown solid (789 mg, 83% yield).

To a stirred solution of the corresponding alcohol (789 mg, 3.54 mmol) in DCM (10 mL), SOCl₂ (1.5 equiv., 5.3 mmol, 385 μL) and DMF (1 drop) were added. The reaction mixture was stirred at 25° C. for 3 h and concentrated under reduced pressure to afford the desired chloroalkane, which was engaged in the next step without further purification.

To a stirred solution of the corresponding chloroalkane (425 mg, 1.76 mmol) in dimethylformamide (5 mL), 4,4-dimethylpiperidine hydrochloride (1 equiv., 1.76 mmol, 263 mg) and K₂CO₃ (2 equiv. 3.52 mmol, 486 mg) were added. The reaction mixture was stirred at 25° C. for 3 days and concentrated under reduced pressure. The obtained residue was purified by flash column chromatography (EtOAc/heptane=3:100 to 10:100) to afford the desired product as a colorless liquid (514 mg, 92%). LRMS (ESI) m/z calcd for [C₁₄H₁₉BrF₂N]⁺: 318.1 found: 318.1

### 8-(4-((4,4-Dimethylpiperidin-1-yl)methyl)phenyl)-2-(6-(methylamino)pyrimidin-4-yl)-2,5,8-triazaspiro[3.5]nonan-6-one (11)

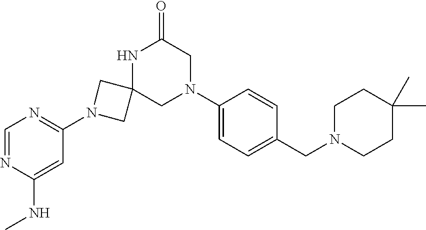 [SMILES: CNc1cc(N2CC3(CN(c4ccc(CN5CCC(C)(C)CC5)cc4)CC(=O)N3)C2)ncn1]

The corresponding Boc protected amine was obtained following the general procedure for Buchwald-Hartwig coupling (chromatography: DCM/MeOH=100:5 to 100:8 to 100:11 to 100:15). The impure desired product was engaged in the next step without further purification.

The corresponding amine was obtained following the general procedure for Boc group deprotection. The impure desired product was engaged in the next step without further purification.

The corresponding chloropyrimidine was obtained following the general procedure for S_{N}Ar with 4,6-dichloropyrimidine. Due to 40 derivative impurities still present, 7 equivalents of the pyrimidine and 7 equivalents of Et₃N were used and the reaction was heated for 7 h at 80° C. in the microwave. The impure desired product was engaged in the next step without further purification.

Compound 11 was obtained following the general procedure for S_{N}Ar with chloropyrimidine derivatives (chromatography: DCM/MeOH=100:0 to 100:10 in 15 min, 100:10 for 5 min, 100:10 to 100:13 in 5 min, 100:13 for 5 min). Yellow solid, 19% yield over four steps. Mp: Decomposition; HRMS (ESI): m/z: calcd for [C₂₅H₃₆N₇O]⁺: 450.2981 found: 450.2976.

### tert-Butyl 3-amino-3-(nitromethyl)azetidine-1-carboxylate (38)

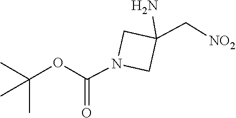 [SMILES: CC(C)(C)OC(=O)N1CC(N)(C[N+](=O)[O-])C1]

To a stirred solution of tert-butyl 3-oxoazetidine-1-carboxylate (10.65 g, 62 mmol) in EtOH (31 mL), MeNO₂ (13 mL) and K₂CO₃ (1 mol %, 0.62 mmol, 86 mg) were added. The reaction mixture was stirred at 25° C. for 17 h and filtered. The filtrate was concentrated under reduced pressure to afford the desired product, which was engaged in the next step without further purification.

To a stirred solution of the corresponding alcohol (62 mmol) in dry DCM (250 mL), under a nitrogen atmosphere and cooled to −78° C., DAST (1.2 eq., 74.4 mmol, 9.8 mL) was added dropwise. The cooling bath was removed and the reaction mixture was stirred for 3 h, cooled to 0° C. and quenched slowly by the addition of a saturated aqueous NaHCO₃ solution. The aqueous layer was extracted three times with DCM, washed once with brine, dried over MgSO₄, filtered and concentrated under reduced pressure to obtain the desired product, which was engaged in the next step without further purification.

The corresponding nitromethylene (62 mmol) was dissolved in ammonia (17.7 mL, 7 N in MeOH) and the reaction mixture was stirred for 2 h at 25° C. The reaction mixture was concentrated under reduced pressure to afford the desired product as an orange solid (15.67 g, quantitative yield over three steps).

### tert-Butyl 3-(aminomethyl)-3-(((benzyloxy)carbonyl)amino)azetidine-1-carboxylate (39)

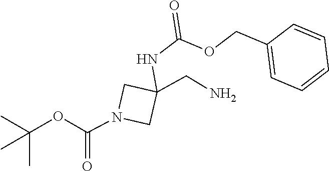 [SMILES: CC(C)(C)OC(=O)N1CC(CN)(NC(=O)OCc2ccccc2)C1]

To a stirred solution of 38 (62 mmol) in dichloromethane (100 mL), a solution of NaHCO₃ (2 equiv., 124 mmol, 10.42 g) in water (100 mL) was added. The reaction mixture was cooled to 0° C. and CbzCl (1 equiv., 62 mmol, 8.8 mL) was added dropwise. The reaction mixture was stirred at 25° C. for 17 h and the two phases were separated. The aqueous layer was extracted two times with DCM. The combined organic layers were washed once with brine, dried over MgSO₄, filtered and concentrated reduced pressure to afford the desired product, which was engaged in the next step without further purification.

To a stirred solution of the corresponding nitroalkane (62 mmol) in dry MeOH (300 mL), under a nitrogen atmosphere at 0° C., NiCl₂·6H₂O (1 equiv., 62 mmol, 16.9 g) was added, followed by NaBH₄ (5 equiv., 310 mmol, 11.7 g) portionwise to avoid strong H₂ evolution. Caution when adding NaBH₄, the reaction is highly exothermic and produce hydrogen gas. The reaction mixture was stirred at 25° C. for 1 h and quenched by adding saturated aqueous NaHCO₃ solution. The mixture was filtered through a pad of Celite, the filtrate was concentrated under reduced pressure and the obtained residue was diluted with brine and a saturated aqueous Na₂CO₃ solution. The aqueous layer was extracted three times with DCM and the combined organic layers were washed once with brine, dried over MgSO₄, filtered and concentrated under reduced pressure. The crude residue was purified by flash column chromatography (DCM/MeOH/NH₄OH=100:3:0 to 100:3:1 to 100:5:1 to 100:8:1 to 100:12:1 to 100:20:1) to afford the desired product as a white solid (11.3 g, 54% yield over two steps). LRMS (ESI) m/z calcd for [C₃₄H₅₁N₆O]⁺=[2M+H]⁺: 671.4 found: 671.4.

### tert-Butyl 6-oxo-2,5,8-triazaspiro[3.5]nonane-2-carboxylate (40)

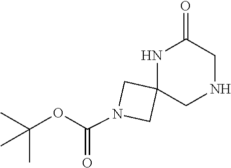 [SMILES: CC(C)(C)OC(=O)N1CC2(CNCC(=O)N2)C1]

To a stirred solution of 39 (11.3 g, 33.7 mmol) in DCM (110 mL) at 0° C., Et₃N (1 equiv., 33.7 mmol, 4.7 mL) and ethyl 2-bromoacetate (1 equiv., 33.7 mmol, 3.7 mL) were added. The reaction mixture was stirred at 25° C. for 17 h and diluted with a saturated aqueous NaHCO₃ solution. The aqueous layer was extracted three times with DCM and the combined organic layers were washed once with water, once with brine, dried over MgSO₄, filtered and concentrated under reduced pressure, to afford the impure desired product (12.4 g, 29 mmol), which was engaged in the next step without further purification.

To a stirred solution of the corresponding Cbz protected amine (29 mmol) in iPrOH (240 mL), Pd/C (5 mol %, 1.5 mmol, 1.6 g, 10% wt) and ammonium formate (6 equiv., 174 mmol, 11 g) were added portionwise. The reaction mixture was stirred at 80° C. for 4 h, cooled to 25° C., filtered through a pad of Celite and concentrated under reduced pressure. The obtained residue was partitioned between DCM and water, the two phases were separated and the aqueous layer was extracted three times with DCM. The combined organic layers were washed once with water, once with brine, dried over MgSO₄, filtered and concentrated under reduced pressure. The crude residue was purified by flash column chromatography (DCM/MeOH=100:5 to 100:8 to 100:10 to 100:15 to 100:20) to afford the desired product as a white solid (2.6 g, 32% yield over two steps). LRMS (ESI) m/z calcd for [C₇H₁₂N₃O₃]⁺=[M-tBu+2H]⁺: 186.1 found: 186.2

### 4-(4-((4-Fluoro-4-methylpiperidin-1-yl)methyl)phenyl)-9-(6-(methylamino)pyrimidin-4-yl)-1,4,9-triazaspiro[5.5]undecan-2-one (53)

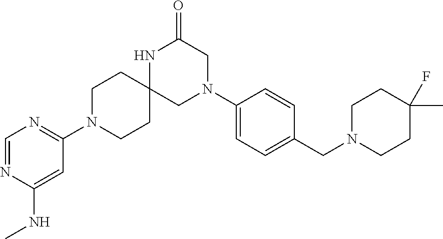 [SMILES: CNc1cc(N2CCC3(CC2)CN(c2ccc(CN4CCC(C)(F)CC4)cc2)CC(=O)N3)ncn1]

Compound 53 was obtained following the general procedure for S_{N}Ar with chloropyrimidine derivatives (chromatography: DCM/MeOH=100:0 to 100:7 in 20 min, 100:7 for 5 min, 100:7 to 100:8 in 5 min, 100:8 for 10 min). Pale yellow solid, 53% yield. Mp: 203-205° C.; HRMS (ESI): m/z: calcd for [C₂₆H₃₇FN₇O]⁺: 482.3044 found: 482.3038.

### 9-(6-Chloropyrimidin-4-yl)-4-(4-((4-fluoro-4-methylpiperidin-1-yl)methyl)phenyl)-1,4,9-triazaspiro[5.5]undecan-2-one (54)

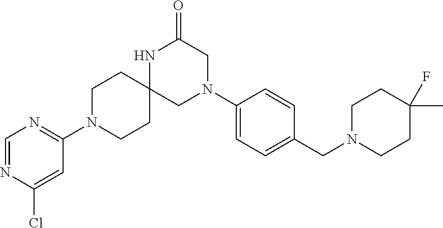 [SMILES: CC1(F)CCN(Cc2ccc(N3CC(=O)NC4(CCN(c5cc(Cl)ncn5)CC4)C3)cc2)CC1]

The corresponding Boc protected amine was obtained following the general procedure for Buchwald-Hartwig coupling (chromatography: EtOAc/heptane=7:3 to 9:1 to EtOAc/MeOH=100:3 to 100:5 to 100:8 to 100:10). The impure desired product was engaged in the next step without further purification.

The corresponding amine was obtained following the general procedure for Boc group deprotection. The impure desired product was engaged in the next step without further purification.

Intermediate 54 was obtained following the general procedure for S_{N}Ar with 4,6-dichloropyrimidine (column chromatography: DCM/MeOH=100:3 to 100:5 to 100.8) to afford the desired product as a white solid (12% yield over three steps).

### 1-(4-Bromobenzyl)-4-fluoro-4-methylpiperidine (55)

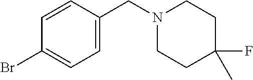 [SMILES: CC1(F)CCN(Cc2ccc(Br)cc2)CC1]

To a stirred solution of tert-butyl 4-hydroxy-4-methylpiperidine-1-carboxylate (500 mg, 2.32 mmol) in dry DCM (7 mL), at 0° C. under a nitrogen atmosphere, DAST (1.5 eq., 3.48 mmol, 460 μL) was added. The mixture was stirred at 25° C. for 3 h and quenched by adding saturated aqueous NaHCO₃ solution. The two phases were separated and the aqueous layer was extracted two times with DCM. The combined organic layers were washed with brine, dried over MgSO₄, filtered and concentrated under reduced pressure to afford the desired product, which was engaged in the next step without further purification.

The corresponding amine was obtained following the general procedure for Boc group deprotection. The impure desired product was engaged in the next step without further purification.

Intermediate 55 was obtained following the general procedure for dimethylpiperidine alkylation. (column chromatography: EtOAc/heptane=1:9 to 3:7 to 1:1). Yellow oil, 77% yield over three steps.

### 4-(4-((4,4-dimethylpiperidin-1-yl)methyl)phenyl)-9-(1H-pyrazolo[3,4-d]pyrimidin-4-yl)-1,4,9-triazaspiro[5.5]undecan-2-one (56)

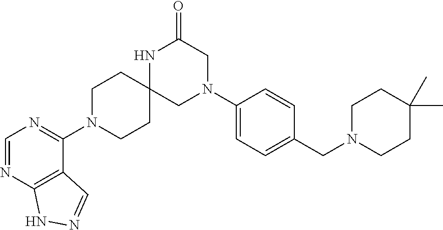 [SMILES: CC1(C)CCN(Cc2ccc(N3CC(=O)NC4(CCN(c5ncnc6[nH]ncc56)CC4)C3)cc2)CC1]

To a stirred solution of 36 (150 mg, 0.31 mmol) in iPrOH (1.5 mL), 2,4-dichloro-7H-pyrrolo[2,3-d]pyrimidine (1.2 equiv., 0.38 mmol, 58 mg) and Et₃N (4 equiv., 1.24 mmol, 172 μL) were added. The reaction mixture was stirred at 50° C. for 3 h and an additional 2 h at 70° C., both in the microwave. The reaction mixture was concentrated under reduced pressure and the crude residue was triturated in water. The resulting precipitate was filtered, washed with water, washed with DCM and dried to afford the desired impure product, which was further purified by flash column chromatography (DCM/MeOH=100:0 to 100:10 in 20 min, 100:10 for 10 min) to afford the desired product as a yellow solid (33 mg, 22% yield). Mp: 252-254° C.; HRMS (ESI): m/z: calcd for [C₂₇H₃₇N₈O]⁺: 489.3090 found: 489.3085

TABLE 8
IC50 data for N6-adenosine-methyltransferase

| | | IC₅₀ |
|---|---|---|
| Name | Structure | (μM) |
| 53 | 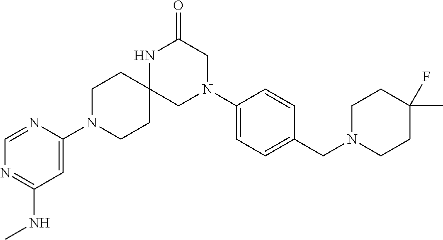 [SMILES: CNc1cc(N2CCC3(CC2)CN(c2ccc(CN4CCC(C)(F)CC4)cc2)CC(=O)N3)ncn1] | 0.069 |
| 7 | 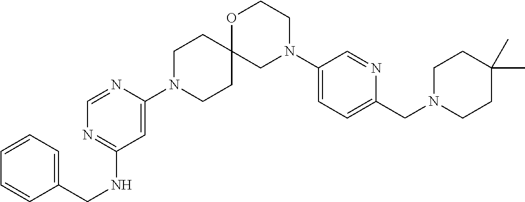 [SMILES: CC1(C)CCN(Cc2ccc(N3CCOC4(CCN(c5cc(NCc6ccccc6)ncn5)CC4)C3)cn2)CC1] | 0.28 |
| 8 | 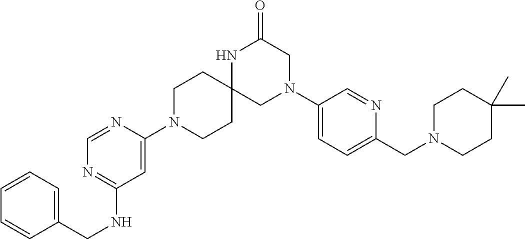 [SMILES: CC1(C)CCN(Cc2ccc(N3CC(=O)NC4(CCN(c5cc(NCc6ccccc6)ncn5)CC4)C3)cn2)CC1] | 0.037 |
| 9 | 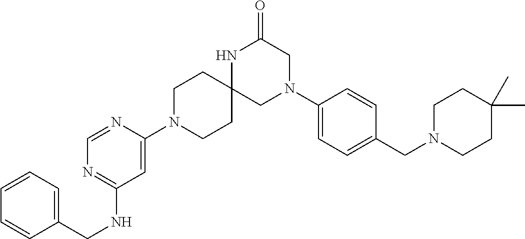 [SMILES: CC1(C)CCN(Cc2ccc(N3CC(=O)NC4(CCN(c5cc(NCc6ccccc6)ncn5)CC4)C3)cc2)CC1] | 0.026 |
| 10 | 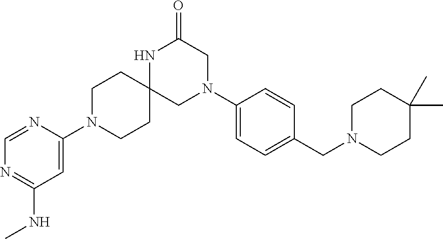 [SMILES: CNc1cc(N2CCC3(CC2)CN(c2ccc(CN4CCC(C)(C)CC4)cc2)CC(=O)N3)ncn1] | 0.089 |
| 11 | 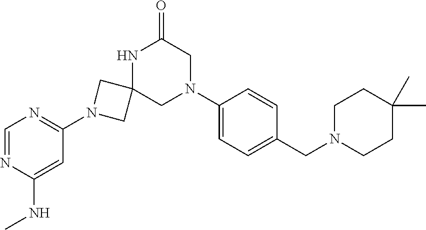 [SMILES: CNc1cc(N2CC3(CN(c4ccc(CN5CCC(C)(C)CC5)cc4)CC(=O)N3)C2)ncn1] | 0.44 |
| 17 | 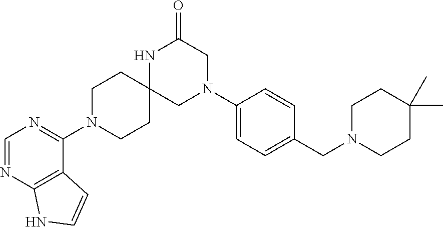 [SMILES: CC1(C)CCN(Cc2ccc(N3CC(=O)NC4(CCN(c5ncnc6[nH]ccc56)CC4)C3)cc2)CC1] | 0.061 |
| 56 | 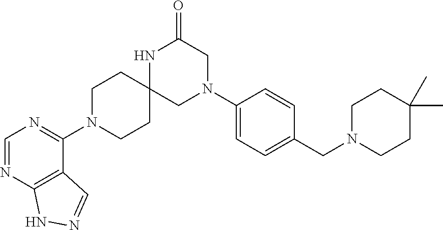 [SMILES: CC1(C)CCN(Cc2ccc(N3CC(=O)NC4(CCN(c5ncnc6[nH]ncc56)CC4)C3)cc2)CC1] | 0.14 |
| 19 | 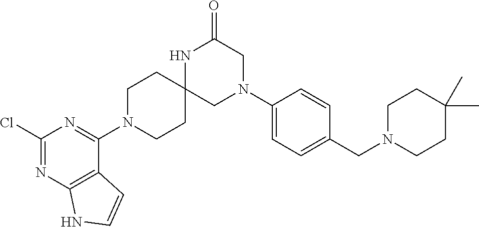 [SMILES: CC1(C)CCN(Cc2ccc(N3CC(=O)NC4(CCN(c5nc(Cl)nc6[nH]ccc56)CC4)C3)cc2)CC1] | 0.024 |
| 16 | 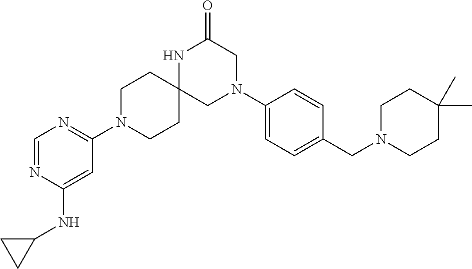 [SMILES: CC1(C)CCN(Cc2ccc(N3CC(=O)NC4(CCN(c5cc(NC6CC6)ncn5)CC4)C3)cc2)CC1] | 0.084 |
| 15 | 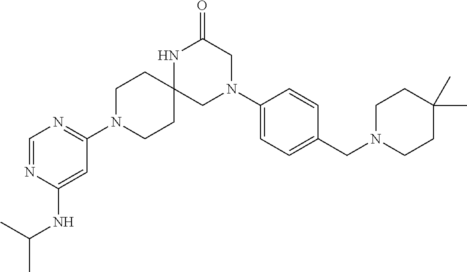 [SMILES: CC(C)Nc1cc(N2CCC3(CC2)CN(c2ccc(CN4CCC(C)(C)CC4)cc2)CC(=O)N3)ncn1] | 0.33 |
| 20 | 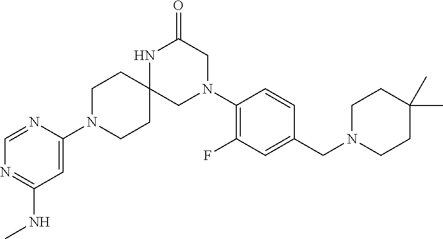 [SMILES: CNc1cc(N2CCC3(CC2)CN(c2ccc(CN4CCC(C)(C)CC4)cc2F)CC(=O)N3)ncn1] | 0.038 |
| 21 | 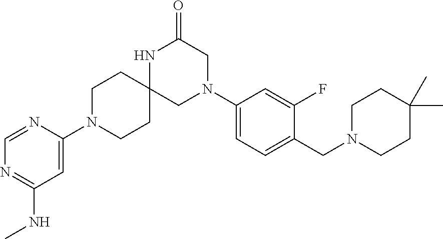 [SMILES: CNc1cc(N2CCC3(CC2)CN(c2ccc(CN4CCC(C)(C)CC4)c(F)c2)CC(=O)N3)ncn1] | 0.032 |
| 22 | 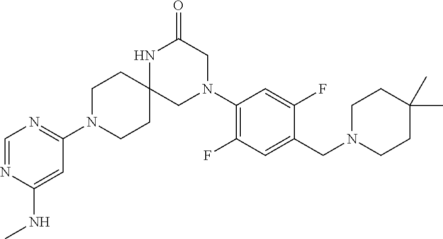 [SMILES: CNc1cc(N2CCC3(CC2)CN(c2cc(F)c(CN4CCC(C)(C)CC4)cc2F)CC(=O)N3)ncn1] | 0.008 |

TABLE 9
Exemplary PROTAC compounds IC50 data for N6-adenosine-methyltransferase

| | | IC₅₀ |
|---|---|---|
| Name | Structure | (μM) |
| 53-1 | 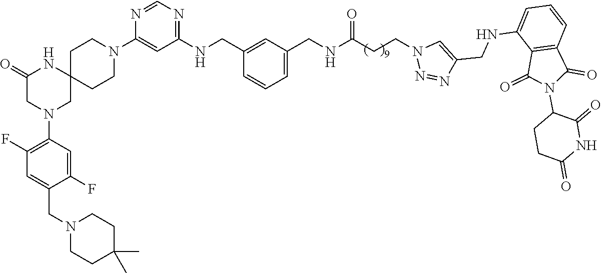 | 0.20 |
| | 53-1 | |
| 52-1 | 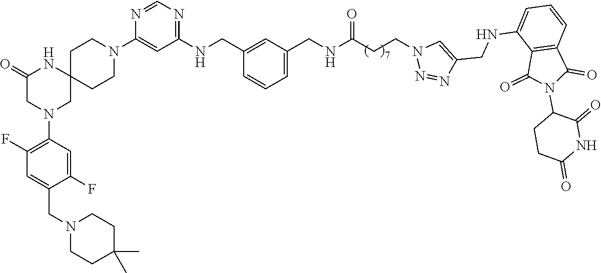 | 0.088 |
| | 52-1 | |
| 59-1 | 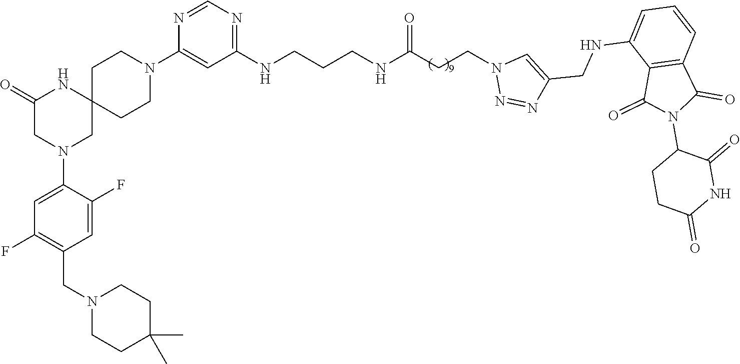 | 0.22 |
| | 59-1 | |
| 58-1 | 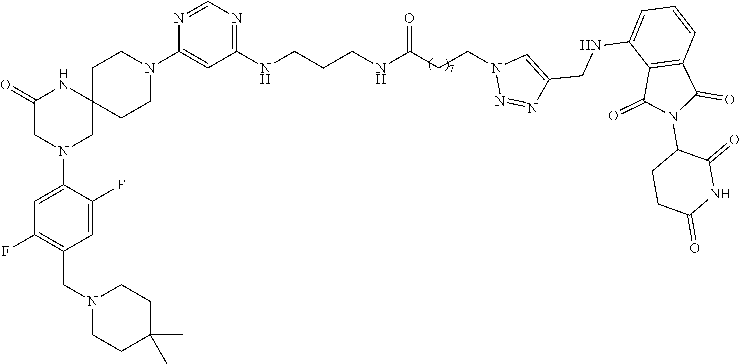 | 0.067 |
| | 58-1 | |
| 50-1 | 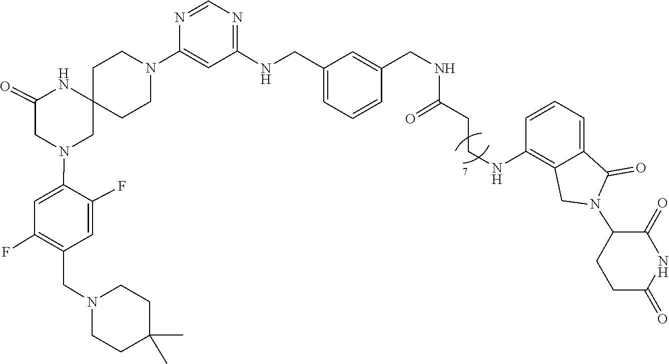 | 0.067 |
| | 50-1 | |
| 56-1 | 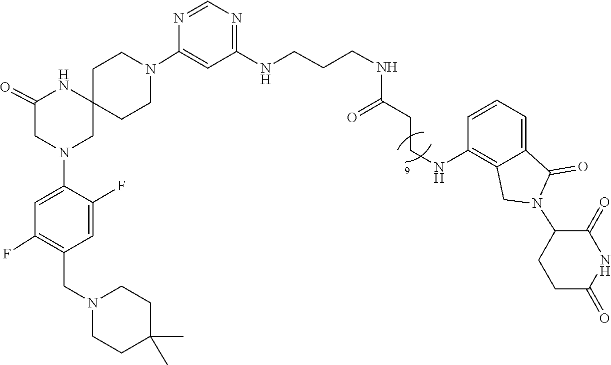 | 0.30 |
| | 56-1 | |
| 55-1 | 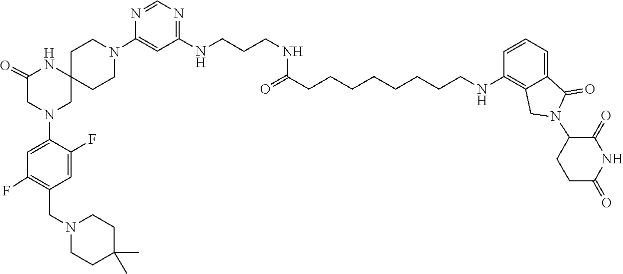 [SMILES: CC1(C)CCN(Cc2cc(F)c(N3CC(=O)NC4(CCN(c5cc(NCCCNC(=O)CCCCCCCCNc6cccc7c6CN(C6CCC(=O)NC6=O)C7=O)ncn5)CC4)C3)cc2F)CC1] | 0.066 |
| | 55-1 | |
| 8-4 | 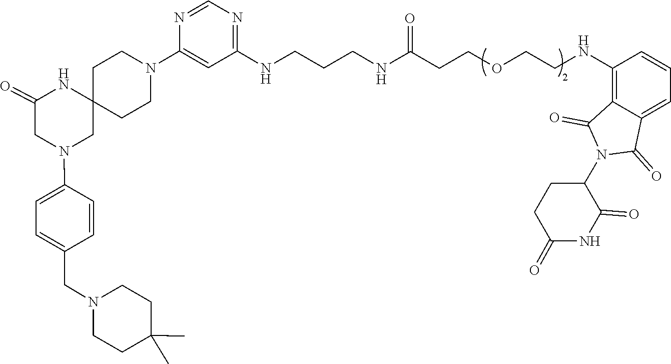 | 2.47 |
| | 8-4 | |
| 13-11 | 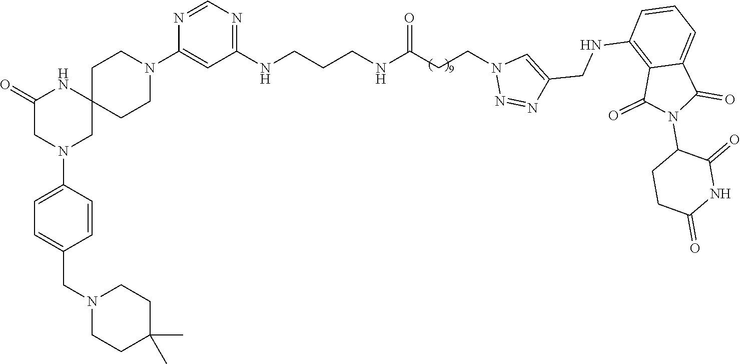 | 1.8 |
| | 13-11 | |
| 54-1 | 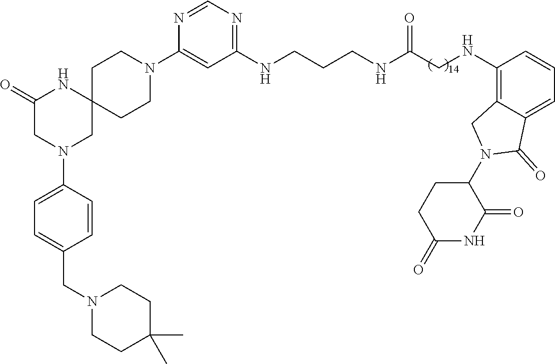 | 1.22 |
| | 54-1 | |
| 9-5 | 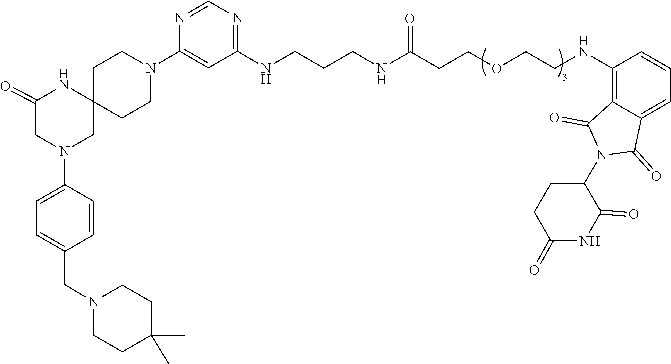 | 1.06 |
| | 9-5 | |
| 11-7 | 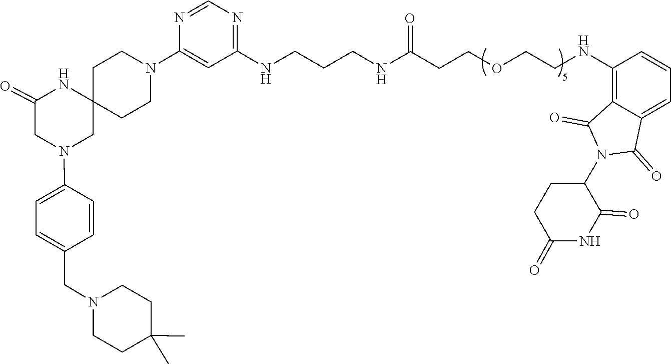 | 0.64 |
| | 11-7 | |
| 10-6 | 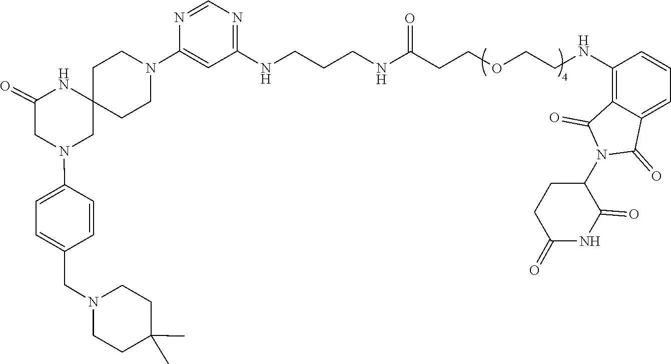 | 0.59 |
| | 10-6 | |
| 14-12 | 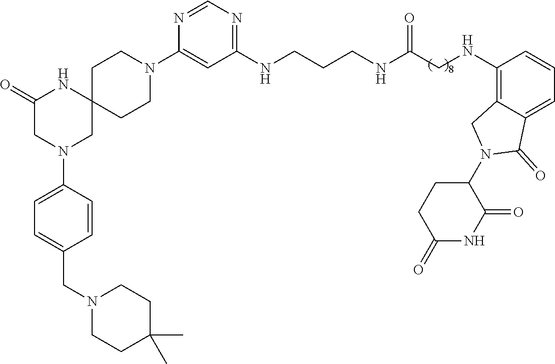 | 0.26 |
| | 14-12 | |
| 57-1 | 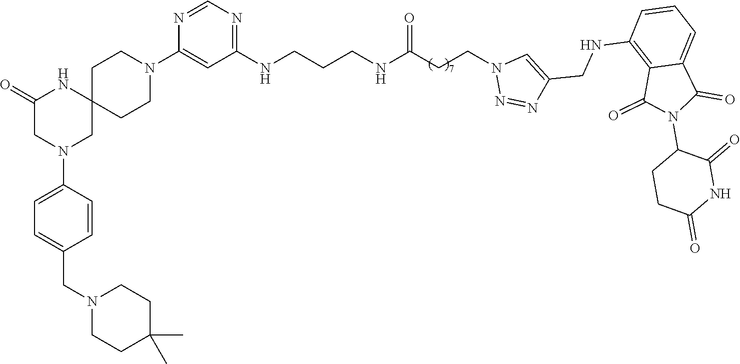 | 0.48 |
| | 57-1 | |
| 15-13 | 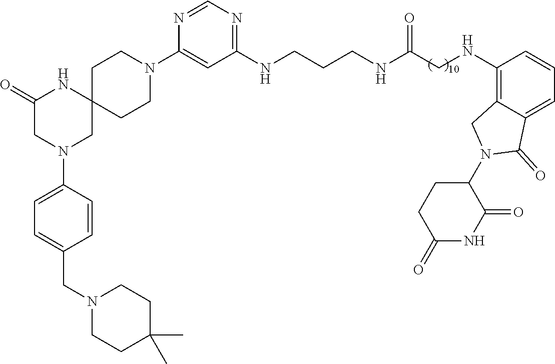 | 0.52 |
| | 15-13 | |
| 49-1 | 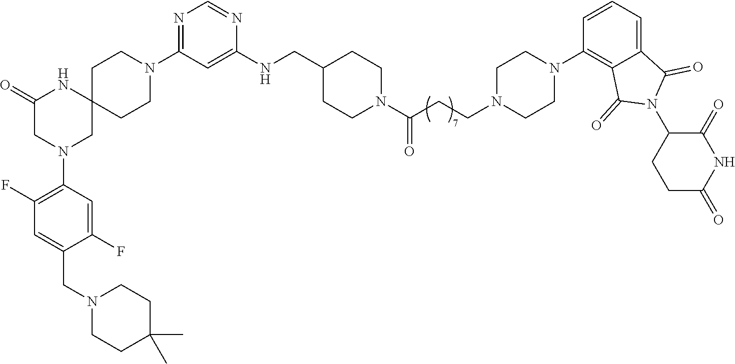 | 0.005 |
| | 49-1 | |
| 51-1 | 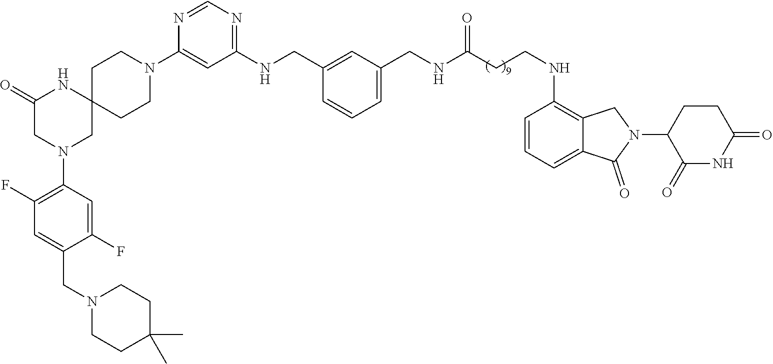 | 0.16 |
| | 51-1 | |

### Reader-Based HTRF Assay of METTL3 Inhibition In Vitro

The level of m⁶A in the oligoribonucleotide substrate after the reaction catalyzed by METTL3-MIETTL14 was quantified by measuring specific binding of the modified oligoribonucleotide to the m⁶A reader YTHDC1 by homogeneous time-resolved fluorescence (HTRF). Tested compounds that inhibit METTL3 decrease the m⁶A level and thus reduce the HTRF signal. The two-step protocol of the METTL3-METTL14 assay consists of a reaction step and subsequent detection step.

In the reaction step, METTL3-METTL14 (3 nM final concentration) methylates the 5′-biotinylated ssRNA (5′-AAGAACCGGACUAAGCU-3′ (Microsynth)) (50 nM final concentration). The co-substrate SAM (Cisbio, 62SAHZLD) was added as the last component and thus initiated the methylation reaction. The final reaction volume was 15 μL in 20 mM Tris-HCl, pH 7.5, 0.01% (w/v) bovine serum albumin (BSA). The reaction was let to incubate for 40 min at room temperature (RT) and then stopped by addition of 5 μL detection buffer (50 mM HEPES, pH 7.5, 150 mM NaCl, 200 mM KF, 0.05% (w/v) BSA, 25 nM GST-tagged m⁶A reader YTHDC1(345-509), 3 nM XL665-conjugated streptavidin (Cisbio, 610SAXLB), 1× anti-GST Eu³⁺-labeled antibody (from 400× stock (Cisbio, 61GSTKLB))). Capture of the m⁶A-modified RNA by the m⁶A reader and the biotinylated RNA by Streptavidin was allowed to proceed for 3 h at RT and in the dark before the TR-FRET signal was measured using a Tecan Spark plate reader (Tecan). The plate reader recorded with a delay of 100 s the emission at 620 and 665 nm after the excitation of the HTRF donor with UV light at 320 nm. This time-resolved measurement eliminates the short-lived background fluorescence of the signal. The emission signal was read over an integration time of 400 s. The ratio of 665/620 nm signals (HTRF ratio) allows correction for well-to-well variations and media absorbency by colored compounds. Response curves were plotted in GraphPad Prism 8.4 and fitted with nonlinear regression “log(inhibitor) vs. normalized response-variable slope”, from which IC50 values were determined. Each compound was measured in triplicates.

The results of the IC50 values are shown in Table 9.

Screening PROTACs Activity with Western Blot

METTL3 (and METTL14) protein degradation was monitored using Western blot. Cells were treated with the indicated concentration of PROTACs (or DMSO, control) for 24 h, 37° C. with 5% CO₂. Samples were then collected and lysed with RIPA buffer with protease inhibitors (11697498001, Roche). After SDS-PAGE, proteins were transferred to a nitrocellulose membrane, blocked (with 5% milk, 0.5% BSA in TBST buffer) and incubated overnight with primary antibodies. The following antibodies were used: GAPDH (#2118, Cell Signaling, 1:4000), β-actin (ab8226, Abcam, 1:2000), METTL3 (ab195352, Abcam, 1:1000), METTL14 (ab220031, Abcam, 1:1000). Membranes were scanned using LI-COR Odyssey DLx Imager after incubation with appropriate secondary antibodies (anti-mouse IgG IRDye® 680RD (926-68072, LI-COR, 1:10000), Goat anti-Rabbit IgG IRDye® 800CW (926-32211, LI-COR, 1:10000)). Densitometry was performed in Image Studio Lite software and analysis in GraphPad Prism 9.

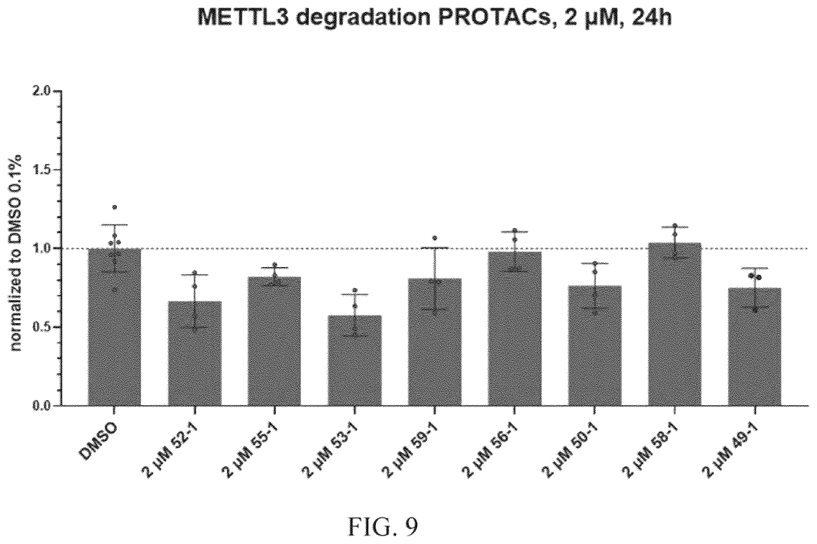

FIG. 9 shows the degradation of METTL3 protein after treatment with 2 μM concentration of various PROTAC molecules for 24h, measured with Western blot.

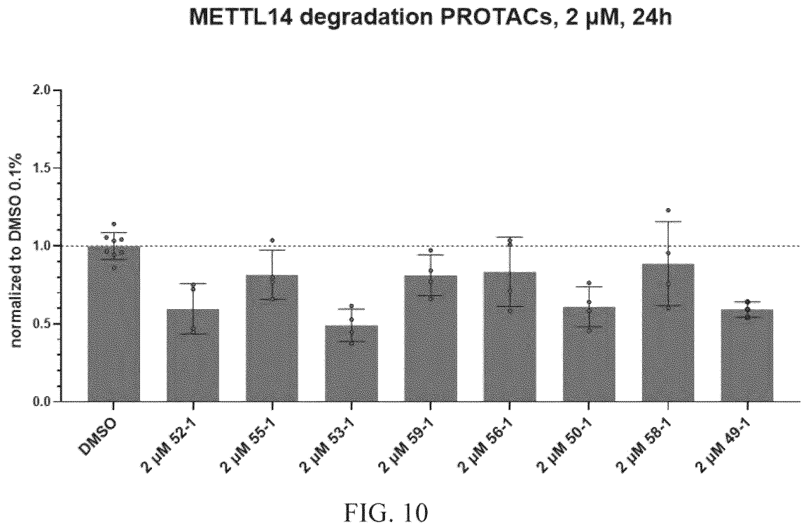

FIG. 10 shows the METTL14 protein after treatment with 2 μM concentration of various PROTAC molecules for 24h, measured with Western blot.

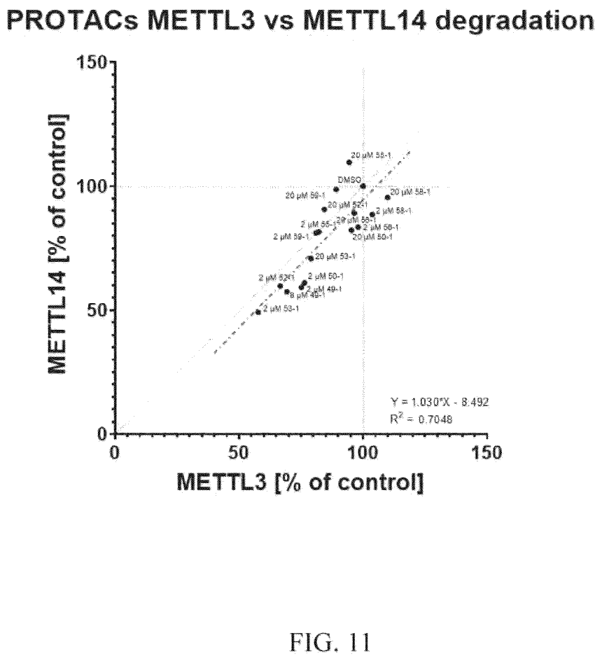

FIG. 11 shows the correlation between METTL3 and METTL14 degradation of various PROTAC molecules measured by Western blot.

1. A compound of the general formula (A), or a pharmaceutically acceptable salt thereof, 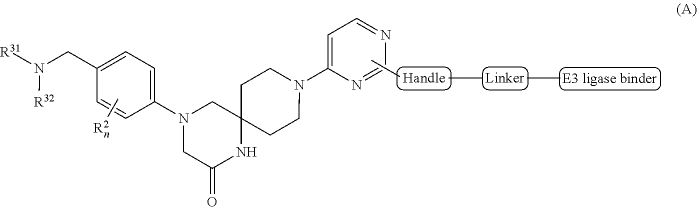
wherein
NR³¹R³² is selected from 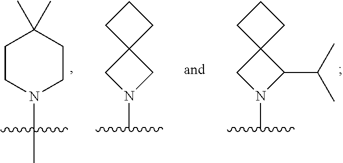
each R² is independently selected from the group comprising F, Cl, CF₃, CHF₂, CH₂F, particularly each R² is F;
n is an integer selected from 0, 1, 2, 3, and 4, particularly n is an integer selected from 0, 1, and 2, more particularly n is 2;
Handle is a connecting moiety comprising or essentially consisting of 3 to 10 atoms of atomic mass ≥12 (C, N, O, S), particularly 4 to 8 atoms of atomic mass ≥12;
Linker is a linker moiety comprising or essentially consisting of 3 to 50 atoms of atomic mass ≥12, particularly 4 to 30 atoms of atomic mass ≥12, more particularly 5 to 20 atoms of atomic mass ≥12;
E3 ligase binder is a moiety specifically binding to an E3 ligase.

2. The compound according to claim 1, or a pharmaceutically acceptable salt thereof, wherein the E3 ligase binder is of the formula (B) 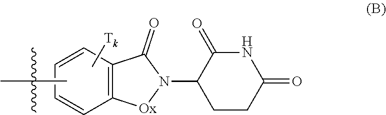
wherein
Ox is CH₂ or C═O;
T is selected from the group comprising F, Cl, particularly T is F;
k is an integer selected from the group comprising 0, 1, 2, particularly the group comprising 0, 1, more particularly k is 0; [symbol]
designates the bond to the Linker.

3. The compound according to claim 2, or a pharmaceutically acceptable salt thereof, wherein k is 0.

4. The compound according to any one of the preceding claims, or a pharmaceutically acceptable salt thereof, wherein the Handle comprises or essentially consists of 1, 2, 3, or 4 chemical moieties selected from the group comprising alkyl, amine, phenyl, and carbonyl.

5. The compound according to any one of the preceding claims, or a pharmaceutically acceptable salt thereof, wherein the Handle is selected from the group comprising the following formulas: 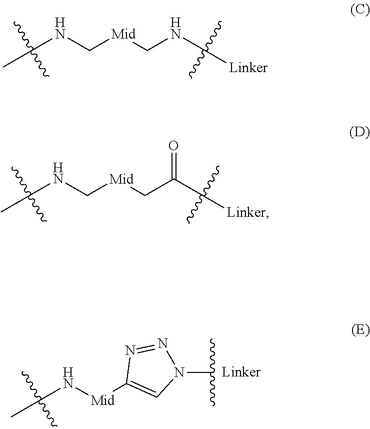
wherein
Mid is selected from the group comprising C₁-C₃ alkyl, and phenyl.

6. The compound according to any one of the preceding claims, or a pharmaceutically acceptable salt thereof, wherein the Handle is selected from the group comprising the following formulas:

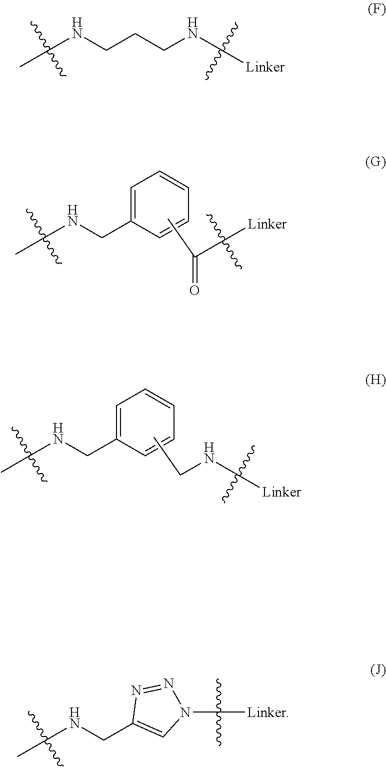

7. The compound according to any one of claims 1-4, or a pharmaceutically acceptable salt thereof, wherein the Handle is Formula (X): 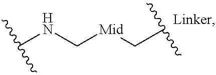
wherein Mid is selected from the group comprising C₁-C₃ alkyl, and phenyl.

8. The compound of claim 7, or a pharmaceutically acceptable salt thereof, wherein the Handle is Formula (Y):

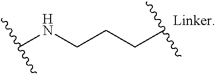 [SMILES: *CCCN*]

9. The compound according to any one of the preceding claims, or a pharmaceutically acceptable salt thereof, wherein the Linker comprises or essentially consists of 1, 2, 3, 4, 5, 6, or 7 chemical moieties independently selected from the group comprising alkyl, aryl, heteroaryl, cycloalkyl, heterocycloalkyl, alkylene, alkylyne, ethylene glycol, carbonyl, ether, ester, amine, amide, sulfonamide, wherein the chemical moieties are each independently unsubstituted or substituted with C₁-C₃ alkyl, halogen, CN, NO₂, hydroxyl, amine, sulfate, phosphate, and/or carboxyl;
particularly wherein the Linker comprises or essentially consists of 1, 2, 3, or 4 chemical moieties selected from the group comprising alkyl, ethylene glycol, carbonyl, piperazine, aryl, amine, triazole.

10. The compound according to any one of the preceding claims, or a pharmaceutically acceptable salt thereof, wherein the Linker is selected from the group comprising the following formulas: 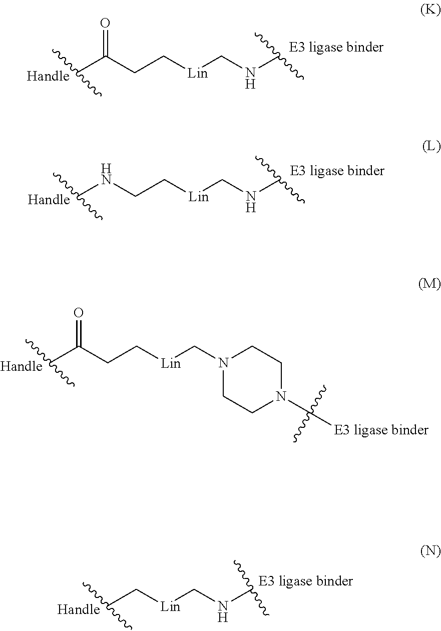
wherein
Lin is selected from the group comprising C₃-C₂₀ alkyl, C₃-C₂₀ alkyl-triazole, oligo(ethylene glycol).

11. The compound according to any one of the preceding claims, or a pharmaceutically acceptable salt thereof, wherein the Linker is selected from the group comprising the following formulas: 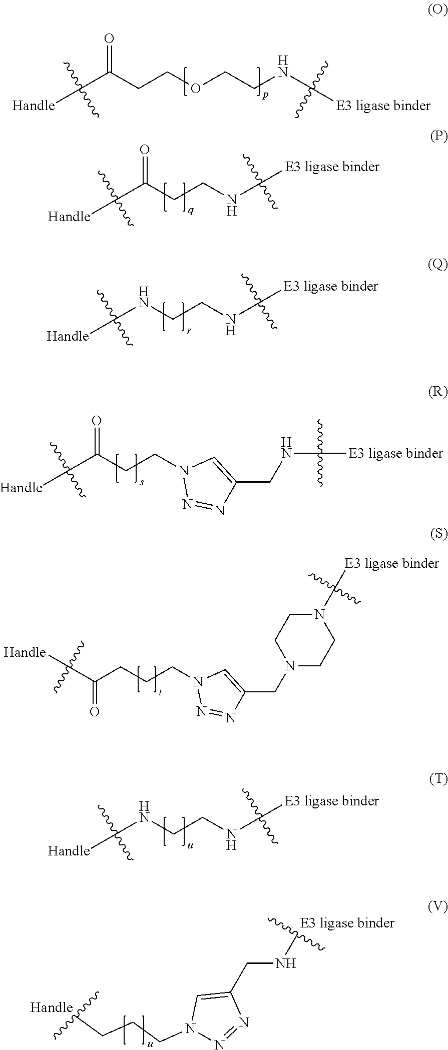
wherein
p is selected from 2, 3, 4, and 5;
q is selected from 7, 8, 9, 10, 11, 12, and 13;
r is selected from 11, 12, 13, 14, 15, 16, and 17;
s is selected from 7, 8, 9, 10, 11, 12, and 13;
t is selected from 3, 4, 5, 6, 7, 8, and 9;
u is selected from 7, 8, 9, 10, 11, 12, 13, 14, 15, 16, 17, 18, 19, or 20.

12. The compound according to claim 11, or a pharmaceutically acceptable salt thereof, wherein
p is selected from 2, 3, 4, and 5;
q is selected from 7, 8, 9, 10, 11, 12, and 13;
r is selected from 11, 12, 13, 14, 15, 16, and 17;
s is selected from 7, 8, 9, 10, 11, 12, and 13;
t is selected from 3, 4, 5, 6, 7, 8, and 9; and
u is selected from 7, 8, 9, 10, 11, 12, and 13.

13. The compound according to any one of claims 1-9, or a pharmaceutically acceptable salt thereof, wherein the Linker comprises Formula (W): 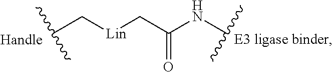
wherein Lin is selected from the group comprising C₃-C₂₀ alkyl, C₃-C₂₀ alkyl-triazole, and oligo(ethylene glycol).

14. The compound according to claim 13, or a pharmaceutically acceptable salt thereof, wherein the Linker comprises Formula (Z):

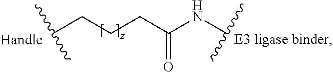

15. wherein z is selected from 4, 5, 6, 7, 8, 9, and 10. The compound according to any one of the preceding claims 1 to 6, or a pharmaceutically acceptable salt thereof, wherein the Linker is a peptide, particularly a peptide consisting of proteinogenic amino acids.

16. The compound according to any one of the preceding claims, or a pharmaceutically acceptable salt thereof, wherein
the E3 ligase binder is of the formula (B) as defined in claim 2; and
the Handle has the definition of claim 6; and
the Linker has the definition of claim 11 or 12.

17. The compound according to any one of the preceding claims, or a pharmaceutically acceptable salt thereof, wherein
the E3 ligase binder is of the formula (B) as defined in claim 2; and
the Handle has the definition of claim 7 or 8; and
the Linker has the definition of claim 11 or 12.

18. The compound according to any one of the preceding claims, or a pharmaceutically acceptable salt thereof, wherein
the E3 ligase binder is of the formula (B) as defined in claim 2; and
the Handle has the definition of claim 5; and
the Linker has the definition of claim 13 or 14.

19. The compound according to any one of the preceding claims, or a pharmaceutically acceptable salt thereof, wherein the compound comprises the following definitions of the Handle, Linker and E3 ligase binder:

| Handle | Linker | E3 ligase binder |
|---|---|---|
| formula (F) | formula (O) | 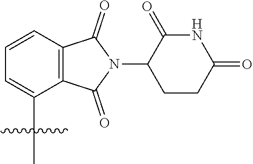 [SMILES: *c1cccc2c1C(=O)N(C1CCC(=O)NC1=O)C2=O] |
| formula (F) | formula (R) | 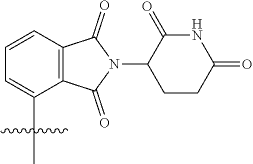 [SMILES: *c1cccc2c1C(=O)N(C1CCC(=O)NC1=O)C2=O] |
| | | 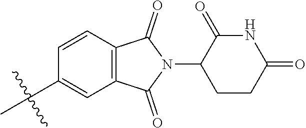 [SMILES: *c1ccc2c(c1)C(=O)N(C1CCC(=O)NC1=O)C2=O] |
| formula (F) | formula (P) | 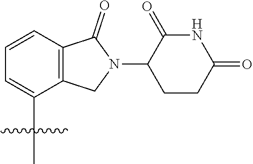 [SMILES: *c1cccc2c1CN(C1CCC(=O)NC1=O)C2=O] |
| | | 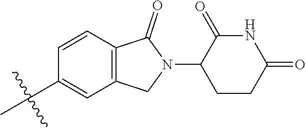 [SMILES: *c1ccc2c(c1)CN(C1CCC(=O)NC1=O)C2=O] |
| formula (F) | formula (S) | 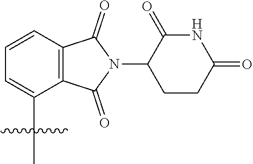 [SMILES: *c1cccc2c1C(=O)N(C1CCC(=O)NC1=O)C2=O] |
| | | 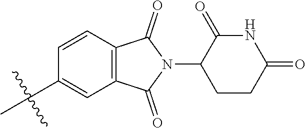 [SMILES: *c1ccc2c(c1)C(=O)N(C1CCC(=O)NC1=O)C2=O] |
| formula (H) | formula (P) | 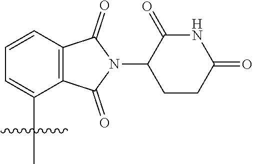 [SMILES: *c1cccc2c1C(=O)N(C1CCC(=O)NC1=O)C2=O] |
| | | 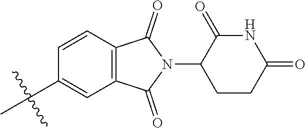 [SMILES: *c1ccc2c(c1)C(=O)N(C1CCC(=O)NC1=O)C2=O] |
| Formula (H) | Formula (R) | 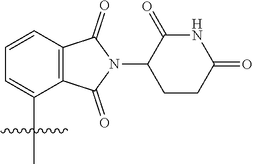 [SMILES: *c1cccc2c1C(=O)N(C1CCC(=O)NC1=O)C2=O] |
| | | 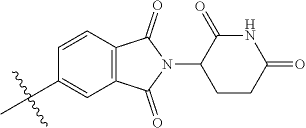 [SMILES: *c1ccc2c(c1)C(=O)N(C1CCC(=O)NC1=O)C2=O] |
| formula (G) | formula (Q) | 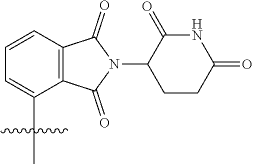 [SMILES: *c1cccc2c1C(=O)N(C1CCC(=O)NC1=O)C2=O] |
| | | 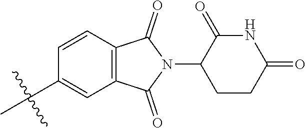 [SMILES: *c1ccc2c(c1)C(=O)N(C1CCC(=O)NC1=O)C2=O] |
| formula (J) | formula (T) | 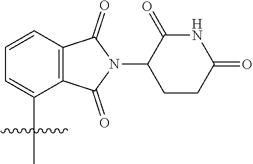 [SMILES: *c1cccc2c1C(=O)N(C1CCC(=O)NC1=O)C2=O] |
| | | 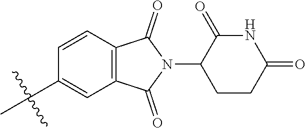 [SMILES: *c1ccc2c(c1)C(=O)N(C1CCC(=O)NC1=O)C2=O] |

20. The compound according to any one of the preceding claims 1 to 19, or a pharmaceutically acceptable salt thereof, wherein NR³¹R³² is

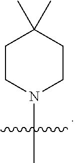 [SMILES: *N1CCC(C)(C)CC1]

21. The compound according to any one of the preceding claims 1 to 19, or a pharmaceutically acceptable salt thereof, wherein NR³¹R³² is

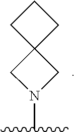 [SMILES: *N1CC2(CCC2)C1]

22. The compound according to any one of the preceding claims 1 to 19, or a pharmaceutically acceptable salt thereof, wherein NR³¹R³² is 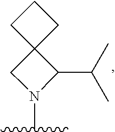 [SMILES: *N1CC2(CCC2)C1C(C)C]
particularly NR³¹R³² is

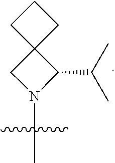 [SMILES: *N1CC2(CCC2)[C@@H]1C(C)C]

23. A compound selected from Tables 2 and 3, or a pharmaceutically acceptable salt thereof.

24. A compound according to any of the preceding claims or a pharmaceutically acceptable salt thereof for use as a medicament.

25. A compound according to any of the preceding claims 1 to 23 or a pharmaceutically acceptable salt thereof for use in treatment of cancer.

26. The compound for use according to claim 25, or a pharmaceutically acceptable salt thereof, wherein said cancer is selected from the group comprising renal cancer, breast cancer, acute myeloid leukemia, hepatocellular carcinoma, and lung adenocarcinoma.
